(19) United States
(12) Patent Application Publication
(10) Pub. No.: US 2025/0313574 A1
(43) Pub. Date: Oct. 9, 2025

(54) SIALIC ACID DERIVATIVES AND METHODS OF USING SAME

(71) Applicant: EISAI R&D MANAGEMENT CO., LTD., Tokyo (JP)

(72) Inventors: Lynn D. HAWKINS, Cambridge, MA (US); Branko MITASEV, Cambridge, MA (US); Charles CHASE, Cambridge, MA (US); Jaemoon LEE, Cambridge, MA (US); Jung Hwa LEE, Cambridge, MA (US); Danyang LI, Cambridge, MA (US); Matthew SCHNADERBECK, Cambridge, MA (US); Mingde David SHAN, Cambridge, MA (US); Robert Tzu Hsiang YU, Cambridge, MA (US); Wanjun ZHENG, Cambridge, MA (US); Xiaojie ZHU, Cambridge, MA (US); Junko ARAI, Cambridge, MA (US); Francis G. FANG, Cambridge, MA (US); John WANG, Cambridge, MA (US)

(21) Appl. No.: 18/873,059

(22) PCT Filed: Jun. 9, 2023
(86) PCT No.: PCT/US2023/024929

§ 371 Date: Dec. 9, 2024

Related U.S. Application Data
(60) Provisional application No. 63/366,211, filed on Jun. 10, 2022.

(51) Int. Cl. C07D 498/10; A61K 31/416; A61K 31/4439; A61K 31/499; A61K 31/5386; C07D 401/12; C07D 405/12; C07D 405/14; C07D 407/12; C07D 413/10; C07D 417/12; C07D 471/10; C07D 487/10

(52) U.S. Cl. CPC C07D 498/10; A61K 31/416; A61K 31/4439; A61K 31/499; A61K 31/5386; C07D 401/12; C07D 405/12; C07D 405/14; C07D 407/12; C07D 413/10; C07D 417/12; C07D 471/10; C07D 487/10

(57) ABSTRACT

Compound of Formula (I)-(V), compositions comprising at least one compound chosen from compounds of Formula (I)-(V), and methods of using the same, including in treatment of Alzheimer's disease.

Alzheimer's disease (AD) is a complicated neurodegenerative disease with progressive cognitive impairment common in elderly people. In 2006, the prevalence of AD was 26.6 million around the world, and the number of AD will quadruple by 2050. (Ziegler-Graham K., et al. “Forecasting the global burden of Alzheimer's disease,” Alzheimers Dement. 2007; 3:186-91.). AD consists of early-onset AD (EOAD) and late-onset AD (LOAD). LOAD, which accounts for the majority of AD, is the result of interaction between environmental and genetic factors (Lu Zy et al., “Spreading of Pathology in Alzheimer's Disease,” Neurotox Res 2017; 32:707-22.). Genetic factors play an important role in it, and the heritability is estimated to be up to 80% (Gatz M. et al., “Role of genes and environments for explaining Alzheimer disease,” Arch Gen Psychiatry 2006; 63:168-74; Palotas A, et al. “Candidate susceptibility genes in Alzheimer's disease are at high risk for being forgotten—they don't give peace of mind,” Curr Drug Metab 2006; 7:273-93; Antoniades D. et al., “The role of reelin gene polymorphisms in the pathogenesis of Alzheimer's disease in a Greek population,” J Biol Regul Homeost Agents 2011; 25:351-8; Wang L. Z. et al., “Association between late-onset Alzheimer's disease and microsatellite polymorphisms in intron II of the human toll-like receptor 2 gene,” Neurosci Lett 2011; 489:164-7.)

Up to now, the apolipoprotein E (APOE) gene is the only gene recognized to increase the risk of LOAD, but that gene only accounts for 27.3% about the risk of AD onset (Hollingworth P. et al. “Common variants in ABCA7, MS4A6A/MS4A4E, EPHA1, CD33 and CD2AP are associated with Alzheimer's disease,” Nat Genet 2011; 43:429-35; Jayadev S. et al., “Alzheimer's disease phenotypes and genotypes associated with mutations in presenilin 2,” Brain 2010; 133:1143-54; Alzheimer's Association “2015 Alzheimer's disease facts and figures.” Alzheimers Dement 2015; 11:332-84; Liu Y. et al., “Multiple Effect of APOE Genotype on Clinical and Neuroimaging Biomarkers Across Alzheimer's Disease Spectrum,” Mol Neurobiol 2016; 53:4539-47). Therefore, further efforts are needed to look for risk genes other than APOE.

Preclinical experimental evidence reported by two research groups supports the potential role of the sialic acid-binding site of CD33 and its relationship to microglial cell activation and AD. The use of a CD33 ligand bound to microparticles was found to increase the uptake of amyloid-β (Aβ), into microglial cells (Miles L. A. et al., “Small Molecule Binding to Alzheimer Risk Factor CD33 Promotes Aβ Phagocytosis,” iScience 2019; 19:110-118.) Confirmation of this result was reported using a CD33 ligand bound to a lipid followed by incorporation into a liposome (Bhattacherjee A. et al., “Increasing phagocytosis of microglia through targeting CD33 with liposomes displaying glycan ligands,” J Controlled Release 2021; 338:680-693.) These results provide good evidence that a ligand, which strongly binds to the sialic acid-binding site on CD33 is a promising therapy that would promote clearance of the Aβ, and thus may have an impact on AD.

Provided herein are sialic acid derivatives which may be useful for treatment and/or prevention of Alzheimer's disease.

One aspect of the disclosure provides compounds of Formula (I), (Ia), (Ib), (Ic), (Id), (II), (IIa), (III), (IV), and (V), tautomers thereof, deuterated derivatives, and pharmaceutically acceptable salts of any of the foregoing, which may be useful in the treatment of AD. For example, a compound can be chosen from compounds of Formula (I):

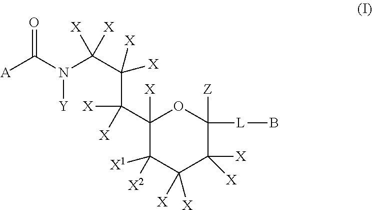

a tautomer thereof, a deuterated derivative of a compound of Formula (I), a deuterated derivative of a tautomer of a compound of Formula (I), or a pharmaceutically acceptable salt of any of the foregoing, wherein:
A is chosen from alkenyl groups, cycloalkyl groups, heterocyclic groups, aryl groups, and heteroaryl groups.
B is chosen from hydrogen,

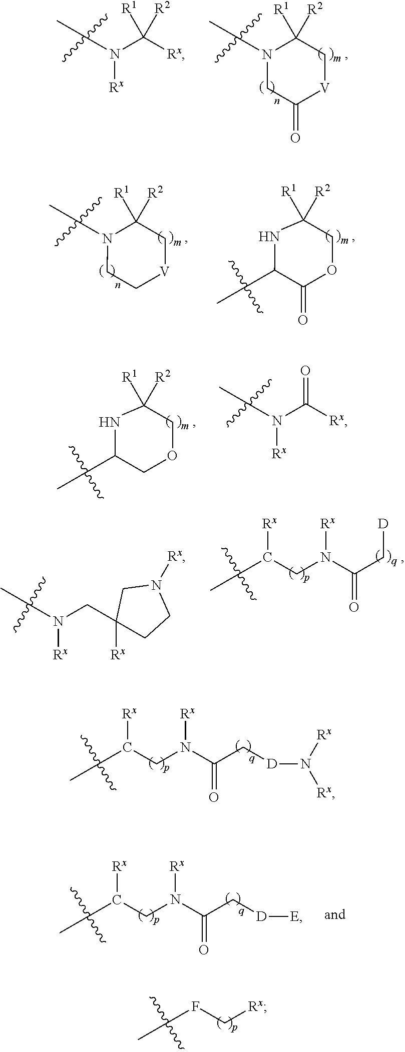

wherein
   V is chosen from O, CH₂ and NR′; wherein R′ is chosen from hydrogen, hydroxy groups, linear alkyl groups, branched alkyl groups, and cyclic alkyl groups;
   R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
   each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
L is chosen from C₁₋₁₀ linear alkylene groups, C₁₋₁₀ branched alkylene groups, and C₁₋₁₀ cyclic alkylene groups, —C(O)—C₁₋₁₀ linear alkylene groups, C₁₋₁₀ branched alkylene groups, and C₁₋₁₀ cyclic alkylene groups, C₁₋₁₀ linear alkylene-C(O)— groups, C₁₋₁₀ branched alkylene-C(O)— groups, and C₁₋₁₀ cyclic alkylene-C(O)— groups, C₁₋₁₀ linear alkenylene groups, C₁₋₁₀ branched alkenylene groups, and C₁₋₁₀ cyclic alkenylene groups,

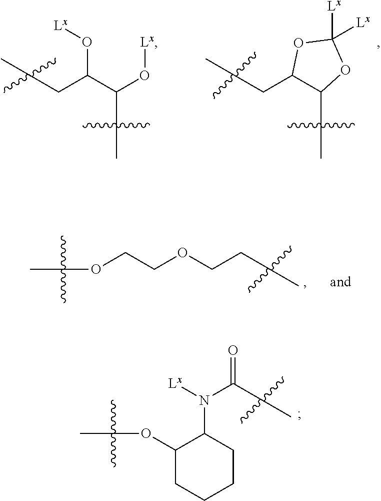

wherein each L^{x} is independently chosen from hydrogen, linear alkyl groups, branched alkyl groups, and cyclic alkyl groups;
each X is independently chosen from hydrogen, hydroxy groups, amino groups, linear alkyl groups, branched alkyl groups, and cyclic alkyl groups;
X¹ and X² are each independently chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
Y is chosen from hydrogen, linear alkyl groups, branched alkyl groups, and cyclic alkyl groups;
Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC₃-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

In one aspect of the disclosure, the compounds of Formula IIa can be chosen from:

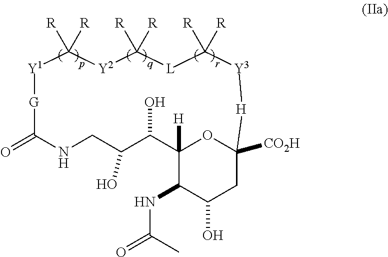

a tautomer thereof, a deuterated derivative of a compound of Formula (IIa), a deuterated derivative of a tautomer of a compound of Formula (IIa), or a pharmaceutically acceptable salt of any of the foregoing, wherein:
G is chosen from cycloalkyl groups, heterocyclic groups, aryl groups, and heteroaryl groups;
Y¹ is absent or —O—;
Y² is absent or chosen from —O—, —NHC(O)—, and aryl groups;
Y³ is absent or chosen from —O—, and aryl groups;
H is chosen from C₁₋₁₀ linear alkylene groups, C₃₋₁₀ branched alkylene groups, C₃₋₁₀ cyclic alkylene groups, —C(O)—C₁₋₁₀ linear alkylene groups, —C(O)—C₃₋₁₀ branched alkylene groups, —C(O)—C₃₋₁₀ cyclic alkylene groups, C₁₋₁₀ linear alkylene-C(O)— groups, C₃₋₁₀ branched alkylene-C(O)— groups, C₃₋₁₀ cyclic alkylene-C(O)— groups, C₁₋₁₀ linear alkenylene groups, C₃₋₁₀ branched alkenylene groups, and C₃₋₁₀ cyclic alkenylene groups;
p, q, and r are independently chosen from 0, 1, 2, 3, 4, 5, and 6;
each R is independently chosen from hydrogen, hydroxy groups, amino groups, linear, branched, and cyclic alkyl groups, linear, branched, and cyclic alkoxy groups;
L absent or is chosen from:

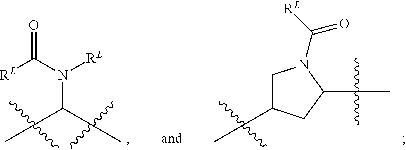

wherein R^{L} is chosen hydrogen, linear alkyl groups, branched alkyl groups, cyclic alkyl groups, heterocyclic groups, aryl groups, and heteroaryl groups;
wherein the linear alkyl groups, branched alkyl groups, and cyclic alkyl groups, linear alkenyl groups, branched alkenyl groups, and cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

In one aspect of the disclosure, the compounds of Formulas (I), (Ia), (Ib), (Ic), (Id), (II) are further derivatized to yield compounds of Formulas (III), (IV), (V), a tautomer thereof, a deuterated derivative of the compound or the tautomer, or a pharmaceutically acceptable salt of the foregoing. For example, a compound of Formula (III), (IV), or (V) is chosen from:

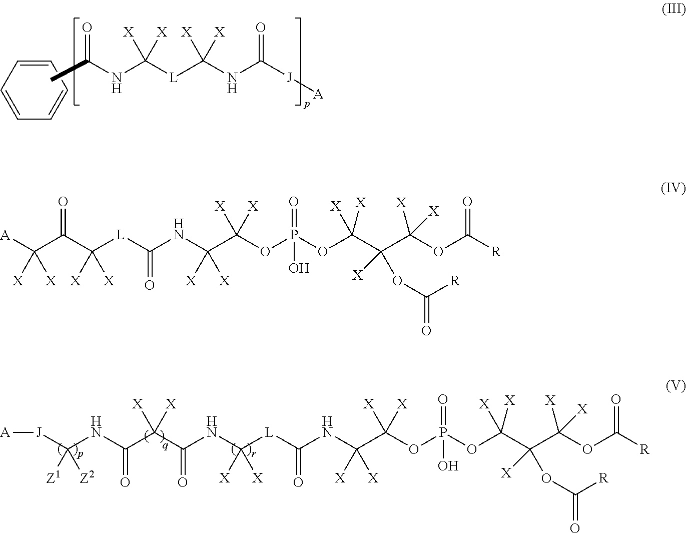

a tautomer thereof, a deuterated derivative of a compound of Formula (III), (IV), or (V), a deuterated derivative of a tautomer of a compound of Formula (III), (IV), or (V), or a pharmaceutically acceptable salt of any of the foregoing, wherein:
A is a compound of Formula (I), (Ia), (Ib), (Ic), (Id), or (II);
J is chosen from cycloalkyl groups, heterocyclic groups, aryl groups, and heteroaryl groups;
Z¹, Z², and each X are each independently chosen from hydrogen, hydroxy groups, amino groups, linear alkyl groups, branched alkyl groups, and cyclic alkyl groups;
L is

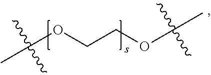

wherein s is 1-50;
p, q, and r are independently chosen from 1, 2, 3, 4, 5, and 6.

In some embodiments, the disclosure provides pharmaceutical compositions comprising at least one compound of Formula (I), (Ia), (Ib), (Ic), (Id), (II), (III), (IV), (V), a tautomer thereof, a deuterated derivative of the compound or the tautomer, or a pharmaceutically acceptable salt of the foregoing. These compositions may further include at least one additional active pharmaceutical ingredient and/or at least one carrier.

Another aspect of the disclosure provides methods of treating AD comprising administering to a subject in need thereof, at least compound of Formula (I), (Ia), (Ib), (Ic), (Id), (II), (III), (IV), (V), a tautomer thereof, a deuterated derivative of the compound or the tautomer, or a pharmaceutically acceptable salt of the foregoing or a pharmaceutical composition comprising the at least compound.

In some embodiments, the methods of treatment include administration of at least one additional active agent to the subject in need thereof, either in the same pharmaceutical composition as the at least compound of Formula (I), (Ia), (Ib), (Ic), (Id), (II), (III), (IV), (V), a tautomer thereof, a deuterated derivative of the compound or the tautomer, or a pharmaceutically acceptable salt of the foregoing, or as separate compositions.

### BRIEF DESCRIPTION OF THE DRAWINGS

FIG. 1 depicts thermograms (A) and derivative curves (B) of His-CD33 (gray) and His-CD33 with A-001 (blue).

FIG. 2 depicts the phagocytosis of lipid formulations at 100 μM in macrophages from hCD33 mice at Day 1. Each formulation contains 0.1% 0.1% AF647PEG-DSPE.

FIG. 3 depicts the phagocytosis of lipid formulations at 100 μM in macrophages from hCD33 mice at Day 2. Each formulation contains 0.1% 0.1% AF647PEG-DSPE.

FIG. 4 depicts Figure phagocytosis of lipid formulations at 10 μM in macrophages from hCD33 mice at Day 2.

### DEFINITIONS

The following are definitions of terms used in the present application.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B,” when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When a number is recited, either alone or as part of a numerical range, it should be understood that the numerical value can vary above and below the stated value by a variance of 10% of the stated value.

As used herein, “optionally substituted” is interchangeable with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms.

For example, compounds having the present structures except for the replacement of hydrogen by deuterium or tritium, or the replacement of a carbon by a 13C or 14C are within the scope of this disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “2H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at least 6333.3 (95% deuterium incorporation, at least 6466.7 (97% deuterium incorporation, or at least 6600 (99% deuterium incorporation).

The term “isotopic enrichment factor” as used herein means the ratio between the isotopic abundance and the natural abundance of a specified isotope.

The term “alkyl” or “aliphatic” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic that has a single point of attachment to the rest of the molecule. Unless otherwise specified, alkyl groups contain 1 to 20 alkyl carbon atoms. In some embodiments, alkyl groups contain 1 to 10 aliphatic carbon atoms. In some embodiments, alkyl groups contain 1 to 8 aliphatic carbon atoms. In some embodiments, alkyl groups contain 1 to 6 alkyl carbon atoms, and in some embodiments, alkyl groups contain 1 to 4 alkyl carbon atoms, and in yet other embodiments alkyl groups contain 1 to 3 alkyl carbon atoms. Nonlimiting examples of alkyl groups include, but are not limited to, linear or branched, and substituted or unsubstituted alkyl. Suitable cycloaliphatic groups include cycloalkyl, bicyclic cycloalkyl (e.g., decalin), bridged bicycloalkyl such as norbornyl or [2.2.2]bicyclo-octyl, or bridged tricyclic such as adamantyl. In some embodiments, alkyl groups are substituted. In some embodiments, alkyl groups are unsubstituted.

In some embodiments, alkyl groups are straight-chain. In some embodiments, alkyl groups are branched.

The terms “cycloalkyl,” “carbocycle,” “cycloaliphatic,” or “cyclic alkyl” refer to a spirocyclic or monocyclic C3-8 hydrocarbon or a spirocyclic, bicyclic, bridged bicyclic, tricyclic, or bridged tricyclic C8-14 hydrocarbon that is completely saturated or that contains one or more units of unsaturation, but which is not aromatic, wherein any individual ring in said bicyclic ring system has 3 to 7 members. In some embodiments, cyclogroups are substituted. In some embodiments, cyclogroups are unsubstituted.

The term “heteroalkyl,” or “heteroaliphatic” as used herein, means aliphatic groups wherein one or two carbon atoms are independently replaced by one or more of oxygen, sulfur, nitrogen, phosphorus, or silicon. Heteroaliphatic groups may be substituted or unsubstituted, branched or unbranched, cyclic or acyclic, and include “heterocycle”, “heterocyclyl”, “heterocycloaliphatic”, or “heterocyclic” groups.

The term “alkenyl” as used herein, means a straight-chain (i.e., unbranched), branched, substituted or unsubstituted hydrocarbon chain that contains one or more units of saturation, or a monocyclic hydrocarbon or bicyclic hydrocarbon that contains one or more units of unsaturation, but which is not aromatic (referred to herein as, “cyclic alkenyl”). In some embodiments, alkenyl groups are substituted. In some embodiments, alkenyl groups are unsubstituted. In some embodiments, alkenyl groups are straight-chain. In some embodiments, alkenyl groups are branched.

The term “heterocycle”, “heterocyclyl”, “heterocycloaliphatic”, or “heterocyclic” as used herein means non-aromatic, monocyclic, bicyclic, or tricyclic ring systems in which one or more ring members is an independently chosen heteroatom. In some embodiments, the “heterocycle”, “heterocyclyl”, “heterocycloaliphatic”, or “heterocyclic” group has 3 to 14 ring members in which one or more ring members is a heteroatom independently chosen from oxygen, sulfur, nitrogen, and phosphorus. In some embodiments, each ring in a bicyclic or tricyclic ring system contains 3 to 7 ring members. In some embodiments the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

The term “unsaturated”, as used herein, means that a moiety has one or more units or degrees of unsaturation. Unsaturation is the state in which not all of the available valance bonds in a compound are satisfied by substituents and thus the compound contains double or triple bonds.

The term “alkoxy”, or “thioalkyl”, as used herein, refers to an alkyl group, as previously defined, wherein one carbon of the alkyl group is replaced by an oxygen (“alkoxy”) or sulfur (“thioalkyl”) atom, respectively, provided that the oxygen and sulfur atoms are linked between two carbon atoms. A “cyclic alkoxy” refers to a monocyclic, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl. In some embodiments, “alkoxy” and/or “thioalkyl” groups are substituted. In some embodiments, “alkoxy” and/or “thioalkyl” groups are unsubstituted.

The terms “haloalkyl” and “haloalkoxy,” as used herein, means a linear or branched alkyl or alkoxy, as the case may be, which is substituted with one or more halogen atoms. Non-limiting examples of haloalkyl groups include CHF₂, CH₂F, CF₃, CF₂, and perhaloalkyls, such as CF₂CF₃. Non-limiting examples of haloalkoxy groups include —OCHF₂, —OCH₂F, —OCF₃, and —OCF₂—.

The term “halogen” includes F, Cl, Br, and I, i.e., fluoro, chloro, bromo, and iodo, respectively.

The term “aminoalkyl” means an alkyl group which is substituted with or contains an amino group.

As used herein, an “amino” refers to a group which is a primary, secondary, or tertiary amine.

As used herein, a “carbonyl” group refers to C═O.

As used herein, a “cyano” or “nitrile” group refer to C≡N.

As used herein, a “hydroxy” or “hydroxyl” group refers to OH.

As used herein, a “thiol” group refers to SH.

As used herein, “tert” and “t-” each refer to tertiary.

As used herein, “aromatic groups” or “aromatic rings” refer to chemical groups that contain conjugated, planar ring systems with delocalized pi electron orbitals comprised of [4n+2]p orbital electrons, wherein n is an integer ranging from 0 to 6. Nonlimiting examples of aromatic groups include aryl and heteroaryl groups.

The term “aryl” used alone or as part of a larger moiety as in “arylalkyl”, “arylalkoxy”, or “aryloxyalkyl”, refers to monocyclic, bicyclic, and tricyclic ring systems having a total of five to fourteen ring members, wherein at least one ring in the system is aromatic and wherein each ring in a bicyclic or tricyclic ring system contains 3 to 7 ring members. The term “aryl” also refers to heteroaryl ring systems as defined herein below. Nonlimiting examples of aryl groups include phenyl rings. In some embodiments, aryl groups are substituted. In some embodiments, aryl groups are unsubstituted.

The term “heteroaryl”, used alone or as part of a larger moiety as in “heteroarylalkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. In some embodiments, heteroaryl groups are substituted. In some embodiments, heteroaryl groups have one or more heteroatoms chosen from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted.

Non-limiting examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999) and 4th Edition (John Wiley & Sons, New Jersey, 2014).

Non-limiting examples of suitable solvents that may be used in this disclosure include, but are not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-MeTHF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge et al., J. Pharmaceutical Sciences, 1977, 66, 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, $-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The terms “patient” and “subject” are used interchangeably and refer to an animal including a human.

The terms “effective dose” and “effective amount” are used interchangeably herein and refer to that amount of compound that produces the desired effect for which it is administered (e.g., improvement in symptoms of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to the following: complete or partial remission, lower risk of kidney failure (e.g. ESRD), and disease-related complications (e.g. edema, susceptibility to infections, or thrombo-embolic events). Improvements in or lessening the severity of any of these symptoms can be readily assessed according to methods and techniques known in the art or subsequently developed.

The terms “about” and “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

### Non-Limiting Embodiments of the Disclosure

Embodiment 1. A compound of Formula (I)

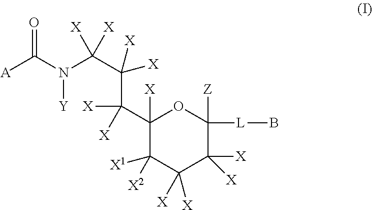

a tautomer thereof, a deuterated derivative of a compound of Formula (I), a deuterated derivative of a tautomer of a compound of Formula (I), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen,

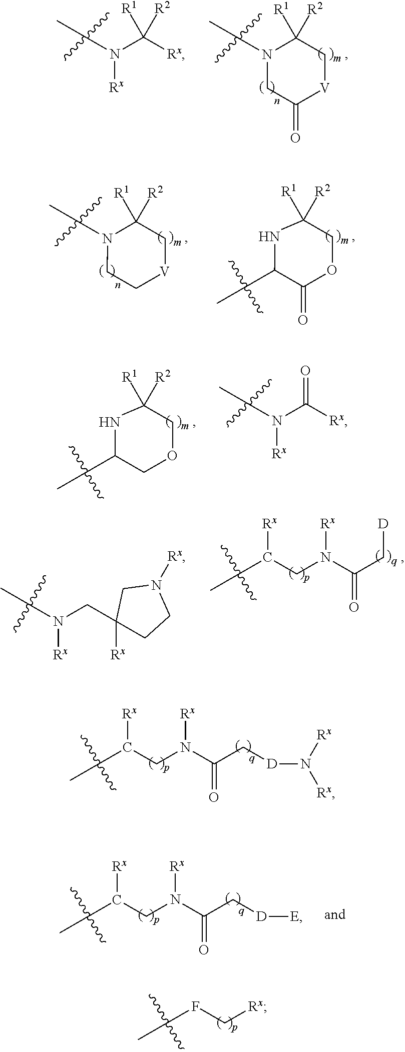

wherein
   V is chosen from O, CH₂ and NR′; wherein R′ is chosen from hydrogen, hydroxy groups, linear alkyl groups, branched alkyl groups, and cyclic alkyl groups;
   R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
   each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear, branched, cyclic alkoxy groups, cycloalkyl groups, heterocycloalkyl groups, aryl groups, and heteroaryl groups;
(iii) L is chosen from C₁₋₁₀ linear alkylene groups, C₁₋₁₀ branched alkylene groups, C₁₋₁₀ cyclic alkylene groups, —C(O)—C₁₋₁₀ linear alkylene groups, C₁₋₁₀ branched alkylene groups, C₁₋₁₀ cyclic alkylene groups, C₁₋₁₀ linear alkylene-C(O)— groups, C₁₋₁₀ branched alkylene-C(O)-groups, C₁₋₁₀ cyclic alkylene-C(O)— groups, C₁₋₁₀ linear alkenylene groups, C₁₋₁₀ branched alkenylene groups, and C₁₋₁₀ cyclic alkenylene groups,

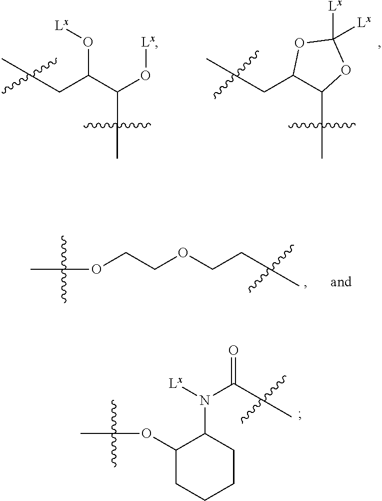

wherein each L^{x} is independently chosen from hydrogen, linear alkyl groups, branched alkyl groups, and cyclic alkyl groups;
(iv) each X is independently chosen from hydrogen, hydroxy groups, amino groups, linear alkyl groups, branched alkyl groups, and cyclic alkyl groups;
(v) X¹ and X² are each independently chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(vi) Y is chosen from hydrogen, linear alkyl groups, branched alkyl groups, and cyclic alkyl groups;
(vii) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein
   R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC₃-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 2. A compound of Formula (Ia):

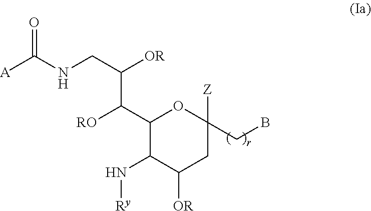

a tautomer thereof, a deuterated derivative of a compound of Formula (Ia), a deuterated derivative of a tautomer of a compound of Formula (Ia), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen,

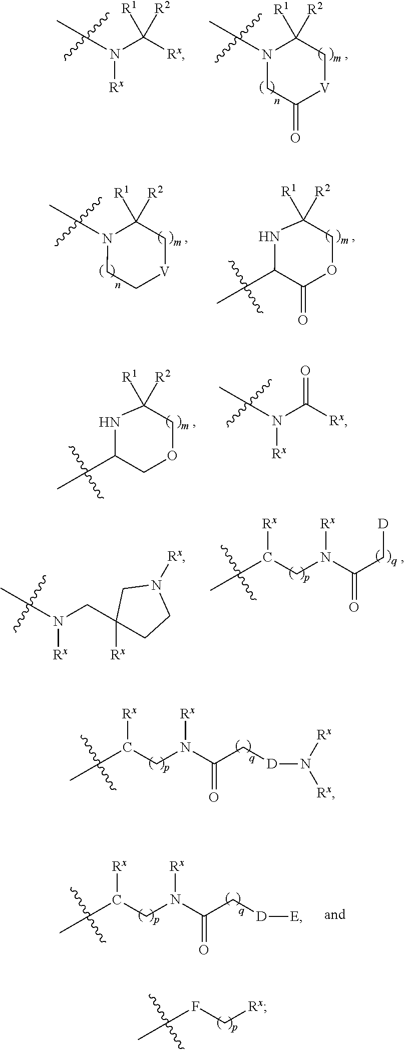

wherein
   V is chosen from O, and NR;
   R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
   each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear, branched, and cyclic alkyl groups;
(iv) R^{y} chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 3. A compound of Formula (Ib):

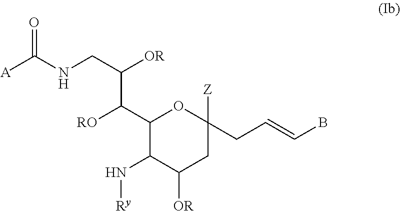

a tautomer thereof, a deuterated derivative of a compound of Formula (Ib), a deuterated derivative of a tautomer of a compound of Formula (Ib), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen,

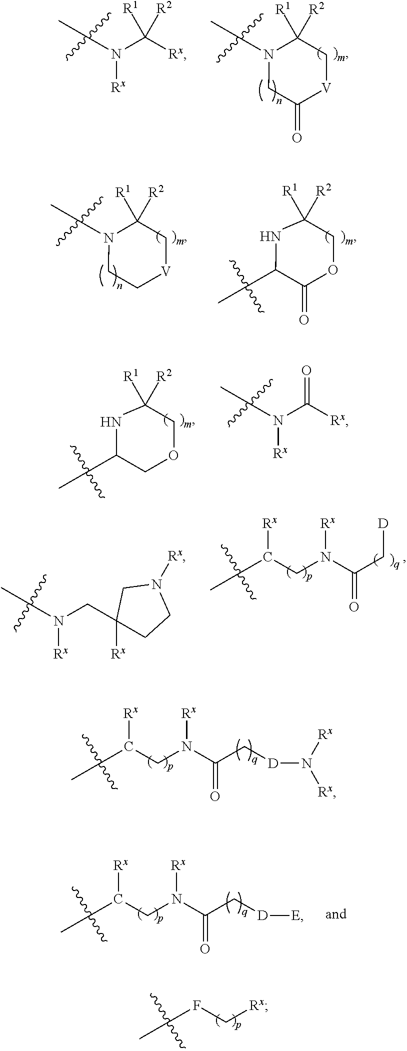

wherein
   V is chosen from O, and NR;
   R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
   each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
   m, n, p, and q are independently chosen from 0, 1, 2, 3, and 4;
   C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear, branched, and cyclic alkyl groups;
(iv) R^{y} chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 4. A compound of Formula (Ic):

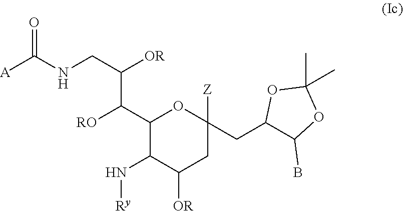

a tautomer thereof, a deuterated derivative of a compound of Formula (Ic), a deuterated derivative of a tautomer of a compound of Formula (Ic), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen,

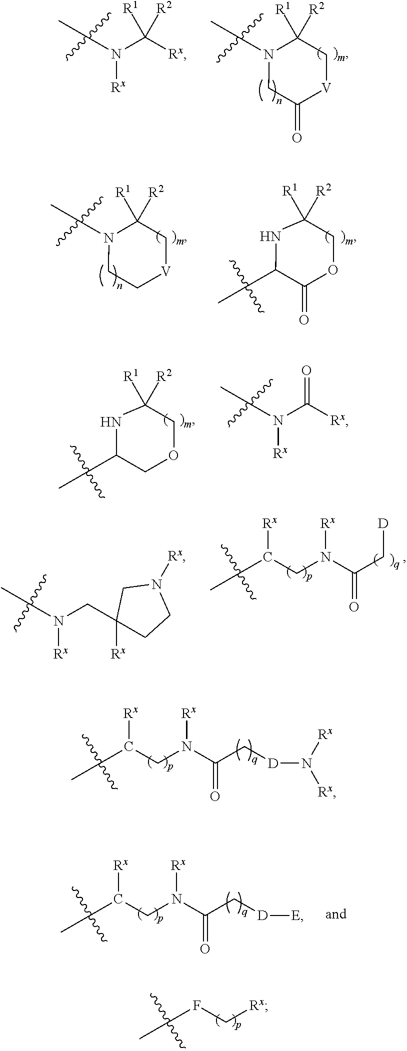

wherein
   V is chosen from O, and NR;
   R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
   each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
   m, n, p, and q are independently chosen from 0, 1, 2, 3, and 4;
   C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear, branched, and cyclic alkyl groups;
(iv) R^{y} chosen from hydrogen, hydroxy groups, amino groups, linear alkyl groups, branched alkyl groups, and cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O— heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 5. A compound of Formula (Id):

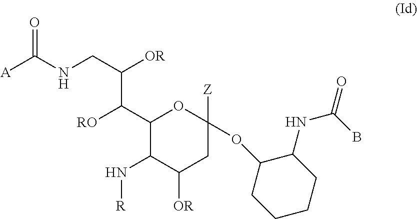

a tautomer thereof, a deuterated derivative of a compound of Formula (Id), a deuterated derivative of a tautomer of a compound of Formula (Id), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen,

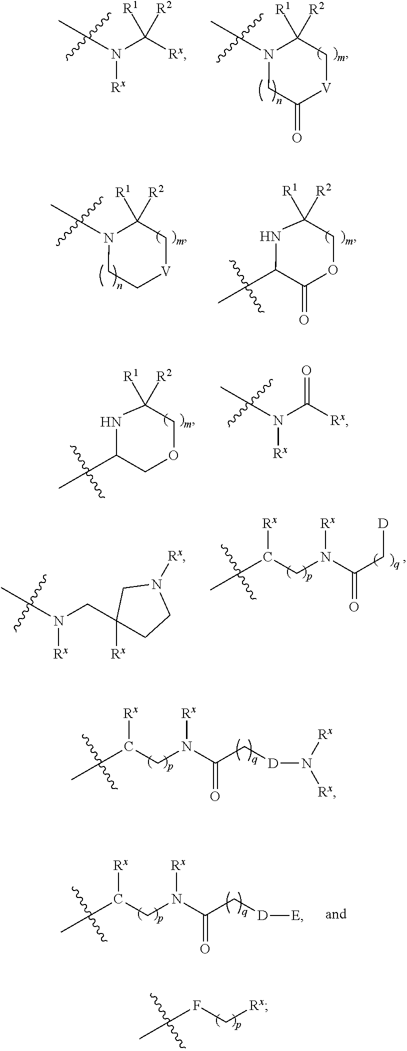

wherein
   V is chosen from O, and NR;
   R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
   each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
   m, n, p, and q are independently chosen from 0, 1, 2, 3, and 4;
   C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear alkyl groups, branched alkyl groups, and cyclic alkyl groups;
(iv) R^{y} chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 6. The compound of any of the preceding claims, wherein A is an aryl group.

Embodiment 7. The compound of claim 6, wherein A is

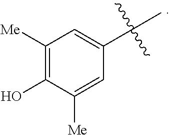 [SMILES: *c1cc(C)c(O)c(C)c1]

Embodiment 8. The compound of any one of claims 1-5, wherein A is an heteroaryl group.

Embodiment 9. The compound of any one of claims 1-5, wherein A is an alkenyl group.

Embodiment 10. The compound of any one of claims 1-5, wherein A is an alkenyl group.

Embodiment 11. The compound of any of the preceding claims, wherein B is

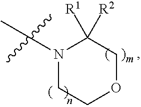

R¹ and R² are each independently chosen from hydrogen, hydroxy groups, amino groups, linear alkyl groups, branched alkyl groups, cyclic alkyl groups or together form a cycloalkyl group or a heterocyclic group; wherein the cycloalkyl group or a heterocyclic group is optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 12. The compound of claim 11, wherein B is

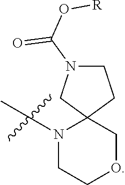

Embodiment 13. The compound of claim 12, wherein R is chosen from linear alkyl groups, branched alkyl groups, and cyclic alkyl groups.

Embodiment 14. The compound of claim 13, wherein R is t-butyl group.

Embodiment 15. The compound of claim 14, wherein B is

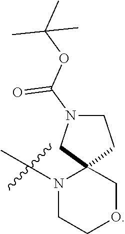

Embodiment 16. The compound of claim 14, wherein B is

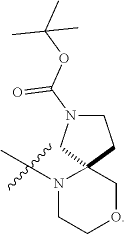

Embodiment 17. The compound of claim 11, wherein B is

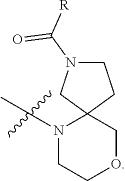

Embodiment 18. The compound of claim 15, wherein R is chosen from linear alkyl groups, branched alkyl groups, and cyclic alkyl groups.

Embodiment 19. The compound of claim 15, wherein R is chosen from aryl groups and heteroaryl groups.

Embodiment 20. The compound of claim 11, wherein B is

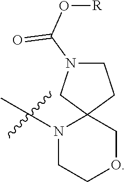

Embodiment 21. The compound of claim 15, wherein R is chosen from linear alkyl groups, branched alkyl groups, and cyclic alkyl groups.

Embodiment 22. The compound of claim 15, wherein R is chosen from aryl groups and heteroaryl groups.

Embodiment 23. The compound of any of claims 1-10, wherein B is

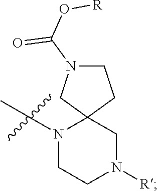

R is chosen from linear alkyl groups, branched alkyl groups, cyclic alkyl groups, aryl groups, and heteroaryl groups; and R′ is chosen from linear alkyl groups, branched alkyl groups, cyclic alkyl groups, —C(O)—C₁-C₆ linear alkyl groups, —C(O)—C₃-C₆ branched alkyl groups, and —C(O)—C₃-C₆cyclic alkyl groups.

Embodiment 24. The compound of claim 24, wherein B chosen from

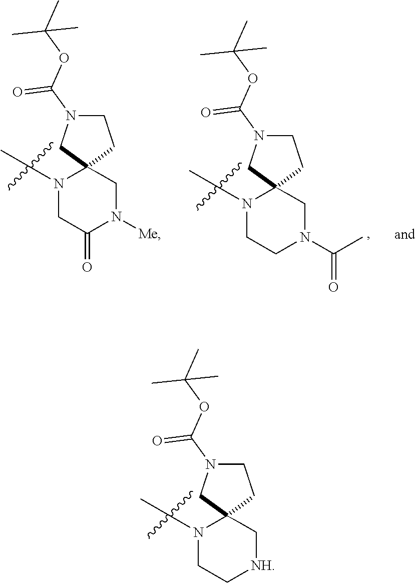

Embodiment 25. The compound of any of claims 1-10, wherein B is

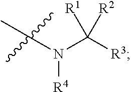

R¹ and R² are each independently chosen from hydrogen, hydroxy groups, linear alkyl groups, branched alkyl groups, and cyclic alkyl groups, or R¹ and R² together form a cycloalkyl group or a heterocyclic group;
R³ and R⁴ are each independently chosen from hydrogen, hydroxy groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups.

Embodiment 26. The compound of claim 25, wherein B is chosen from

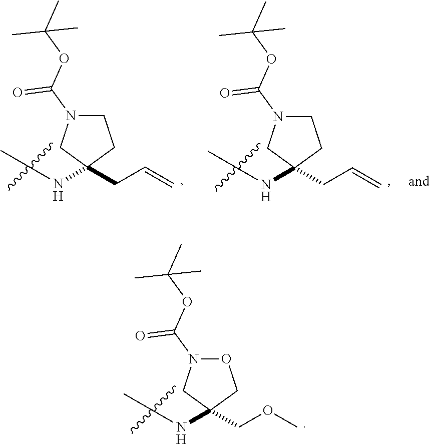

Embodiment 27. The compound of any of claims 1-10, wherein B is

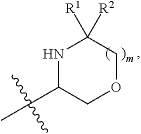

wherein m is 0 or 1; R¹ and R² are each independently chosen from hydrogen, hydroxy groups, linear alkyl groups, branched alkyl groups, cyclic alkyl groups. or R¹ and R² together form a cycloalkyl group or a heterocyclic group.

Embodiment 28. The compound of claim 27, wherein B is chosen from

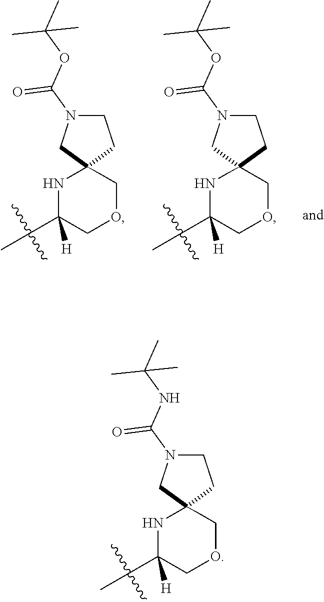

Embodiment 29. The compound of any of claims 1-10, wherein B is

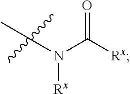

wherein each R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups.

Embodiment 30. The compound of any of claims 1-10, wherein B is

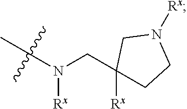

each R^{x} is independently chosen from hydrogen, hydroxy groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups.

Embodiment 31. The compound of any of claims 1-10, wherein B is

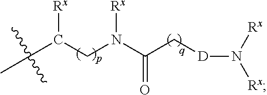

each R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups; p and q are independently chosen from 0, 1, 2, 3, and 4; C and D are independently chosen from linear alkoxy groups, branched alkoxy groups, cyclic alkoxy groups, cycloalkyl groups, heterocycloalkyl groups, aryl groups, and heteroaryl groups.

Embodiment 32. The compound of claim 29, wherein B is

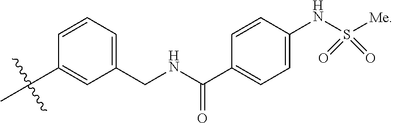 [SMILES: *c1cccc(CNC(=O)c2ccc(NS(C)(=O)=O)cc2)c1]

Embodiment 33. The compound of any of claims 1-10, wherein B is

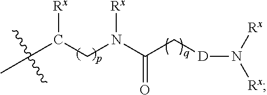

each R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups; p and q are independently chosen from 0, 1, 2, 3, and 4; C, D, and E are independently chosen from hydrogen, linear alkoxy groups, branched alkoxy groups, cyclic alkoxy groups, cycloalkyl groups, heterocycloalkyl groups, aryl groups, and heteroaryl groups.

Embodiment 34. The compound of claim 33, wherein B is chosen from

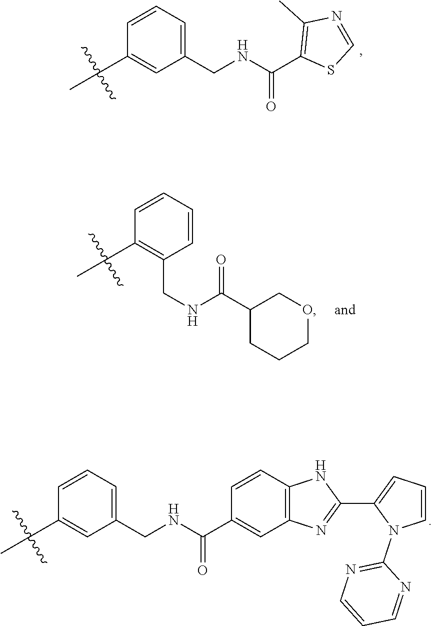 [SMILES: *c1cccc(CNC(=O)c2scnc2C)c1 ; *c1ccccc1CNC(=O)C1CCCOC1 ; *c1cccc(CNC(=O)c2ccc3[nH]c(-c4cccn4-c4ncccn4)nc3c2)c1]

Embodiment 35. The compound of any of claims 1-10, wherein B is

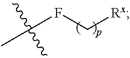

R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups; and F is chosen from linear alkoxy groups, branched alkoxy groups, cyclic alkoxy groups, cycloalkyl groups, heterocycloalkyl groups, aryl groups, and heteroaryl groups.

Embodiment 36. The compound of claim 33, wherein B is

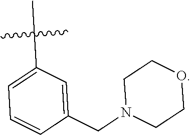 [SMILES: *c1cccc(CN2CCOCC2)c1]

Embodiment 37. The compound of any of the preceding claims, wherein one of X⁷ and X⁸ is chosen from hydrogen, amino groups, —NHC(O)alkylgroups, —NHC(O)arylalkylgroups, and —NHC(O)heteroarylalkyl groups.

Embodiment 38. The compound of any of the preceding claims, wherein one of X¹ and X^{2 from —NH}2, —NHC(O)CH₃, and

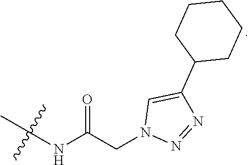 [SMILES: *NC(=O)Cn1cc(C2CCCCC2)nn1]

Embodiment 39. The compound of any of one of claims 1-29, wherein Z is hydrogen.

Embodiment 40. The compound of any of one of claims 1-29, wherein Z is —CN.

Embodiment 41. The compound of any of one of claims 1-29, wherein Z is —CO₂H.

Embodiment 42. The compound of any of one of claims 1-29, wherein Z is —C(O)R^{z}, —CO₂R^{z}, or —C(O)NHSO₂R^{z}; wherein R is chosen from hydrogen, linear alkyl groups, branched alkyl groups, cyclic alkyl groups, carbocyclic groups, amino groups, heterocyclic groups, aryl groups, and heteroaryl groups.

Embodiment 43. The compound of any of one of claims 1-29, wherein Z is —C(O)NHCN.

Embodiment 44. The compound of claim 1, wherein the compound is chosen from:

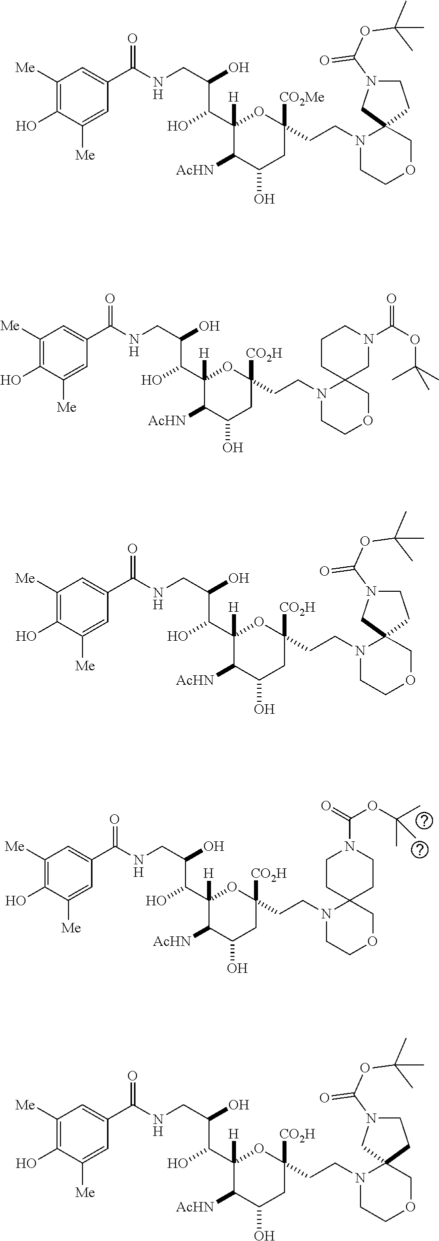

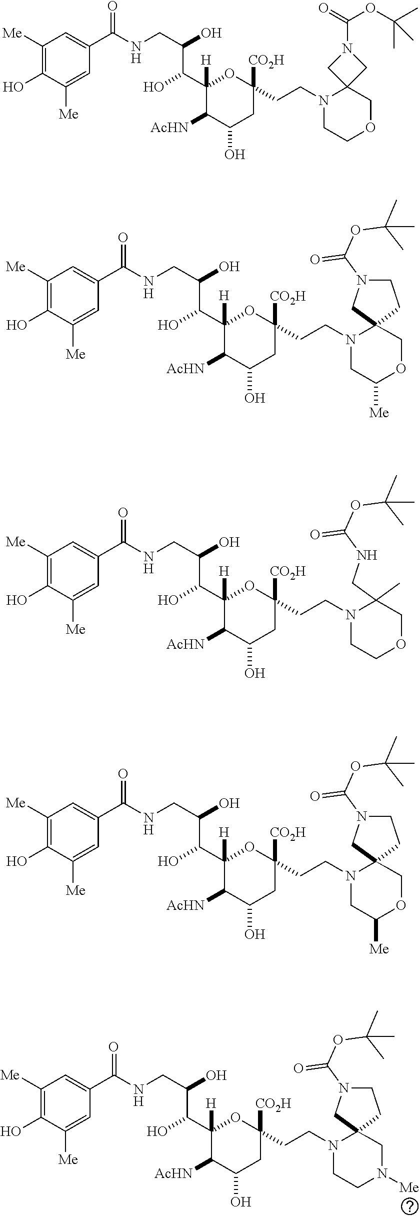

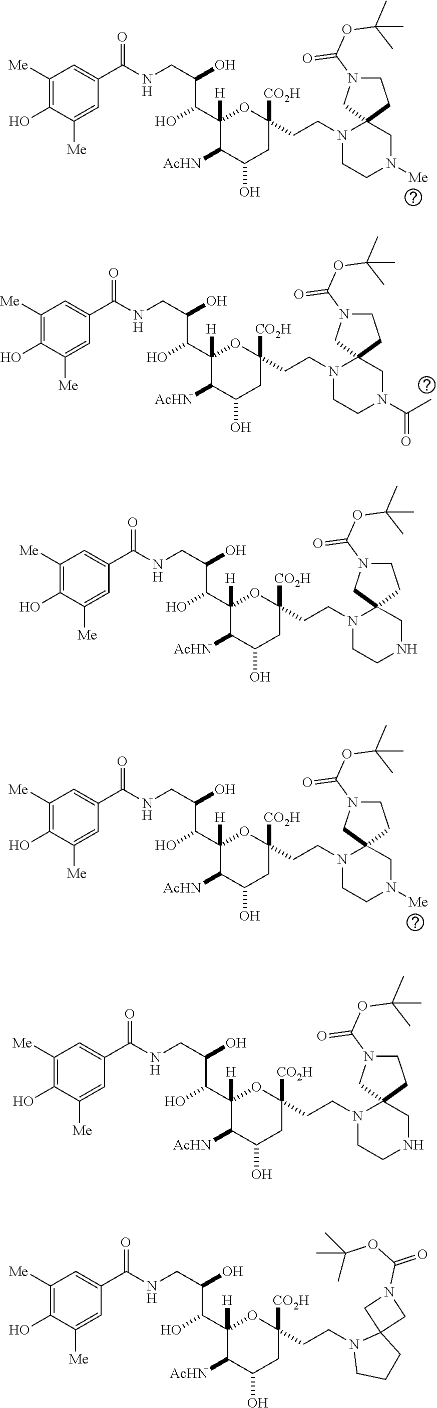

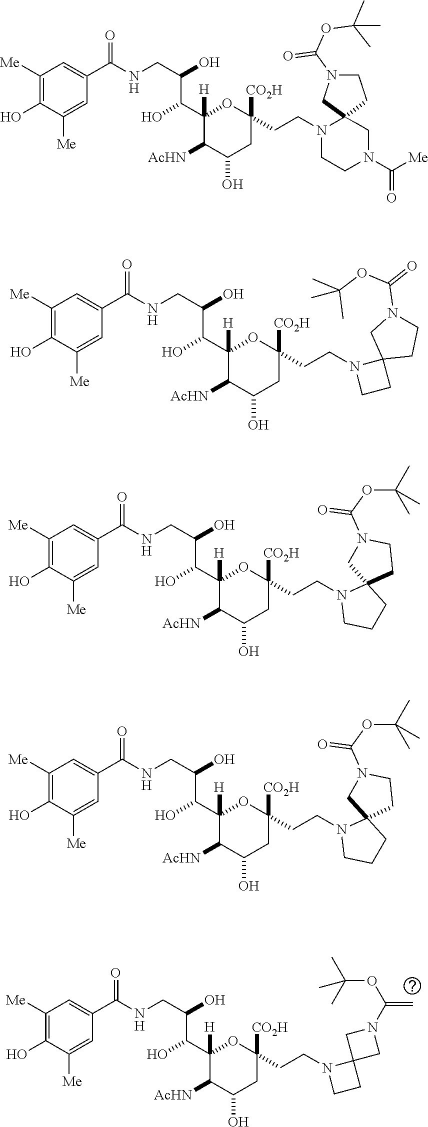

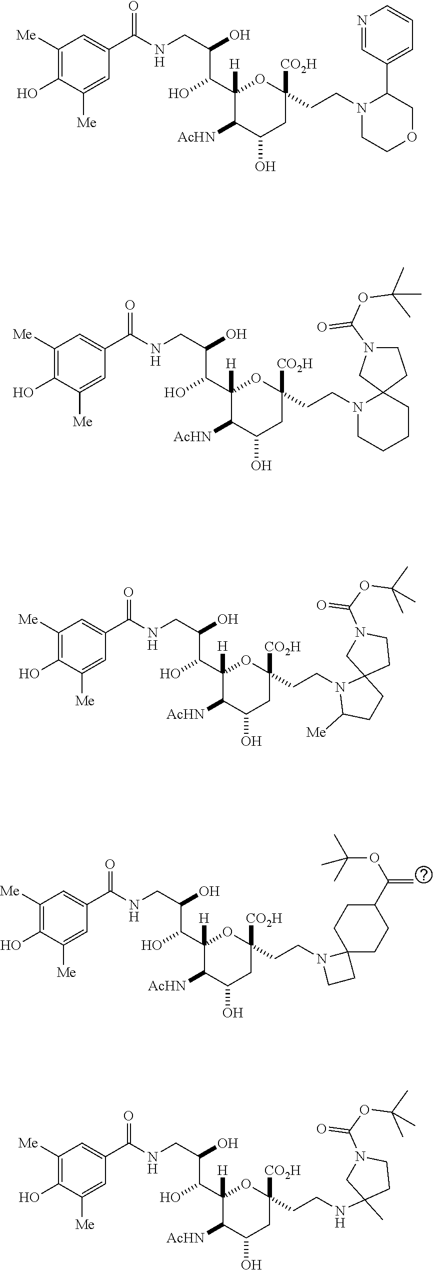

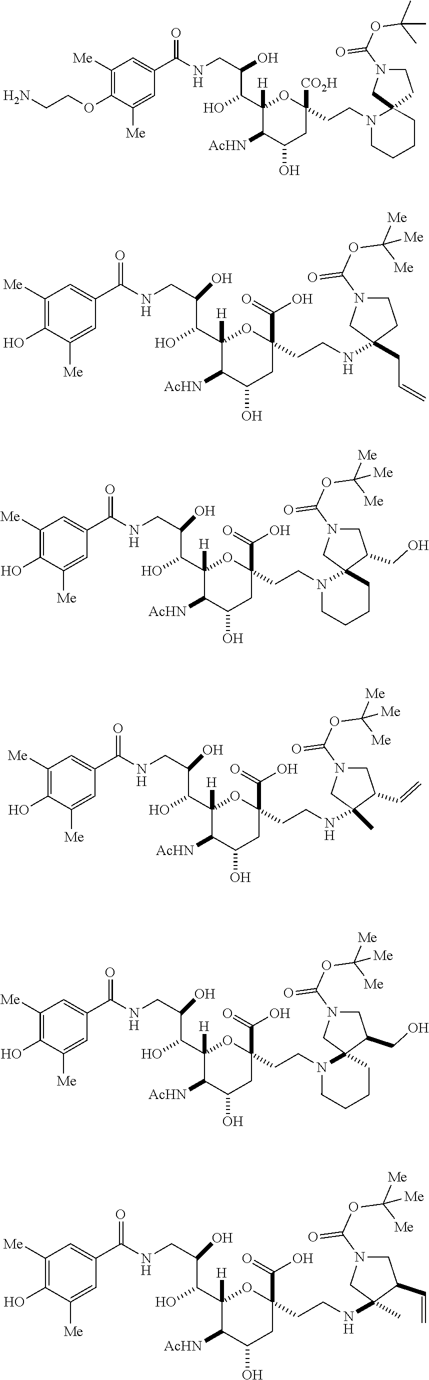

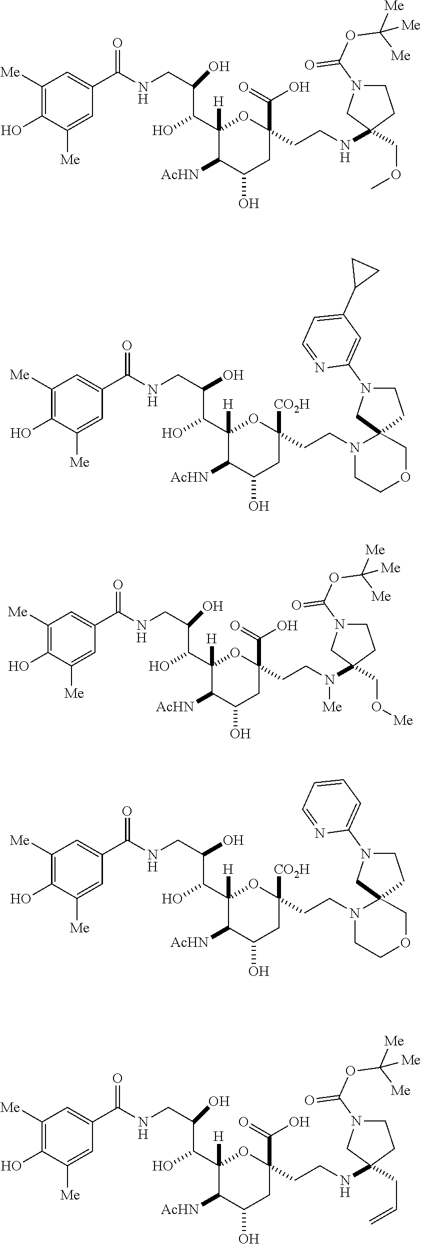

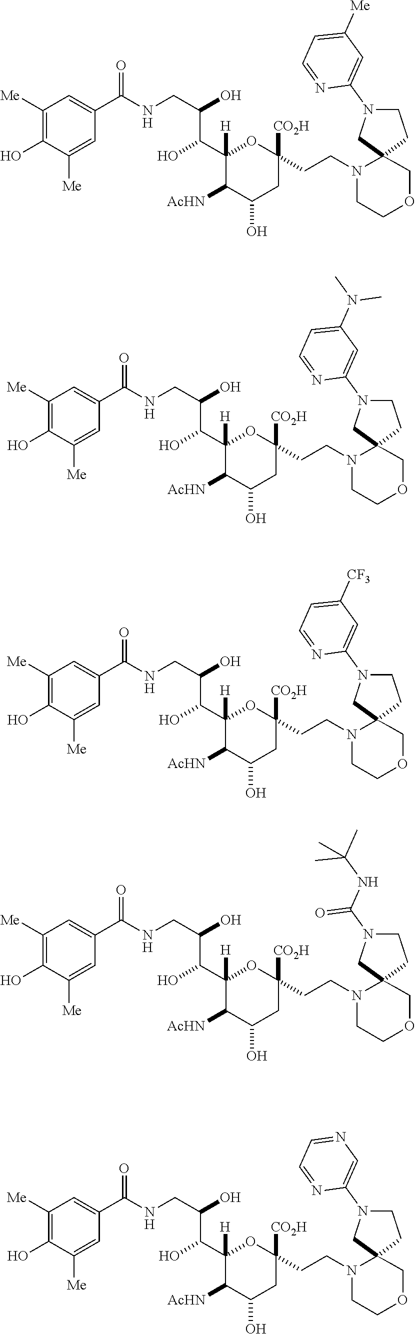

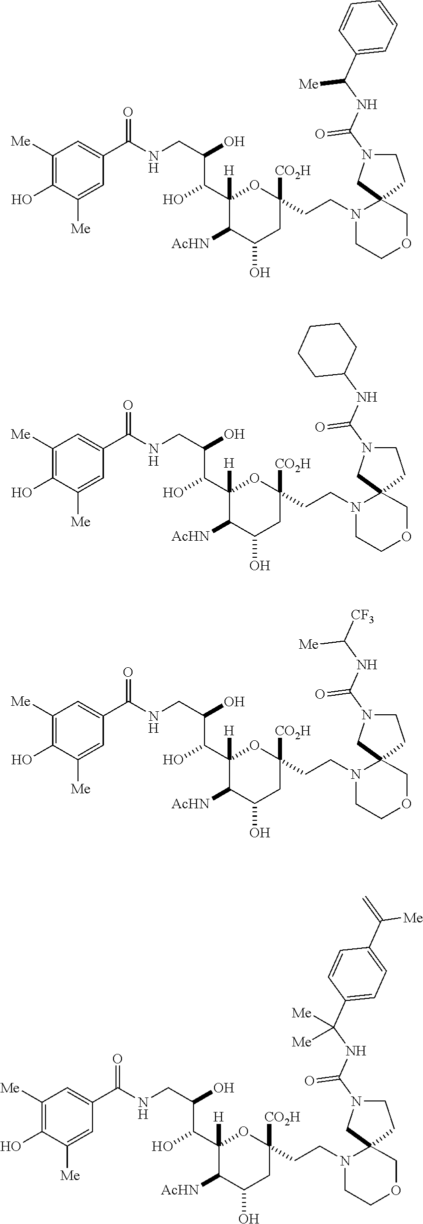

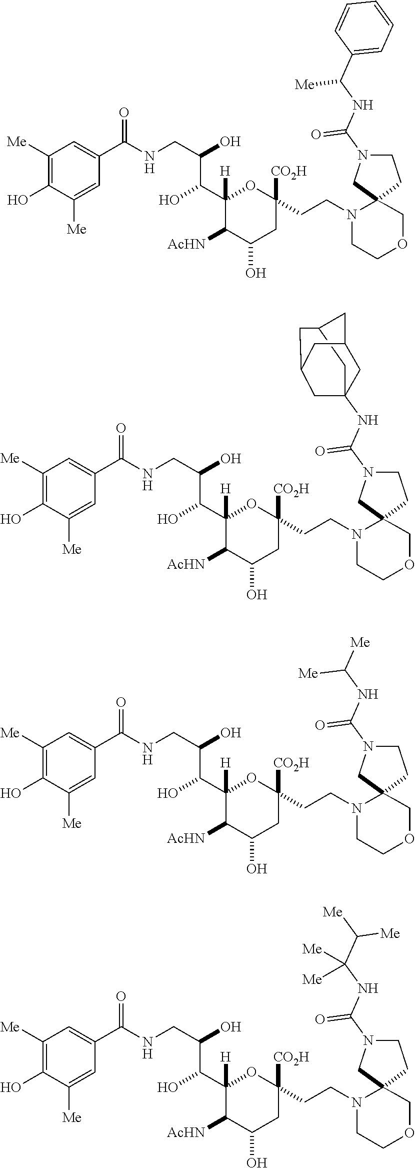

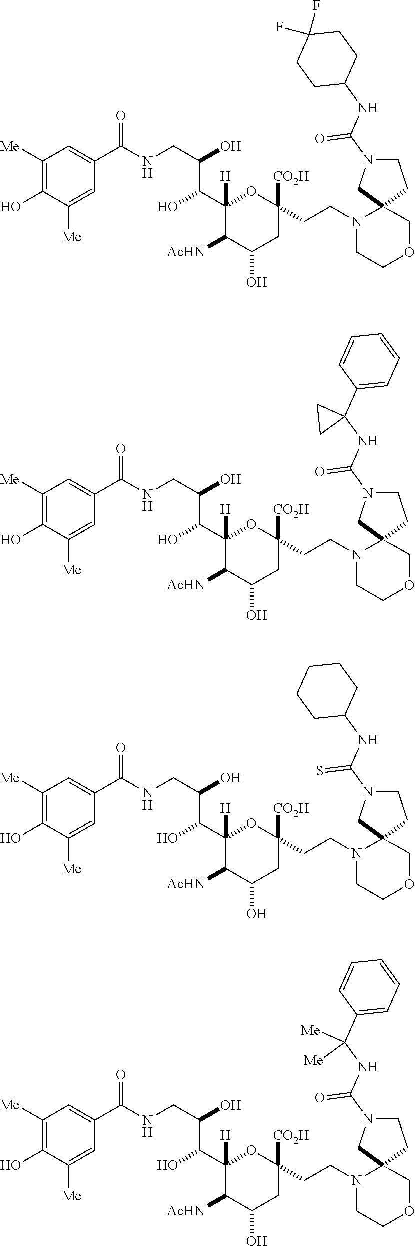

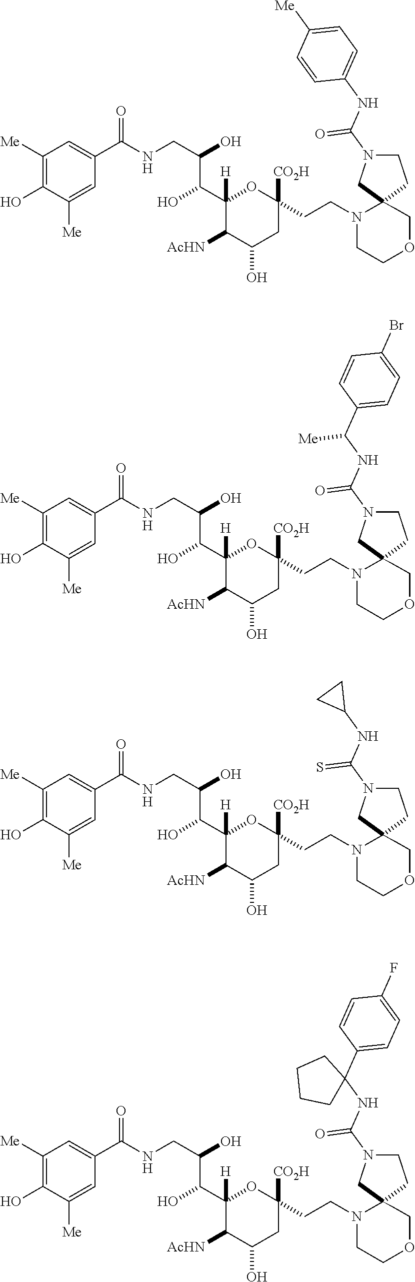

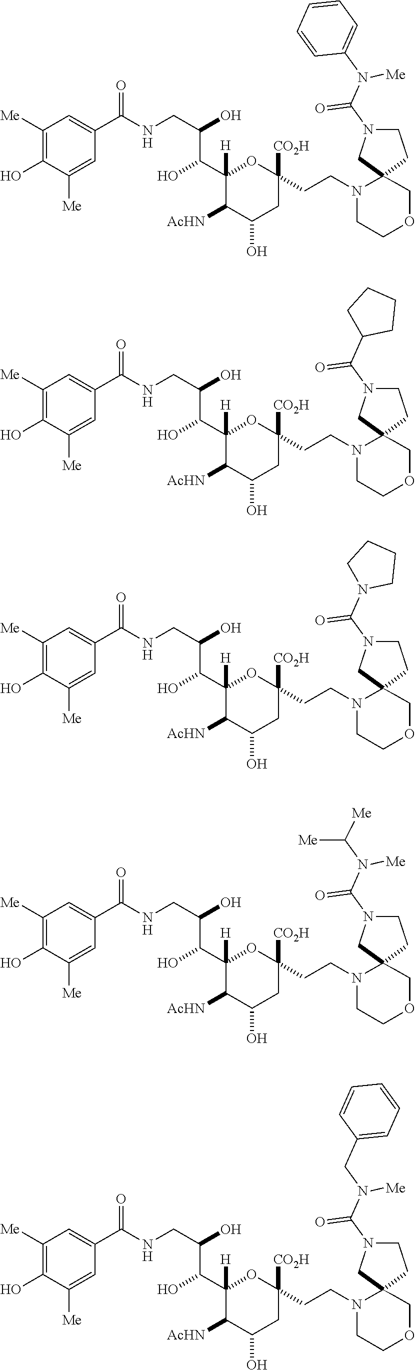

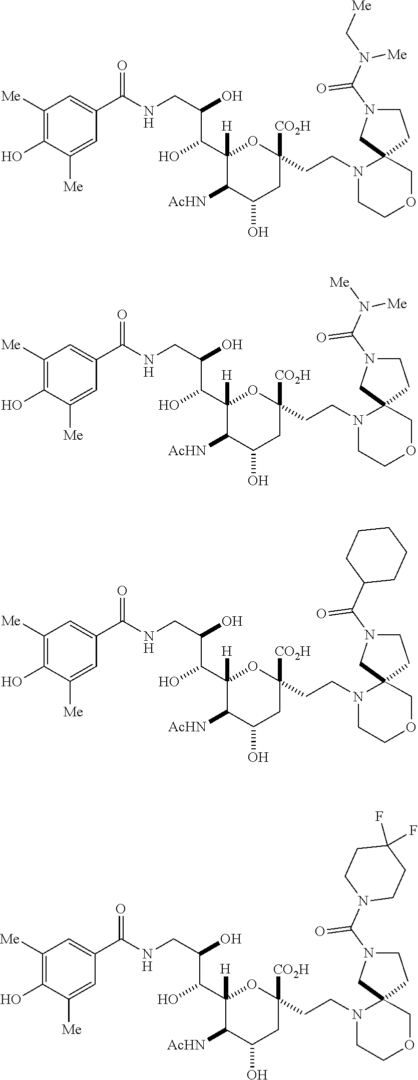

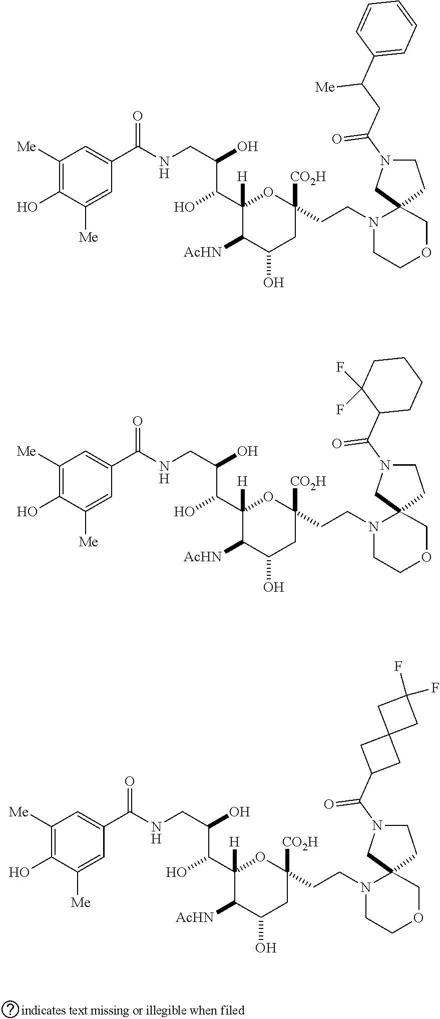

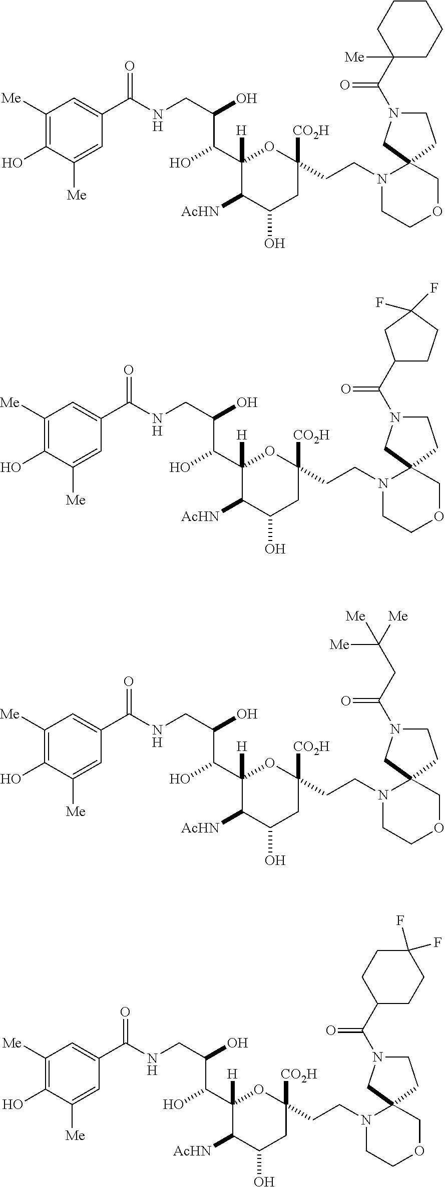

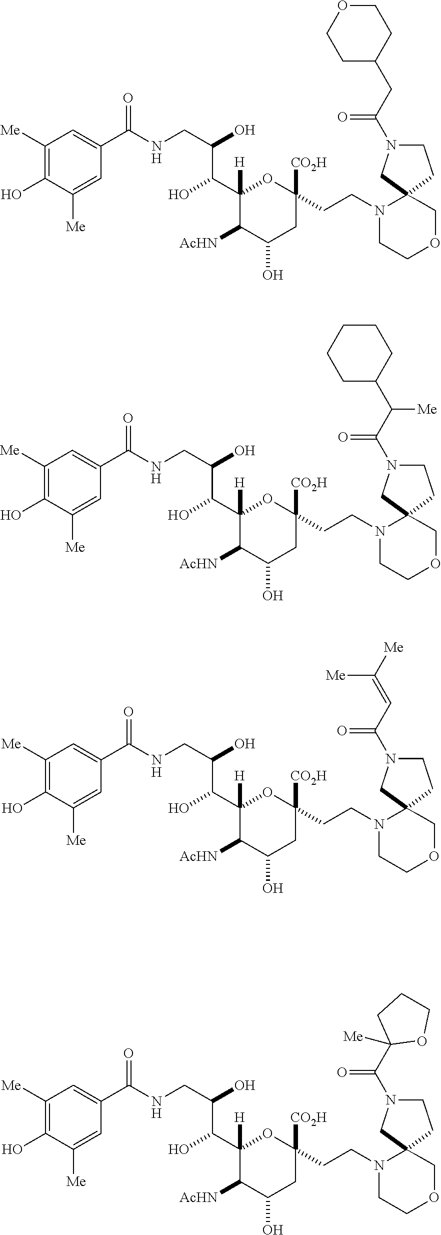

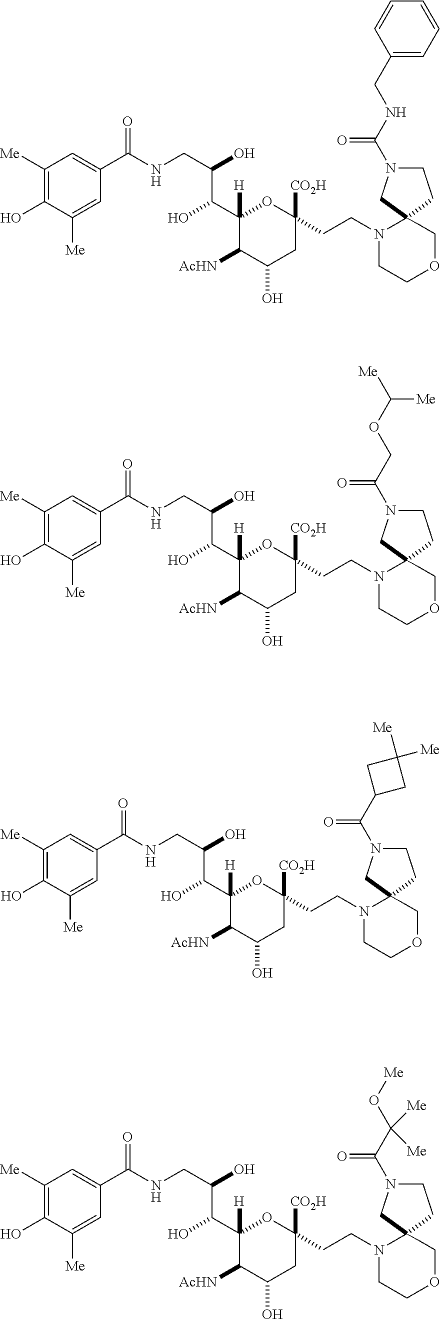

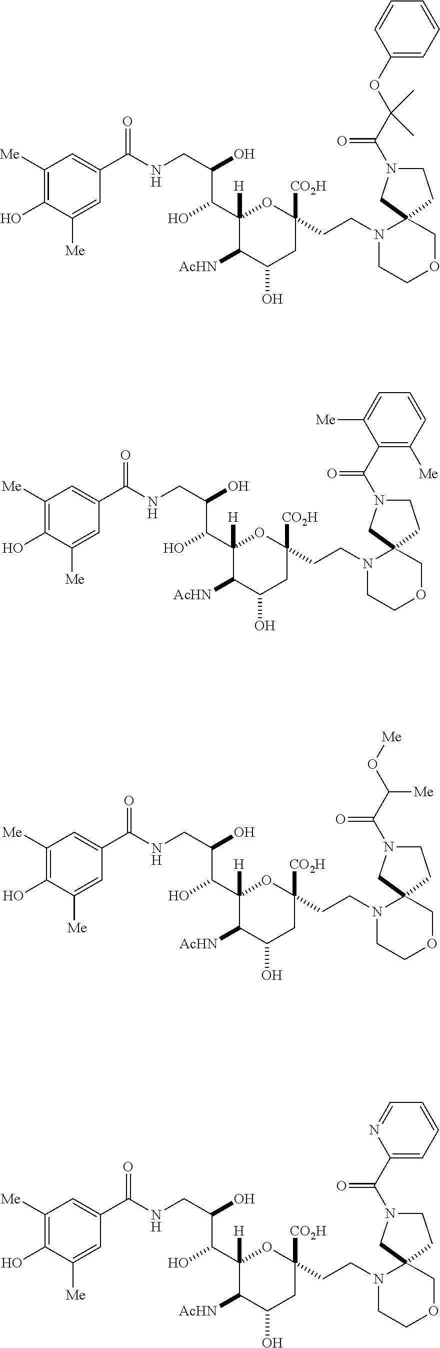

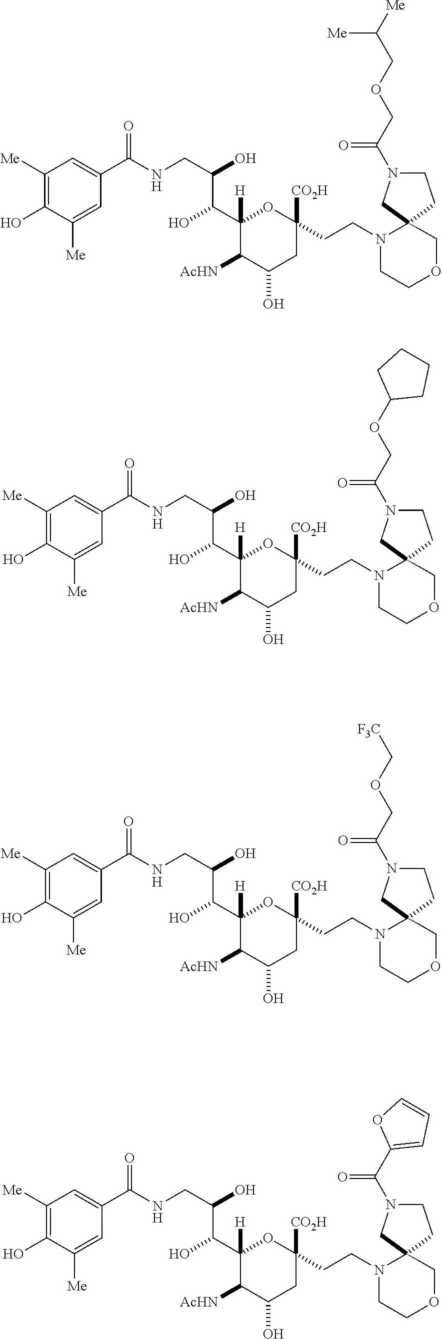

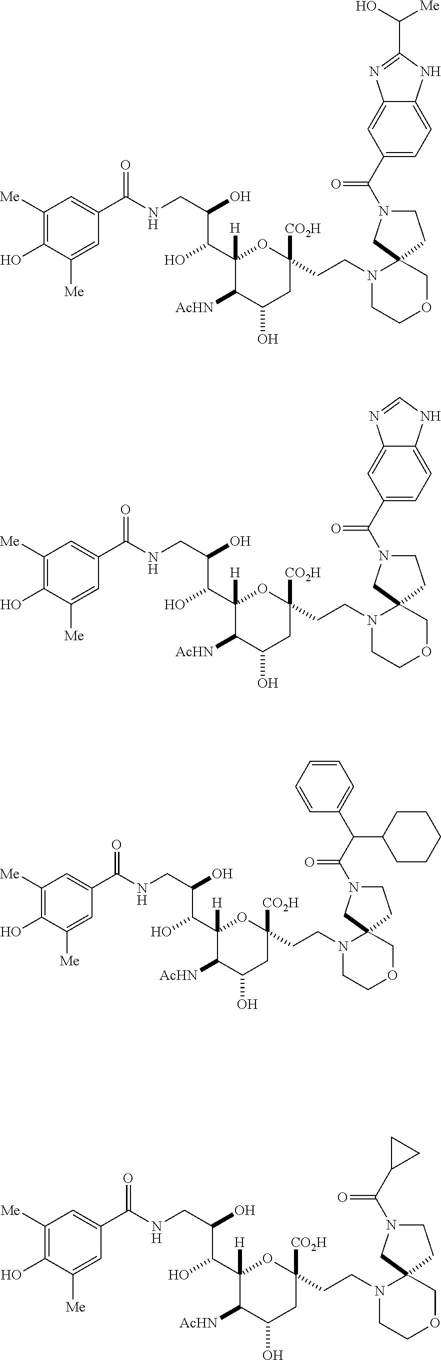

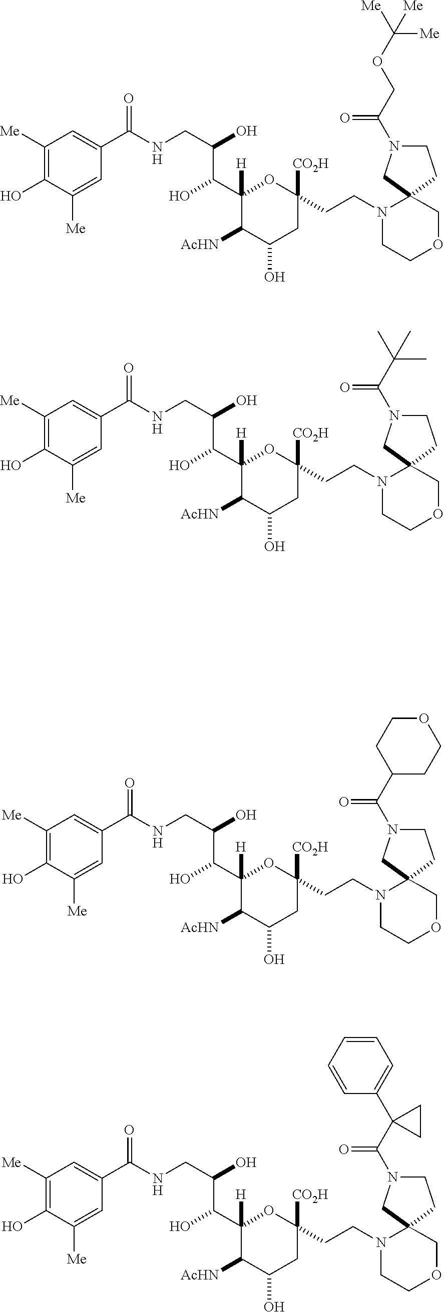

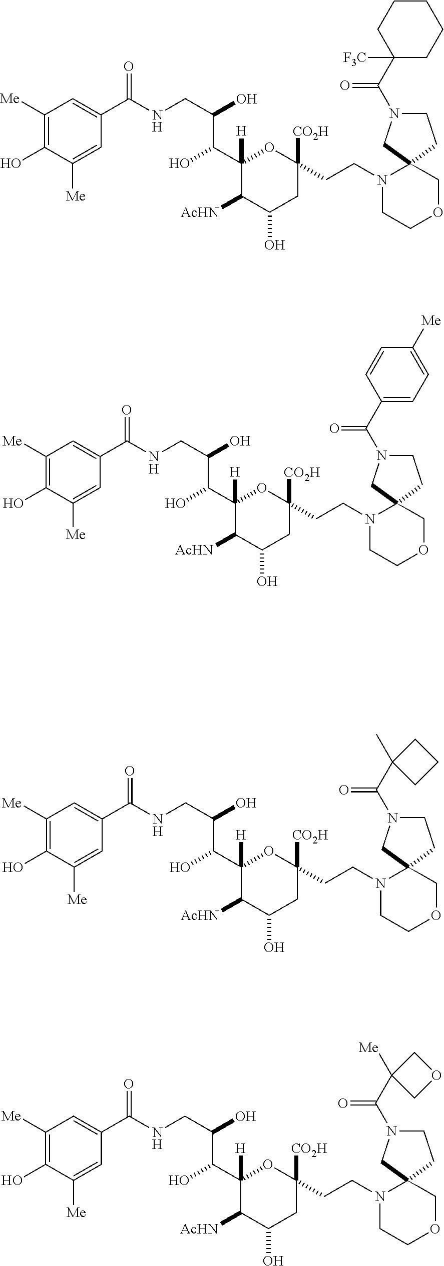

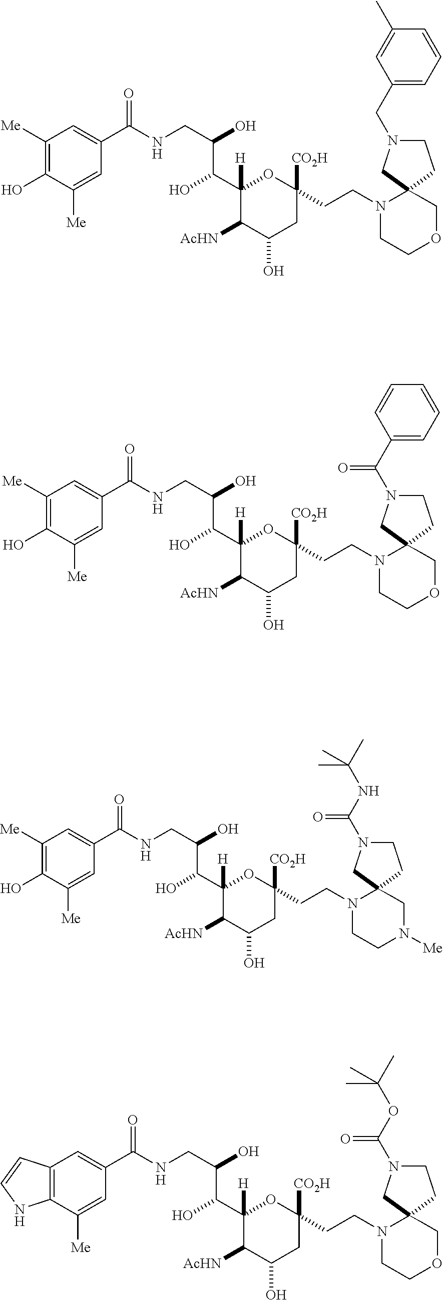

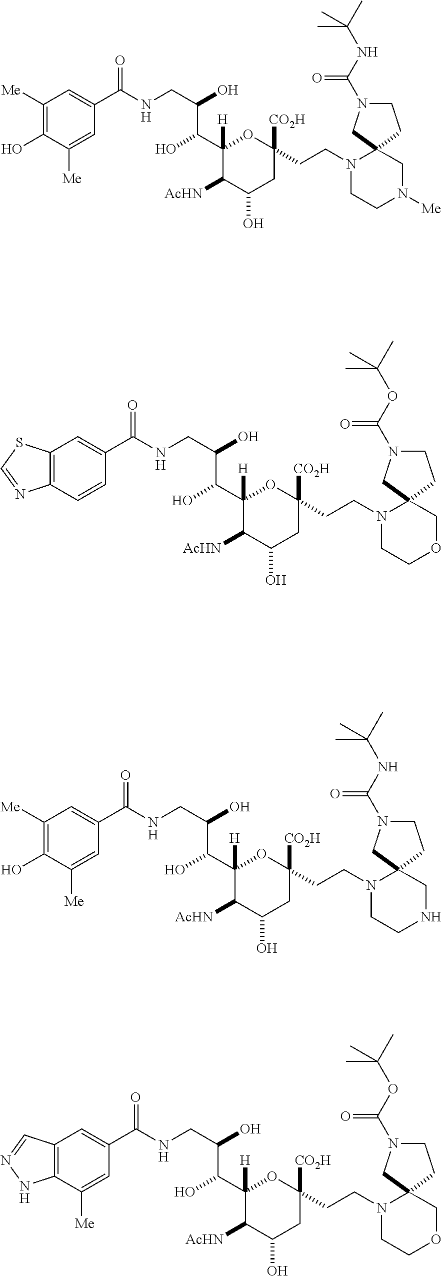

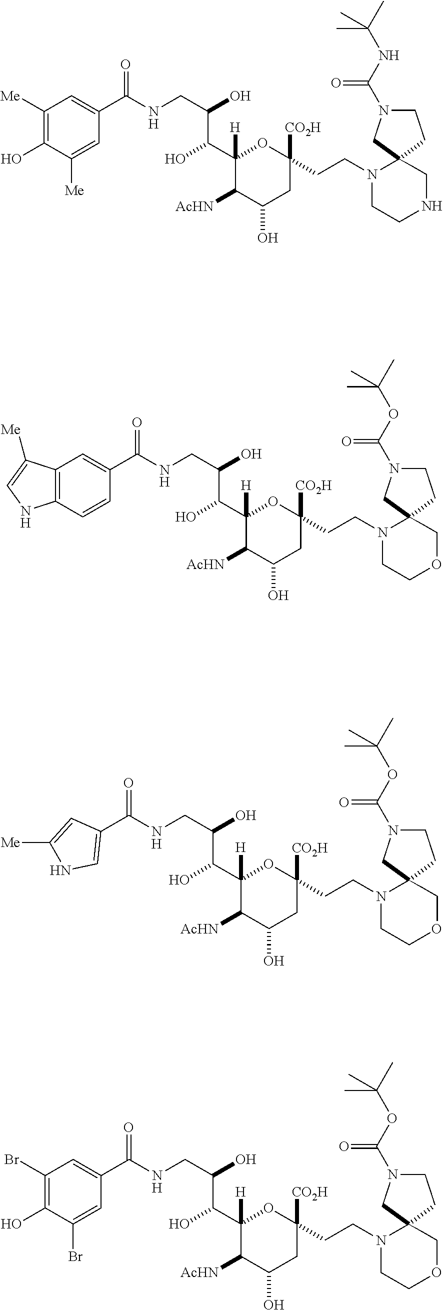

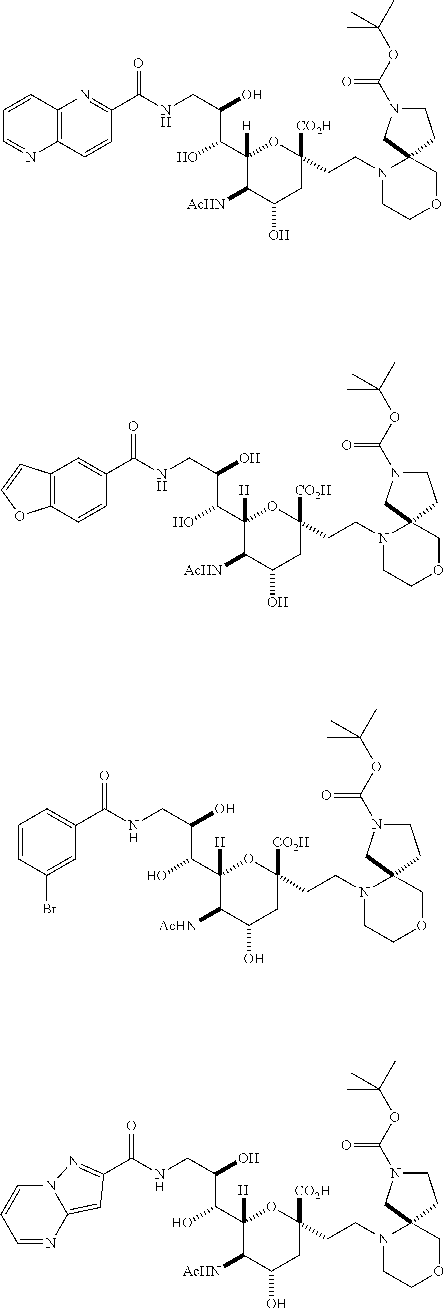

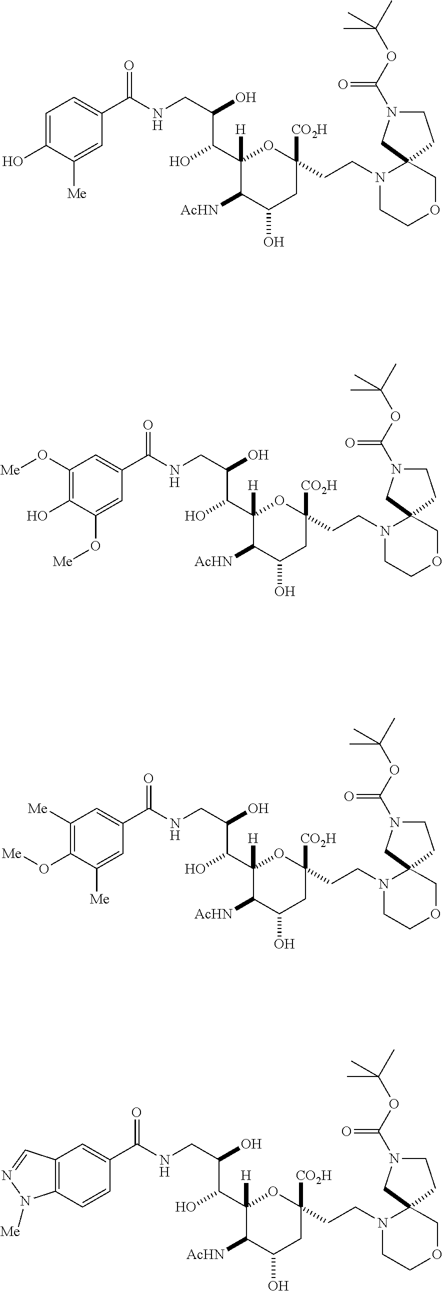

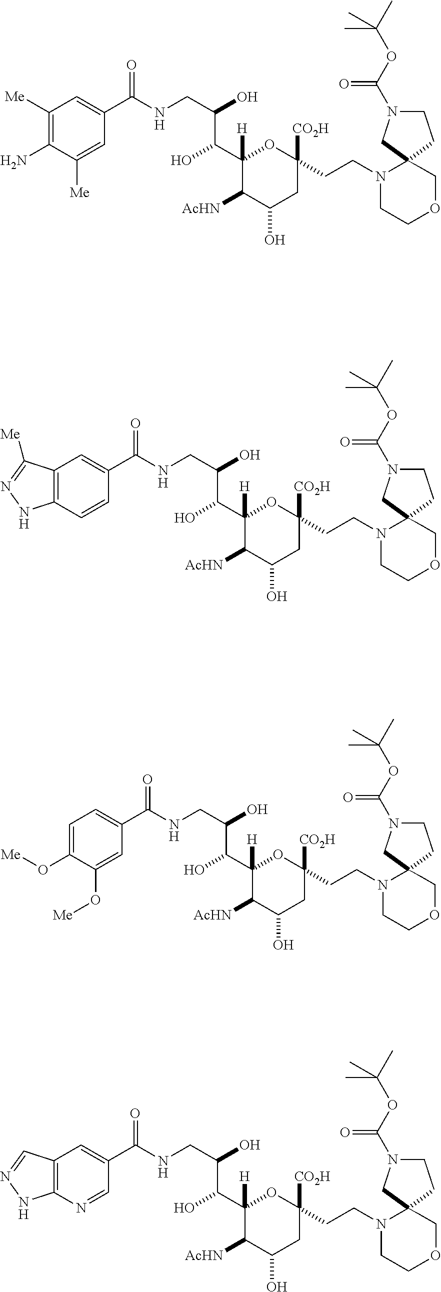

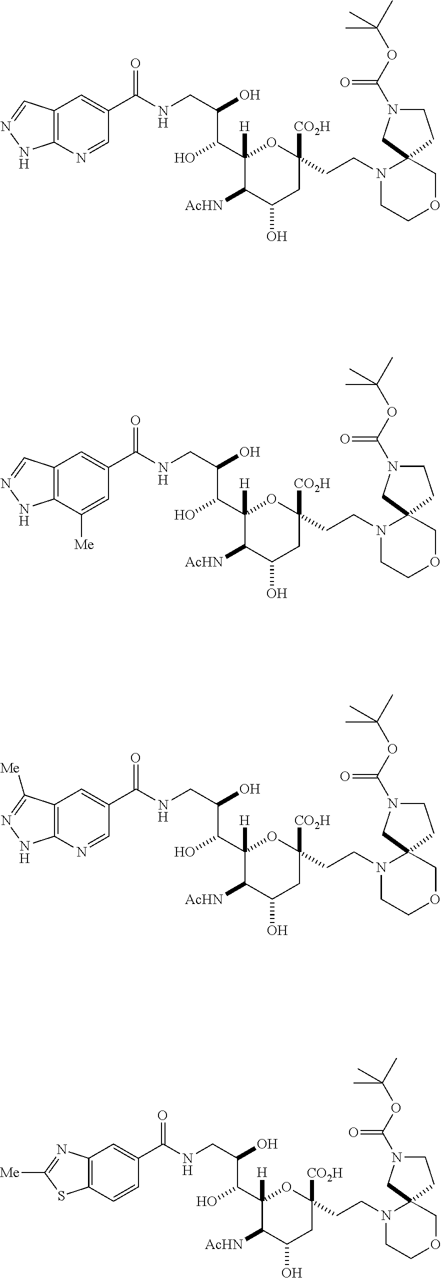

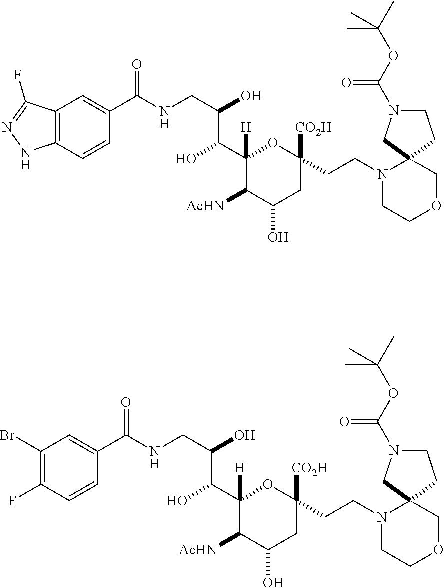

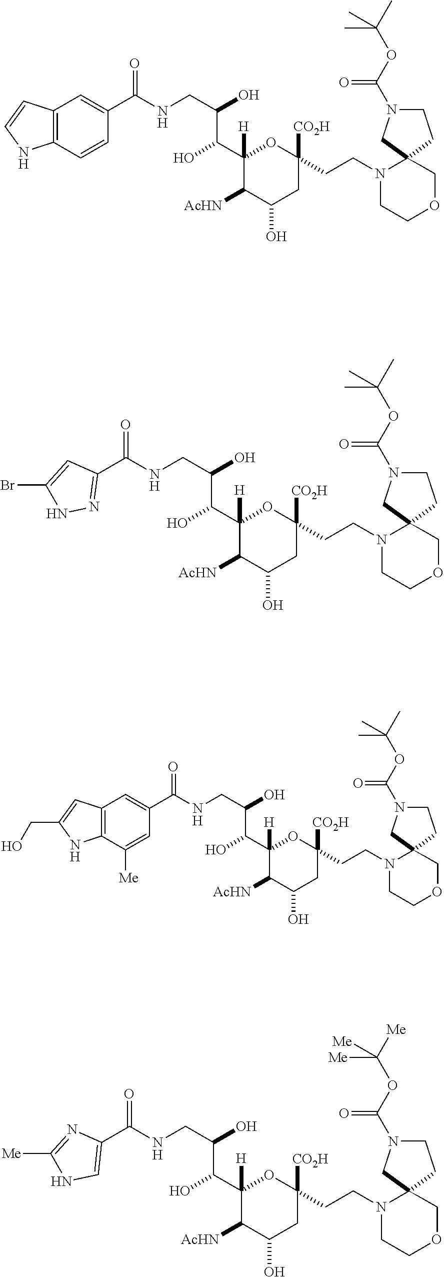

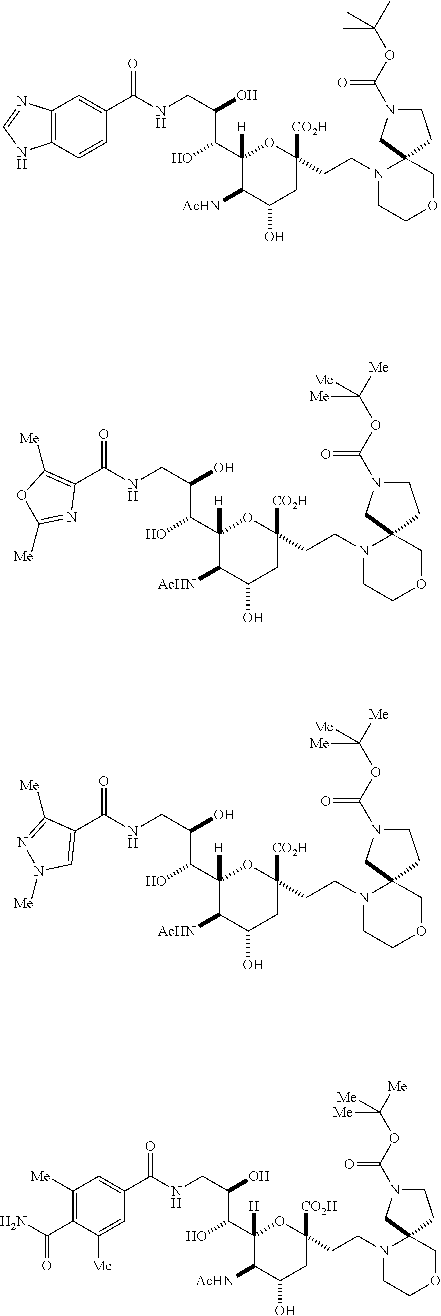

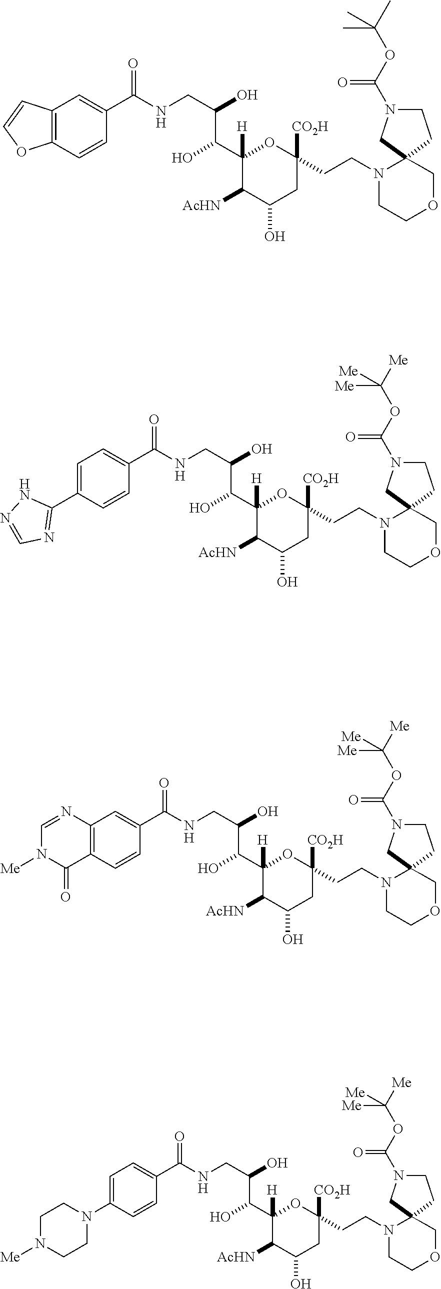

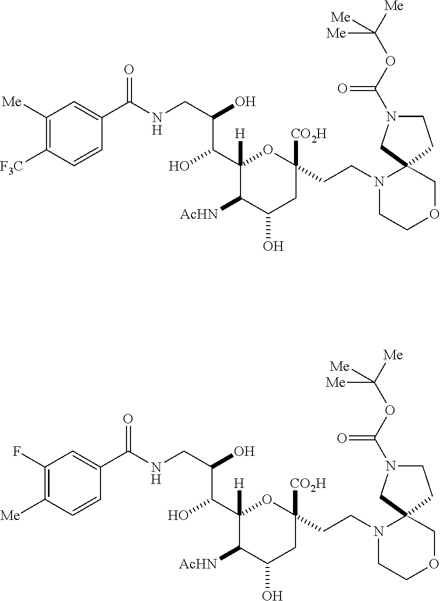

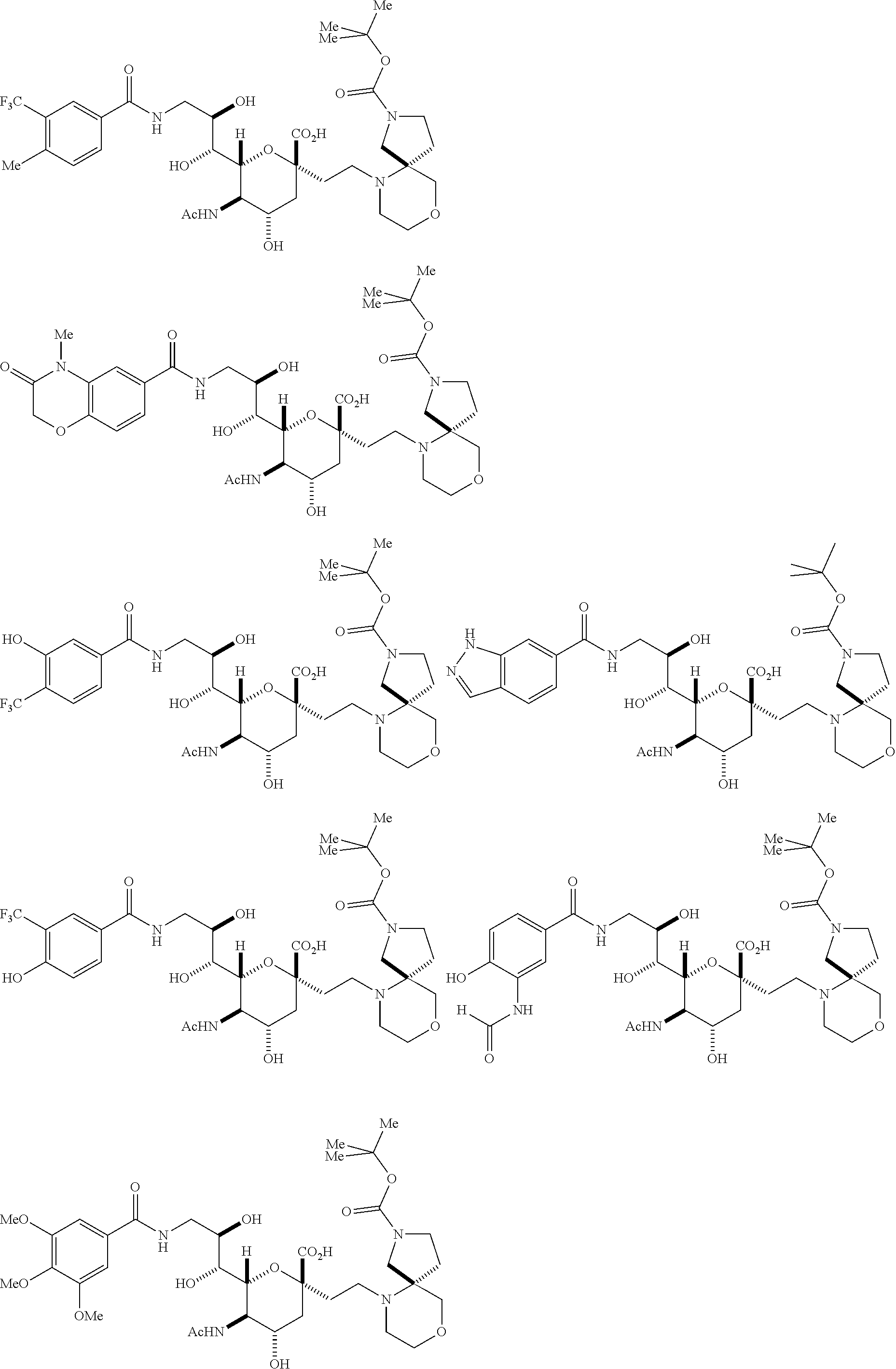

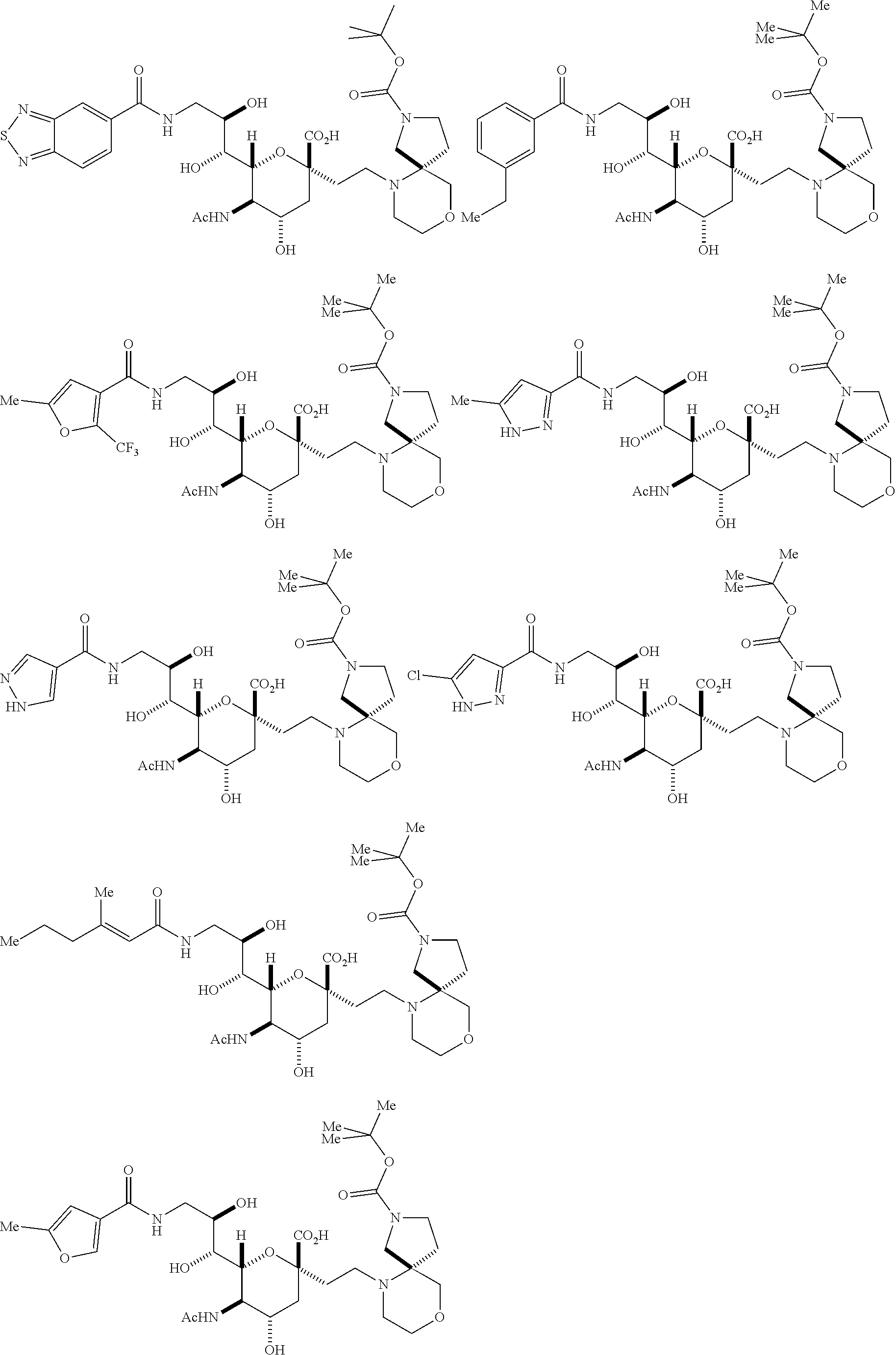

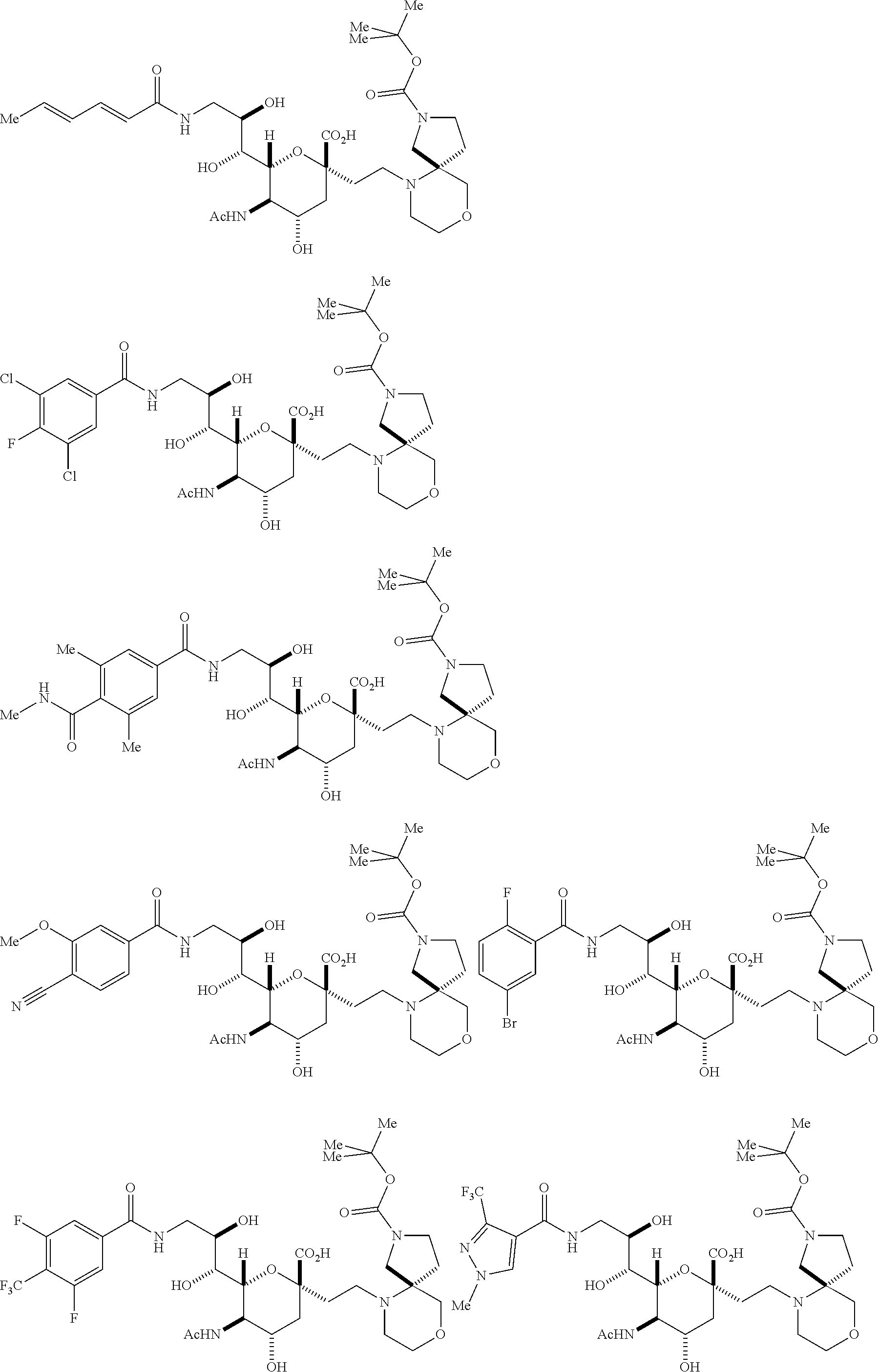

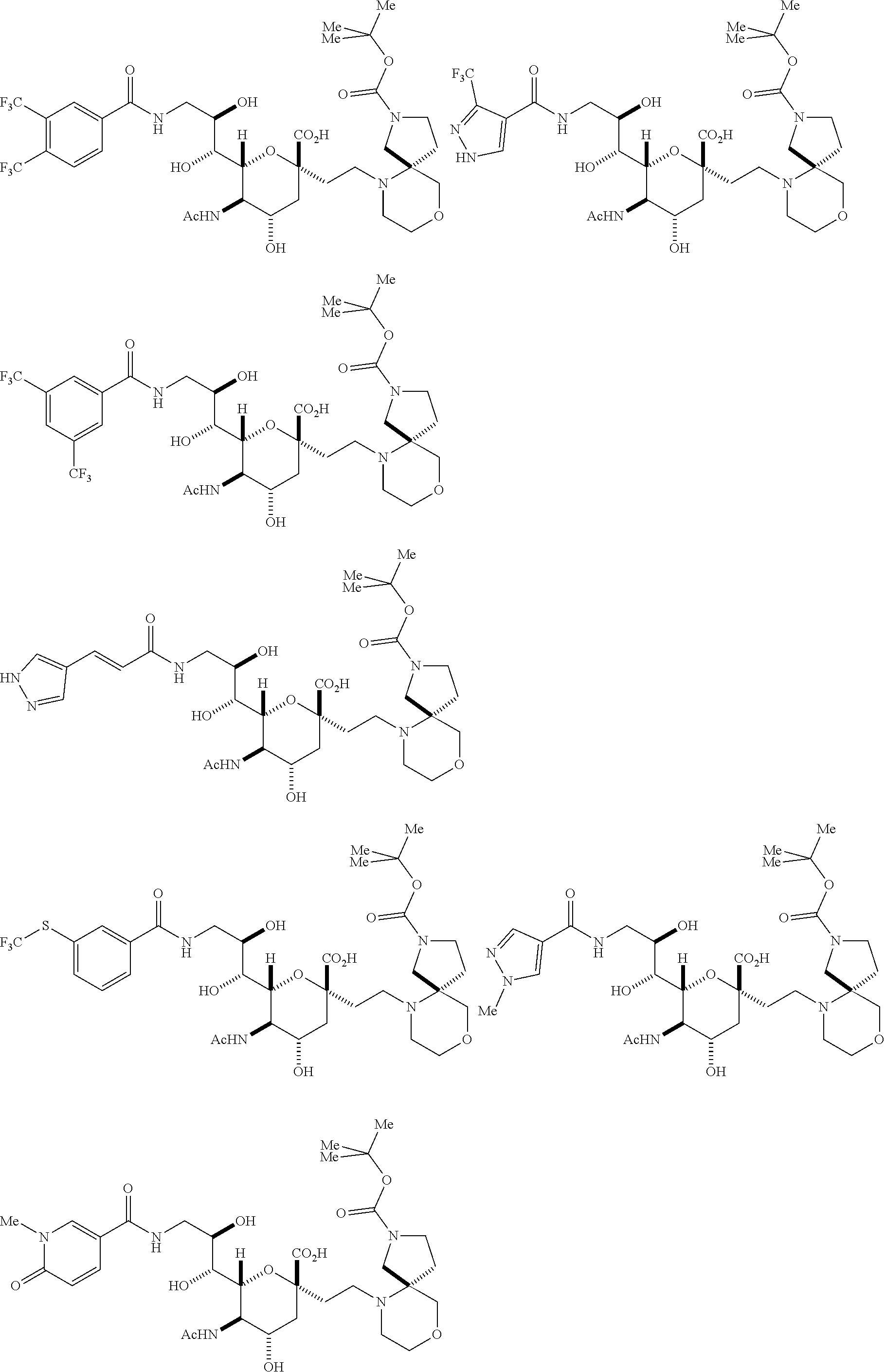

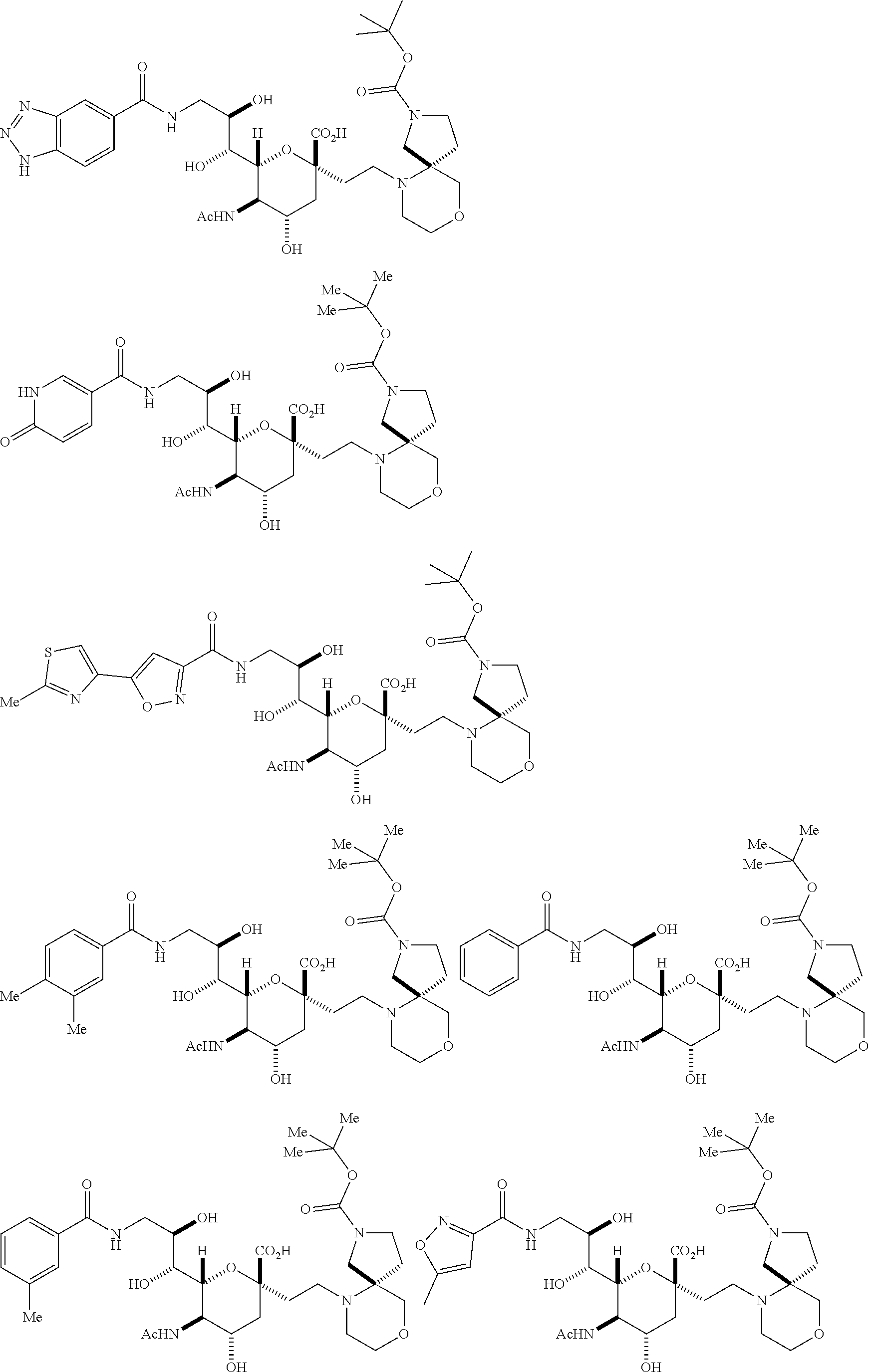

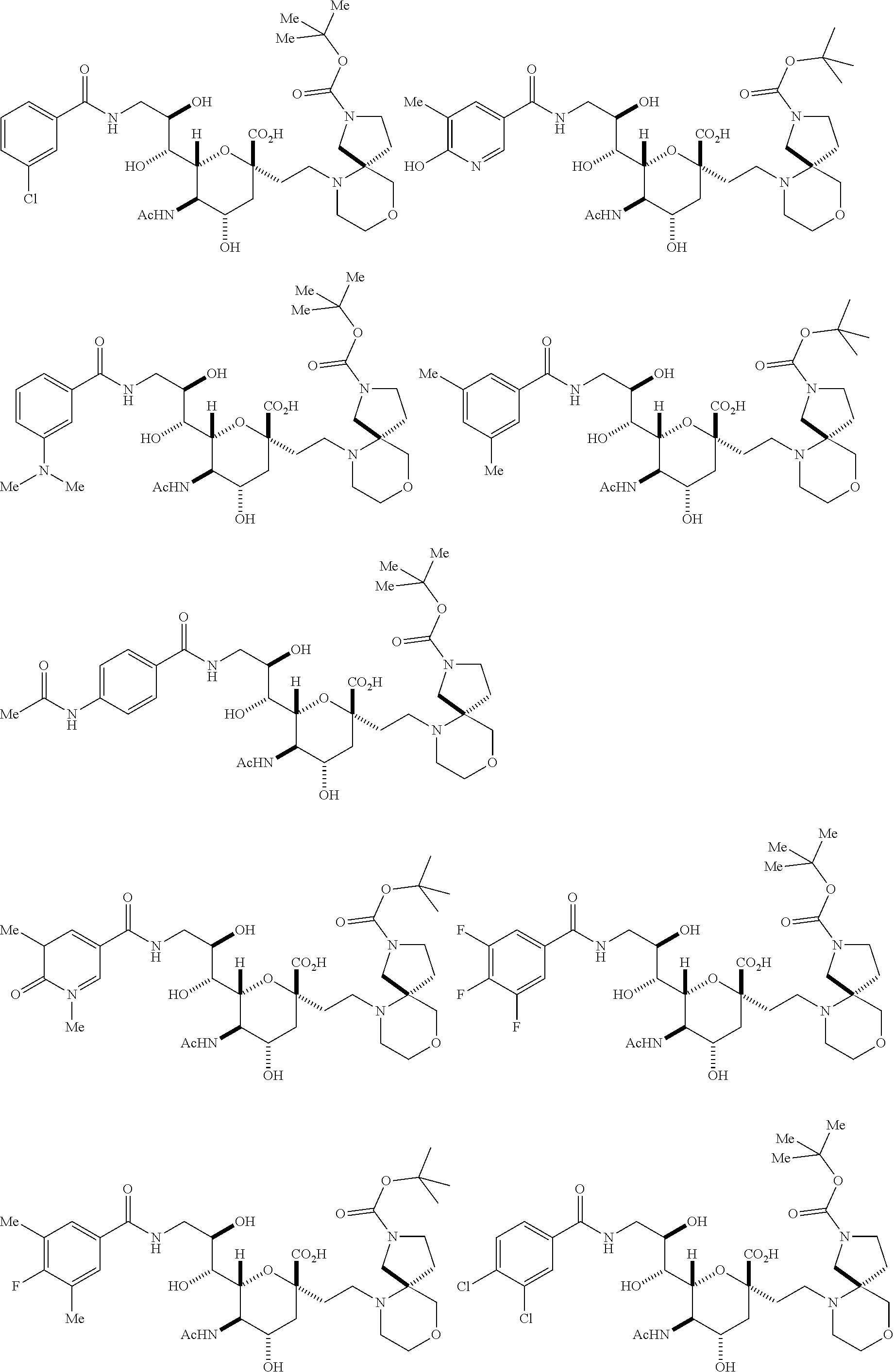

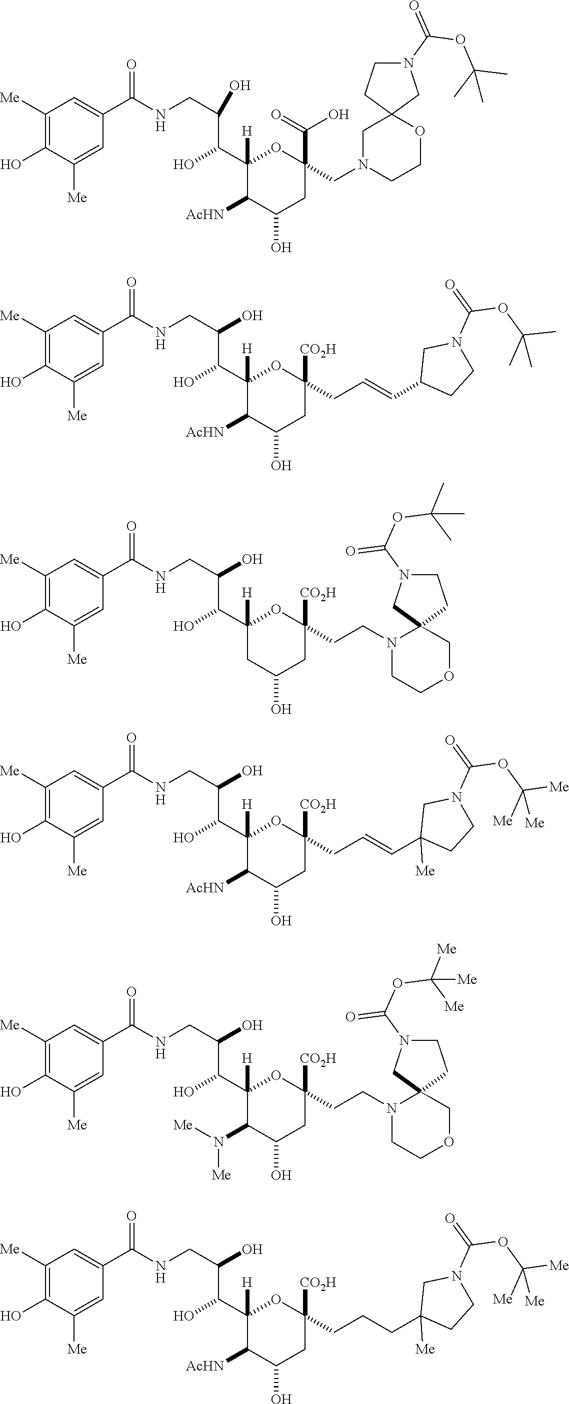

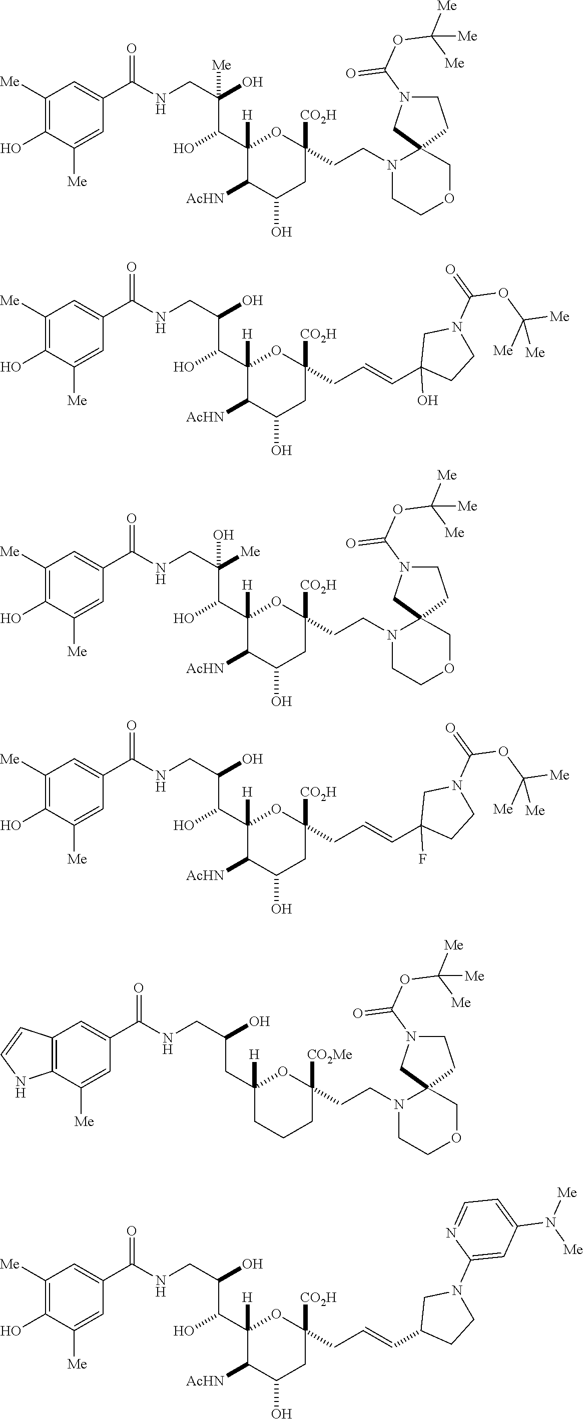

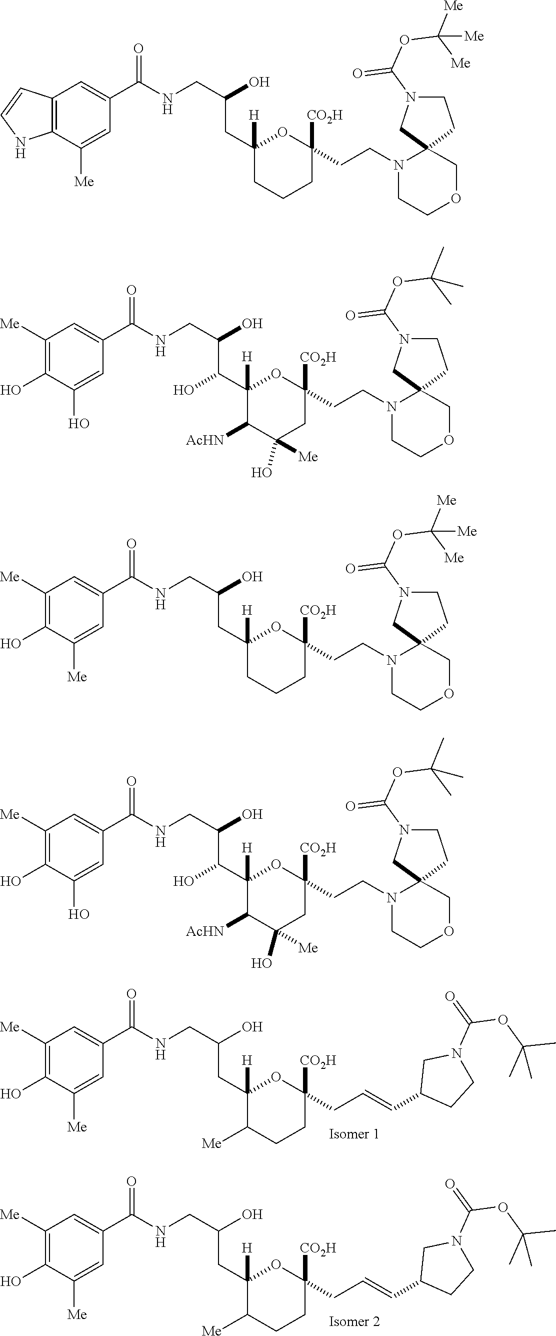

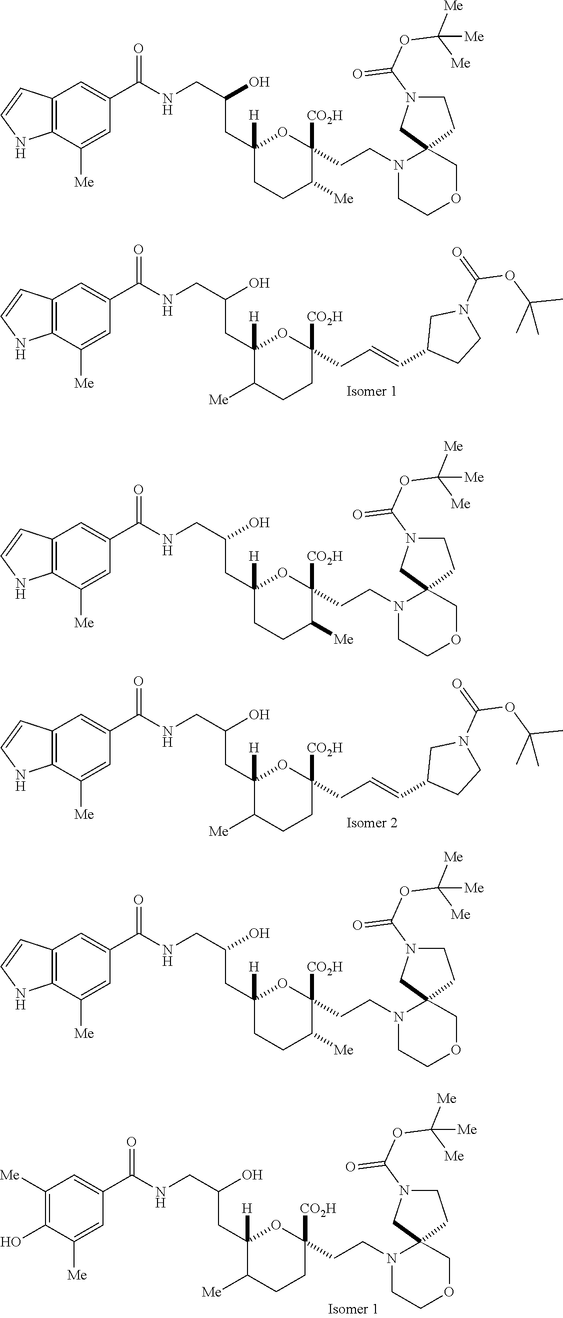

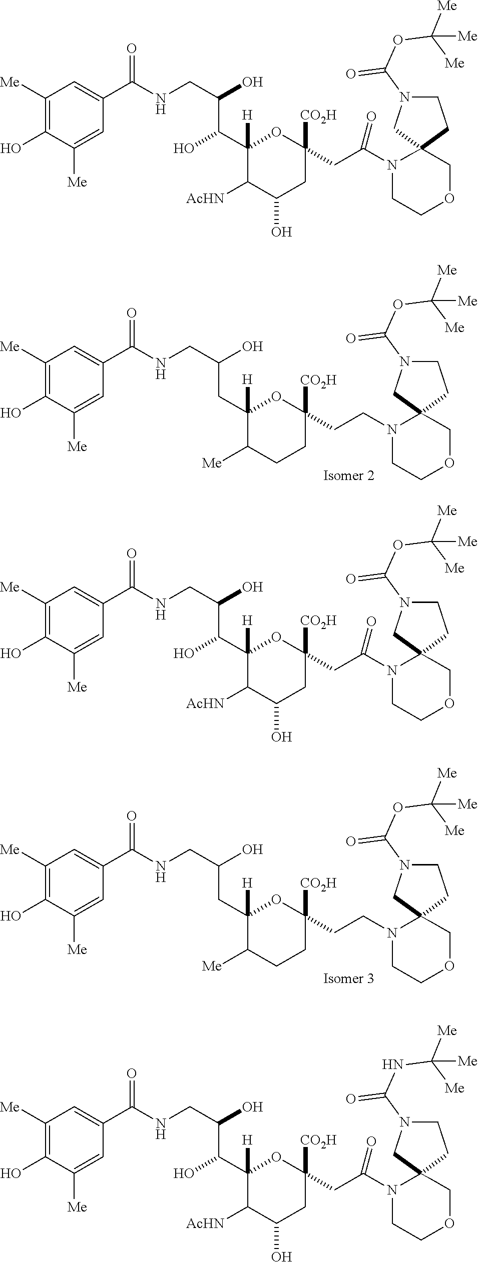

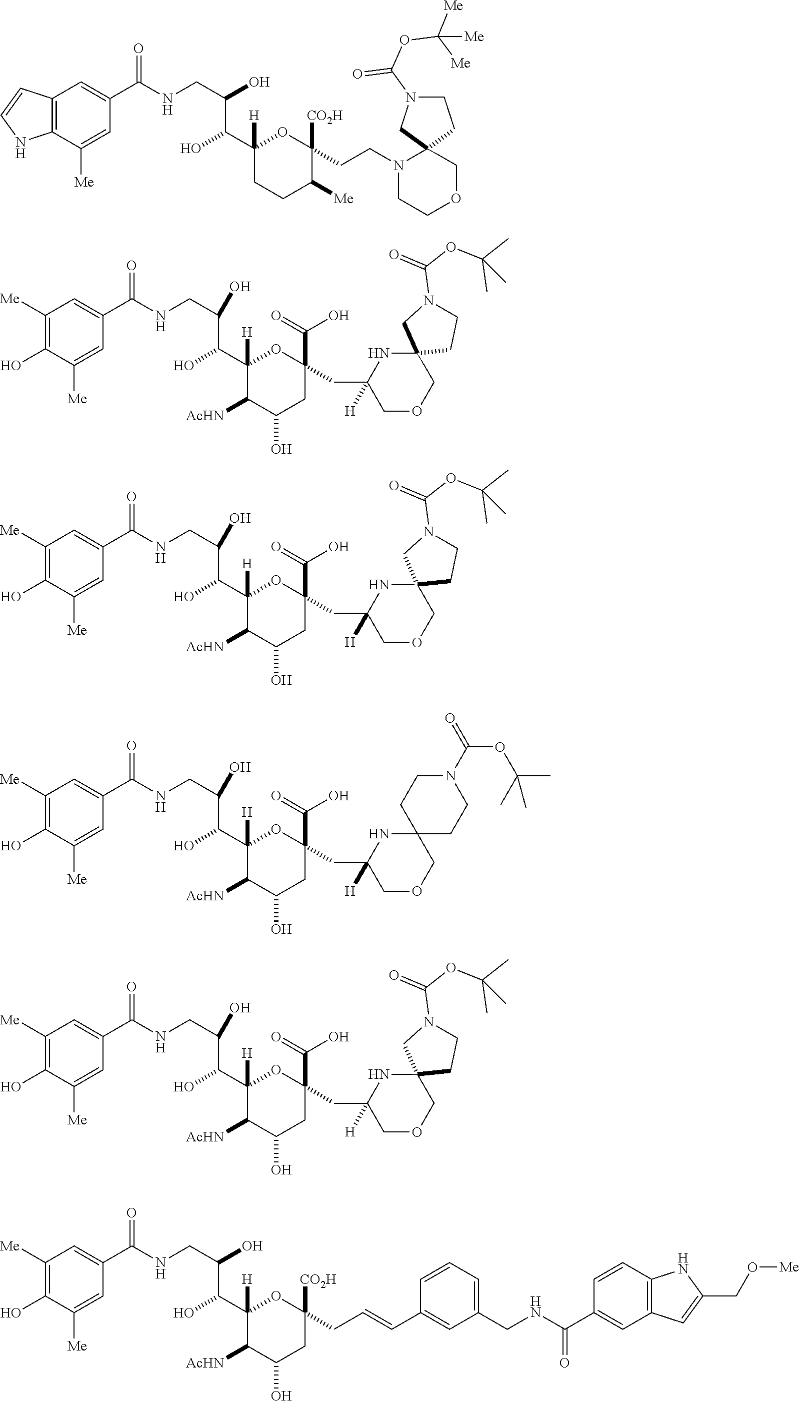

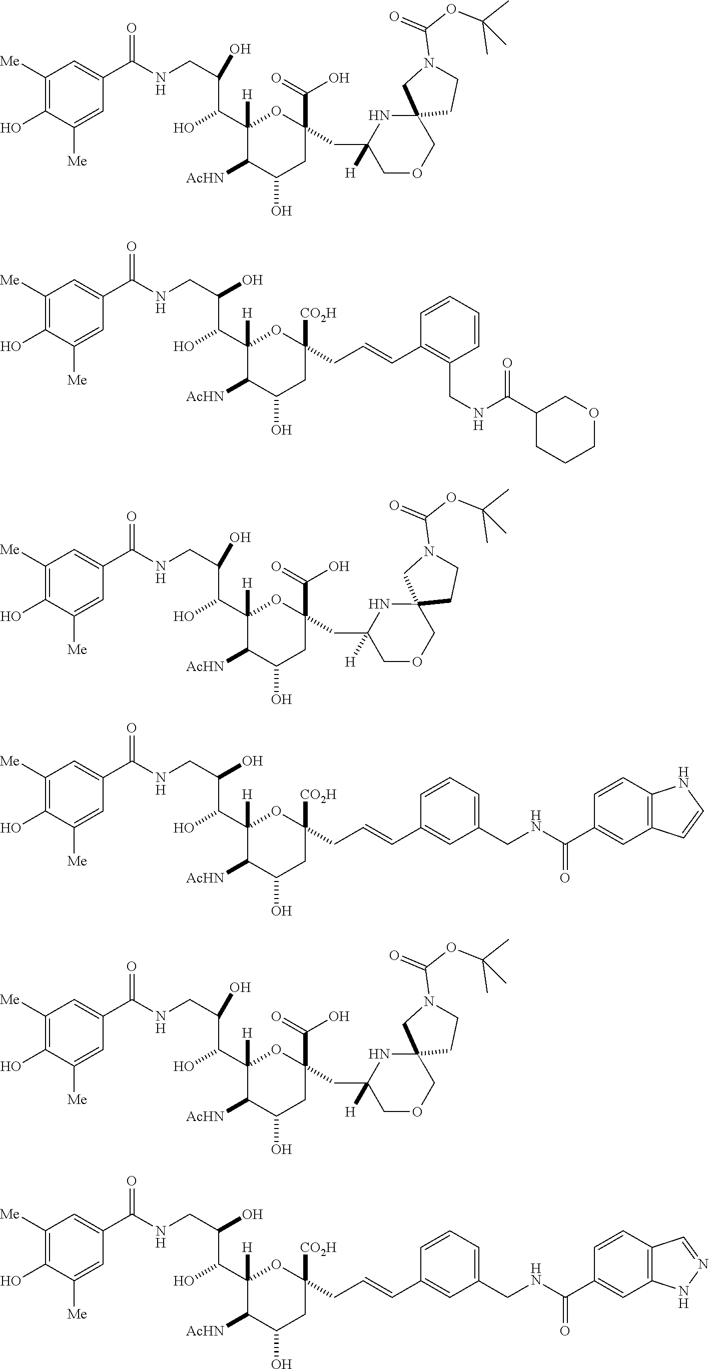

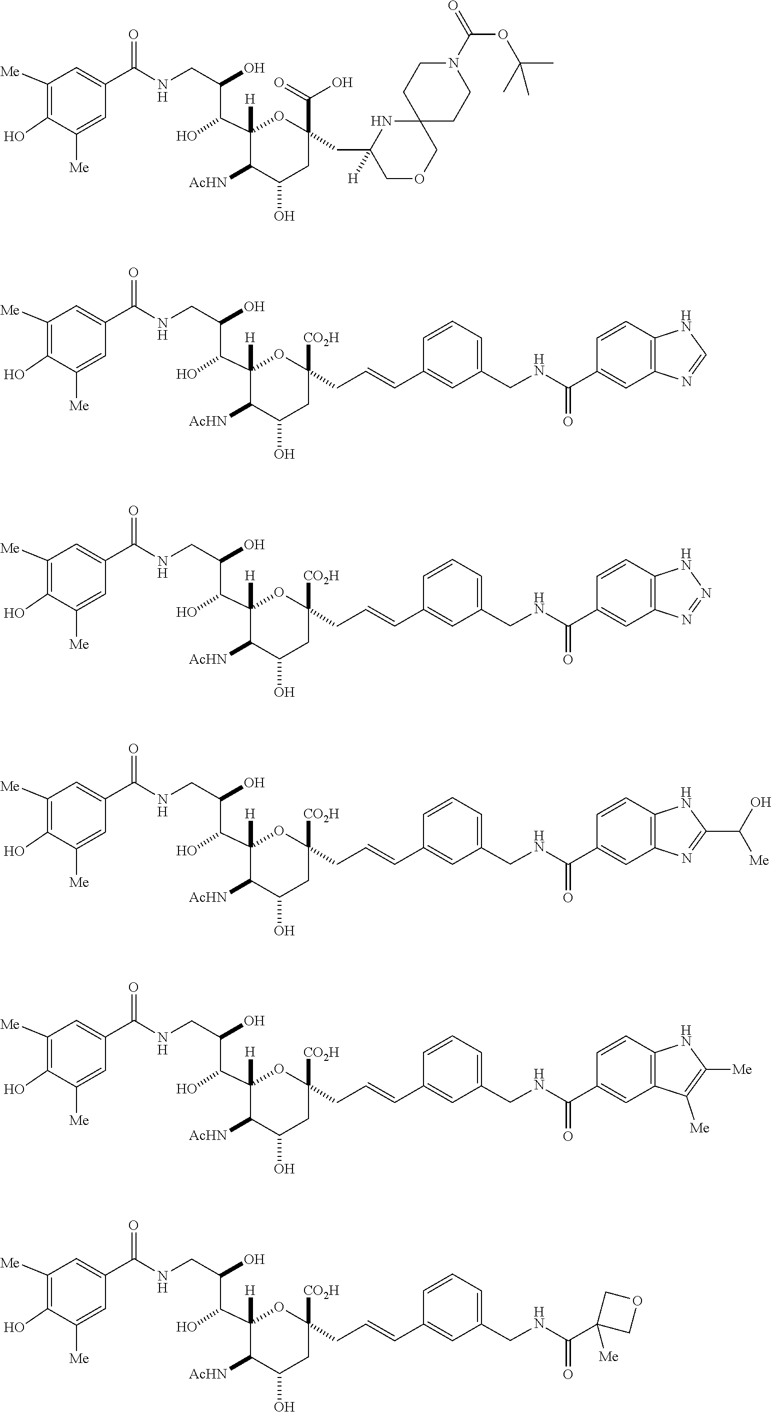

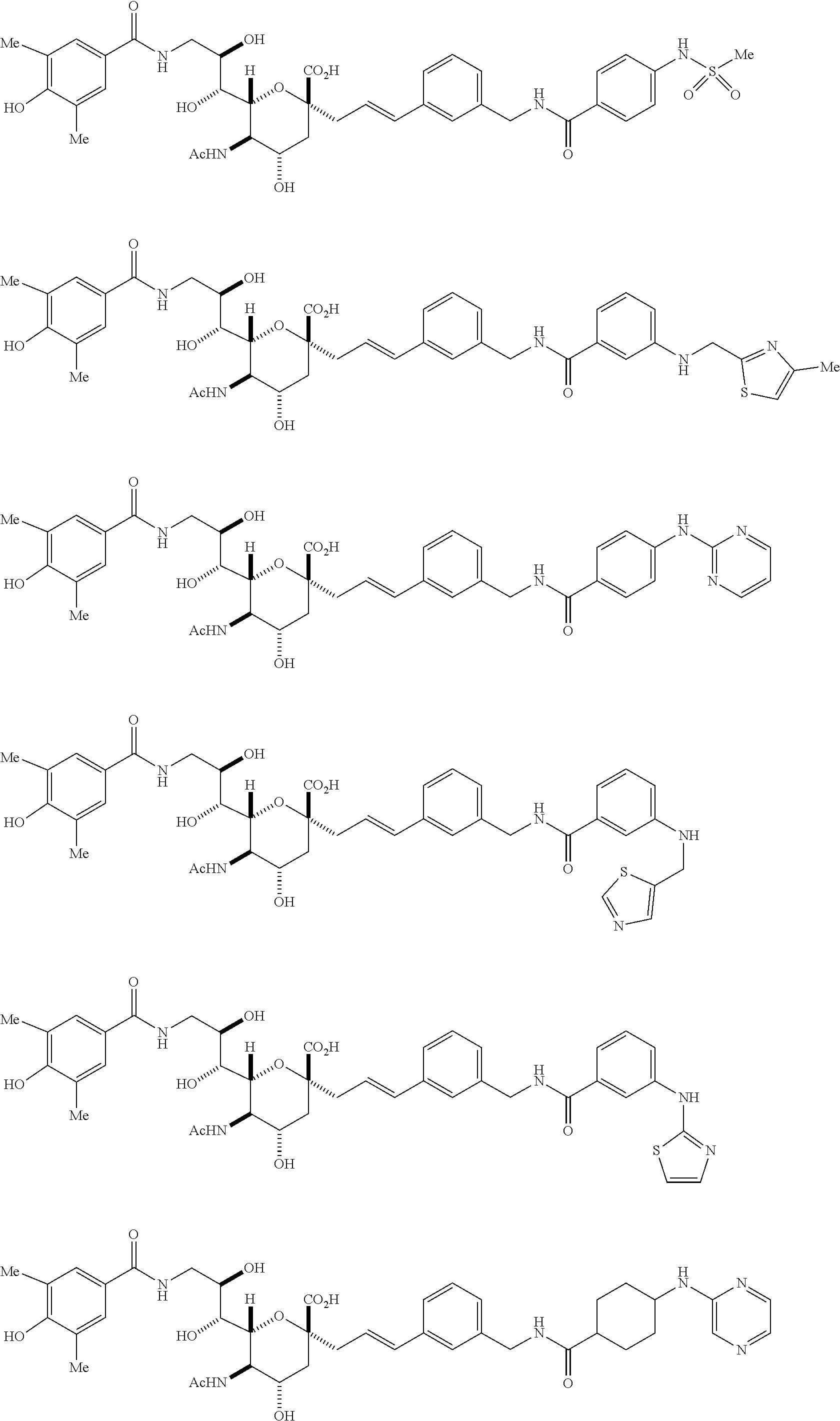

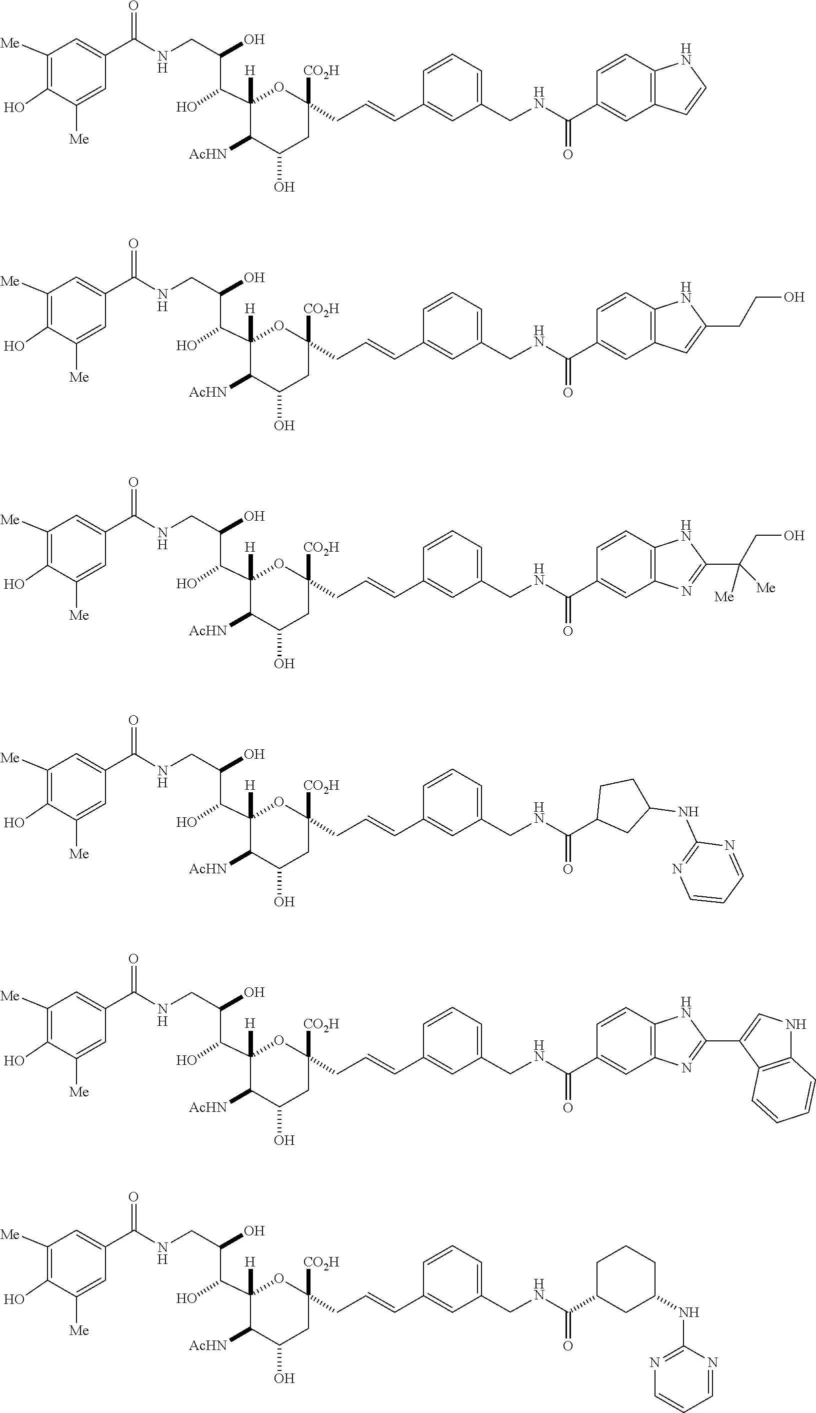

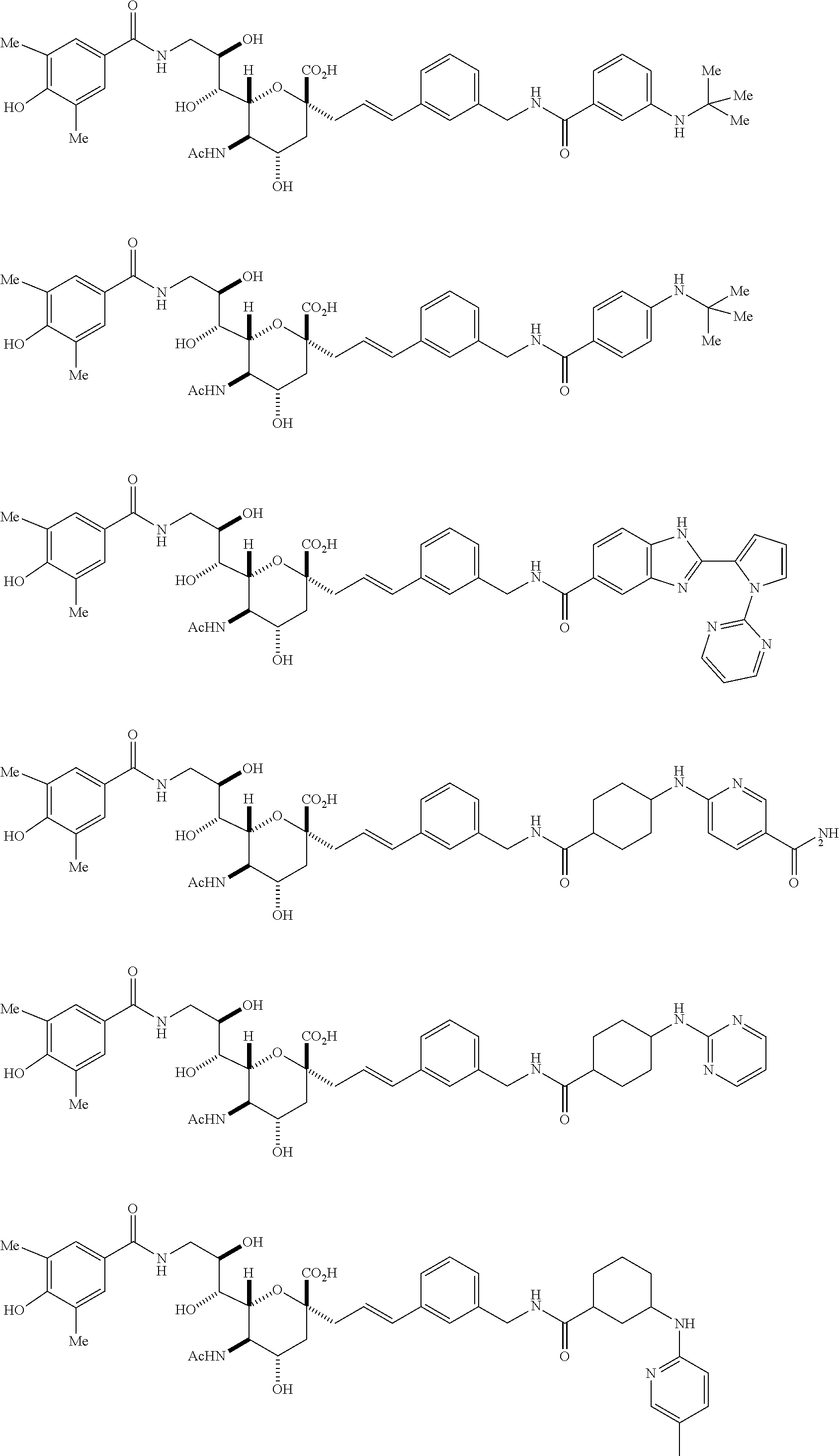

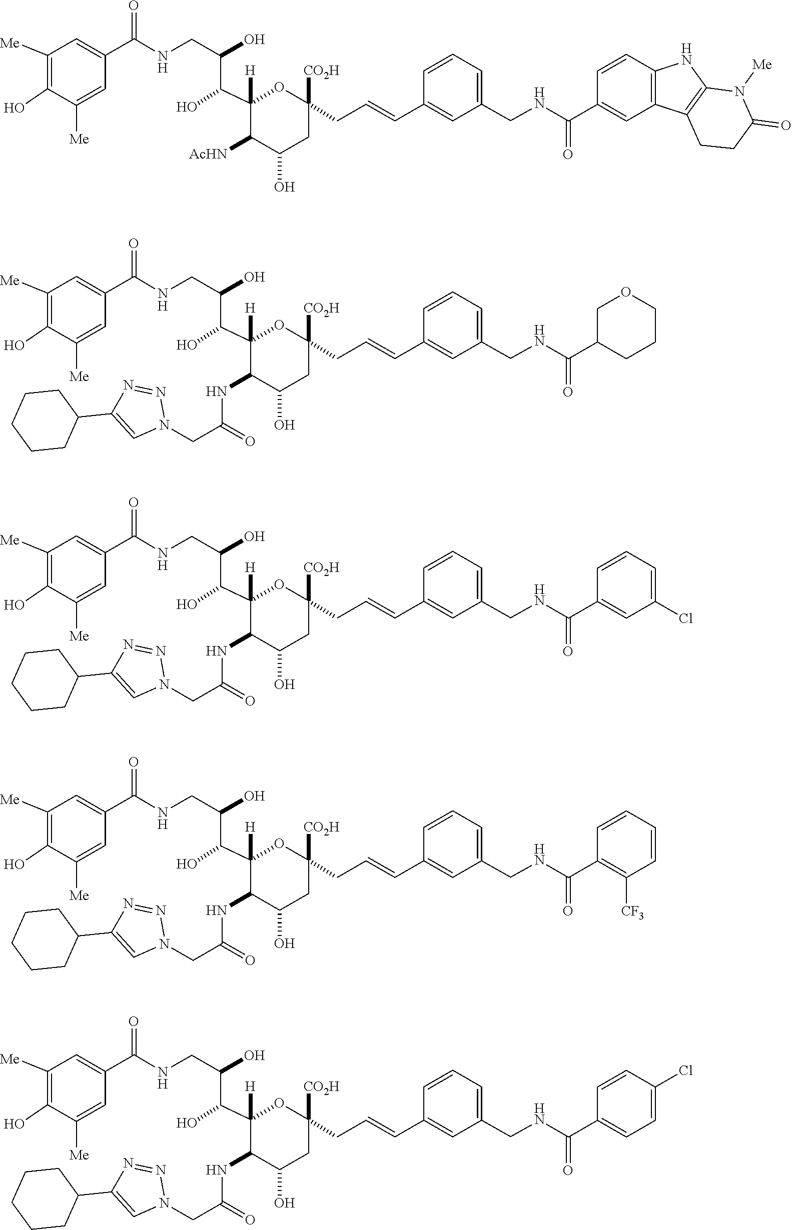

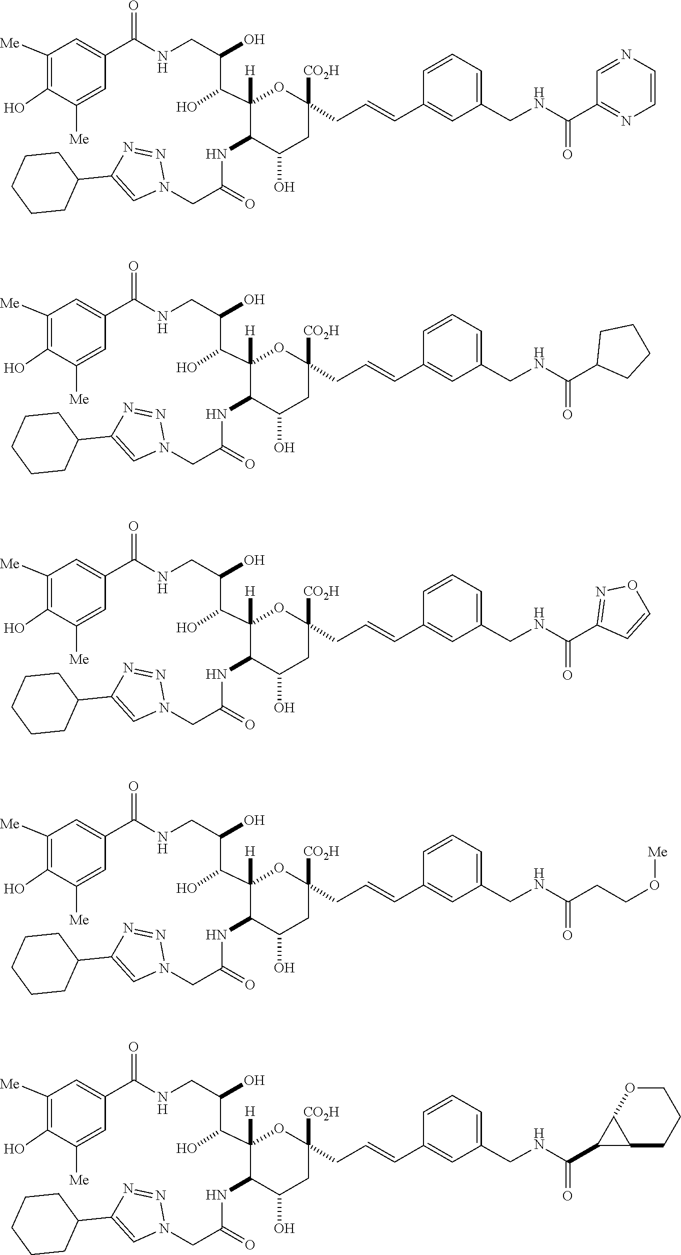

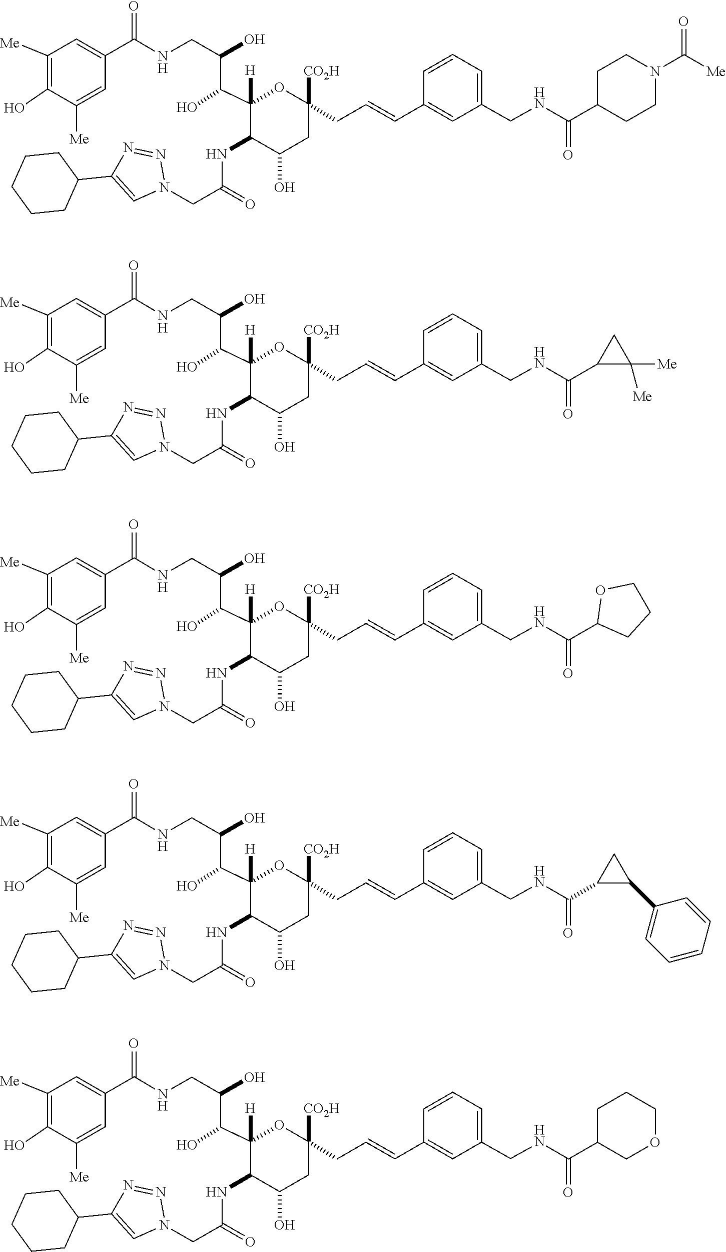

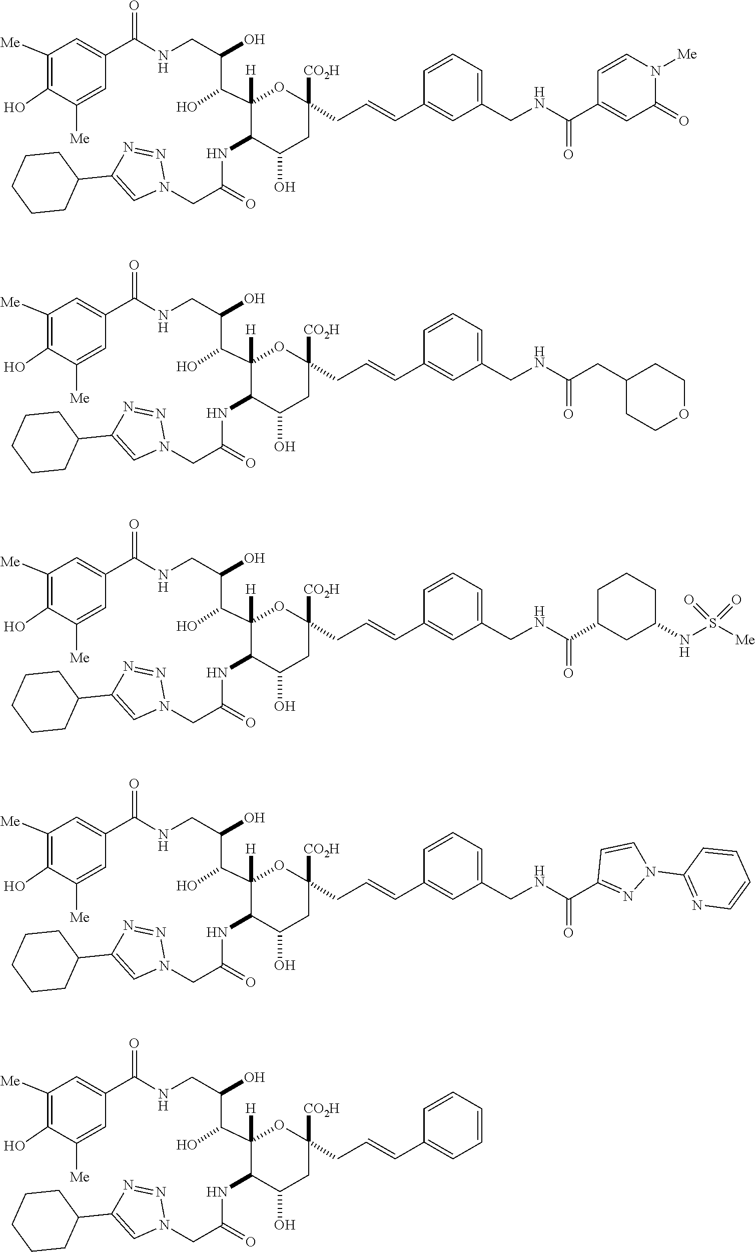

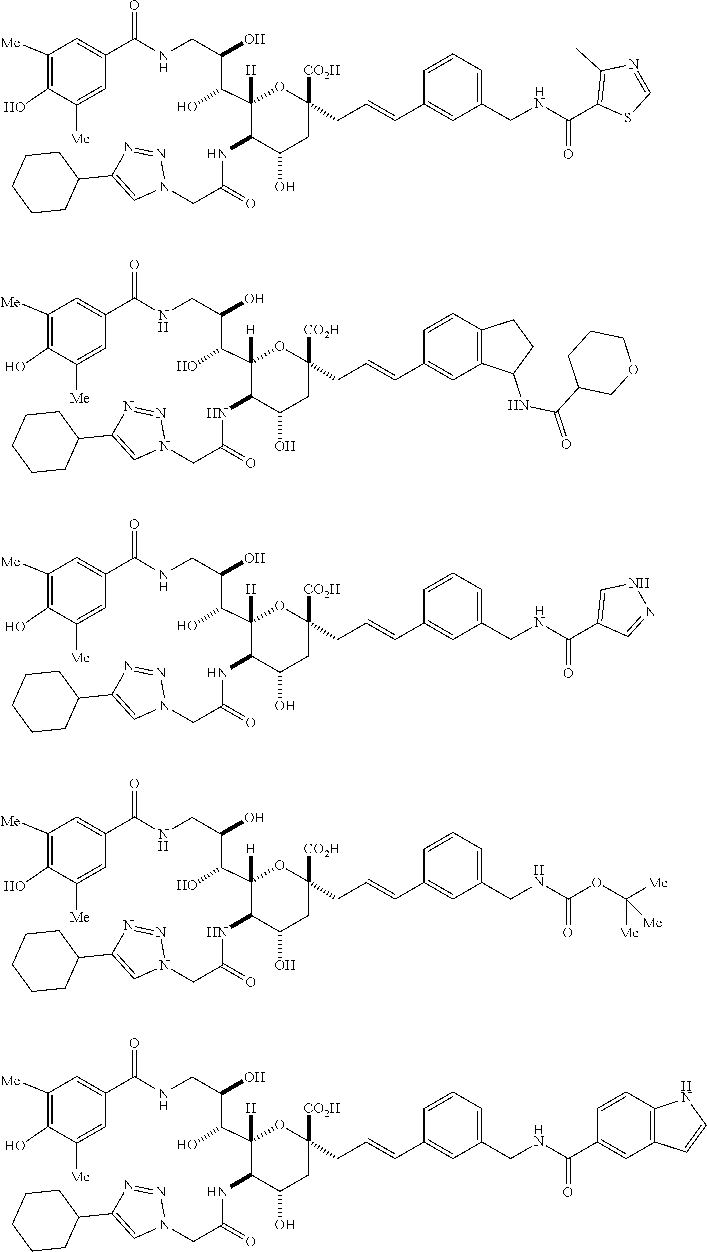

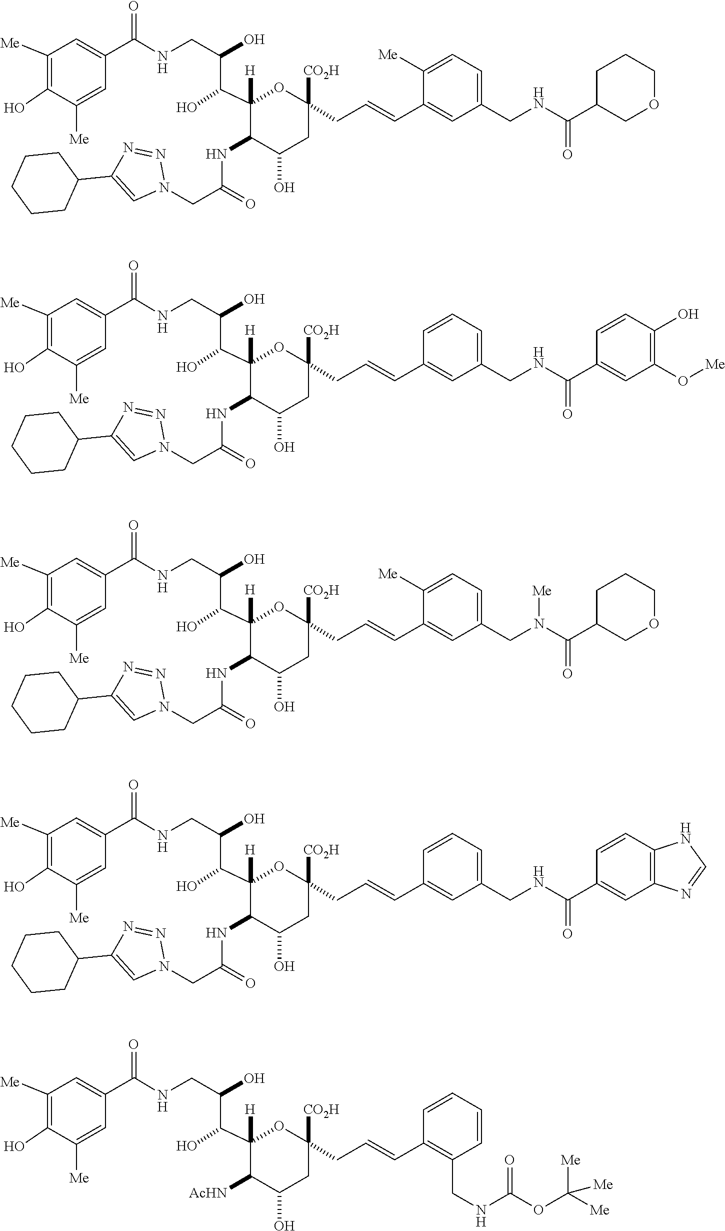

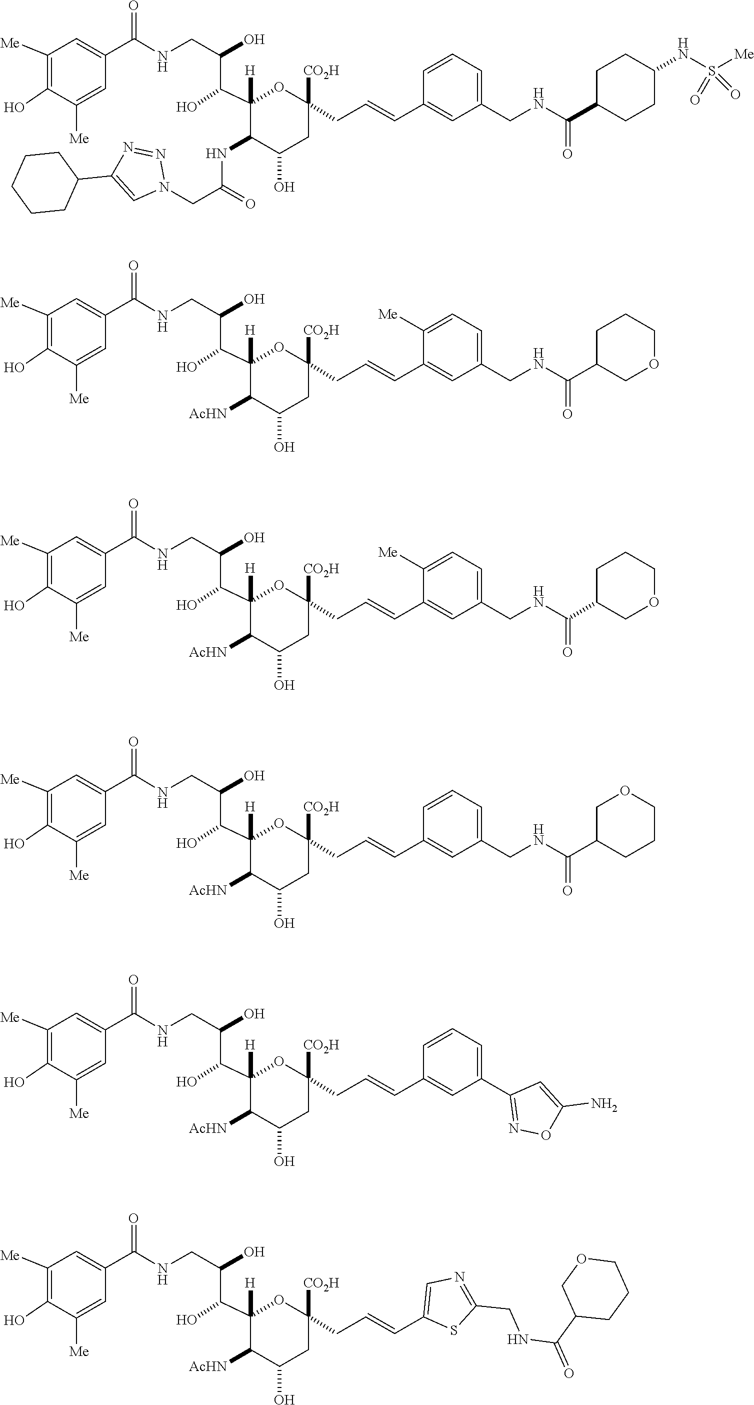

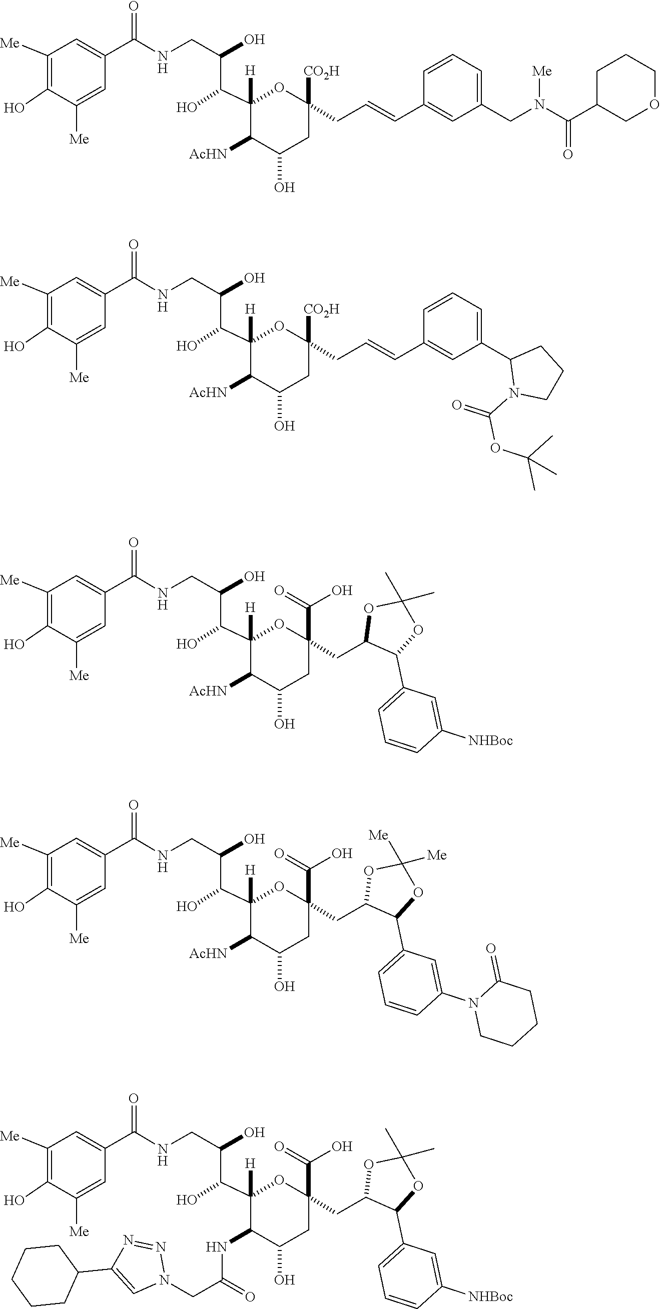

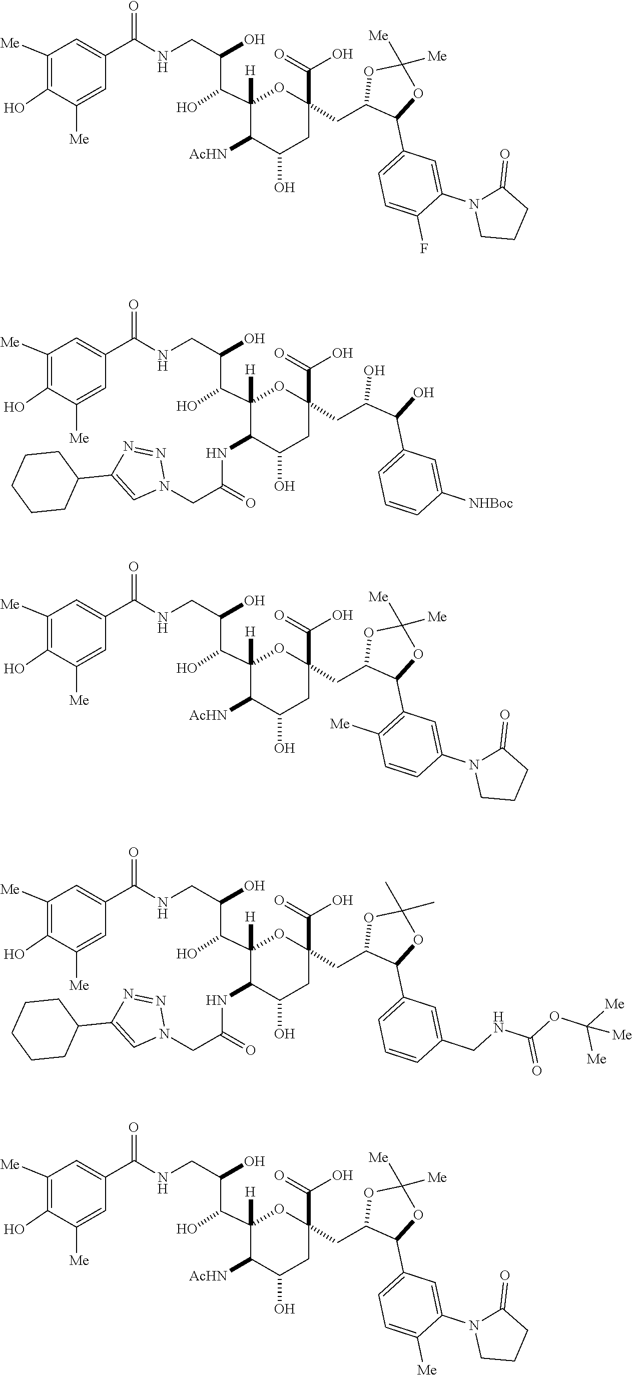

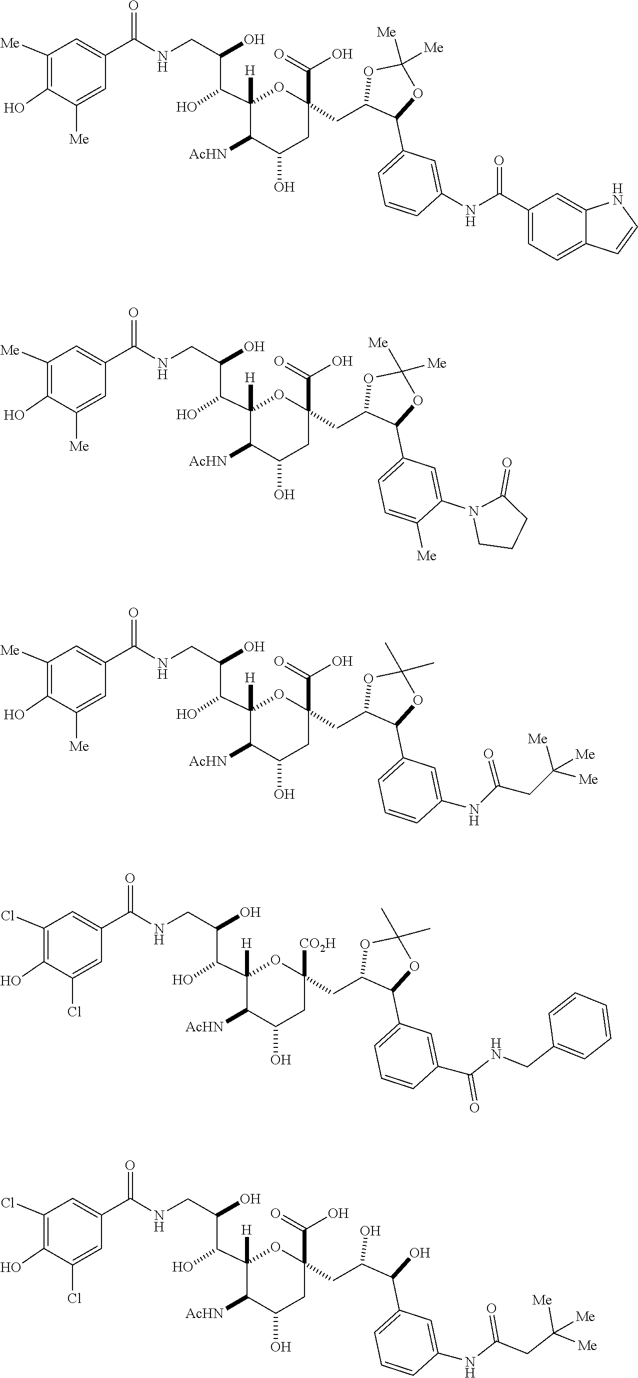

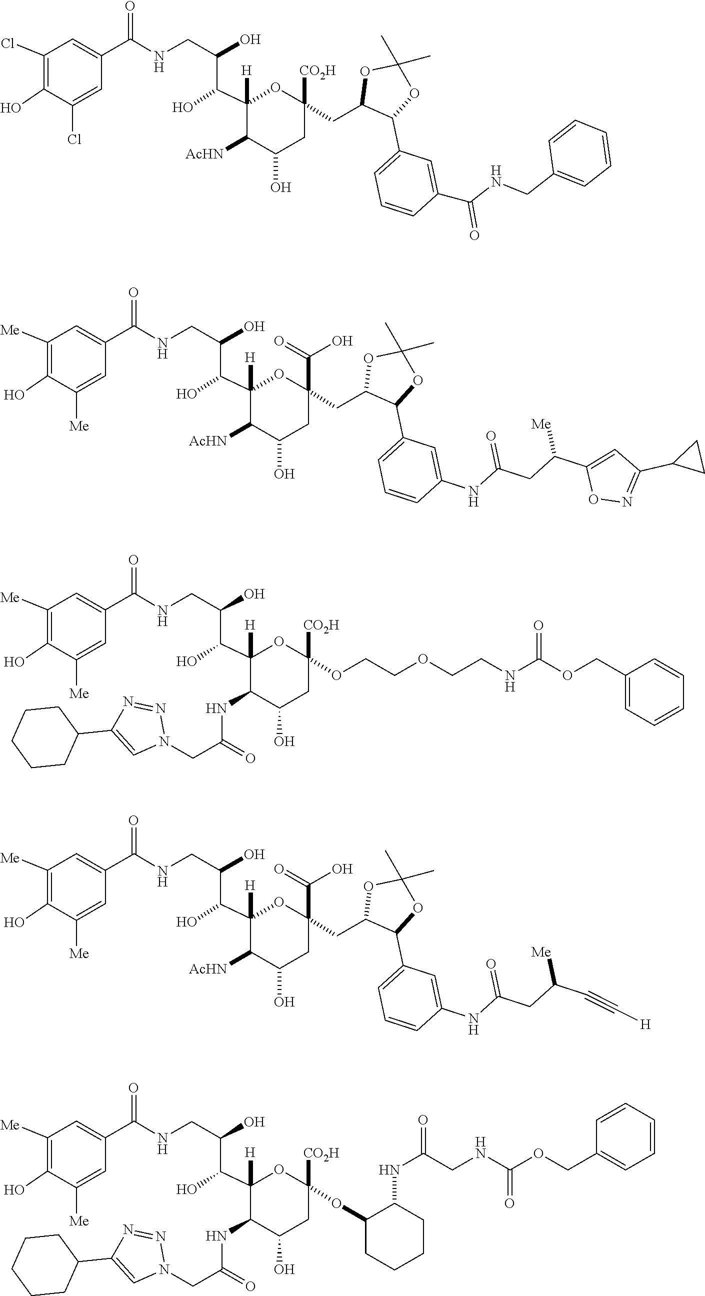

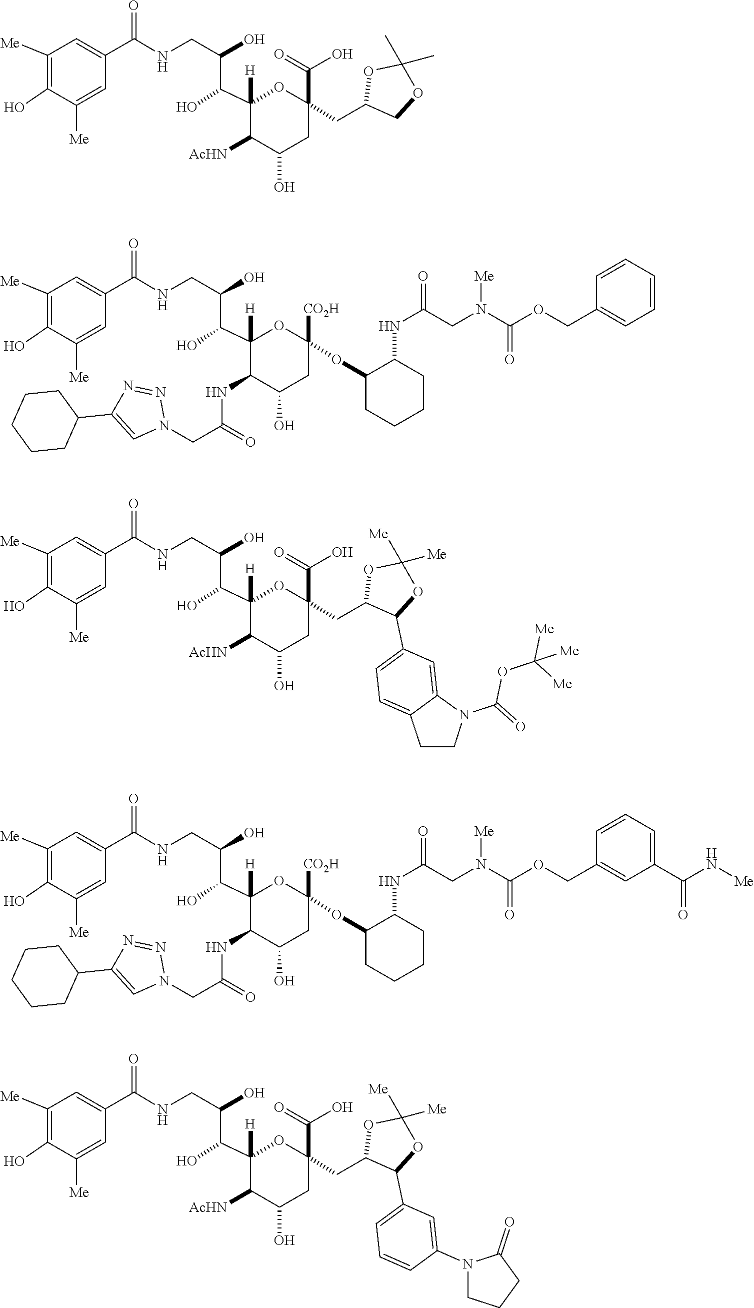

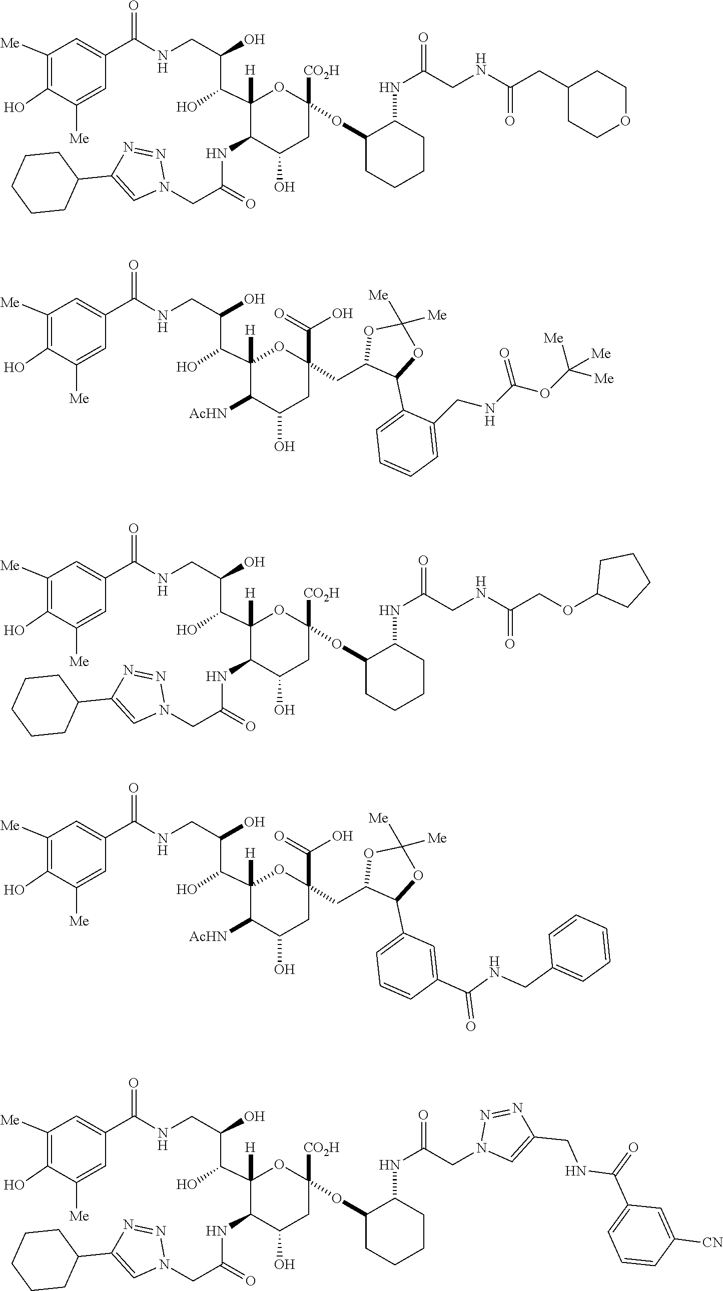

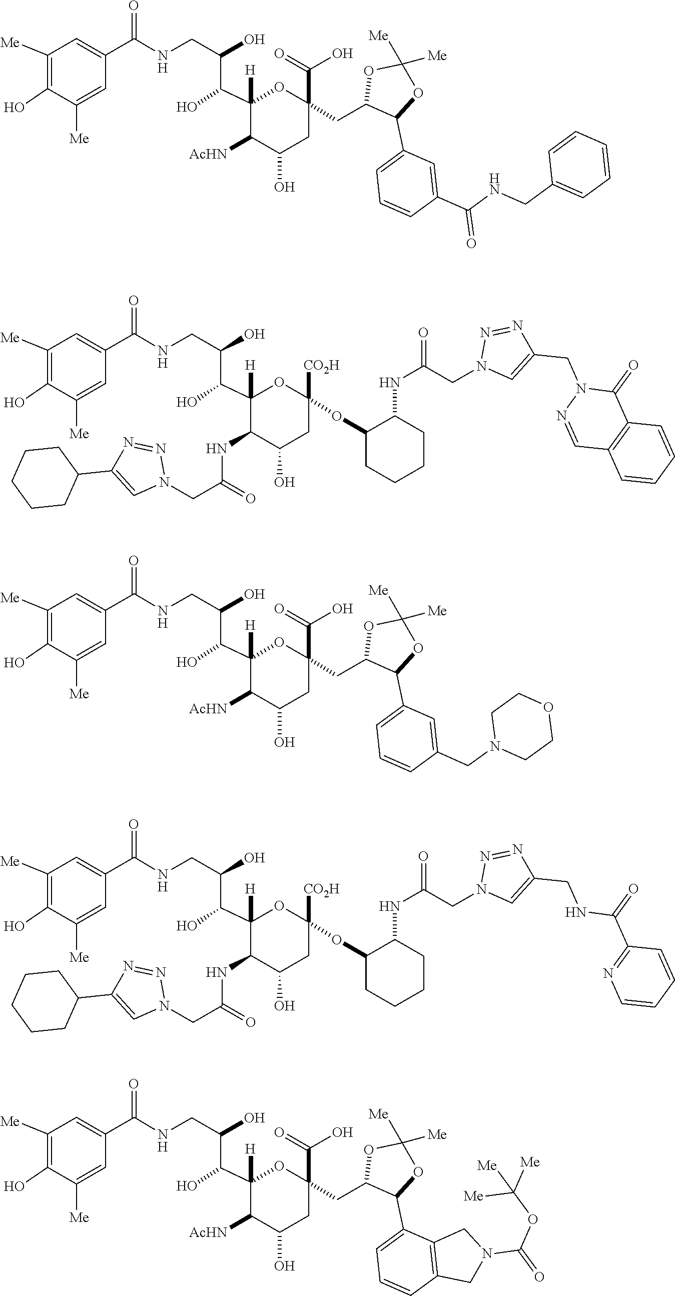

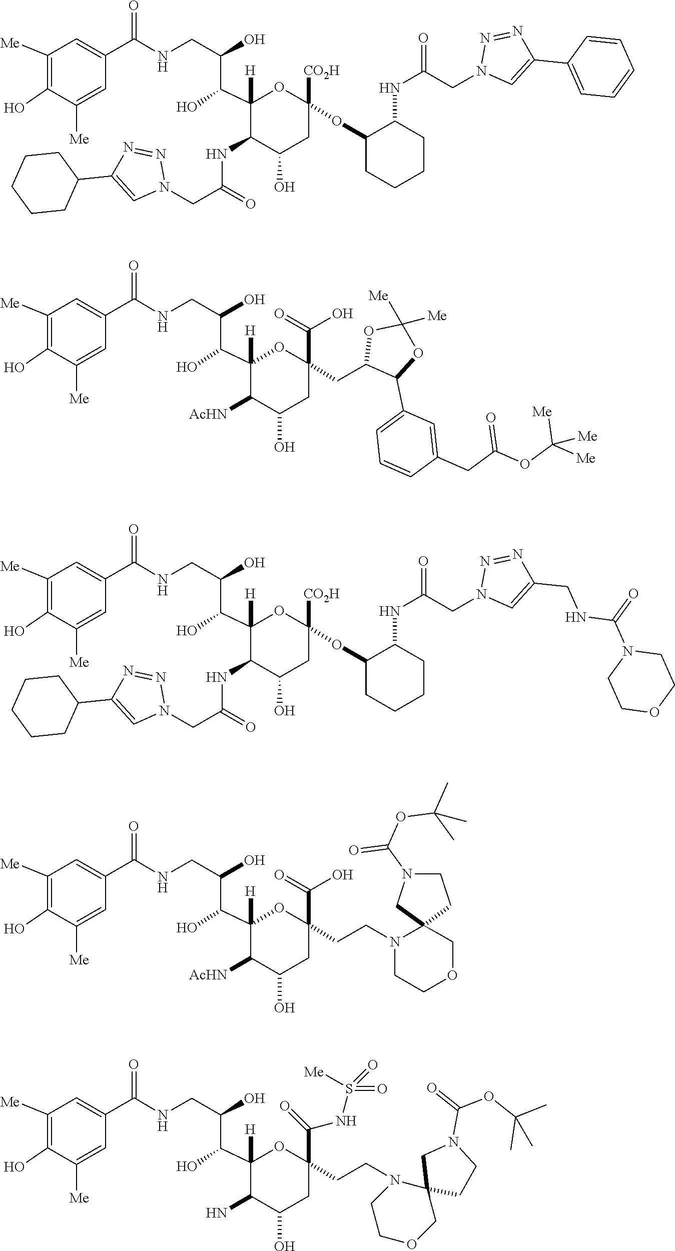

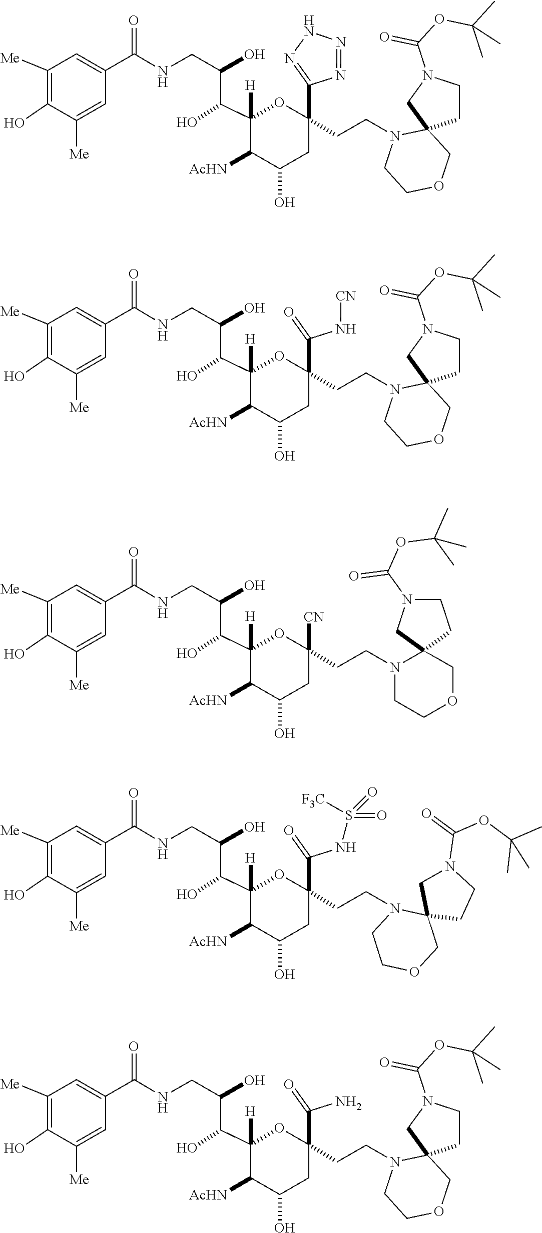

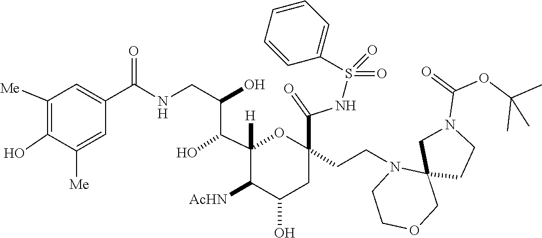

tautomers thereof, deuterated derivatives thereof, deuterated derivatives of tautomers thereof, or pharmaceutically acceptable salts of any of the foregoing.

Embodiment 45. A compound of Formula (o):

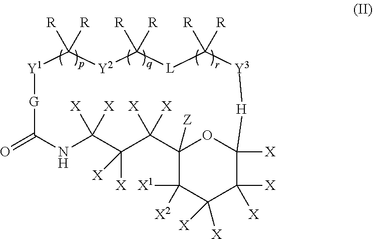

a tautomer thereof, a deuterated derivative of a compound of Formula (11), a deuterated derivative of a tautomer of a compound of Formula (11), or a pharmaceutically acceptable salt of any of the foregoing, wherein:
(i) G is chosen from cycloalkyl groups, heterocyclic groups, aryl groups, and heteroaryl groups;
(ii) Y¹ is absent or —O—;
(iii) Y² is absent or chosen from —O—, —NHC(O)—, and aryl groups;
(iv) Y³ is absent or chosen from —O—, and aryl groups;
(v) H is chosen from C₁₋₁₀ linear alkylene groups, C₃₋₁₀ branched alkylene groups, C₃₋₁₀cyclic alkylene groups, —C(O)—C₁₋₁₀ linear alkylene groups, —C(O)—C₃₋₁₀ branched alkylene groups, —C(O)—C₃₋₁₀ cyclic alkylene groups, C₁₋₁₀ linear alkylene-C(O)— groups, C₃₋₁₀ branched alkylene-C(O)— groups, C₃₋₁₀ cyclic alkylene-C(O)— groups, C₁₋₁₀ linear alkenylene groups, C₃₋₁₀ branched alkenylene groups, and C₃₋₁₀cyclic alkenylene groups;
(vi) p, q, and r are independently chosen from 0, 1, 2, 3, 4, 5, and 6;
(vii) each R is independently chosen from hydrogen, hydroxy groups, amino groups, linear alkyl groups, branched alkyl groups, cyclic alkyl groups, linear alkoxy groups, branched alkoxy groups, and cyclic alkoxy groups;
(viii) L absent or is chosen from:

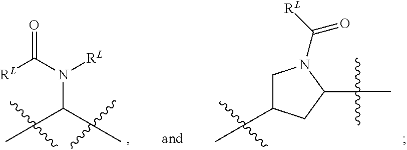

wherein R^{L} is chosen hydrogen, linear alkyl groups, branched alkyl groups, cyclic alkyl groups, heterocyclic groups, aryl groups, and heteroaryl groups;
(ix) each X is independently chosen from hydrogen, hydroxy groups, amino groups, linear alkyl groups, branched alkyl groups, and cyclic alkyl groups;
(x) X¹ and X² are each independently chosen from hydrogen, hydroxy groups, amino groups, linear alkyl groups, branched alkyl groups, cyclic alkyl groups, —NHC(O)alkylgroups, —NHC(O)arylalkylgroups, and —NHC(O)heteroarylalkylgroups;
(xi) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z},
   —C(O)NHSO₂R^{z}; wherein R^{z} is chosen from hydrogen, linear alkyl groups, branched alkyl groups, cyclic alkyl groups, carbocyclic groups, amino groups, heterocyclic groups, aryl groups, and heteroaryl groups;
wherein the linear alkyl groups, branched alkyl groups, and cyclic alkyl groups, linear alkenyl groups, branched alkenyl groups, and cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 46. A compound of Formula (IIa):

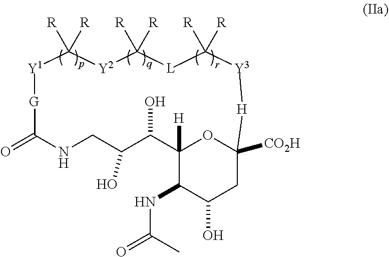

a tautomer thereof, a deuterated derivative of a compound of Formula (IIa), a deuterated derivative of a tautomer of a compound of Formula (IIa), or a pharmaceutically acceptable salt of any of the foregoing, wherein:
(i) G is chosen from cycloalkyl groups, heterocyclic groups, aryl groups, and heteroaryl groups;
(ii) Y¹ is absent or —O—;
(iii) Y² is absent or chosen from —O—, —NHC(O)—, and aryl groups;
(iv) Y³ is absent or chosen from —O—, and aryl groups;
(v) H is chosen from C₁₋₁₀ linear alkylene groups, C₃₋₁₀ branched alkylene groups, C₃₋₁₀ cyclic alkylene groups, —C(O)—C₁₋₁₀ linear alkylene groups, —C(O)—C₃₋₁₀ branched alkylene groups, —C(O)—C₃₋₁₀ cyclic alkylene groups, C₁₋₁₀ linear alkylene-C(O)— groups, C₃₋₁₀ branched alkylene-C(O)— groups, C₃₋₁₀ cyclic alkylene-C(O)— groups, C₁₋₁₀ linear alkenylene groups, C₃₋₁₀ branched alkenylene groups, and C₃₋₁₀cyclic alkenylene groups;
(vi) p, q, and r are independently chosen from 0, 1, 2, 3, 4, 5, and 6;
(vii) each R is independently chosen from hydrogen, hydroxy groups, amino groups, linear alkyl groups, branched alkyl groups, cyclic alkyl groups, linear alkoxy groups, branched alkoxy groups, and cyclic alkoxy groups;
(viii) L absent or is chosen from:

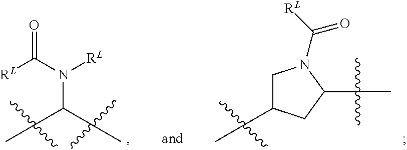

wherein R^{L} is chosen hydrogen, linear alkyl groups, branched alkyl groups, cyclic alkyl groups, heterocyclic groups, aryl groups, and heteroaryl groups; wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O— heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

Embodiment 47. The compound of any of claims 45-46, wherein G is chosen from aryl groups.

Embodiment 48. The compound of claim 47, wherein G is

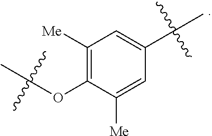 [SMILES: *Oc1c(C)cc(*)cc1C]

Embodiment 49. The compound of claim 47, wherein G is

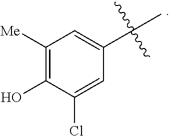 [SMILES: *c1cc(C)c(O)c(Cl)c1]

Embodiment 50. The compound of claim 45 or 46, chosen from:

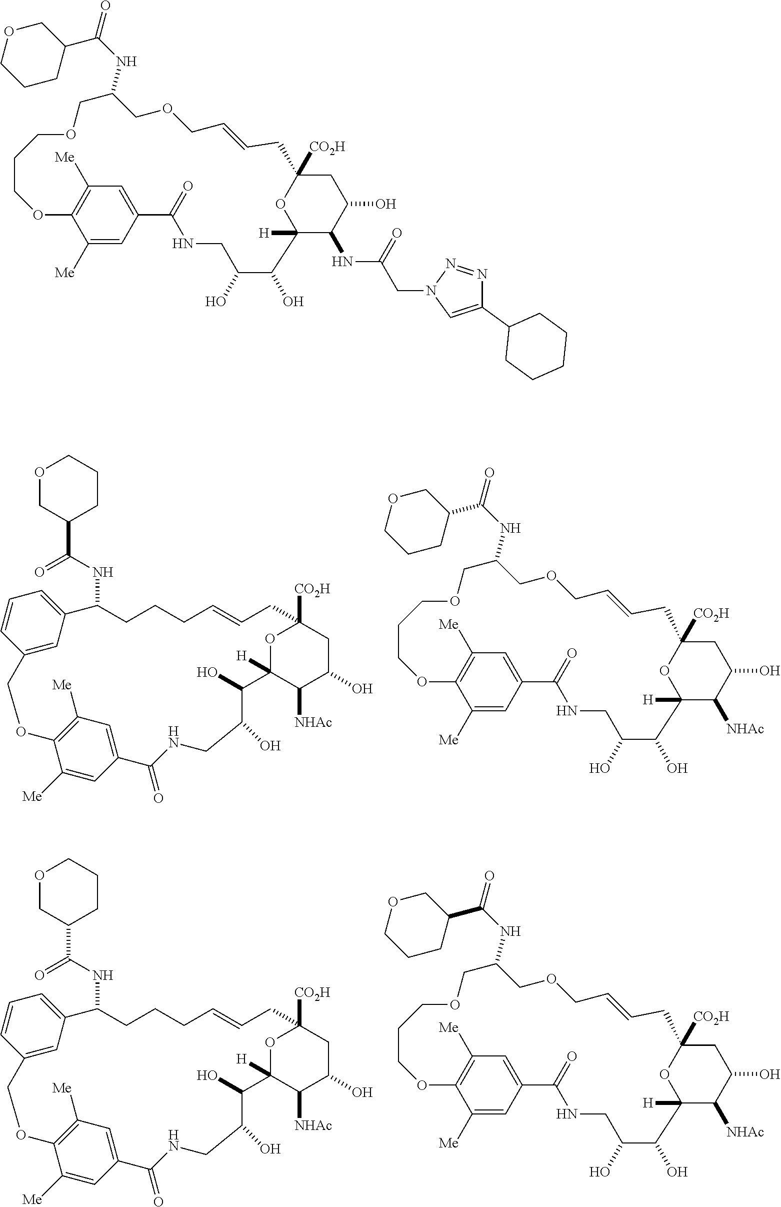

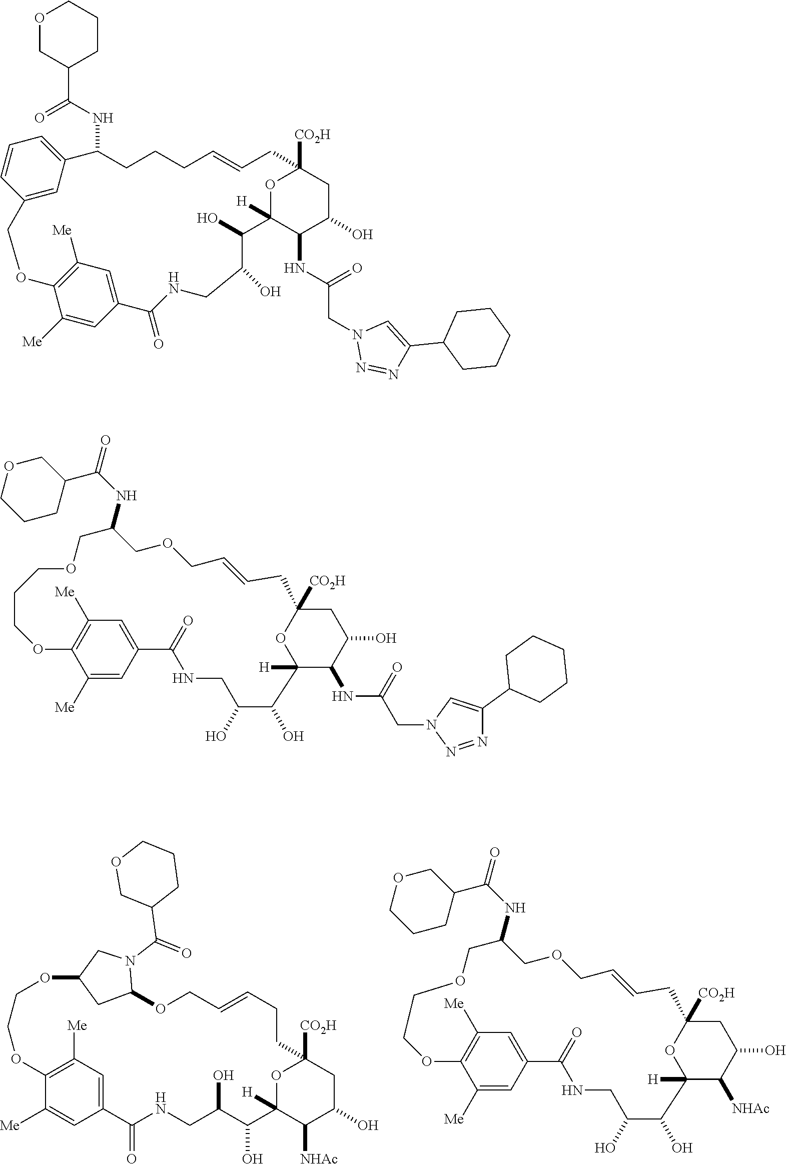

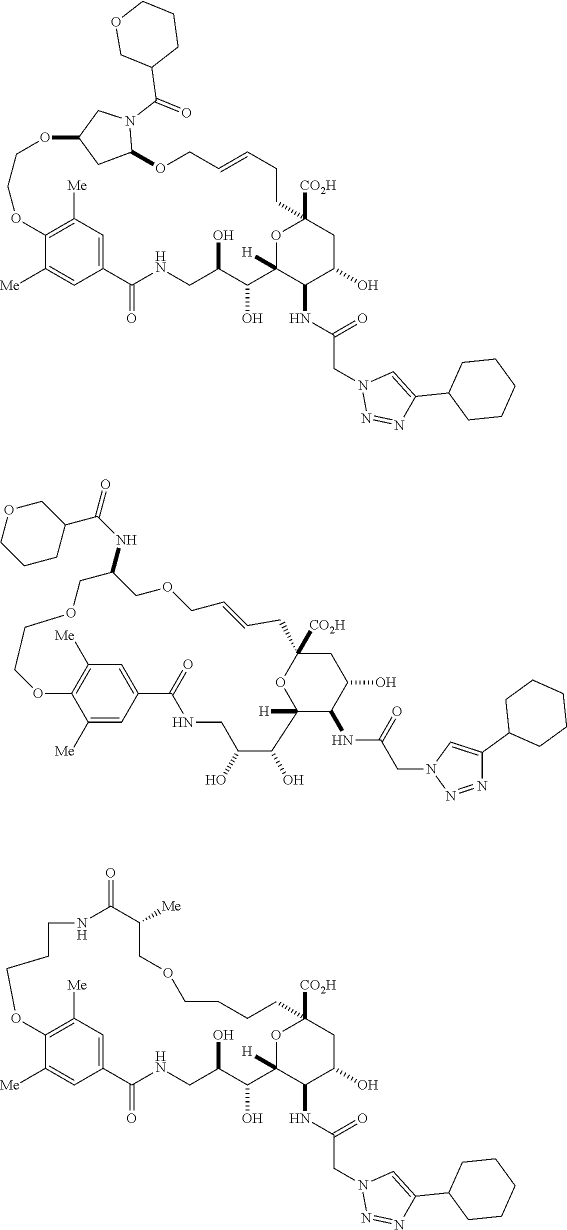

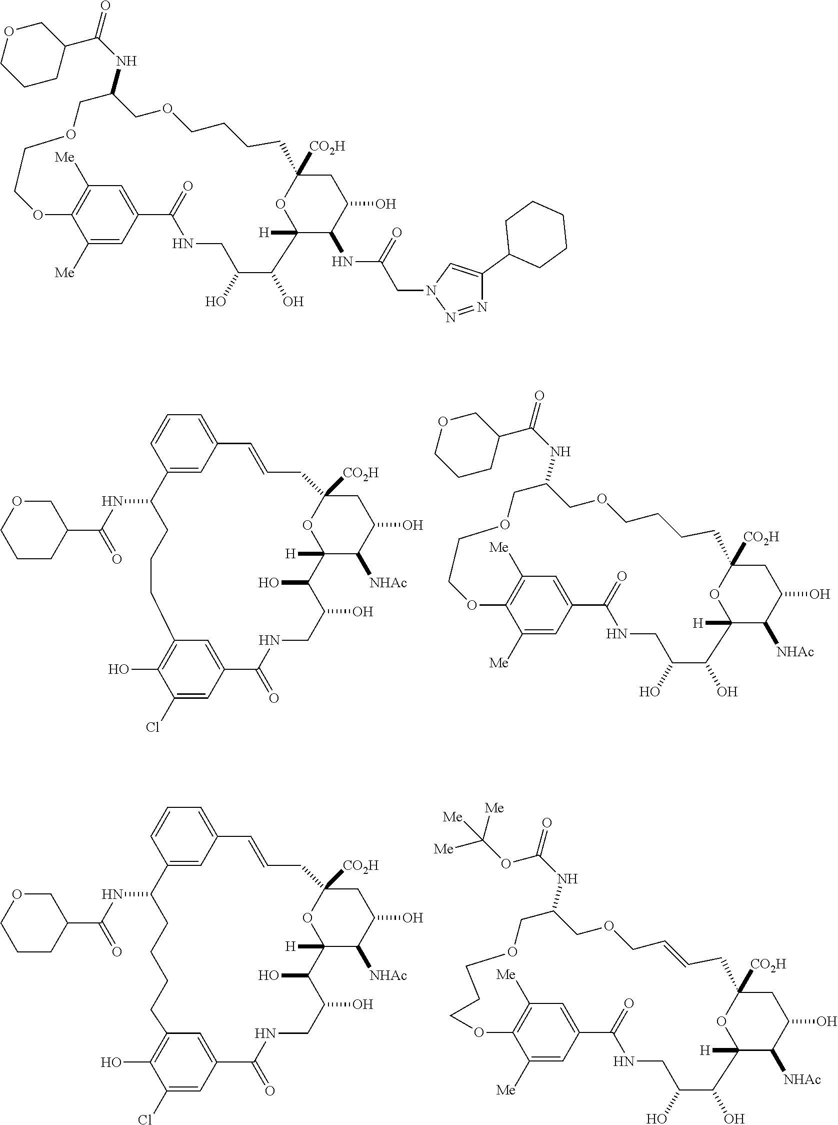

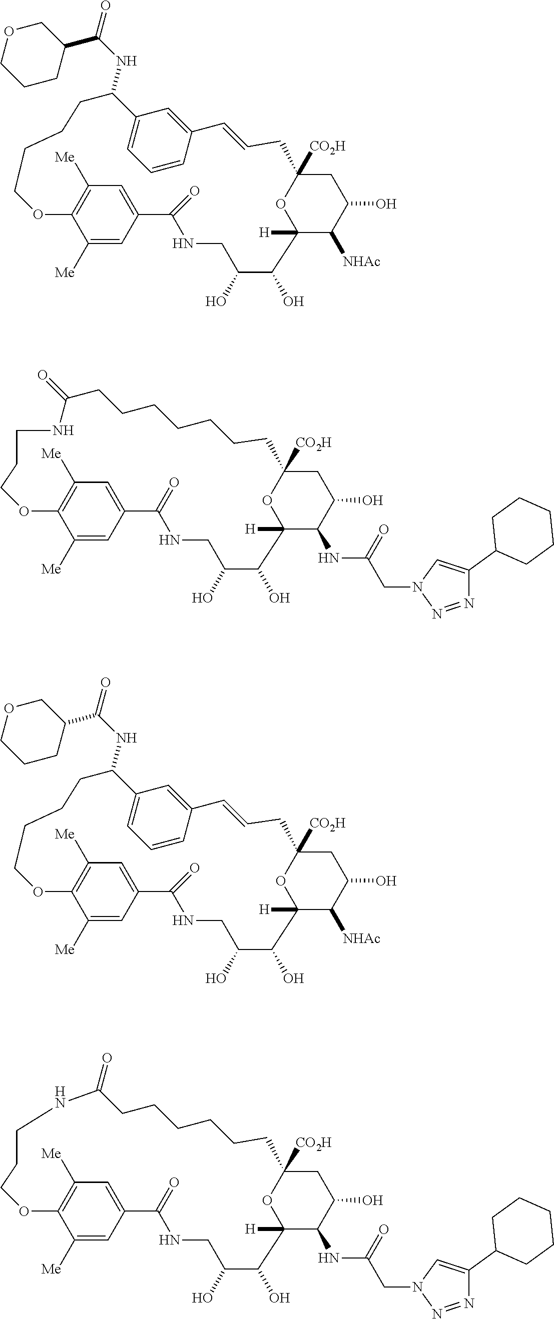

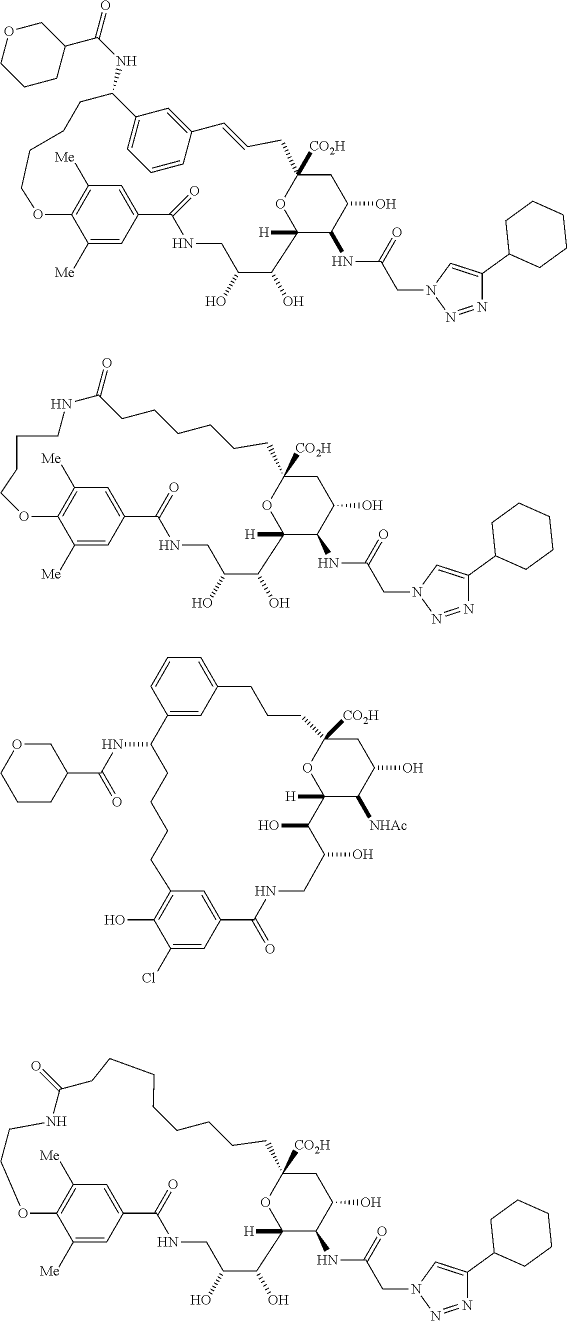

tautomers thereof, deuterated derivatives thereof, deuterated derivatives of tautomers thereof, and pharmaceutically acceptable salts of any of the foregoing.

Embodiment 51. A compound of Formula (III), (TV), or (V):

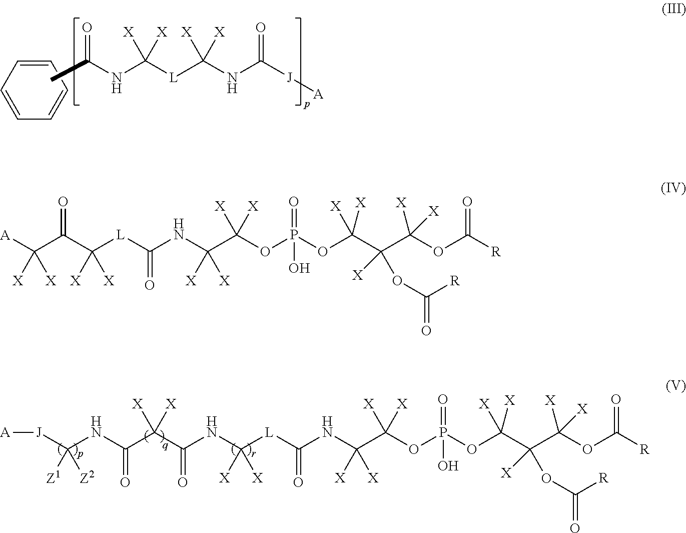

a tautomer thereof, a deuterated derivative of a compound of Formula (III), (IV), or (V), a deuterated derivative of a tautomer of a compound of Formula (III), (IV), or (V), or a pharmaceutically acceptable salt of any of the foregoing, wherein:
(i) A is a compound of any of claims 1-50;
(ii) J is chosen from cycloalkyl groups, heterocyclic groups, aryl groups, and heteroaryl groups;
(iii) Z¹, Z², and each X are each independently chosen from hydrogen, hydroxy groups, amino groups, linear alkyl groups, branched alkyl groups, and cyclic alkyl groups;
(iv) L is

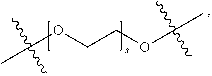

wherein s is 1-50.
(v) p, q, and r are independently chosen from 1, 2, 3, 4, 5, and 6.

Embodiment 52. The compound of Formula (III) of claim 51, wherein:
(i) A is a compound of any of claims 1-50;
(ii) J is chosen from cycloalkyl groups, heterocyclic groups, aryl groups, and heteroaryl groups;
(iii) each X is independently chosen from hydrogen, hydroxy groups, amino groups, linear alkyl groups, branched alkyl groups, and cyclic alkyl groups;
(iv) L is

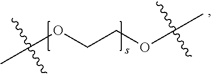

wherein s is 1-10.
(v) p is chosen from 1, 2, and 3.

Embodiment 53. The compound of Formula (IV), wherein:
(i) A is a compound of any of claims 1-50;
(ii) J is chosen from cycloalkyl groups, heterocyclic groups, aryl groups, and heteroaryl groups;
(iii) each X is independently chosen from hydrogen, hydroxy groups, amino groups, linear alkyl groups, branched alkyl groups and cyclic alkyl groups;
(iv) L is

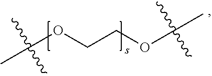

wherein s is 10-50.
(v) p, q, and r are independently chosen from 1, 2, 3, 4, 5, and 6.

Embodiment 54. The compound of Formula (V), wherein:
(i) A is a compound of any of claims 1-50;
(ii) J is chosen from cycloalkyl groups, heterocyclic groups, aryl groups, and heteroaryl groups;
(iii) Z¹, Z², and each X are independently chosen from hydrogen, hydroxy groups, amino groups, linear alkyl groups, branched alkyl groups, and cyclic alkyl groups;
(iv) L is

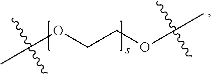

wherein s is 10-50.
(v) p, q, and r are independently chosen from 1, 2, 3, 4, 5, and 6.

Embodiment 55. A compound of any one of claims 51-54, wherein J is absent or a cyclohexyl group.

Embodiment 56. The compound of Formula (III), wherein the compound is:

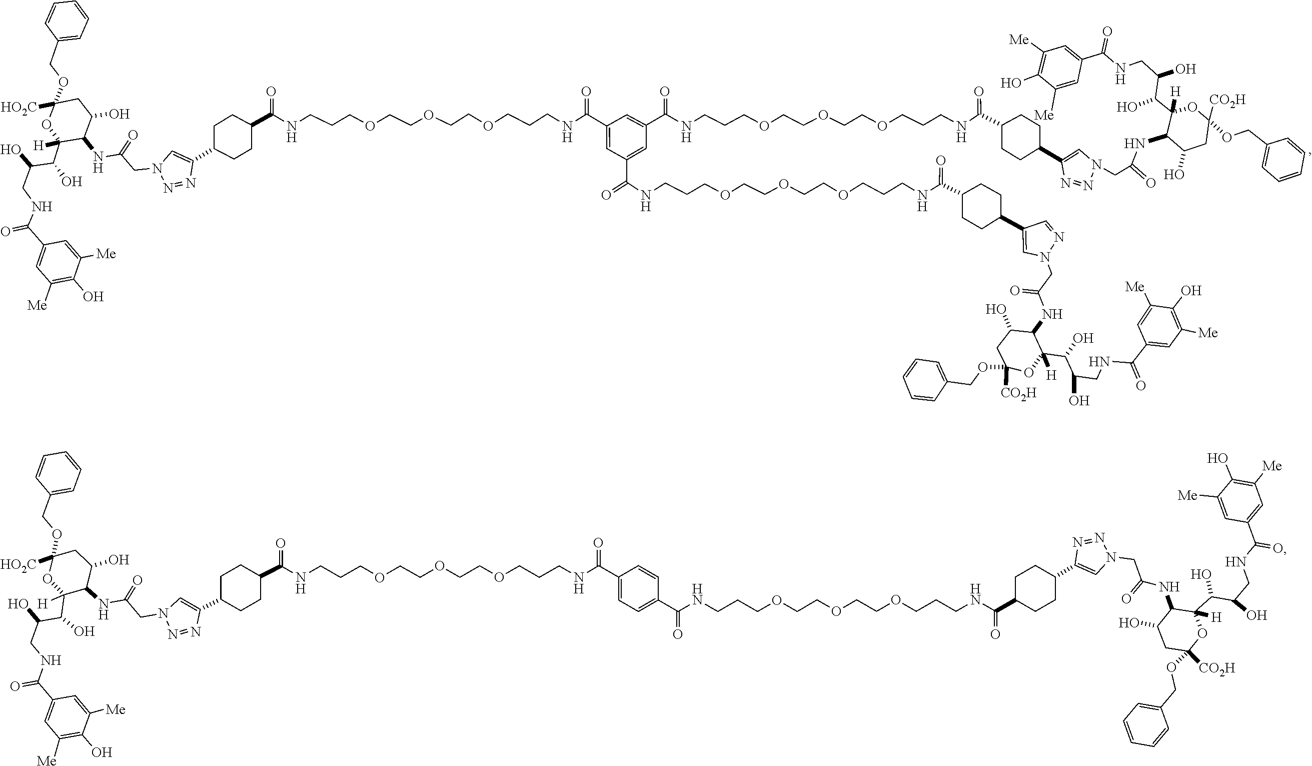

a tautomer thereof, a deuterated derivative thereof, a deuterated derivative of a tautomer thereof, or a pharmaceutically acceptable salt of any of the foregoing.

Embodiment 57. A compound of Formula (IV), wherein the compound is:

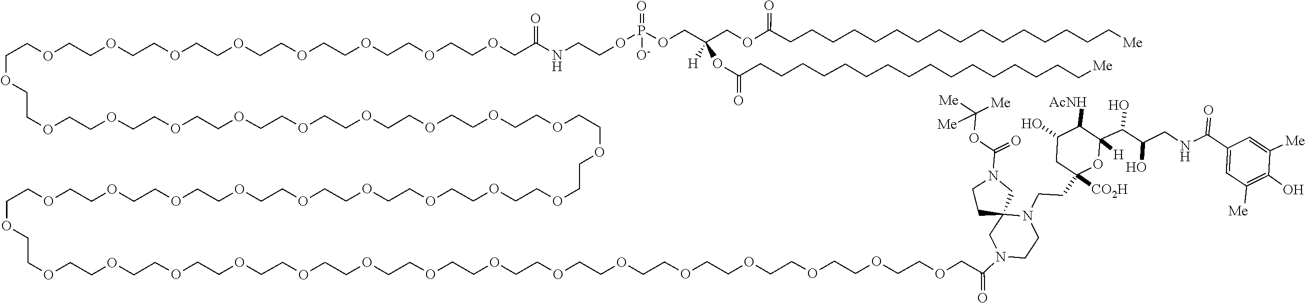

a tautomer thereof, a deuterated derivative thereof, a deuterated derivative of a tautomer thereof, or a pharmaceutically acceptable salt of any of the foregoing.

Embodiment 58. A compound of Formula (V), wherein the compound is:

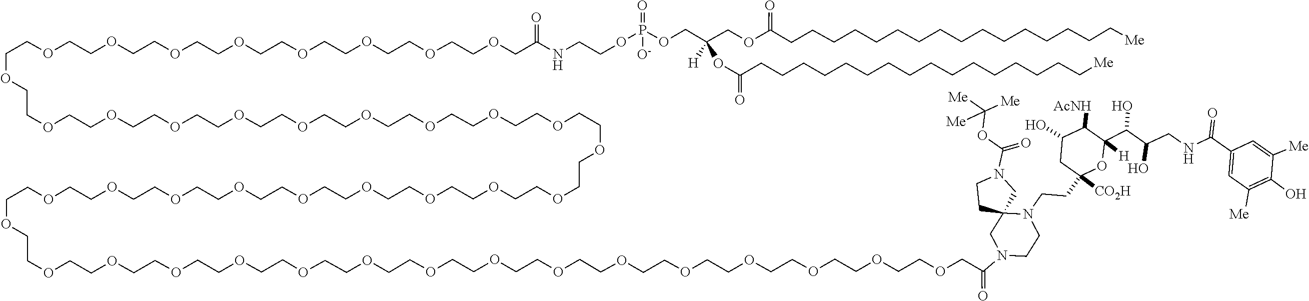

a tautomer thereof, a deuterated derivative thereof, a deuterated derivative of a tautomer thereof, or a pharmaceutically acceptable salt of any of the foregoing.

### List of Abbreviations

The following abbreviations are used herein:
AcOH: acetic acid
AF264: AlexaFluor264
anhyd: anhydrous
aq.: aqueous
Bn: benzyl
Boc: tert-butoxycabonyl
CSA: Camphor sulfonic acid
CV: column volumes
d: day(s)
DAMP: Danger-Associated Molecular Pattern
DBU: 1,8-Diazobicyclo[5.4.0]undec-7-ene
DCE: 1,2-dichloroethane
DCM: dichloromethane
DIPEA: N,N-diisopropylethylamine
DMA: N,N-Dimethylacetamide
DMAP: 4-Dimethylaminopyridine
DMF: N,N-dimethylformamide
DMSO: Dimethyl sulfoxide
dsDNA: double-stranded DNA
DSPC: distearoyl phosphatidylcholine
DSPE: distearoyl phosphatidylethanolamine
EDC: 1-(3-dimethylaminopropyl)-3-ethylcarbodiimide hydrochloride
ee: enantiomeric excess
EA: EtOAc: ethyl acetate
EtOH: ethanol
h: hour(s)
HATU: N,N,N′,N′-Tetramethyl-O-(7-azabenzotriazol-1-yl)uronium hexafluorophosphate
HCl: hydrochloric acid
HCQ: hydroxychloroquine
hep: n-heptane
HEPES: 4-(2-hydroxyethyl)-1-piperazineethanesulfonic acid
HOBₜ—1-hydroxybenzotriazole hydrate
HPLC: high performance liquid chromatography
IFN: interferon
IPA: isopropyl alcohol or isopropanol
K₂CO₃: potassium carbonate
LHMDS: lithium hexamethyldisilazide
MeOH: methanol
MgSO₄: magnesium sulfate (anhydrous)
min: minute(s)
MTBE: methyl tert-butyl ether
Na₂CO₃: sodium carbonate
Na₂SO₄: sodium sulfate (anhydrous)
NaBH₄: sodium borohydride
NaCl: sodium chloride
NaH: 60% sodium hydride dispersed in oil
NaHC03: sodium bicarbonate
NaOH: sodium hydroxide
NBS: N-bromosuccinimide
NH₄Cl: ammonium chloride
NH₄Cl: ammonium chloride
NH₄OH: ammonium hydroxide
NMP: N-methylpyrrolidone
Ns: Nosyl or o-nitrobenzenesulfonyl
° C.: degrees Celsius
PAMP: Pathogen-Associated Molecular Pattern
PBMC: peripheral blood mononuclear cell
PBS: phosphate buffered saline
pDC: plasmacytoid dendritic cell
PEG: polyethyleneglycol
PhNTf₂: N-phenyltrifluoromethanesulfonimide
qPCR: quantitative polymerase chain reaction
RT: room temperature
rt: room temperature
sat.: saturated
SOC: standard-of-care
T3P: Propylphosphonic anhydride
tBuOK: potassium tert-butyloxide
TEA: triethylamine
TEMPO: 2,2,6,6-Tetramethylpiperidine 1-oxyl
Tf: trifluoromethanesulfonate
TFA: trifluoroacetic acid
THF: tetrahydrofuran
TLC: thin layer chromatography
TLDA: Tagman@Low Density Array
TLR: Toll-like receptor
TSA: p-toluenesulfonic acid
UPLC: ultra performance liquid chromatography

### EXAMPLES

### General Procedure for the Preparation of Neuraminic Acid C-Glycosides Key Intermediate F.

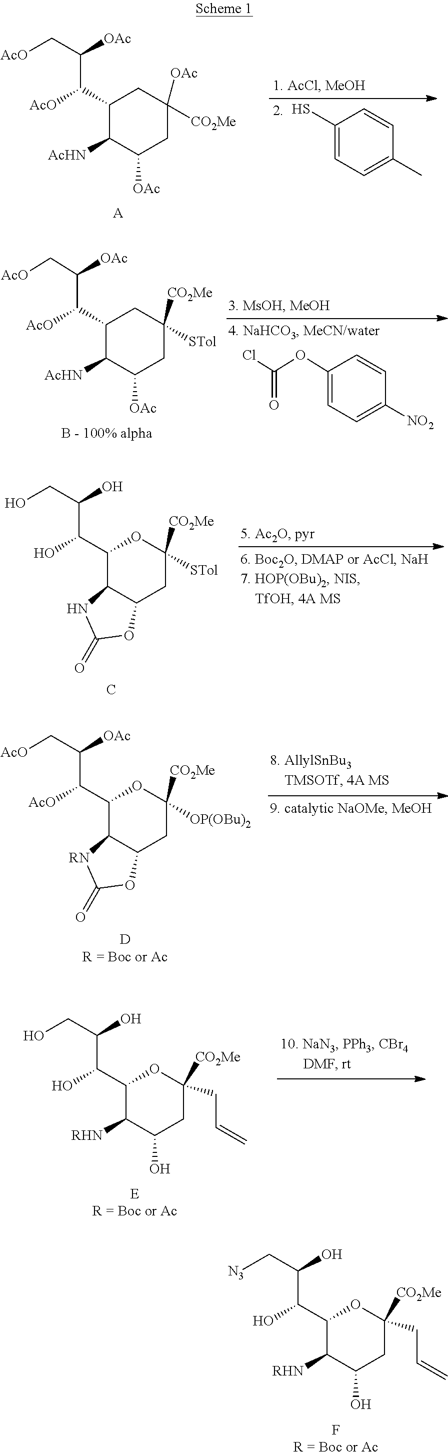

The key intermediate for the preparation of several analogs of this invention is the C-glycoside compound F, which can be obtained starting with commercially available per-acetylated neuraminic acid methyl ester, A, in 10 steps as shown in Scheme 1. Compound A is converted to the C2-thioglycoside B via a C2-chloride intermediate followed by deprotection of the acetyl groups, and formation of the C-4,5-carbamate C. Re-protection of the free hydroxyls, Boc-protection of the carbamate nitrogen allows for the formation of C2-O-protected phosphate D. Allylation of activated D under anhydrous acidic conditions followed by removal of the acetyl groups and carbamate with catalytic methoxide provides the C-glycoside E, which can be easily transformed to the C-9 azide F using sodium azide under Mitsunobu conditions.

### General Procedure for the Preparation of C2-Amino Analogs (Compounds G and H) of Neuraminic Acid C2-C-Glycosides, Option A.

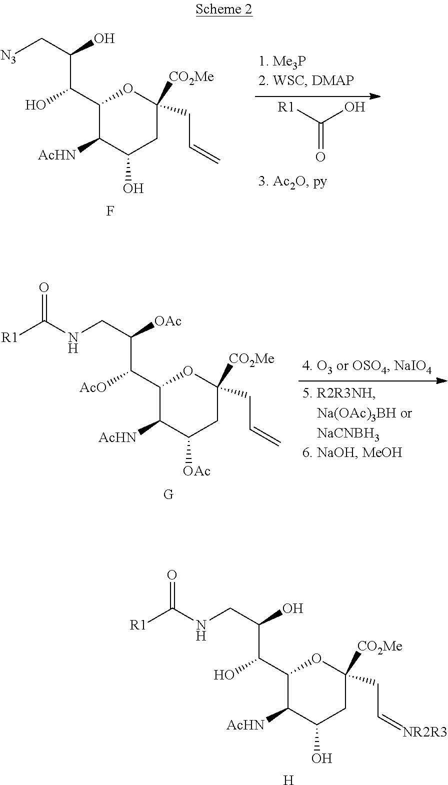

The C2-amino analogs of this invention can be prepared starting with intermediate F (R═Ac) in 6 steps as shown in Scheme 2. Reduction of the C9-azide to the primary amine followed by acylation using any carboxylic acid in the presence of water soluble carbodiimide with catalytic organic base, and finally protection of the free hydroxyl groups with acetyl groups provides compound G. Oxidation of the terminal olefin at the C2-position using ozone or osmium tetraoxide/periodate combination provides the C-2-ethanal intermediate, which allows for the formation of various amine analogs by reductive amination, and finally deprotection/ester hydrolysis to form compound H with various R, R1 and R2-substitutions as described in detail below.

General Procedure for the Preparation of C2-amino Analogs (Compounds I-H) of Neuraminic Acid C2-C-glycosides, Option B.

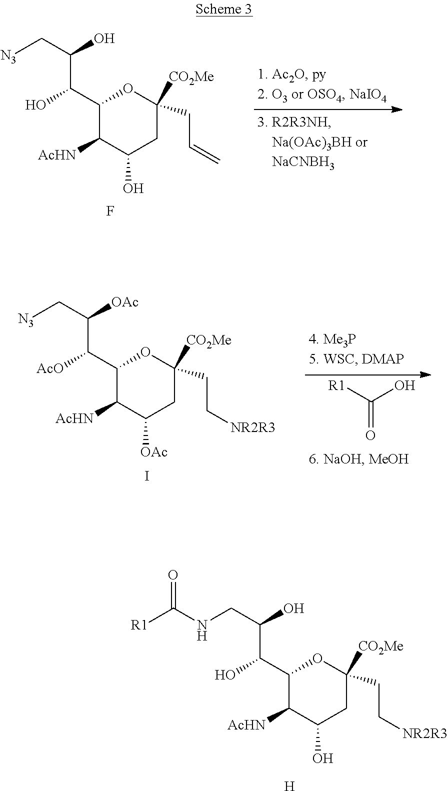

An alternative method for the preparation of C2-amino analogs (compound x-y) can be obtained by changing the order of the addition of substituents to Compound F(R═Ac) as shown in Scheme 3. Thus protection of the free hydroxyl group with acetyl, then oxidation of the terminal olefin at the C2-position using ozone or osmium tetraoxide/periodate combination provides the C-2-ethanal intermediate followed by the formation of various amine analogs by reductive amination to provided compound I. The C9-azide can then be reduced to the primary amine followed by acylation using any carboxylic acid in the presence of water soluble carbodiimide with catalytic organic base, and finally hydrolysis of the ester to provides compound H with various R, R1 and R2-substitutions as described in detail below.

General Procedure for the Preparation of C2-amino Analogs (Compounds J, and L1-L4) of Neuraminic Acid C2-C-glycosides Using the SNAP Reaction.¹

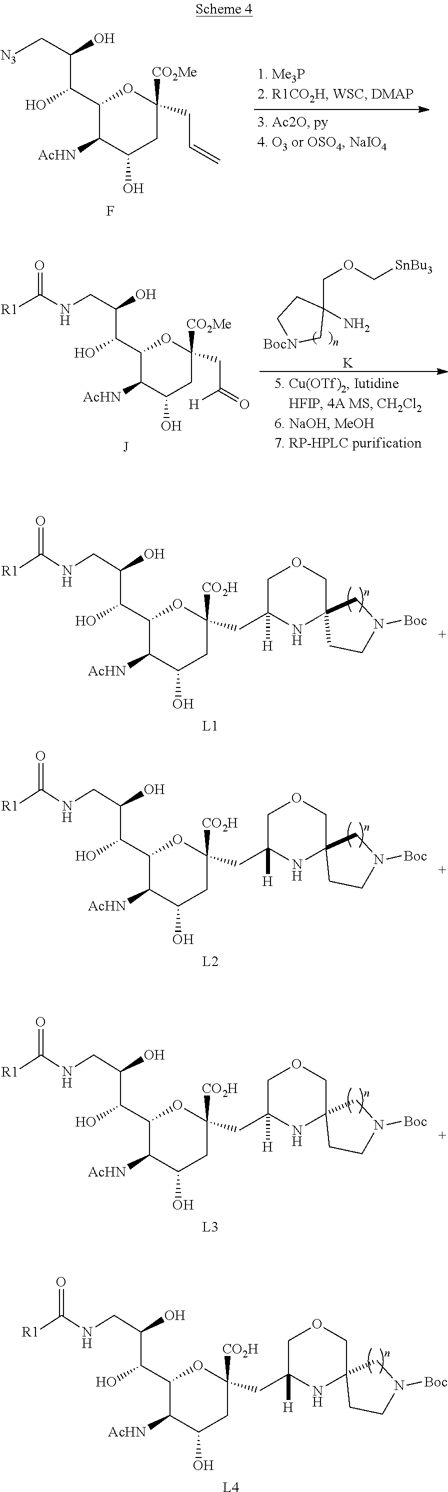

An alternative method for the generation of C2-amino analogs, or spiroamino analogs is provided in Scheme 4. Starting with intermediate F, with C5 as acetamine, one first functionalizes the C9 position by reduction of the azide followed by acylation with various carboxylic acids, and then separately generate the aldehyde J in a single step using ozone, or a two-step process of osmium tetraoxide followed by sodium periodate. The aldehyde J is then converted to the spiroamine systems L1-L4 using racemic butyl tin reagent K, where n=1 or 2) using conditions described by Luescher, et. al,¹ followed by hydrolysis. The four analogs are easily separated by using HPLC over a chiral column, and the specific stereochemistry of each isomer is determined by X-ray crystallography. Using one enantiomer of K would provide 2 of the depicted analogs. ¹M. U. Luescher, C-V. T. Vo, J. W. Bode. Org. Lett. 2014,16, 1236-1239.; K. Geoghegan, J. W. Bode. Org. Lett. 2015, 17, 1934-1937.

1. General Procedure for the Preparation of C2-C-analogs (Compounds O and P) of Neuraminic acid C2-C-glycosides using the Grubbs' or Modified Grubbs' Metathesis Reaction.^{2,3,4}

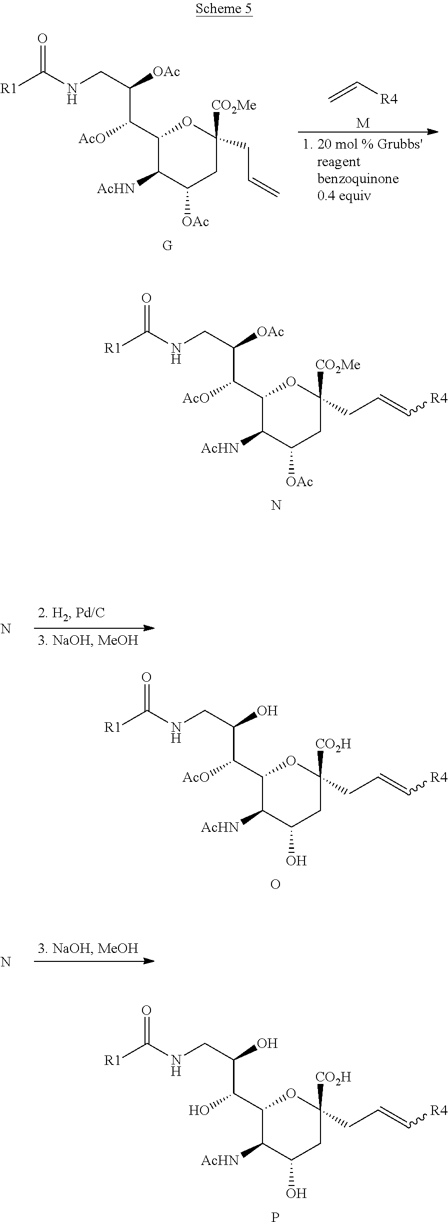

Additional C2-analogs of this invention can be produced via olefin metathesis chemistry using allyl compounds G in combination with compounds M with catalytic Grubbs' reagent, or modification of the Grubbs' reagent to form compounds N at high dilution as shown in Scheme 5. Compounds N can be reduced using palladium on carbon in the presence of hydrogen gas followed by hydrolysis of the protecting groups and C1-methylester to form analogs O. Likewise the protecting groups on compounds N can be hydrolyzed in the presence of hydroxide to form compounds P.

2. General Procedure for the Preparation of C2-styrene Analogs (Compound T) of Neuraminic Acid C2-C-glycosides, Option A.

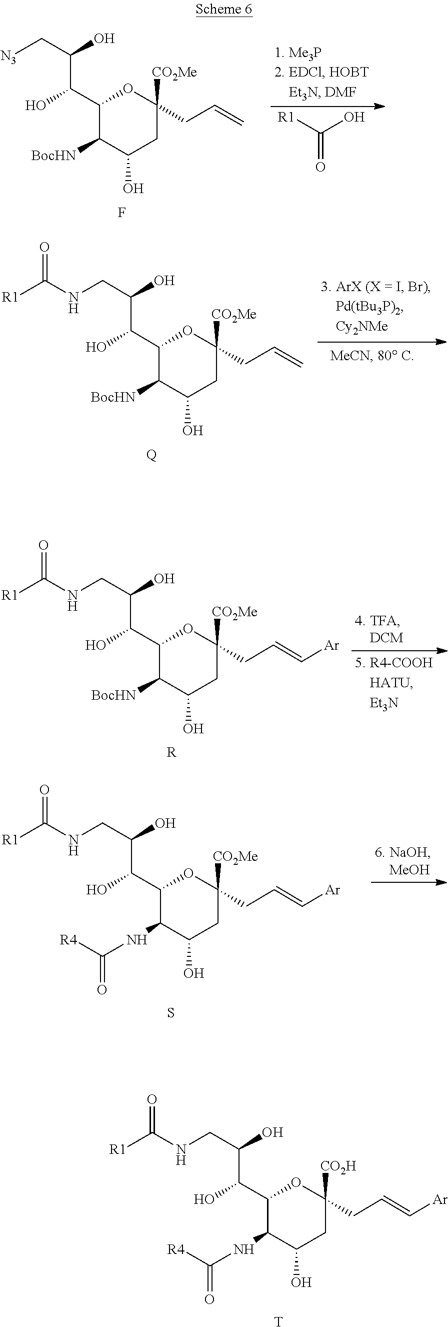

The C-2 styrene series of analogs were prepared starting from compound F (R=Boc) with functionalization at the C9-position, then the C2-allyl group, and finally the C5-amino group as shown in Scheme 6. As described previously the C9-azide was first reduced followed by acylation under mild conditions to provide compound Q. Arylation of the C-2 allyl at the terminal carbon under Heck coupling conditions provided the styrene intermediate R. Deprotection of the Boc-group at C5, followed by another acylation with various carboxylic acids to provide compounds S, and finally hydrolysis of the C1-ester provided analogs x-y of compound T with various substitution at R1, R4 and Ar. Detailed description for the production of these analogs is provided below.

3. General Procedure for the Preparation of C2-styrene Analogs (Compound T) of Neuraminic Acid C2-C-glycosides, Option B.

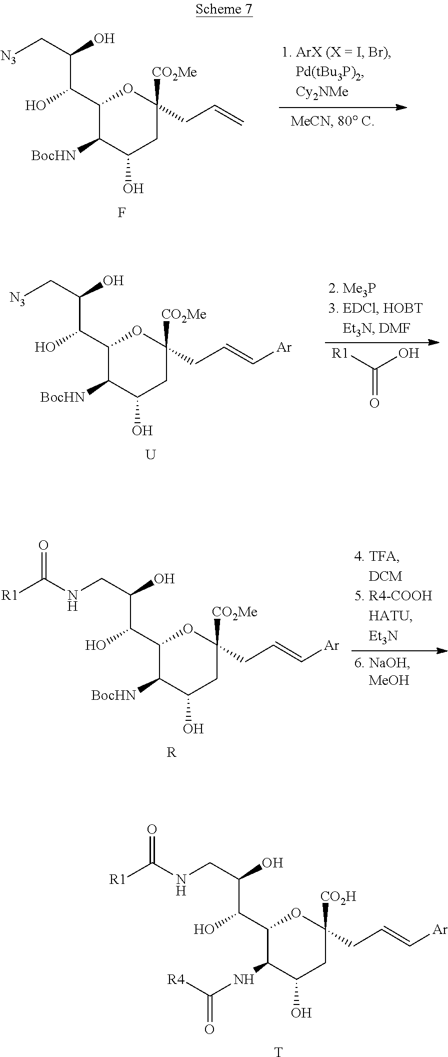

An alternative method to prepare the C-2 styrene series or compound T analogs starting from compound F (R=Boc) is to change the order of the sequence of steps as shown in Scheme 7. One first performs the Heck coupling reaction on the C2-allyl group with various aromatic halides to form intermediates U, then the sequence of steps to acylate the C-9 position, followed by the acylation processes at the C5-position, and finally hydrolysis of the C1 ester.

4. General Procedure for the Preparation of C2-styrene Analogs (Compound T) of Neuraminic Acid C2-C-glycosides, Option C.

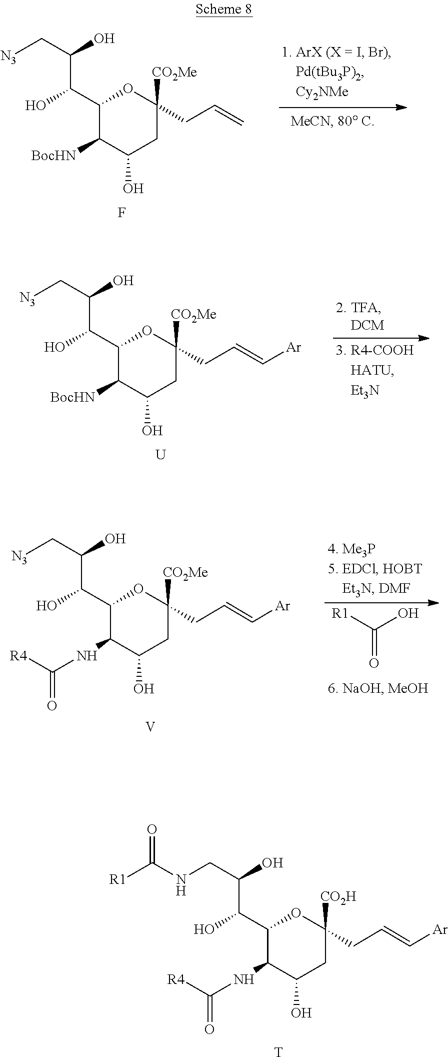

A third method to prepare the C-2 styrene series or compound L analogs starting from compound F (R=Boc) with functionalization is to change the order of the sequence of steps as shown in Scheme 8. One first performs the Heck coupling reaction on the C2-allyl group with various aromatic iodides, then the sequence of steps to acylate the C-5 position to form intermediates V, followed by the acylation process at the C9-position, and finally hydrolysis of the C1 ester.

5. General Procedure for the Preparation of C2-styrene Analogs (Compound T) of Neuraminic Acid C2-C-glycosides, Option D.

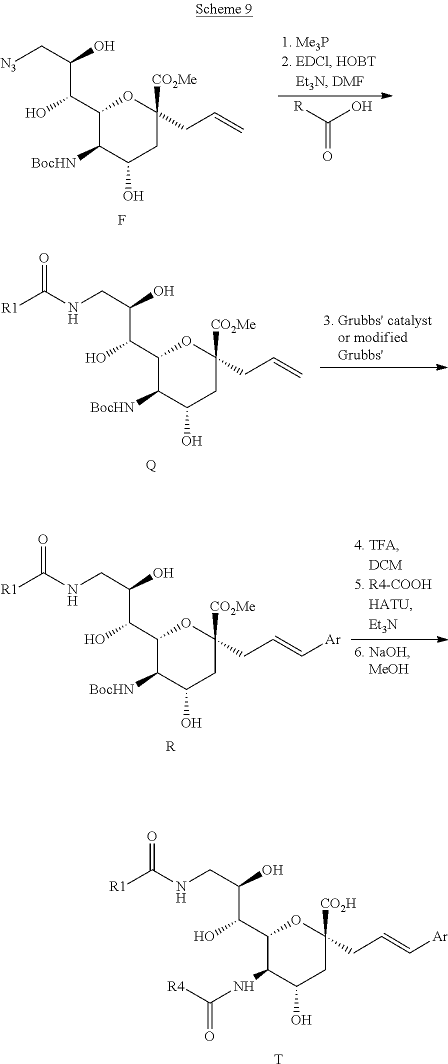

An alternative to the Heck-coupling reaction to generate various styrene-Ar analogs is to use Grubbs mixed metathesis reaction, or modifications of this reaction to form analogs of compound T when starting with intermediate F containing the C5-Boc-protected amine as exemplified in Scheme 9. Replacement of the Heck coupling reaction with the Grubbs' coupling reaction or a modification thereof can also be used in Scheme 7 and Scheme 8.

6. General Procedure for the Preparation of C2-dihydroxy and -dioxylane Analogs (Compounds Y, Z, AA, and BB) of Neuraminic Acid C2-C-glycosides.

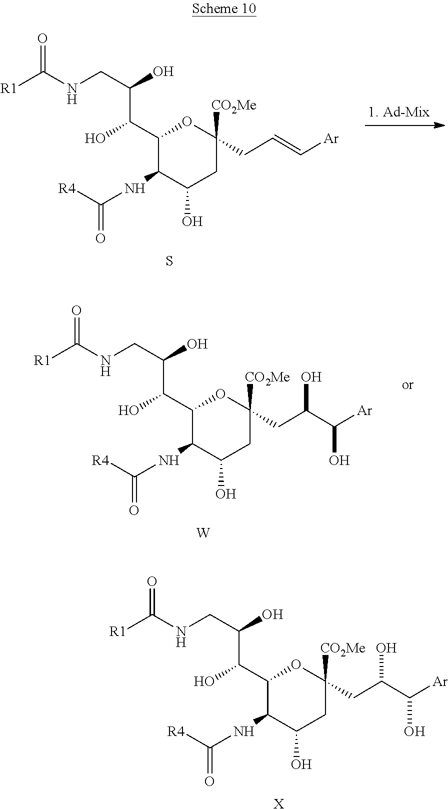

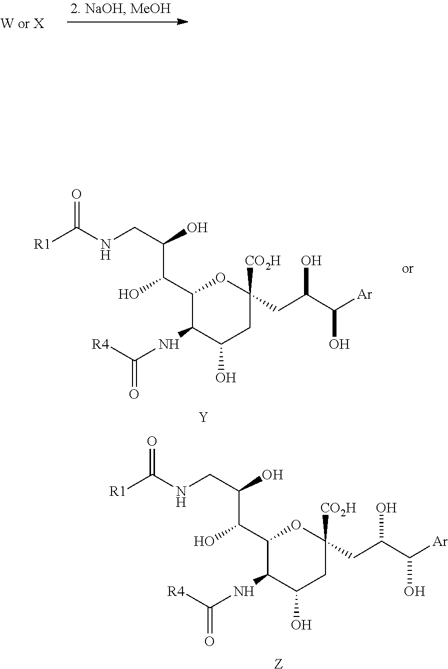

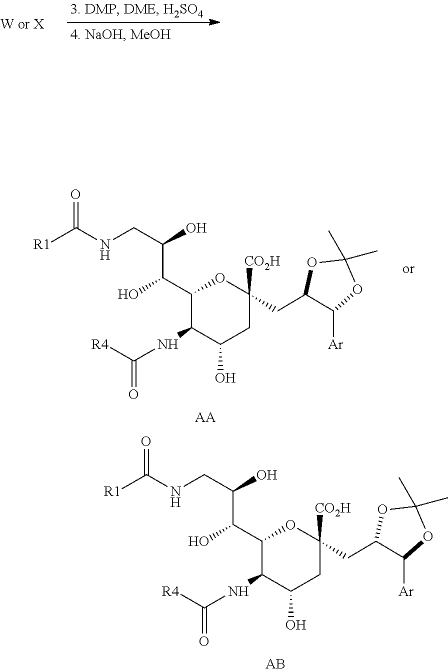

The C2-dihydroxy analogs can be generating starting with the styrene intermediate S, and using Sharpless' chiral hydroxylating conditions using AD-Mixα or AD-Mixβ to provide compounds W or X respectively as shown in Scheme 10. W or X can then be hydrolyzed in the presence of hydroxide to provide acid analogs Y or Z. The dioxalane analogs of W or X can be easily formed by using 2,2-dimethoxypropane in the presence of strong acid, followed by hydrolysis of the ester to provide compounds AA or BB respectively.

7. General Procedure for the Preparation of C2-O-glycoside analogs (compound AE) of Neuraminic Acid.

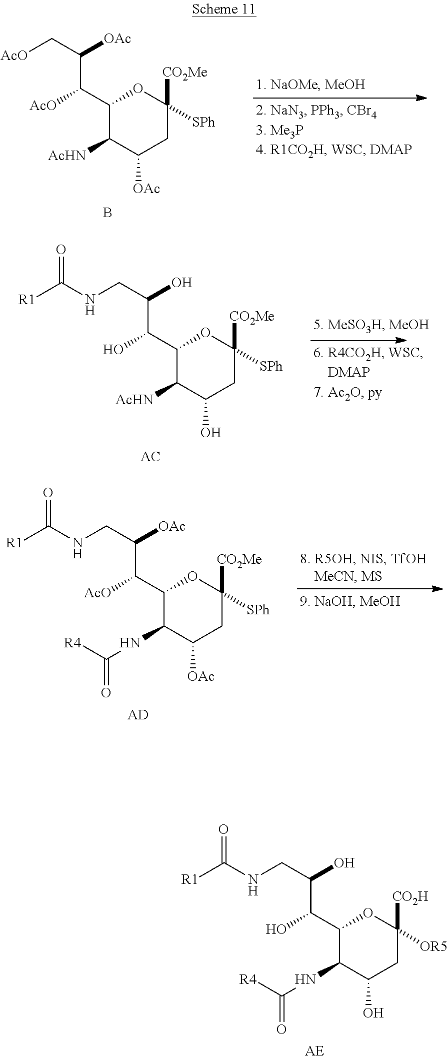

The C2-O-glycoside analogs of this invention can be generated using the synthetic method depicted in Scheme 11. Starting with intermediate B from Scheme 1, one generates the C9-acyl analogs by hydrolysis of the acyloxy-protecting groups using sodium methoxide, using Mitsunobu conditions to form the C9-azide, followed by reduction of the azide and selective acylation the resultant C9-amine to form intermediates AC. Hydrolysis of the N-acyl group on AC, followed by selective acylation of the C5-amino group, and then re-acylation of the free hydroxyl groups forms compounds AD. The key glycosidation of the C2-center using various alcohols with N-iodosuccinimide and tosyl acid under anhydrous conditions, followed by complete hydrolysis of the acyloxy-groups and methyl ester provides C2-O-glycosides AE.

### 8. General Procedure for the Preparation of Macrocyclic Analogs (Compound AN) of Neuraminic Acid.

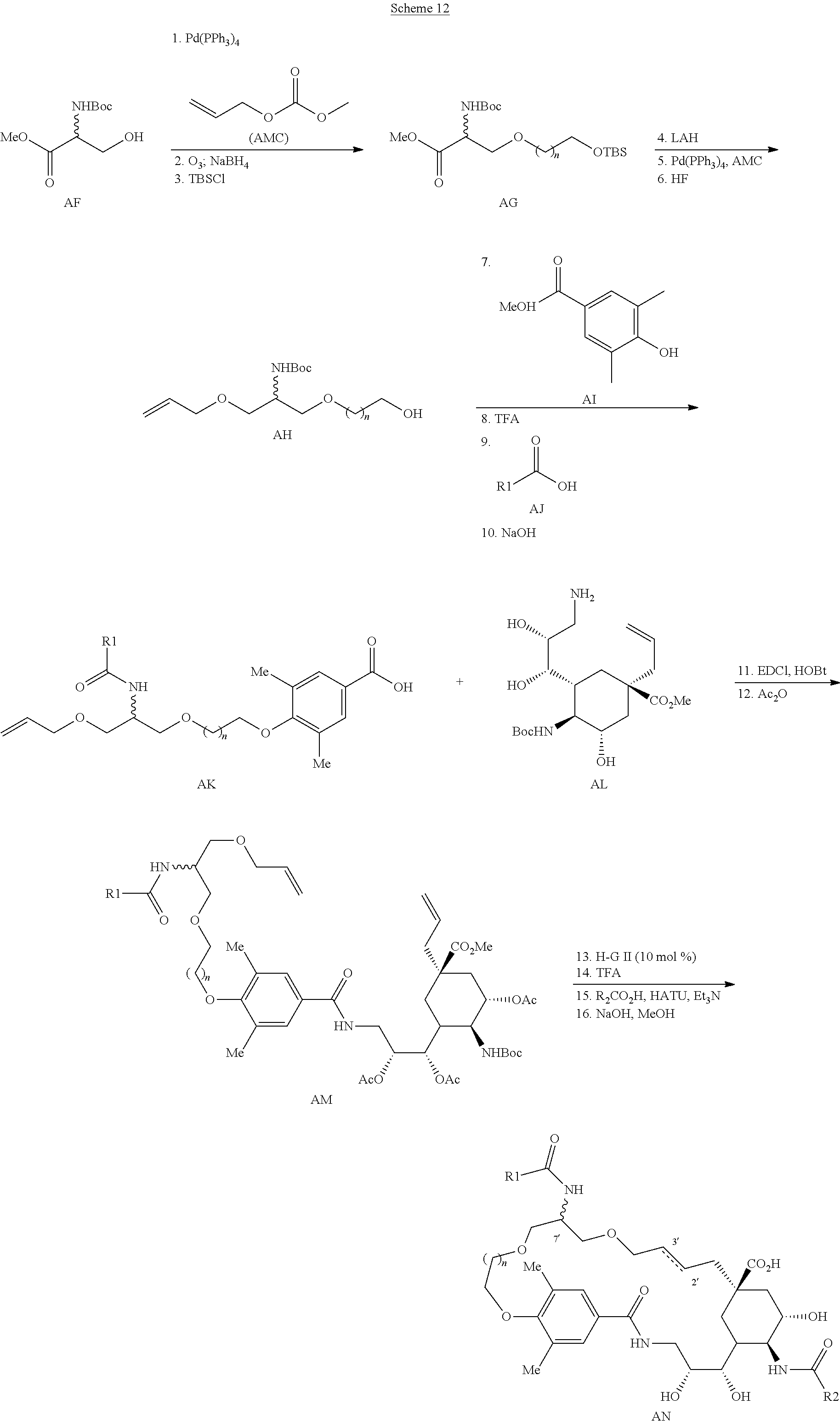

One example of a synthetic route to the macrocyclic series of compounds is shown in Scheme 12. Using either Boc protected D- or L-serine methyl ester (AF) as a starting material one can generate the protected allyl ether using Mitsunobu modified conditions, followed by ozone oxidation of the terminal olefin, reduction to the ensuing alcohol, and finally t-butyldimethylsilyl-protection to provide compound AG where n is equal to one. Reduction of the ester under standard hydride conditions, allylation using similar Mitsunobu conditions, and desilylation provides the primary alcohol AH (n=1). A commercially available phenol such as AT can then be coupled with AH using standard Mitsunobu conditions, followed by deprotection of the chiral amine, condensation with the appropriate acid AJ, and ester hydrolysis provides compound AK. AK is then condensed with the amine compound AL, generated from the reduction of the azide of compound F (Scheme 1, R=Boc), followed by acylation of all free hydroxyl groups to obtain the fully protected intermediate compound AM. Using the modified Hoyveda-Grubbs ring closing metasesis with AM followed by Boc-removal, condensation with a selected acid, and then final hydrolysis of the C-1 ester using hydroxide provides the desired macrocycle AN containing an olefin. The saturated analog of AN can be obtained using simple hydrogenation conditions.

### 9. Alternative Procedure for the Preparation of Macrocyclic Analogs (Compound AV) of Neuraminic Acid.

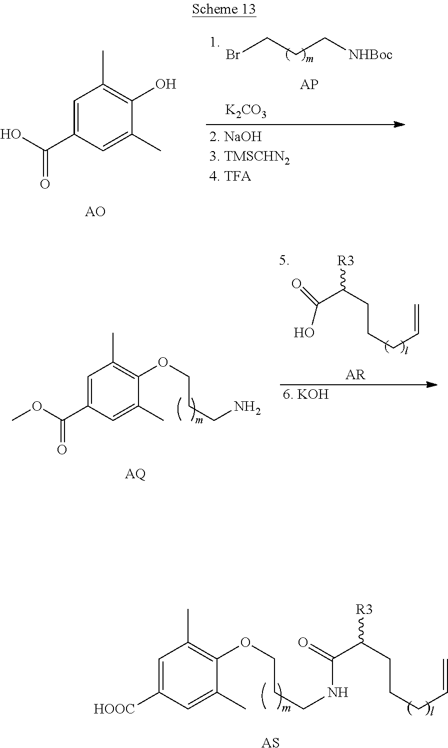

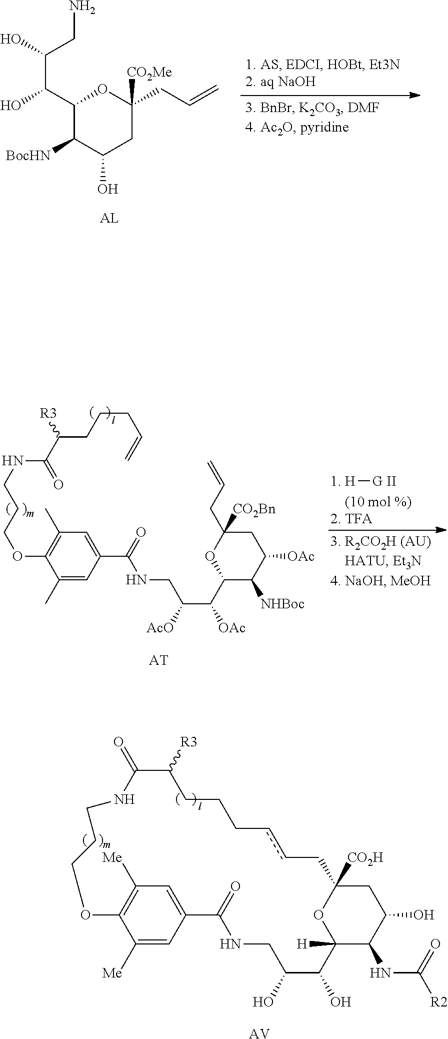

A similar approach for the generation of the macrocyclic compounds is shown in Scheme 13. Starting with the 4-hydroxybenzoic acid AO the amino ether compound AQ can be easily generated by first formation of the ether under mild basic conditions, hydrolysis of undesired ester formed from the ether formation reaction, addition of diazomethane to form the methyl ester, and Boc-deprotection of the terminal amine. Condensation of AQ with the appropriate ester containing a terminal olefin, AR, followed by ester hydrolysis provides compound AS. AS can then be condensed with AL followed by ester exchange to the benzyl ester, and then protection of the hydroxyl groups with acetyls to provide AT. Ring closing metasesis of AT followed by Boc-removal, condensation with a selected acid, AU, then final debenzylation using hydroxide provides the desired macrocycle AV containing an olefin. Final debenzylation using hydrogenolysis conditions provides the saturated macrocycle AV.

10. General Procedure for the Preparation of C1 Analogs (Compounds x-y) of Neuraminic Acid.

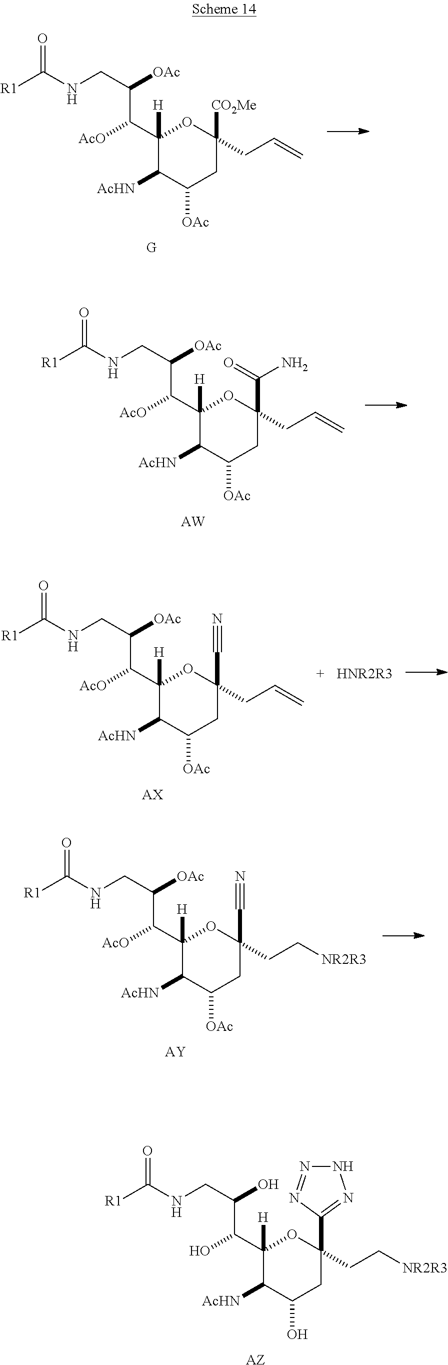

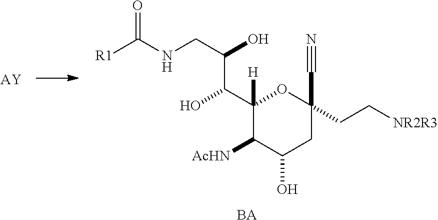

The preparation of the C1 analogs of this invention begins with the modification of key intermediate G to provide the primary amide, AW, which can then be dehydrated to form the nitrile, AX. Using the chemistry described in the general procedures 2 or 3, compound AY can be obtained.

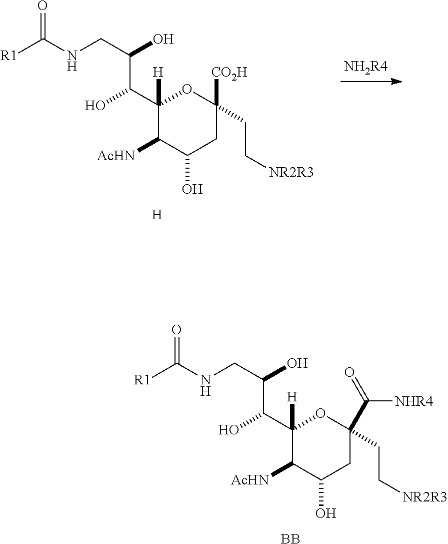

Various amide analogs for this series can be prepared from the previously described generic compound H being condensed with various amides, BB, using state of the art conditions.

Purification Methods used for Examples:

The tables below provide the typical HPLC protocols for the purification of the examples listed in the Table 3.

### Analytical Purification Methods

| Compound: | Acidic Conditions | | | | |
|---|---|---|---|---|---|
| Column: | ACQUITY UPLC CSH C18 Column \| 130 Å \| 1.7 μm \| 2.1 × 50 mm | | | | |
| Instrument: | Waters UPLC | | | | |
| Mobile phase A: | Water + 0.1% Formic Acid | | | | |
| Mobile phase B: | Acetonitrile + 0.1% Formic Acid | | | | |
| Column Temperature: | 40° C. | | | | |
| | TIME (min) | A % | B % | Curve | |
| Gradient: | 0 | 95 | 2 | X | Initial Conditions - Begin Gradient |
| | 1.9 | 90 | 95 | 6 | End Gradient - Begin Wash Step |
| | 2.4 | 70 | 95 | 6 | |
| | 2.5 | 5 | 2 | 6 | Reset Conditions |
| | 3.0 | 5 | 2 | 6 | |

| | |
|---|---|
| Flow Rate (mL/min): | 0.8 |
| Wavelength: | Diode array detector |

| Compound: | Basic Conditions | | | | |
|---|---|---|---|---|---|
| Column: | XBridge BEH C18 Column \| 130 Å \| 3.5 μm \| 3 × 50 mm | | | | |
| Instrument: | Waters UPLC | | | | |
| Mobile phase A: | Water + 0.1% Ammonium Hydroxide | | | | |
| Mobile phase B: | Acetonitrile + 0.1% Ammonium Hydroxide | | | | |
| Column Temperature: | 40° C. | | | | |
| | TIME (min) | A % | B % | Curve | |
| Gradient: | 0 | 95 | 5 | X | Initial Conditions - Begin Gradient |
| | 2.8 | 90 | 95 | 6 | End Gradient - Begin Wash Step |
| | 3.0 | 70 | 95 | 6 | |
| | 3.0 | 5 | 5 | 6 | Reset Conditions |
| | 4.0 | 5 | 5 | 6 | |

| | |
|---|---|
| Flow Rate (mL/min): | 1.5 |
| Wavelength: | Diode array detector |

### Preparative Purification Methods

| Compound: | Acidic Conditions | | | |
|---|---|---|---|---|
| Column: | Xselect CSH Prep C18, 5 μm OBD, 19 × 100 mm | | | |
| Instrument: | Agilent 1290 Infinity II preparative LC/MSD XT | | | |
| Mobile phase A: | Water + 0.1% Formic Acid | | | |
| Mobile phase B: | Acetonitrile + 0.1% Formic Acid | | | |
| Column Temperature: | Ambient | | | |
| Injection Vol. | 1-2 mL | | | |
| | TIME (min) | A % | B % | Notes |
| Gradient: | 0 | 95 | 5 | Starting Conditions & Isocratic Hold Start |
| | 1 | 95 | 5 | Isocratic Hold Stop & Gradient Start |
| | 28 | 5 | 95 | Gradient Stop & Column Wash Start |
| | 30 | 5 | 95 | Column Wash stop |
| | 30.1 | 95 | 5 | Reset column Conditions Start |
| | 31 | 95 | 5 | Method End |

| | |
|---|---|
| Flow Rate (mL/min): | 40 |
| Wavelength: | 214 nm, 260 nm, ELSD |

| Compound: | Basic Conditions | | | |
|---|---|---|---|---|
| Column: | XBridge Prep C18, 5 μm OBD, 19 × 100 mm | | | |
| Instrument: | Agilent 1290 Infinity II preparative LC/MSD XT | | | |
| Mobile phase A: | Water + 0.1% Ammonium Hydroxide | | | |
| Mobile phase B: | Acetonitrile + 0.1% Ammonium Hydroxide | | | |
| Column Temperature: | Ambient | | | |
| Injection Vol. | 1-2 mL | | | |
| | TIME (min) | A % | B % | Notes |
| Gradient: | 0 | 95 | 5 | Starting Conditions & Isocratic Hold Start |
| | 1 | 95 | 5 | Isocratic Hold Stop & Gradient Start |
| | 28 | 5 | 95 | Gradient Stop & Column Wash Start |
| | 30 | 5 | 95 | Column Wash stop |
| | 30.1 | 95 | 5 | Reset column Conditions Start |
| | 31 | 95 | 5 | Method End |

| | |
|---|---|
| Flow Rate (mL/min): | 40 |
| Wavelength: | 214 nm, 260 nm, ELSD |

### Preparation of A-001 and A-002

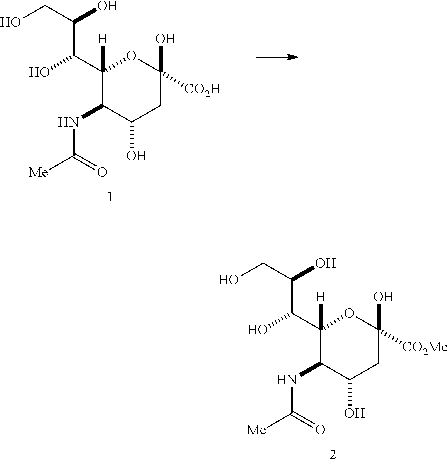

To a mechanically stirred suspension of (2S′,4S′,5R,6R)-5-acetamido-2,4-dehydroxy-6-((1R,2R)-1,2,3-trihydroxypropyl)tetrahydro-2H-pyran-2-carboxylic acid (1, 10.0 g, 32.3 mmol) in methyl alcohol (210 mL) was added Dowex 5w×4 (20 g) at room temperature. The reaction was stirred for 22 h after which time the suspension was filtered over Celite, washed with methanol (2×20 mL), and the resultant filtrate was concentrated and dried under vacuum. The crude product (2, 10.34 g, 32.0 mmol, 99%) was used in the next step. (MW_{Calc}+23=346.30; MW_{Obs}=346.14)

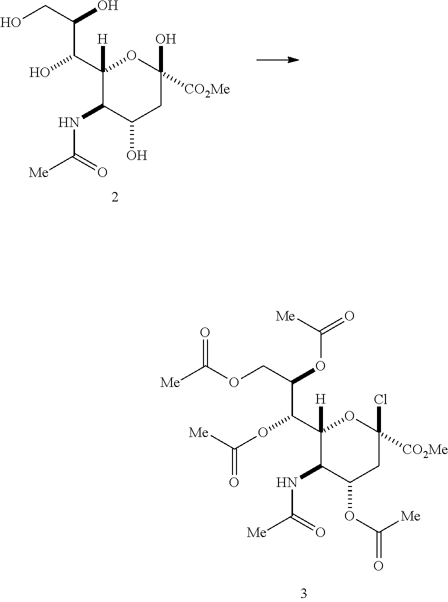

(2S,4S,5R,6R)-methyl 5-acetamido-2,4-dihydroxy-6-((1R,2R)-1,2,3-trihydroxypropyl)tetrahydro-2H-pyran-2-carboxylate (2, 10.34 g, 31.98 mmol) in acetyl chloride (300 ml, 4.22 mol) was mechanically stirred at 35° C. for 2 d. The completed clear reaction was concentrated in vacuo and azeotroped with toluene (2×100 ml ea). The residue was dissolved in MTBE (100 mL) with heating and treated with n-heptane (50 mL). More MTBE (20 mL) was added to dissolve oily precipitate. The mixture was stirred at 65° C. (bath) for 20 min, at 45° C. for (2 h) and at rt for 20 h (no precipitate). The mixture was concentrated in vacuo to obtain crude product (3, 15.58 g, 30.6 mmol, 96%) that was used in the next step. (MW_{Calc}+H=510.8890; MW_{Obs}=510.10)

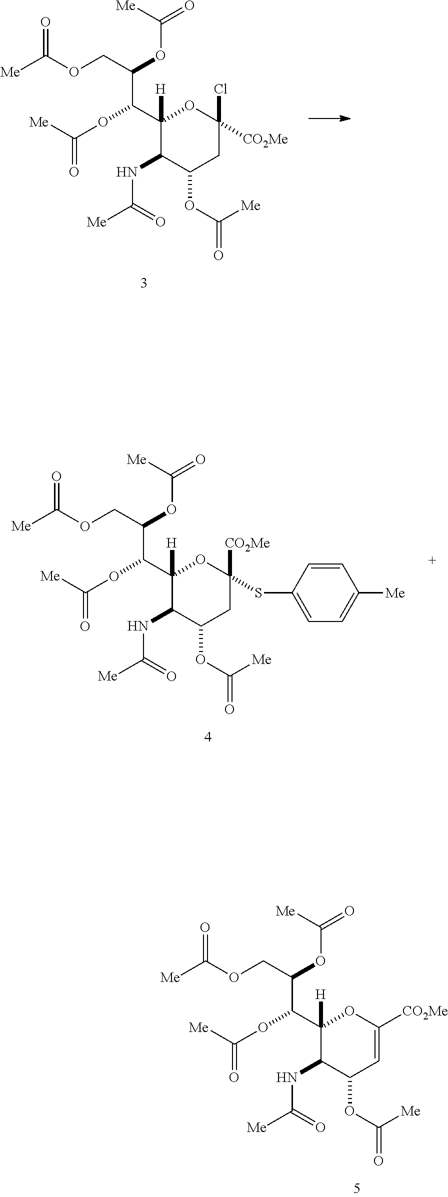

T To a stirred solution of (1S,2R)-1-((2R,3R,4S,6R)-3-acetamido-4-acetoxy-6-chloro-6-(methoxycarbonyl)tetrahydro-2H-pyran-2-yl)propane-1,2,3-triyl triacetate (3, 21 g, 41.19 mmol) and p-toluenethiol (15.35 g, 123.56 mmol) in DCM (315 ml) at 0° C. was added dropwise Hunig's Base (23.74 ml, 135.91 mmol) over a 20 min period maintaining the temperature at 0° C. The reaction was slowly allowed to warm to RT over a 16-h period. The completed reaction was poured over sat. sodium bicarbonate (900 mL), back extracted with ethyl acetate (600 mL), washed with 50% brine, and dried over anhydrous Na₂SO₄. Concentrated. The crude product was purified over a SNAP Ultra HP 340 g silica gel column in 5% ethyl acetate in n-heptane and eluted with 5% ethyl acetate in n-heptane (1CV), 0% to 100% ethyl acetate in n-heptane (1° C. V), and 100% ethyl acetate (2CV). Obtained compound 4 (20.36 g, 24.1 mmol, 83%) (MW_{Calc}+Na=620.63; MW_{Obs}=620.20). Compound 5 was found to be the major impurity.

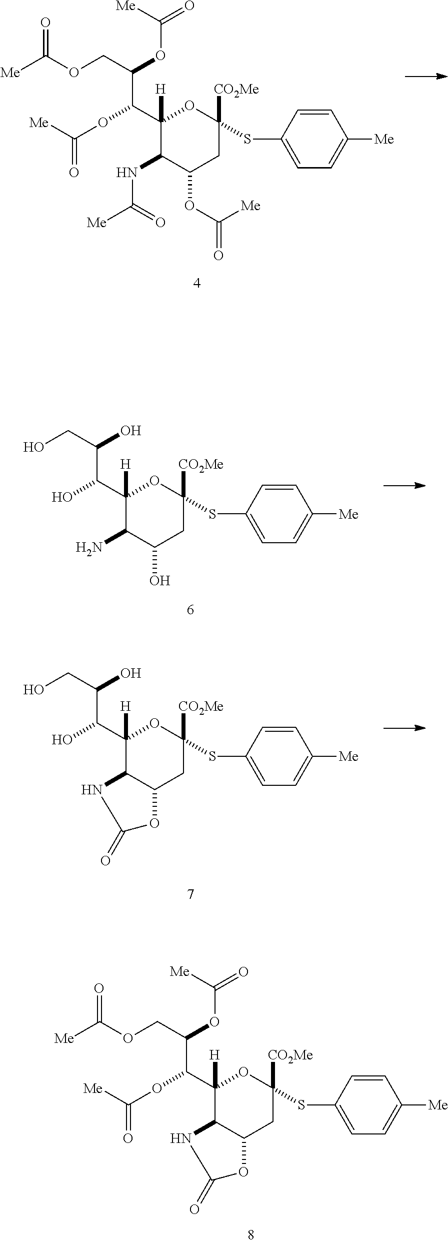

To a stirred solution of (1S,2R)-1-((2R,3R,4S)-3-acetamido-4-acetoxy-6-(methoxycarbonyl)-6-(phenylthio)tetrahydro-2H-pyran-2-yl)propane-1,2,3-triyl triacetate (4, 17 g, 28.44 mmol) in methanol (100 ml) was added methanesulfonic acid (9.24 mL, 142.23 mmol) followed warming to 65° C. and stirring overnight. Triethylamine (2.472 ml, 17.735 mmol) was added slowly to near completed reaction followed concentration and azeotroping to dry with acetonitrile (3×100 mL ea.). The resulting crude residue, 6, was dried under high vacuum overnight and used in the next reaction. (MW_{Calc}+H=388.45; MW_{Obs}=388.11).

To a stirred solution of (4S,5R,6R)-methyl 5-amino-4-hydroxy-2-(p-tolylthio)-6-((1R,2R)-1,2,3-trihydroxypropyl)tetrahydro-2H-pyran-2-carboxylate (6, 40 g, 25.81 mmol) in acetonitrile (150 mL) and water (150 mL) at 0° C. was added sodium bicarbonate (10.84 g, 129.05 mmol) followed by a stepwise addition of 4-nitrophenyl carbonochloridate (13.01 g, 64.53 mmol) in acetonitrile (150 mL) over 30-min period keeping the temperature between 0-5° C. The reaction mixture was stirred for an additional 2.5 h at 0° C. after which time it was diluted with ethyl acetate (500 mL) and the layers were separated. The aqueous layer was extracted with ethyl acetate (100 mL ea), and the combined organic layers were washed with brine (100 mL) followed by concentration of the organic layer and vacuum drying to provide the yellow solid 7 (28.0 g, crude mixture). (MW_{Calc}+H=414.44; MW_{Obs}=414.08).

To a stirred solution of crude (3aR,4R,6S,7aS)-methyl 2-oxo-6-(p-tolylthio)-4-((1R,2R)-1,2,3-trihydroxypropyl)hexahydro-2H-pyrano[3,4-d]oxazole-6-carboxylate (7, 27 g, 21.551 mmol) in pyridine (29.6 ml) under a N₂ atmosphere at 5° C. was dropwise acetic anhydride (30.5 ml, 323.26 mmol) over a 30 minute period. The reaction mixture was allowed to warm to RT and stirred for 19 h. The complete reaction was diluted with ethyl acetate (500 mL) followed by 2 N aqueous HCl (500 mL), and transferred into a separatory funnel with additional ethyl acetate (200 mL). The layers were separated, and the organic layer was washed with 1N HCl (100 mL), sat. ammonium chloride (100 mL) and brine (100 mL). The combined aqueous layers were extracted with ethyl acetate (1×300 mL), and the resultant organic layer was washed with brine (50 mL). The combined organic layers were concentrated followed by purification of the crude residue over SNAP 340 g cartridge of silica gel eluting with from 0-100% EtOAc heptane (10 cv transition). The desired product was collected, concentrated and dried under vacuum to provide the desired product 8 (12.2 g, 22.43 mmol, 104% w/solvent) as a foam. (MW_{Calc}+23=562.55; MW_{Obs}=562.07)

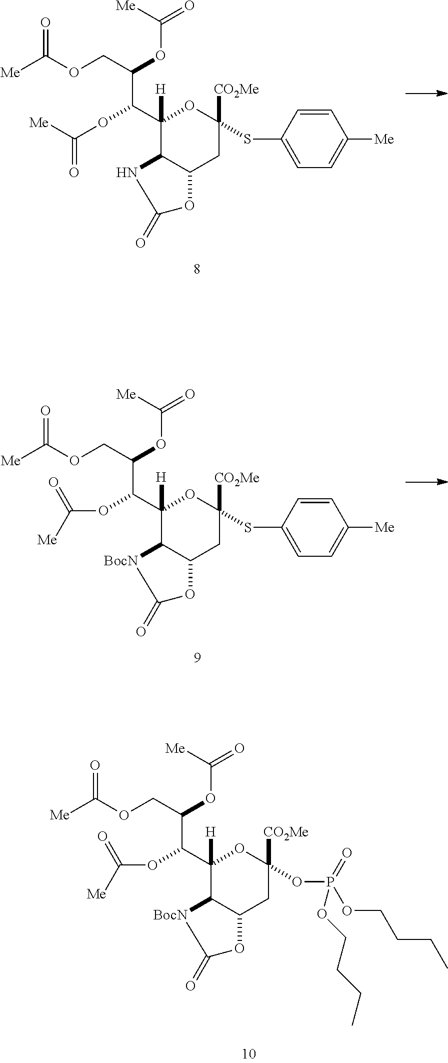

To a stirred solution of (1S,2R)-1-((3aR,4R,6S,7aS)-6-(methoxycarbonyl)-2-oxo-6-(p-tolylthio)hexahydro-2H-pyrano[3,4-d]oxazol-4-yl)propane-1,2,3-triyl triacetate (8, 9.0 g, 16.68 mmol) in THF (315 mL) was added BOC-anhydride (7.75 ml, 33.36 mmol) followed by DMAP (1.019 g, 8.34 mmol) at room temperature. The mixture was stirred for 30 m after which time the completed reaction was partially concentrated to approximately 30 mL and apply to SNAP silica gel column (100 g) eluting with 0-15% ethyl acetate in heptane (5 CV) then 15-100% ethyl acetate in heptane (10 CV). The fractions containing desire product were concentrate and high vacuumed to dryness to provide 9 (8.6 g, 13.44 mmol, 81%). (MW_{Calc}+23=662.67; MW_{Obs}=662.12).

To a stirred solution of dibutyl phosphate (7.76 ml, 41.70 mmol) and (3aR,4R,6S,7aS)-3-tert-butyl 6-methyl 2-oxo-6-(p-tolylthio)-4-((1S,2R)-1,2,3-triacetoxypropyl)tetrahydro-2H-pyrano[3,4-d]oxazole-3,6(6H)-dicarboxylate (9, 8.6 g, 0.445 mmol) in dry DCM (188 ml) was added dry 4A molecular sieves (2 g/mmol reagent) followed by stirring for 2 h. The mixture was cooled to 0° C. followed by the addition of N-iodosuccinimide (6.38 g, 28.36 mmol) followed by trifluoromethanesulfonic acid (0.25 mL, 2.84 mmol) in DCM (0.1 mL). The final reaction mixture was stirred at 0° C. for 4-5 hours after which time it was quenched with the addition of sodium thiosulfate (10 g) in NaHCO₃ (50 mL) and water (50 mL). EtOAc (100 mL) was added followed by stirring for an additional 5 m. The quenched suspension was filtered followed by separating the layers, and extracting the aqueous layer with ethyl acetate (2×50 mL ea). The combined organic layers were washed with sat. NaHCO₃ (5 mL) followed by brine (5 mL), dried over anhyd. Na2SO4, filtered and concentrated to dry. The crude product was purified over a Biotage SNAP silica gel column (100 g) column eluting with a gradient of 0-100% ethyl acetate in heptane (10 CV total) to obtain the desired product 10 (9.20 g, 12.68 mmol, 76%) after collection of the desired fractions, concentration and high vacuum to dryness. (MW_{Calc}+H=726.68; MW_{Obs}=726.30)

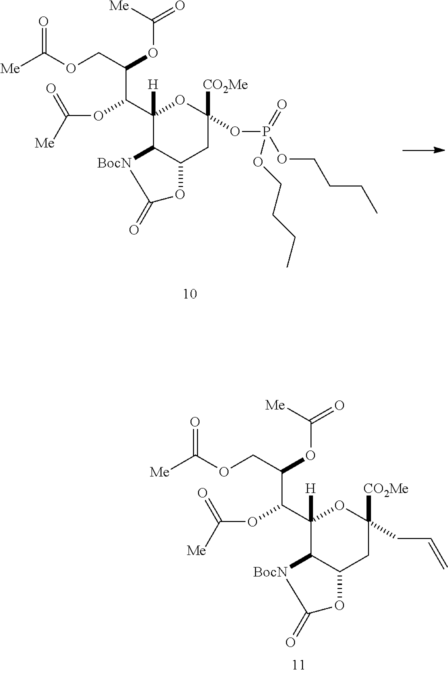

To a stirred solution of (3aR,4R,6S,7aS)-3-tert-butyl 6-methyl 6-((dibutoxyphosphoryl)oxy)-2-oxo-4-((1S,2R)-1,2,3-triacetoxypropyl)tetrahydro-2H-pyrano[3,4-d]oxazole-3,6(6H)-dicarboxylate (10, 7.7 g, 10.61 mmol) in DCM (193 ml) under a N₂ atmosphere was added dry 4A molecular sieves (10.6 g) followed by allyltributyltin (16.45 ml, 53.05 mmol). The mixture was stirred for 1 h at room temperature, and then cooled to −78° C. After a 10-minute period trimethylsilyltriflate (1.92 ml, 10.61 mmol) was added dropwise over a 5 min period, stirred for 1 h at −78° C., warmed to −55° C. for 5 min, and then cooled again to −78° C. The completed reaction was quenched with sat NaHCO₃ at −78° C. and allowed to warm to RT. The mixture was filtered, and the filter pad rinsed with ethyl acetate (3×50 mL ea). The layers were separated, and the organic layers was washed with brine, dried over sodium sulfate, filtered and concentrated under vacuum. Purification over a Biotage SNAP silica gel (100 g) eluting with a gradient of 0-75% EtOAc in heptane (10 CVs) provided 11 (4.05 g, 7.26 mmol, 68.5%). (MW_{Calc}+23=580.55; MW_{Obs}=580.25)

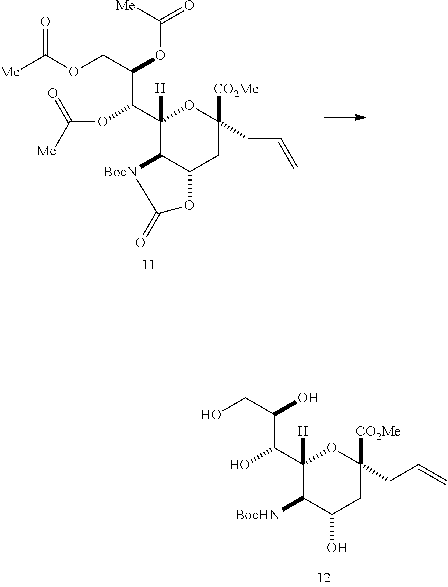

To a stirred solution of (3aR,4R,6R,7aS)-3-tert-butyl 6-methyl 6-allyl-2-oxo-4-((1S,2R)-1,2,3-triacetoxypropyl)tetrahydro-2H-pyrano[3,4-d]oxazole-3,6(6H)-dicarboxylate (11, 0.64 g, 1.15 mmol) in pure methanol (26 ml) was added slowly 25% sodium methoxide in methanol (0.67 mL, 2.96 mmol) over 2-3 minute period. The reaction was stirred for 30 m at room temperature after which time Amberlyst 15 resin (2 g) was added followed by stirring for an additional 5 minutes. The completed reaction was filtered over Celite, rinsed with methanol (2×20 mL), concentrated, and vacuum to dry to provide crude compounds 12 (0.45 g, 1.11 mmol, 97%). (MW_{Calc}+Na=428.44; MW_{Obs}=428.22)

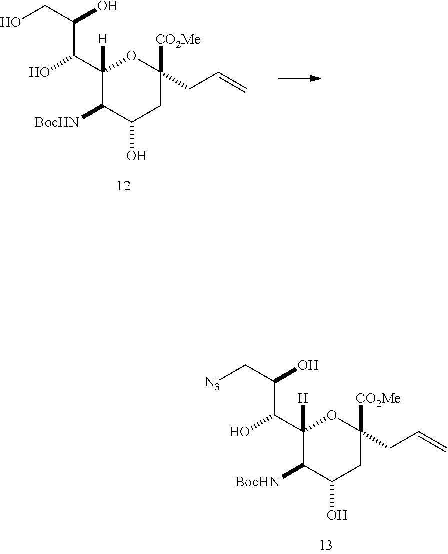

To a stirred solution of (2R,4S,5R,6R)-methyl 2-allyl-5-((tert-butoxycarbonyl)amino)-4-hydroxy-6-((1R,2R)-1,2,3-trihydroxypropyl)tetrahydro-2H-pyran-2-carboxylate (12, 1.45 g, 3.58 mmol) in DMF (18.9 mL) under a N2 atmosphere was added at room temperature sodium azide (1.16 g, 17.88 mmol) followed by triphenylphosphine (1.27 g, 4.83 mmol), and carbon tetrabromide (3.26 g, 9.84 mmol). The reaction mixture was stirred for 16 h. Additional sodium azide (1.16 g, 17.88 mmol) was added to the reaction, which was allowed to stir for 2 days. The completed reaction was diluted with water (100 mL) and extracted with ethyl acetate (3×30 mL ea). The combined organic layers were washed with brine (30 mL) and concentrated to dryness. The crude product was purified over a Biotage SNAP silica gel column (10 g) column eluting with a gradient of 30-100% ethyl acetate in heptane (10 CV total) to obtain the desired product 13 (1.20 g, 2.79 mmol, 78%) after collection of the desired fractions, concentration and high vacuum to dryness. (MW_{Calc}+Na=453.46; MW_{Obs}=453.20)

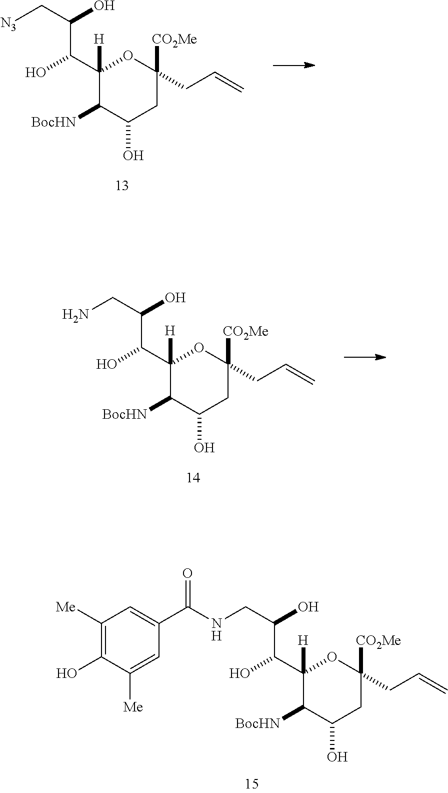

To a stirred solution of (2R,4S,5R,6R)-methyl 2-allyl-6-((1R,2R)-3-azido-1,2-dihydroxypropyl)-5-((tert-butoxycarbonyl)amino)-4-hydroxytetrahydro-2H-pyran-2-carboxylate (13, 0.68 g, 1.57 mmol) in THF (17 ml) and water (1.1 ml) at 0° C. was added 1N trimethylphosphine (4.71 ml, 4.71 mmol) followed by allowing to reaction to warm to room temperature, and stir for 16 h. The completed reaction was concentrated and azeotroped to dry with toluene (2×40 mL ea) to provide crude 14. (MW_{Calc}+H=405.46; MW_{Obs}=405.28).

To a stirred solution of (2R,4S,5R,6R)-methyl 2-allyl-6-((1R,2R)-3-amino-1,2-dihydroxypropyl)-5-((tert-butoxycarbonyl)amino)-4-hydroxytetrahydro-2H-pyran-2-carboxylate (14, 0.68 g, 1.57 mmol) in acetonitrile (6.76 ml) under a N₂ atmosphere at room temperature was added 4-Hydroxy-3,5-dimethylbenzoic acid (0.326 g, 1.96 mmol), and TEA (0.44 ml, 3.14 mmol) followed by N-(3-Dimethylaminopropyl)-N′-ethylcarbodiimide (0.488 g, 3.141 mmol). The reaction mixture was stirred for 16 h after which time ethyl acetate (50 mL) was added followed by washing with water (20 mL) and brine (20 mL). The organic layer was dried over Na₂SO₄, concentrated, and purified over a Biotage SNAP silica gel column (25 g) eluting with 40 to 100% ethyl acetate in heptane (10 CV), 0% to 20% MeOH in DCM (5 CV), and 20% MeOH in DCM (2 CV). The desired fractions were concentrated and high vacuum to dryness providing compound 15 (0.444 g, 0.803 mmol, 51.2%) as a white solid. (MW_{Calc}+Na=575.62; MW_{Obs}=575.20)

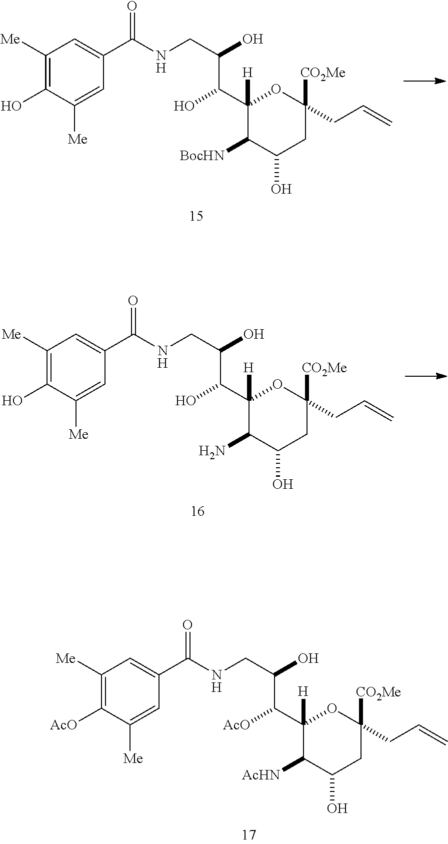

To a slurry solution of (2R,4S,5R,6R)-methyl 2-allyl-5-((tert-butoxycarbonyl)amino)-6-((1R,2R)-1,2-dihydroxy-3-(4-hydroxy-3,5-dimethylbenzamido)propyl)-4-hydroxytetrahydro-2H-pyran-2-carboxylate (15, 14.4 g, 26.06 mmol) in methylene chloride (50 mL) at 0-5° C. was added dropwise TFA (50 mL) over a 15-min period. The resulting solution was warmed to room temperature and stirred for an additional 30 min. The completed reaction was concentrated to dryness followed by azetroping to dryness with toluene (3×50 mL ea) to provide crude 16 as the TFA salt. (MW_{Calc}+H=453.22; MW_{Obs}=453.16).

To a stirred solution of 16 in pyridine at 0° C. was slowly added acetic anhydride (14.75 mL, 156.35 mmol) maintaining the temperature below 5° C. After the final addition, the reaction was warmed to room temperature, and stirred for an additional 14 h. The completed reaction was diluted with ethyl acetate (200 mL), washed with 0.5N HCl (50 mL) and brine (50 mL). The organic layer was dried over Na₂SO₄, concentrated, and purified over a Biotage SNAP silica gel column (200 g) eluting with 0-100% EtOAc in heptane to provide desired amide 17 (12.0 g 18.11 mmol, 70%) after concentration of the desired fractions and drying under vacuum. (MW_{Calc}+H=663.69; MW_{Obs}=663.14)

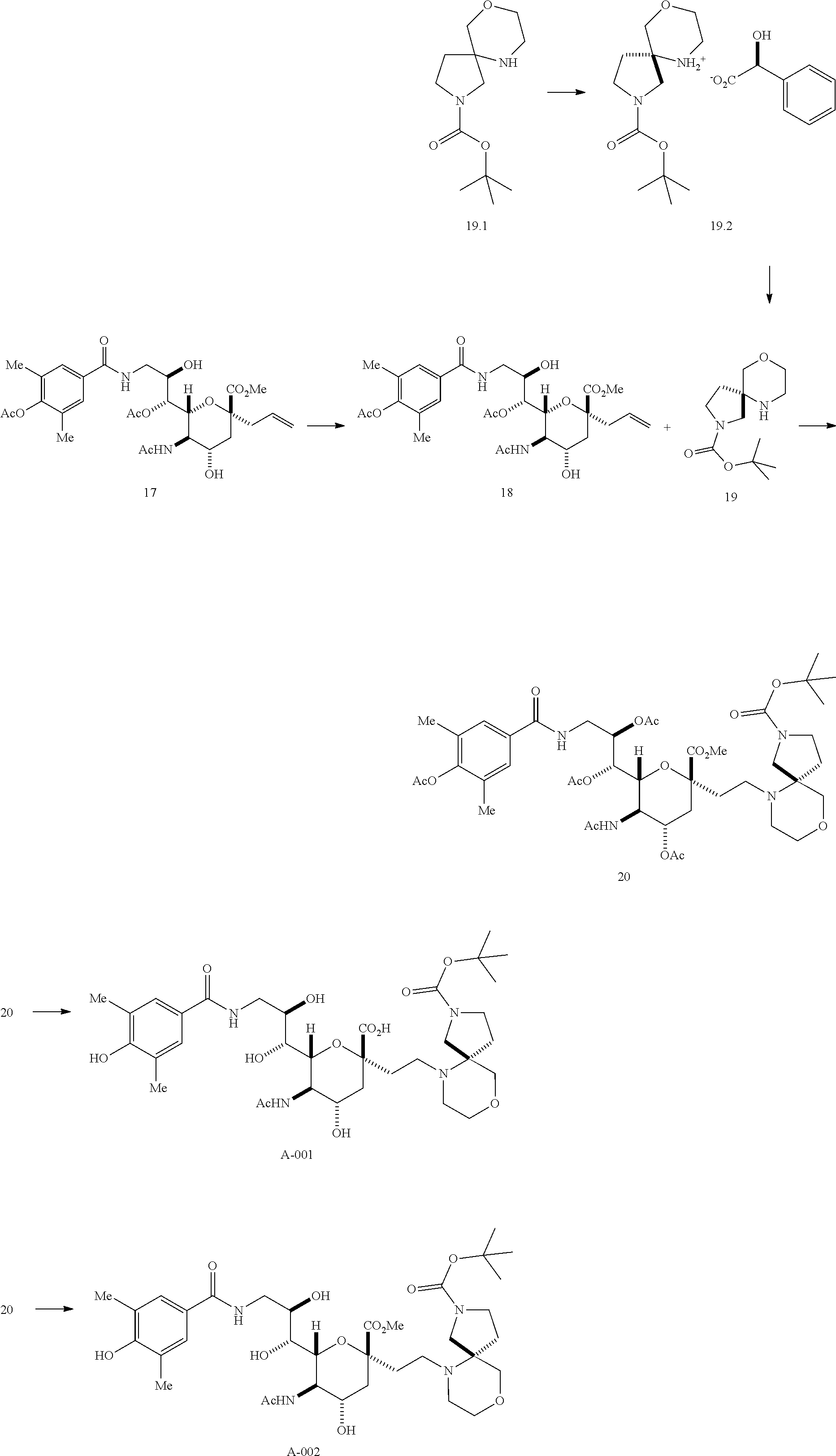

To a mechanical stirrer stirred solution of commercially available racemic tert-butyl 9-oxa-2,6-diazaspiro[4.5]decane-2-carboxylate (19.1, 1.2 g, 4.952 mmol) in toluene (9.6 mL) and 2-propanol (2.4 mL) at room temperature was added ((S)-(+)-mandelic acid (1.206 g, 7.923 mmol). The mixture was stirred at rt until crystallization occurred (ca 30 minutes) after which time the mixture was stirred for an additional 30 minutes. The resulting slurry was heated to 75° C. and stirred for 15 minutes followed by a slow cooling to 0° C. at an approximate rate of 25° C./hr (total ˜3 hours). The resulting crystals were filtered and rinsed with cold mixture of 9:2 toluene/IPA (5×5 mL ea). The solid was rinsed with heptane (5 mL), and then dried under vacuum for 24 h to provide compound 19.2 (889 mg, 2.253 mmol, 46%, 99% ee). The ee % was determined by the use of an Agilent 1100/CAD with a ChiralPak IA, 4.6×250 mm #TE-030 using a mobile phase of 80% n-heptane with 0.1% diethyl amine, and 20% 1:1 mix of methanol:ethanol with 0.1% DEA. A flowrate of 1 mL/minute, UV detection at 214 nm, column temperature at 35° C.

To a stirred slurry of tert-butyl (S)-9-oxa-2,6-diazaspiro[4.5]decane-2-carboxylate (S)-2-hydroxy-2-phenylacetate salt (19.2, 1.58 g, 4.005 mmol) in dichloromethane (100 mL) was add saturated aqueous sodium bicarbonate (100 mL). The mixture was shaken for 5 min followed by separation of the layers. The organic layer was washed a second time with saturated aqueous sodium bicarbonate. The combined aqueous layers were extracted with DCM (2×25 mL). The combined organic phases were concentrated under reduced pressure, and azeotroped to dry with dichloroethane (3×50 mL ea) to provide compound 19 (0.97 g, 4.01 mmol, 100%), which was used in the next steps without further purification.

To a stirred solution of (1R,2R)-1-((2R,3R,4S,6R)-3-acetamido-4-acetoxy-6-allyl-6-(methoxycarbonyl)tetrahydro-2H-pyran-2-yl)-3-(4-acetoxy-3,5-dimethylbenzamido)propane-1,2-diyl diacetate (17, 1.30 g, 1.96 mmol) in 1,4-dioxane (23.4 mL) and water (7.80 ml) was added 2,6-lutidine (0.46 ml, 3.923 mmol), osmium tetroxide (0.25 mL, 0.039 mmol), and sodium periodate (1.678 g, 7.847 mmol) at room temperature. The completed reaction was diluted with ethyl acetate (100 mL), washed with sat sodium bicarbonate (50 mL), and with brine (50 mL). The organic layer was dried over Na₂SO₄ and concentrated to dryness to provide the crude aldehyde, 18.

To a stirred solution of 18 in dichloroethane (19.5 mL) and MeOH (3.90 ml, 96.398 mmol) was added at room temperature tert-butyl (S)-9-oxa-2,6-diazaspiro[4.5]decane-2-carboxylate (19, 0.523 g, 2.158 mmol) as a salt with (S)-mandelic acid, acetic acid (0.786 ml, 13.73 mmol) and 4 AMS (3 g). The suspension was stirred for 1 h followed by the addition of sodium triacetoxyborohydride (0.832 g, 3.923 mmol) and stirring for an additional 16 h. The completed reaction was quenched with saturated NaHCO₃ (10 mL) and extracted with EtOAc (3×15 mL ea). The combined organic layers were dried over Na₂SO₄ and concentrated to dryness to provide the crude amine, 20. (MW_{Calc}+H=891.42; MW_{Obs}=891.55).

To a stirred solution of (1R,2R)-1-((2R,3R,4S,6R)-3-acetamido-4-acetoxy-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)-3-(4-acetoxy-3,5-dimethylbenzamido)propane-1,2-diyl diacetate (20, 20.3 mg, 0.023 mmol) in 7 N ammonia in methanol (2 mL, 14.00 mmol) at room temperature was sealed and placed in microwave apparatus at 120° C. for 1 h. The completed reaction was cooled to room temperature and purified directly by HPLC to provide A-002 (8.5 mg, 0.012 mmol, 51%) after collection of the desired fractions, concentration, and drying under vacuum. (MW_{Calc}+H=723.37; MW_{Obs}=723.54).

To a stirred solution of 20 methanol (3.9 ml) and THF (15.6 ml) at room temperature was added 1 N sodium hydroxide in water (19.62 ml, 19.62 mmol). The reaction was stirred for 48 h after which time the completed reaction was diluted with methanol (25 mL) an injected directly in aliquots of 0.05 mL onto a Waters Sunfire Prep C18 column (5 μm, 10×250 mm) eluting with a gradient of 90:10 to 60:40 water (containing 0.1% formic acid) to acetonitrile over 8 min, 60:40 to 1:99 water (containing 0.1% formic acid) to acetonitrile over 1 min, 1:99 water (containing 0.1% formic acid) to acetonitrile for 1 min, followed by re-charging the solvent conditions on the column to the original 90:10 water (containing 0.1% formic acid) to acetonitrile for 4.5 min prior to injection of the next aliquot. Concentration of the desired fractions to dryness followed by azeotroping with toluene (3×5 mL ea.) and drying under high vacuum provide compound A-001 (0.60 g, 0.846 mmol, 43.2%). (MW_{Calc}+H=709.81; MW_{Obs}=709.62).

### Preparation of A-003 to A-026

A-003 was prepared in a similar fashion to A-001 starting with compound 18 (137.0 mg, 0.206 mmol) and commercially available (R)-tert-butyl 9-oxa-2,6-diazaspiro[4.5]decane-2-carboxylate (82 mg, 0.340 mmol; which was obtained by separation of each enantiomer using chiral HPLC Method X from the commercially available racemic mixture) to provide A-003 (37.7 mg, 0.053 mmol, 26% overall yield). (MW_{Calc}+H=709.36; MW_{Obs}=709.49).

A-004 was prepared in a similar fashion to A-001 starting with compound 18 (30.0 mg, 0.045 mmol) and commercially available tert-butyl (5S,8R)-8-methyl-9-oxa-2,6-diazaspiro[4.5]decane-2-carboxylate (18.5 mg, 0.072 mmol) to provide A-004 (11.8 mg, 0.016 mmol, 36% overall yield). (MS_{Calc}+H=723.37; MW_{Obs}=723.24).

A-005 was prepared in a similar fashion to A-001 starting with compound 18 (30.0 mg, 0.045 mmol) and commercially available tert-butyl (5S,8S)-8-methyl-9-oxa-2,6-diazaspiro[4.5]decane-2-carboxylate (18.5 mg, 0.072 mmol) to provide A-005 (6.0 mg, 0.008 mmol, 18% overall yield). (MS_{Calc}+H=723.37; MW_{Obs}=723.25).

A-006 was prepared in a similar fashion to A-001 starting with compound 18 (50.0 mg, 0.075 mmol) and commercially available tert-butyl 1,8-diazaspiro[5.5]undecane-8-carboxylate (31.6 mg, 0.124 mmol) to provide A-006 (4.2 mg, 0.006 mmol, 8% overall yield). (MW_{Calc}+H=723.38; MW_{Obs}=723.60).

A-007 was prepared in a similar fashion to A-001 starting with compound 18 (50.0 mg, 0.075 mmol) and commercially available tert-butyl 4-oxa-1,9-diazaspiro[5.5]undecane-9-carboxylate (31.8 mg, 0.124 mmol) to provide A-007 (8.2 mg, 0.011 mmol, 15% overall yield). (MW_{Calc}+H=723.38; MW_{Obs}=723.60).

A-008 was prepared in a similar fashion to A-001 starting with compound 18 (50.0 mg, 0.075 mmol) and commercially available tert-butyl 8-oxa-2,5-diazaspiro[3.5]nonane-2-carboxylate (28.3 mg, 0.124 mmol) to provide A-008 (28.3 mg, 0.040 mmol, 53% overall yield) (MW_{Calc}+H=695.35; MW_{Obs}=695.60).

A-009 was prepared in a similar fashion to A-001 starting with compound 18 (10.0 mg, 0.015 mmol) and commercially available tert-butyl methyl(morpholin-3-ylmethyl)carbamate (5.2 mg, 0.0.23 mmol) to provide A-009 (0.4 mg, 0.005 mmol, 4% overall yield) (MW_{Calc}+H=697.4; MW_{Obs}=697.7).

A-010 and A-011 were prepared in a similar fashion to A-001 starting with compound 18 (335 mg, 0.504 mmol) and commercially available tert-butyl 9-methyl-2,6,9-triazaspiro[4.5]decane-2-carboxylate (193 mg, 0.756 mmol) to provide A-010 (60 mg, 0.083 mmol, 27% overall yield) (MW_{Calc}+H=722.39; MW_{Obs}=722.51) and A-011 (65.2 mg, 0.092 mmol, 30% overall yield) (MW_{Calc}+H=722.39; MW_{Obs}=722.23) after purification using a chiral reverse-phase HPLC column, collection of the desired fractions, and concentration to dryness under vacuum. (330 mg, 0.365 mmol, 72.4% of protected intermediate as a mixture of diastereomers) (MW_{Calc}+H=722.39; MW_{Obs}=722.23).

A-012 and A-013 were prepared in a similar fashion to A-001 starting with compound 18 (230 mg, 0.346 mmol) and commercially available 9-((9H-fluoren-9-yl)methyl) 2-(tert-butyl) 2,6,9-triazaspiro[4.5]decane-2,9-dicarboxylate (241 mg, 0.519 mmol) to provide after purification A-012 (24.9 mg, 0.035 mmol, 62% overall yield) (MW_{Calc}+H=708.38; MW_{Obs}=708.5) and A-013 (25 mg, 0.036 mmol, 54% overall yield) (MW_{Calc}+H=708.38; MW_{Obs}=708.5).

A-014 and A-015 were prepared in a similar fashion to A-001 starting with compound 18 (60 mg, 0.0.90 mmol) and commercially available tert-butyl 9-acetyl-2,6,9-triazaspiro[4.5]decane-2-carboxylate (38 mg, 0.135 mmol) to provide A-014 (60 mg, 0.083 mmol, 27% overall yield) (FW=721.85; MW_{Calc}+H=722.39; MW_{Obs}=722.51] and a mixture of A-014 (6.57 mg, 0.0087 mmol, 32.7% overall yield) (MW_{Calc}+H=750.39; MW_{Obs}=750.5) and A-015 (7.19 mg, 0.0095 mmol, 35.5%) (MW_{Calc}+H=723.39; MW_{Obs}=723.4) after purification using a chiral reverse-phase HPLC column, collection of the desired fractions, and concentration to dryness under vacuum. DL 2804.095.

A-016 was prepared in a similar fashion to A-001 starting with compound 18 (20.0 mg, 0.03 mmol) and commercially available tert-butyl 9-methyl-8-oxo-2,6,9-triazaspiro[4.5]decane-2-carboxylate (10 mg, 0.036 mmol) to provide A-016 (4.7 mg, 0.006 mmol, 21% overall yield) (MW_{Calc}+H=736.37; MW_{Obs}=736.5).

A-017 was prepared in a similar fashion to A-001 starting with compound 18 (24.0 mg, 0.036 mmol) and commercially available tert-butyl 1,6-diazaspiro[3.4]octane-1-carboxylate (11.5 mg, 0.054 mmol) to provide A-017 (10.4 mg, 0.027 mmol, 42% overall yield) (MW_{Calc}+H=679.35; MW_{Obs}=679.35). Compounds have been purified by water/acetonitrile gradient containing 0.1% formic acid on a reversed-phase C18 Xselect.

A-018 was prepared in a similar fashion to A-001 starting with compound 18 (25.0 mg, 0.038 mmol) and commercially available tert-butyl 1,6-diazaspiro[3.4]octane-6-carboxylate (20 mg, 0.094 mmol) to provide A-018 (17.8 mg, 0.026 mmol, 69% overall yield) 0(MW_{Calc}+H=679.35; MW_{Obs}=679.39).

A-019 and A-020 were prepared in a similar fashion to A-001 starting with compound 18 (25.0 mg, 0.038 mmol) and commercially available tert-butyl 1,7-diazaspiro[4.4]nonane-7-carboxylate (21.3 mg, 0.094 mmol) to provide A-019 (9.7 mg, 0.014 mmol, 37% overall yield), and A-020 (9.7 mg, 0.014 mmol, 37% overall yield) after purification using a chiral reverse-phase HPLC column, collection of the desired fractions, and concentration to dryness under vacuum. (MW_{Calc}+H=693.37; MW_{Obs}=693.39).

A-021 was prepared in a similar fashion to A-001 starting with compound 18 (28.0 mg, 0.042 mmol) and commercially available tert-butyl 1,6-diazaspiro[3.3]heptane-6-carboxylate (21 mg, 0.105 mmol) to provide A-021 (16.5 mg, 0.0248 mmol, 59% overall yield) (MW_{Calc}+H=665.34; MW_{Obs}=665.33).

A-022 was prepared in a similar fashion to A-001 starting with compound 18 (28.0 mg, 0.042 mmol) and commercially available tert-butyl 2,6-diazaspiro[4.5]decane-2-carboxylate (25.3 mg, 0.105 mmol) to provide A-022 (4.2 mg, 0.0059 mmol, 14% overall yield) (MW_{Calc}+H=707.38; MW_{Obs}=707.6).

A-023 was prepared in a similar fashion to A-001 starting with compound 18 (30.0 mg, 0.042 mmol) and commercially available tert-butyl 1,7-diazaspiro[3.5]nonane-7-carboxylate (25.3 mg, 0.105 mmol) to provide A-023 (21.9 mg, 0.032 mmol, 70% overall yield) (MW_{Calc}+H=693.37; MW_{Obs}=693.36).

A-024 was prepared in a similar fashion to A-001 starting with compound 18 (20.0 mg, 0.03 mmol) and commercially available 3-(pyridin-3-yl)morpholine (7.4 mg, 0.045 mmol) to provide A-024 (11 mg, 0.017 mmol, 58% overall yield) (MW_{Calc}+H=631.29; MW_{Obs}=631.37).

A-025 was prepared in a similar fashion to A-001 starting with compound 18 (44.1 mg, 0.066 mmol) and commercially available tert-butyl 2-methyl-1,7-diazaspiro[4.4]nonane-7-carboxylate (24 mg, 0.10 mmol) to provide A-025 (14.1 mg, 0.02 mmol, 56% overall yield) (MW_{Calc}+H=707.38; MW_{Obs}=707.38).

A-026 was prepared in a similar fashion to A-001 starting with compound 18 (30 mg, 0.045 mmol) and commercially available tert-butyl 3-amino-3-methyl-pyrrolidine-1-carboxylate (13.6 mg, 0.10 mmol) to provide A-026 (13.9 mg, 0.020 mmol, 47% overall yield) (MW_{Calc}+H=667.35; MW_{Obs}=667.37).

### Preparation of A-027

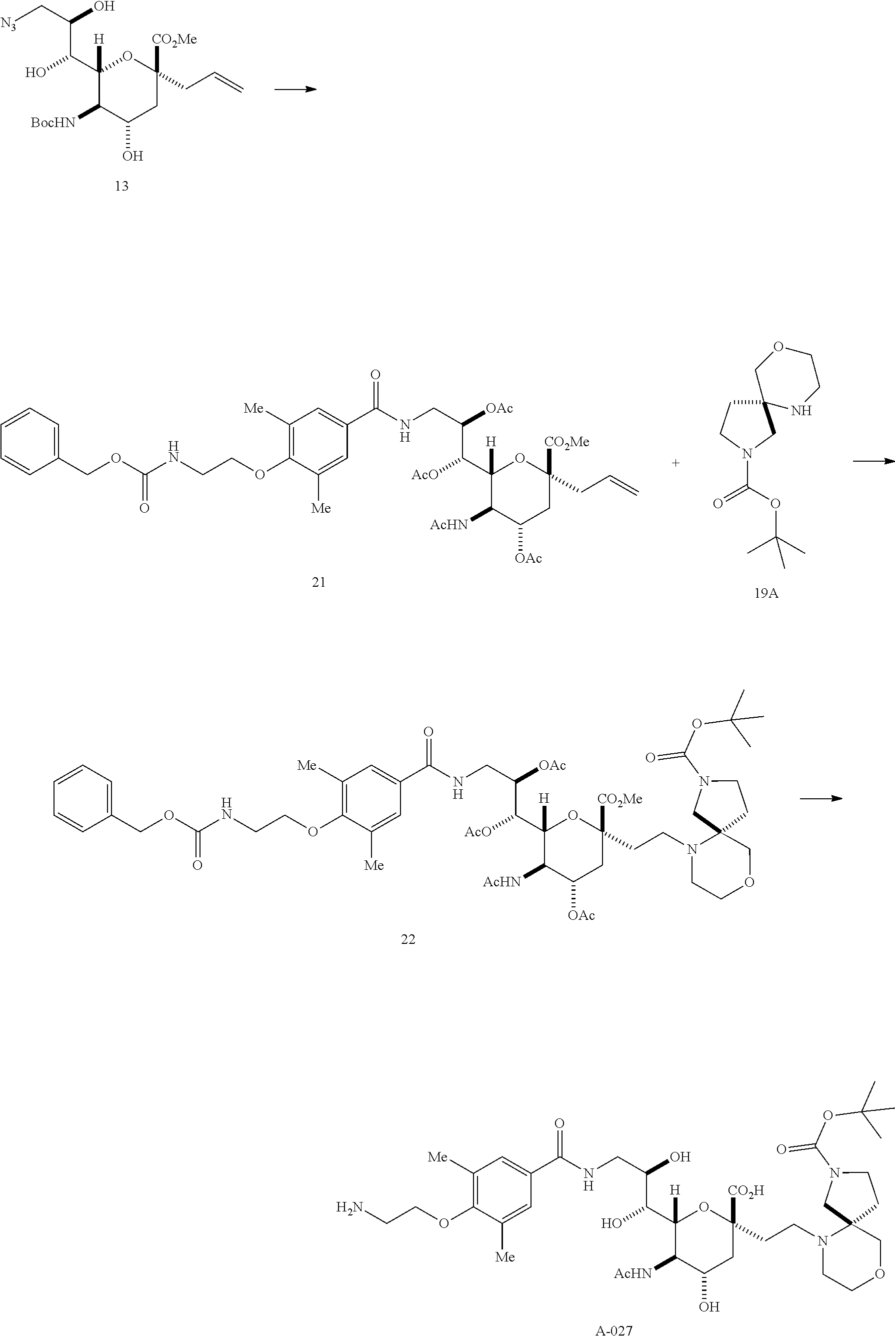

To a stirred solution of methyl (2R,4S,5R,6R)-2-allyl-6-((R,2R)-3-azido-1,2-dihydroxypropyl)-5-((tert-butoxycarbonyl)amino)-4-hydroxytetrahydro-2H-pyran-2-carboxylate (13, 75 mg, 0.174 mmol) in THF (3 mL) and water (0.5 mL) at room temperature was added trimethylphosphine (0.523 mL, 0.523 mmol) followed by stirring for 16 h. The completed intermediate reaction was concentrated and azeotroped to dryness with toluene (3×10 mL ea). The crude intermediate was used directly in the next reaction.

To a stirred solution of the crude amine from above in acetonitrile (1 mL) at room temperature was added commercially available 4-(2-(((benzyloxy)carbonyl)amino)ethoxy)-3,5-dimethylbenzoic acid (0.075 g, 0.218 mmol) and HOBt (0.013 g, 0.087 mmol) followed by triethylamine (0.061 ml, 0.436 mmol) and EDC (0.045 g, 0.235 mmol). The reaction mixture was stirred for 16 h, after which time water (5 mL) and EtOAc (20 mL) were added. The resultant layers were separated, the organic layer was washed with brine (5 mL), and the EtOAc layer dried over Na₂SO₄, filtered, and evaporated to dryness. The crude intermediate was used directly in the next reaction.

To a stirred solution of the crude intermediate in DCM (5 mL) at room temperature was added TFA (0.1 mL, 1.31 mmol). The reaction was stirred for 15 min, after which time it was concentrated and azeotroped to dry with toluene (3×5 mL ea). The crude intermediate was used directly in the next reaction.

To a stirred solution of the final crude intermediate in pyridine (0.5 mL) at room temperature was added acetic anhydride (0.3 mL, 3.17 mmol) followed by stirring for 24 h. The completed reaction was diluted with EtOAc (20 mL), washed with 1N HCl (5 mL), sat. NH₄Cl (5 mL), and brine (5 mL). The organic layer was dried over Na₂SO₄, filtered, and evaporated to dryness to provide compound 21 (75 mg, 0.094 mmol, 54%) as a pure product. (MW_{Calc}+H=798.86; MW_{Obs}=799.45).

To a stirred solution of (1R,2R)-1-((2R,3R,4S,6R)-3-acetamido-4-acetoxy-6-allyl-6-(methoxycarbonyl)tetrahydro-2H-pyran-2-yl)-3-(4-(2-(((benzyloxy)carbonyl)amino)ethoxy)-3,5-dimethylbenzamido)propane-1,2-diyl diacetate (21, 160 mg, 0.201 mmol) dioxane (3 mL) and water (1 mL) at room temperature was added 2,6-lutidine (46.7 μl, 0.401 mmol) and osmium tetroxide (79 μl, 0.010 mmol), followed by sodium periodate (172 mg, 0.802 mmol). The reaction mixture was stirred 3 h, after which time it was diluted with water (5 mL) and extracted with DCM (3×10 mL ea). The combined organic layers were washed with water (5×5 mL ea), dried over MgSO₄, filtered and concentrated to dryness to provide a crude black oil, which was used directly in the next reaction.

To a stirred solution of the crude aldehyde from above in 1,2 dichloroethane (3 mL) at room temperature was added tert-butyl (S)-9-oxa-2,6-diazaspiro[4.5]decane-2-carboxylate (19A, 60.7 mg, 0.251 mmol), acetic acid (86 μl, 1.50 mmol) and dry 4A MS (220 mg). The mixture was stirred for 3 h, after which time sodium triacetoxyborohydride (85 mg, 0.401 mmol) was added followed by stirring for an additional 3 h. The completed reaction was diluted with NaHCO₃ (3 mL) and extracted with EtOAc (3×5 mL ea). The combined organic layers were washed with brine (3 mL), and concentrated to dryness to provide crude compound 22, which was used directly in the next reaction.

To a stirred solution of the crude 22 in 1:1 EtOAc:EtOH (10 mL) at room temperature was added 10% Pd/C (50 mg) followed by placing under H₂ atmosphere (balloon pressure) for 6 h. The completed reaction was filtered over a pad of Celite (5 g), eluting with EtOAc (2×5 mL ea), and the resultant filtrate was concentrated, and azeotroped to dry with MeOH (2×5 mL). The crude residue was dissolved in MeOH (0.5 mL) followed by 1 N aq. NaOH (0.7 mL, 0.070 mmol) and stirred for 36 h. The final reaction mixture was neutralized with 1 N HCl (0.7 mL, 0.07 mmol) and directly purified over an HPLC column to provide compound A-027 (2.9 mg, 0.004 mmol, 2% overall yield from 21) after combining the desired fractions, concentration and drying under high vacuum. (MW_{Calc}+H=752.88; MW_{Obs}=752.60).

### Preparation of A-028 and A-029

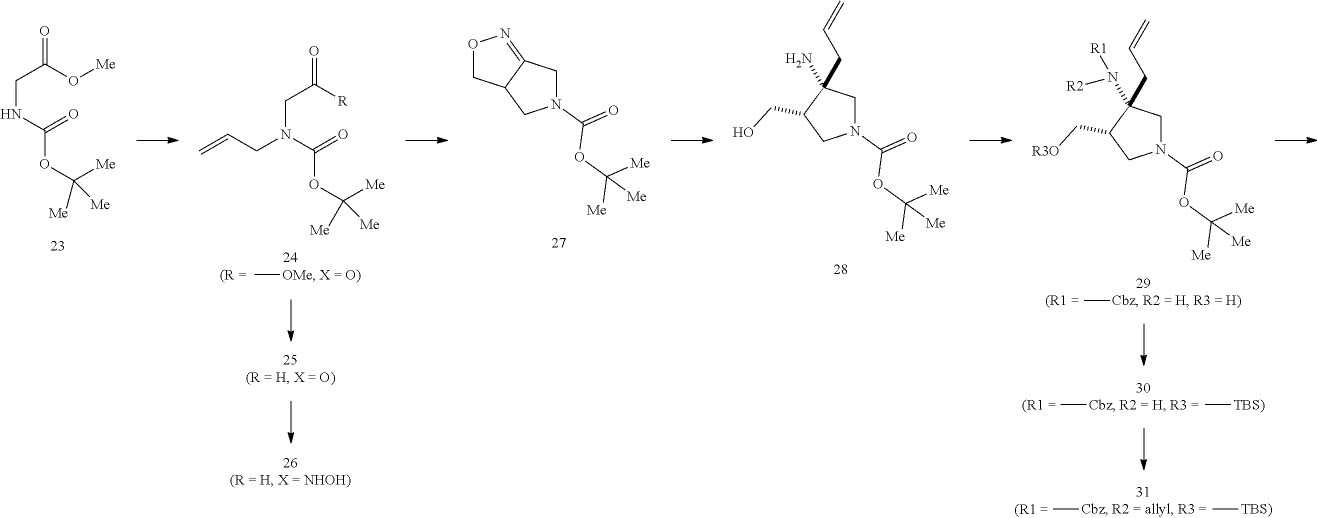

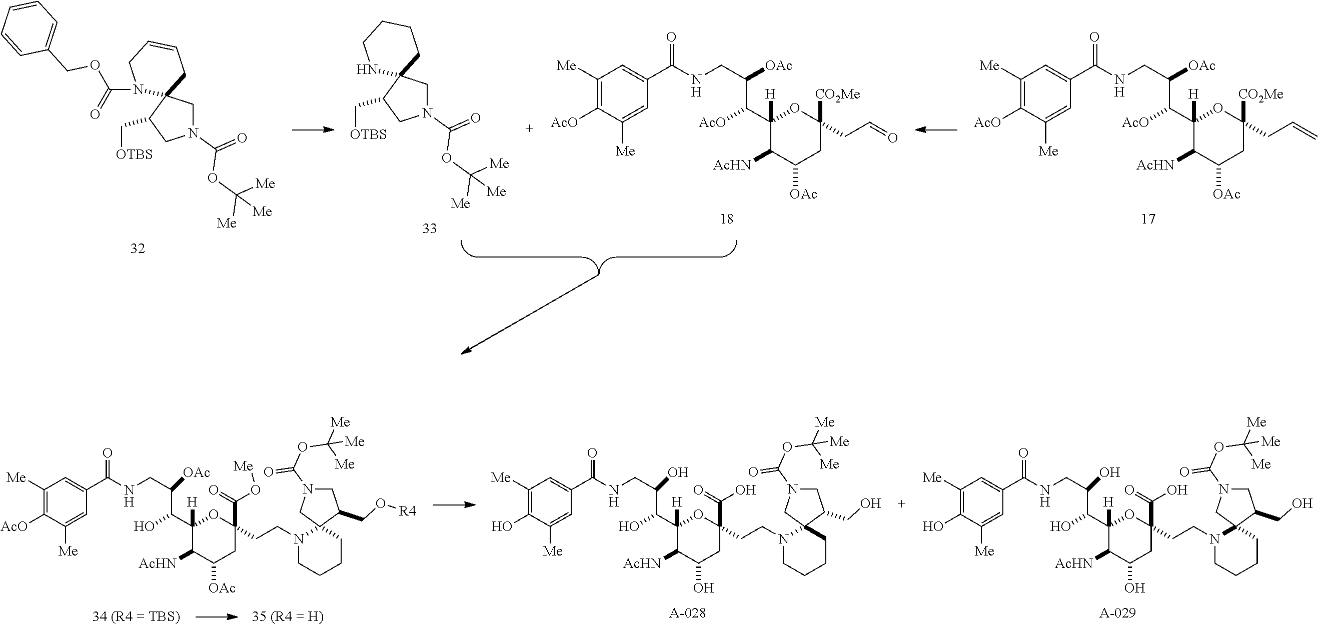

To a stirred solution of methyl (tert-butoxycarbonyl)glycinate (23, 20 g, 105.7 mmol) and allyl bromide (13.8 ml, 159.5 mmol) in DMF (207 mL) cooled to −15° C. was added 60% sodium hydride (6.34 g, 158.5 mmol) slowly portion wise maintaining the temperature below −5° C. The reaction was stirred at −5° C. for 5 h after which time it was cautiously quenched with sat. NH₄Cl (100 mL) and water (100 mL) followed by extracting with ethyl acetate (2×500 mL ea), washed with half sat. brine (100 mL), dried over Na₂SO₄, filtered, concentrated, and azeotroped to dry with toluene (2×50 mL ea) to provide compound 24 (22.61 g, 98.62 mmol, 93%).

To a stirred solution of methyl N-allyl-N-(tert-butoxycarbonyl)glycinate (24, 22.61 g, 98.62 mmol) in DCM (327 mL) was added dropwise 1 M DIBAL-H (123 ml, 123.27 mmol) in DCM at −78° C. over a 20 min period. The reaction was stirred for 3 h at −78° C. after which time it was quenched with a slow dropwise addition of methanol (4.99 ml, 123.27 mmol) followed by the addition of 0.8 M NaOH (765 ml, 612.00 mmol) and DCM (180 mL). The quenched reaction was allowed to warm to room temperature after which time the layers were separated, the aqueous layer was extracted with DCM (100 mL). The combined organic layers were with water (100 mL), 1:1 water:brine (50 mL), dried over Na₂SO₄, filtered, concentrated, and azeotroped to dry with MeCN (50 mL) to provide compound 25 (15.92 μm, 79.90 mmol, 81%) as a crude oil.

To a stirred solution of tert-butyl allyl(2-oxoethyl)carbamate (25, 15.92 g, 79.90 mmol) in DCM (120 mL) and methanol (60 ml, 1483.053 mmol) was added hydroxylamine hydrochloride (15.77 g, 226.92 mmol) and sodium acetate (18.61 g, 226.92 mmol) at room temperature followed by stirring for 72 h. The completed reaction was poured into water (300 mL), the layers separated, and the aqueous layer was extracted with DCM (150 mL). The combined organic layers were washed with 1:1 water:brine (50 mL), dried over Na₂SO₄, filtered, and concentrated to dryness to provide compound 26 (15.46 g, 72.2 mmol 90%) as a crude oil.

To a stirred solution of tert-butyl allyl(2-(hydroxyimino)ethyl)carbamate (26, 15.46 g, 72.15 mmol) in DCM (150 mL) at room temperature was added 5% aq. sodium hypochlorite (170 mL, 137.71 mmol) dropwise over 60 min. The reaction was stirred at room temperature for 1 h after which time the layers were separated, the aqueous layer was extracted with DCM (200 mL), and the combined organic layers were washed with 1:1 water:brine (2×50 mL ea), dried over Na₂SO₄, filtered, and concentrated.

The crude product was purified over a Biotage SNAP column (100 g) eluting with 2 CV heptane; 2 CV, 0% to 5% EtOAc in heptane; 2 CV, 5% EtOAc in heptane; 6 CV, 5% to 30% EtOAc in heptane; 2 CV, 30% to 50% EtOAc in heptane; 2 CV, 50% EtOAc in heptane; 2 CV, 50% to 80% EtOAc in heptane; and 2 CV, 80% to 100% EtOAc in heptane to provide after combining and concentration of the desired fractions to dryness compound 27 (9.49 g, 44.7 mmol, 62%). (MW_{Calc}+Na=235.12; MW_{Obs}=235.17).

To a stirred solution of tert-butyl 3a,4-dihydro-3H-pyrrolo[3,4-c]isoxazole-5(6H)-carboxylate (27, 2.2 g, 10.365 mmol) in THF (15 mL) and toluene (15 mL) at −78° C. under a N2 atmosphere, was added boron trifluoride etherate (1.5 mL, 11.837 mmol) followed by the dropwise allylmagnesium bromide (12 mL, 12.00 mmol) in THF maintaining the temperature below −60° C. The reaction mixture was stirred at less than −70° C. for 2.5 h after which time it was carefully quenched with sat. aqueous NH₄Cl (10 mL), and the mixture was allowed to warm to room temperature. The completed reaction was treated with sat. NaHCO₃ until reached pH 7-8, followed by extraction with ethyl acetate (2×75 mL ea). The combined organic layers were washed with 1:1 water:brine (50 mL), dried over Na₂SO₄, filtered, and concentrated to dryness to provide the crude intermediate (2.99 g).

The crude intermediate was redissolved in THF (40 mL) and acetic acid (5 mL) and cooled to 0° C. after which time zinc (2.50 g, 38.24 mmol) was added, and stirred for 10 min. The reaction was warmed to room temperature and stirred for 24 h after which time Celite (11 g) was added, and then filtered over a plug of Celite (11 g) Celite plug. The filter pad was washed with ethyl acetate (350 mL), and then sat. sodium bicarbonate (80 g) was added to the filtrate followed by stirring for 15 min. The layers were separated, and the aqueous layer was extracted with ethyl acetate (100 mL). The combined organic layers were washed with 1:1 water:brine (50 mL), dried over Na₂SO₄, filtered, and concentrated to dryness to provide compound 28 (2.67 g, 10.36 mmol, 100%) as a crude syrup as a racemic mixture. (MW_{Calc}+H+=257.19; MW_{Obs}=256.90).

To a stirred solution of crude tert-butyl (3S,4S)-3-allyl-3-amino-4-(hydroxymethyl)pyrrolidine-1-carboxylate (28, 500. mg, 1.95 mmol) in DCM (10 mL) at 0° C. was added sat. aq. sodium bicarbonate (15 mL) followed by 3 M benzyl chloroformate (0.715 ml, 2.15 mmol) in toluene kept stirring. The reaction mixture was stirred at 0° C. for 2 h after which time an additional amount of 3 M benzyl chloroformate (0.130 mL, 0.39 mmol) in toluene was added and stirred for 1.5 h. The completed reaction was quenched with isopropylamine (0.2 mL, 2.335 mmol), stirred for 15 min at 0° C., and then extracted with ethyl acetate (2×20 mL each). The combined organic layers were washed with 1:1 water:brine (20 mL), dried over Na₂SO₄, filtered, and concentrated to dryness. Purification over a Biotage SNAP column (25 g) eluting with eluting with 0-100% ethyl acetate in heptane (10 CV) provided compound 29 (340.0 mg, 0.87 mmol, 45% from compound 26) after collection of the desired fractions, concentration and vacuum to dryness. (MW_{Calc}+Na⁺=413.22; MW_{Obs}=413.12).

To a stirred solution of tert-butyl (3S,4S)-3-allyl-3-(((benzyloxy)carbonyl)amino)-4-(hydroxymethyl)pyrrolidine-1-carboxylate (29, 340 mg, 0.87 mmol) and imidazole (237 mg, 3.48 mmol) in DMF (3.4 mL) was added tert-butyldimethylsilyl chloride (262 mg, 1.74 mmol) at room temperature. The reaction was stirred for 23 h after which time it was diluted with, kept stirring. Upon completion by TLC the reaction was worked up with 1:1 water:MTBE (30 mL) and the layers separated. The aqueous layer was extracted with MTBE (10 mL), and the combined organic layers were washed with sat. NaHCO₃ (10 mL), 1:1 water:brine (20 mL), dried over Na₂SO₄, filtered, and concentrated to dryness. Purification over a Biotage SNAP column (25 g) eluting with 0-40% ethyl acetate in heptane (10 CV) provided compound 30 (367 mg, 0.728 mmol, 84%) after collection of the desired fractions, concentration and vacuum to dryness. (MW_{Calc}+Na=527.30; MW_{Obs}=527.30).

To a stirred solution of tert-butyl (3S,4S)-3-allyl-3-(((benzyloxy)carbonyl)amino)-4-(((tert-butyldimethylsilyl)oxy)methyl)pyrrolidine-1-carboxylate (30, 367 mg, 0.728 mmol) and allyl bromide (189 μL, 2.184 mmol) in DMF (3.0 mL) cooled at 0° C. was slowly added NaH (87 mg, 2.184 mmol). The reaction was stirred for 2 h after which time it was slowly quenched with a mixture of sat. NH4C1 (1.5 mL) and water (1.5 mL). The mixture was extracted with MTBE (70 mL), and the organic layer was washed with 1:1 water:brine (20 mL), dried over Na₂SO₄, filtered, and concentrated to dryness. Purification over a Biotage SNAP column (25 g) eluting with 0-40% ethyl acetate in heptane (10 CV) provided compound 31 (397 mg, 0.728 mmol, 100%) after collection of the desired fractions, concentration and vacuum to dryness. (MW_{Calc}+Na=567.33; MW_{Obs}=567.28).

To a stirred solution of tert-butyl (3S,4S)-3-allyl-3-(allyl((benzyloxy)carbonyl)amino)-4-(((tert-butyldimethylsilyl)oxy)methyl)pyrrolidine-1-carboxylate (31, 397 mg, 0.728 mmol) in toluene (20 ml, 187.756 mmol) was added Hoveyda-Grubbs Catalyst 2nd Generation (46.2 mg, 0.073 mmol). The reaction mixture was degassed and refilled with N₂ three times, and then stirred with 80° C. for 8 h. The completed reaction was cooled to room temperature, concentrated, and loaded onto a Biotage SNAP column (25 g) eluting with 0-40% ethyl acetate in heptane (10 CV) provided compound 32 (360 mg, 0.697 mmol, 95%) after collection of the desired fractions, concentration and vacuum to dryness. (MW_{Calc}+Na=539.30; MW_{Obs}=539.24).

To a stirred solution of 6-benzyl 2-(tert-butyl) (4S,5S)-4-(((tert-butyldimethylsilyl)oxy)methyl)-2,6-diazaspiro[4.5]dec-8-ene-2,6-dicarboxylate (32, 0.36 g, 0.697 mmol) in ethyl acetate (7.2 mL) was added acetic acid (0.040 mL, 0.697 mmol) followed by 5% Pd—C(0.089 g, 0.042 mmol). The reaction mixture was stirred under a low-pressure hydrogen atmosphere for 16 h after which time it was degassed and purged with N₂ gas 3 times followed by filtering over Celite (2 g), and eluted with MeOH (10 mL). The filtrate was concentrated to dryness and then diluted with 1:1 ethyl acetate: sat. NaHCO₃ (40 mL). The layers were separated, and the organic layer was washed with water:brine (20 mL), dried over Na₂SO₄, filtered, and concentrated to dryness. Purification over a Biotage SNAP column (25 g) eluting with 0-100% ethyl acetate in heptane (10 CV) provided compound 33 (207 mg, 0.538 mmol, 77%) after collection of the desired fractions, concentration and vacuum to dryness. (MW_{Calc}+Na=407.28; MW_{Obs}=407.33.

To a stirred solution of (1R,2R)-1-((2R,3R,4S,6R)-3-acetamido-4-acetoxy-6-allyl-6-(methoxycarbonyl)tetrahydro-2H-pyran-2-yl)-3-(4-acetoxy-3,5-dimethylbenzamido)propane-1,2-diyl diacetate (17, 51 mg, 0.077 mmol) in methanol (1.0 mL) was subjected to ozonolysis at −78° C. for 15 min after which time it was quenched with dimethylsulfide (0.5 mL). The completed reaction was allowed to warm to room temperature followed by concentration to dryness to provide crude compound 18.

Crude compound 18 and tert-butyl (4S,5S)-4-(((tert-butyldimethylsilyl)oxy)-methyl)-2,6-diazaspiro[4.5]decane-2-carboxylate (33, 35.4 mg, 0.092 mmol) were azeotroped to dry with acetonitrile (3×10 mL) followed by dissolving in dichloroethane (1.5 mL). To the solution was added acetic acid (26.4 μL, 0.462 mmol) and then 4A MS (0.5 g) followed by stirring for 2.5 h. Sodium triacetoxyborohydride (82 mg, 0.385 mmol) was added, and the reaction was stirred at room temperature for 72 h. The completed reaction was diluted with 1:1 ethyl acetate in sat. NaHCO₃ (10 mL), stirred, and the layers separated. The aqueous layer was extracted with EA (5 mL), and the combined organic layer was washed with 1:1 water:brine (5 mL), dried over Na₂SO₄, filtered, and concentrated to dryness to provide crude compound 34 (77 mg, 0.075 mmol, 97%). (MW_{Calc}+H=1033.53; MW_{Obs}=1033.49).

To a stirred solution of crude (1R,2R)-1-((2R,3R,4S,6R)-3-acetamido-4-acetoxy-6-(2-((4S,5S)-2-(tert-butoxycarbonyl)-4-(((tert-butyldimethylsilyl)oxy)methyl)-2,6-diazaspiro[4.5]decan-6-yl)ethyl)-6-(methoxycarbonyl)tetrahydro-2H-pyran-2-yl)-3-(4-acetoxy-3,5-dimethylbenzamido)propane-1,2-diyl diacetate (34, 77.2 mg, 0.075 mmol) in THF (4.0 mL) at 0° C. was added 1 M TBAF (250 μL, 0.25 mmol) in THF. The reaction mixture was stirred at 0° C. for 3 h after which time 1 M TBAF (100 μL, 0.10 mmol) in THF was added, and the reaction was stirred at 0° C. for 15 h. The completed reaction was diluted with 1:1 ethyl acetate in sat. NaHCO₃ (10 mL), stirred, and the layers separated. The aqueous layer was extracted with EA (5 mL), and the combined organic layer was washed with 1:1 water:brine (5 mL), dried over Na₂SO₄, filtered, and concentrated to dryness. Purification over a Biotage SNAP column (25 g) eluting with 0-40% ethyl acetate in heptane (10 CV) provided compound 35 (28.1 mg, 0.031 mmol, 41%) as a diastereomixture of two compounds after collection of the desired fractions, concentration and vacuum to dryness. (MW_{Calc}+H=919.45; MW_{Obs}=919.45).

To a stirred solution of (1R,2R)-1-((2R,3R,4S,6R)-3-acetamido-4-acetoxy-6-(2-((4S,5S)-2-(tert-butoxycarbonyl)-4-(hydroxymethyl)-2,6-diazaspiro[4.5]decan-6-yl)ethyl)-6-(methoxycarbonyl)-tetrahydro-2H-pyran-2-yl)-3-(4-acetoxy-3,5-dimethylbenzamido)propane-1,2-diyl diacetate (35, 6 mg, 6.529 μmol) (dr 1:1) in MeOH (0.3 mL) and water (50 μL) was added 1 N NaOH (102 μl, 0.102 mmol) at room temperature followed by stirring for 14 h. 2 N NaOH (46 μL, 0.132 mmol) was added and the reaction was stirred for an additional 24 h. The reaction was quenched with 1 N HCl (230 μl, 0.23 mmol) to pH ca. 6-7 and directly purified over a chiral HPLC column to provide A-028 (3.2 mg, 0.004 mmol, 64%) (MW_{Calc}+H=736.39; MW_{Obs}=736.33) and A-029 (2.2 mg, 0.003 mmol, 44%) (MW_{Calc}+H=736.39; MW_{Obs}=736.31).

### Preparation of A-030 to A-035

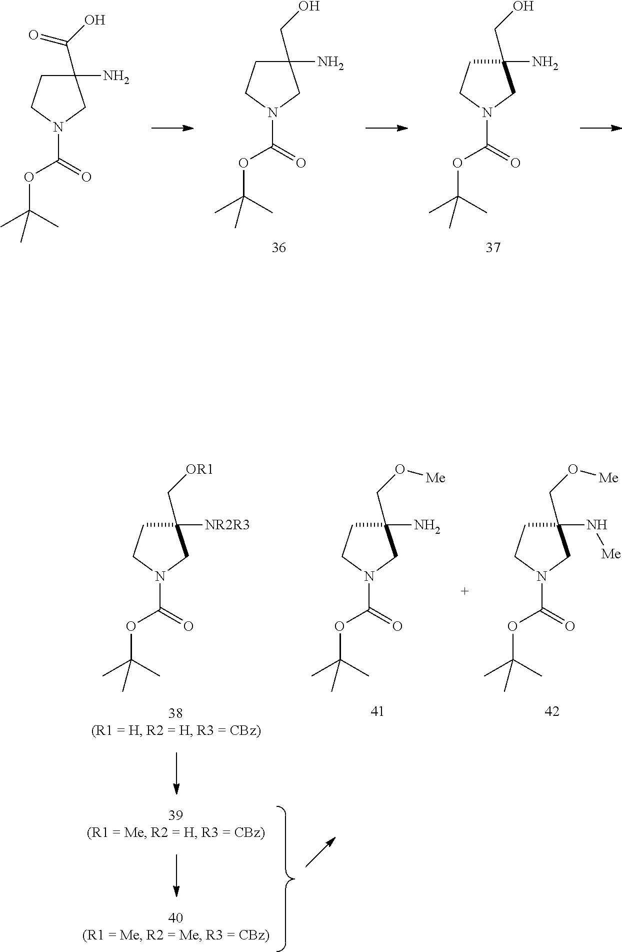

To a stirred solution of sodium borohydride (0.789 g, 20.85 mmol) in THF (50.0 mL) under a N₂ atmosphere was added commercially available 3-amino-1-(tert-butoxycarbonyl)pyrrolidine-3-carboxylic acid (2.0 g, 8.686 mmol). The reaction was cooled to 0° C. followed by the dropwise addition of iodine (2.21 g, 8.69 mmol) in THF (14.00 mL). After gas evolution was completed, the reaction was heated to reflux and stirred for 16 h. The completed reaction was cooled to room temperature, and was quenched by the slow addition of MeOH (7.03 mL) followed by concentration. The residue was dissolved in 20% aqueous KOH (122 mL, 434.29 mmol) and stirred at room temperature for 4 hours followed by extraction with DCM (3×100 mL ea). The combined organic layers were dried over Na₂SO₄, filtered, and concentrated to obtain crude compound 36 (1.85 g, 8.55 mmol, 98%), which was used in the next reaction without further purification.

To a stirred solution of tert-butyl 3-amino-3-(hydroxymethyl)pyrrolidine-1-carboxylate (36, 0.30 g, 1.39 mmol) in 2-propanol (2.4 mL) was added (2S,3S)-2,3-bis((4-methylbenzoyl)oxy)succinic acid (0.268 g, 0.694 mmol) followed by stirring at room temperature until mostly dissolved. The resultant solution was heated at 65° C. for 1 h after which time the resulting white mixture was cooled to room temperature. The white solid suspension was filtered, the filter pad washed with cold 2-propanol (1 mL), and the filter cake was dried under vacuum at 45° C. for 16 h to provide crude tert-butyl (S)-3-amino-3-(hydroxymethyl)pyrrolidine-1-carboxylate (2S,3S)-2,3-bis((4-methylbenzoyl)oxy)-succinate (247 mg, 0.603 mmol, 43%).

Tert-butyl (S)-3-amino-3-(hydroxymethyl)pyrrolidine-1-carboxylate (2S,3S)-2,3-bis((4-methylbenzoyl)oxy)succinate (209 mg, 0.51 mmol) was suspended in water (1.045 mL) and EtOAc (1.045 mL) and cooled to 0-5° C. with stirring after which time 6 M HCl (85 μL, 0.51 mmol) was added dropwise followed by stirring at 0-5° C. for 1 h. The layers were separated, and the aqueous layer was extracted with EtOAc (1 mL). The aqueous layer was cooled to 0-5° C., and then treated with 3 M NaOH (170 μl, 0.51 mmol) followed by stirring for 1 h. The resultant aqueous solution was lyophilized to a dry powder, which was then suspended in EtOH (4 mL) and stirred for 4 hours at room temperature. The white suspension was filtered through a pad of Celite, washed with EtOH (2 mL), and the filtrate was concentrated and dried under vacuum to provide compound 37 (98.9 mg, 0.453 mmol, 89%).

To a stirred solution of tert-butyl 3-amino-3-(hydroxymethyl)pyrrolidine-1-carboxylate (37, 1.50 g, 6.94 mmol) in THF (40 mL) and aqueous sodium carbonate (0.956 g, 9.02 mmol, 40 mL) at 0° C. was added dropwise benzyl carbonochloridate (4.48 mL, 8.32 mmol). The reaction mixture was stirred at 0° C. for 12 h after which time the completed reaction was extracted with EtOAc (2×50 mL ea). The organic layer was washed with aqueous sodium carbonate (20 mL), dried over potassium carbonate, filtered, and concentrated to dryness to provide compound 38 (2.0 g, 5.71 mmol, 82%). (MW_{Calc}+H=315.19; MW_{Obs}=351.05).

To a stirred slurry/solution of tert-butyl (S)-3-(((benzyloxy)carbonyl)amino)-3-(hydroxymethyl)pyrrolidine-1-carboxylate (38, 0.50 g, 1.427 mmol) in DCM (15 mL) at 5° C. was added methyl iodide (0.184 mL, 0.686 mmol), tetrabutylammonium hydrogen sulfate (73 mg, 0.214 mmol) and 50% aq NaOH (0.90 mL). The reaction mixture was stirred vigorously for 4 h maintaining the temperature between 5-15° C. after which time the completed the reaction was diluted DCM (15 mL) and stirred at room temperature for 4 h. The resultant mixture was acidified to pH 6 with the addition of 1N HCl, the layers were separated, and the aqueous layer was extracted with DCM (2×10 mL ea). The combined organic layers were dried with MgSO₄, filtered, concentration, the crude residue was purified over a Biotage SNAP column (25 g) eluting with 10-100% ethyl acetate in heptane (10 CV) to provide compound 39 (0.25 g, 0.686 mmol, 48%) after collection of the desired fractions, concentration and vacuum to dryness. (MW_{Calc}+H=365.20; MW_{Obs}=365.14).

To a stirred solution of tert-butyl (S)-3-(((benzyloxy)carbonyl)amino)-3-(methoxymethyl)pyrrolidine-1-carboxylate (39, 100 mg, 0.274 mmol) in THF (2 mL) was added 60% sodium hydride (16.5 mg, 0.412 mmol) at room temperature, and stirred for 30 min. Imidazole (0.2 mg, 0.003 mmol) was added followed by stirring for 20 min. after which time iodomethane (22 uL, 0.357 mmol) was added, and reaction mixture was warmed to 45° C. The final mixture was stirred for 3 h at 45° C. after which time it was cooled to room temperature, and quenched with sat. aq ammonium chloride solution. The resultant mixture was extracted with ethyl acetate (3×2 mL ea), and the combined organic layers were dried over Na₂SO₄, filtered, and concentrated. The crude product was filtered over silica gel pad (5 g) eluting with ethyl acetate (20 mL) and the filtrate was concentrated to provide a mixture of compounds 39 and 40 (90 mg, ˜0.24 mmol, ˜87%) in a 2 to 7 ratio via HPLC, which were used in the next step as a mixture.

To a degassed and stirred solution of tert-butyl (S)-3-(((benzyloxy)carbonyl)amino)-3-(methoxymethyl)pyrrolidine-1-carboxylate and tert-butyl (S)-3-(((benzyloxy)carbonyl)-(methyl)amino)-3-(methoxymethyl)pyrrolidine-1-carboxylate (39 and 40, 90 mg, ˜0.24 mmol) in MeOH (5 mL) was added 10% Pd/C (10 mg), placed hydrogen atmosphere and stirred for 14 h at room temperature. The completed reaction was filtered over Celite (5 g), eluted with methanol (10 mL), and concentrated. The crude product was passed through a pad of silica gel (5 g) eluted with ethyl acetate (20 mL) to give a mixture of compounds 41 and 42, after concentration to dry. The mixture was used in the next reaction without further purification.

A-030 and A-031 were prepared in a similar fashion to A-001 starting with compound 18 (30 mg, 0.045 mmol) and the mixture of compounds 41 and 42 (11 mg, 0.045 mmol) to provide after purification to separate to two analogs A-030 (2.0 mg, 0.02 mmol, 7%) (MW_{Calc}+H=697.36; MW_{Obs}=697.19) and A-031 (5.1 mg, 0.007 mmol, 16%). (MW_{Calc}+H=711.38; MW_{Obs}=711.20).

A-032 prepared in a similar fashion to A-001 starting with compound 18 (30 mg, 0.045 mmol) and commercially available tert-butyl (S)-3-allyl-3-aminopyrrolidine-1-carboxylate (16.3 mg, 0.072 mmol) to provide after purification A-032 (14.3 mg, 0.021 mmol, 46%) (MW_{Calc}+H=693.37; MW_{Obs}=693.36).

A-033 prepared in a similar fashion to A-001 starting with compound 18 (30 mg, 0.045 mmol) and commercially available tert-butyl (R)-3-allyl-3-aminopyrrolidine-1-carboxylate (16.3 mg, 0.072 mmol) to provide after purification A-033 (4.7 mg, 0.07 mmol, 15%) (MW_{Calc}+H=693.37; MW_{Obs}=693.14).

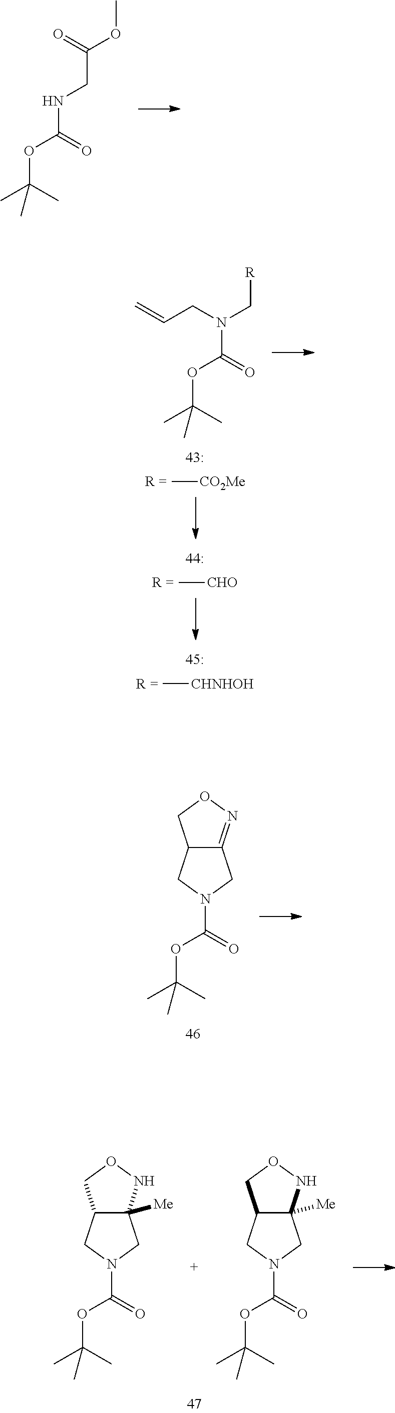

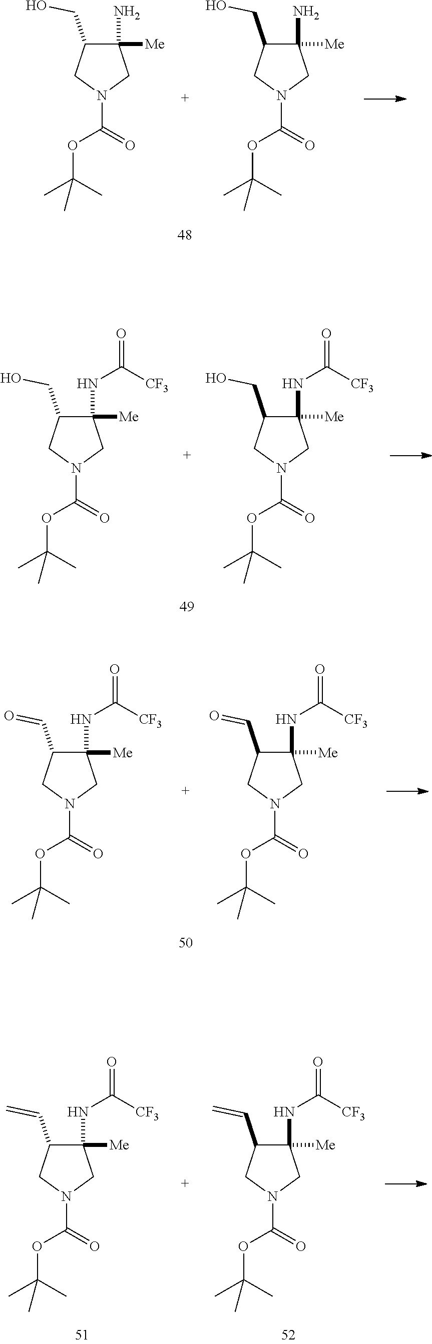

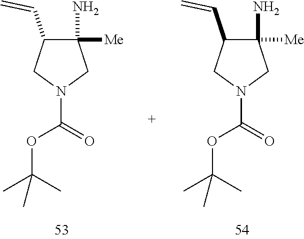 [SMILES: C=C[C@H]1CN(C(=O)OC(C)(C)C)C[C@@]1(C)N ; C=C[C@@H]1CN(C(=O)OC(C)(C)C)C[C@]1(C)N]

To a stirred solution of commercially available methyl (tert-butoxycarbonyl)glycinate (80 g, 422.8 mmol) in DMF (720 mL) was added allyl bromide (55.2 mL, 637.9 mmol) at room temperature upon which time the mixture was cooled to −20° C., and 60% sodium hydride (25.4 g, 634.1 mmol) was added portion wise maintaining the temperature below 0° C. followed by stirring for an additional 1 h at −20° C. The reaction mixture was slowly and carefully warmed to −2° C., and stirred for 2.5 h. The completed reaction was carefully quenched with the dropwise addition of sat. NH₄Cl (400 mL), followed by water (400 mL), and stirred at room temperature 30 min. The mixture was extracted EtOAc (2×500 mL ea), and the combined organic layers were washed with water (3×400 mL ea), 1:1 brine:water (400 mL), dried over Na₂SO₄, filtered and concentrated. The residue was azeotroped to dry with toluene (500 mL) to provide compound 43 (120 g, 377 mmol, 89%), which was used directly in next step without purification.

To a stirred solution of methyl N-allyl-N-(tert-butoxycarbonyl)glycinate (43, 86 g, 375.1 mmol) in DCM (1280 mL) at −78° C. was added 1.0 M DIBAL-H in DCM (506 ml, 506. mmol) portion wise maintaining the temperature below −70° C. followed by stirring for an addition 1.5 h at −73° C. The completed reaction was slowly quenched with methanol (10.32 mL, 255.1 mmol) dropwise maintaining the temperature below −70° C. followed by stirring for an additional 10 min. The completed reaction was warmed to 0° C. after which time 2 M sodium hydroxide (1440 mL, 2880.0 mmol) was slowly added after which time it was stirred for an additional 1 h. The layers were separated, and the aqueous layer was extracted with DCM (1 L). The combined organic layers were washed with water (2×1500 mL ea), washed with 1:1 water:brine (800 mL), dried over Na₂SO₄, filtered, and concentrated to dry to provide compound 44 (83.9 g, crude) which was used in the next step without purification.

To a stirred solution of tert-butyl allyl(2-oxoethyl)carbamate (44, 74.7 g, 374.9 mmol) in DCM (562 mL) and methanol (282 mL) at room temperature was added hydroxylamine hydrochloride (74.0 g, 1064.7 mmol) and sodium acetate (87 g, 1064.7 mmol) followed by stirring for 24 h. Water (1600 mL) was added to the completed reaction, the layers separated, and the aqueous layer was extracted with DCM (800 mL). The combined organic layers were washed with 1:1 water:brine (400 mL), dried over Na₂SO₄, concentrated, and dried under vacuum to provide compound 45 (80.39 g, 375 mmol, 100%), which was used without further purification.

To a stirred solution of 0.81 M sodium hypochlorite (880 ml, 712.6 mmol) in was added dropwise a solution of tert-butyl allyl(2-(hydroxyimino)ethyl)carbamate (45, 80 g, 373.4 mmol) in DCM (776 mL) maintaining the temperature below 25° C. The mixture was stirred for 1 h after which time water (960 mL) and DCM (320 mL) were added. The layers were separated, and aqueous layers were extracted with DCM (640 mL). The combined organic layers were washed with 1:1 water:brine (500 mL), dried over Na₂SO₄, filtered, and concentrated to dry. The residue was azeotroped with acetonitrile (2×300 mL), and then n-heptane (2×300 mL) upon which time a solid was formed. The solid was suspended in a mixture of EtOAc (15 mL) in n-heptane (300 mL), heated to 90° C., and stirred at 90° C. for 15 min after the solid dissolved into solution. The solution was cooled slowly to 0° C., and allowed to stand for 1 h. The resultant solid was filtered and washed with n-heptane (300 mL) to provide 50 g of crude product after drying under vacuum. The solid was re-crystalized using the same method described above to provide compound 46 (47.5 g, 224.0 mmol, 60%).

To a stirred solution of tert-butyl 3a,4-dihydro-3H-pyrrolo[3,4-c]isoxazole-5(6H)-carboxylate (46, 15 g, 70.67 mmol) in THF (102 mL) and toluene (102 mL) at −78° C. under a nitrogen atmosphere was added boron trifluoride etherate (10.23 mL, 80.71 mmol) followed by 3 M methylmagnesium bromide (28.3 mL, 84.81 mmol) in Et₂O maintaining the temperature below −70° C. After stirring for 1 h at −78° C. additional boron trifluoride etherate (8.96 mL, 70.67 mmol) and 3 M methylmagnesium bromide (23.56 mL, 70.67 mmol) were added sequentially followed by stirring −78° C. for 16 h. The completed reaction was warmed to 0° C., and slowly quenched with sat. NaHCO₃ (700 mL) with EtOAc (400 mL). The resultant layers were separated, and the aqueous layer was extracted with EtOAc (300 mL). The combined organic layers were washed with 1:1 water:brine (300 mL), dried over Na₂SO₄, filtered, and concentrated to provide crude product 47 (18.2 g), which was used in the next reaction without further purification. (MW_{Calc}+Na=241.15; MW_{Obs}=241.36).

To a stirred solution crude 47 (18.2 g, ca. 70.67 mmol) in THF (273 mL) under a N₂ atmosphere was added acetic acid (34 mL, 593.9 mmol) followed by cooling with an ice bath to ca. 8° C. Zinc powder (17.05 g, 260.7 mmol) was added two portions followed by allowing the reaction to warm to room temperature and stirred for 16 h. The completed reaction was filtered over a pad of Celite (30 g), eluted with EtOAc (500 mL). The filtrate was diluted with water (150 mL) followed by the addition of sodium bicarbonate (59.4 g, 706.7 mmol) with stirring. The mixture was stirred for 1 h and the layers separated. The aqueous layer was extracted with EtOAc (200 mL), and the combined organic layers were washed 1:1 water:brine (200 mL), dried over Na₂SO₄, filtered and concentrated to provide compound 48 (10.45 g, 45.4 mmol, 64%) as a racemic mixture without further purification. (MW_{Calc}+Na=253.16; MW_{Obs}=253.06).

To a stirred solution of tert-butyl (3S,4S)-3-amino-4-(hydroxymethyl)-3-methylpyrrolidine-1-carboxylate and its enantiomer (48, 1.6 g, 6.95 mmol) in DCM (22 mL) at 0° C. was added pyridine (1.686 ml, 20.84 mmol) followed by a dropwise addition of trifluoroacetic anhydride (1.08 ml, 7.64 mmol) over 5-min period. The reaction mixture was warmed to room temperature and stirred for 18 h. The completed reaction was quenched with sat. NaHCO₃ (30 mL), and stirred for 3 h. The resultant solution was extracted with MTBE (3×20 mL ea), and the combined organic layers were washed with brine (20 mL), dried over dried over Na₂SO₄, filtered and concentrated. The crude oil was purified over a Biotage SNAP column (25 g) eluting with 0-100% EtOAc in heptane (10 CV) to provide compound 49 (1.48 g, 4.54 mmol, 65%) as an oil after collection of the desired fractions, concentration and drying under vacuum.

To a stirred solution of tert-butyl (3S,4S)-4-(hydroxymethyl)-3-methyl-3-(2,2,2-trifluoroacetamido)-pyrrolidine-1-carboxylate (49, 2.0 g, 6.13 mmol) in DCM (30 mL) at 0° C. was added Dess-Martin periodinane (4.55 g, 10.73 mmol) and then warmed to room temperature. The reaction was stirred for 3 h after which time a solution of sodium thiosulfate 2-3 g in aq NaHCO₃ (30 mL) added with stirring for 30 min. The mixture was diluted with EtOAc (50 mL), the layers separated, and the organic layer was washed with sat. NaHCO₃ (10 mL), water (10 mL), and brine (10 mL). The organic layer was filtered over a plug of silica gel (20 g silica) eluting with EtOAc (20 mL), and the filtrate and concentrated followed by azeotroping to dryness with THF (2×20 mL ea). The crude aldehyde 50 was used in the next step.

To a stirred solution of (methyl)triphenylphosphonium bromide (4.93 g, 13.79 mmol) in THF (24 mL) at −8° C. under a N₂ atmosphere was added n-BuLi (6.74 ml, 13.48 mmol) over 5 min followed by stirring for 10 minutes. The resultant ylide was cooled to −78° C. followed by the dropwise addition of tert-butyl (3S,4S) and (3R,4R)-4-formyl-3-methyl-3-(2,2,2-trifluoroacetamido)pyrrolidine-1-carboxylate (50, ca 1.99 g, 6.13 mmol) in THF (12 mL) over 5 minutes. The reaction was stirred at −78° C. for an additional 5 min followed by slowly warming to room temperature and stirring for 3 h. The completed reaction was diluted with MTBE (10 mL) and silica gel (5 g) was added. The suspension was filtered over a pad of silica gel (5 g) eluting with MTBE (20 mL). The filtrate was first purified over a Biotage SNAP column (25 g) eluting with 0-100% EtOAc in n-heptane (10 CV) to provide a mixture of compounds 51 and 52 (880 mg) after collection of the desired fractions, concentration and drying under vacuum. The two enantiomers were separated using a 10×250 mm ChiralPak IC column at 35° C. eluting with a 40% methylene chloride in n-heptane with a 3 mL/min flow rate. The separation was performed by charging the column multiple times with approx. 50 mg each of the crude mixture, pooling the desired separated fractions to provide compound 51 (260 mg, 0.812 mmol, 13%) (MW_{Calc}+H=323.15; MW_{Obs}=323.15) as fraction 1, and compound 52 (260 mg, 0.812 mmol, 13%) (MW_{Calc}+H=323.15; MW_{Obs}=323.18) as fraction 2 after concentration and drying under vacuum.

To a stirred solution of tert-butyl (3R,4R)-3-methyl-3-(2,2,2-trifluoroacetamido)-4-vinylpyrrolidine-1-carboxylate (51, 235 mg, 0.729 mmol) in methanol (3 mL) was added 1 M sodium hydroxide (3.0 ml, 3.00 mmol) followed by warming in a closed vial at 45° C. for 24 h. The reaction was cooled to room temperature, concentrated and extracted with DCM (3×2 mL ea). The combined organic layers were washed with brine (2 mL), concentrated, and azeotroped to dry with toluene (2×2 mL) to provide compound 53 (ca 165 mg, 0.729 mmol, 100%) (MW_{Calc}+Na=249.17; MW_{Obs}=249.34).

Compound 54 (ca 165 mg, 0.729 mmol, 100%) was obtained in a similar fashion starting with 52 (235 mg, 0.729 mmol).

A-034 was prepared in a similar fashion to A-001 starting with compound 18 (13 mg, 0.020 mmol) and tert-butyl (3R,4R)-3-amino-3-methyl-4-vinylpyrrolidine-1-carboxylate (53, 7.1 mg, 0.031 mmol) to provide after purification A-034 (8.32 mg, 0.015 mmol, 53%) (MW_{Calc}+H=693.37; MW_{Obs}=693.34).

A-035 was prepared in a similar fashion to A-001 starting with compound 18 (13 mg, 0.020 mmol) and tert-butyl (3R,4R)-3-amino-3-methyl-4-vinylpyrrolidine-1-carboxylate (54, 7.1 mg, 0.031 mmol) to provide after purification A-035 (13.61 mg, 0.019 mmol, 86%) (MW_{Calc}+H=693.37; MW_{Obs}=693.35).

### Preparation of A-036

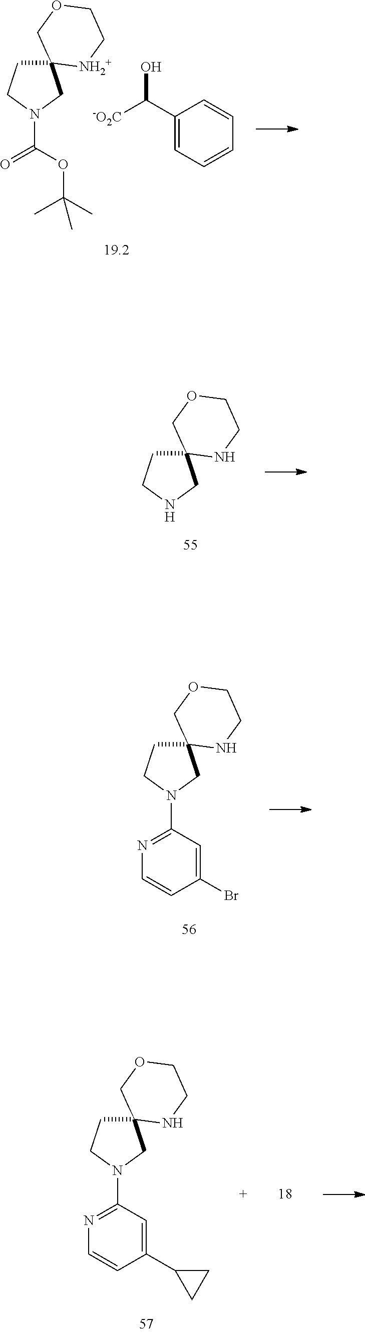

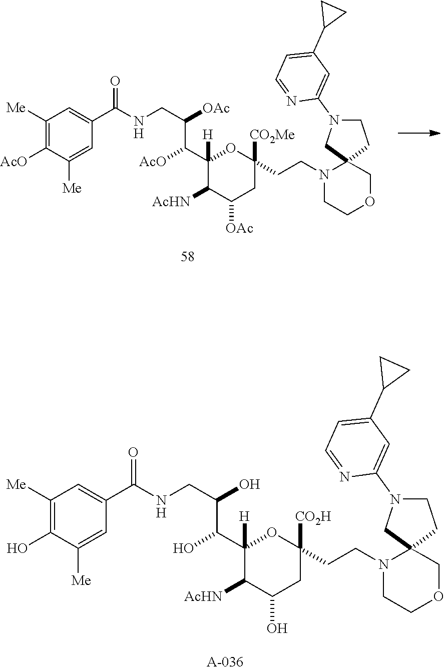

To a stirred slurry of tert-butyl (S)-9-oxa-2,6-diazaspiro[4.5]decane-2-carboxylate (S)-2-hydroxy-2-phenylacetate salt (19, 1.58 g, 4.005 mmol) in dichloromethane (100 mL) was add sat aq sodium bicarbonate (100 mL). The mixture was shaken for 5 min followed by separation of the layers. The organic layer was washed a second time with sat aq sodium bicarbonate. The combined aqueous layers were extracted with DCM (2×25 mL). The combined organic phases were concentrated under reduced pressure, and azeotroped to dry with dichloroethane (3×50 mL ea) to provide compound 55 (0.97 g, 4.01 mmol, 100%), which was used in the next steps without further purification.

To a stirred solution of (S)-9-oxa-2,6-diazaspiro[4.5]decane (55, 83 mg, 0.584 mmol) in THF (1.0 mL) was added triethylamine (0.814 μL, 5.837 mmol) and 4-bromo-2-fluoropyridine (308 mg, 1.751 mmol) followed by microwave heating to 120° C. for 8 hr. The cooled, completed reaction was directly purified by HPLC (ammonium hydroxide condition) to provide compound 56 (111 mg, 0.372 mmol, 64%) (MW_{Calc}+H=300.55; MW_{Obs}=300.10) after concentration of the desired fractions and drying under vacuum.

A stirred suspension of cyclopropylboronic acid (48.0 mg, 0.558 mmol) and potassium carbonate (185 mg, 1.34 mmol) in 1,4-dioxane (4.8 mL) and water (0.96 mL) was added (S)-2-(4-bromopyridin-2-yl)-9-oxa-2,6-diazaspiro[4.5]decane (56, 111 mg, 0.372 mmol) was degassed for 30 mins after which time Pd(PPh₃)₄ (43.0 mg, 0.037 mmol) was added. The mixture was degassed for an additional 20 mins at room temperature followed by stirring in the closed flask at 80° C. for 2 hr, and 100° C. for 16. The reaction was cooled to room temperature for by additional Pd(PPh₃)₄ (43.0 mg, 0.037 mmol) being added to the reaction, degassing for 20 min, and heating the closed flask 100° C. for 8 hr. The completed reaction was cooled to room temperature, filtered, and purified directly over a HPLC column to provide compound 57 (47.5 mg, 0.183 mmol, 49%) after concentration of the desired fractions and drying under vacuum.

To a stirred solution of (1R,2R)-1-((2R,3R,4S,6S)-3-acetamido-4-acetoxy-6-(methoxycarbonyl)-6-(2-oxoethyl)tetrahydro-2H-pyran-2-yl)-3-(4-acetoxy-3,5-dimethylbenzamido)propane-1,2-diyl diacetate (18, 28.6 mg, 0.043 mmol) in DCE (0.34 mL) was added acetic acid (18.5 μL, 0.323 mmol), and then (S)-2-(4-cyclopropylpyridin-2-yl)-9-oxa-2,6-diazaspiro[4.5]decane (57, 16.7 mg, 0.065 mmol). The reaction was stirred at room temperature for 1 h after which time dried 4A MS (100 mgs) followed by stirring for an additional 2 h. Sodium triacetoxyborohydride (18.2 mg, 0.086 mmol) was then added, and the reaction was stirred at room temperature for 16 h. The completed reaction was quenched with aq NaHCO₃ (5 mL), and extracted with EtOAc (3×20 mL ea). The combined organic layers were washed with brine and concentrated to dry to provide crude compound 58 that was used directly in the next reaction. (MW_{Calc}+H=908.42; MW_{Obs}=908.45).

To a stirred solution of (1R,2R)-1-((2R,3R,4S,6R)-3-acetamido-4-acetoxy-6-(2-((S)-2-(4-cyclopropylpyridin-2-yl)-9-oxa-2,6-diazaspiro[4.5]decan-6-yl)ethyl)-6-(methoxycarbonyl)tetrahydro-2H-pyran-2-yl)-3-(4-acetoxy-3,5-dimethylbenzamido)propane-1,2-diyl diacetate (58, ca 39 mg, 0.043 mmol) in methanol (2.6 mL) at 0° C. was added 1 N sodium hydroxide (0.73 mL, 0.731 mmol) followed by stirring at room temperature for 16 h. The reaction mixture was directly purified over a HPLC column to provide A-036 (4.0 mg, 0.06 mmol, 13%) (MW_{Calc}+H=726.37; MW_{Obs}=726.36) after concentration of the desired fractions and drying under vacuum.

### Preparation of A-037 to A-041

A-037 was prepared in a similar fashion to A-036 starting with compound 55 (24 mg, 0.169 mmol) and commercially available 2-fluoropyridine (49.2 mg, 0.506 mmol) to provide after purification A-037 (31.2 mg, 0.035 mmol, 40%) (MW_{Calc}+H=686.34; MW_{Obs}=6896.33).

A-038 was prepared in a similar fashion to A-036 starting with compound 55 (60 mg, 0.422 mmol) and commercially available 2-fluoro-4-methylpyridine (141 mg, 1.266 mmol) to provide after purification A-038 (10.1 mg, 0.014 mmol, 3% overall) (MW_{Calc}+H=700.35; MW_{Obs}=700.35).

A-039 was prepared in a similar fashion to A-036 starting with compound 55 (135 mg, 0.949 mmol) and commercially available 2-fluoro-4-(trifluoromethyl)pyridine (470 mg, 2.848 mmol) to provide after purification A-039 (12 mg, 0.016 mmol, 2% overall) (MW_{Calc}+H=754.32; MW_{Obs}=754.32).

A-040 was prepared in a similar fashion to A-036 starting with compound 55 (24 mg, 0.169 mmol) and commercially available 2-chloropyrazine (58 mg, 0.506 mmol) to provide after purification A-040 (24.88 mg, 0.036 mmol, 39%) (MW_{Calc}+H=687.33; MW_{Obs}=687.33).

A-041 was prepared in a similar fashion to A-036 starting with compound 55 (135 mg, 0.949 mmol) and commercially available 2-fluoro-N,N-dimethylpyridin-4-amine (58 mg, 0.506 mmol) to provide after purification A-041 (18.4 mg, 0.025 mmol, 46% overall) (MW_{Calc}+H=729.38; MW_{Obs}=729.37).

### Preparation of A-042

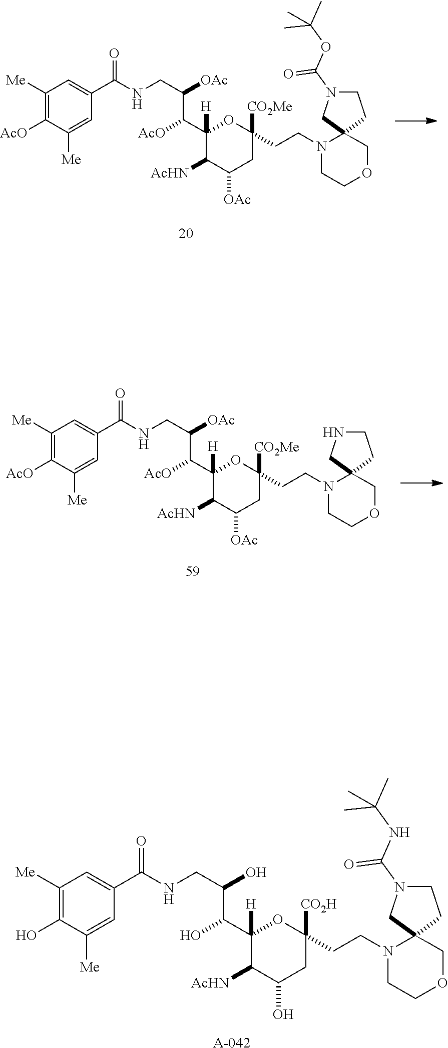

To a stirred solution of (1R,2R)-1-((2R,3R,4S,6R)-3-acetamido-4-acetoxy-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)-3-(4-acetoxy-3,5-dimethylbenzamido)propane-1,2-diyl diacetate (20, 862 mg, 0.967 mmol) in DCM (3735 mL) at 0° C. was added TFA (3.73 mL, 48.374 mmol) followed by stirring for 40 min maintaining the temperature at 0° C. The completed reaction was concentrated and azeotroped to dry with acetonitrile (2×20 mL ea), and vacuumed to dry to provide crude compound 59 (ca 765 mg, 0.967 mmol, 100%) (MW_{Calc}+H=790.36; MW_{Obs}=792.78) as an oil. The crude product was used in the next reaction without further purification.

To a stirred solution of (1R,2R)-1-((2R,3R,4S,6R)-6-(2-((S)-9-oxa-2,6-diazaspiro[4.5]decan-6-yl)ethyl)-3-acetamido-4-acetoxy-6-(methoxycarbonyl)tetrahydro-2H-pyran-2-yl)-3-(4-acetoxy-3,5-dimethylbenzamido)propane-1,2-diyl diacetate TFA salt (59, 20 mg, 0.025 mmol) in acetonitrile (1.0 mL) at room temperature was added triethylamine (0.025 mL, 0.177 mmol) followed by 2-isocyanato-2-methylpropane (5.0 mg, 0.051 mmol). The reaction was stirred for 1 h after which time it was quenched with sat. sodium bicarbonate (3 mL). The mixture was extracted with EtOAc (2×5 mL ea), and the combined organic layers were concentrated followed by azeotroping to dry with methanol (2×5 mL ea). The residue from the above reaction was subjected to a mixture of methanol (0.6 mL) and 1 N aqueous NaOH (0.4 mL) and stirred for 24 h at room temperature. The completed reaction was directly injected onto a reverse-phase HPLC column eluting with water/acetonitrile, to provide A-042 (11.3 mg, 0.016 mmol, 63%) (MW_{Calc}+H=708.38; MW_{Obs}=708.37) after collection of the desired fraction, concentration to dryness under vacuum.

### Preparation of A-043 to A-108

### Via Isocyanate or Isothiocyanate:

A-043 was prepared in a similar fashion to A-042 starting with compound 59 (150 mg, 0.190 mmol) and commercially available (S)-(1-isocyanatoethyl)benzene (36.3 mg, 0.07 mmol) to provide after purification A-043 (120 mg, 0.159 mmol, 84%) (MW_{Calc}+H=756.38; MW_{Obs}=756.52).

A-044 was prepared in a similar fashion to A-042 starting with compound 59 (25 mg, 0.190 mmol) and commercially available 1,1,1-trifluoro-2-isocyanatopropane (9.7 mg, 0.247 mmol) to provide after purification A-044 (8.2 mg, 0.011 mmol, 34%) (MW_{Calc}+H=748.33; MW_{Obs}=748.33).

A-045 was prepared in a similar fashion to A-042 starting with compound 59 (25 mg, 0.032 mmol) and commercially available isocyanatocyclohexane (5.0 mg, 0.04 mmol) to provide after purification A-045 (4.44 mg, 0.006 mmol, 27%) (MW_{Calc}+H=734.39; MW_{Obs}=734.39).

A-046 was prepared in a similar fashion to A-042 starting with compound 59 (27.5 mg, 0.035 mmol) and commercially available 1-(2-isocyanatopropan-2-yl)-3-(prop-1-en-2-yl)benzene (9.1 mg, 0.045 mmol) to provide after purification A-046 (1.94 mg, 0.002 mmol, 6.8%) (MW_{Calc}+H=810.42; MW_{Obs}=810.42).

A-047 was prepared in a similar fashion to A-042 starting with compound 59 (25 mg, 0.032 mmol) and commercially available (S)-3-(1-isocyanatoethyl)benzene-1-ylium (6.0 mg, 0.041 mmol) to provide after purification A-047 (12.9 mg, 0.017 mmol, 53%) (MW_{Calc}+H=756.38; MW_{Obs}=756.37).

A-048 was prepared in a similar fashion to A-042 starting with compound 59 (25 mg, 0.032 mmol) and commercially available 2-isocyanatopropane (3.0 mg, 0.035 mmol) to provide after purification A-048 (12.25 mg, 0.018 mmol, 55%) (MW_{Calc}+H=694.36; MW_{Obs}=694.36).

A-049 was prepared in a similar fashion to A-042 starting with compound 59 (25 mg, 0.032 mmol) and commercially available (3S,5S,7S)-1-isocyanatoadamantane (7.0 mg, 0.039 mmol) to provide after purification A-049 (12.14 mg, 0.015 mmol, 48%) (MW_{Calc}+H=786.42; MW_{Obs}=786.42).

A-050 was prepared in a similar fashion to A-042 starting with compound 59 (30 mg, 0.038 mmol) and commercially available 2-isocyanato-2,3-dimethylbutane (7.2 mg, 0.057 mmol) to provide after purification A-050 (8.7 mg, 0.012 mmol, 31%) (MW_{Calc}+H=736.41; MW_{Obs}=736.41).

A-051 was prepared in a similar fashion to A-042 starting with compound 59 (30 mg, 0.038 mmol) and commercially available 1,1-difluoro-4-isocyanatocyclohexane (6.6 mg, 0.041 mmol) to provide after purification A-051 (9 mg, 0.012 mmol, 56%) (MW_{Calc}+H=770.37; MW_{Obs}=770.37).

A-052 was prepared in a similar fashion to A-042 starting with compound 59 (25 mg, 0.032 mmol) and commercially available isothiocyanatocyclohexane (5.8 mg, 0.041 mmol) to provide after purification A-052 (13 mg, 0.018 mmol, 83%) (MW_{Calc}+H=750.37; MW_{Obs}=750.37).

A-053 was prepared in a similar fashion to A-042 starting with compound 59 (25 mg, 0.032 mmol) and commercially available 1-isocyanato-4-methylbenzene (5.0 mg, 0.038 mmol) to provide after purification A-053 (13 mg, 0.018 mmol, 83%) (MW_{Calc}+H=742.36; MW_{Obs}=742.6).

A-054 was prepared in a similar fashion to A-042 starting with compound 59 (27.5 mg, 0.035 mmol) and commercially available (1-isocyanatocyclopropyl)benzene (7.2 mg, 0.045 mmol) to provide after purification A-054 (0.8 mg, 0.001 mmol, 3%) (MW_{Calc}+H=768.38; MW_{Obs}=768.37).

A-055 was prepared in a similar fashion to A-042 starting with compound 59 (25 mg, 0.032 mmol) and commercially available 1-fluoro-4-(1-isocyanatocyclopentyl)benzene (8.4 mg, 0.041 mmol) to provide after purification A-055 (15.3 mg, 0.019 mmol, 75%) (MW_{Calc}+H=770.39; MW_{Obs}=770.5).

A-056 was prepared in a similar fashion to A-042 starting with compound 59 (89 mg, 0.113 mmol) and commercially available (S)-1-bromo-4-(1-isocyanatoethyl)benzene (33 mg, 0.146 mmol) to provide after purification A-056 (48.2 mg, 0.058 mmol, 52%) (MW_{Calc}+H=836.29; MW_{Obs}=836.3).

A-057 was prepared in a similar fashion to A-042 starting with compound 59 (25 mg, 0.032 mmol) and commercially available 1-fluoro-4-(1-isocyanatocyclopentyl)benzene (8.4 mg, 0.041 mmol) to provide after purification A-057 (15.3 mg, 0.019 mmol, 75%) (MW_{Calc}+H=814.40; MW_{Obs}=814.5).

A-058 was prepared in a similar fashion to A-042 starting with compound 59 (25 mg, 0.032 mmol) and commercially available isothiocyanatocyclopropane (4.1 mg, 0.041 mmol) to provide after purification A-058. (MW_{Calc}+H=708.32; MW_{Obs}=708.5)

### Via Carboxylic Acid Chloride:

A-059 was prepared in a similar fashion to A-042 starting with compound 59 (25 mg, 0.032 mmol) and commercially available methyl(phenyl)carbamic chloride (7.0 mg, 0.041 mmol) to provide after purification A-059 (11.6 mg, 0.016 mmol, 71%) (MW_{Calc}+H=742.36; MW_{Obs}=742.6).

A-060 was prepared in a similar fashion to A-042 starting with compound 59 (25 mg, 0.032 mmol) and commercially available cyclopentyl-1-carbonyl chloride (5.5 mg, 0.041 mmol) to provide after purification A-060 (13 mg, 0.018 mmol, 82%) (MW_{Calc}+H=705.37; MW_{Obs}=705.6).

A-061 was prepared in a similar fashion to A-042 starting with compound 59 (20 mg, 0.025 mmol) and commercially available isopropyl(methyl)carbamic chloride (4.5 mg, 0.033 mmol) to provide after purification A-061 (6.6 mg, 0.009 mmol, 33%) (MW_{Calc}+H=708.38; MW_{Obs}=708.6).

A-062 was prepared in a similar fashion to A-042 starting with compound 59 (25 mg, 0.032 mmol) and commercially available pyrrolidine-1-carbonyl chloride (5.5 mg, 0.041 mmol) to provide after purification A-062 (13.55 mg, 0.019 mmol, 83%) (MW_{Calc}+H=706.36; MW_{Obs}=706.6).

A-063 was prepared in a similar fashion to A-042 starting with compound 59 (20 mg, 0.025 mmol) and commercially available benzyl(methyl)carbamic chloride (6 mg, 0.033 mmol) to provide after purification A-063 (8.1 mg, 0.011 mmol, 44%) (MW_{Calc}+H=756.37; MW_{Obs}=756.6).

A-064 was prepared in a similar fashion to A-042 starting with compound 59 (25 mg, 0.032 mmol) and commercially available dimethylcarbamic chloride (4.4 mg, 0.041 mmol) to provide after purification A-064 (9.3 mg, 0.014 mmol, 44%) (MW_{Calc}+H=680.35; MW_{Obs}=680.6).

A-065 was prepared in a similar fashion to A-042 starting with compound 59 (71 mg, 0.090 mmol) and commercially available 4,4-difluoropiperidine-1-carbonyl chloride (21.4 mg, 0.117 mmol) to provide after purification A-065 (32.1 mg, 0.042 mmol, 46%) (MW_{Calc}+H=756.38; MW_{Obs}=756.6).

### Via Carboxylic Acid:

A-066 was prepared in a similar fashion to A-042 starting with compound 59 (25 mg, 0.032 mmol) and commercially available diethylcarbamic chloride (5.6 mg, 0.041 mmol) to provide after purification A-066 (5.8 mg, 0.008 mmol, 29%) (MW_{Calc}+H=708.38; MW_{Obs}=708.6).

A-067 was prepared in a similar fashion to A-042 starting with compound 59 (25 mg, 0.032 mmol), commercially available cyclohexanecarboxylic acid (5.5 mg, 0.039 mmol) along with HATU (24.0 mg, 0.063 mmol), triethylamine (22 uL, 0.158 mmol) in DMF (0.4 mL) for the first step to provide after hydrolysis and purification A-067 (13 mg, 0.018 mmol, 83%) (MW_{Calc}+H=719.38; MW_{Obs}=719.6).

A-068 was prepared in a similar fashion to A-067 starting with compound 59 (30 mg, 0.038 mmol), commercially available 2-phenylpropanoic acid (7 mg, 0.047 mmol) along with HATU (29 mg, 0.076 mmol), triethylamine (26 uL, 0.253 mmol) in DMF (1 mL) for the first step to provide after hydrolysis and purification A-068 (11.16 mg, 0.015 mmol, 45%) (MW_{Calc}+H=741.37; MW_{Obs}=741.7).

A-069 was prepared in a similar fashion to A-067 starting with compound 59 (25 mg, 0.032 mmol), commercially available 6,6-difluorospiro[3.3]heptane-2-carboxylic acid (7.2 mg, 0.041 mmol) along with HATU (24 mg, 0.041 mmol), triethylamine (44 uL, 0.316 mmol) in DMF (0.49 mL) for the first step to provide after hydrolysis and purification A-069 (9.9 mg, 0.013 mmol, 49%) (MW_{Calc}+H=767.36; MW_{Obs}=767.6).

A-070 was prepared in a similar fashion to A-067 starting with compound 59 (20 mg, 0.025 mmol), commercially available 2,2-difluorocyclohexane-1-carboxylic acid (5.4 mg, 0.033 mmol) along with HATU (19.2 mg, 0.051 mmol), triethylamine (35 uL, 0.253 mmol) in DMF (0.86 mL) for the first step to provide after hydrolysis and purification A-070 (5.81 mg, 0.0077 mmol, 28%) (MW_{Calc}+H=755.36; MW_{Obs}=755.6.

A-071 was prepared in a similar fashion to A-042 starting with compound 59 (20 mg, 0.025 mmol), commercially available 1-methylcyclohexane-1-carboxylic acid (4.7 mg, 0.033 mmol) along with HATU (19.2 mg, 0.051 mmol), triethylamine (18 uL, 0.126 mmol) in DMF (0.4 mL) for the first step to provide after hydrolysis and purification A-071 (7.5 mg, 0.010 mmol, 38%) (MW_{Calc}+H=733.40; MW_{Obs}=733.6).

A-072 was prepared in a similar fashion to A-042 starting with compound 59 (20 mg, 0.025 mmol), commercially available 3,3-dimethylbutanoic acid (5.9 mg, 0.051 mmol) along with HATU (19.2 mg, 0.051 mmol), triethylamine (35 uL, 0.253 mmol) in acetonitrile (1.0 mL) for the first step to provide after hydrolysis and purification A-072 (11 mg, 0.015 mmol, 61%) (MW_{Calc}+H=707.38; MW_{Obs}=707.52).

A-073 was prepared in a similar fashion to A-042 starting with compound 59 (25 mg, 0.032 mmol), commercially available 2-(tetrahydro-2H-pyran-4-yl)acetic acid (6.0 mg, 0.042 mmol) along with HATU (24 mg, 0.063 mmol), triethylamine (22 uL, 0.158 mmol) in DMF (0.85 mL) for the first step to provide after hydrolysis and purification A-073 (14.3 mg, 0.019 mmol, 89%) (MW_{Calc}+H=735.38; MW_{Obs}=735.6).

A-074 was prepared in a similar fashion to A-042 starting with compound 59 (30 mg, 0.038 mmol), commercially available 3,3-difluorocyclopentanecarboxylic acid (7.0 mg, 0.047 mmol) along with HATU (29 mg, 0.076 mmol), triethylamine (26 uL, 0.190 mmol) in DMF (1.03 mL) for the first step to provide after hydrolysis and purification A-074 (10 mg, 0.013 mmol, 42%) (MW_{Calc}+H=741.35; MW_{Obs}=741.6).

A-075 was prepared in a similar fashion to A-042 starting with compound 59 (30 mg, 0.038 mmol), commercially available 4,4-difluorocyclohexanecarboxylic acid (8.0 mg, 0.047 mmol) along with HATU (29 mg, 0.076 mmol), triethylamine (26 uL, 0.190 mmol) in DMF (1.03 mL) for the first step to provide after hydrolysis and purification A-075 (10 mg, 0.013 mmol, 41%) (MW_{Calc}+H=755.36; MW_{Obs}=755.6).

A-076 was prepared in a similar fashion to A-042 starting with compound 59 (30 mg, 0.038 mmol), commercially available 2-cyclohexylpropanoic acid (8.0 mg, 0.051 mmol) along with HATU (29 mg, 0.076 mmol), triethylamine (26 uL, 0.190 mmol) in DMF (1.03 mL) for the first step to provide after hydrolysis and purification A-076 (8.9 mg, 0.012 mmol, 37%) (MW_{Calc}+H=747.41; MW_{Obs}=747.7).

A-077 was prepared in a similar fashion to A-042 starting with compound 59 (30 mg, 0.038 mmol), commercially available 2-methyltetrahydrofuran-2-carboxylic acid (6.0 mg, 0.046 mmol) along with HATU (29 mg, 0.076 mmol), triethylamine (26 uL, 0.190 mmol) in DMF (1.03 mL) for the first step to provide after hydrolysis and purification A-077 (8.8 mg, 0.012 mmol, 37%) (MW_{Calc}+H=721.35; MW_{Obs}=721.6).

A-078 was prepared in a similar fashion to A-042 starting with compound 59 (25 mg, 0.032 mmol), commercially available 3-methylbut-2-enoic acid (4.0 mg, 0.040 mmol) along with HATU (24 mg, 0.063 mmol), triethylamine (22 uL, 0.158 mmol) in DMF (0.86 mL) for the first step to provide after hydrolysis and purification A-078 (1.2 mg, 0.0016 mmol, 5%) (MW_{Calc}+H=691.35; MW_{Obs}=691.6).

A-079 was prepared in a similar fashion to A-042 starting with compound 59 (30 mg, 0.038 mmol), commercially available 2-phenylacetic acid (7.0 mg, 0.040 mmol) along with HATU (29 mg, 0.076 mmol), triethylamine (26 uL, 0.190 mmol) in DMF (1.03 mL) for the first step to provide after hydrolysis and purification A-079 (10.5 mg, 0.014 mmol, 65%) (MW_{Calc}+H=727.35; MW_{Obs}=727.6).

A-080 was prepared in a similar fashion to A-042 starting with compound 59 (25 mg, 0.032 mmol), commercially available 3,3-dimethylcyclobutane-1-carboxylic acid (5.3 mg, 0.041 mmol) along with HATU (24 mg, 0.063 mmol), triethylamine (44 uL, 0.316 mmol) in DMF (0.49 mL) for the first step to provide after hydrolysis and purification A-080 (8.0 mg, 0.011 mmol, 40%) (MW_{Calc}+H=719.38; MW_{Obs}=719.6).

A-081 was prepared in a similar fashion to A-042 starting with compound 59 (30 mg, 0.038 mmol), commercially available 2-methyl-2-phenoxypropanoic acid (9 mg, 0.050 mmol) along with HATU (28.8 mg, 0.076 mmol), triethylamine (26 uL, 0.316 mmol) in DMF (1.03 mL) for the first step to provide after hydrolysis and purification A-081 (11.8 mg, 0.015 mmol, 49%) (MW_{Calc}+H=771.37; MW_{Obs}=771.7).

A-082 was prepared in a similar fashion to A-042 starting with compound 59 (30 mg, 0.038 mmol), commercially available 2-methoxypropanoic acid (5 mg, 0.048 mmol) along with HATU (28.8 mg, 0.076 mmol), triethylamine (26 uL, 0.316 mmol) in DMF (1.03 mL) for the first step to provide after hydrolysis and purification A-082 (6.0 mg, 0.0085 mmol, 25%) (MW_{Calc}+H=695.34; MW_{Obs}=695.6).

A-083 was prepared in a similar fashion to A-042 starting with compound 59 (30 mg, 0.038 mmol), commercially available 2-isobutoxyacetic acid (6 mg, 0.045 mmol) along with HATU (28.8 mg, 0.076 mmol), triethylamine (26 uL, 0.316 mmol) in DMF (1.03 mL) for the first step to provide after hydrolysis and purification A-083 (5.6 mg, 0.0078 mmol, 23%) (MW_{Calc}+H=723.37; MW_{Obs}=723.7).

A-084 was prepared in a similar fashion to A-042 starting with compound 59 (30 mg, 0.038 mmol), commercially available 2-isopropoxyacetic acid (6 mg, 0.051 mmol) along with HATU (28.8 mg, 0.076 mmol), triethylamine (26 uL, 0.316 mmol) in DMF (1.03 mL) for the first step to provide after hydrolysis and purification A-084 (6.4 mg, 0.09 mmol, 27%) (MW_{Calc}+H=709.36; MW_{Obs}=709.7).

A-085 was prepared in a similar fashion to A-042 starting with compound 59 (30 mg, 0.038 mmol), commercially available 2,2-dimethyl-2-methoxyacetic acid (6 mg, 0.051 mmol) along with HATU (28.8 mg, 0.076 mmol), triethylamine (26 uL, 0.316 mmol) in DMF (1.03 mL) for the first step to provide after hydrolysis and purification A-085 (9.3 mg, 0.013 mmol, 39%) (MW_{Calc}+H=709.36; MW_{Obs}=709.6).

A-086 was prepared in a similar fashion to A-042 starting with compound 59 (30 mg, 0.038 mmol), commercially available 2,6-dimethylbenzoic acid (7 mg, 0.047 mmol) along with HATU (28.8 mg, 0.076 mmol), triethylamine (26 uL, 0.316 mmol) in DMF (1.03 mL) for the first step to provide after hydrolysis and purification A-086 (8.1 mg, 0.011 mmol, 33%) (MW_{Calc}+H=741.36; MW_{Obs}=741.6).

A-087 was prepared in a similar fashion to A-042 starting with compound 59 (30 mg, 0.038 mmol), commercially available picolinic acid (6 mg, 0.049 mmol) along with HATU (28.8 mg, 0.076 mmol), triethylamine (26 uL, 0.316 mmol) in DMF (1.03 mL) for the first step to provide after hydrolysis and purification A-087 (4.3 mg, 0.006 mmol, 18%) (MW_{Calc}+H=723.38; MW_{Obs}=723.7).

A-088 was prepared in a similar fashion to A-042 starting with compound 59 (30 mg, 0.038 mmol), commercially available 2-(2,2,2-trifluoroethoxy)acetic acid (8 mg, 0.051 mmol) along with HATU (28.8 mg, 0.076 mmol), triethylamine (26 uL, 0.316 mmol) in DMF (1.03 mL) for the first step to provide after hydrolysis and purification A-088 (1.2 mg, 0.0016 mmol, 5%) (MW_{Calc}+H=749.32; MW_{Obs}=749.6).

A-089 was prepared in a similar fashion to A-042 starting with compound 59 (30 mg, 0.038 mmol), commercially available 3-(carboxy(cyclohexyl)methyl)benzene-1-ylium (11 mg, 0.051 mmol) along with HATU (28.8 mg, 0.076 mmol), triethylamine (26 uL, 0.316 mmol) in DMF (1.03 mL) for the first step to provide after hydrolysis and purification A-089 (9.9 mg, 0.012 mmol, 40%) (MW_{Calc}+H=809.43; MW_{Obs}=809.7).

A-090 was prepared in a similar fashion to A-042 starting with compound 59 (30 mg, 0.038 mmol), commercially available 2-(tert-butoxy)acetic acid (6 mg, 0.045 mmol) along with HATU (28.8 mg, 0.076 mmol), triethylamine (26 uL, 0.316 mmol) in DMF (1.03 mL) for the first step to provide after hydrolysis and purification A-090 (4.8 mg, 0.0066 mmol, 20%) (MW_{Calc}+H=723.38; MW_{Obs}=723.7).

A-091 was prepared in a similar fashion to A-042 starting with compound 59 (30 mg, 0.038 mmol), commercially available tetrahydro-2H-pyran-4-carboxylic acid (6 mg, 0.046 mmol) along with HATU (28.8 mg, 0.076 mmol), triethylamine (26 uL, 0.316 mmol) in DMF (1.03 mL) for the first step to provide after hydrolysis and purification A-091 (7.2 mg, 0.01 mmol, 30%) (MW_{Calc}+H=721.36; MW_{Ob}S=721.6).

A-092 was prepared in a similar fashion to A-042 starting with compound 59 (30 mg, 0.038 mmol), commercially available 2-(cyclopentyloxy)acetic acid (7 mg, 0.049 mmol) along with HATU (28.8 mg, 0.076 mmol), triethylamine (26 uL, 0.316 mmol) in DMF (1.03 mL) for the first step to provide after hydrolysis and purification A-092 (4.3 mg, 0.0058 mmol, 18%) (MW_{Calc}+H=735.38; MW_{Obs}=735.6).

A-093 was prepared in a similar fashion to A-042 starting with compound 59 (30 mg, 0.038 mmol), commercially available furan-2-carboxylic acid (5 mg, 0.045 mmol) along with HATU (28.8 mg, 0.076 mmol), triethylamine (26 uL, 0.316 mmol) in DMF (1.03 mL) for the first step to provide after hydrolysis and purification A-093 (7.4 mg, 0.011 mmol, 32%) (MW_{Calc}+H=703.31; MW_{Obs}=703.6).

A-094 was prepared in a similar fashion to A-042 starting with compound 59 (30 mg, 0.038 mmol), commercially available 1H-benzo[d]imidazole-5-carboxylic acid (8 mg, 0.049 mmol) along with HATU (28.8 mg, 0.076 mmol), triethylamine (26 uL, 0.316 mmol) in DMF (1.03 mL) for the first step to provide after hydrolysis and purification A-094 (2.8 mg, 0.0037 mmol, 12%) (MW_{Calc}+H=753.34; MW_{Obs}=753.34).

A-095 was prepared in a similar fashion to A-042 starting with compound 59 (30 mg, 0.038 mmol), commercially available 2-(1-hydroxyethyl)-1H-benzo[d]imidazole-5-carboxylic acid (10 mg, 0.048 mmol) along with HATU (28.8 mg, 0.076 mmol), triethylamine (26 uL, 0.316 mmol) in DMF (1.03 mL) for the first step to provide after hydrolysis and purification A-095 (9.3 mg, 0.012 mmol, 38%) (MW_{Calc}+H=797.36; MW_{Obs}=797.6).

A-096 was prepared in a similar fashion to A-042 starting with compound 59 (25 mg, 0.032 mmol), commercially available cyclopropane carboxylic acid (4 mg, 0.046 mmol) along with HATU (24 mg, 0.063 mmol), triethylamine (22 uL, 0.158 mmol) in DMF (0.86 mL) for the first step to provide after hydrolysis and purification A-096 (14.1 mg, 0.020 mmol, 90%) (MW_{Calc}+H=677.33; MW_{Obs}=677.6).

A-097 was prepared in a similar fashion to A-042 starting with compound 59 (25 mg, 0.032 mmol), commercially available pivalic acid (4 mg, 0.039 mmol) along with HATU (24 mg, 0.063 mmol), triethylamine (22 uL, 0.158 mmol) in DMF (0.86 mL) for the first step to provide after hydrolysis and purification A-097 (12 mg, 0.017 mmol, 75%) (MW_{Calc}+H=693.36; MW_{Obs}=693.6).

A-098 was prepared in a similar fashion to A-042 starting with compound 59 (25 mg, 0.032 mmol), commercially available 1-phenylcyclopropane-1-carboxylic acid (7 mg, 0.043 mmol) along with HATU (24 mg, 0.063 mmol), triethylamine (22 uL, 0.158 mmol) in DMF (0.86 mL) for the first step to provide after hydrolysis and purification A-098 (14.9 mg, 0.0020 mmol, 94%) (MW_{Calc}+H=753.37; MW_{Obs}=753.6).

A-099 was prepared in a similar fashion to A-042 starting with compound 59 (25 mg, 0.032 mmol), commercially available 4-methylbenzoic acid (6 mg, 0.044 mmol) along with HATU (24 mg, 0.063 mmol), triethylamine (22 uL, 0.158 mmol) in DMF (0.86 mL) for the first step to provide after hydrolysis and purification A-099 (14 mg, 0.019 mmol, 87%) (MW_{Calc}+H=727.35; MW_{Obs}=727.6).

A-100 was prepared in a similar fashion to A-042 starting with compound 59 (25 mg, 0.032 mmol), commercially available 3-methyloxetane-3-carboxylic acid (4.8 mg, 0.041 mmol) along with HATU (24 mg, 0.063 mmol), triethylamine (22 uL, 0.158 mmol) in DMF (0.86 mL) for the first step to provide after hydrolysis and purification A-100 (13.5 mg, 0.019 mmol, 86%) (MW_{Calc}+H=707.34; MW_{Obs}=707.6).

A-101 was prepared in a similar fashion to A-042 starting with compound 59 (25 mg, 0.032 mmol), commercially available benzoic acid (5 mg, 0.041 mmol) along with HATU (19.2 mg, 0.051 mmol), triethylamine (22 uL, 0.158 mmol) in DMF (0.73 mL) for the first step to provide after hydrolysis and purification A-101 (10.3 mg, 0.014 mmol, 66%) (MW_{Calc}+H=713.33; MW_{Obs}=713.6).

A-102 was prepared in a similar fashion to A-042 starting with compound 59 (25 mg, 0.032 mmol), commercially available 1-(trifluoromethyl)cyclohexane-1-carboxylic acid (8.1 mg, 0.041 mmol) along with HATU (24 mg, 0.063 mmol), triethylamine (88 uL, 0.632 mmol) in DMF (0.49 mL) for the first step to provide after hydrolysis and purification A-102 (7.8 mg, 0.010 mmol, 31%) (MW_{Calc}+H=787.37; MW_{Obs}=787.5).

A-103 was prepared in a similar fashion to A-042 starting with compound 59 (25 mg, 0.032 mmol), commercially available 1-methylcyclobutane-1-carboxylic acid (4.7 mg, 0.041 mmol) along with HATU (24 mg, 0.063 mmol), triethylamine (88 uL, 0.632 mmol) in DMF (0.49 mL) for the first step to provide after hydrolysis and purification A-103 (7.6 mg, 0.011 mmol, 34%) (MW_{Calc}+H=705.37; MW_{Obs}=705.5).

### Via Reductive Amination:

A-104 was prepared by an alternative fashion to A-042 starting with compound 59 (20 mg, 0.025 mmol), commercially available 3-methylbenzaldehyde (6.1 mg, 0.051 mmol) along with sodium triacetoxyborohydride (10.7 mg, 0.051 mmol) and acetic acid (12.2 uL, 0.202 mmol) in DCM (1 mL) for 1 h for the first step, quenching with saturated aqueous sodium bicarbonate, and extraction with ethyl acetate for the first step to provide after 1 N aqueous sodium hydroxide hydrolysis and purification A-104 (8.1 mg, 0.011 mmol, 45%) (MW_{Calc}+H=713.37; MW_{Obs}=713.6).

### Other Analogs:

A-105 and A-106 were prepared in a similar fashion to A-042 starting with the fully protected intermediate used to obtain A010 (54.0 mg, 0.067 mmol), which then provided A-105 (16.6 mg, 0.023 mmol, 34% overall yield) (MW_{Calc}+H=721.41; MW_{Obs}=721.5) and A-106 (15.7 mg, 0.022 mmol, 33% overall yield) (MW_{Calc}+H=721.41; MW_{Obs}=721.5) after separation of the diastereomers using a chiral reversed-phase HPLC column, concentration of the desired fractions, and drying under vacuum.

A-107 and A-108 were prepared in a similar fashion to A-042 starting with the fully protected intermediate used to obtain A-012 (54.0 mg, 0.067 mmol), which then provided A-107 (6.1 mg, 0.009 mmol, 64% overall yield) (MW_{Calc}+H=707.39; MW_{Obs}=707.5) and A-108 (5.7 mg, 0.0081 mmol, 62% overall yield) (MW_{Calc}+H=707.39; MW_{Obs}=707.5) after separation of the diastereomers using a chiral reversed-phase HPLC column, concentration of the desired fractions, and drying under vacuum.

### Preparation of A-109

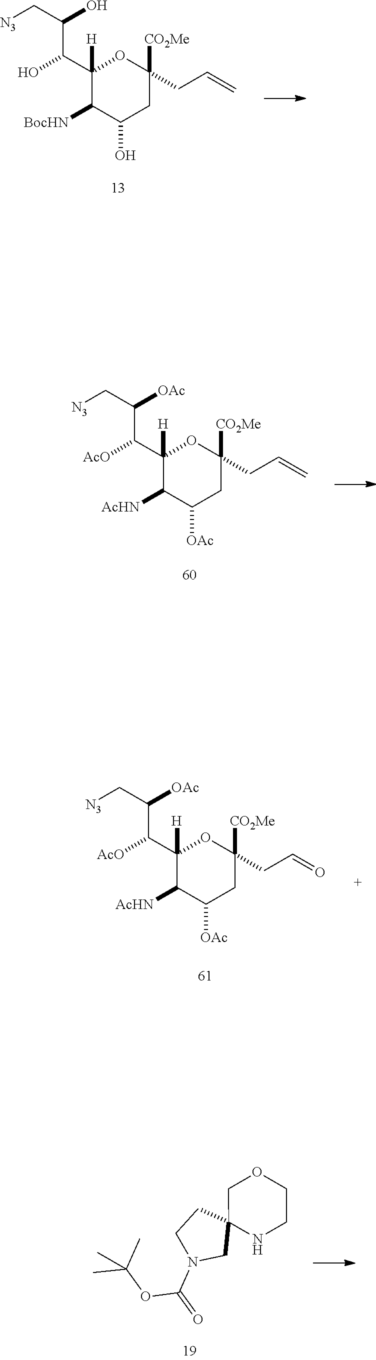

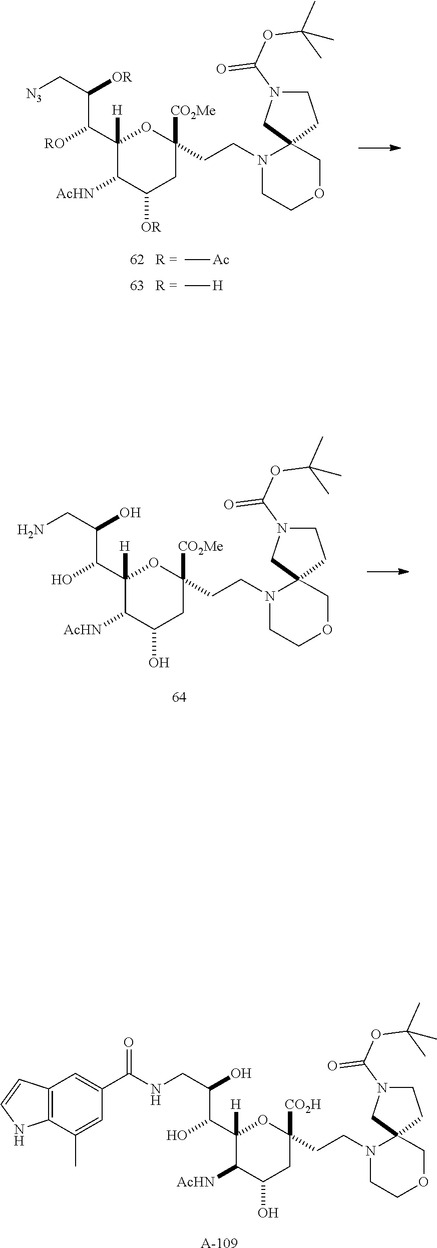

To a stirred solution of methyl (2R,4S,5R,6R)-2-allyl-6-((1R,2R)-3-azido-1,2-dihydroxypropyl)-5-((tert-butoxycarbonyl)amino)-4-hydroxytetrahydro-2H-pyran-2-carboxylate (13. 4.50 g, 10.45 mmol) in DCM (13.50 ml) was added TFA (8.05 ml, 104.54 mmol) at room temperature. The reaction was stirred for 3 h, after which time it was concentrated and azeotroped to dry with evaporated with toluene (3×25 mL ea). The residue obtained was dissolved in DCM (54.0 mL) followed by triethylamine (29.1 ml, 209.08 mmol), DMAP (0.128 g, 1.045 mmol) and then acetic anhydride (7.89 ml, 83.632 mmol) with stirring at room temperature. The reaction mixture was stirred for an additional 3 h, after which time it was quenched with saturated NaHCO₃ (30 mL) and extracted with EtOAc (5×40 mL ea). The combined organic layers were dried over Na2SO4, filtered, concentrated and the resulting residue was purified over a Biotage Ultra SNAP column (50 g) eluting with a gradient of 40% to 100% EtOAc in heptane (5 CV), then 0 to 10% MeOH in EtOAc (5 CV) to give compound 60 (3.90 g, 7.82 mmol, 75% yield) (MW_{Calc}+Na=521.2; MW_{Obs}=521.29) after collection of the desired fractions, concentration, and drying under vacuum.

To a stirred solution of (1R,2R)-1-((2R,3R,4S,6R)-3-acetamido-4-acetoxy-6-allyl-6-(methoxycarbonyl)tetrahydro-2H-pyran-2-yl)-3-azidopropane-1,2-diyl diacetate (60, 3.90 g, 7.824 mmol) in 1,4-dioxane (70.2 mL) and water (23.4 mL) was added 2,6-lutidine (1.822 ml, 15.647 mmol), osmium tetroxide (0.994 mL, 0.156 mmol), and sodium periodate (6.69 g, 31.295 mmol) at room temperature. The reaction mixture was stirred for 3 hr, after which time it was partitioned between EtOAc (80 mL) and water (40 mL). The aqueous layer was extracted with EtOAc (3×60 mL ea), and the combined organic layers were dried over Na2SO4, filtered, and concentrated. The residue was purified over a Biotage Ultra SNAP column (50 g) eluting with a gradient of 50% to 100% EtOAc in heptane (5 CV), then 0 to 20% MeOH in EtOAc (5 CV) to provided compound 61 (3.2 g, 6.39 mmol, 82% yield) (MW_{Calc}+Na=523.18; MW_{Obs}=523.32) after collection of the desired fractions, concentration, and drying under vacuum.

To a stirred solution of (1R,2R)-1-((2R,3R,4S,6S)-3-acetamido-4-acetoxy-6-(methoxycarbonyl)-6-(2-oxoethyl)tetrahydro-2H-pyran-2-yl)-3-azidopropane-1,2-diyl diacetate (61, 3.2 g, 6.394 mmol) in DCE (76 mL) was added acetic acid (2.56 ml, 44.759 mmol)) and tert-butyl (S)-9-oxa-2,6-diazaspiro[4.5]decane-2-carboxylate (19, 1.549 g, 6.394 mmol) followed by dried 4A MS (14 g) at room temperature. The reaction mixture was stirred at room temperature for 2 h after which time sodium triacetoxyborohydride (2.71 g, 12.788 mmol) added. The final reaction mixture was stirred at room temperature for 45 min, after which time it was diluted with EtOAc (50 mL) and filtered over a pad of Celite (50 g), and washed with EtOAc (3×20 mL). The filtrate was quenched with aq NaHCO₃ (40 mL), the layers separated, and the resulting aqueous layer was extracted with EtOAc (3×60 mL ea). The combined organic layers were washed with brine (50 mL), dried over Na₂SO₄, filtered, and concentrated.

The residue was purified over a Biotage Ultra SNAP column (50 g) eluting with a gradient of 30% to 100% EtOAc in heptane (5 CV), then 0 to 10% MeOH in EtOAc (5 CV) to give compound 62 (4.2 g, 5.78 mmol, 90% yield) after collection of the desired fractions, concentration, and drying under vacuum.

To a stirred solution of (1R,2R)-1-((2R,3R,4S,6R)-3-acetamido-4-acetoxy-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)-3-azidopropane-1,2-diyl diacetate (62, 4.2 g, 5.78 mmol) in MeOH (38.8 mL) was added K2CO3 (8.84 g, 63.941 mmol) at room temperature. The reaction mixture was stirred for 4 h, after which time the mixture was quenched with AcOH (5.49 mL, 95.912 mmol) and stirred for an additional 15 min. The mixture was concentrated and filtered through pad Celite (25 g) eluting with a mixture of 10:1 EtOAc/MeOH (75 mL). The resultant filtrate was concentrated and purified by an over a Biotage Ultra SNAP column (50 g) eluting with a gradient of 2 to 30% MeOH in DCM (10 CV) to give compound 63 (3.5 g, 5.83 mmol, 91% yield) (MW_{Calc}+H=601.31; MW_{Obs}=601.46) after collection of the desired fractions, concentration, and drying under vacuum.

To a stirred solution of tert-butyl (S)-6-(2-((2R,4S,5R,6R)-5-acetamido-6-((1R,2R)-3-azido-1,2-dihydroxypropyl)-4-hydroxy-2-(methoxycarbonyl)tetrahydro-2H-pyran-2-yl)ethyl)-9-oxa-2,6-diazaspiro[4.5]decane-2-carboxylate (63, 320 mg, 0.533 mmol) in wet-THF (3 mL with 45 uL H₂O, degassed and flushed with N₂) at room temperature was added 1 M trimethyl phosphine in THF solution (0.666 mL, 0.666 mmol). The reaction was stirred for 16 h, after which time it was concentrated maintaining the temperature below 35° C., and azeotroped with acetonitrile (3×10 mL ea), and dried under vacuum to provide the crude desired compound 64 (300 mg, 0.522 mmol, 98%) that was used in the next step without further purification.

To a stirred solution of tert-butyl (S)-6-(2-((2R,4S,5R,6R)-5-acetamido-6-((1R,2R)-3-amino-1,2-dihydroxypropyl)-4-hydroxy-2-(methoxycarbonyl)tetrahydro-2H-pyran-2-yl)ethyl)-9-oxa-2,6-diazaspiro[4.5]decane-2-carboxylate ((64), 19 mg, 0.033 mmol) in dimethylacetamide (1 mL) at room temperature was sequentially added HOBT-monohydrate (2.8 mg, 0.017 mmol), 7 methyl-1H-indole-5-carboxylic acid (8.7 mg, 0.05 mmol), EDC-HCl (10.5 mg, 0.055 mmol), and 1 M TEA (83 uL, 0.083 mmol in acetonitrile). The reaction mixture was stirred for 15 h to provide a majority of the desired fully protected intermediate by LCMS, which was directly treated with MeOH (0.3 mL) and 1M aq. NaOH (0.3 mL). The ensuing reaction was stirred for an additional 15 h at room temperature, after which time it was quenched with 2M aq formic acid (150 uL) and stirred for 15 min. The resulting mixture was purified using a reversed-phase C18 Xbridge HPLC column eluting with a water/acetonitrile gradient containing 0.1% NH₄OH to provide A-109 (6.1 mg, 0.013 mmol, 39%) (MW_{Calc}+H=718.36; MW_{Obs}=718.5) after collection of the desired fractions, concentration, and drying under vacuum.

### Preparation of A-110 to A-188

A-110 was prepared in a similar fashion to A-109 starting with compound 64 (16 mg, 0.028 mmol), commercially available benzo[d]thiazole-6-carboxylic acid (10 mg, 0.056 mmol) along with HOBT (2.1 mg, 0.014 mmol), triethylamine (97 uL, 0.097 mmol), and EDC-HCl (11.7 mg, 0.061 mmol) in dimethylacetamide (2 mL) for the first step to provide after hydrolysis and purification A-110 (2.7 mg, 0.004 mmol, 13%) (MW_{Calc}+H=722.30; MW_{Obs}=722.5).

A-111 was prepared in a similar fashion to A-109 starting with compound 64 (16 mg, 0.028 mmol), commercially available 1H-indazole-5-carboxylic acid (6.8 mg, 0.028 mmol) along with HOBT (2.1 mg, 0.014 mmol), 1 M triethylamine in THF (70 uL, 0.070 mmol), and EDC-HCl (8.8 mg, 0.046 mmol) in dimethylacetamide (1 mL) for the first step to provide after hydrolysis and purification A-111 (4.6 mg, 0.007 mmol, 23%) (MW_{Calc}+H=705.34; MW_{Obs}=705.19).

A-112 was prepared in a similar fashion to A-109 starting with compound 64 (12 mg, 0.021 mmol), commercially available 3-methyl-1H-indole-5-carboxylic acid (7.3 mg, 0.042 mmol) along with HOBT (3.2 mg, 0.021 mmol), triethylamine (8.7 uL, 0.063 mmol), and EDC-HCl (8.0 mg, 0.042 mmol) in dimethylacetamide (1 mL) for the first step to provide after hydrolysis and purification A-112 (4.5 mg, 0.0061 mmol, 29%) (MW_{Calc}+H=718.36; MW_{Obs}=718.41).

A-113 was prepared in a similar fashion to A-109 starting with compound 64 (12 mg, 0.021 mmol), commercially available 5-methyl-1H-pyrrole-3-carboxylic acid (2.6 mg, 0.021 mmol) along with HOBT (2.8 mg, 0.018 mmol), triethylamine (8.7 uL, 0.063 mmol), and EDC-HCl (8.0 mg, 0.042 mmol) in dimethylacetamide (1 mL) for the first step to provide after hydrolysis and purification A-113 (4.3 mg, 0.0064 mmol, 30%) (MW_{Calc}+H=668.35; MW_{Obs}=668.36).

A-114 was prepared in a similar fashion to A-109 starting with compound 64 (12 mg, 0.021 mmol), commercially available 1,5-naphthyridine-2-carboxylic acid (7.3 mg, 0.042 mmol) along with HOBT (3.2 mg, 0.019 mmol), triethylamine (15 uL, 0.104 mmol), and EDC-HCl (8.0 mg, 0.042 mmol) in dimethylacetamide (0.8 mL) for the first step to provide after hydrolysis and purification A-114 (4.5 mg, 0.0063 mmol, 30%) (MW_{Calc}+H=717.34; MW_{Obs}=717.31).

A-115 was prepared in a similar fashion to A-109 starting with compound 64 (12 mg, 0.021 mmol), commercially available 3,5-dibromo-4-hydroxybenzoic acid (12.4 mg, 0.042 mmol) along with HOBT (3.2 mg, 0.021 mmol), triethylamine (15 uL, 0.104 mmol), and EDC-HCl (8.0 mg, 0.042 mmol) in dimethylacetamide (0.8 mL) for the first step to provide after hydrolysis and purification A-115 (3.1 mg, 0.0037 mmol, 18%) (MW_{Calc}+H=839.15; MW_{Obs}=839.25).

A-116 was prepared in a similar fashion to A-109 starting with compound 64 (16 mg, 0.028 mmol), commercially available 3-bromobenzoic acid (8.4 mg, 0.042 mmol) along with HOBT (2.1 mg, 0.014 mmol), triethylamine (10 uL, 0.07 mmol), and EDC-HCl (8.8 mg, 0.046 mmol) in dimethylacetamide (0.26 mL) for the first step to provide after hydrolysis and purification A-116 (7.9 mg, 0.011 mmol, 40%) (MW_{Calc}+H=745.24; MW_{Obs}=745.3).

A-117 was prepared in a similar fashion to A-109 starting with compound 64 (16 mg, 0.028 mmol), commercially available 4-hydroxy-3-methylbenzoic acid (6.4 mg, 0.042 mmol) along with HOBT (2.1 mg, 0.014 mmol), triethylamine (10 uL, 0.07 mmol), and EDC-HCl (8.8 mg, 0.046 mmol) in dimethylacetamide (0.26 mL) for the first step to provide after hydrolysis and purification A-117 (7.2 mg, 0.010 mmol, 37%) (MW_{Calc}+H=695.35; MW_{Obs}=695.4).

A-118 was prepared in a similar fashion to A-109 starting with compound 64 (16 mg, 0.028 mmol), commercially available 4-methoxy-3,5-dimethylbenzoic acid (7.5 mg, 0.042 mmol) along with HOBT (2.1 mg, 0.014 mmol), triethylamine (10 uL, 0.07 mmol), and EDC-HCl (8.8 mg, 0.046 mmol) in dimethylacetamide (0.26 mL) for the first step to provide after hydrolysis and purification A-118 (7.5 mg, 0.010 mmol, 38%) (MW_{Calc}+H=723.38; MW_{Obs}=723.5).

A-119 was prepared in a similar fashion to A-109 starting with compound 64 (16 mg, 0.028 mmol), commercially available 4-amino-3,5-dimethylbenzoic acid (6.9 mg, 0.042 mmol) along with HOBT (2.1 mg, 0.014 mmol), triethylamine (10 uL, 0.07 mmol), and EDC-HCl (8.8 mg, 0.046 mmol) in dimethylacetamide (0.26 mL) for the first step to provide after hydrolysis and purification A-119 (5.6 mg, 0.008 mmol, 29%) (MW_{Calc}+H=708.38; MW_{Obs}=708.5).

A-120 was prepared in a similar fashion to A-109 starting with compound 64 (16 mg, 0.028 mmol), commercially available 3,4-dimethoxybenzoic acid (7.6 mg, 0.042 mmol) along with HOBT (2.1 mg, 0.014 mmol), triethylamine (10 uL, 0.07 mmol), and EDC-HCl (8.8 mg, 0.046 mmol) in dimethylacetamide (0.26 mL) for the first step to provide after hydrolysis and purification A-120 (4.4 mg, 0.006 mmol, 22%) (MW_{Calc}+H=725.35; MW_{Obs}=725.5).

A-121 was prepared in a similar fashion to A-109 starting with compound 64 (16 mg, 0.028 mmol), commercially available benzofuran-5-carboxylic acid (6.8 mg, 0.042 mmol) along with HOBT (1.9 mg, 0.014 mmol), triethylamine (10 uL, 0.07 mmol), and EDC-HCl (8.8 mg, 0.046 mmol) in dimethylacetamide (1.0 mL) for the first step to provide after hydrolysis and purification A-121 (4.4 mg, 0.006 mmol, 22%) (MW_{Calc}+H=705.33; MW_{Obs}=705.4).

A-122 was prepared in a similar fashion to A-109 starting with compound 64 (16 mg, 0.028 mmol), commercially available pyrazolo[1,5-a]pyrimidine-2-carboxylic acid (6.8 mg, 0.042 mmol) along with HOBT (1.9 mg, 0.014 mmol), triethylamine (10 uL, 0.07 mmol), and EDC-HCl (8.8 mg, 0.046 mmol) in dimethylacetamide (1.0 mL) for the first step to provide after hydrolysis and purification A-122 (5.5 mg, 0.008 mmol, 28%) (MW_{Calc}+H=706.34; MW_{Obs}=706.4).

A-123 was prepared in a similar fashion to A-109 starting with compound 64 (19 mg, 0.033 mmol), commercially available 4-hydroxy-3,5-dimethoxybenzoic acid (9.8 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-123 (9.3 mg, 0.013 mmol, 38%) (MW_{Calc}+H=741.35; MW_{Obs}=741.4).

A-124 was prepared in a similar fashion to A-109 starting with compound 64 (19 mg, 0.033 mmol), commercially available 1-methyl-1H-indazole-5-carboxylic acid (8.7 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-124 (6.8 mg, 0.009 mmol, 29%) (MW_{Calc}+H=719.36; MW_{Obs}=719.5).

A-125 was prepared in a similar fashion to A-109 starting with compound 64 (19 mg, 0.033 mmol), commercially available 3-methyl-1H-indazole-5-carboxylic acid (8.7 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-125 (6.4 mg, 0.009 mmol, 27%) (MW_{Calc}+H=719.36; MW_{Obs}=719.5).

A-126 was prepared in a similar fashion to A-109 starting with compound 64 (19 mg, 0.033 mmol), commercially available 1H-pyrazolo[3,4-b]pyridine-5-carboxylic acid (8.1 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-126 (2.3 mg, 0.003 mmol, 10%) (MW_{Calc}+H=706.34; MW_{Obs}=706.4).

A-127 was prepared in a similar fashion to A-109 starting with compound 64 (19 mg, 0.033 mmol), commercially available 3-methyl-1H-pyrazolo[3,4-b]pyridine-5-carboxylic acid (8.8 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-127 (9.8 mg, 0.014 mmol, 41%) (MW_{Calc}+H=720.35; MW_{Obs}=720.5).

A-128 was prepared in a similar fashion to A-109 starting with compound 64 (19 mg, 0.033 mmol), commercially available 3-fluoro-1H-indazole-5-carboxylic acid (8.9 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-128 (4.3 mg, 0.006 mmol, 18%) (MW_{Calc}+H=723.33; MW_{Obs}=723.5).

A-129 was prepared in a similar fashion to A-109 starting with compound 64 (19 mg, 0.033 mmol), commercially available 1H-indole-5-carboxylic acid (8.0 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-129 (7.3 mg, 0.010 mmol, 31%) (MW_{Calc}+H=704.35; MW_{Obs}=704.5).

A-130 was prepared in a similar fashion to A-109 starting with compound 64 (16.4 mg, 0.029 mmol), commercially available 7-methyl-1H-indazole-5-carboxylic acid (7.5 mg, 0.043 mmol) along with HOBT (2.4 mg, 0.014 mmol), 1 M triethylamine in THF (71 uL, 0.071 mmol), and EDC-HCl (9.0 mg, 0.047 mmol) in dimethylacetamide (1.5 mL) for the first step to provide after hydrolysis and purification A-130 (2.0 mg, 0.003 mmol, 10%) (MW_{Calc}+H=719.36; MW_{Obs}=719.4).

A-131 was prepared in a similar fashion to A-109 starting with compound 64 (16.4 mg, 0.029 mmol), commercially available 2-methylbenzo[d]thiazole-5-carboxylic acid (8.3 mg, 0.043 mmol) along with HOBT (2.4 mg, 0.014 mmol), 1 M triethylamine in THF (71 uL, 0.071 mmol), and EDC-HCl (9.0 mg, 0.047 mmol) in dimethylacetamide (1.5 mL) for the first step to provide after hydrolysis and purification A-131 (4.9 mg, 0.007 mmol, 23%) (MW_{Calc}+H=736.32; MW_{Obs}=736.4).

A-132 was prepared in a similar fashion to A-109 starting with compound 64 (12 mg, 0.021 mmol), commercially available 3-bromo-4-fluoro benzoic acid (9.2 mg, 0.042 mmol) along with HOBT (3.2 mg, 0.021 mmol), triethylamine (15 uL, 0.105 mmol), and EDC-HCl (6.0 mg, 0.042 mmol) in dimethylacetamide (0.5 mL) for the first step to provide after hydrolysis and purification A-132 (7.3 mg, 0.010 mmol, 46%) (MW_{Calc}+H=762.23; MW_{Obs}=763.3).

A-133 was prepared in a similar fashion to A-109 starting with compound 64 (12 mg, 0.021 mmol), commercially available 5-Bromo-1H-pyrazole-3-carboxylic acid (8.0 mg, 0.042 mmol) along with HOBT (3.2 mg, 0.021 mmol), triethylamine (15 uL, 0.105 mmol), and EDC-HCl (6.0 mg, 0.042 mmol) in dimethylacetamide (0.5 mL) for the first step to provide after hydrolysis and purification A-133 (7.0 mg, 0.010 mmol, 46%) (MW_{Calc}+H=735.23; MW_{Obs}=735.3).

A-134 was prepared in a similar fashion to A-109 starting with compound 64 (25 mg, 0.044 mmol), commercially available 2-(hydroxymethyl)-7-methyl-1H-indole-5-carboxylic acid (13.4 mg, 0.065 mmol) along with HATU (33.1 mg, 0.087 mmol), triethylamine (61 uL, 0.435 mmol) in DCM (0.56 mL) for the first step to provide after hydrolysis and purification A-134 (3.7 mg, 0.005 mmol, 11%) (MW_{Calc}+H=748.38; MW_{Obs}=748.5).

A-135 was prepared in a similar fashion to A-109 starting with compound 64 (16 mg, 0.028 mmol), commercially available 1H-benzo[d]imidazole-5-carboxylic acid (6.8 mg, 0.042 mmol) along with HOBT (2.1 mg, 0.014 mmol), 1 M triethylamine in THF (70 uL, 0.070 mmol), and EDC-HCl (8.8 mg, 0.046 mmol) in dimethylacetamide (1.0 mL) for the first step to provide after hydrolysis and purification A-135 (5.5 mg, 0.008 mmol, 28%) (MW_{Calc}+H=705.35; MW_{Obs}=705.5).

A-136 was prepared in a similar fashion to A-109 starting with compound 64 (14 mg, 0.024 mmol), commercially available 1,3-dimethyl-1H-pyrazole-4-carboxylic acid (6.8 mg, 0.049 mmol) along with HOBT (3.7 mg, 0.024 mmol), 10% triethylamine in THF (102 uL, 0.073 mmol), and EDC-HCl (9.3 mg, 0.049 mmol) in dimethylacetamide (0.5 mL) for the first step to provide after hydrolysis and purification A-136 (2.7 mg, 0.0039 mmol, 16%) (MW_{Calc}+H=683.36; MW_{Obs}=683.47).

A-137 was prepared in a similar fashion to A-109 starting with compound 64 (14 mg, 0.024 mmol), commercially available 2,3-dihydrobenzofuran-5-carboxylic acid (8 mg, 0.049 mmol) along with HOBT (3.7 mg, 0.024 mmol), 10% triethylamine in THF (102 uL, 0.073 mmol), and EDC-HCl (9.3 mg, 0.049 mmol) in dimethylacetamide (0.5 mL) for the first step to provide after hydrolysis and purification A-137 (6.0 mg, 0.0085 mmol, 35%) (MW_{Calc}+H=705.33; MW_{Obs}=707.52).

A-138 was prepared in a similar fashion to A-109 starting with compound 64 (12 mg, 0.021 mmol), commercially available 3-methyl-4-oxo-3,4-dihydroquinazoline-7-carboxylic acid (2.6 mg, 0.021 mmol) along with HOBT (2.6 mg, 0.021 mmol), triethylamine (8.7 uL, 0.063 mmol), and EDC-HCl (8 mg, 0.042 mmol) in dimethylacetamide (0.8 mL) for the first step to provide after hydrolysis and purification A-138 (3.3 mg, 0.0043 mmol, 20%) (MW_{Calc}+H=747.35; MW_{Obs}=747.38).

A-139 was prepared in a similar fashion to A-109 starting with compound 64 (12 mg, 0.021 mmol), commercially available 2-methyl-1H-imidazole-4-carboxylic acid (8.5 mg, 0.042 mmol) along with HOBT (3.2 mg, 0.021 mmol), triethylamine (8.7 uL, 0.063 mmol), and EDC-HCl (8 mg, 0.042 mmol) in dimethylacetamide (0.5 mL) for the first step to provide after hydrolysis and purification A-139 (5.1 mg, 0.0076 mmol, 36%) (MW_{Calc}+H=669.34; MW_{Obs}=669.65).

A-140 was prepared in a similar fashion to A-109 starting with compound 64 (12 mg, 0.021 mmol), commercially available 2,5-dimethyloxazole-4-carboxylic acid (3.0 mg, 0.042 mmol) along with HOBT (3.2 mg, 0.021 mmol), triethylamine (8.7 uL, 0.063 mmol), and EDC-HCl (8 mg, 0.042 mmol) in dimethylacetamide (0.5 mL) for the first step to provide after hydrolysis and purification A-140 (4.9 mg, 0.0071 mmol, 34%) (MW_{Calc}+H=684.34; MW_{Obs}=684.64).

A-141 was prepared in a similar fashion to A-109 starting with compound 64 (12 mg, 0.021 mmol), commercially available 4-carbamoyl-3,5-dimethylbenzoic acid (8 mg, 0.041 mmol) along with HOBT (3.2 mg, 0.021 mmol), triethylamine (8.7 uL, 0.063 mmol), and EDC-HCl (8 mg, 0.042 mmol) in dimethylacetamide (0.8 mL) for the first step to provide after hydrolysis and purification A-141 (5.3 mg, 0.0072 mmol, 34%) (MW_{Calc}+H=736.37; MW_{Obs}=736.42).

A-142 was prepared in a similar fashion to A-109 starting with compound 64 (12 mg, 0.021 mmol), commercially available 4-(1H-1,2,4-triazol-5-yl)benzoic acid (7.9 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-142 (5.6 mg, 0.0076 mmol, 36%) (MW_{Calc}+H=732.35; MW_{Obs}=732.49).

A-143 was prepared in a similar fashion to A-109 starting with compound 64 (12 mg, 0.021 mmol), commercially available 4-(4-Methylpiperazin-1-yl)benzoic acid (9.2 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-143 (7.1 mg, 0.0093 mmol, 44%) (MW_{Calc}+H=763.42; MW_{Obs}=763.68).

A-144 was prepared in a similar fashion to A-109 starting with compound 64 (16 mg, 0.028 mmol), commercially available 3-methyl-4-(trifluoromethyl)benzoic acid (8.5 mg, 0.042 mmol) along with HOBT (2.1 mg, 0.014 mmol), triethylamine (10 uL, 0.070 mmol) in DMA (0.5 mL) for the first step to provide after hydrolysis and purification A-144 (6.3 mg, 0.008 mmol, 32%) (MW_{Calc}+H=747.34; MW_{Obs}=747.4).

A-145 was prepared in a similar fashion to A-109 starting with compound 64 (16 mg, 0.028 mmol), commercially available 4-methyl-3-(trifluoromethyl)benzoic acid (8.5 mg, 0.042 mmol) along with HOBT (2.1 mg, 0.014 mmol), triethylamine (10 uL, 0.070 mmol) in DMA (0.5 mL) for the first step to provide after hydrolysis and purification A-145 (8.6 mg, 0.012 mmol, 44%) (MW_{Calc}+H=747.34; MW_{Obs}=747.4).

A-146 was prepared in a similar fashion to A-109 starting with compound 64 (16 mg, 0.028 mmol), commercially available 3-hydroxy-4-(trifluoromethyl)benzoic acid (8.6 mg, 0.042 mmol) along with HOBT (2.1 mg, 0.014 mmol), triethylamine (10 uL, 0.070 mmol) in DMA (0.5 mL) for the first step to provide after hydrolysis and purification A-146 (6.6 mg, 0.009 mmol, 34%) (MW_{Calc}+H=749.32; MW_{Obs}=749.4).

A-147 was prepared in a similar fashion to A-109 starting with compound 64 (16 mg, 0.028 mmol), commercially available 3-hydroxy-4-(trifluoromethyl)benzoic acid (8.6 mg, 0.042 mmol) along with HOBT (2.1 mg, 0.014 mmol), triethylamine (10 uL, 0.070 mmol) in DMA (0.5 mL) for the first step to provide after hydrolysis and purification A-147 (7.1 mg, 0.009 mmol, 36%) (MW_{Calc}+H=749.32; MW_{Obs}=749.4).

A-148 was prepared in a similar fashion to A-109 starting with compound 64 (16 mg, 0.028 mmol), commercially available 3,4,5-trimethoxybenzoic acid (8.9 mg, 0.042 mmol) along with HOBT (2.1 mg, 0.014 mmol), triethylamine (10 uL, 0.070 mmol) in DMA (0.5 mL) for the first step to provide after hydrolysis and purification A-148 (5.7 mg, 0.008 mmol, 29%) (MW_{Calc}+H=755.37; MW_{Obs}=755.5).

A-149 was prepared in a similar fashion to A-109 starting with compound 64 (16 mg, 0.028 mmol), commercially available 4-fluoro-3-methylbenzoic acid (6.4 mg, 0.042 mmol) along with HOBT (2.1 mg, 0.014 mmol), triethylamine (10 uL, 0.070 mmol) in DMA (0.5 mL) for the first step to provide after hydrolysis and purification A-149 (7.6 mg, 0.011 mmol, 39%) (MW_{Calc}+H=697.34; MW_{Obs}=697.4).

A-150 was prepared in a similar fashion to A-109 starting with compound 64 (16 mg, 0.028 mmol), commercially available 4-methyl-3-oxo-3,4-dihydro-2H-benzo[b][1,4]oxazine-6-carboxylic acid (8.7 mg, 0.042 mmol) along with HOBT (3.7 mg, 0.028 mmol), triethylamine (10 uL, 0.070 mmol) in DMA (1.0 mL) for the first step to provide after hydrolysis and purification A-150 (7.6 mg, 0.011 mmol, 39%) (MW_{Calc}+H=750.35; MW_{Obs}=750.5).

A-151 was prepared in a similar fashion to A-109 starting with compound 64 (19 mg, 0.033 mmol), commercially available 7-methyl-1H-indole-5-carboxylic acid (8.9 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-151 (7.0 mg, 0.010 mmol, 30%) (MW_{Calc}+H=705.34; MW_{Obs}=705.5).

A-152 was prepared in a similar fashion to A-109 starting with compound 64 (19 mg, 0.033 mmol), commercially available benzo[d]oxazole-5-carboxylic acid (8.1 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-152 (8.3 mg, 0.011 mmol, 35%) (MW_{Calc}+H=724.34; MW_{Obs}=724.5).

A-153 was prepared in a similar fashion to A-109 starting with compound 64 (19 mg, 0.033 mmol), commercially available benzo[c][1,2,5]thiadiazole-5-carboxylic acid (8.1 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-153 (4.9 mg, 0.007 mmol, 21%) (MW_{Calc}+H=723.30; MW_{Obs}=723.4).

A-154 was prepared in a similar fashion to A-109 starting with compound 64 (12 mg, 0.021 mmol), commercially available 1,5-Dimethyl-1H-pyrazole-3-carboxylic acid (5.9 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-154 (7.6 mg, 0.011 mmol, 53%) (MW_{Calc}+H=683.36; MW_{Obs}=683.5).

A-155 was prepared in a similar fashion to A-109 starting with compound 64 (12 mg, 0.021 mmol), commercially available 3-ethylbenzoic acid (6.3 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-155 (7.2 mg, 0.010 mmol, 49%) (MW_{Calc}+H=693.37; MW_{Obs}=693.5).

A-156 was prepared in a similar fashion to A-109 starting with compound 64 (12 mg, 0.021 mmol), commercially available 5-methyl-1h-pyrazole-3-carboxylic acid (5.3 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-156 (6.2 mg, 0.009 mmol, 44%) (MW_{Calc}+H=669.34; MW_{Obs}=669.5).

A-157 was prepared in a similar fashion to A-109 starting with compound 64 (12 mg, 0.021 mmol), commercially available 5-chloro-1H-pyrazole-3-carboxylic acid (6.1 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-157 (6.4 mg, 0.009 mmol, 45%) (MW_{Calc}+H=689.28; MW_{Obs}=689.3).

A-158 was prepared in a similar fashion to A-109 starting with compound 64 (12 mg, 0.021 mmol), commercially available 5-Methylfuran-3-carboxylic acid (5.3 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-158 (7.8 mg, 0.012 mmol, 56%) (MW_{Calc}+H=669.33; MW_{Obs}=669.4).

A-159 was prepared in a similar fashion to A-109 starting with compound 64 (12 mg, 0.021 mmol), commercially available 3,5-Dichloro-4-fluorobenzoic acid (8.7 mg, 0.042 mmol) along with HOBT (3.2 mg, 0.021 mmol), triethylamine (15 uL, 0.104 mmol), and EDC-HCl (8 mg, 0.042 mmol) in dimethylacetamide (0.8 mL) for the first step to provide after hydrolysis and purification A-159 (4.6 mg, 0.006 mmol, 29%) (MW_{Calc}+H=751.24; MW_{Obs}=751.3).

A-160 was prepared in a similar fashion to A-109 starting with compound 64 (16 mg, 0.028 mmol), commercially available 5-methyl-2-(trifluoromethyl)furan-3-carboxylic acid (11.9 mg, 0.061 mmol) along with HOBT (2.1 mg, 0.014 mmol), 1 M triethylamine in THF (97 uL, 0.097 mmol), and EDC-HCl (11.7 mg, 0.055 mmol) in acetonitrile (2 mL) for the first step to provide after hydrolysis and purification A-160 (4.9 mg, 0.007 mmol, 24%) (MW_{Calc}+H=737.32; MW_{Obs}=737.5).

A-161 was prepared in a similar fashion to A-109 starting with compound 64 (19 mg, 0.028 mmol), commercially available 1H-pyrazole-4-carboxylic acid (5.6 mg, 0.061 mmol) along with HOBT (2.8 mg, 0.014 mmol), 1 M triethylamine in THF (83 uL, 0.083 mmol), and EDC-HCl (10.4 mg, 0.055 mmol) in DMA (1.5 mL) for the first step to provide after hydrolysis and purification A-161 (10.2 mg, 0.016 mmol, 56%) (MW_{Calc}+H=655.33; MW_{Obs}=655.5).

A-162 was prepared in a similar fashion to A-109 starting with compound 64 (12 mg, 0.021 mmol), commercially available (E)-3-methylhex-2-enoic acid (5.4 mg, 0.042 mmol) along with HOBT (3.2 mg, 0.021 mmol), triethylamine (8.7 uL, 0.063 mmol), and EDC-HCl (8 mg, 0.042 mmol) in dimethylacetamide (1.9 mL) for the first step to provide after hydrolysis and purification A-162 (3.1 mg, 0.004 mmol, 20%) (MW_{Calc}+H=671.38; MW_{Obs}=671.48).

A-163 was prepared in a similar fashion to A-109 starting with compound 64 (12 mg, 0.021 mmol), commercially available (2E,4E)-hexa-2,4-dienoic acid (4.7 mg, 0.042 mmol) along with HOBT (3.2 mg, 0.021 mmol), triethylamine (8.7 uL, 0.063 mmol), and EDC-HCl (8 mg, 0.042 mmol) in dimethylacetamide (1.9 mL) for the first step to provide after hydrolysis and purification A-163 (3.7 mg, 0.0055 mmol, 26%) (MW_{Calc}+H=655.35; MW_{Obs}=655.39).

A-164 was prepared in a similar fashion to A-109 starting with compound 64 (12 mg, 0.021 mmol), commercially available 3,5-dimethyl-4-(methylcarbamoyl)benzoic acid (9.0 mg, 0.043 mmol) along with HOBT (3.2 mg, 0.021 mmol), triethylamine (8.7 uL, 0.063 mmol), and EDC-HCl (8 mg, 0.042 mmol) in dimethylacetamide (0.8 mL) for the first step to provide after hydrolysis and purification A-164 (3.2 mg, 0.0042 mmol, 20%) (MW_{Calc}+H=750.39; MW_{Obs}=750.53).

A-165 was prepared in a similar fashion to A-109 starting with compound 64 (14 mg, 0.024 mmol), commercially available 4-cyano-3-fluorobenzoic acid (8.1 mg, 0.049 mmol) along with HOBT (3.7 mg, 0.024 mmol), 10% triethylamine in THF (102 uL, 0.073 mmol), and EDC-HCl (9.3 mg, 0.049 mmol) in dimethylacetamide (0.5 mL) for the first step to provide after hydrolysis and purification A-165 (4.4 mg, 0.0061 mmol, 25%) (MW_{Calc}+H=720.34; MW_{Obs}=720.54).

A-166 was prepared in a similar fashion to A-109 starting with compound 64 (16 mg, 0.028 mmol), commercially available 3,5-difluoro-4-(trifluoromethyl)benzoic acid (9.4 mg, 0.042 mmol) along with HOBT (2.1 mg, 0.014 mmol), triethylamine (9.7 uL, 0.070 mmol), and EDC-HCl (8.8 mg, 0.046 mmol) in dimethylacetamide (1.5 mL) for the first step to provide after hydrolysis and purification A-166 (4.6 mg, 0.006 mmol, 23%) (MW_{Calc}+H=769.30; MW_{Obs}=769.4).

A-167 was prepared in a similar fashion to A-109 starting with compound 64 (16 mg, 0.028 mmol), commercially available 3,4-bis(trifluoromethyl)benzoic acid (10.8 mg, 0.042 mmol) along with HOBT (2.1 mg, 0.014 mmol), triethylamine (9.7 uL, 0.070 mmol), and EDC-HCl (8.8 mg, 0.046 mmol) in dimethylacetamide (1.5 mL) for the first step to provide after hydrolysis and purification A-167 (6.6 mg, 0.008 mmol, 34%) (MW_{Calc}+H=801.31; MW_{Obs}=801.4).

A-168 was prepared in a similar fashion to A-109 starting with compound 64 (16 mg, 0.028 mmol), commercially available 3,5-bis(trifluoromethyl)benzoic acid (10.8 mg, 0.042 mmol) along with HOBT (2.1 mg, 0.014 mmol), triethylamine (9.7 uL, 0.070 mmol), and EDC-HCl (8.8 mg, 0.046 mmol) in dimethylacetamide (1.5 mL) for the first step to provide after hydrolysis and purification A-168 (6.4 mg, 0.008 mmol, 33%) (MW_{Calc}+H=801.31; MW_{Obs}=801.4).

A-169 was prepared in a similar fashion to A-109 starting with compound 64 (16 mg, 0.028 mmol), commercially available 3-((trifluoromethyl)thio)benzoic acid (9.3 mg, 0.042 mmol) along with HOBT (2.1 mg, 0.014 mmol), triethylamine (9.7 uL, 0.070 mmol), and EDC-HCl (8.8 mg, 0.046 mmol) in dimethylacetamide (1.5 mL) for the first step to provide after hydrolysis and purification A-169 (7.2 mg, 0.009 mmol, 37%) (MW_{Calc}+H=765.29; MW_{Obs}=765.4).

A-170 was prepared in a similar fashion to A-109 starting with compound 64 (16 mg, 0.028 mmol), commercially available 1-methyl-6-oxo-1,6-dihydropyridine-3-carboxylic acid (6.4 mg, 0.042 mmol) along with HOBT (2.1 mg, 0.014 mmol), triethylamine (9.7 uL, 0.070 mmol), and EDC-HCl (8.8 mg, 0.046 mmol) in dimethylacetamide (1.5 mL) for the first step to provide after hydrolysis and purification A-170 (6.2 mg, 0.009 mmol, 32%) (MW_{Calc}+H=696.34; MW_{Obs}=696.4).

A-171 was prepared in a similar fashion to A-109 starting with compound 64 (16 mg, 0.028 mmol), commercially available 6-hydroxynicotinic acid (5.8 mg, 0.042 mmol) along with HOBT (2.1 mg, 0.014 mmol), triethylamine (9.7 uL, 0.070 mmol), and EDC-HCl (8.8 mg, 0.046 mmol) in dimethylacetamide (1.5 mL) for the first step to provide after hydrolysis and purification A-171 (10 mg, 0.015 mmol, 51%) (MW_{Calc}+H=682.33; MW_{Obs}=682.4).

A-172 was prepared in a similar fashion to A-109 starting with compound 64 (16 mg, 0.028 mmol), commercially available 5-bromo-2-fluorobenzoic acid (9.2 mg, 0.042 mmol) along with HOBT (2.1 mg, 0.014 mmol), triethylamine (9.7 uL, 0.070 mmol), and EDC-HCl (8.8 mg, 0.046 mmol) in dimethylacetamide (1.5 mL) for the first step to provide after hydrolysis and purification A-172 (7.4 mg, 0.010 mmol, 38%) (MW_{Calc}+H=763.23; MW_{Obs}=763.3).

A-173 was prepared in a similar fashion to A-109 starting with compound 64 (19 mg, 0.033 mmol), commercially available 1H-pyrazole-4-carboxylic acid (5.6 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-173 (10.2 mg, 0.016 mmol, 47%) (MW_{Calc}+H=655.33; MW_{Obs}=655.5).

A-174 was prepared in a similar fashion to A-109 starting with compound 64 (19 mg, 0.033 mmol), commercially available 1-methyl-3-(trifluoromethyl)-1H-pyrazole-4-carboxylic acid (9.6 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-174 (6.9 mg, 0.009 mmol, 27%) (MW_{Calc}+H=737.33; MW_{Obs}=737.4).

A-175 was prepared in a similar fashion to A-109 starting with compound 64 (19 mg, 0.033 mmol), commercially available 3-(trifluoromethyl)-1H-pyrazole-4-carboxylic acid (8.9 mg, 0.05 mmol) along with HOBT (2.8 mg, 0.017 mmol), 1 M triethylamine in THF (83 uL, 0.083 mmol), and EDC-HCl (10.5 mg, 0.055 mmol) in dimethylacetamide (1.5 mL) for the first step to provide after hydrolysis and purification A-175 (10.2 mg, 0.014 mmol, 43%) (MW_{Calc}+H=723.31; MW_{Obs}=723.4).

A-176 was prepared in a similar fashion to A-109 starting with compound 64 (16.0 mg, 0.028 mmol), commercially available (E)-3-(1H-pyrazol-4-yl)acrylic acid (6.8 mg, 0.042 mmol) along with HOBT (2.1 mg, 0.014 mmol), 1 M triethylamine in THF (70 uL, 0.070 mmol), and EDC-HCl (8.8 mg, 0.047 mmol) in dimethylacetamide (1.0 mL) for the first step to provide after hydrolysis and purification A-176 (8.5 mg, 0.012 mmol, 43%) (MW_{Calc}+H=681.34; MW_{Obs}=681.4).

A-177 was prepared in a similar fashion to A-109 starting with compound 64 (12 mg, 0.021 mmol), commercially available 1-methyl-1H-pyrazole-4-carboxylic acid (5.3 mg, 0.042 mmol) along with HOBT (3.2 mg, 0.021 mmol), triethylamine (15 uL, 0.105 mmol), and EDC-HCl (8 mg, 0.042 mmol) in dimethylacetamide (0.5 mL) for the first step to provide after hydrolysis and purification A-177 (7.2 mg, 0.011 mmol, 51%) (MW_{Calc}+H=669.34; MW_{Obs}=669.5).

A-178 was prepared in a similar fashion to A-109 starting with compound 64 (12 mg, 0.021 mmol), commercially available 1H-benzo[d][1,2,3]triazole-5-carboxylic acid (4.4 mg, 0.021 mmol) along with HOBT (2.8 mg, 0.021 mmol), triethylamine (8.7 uL, 0.063 mmol), and EDC-HCl (8 mg, 0.042 mmol) in dimethylacetamide (0.8 mL) for the first step to provide after hydrolysis and purification A-178 (5.3 mg, 0.007 mmol, 33%) (MW_{Calc}+H=706.34; MW_{Obs}=706.6).

A-179 was prepared in a similar fashion to A-109 starting with compound 64 (12 mg, 0.021 mmol), commercially available 5-(2-methyl-1,3-thiazol-4-yl)-3-isoxazolecarboxylic acid (4.4 mg, 0.021 mmol) along with HOBT (2.8 mg, 0.021 mmol), triethylamine (8.7 uL, 0.063 mmol), and EDC-HCl (8 mg, 0.042 mmol) in dimethylacetamide (0.8 mL) for the first step to provide after hydrolysis and purification A-179 (15.7 mg, 0.021 mmol, 100%) (MW_{Calc}+H=753.31; MW_{Obs}=753.52).

### Via Acid Chloride Condensation

A-180 was prepared in a similar fashion to A-109 via an acid chloride condensation:

To a stirred solution of (S)-tert-butyl 6-(2-((2R,4S,5R,6R)-5-acetamido-6-((1R,2R)-3-azido-1,2-dihydroxypropyl)-4-hydroxy-2-(methoxycarbonyl)tetrahydro-2H-pyran-2-yl)ethyl)-9-oxa-2,6-diazaspiro[4.5]decane-2-carboxylate (63, 0.2 g, 0.333 mmol) in THF (3.00 mL) and water (0.240 mL) at 0° C. room temperature was added 1N trimethylphosphine (0.999 mL, 0.999 mmol). The reaction mixture was warmed to room temperature and stirred for 16 h, after which time the mixture was concentrated and azeotroped to dry with toluene (2×20 mL ea) to provide crude 64.

To a stirred solution of the concentrated amine (64) in DCM (3.21 mL) at room temperature was added triethylamine (0.464 mL, 3.33 mmol), and 3,4-dimethylbenzene-1-carbonyl chloride (0.084 g, 0.499 mmol). The reaction mixture was stirred for 2 h, after which time the reaction was quenched with saturated 1 N NaOH (4 mL), and then extracted with EtOAc (3×6 mL ea). The combined organic was concentrated, diluted with methanol (5 mL) and then K₂CO₃ (100 mg) followed by stirring for 2 h. The completely quenched reaction was diluted with water (5 mL), and then extracted with EtOAc (4×6 mL ea). The combined organic layers were concentrated, and purified over a Biotage Ultra SNAP column (25 g) eluting with a gradient of 30% to 100% EtOAc in heptane (10 CV) to give compound (S)-tert-butyl 6-(2-((2R,4S,5R,6R)-5-acetamido-6-((R,2R)-3-(3,4-dimethylbenzamido)-1,2-dihydroxypropyl)-4-hydroxy-2-(methoxycarbonyl)tetrahydro-2H-pyran-2-yl)ethyl)-9-oxa-2,6-diazaspiro[4.5]decane-2-carboxylate (0.22 g, 0.311 mmol, 93% yield) (MW_{Calc}+H=707.38; MW_{Obs}=707.81) after collection of the desired fractions, concentration, and drying under vacuum.

To a stirred solution of (S)-tert-butyl 6-(2-((2R,4S,5R,6R)-5-acetamido-6-((1R,2R)-3-(3,4-dimethylbenzamido)-1,2-dihydroxypropyl)-4-hydroxy-2-(methoxycarbonyl)tetrahydro-2H-pyran-2-yl)ethyl)-9-oxa-2,6-diazaspiro[4.5]decane-2-carboxylate (10 mg, 0.014 mmol) in MeOH (400 μL) and THF (400 μL) at room temperature was added 1 N aqueous NaOH (495 μL, 0.495 mmol). The reaction mixture was stirred at room temperature for 16 h, after which time the completed reaction was acidified with conc. HCl to pH 4, and purified over a reversed-phase C18 Xbridge HPLC column eluting with a water/acetonitrile gradient containing 0.1% NH₄OH to provide A-180 (6.0 mg, 0.009 mmol, 61%) (MW_{Calc}+H=715.37; MW_{Obs}=715.38) after collection of the desired fractions, concentration, and drying under vacuum.

A-181 was prepared in a similar fashion to A-180 starting with compound 64 (12 mg, 0.021 mmol), commercially available m-toluoylchloride (6.5 mg, 0.042 mmol) for the first step to provide after hydrolysis and purification A-181 (4 mg, 0.006 mmol, 52%) (MW_{Calc}+H=679.35; MW_{Obs}=679.42).

A-182 was prepared in a similar fashion to A-180 starting with compound 64 (12 mg, 0.021 mmol), commercially available 3-chlorobenzoyl chloride (11 mg, 0.063 mmol) for the first step to provide after hydrolysis and purification A-182 (6.1 mg, 0.009 mmol, 42%) (MW_{Calc}+H=699.30; MW_{Obs}=699.35).

A-183 was prepared in a similar fashion to A-180 starting with compound 64 (12 mg, 0.021 mmol), commercially available 3-dimethylaminobenzoyl chloride hydrochloride (13.8 mg, 0.063 mmol) for the first step to provide after hydrolysis and purification A-183 (7.2 mg, 0.010 mmol, 49%) (MW_{Calc}+H=708.38; MW_{Obs}=708.36).

A-184 was prepared in a similar fashion to A-180 starting with compound 64 (12 mg, 0.021 mmol), commercially available 4-acetamidobenzoyl chloride (8.1 mg, 0.042 mmol) for the first step to provide after hydrolysis and purification A-184 (4.0 mg, 0.006 mmol, 27%) (MW_{Calc}+H=722.36; MW_{Obs}=722.42).

A-185 was prepared in a similar fashion to A-180 starting with compound 64 (12 mg, 0.021 mmol), commercially available 3,4,5-trifluoro benzoyl chloride (8.1 mg, 0.042 mmol) for the first step to provide after hydrolysis and purification A-185 (4.0 mg, 0.006 mmol, 27%) (MW_{Calc}+H=719.31; MW_{Obs}=719.51).

A-186 was prepared in a similar fashion to A-180 starting with compound 64 (12 mg, 0.021 mmol), commercially available 3,4-dichlorobenzoyl chloride (8.8 mg, 0.042 mmol) for the first step to provide after hydrolysis and purification A-186 (4.0 mg, 0.006 mmol, 26%) (MW_{Calc}+H=733.35; MW_{Obs}=733.36).

A-187 was prepared in a similar fashion to A-180 starting with compound 64 (12 mg, 0.021 mmol), commercially available 3,4-dichlorobenzoyl chloride (6.2 mg, 0.042 mmol) for the first step to provide after hydrolysis and purification A-187 (4.0 mg, 0.006 mmol, 29%) (MW_{Calc}+H=665.34; MW_{Obs}=665.52).

A-188 was prepared in a similar fashion to A-180 starting with compound 64 (12 mg, 0.021 mmol), commercially available 5-methylisoxazole-3-carbonyl chloride (9.1 mg, 0.063 mmol) for the first step to provide after hydrolysis and purification A-188 (4.5 mg, 0.007 mmol, 32%) (MW_{Calc}+H=670.33; MW_{Obs}=670.49).

### Preparation of A-189

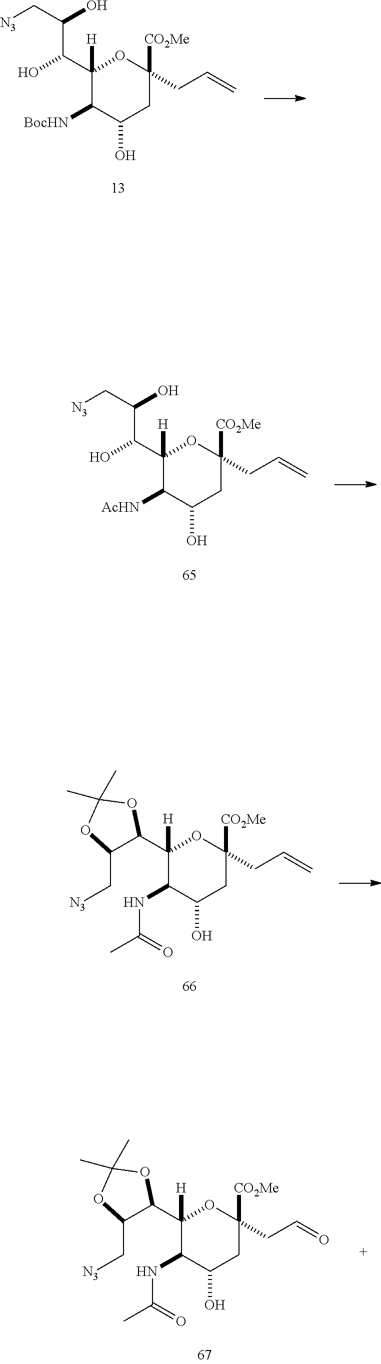

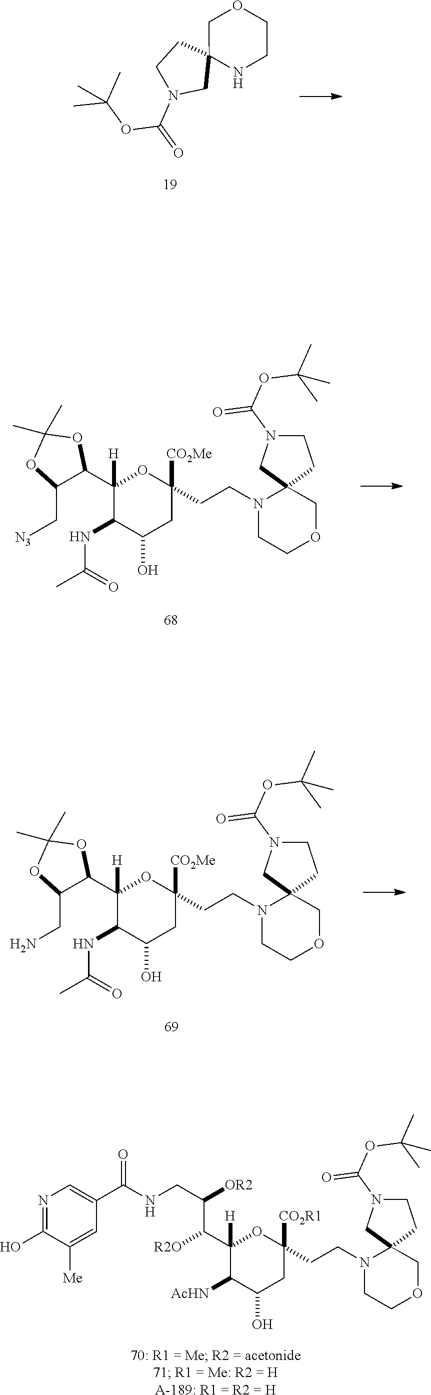

To a stirred solution of methyl (2R,4S,5R,6R)-2-allyl-6-((1R,2R)-3-azido-1,2-dihydroxypropyl)-5-((tert-butoxycarbonyl)amino)-4-hydroxytetrahydro-2H-pyran-2-carboxylate (13, 5 g, 11.62 mmol) was added 4 N HCl (14.52 ml, 58.08 mmol) in dioxane at room temperature followed by stirring for 16 h. The completed reaction was concentrated, and azeotroped to dry with toluene (3×30 mL ea). The crude amine residue was dissolved in THF (75 mL), and water (75 mL) at room temperature, followed by the addition of sodium bicarbonate (29.3 g, 348.47 mmol) and then a dropwise addition of acetic anhydride (5.48 ml, 58.08 mmol) over a 5-min period. The final reaction was stirred for 30 min, after which time it was extracted with EtOAc (3×150 mL ea), dried over Na2SO4, and concentrated to dryness. The crude residue was purified over a Biotage Ultra SNAP silica gel column (50 g) eluting with 5 CV 30 to 100% EtOAc in heptane, 5 CV 0 to 30% EtOAc in MeOH to provide compound 65 (3.9 g, 10.47 mmol, 90%) (MW_{Calc}+H=373.16; MW_{Obs}=373.18) after collection of the desired fractions, concentration, and evaporation to dryness under vacuum.

To a stirred solution of methyl (2R,4S,5R,6R)-5-acetamido-2-allyl-6-((1R,2R)-3-azido-1,2-dihydroxypropyl)-4-hydroxytetrahydro-2H-pyran-2-carboxylate (65, 710 mg, 1.91 mmol) in 2,2-dimethoxypropane (4.69 mL, 38.13 mmol) and acetone (5 mL) was at room temperature added p-toluenesulfonic acid monohydrate (36.3 mg, 0.191 mmol). The reaction mixture was stirred for 2 h, followed by the addition of triethylamine (1.33 mL, 9.53 mmol), concentrated, and then sat. NaHCO₃ (7 mL). The quenched reaction mixture was extracted with EtOAc (3×10 mL ea), washed with brine (5 mL), dried over Na₂SO₄, filtered and concentrated to dry. The residue was and purified over a Biotage Ultra SNAP silica gel column (25 g) eluting with 5 CV 1:1 EtOAc:Heptane, then 5 CV of a gradient of 0 to 10% MeOH in EtOAc) to give compound 66 (720 mg, 1.746 mmol, 92%) (MW_{Calc}+Na=435.20; MW_{Obs}=435.29) after collection of the desired fractions, concentration, and evaporation to dryness under vacuum.

To a stirred solution of methyl (2R,4S,5R,6R)-5-acetamido-2-allyl-6-((4R,5R)-5-(azidomethyl)-2,2-dimethyl-1,3-dioxolan-4-yl)-4-hydroxytetrahydro-2H-pyran-2-carboxylate (66, 0.7 g, 1.70 mmol) in 1,4-dioxane (12.60 mL) and water (4.20 mL) at room temperature was added 2,6-lutidine (0.198 ml, 1.697 mmol), osmium tetroxide (0.216 ml, 0.034 mmol), and sodium periodate (1.452 g, 6.789 mmol). The reaction mixture was stirred for 3 hours, after which time the completed reaction was partitioned between EtOAc (10 mL) and water (10 mL). The aqueous layer was separated, extracted with EtOAc (3×10 mL ea), and the combined organic layers were dried over Na₂SO₄, filtered, and concentrated. The residue was purified over a Biotage Ultra SNAP silica gel column (25 g) eluting with 3 CV with 20% EtOAc in heptane, 5 CV 50 to 100% EtOAc in heptane, and 3 CV EtOAc to provide compound 67 (0.58 g, 1.400 mmol, 82% yield) (MW_{Calc}+Na=437.18; MW_{Obs}=437.26) after collection of the desired fractions, concentration, and evaporation to dryness under vacuum.

To a stirred solution of methyl (2S,4S,5R,6R)-5-acetamido-6-((4R,5R)-5-(azidomethyl)-2,2-dimethyl-1,3-dioxolan-4-yl)-4-hydroxy-2-(2-oxoethyl)tetrahydro-2H-pyran-2-carboxylate (67, 580 mg, 1.40 mmol) in dichloroethane (13.2 mL) at room temperature was added acetic acid (561 μL, 9.797 mmol), (S)-tert-butyl 9-oxa-2,6-diazaspiro[4.5]decane-2-carboxylate (19, 339 mg, 1.40 mmol), and dried 4A molecular sieves (3 g, 2 g/mmol). The mixture was stirred for 2 h, after which time sodium triacetoxyborohydride (593 mg, 2.799 mmol)) was added with stirring for an additional 45 min. The completed reaction was diluted with EtOAc (15 mL) and filtered over a pad of Celite (10 g) eluting with EtOAc (2×10 mL ea). The filtrate was quenched with aq NaHCO₃ (10 mL), the layers separated, and the aqueous layer was extracted with EtOAc (3×20 mL ea). The combined organic layers were dried over Na₂SO₄, filtered and concentrated to dry. The residue was and purified over a Biotage Ultra SNAP silica gel column (25 g) eluting with 5 CV of 50 to 100% EtOAc in heptane and then 5 CV of 0 to 20% MeOH in EtOAc to provide compound 68 (800 mg, 1.249 mmol, 89% yield) (MW_{Calc}+Na=663.34; MW_{Obs}=663.36) after collection of the desired fractions, concentration, and evaporation to dryness under vacuum.

To a stirred solution of (S)-tert-butyl 6-(2-((2R,4S,5R,6R)-5-acetamido-6-((4R,5R)-5-(azidomethyl)-2,2-dimethyl-1,3-dioxolan-4-yl)-4-hydroxy-2-(methoxycarbonyl)tetrahydro-2H-pyran-2-yl)ethyl)-9-oxa-2,6-diazaspiro[4.5]decane-2-carboxylate (68, 600 mg, 0.936 mmol) in THF (10 mL) and water (0.5 mL) at room temperature was added 1.0 M trimethylphosphine in THF (3.0 mL, 3.00 mmol).

The mixture was stirred for 2 h, after which time the completed reaction mixture was concentrated, and then azeotroped to dry with acetonitrile (3×10 mL ea) to provide crude compound 69 (MW_{Calc}+H=615.35; MW_{Obs}=615.53) (assuming 100% conversion), which was used in the next step without further purification.

To a stirred solution of (S)-tert-butyl 6-(2-((2R,4S,5R,6R)-5-acetamido-6-((4R,5R)-5-(aminomethyl)-2,2-dimethyl-1,3-dioxolan-4-yl)-4-hydroxy-2-(methoxycarbonyl)tetrahydro-2H-pyran-2-yl)ethyl)-9-oxa-2,6-diazaspiro[4.5]decane-2-carboxylate (69, 32 mg, 0.052 mmol) in acetonitrile (0.5 mL) at room temperature was added 6-hydroxy-5-methylnicotinic acid (8.77 mg, 0.057 mmol), triethylamine (50 μL, 0.359 mmol), HOBT (1.594 mg, 0.010 mmol) and EDC (10.98 mg, 0.057 mmol). The reaction mixture was stirred at room temperature for 16 h, after which time the completed reaction mixture was quenched with water (5 mL) and extracted ethyl acetate (2×10 mL ea). The combined organic layers were over Na₂SO₄, filtered and concentrated to dry. The crude residue was purified to provide compound 70 (13.8 mg, 0.018 mmol, 35%) (MW_{Calc}+H=750.38; MW_{Obs}=750.62) after collection of the desired fractions, concentration, and evaporation to dryness under vacuum.

To a stirred solution of (S)-tert-butyl 6-(2-((2R,4S,5R,6R)-5-acetamido-4-hydroxy-6-((4R,5R)-5-((6-hydroxy-5-methylnicotinamido)methyl)-2,2-dimethyl-1,3-dioxolan-4-yl)-2-(methoxycarbonyl)tetrahydro-2H-pyran-2-yl)ethyl)-9-oxa-2,6-diazaspiro[4.5]decane-2-carboxylate (70, 13.8 mg, 0.018 mmol) in methanol (1.0 mL) and water (0.5 mL) at room temperature was added p-toluenesulfonic acid (1.9 mg, 0.010 mmol) for 24 h. The mostly completed reaction was cooled to room temperature, concentrated, and azeotroped to dryness with toluene (2×10 mL). The crude mixture was purified to provide compound 71 (6.9 mg, 0.010 mmol, 53%) (MW_{Calc}+H=710.35; MW_{Obs}=710.58) after collection of the desired fractions, concentration, and evaporation to dryness under vacuum.

To a stirred solution of (S)-tert-butyl 6-(2-((2R,4S,5R,6R)-5-acetamido-6-((1R,2R)-1,2-dihydroxy-3-(6-hydroxy-5-methylnicotinamido)propyl)-4-hydroxy-2-(methoxycarbonyl)tetrahydro-2H-pyran-2-yl)ethyl)-9-oxa-2,6-diazaspiro[4.5]decane-2-carboxylate (71, 6.0 mg, 8.453 μmol) in methanol (2.0 mL) at room temperature was added 2.0 M aqueous sodium hydroxide (0.5 mL). The mixture was stirred for 16 h, after which time the completed reaction was quenched with 1N aq HCl (1.0 mL), concentrated, and azeotroped to dry with toluene (5×5 mL ea). The crude mixture was purified to provide A-189 (0.9 mg, 0.001 mmol, 15%) (MW_{Calc}+H=696.34; MW_{Obs}=696.6) after collection of the desired fractions, concentration, and evaporation to dryness under vacuum.

### Preparation of A-190 to A-192

A-190 was prepared in a similar fashion to A-189 starting with compound 68 (20 mg, 0.031 mmol), commercially available 3,5-dimethylbenzoic acid (9.4 mg, 0.062 mmol) for the first step to provide after hydrolysis and purification A-190 (6.0 mg, 0.009 mmol, 29%) (MW_{Calc}+Na=715.36; MW_{Obs}=715.45).

A-191 was prepared in a similar fashion to A-189 starting with compound 69 (100 mg, 0.163 mmol), commercially available 1,5-dimethyl-6-oxo-1,6-dihydropyridine-3-carboxylic acid (37 mg, 0.221 mmol) for the first step to provide after hydrolysis and purification A-191 (2.2 mg, 0.003 mmol, 2%) (MW_{Calc}+H=710.36; MW_{Obs}=710.5).

A-192 was prepared in a similar fashion to A-189 starting with compound 69 (100 mg, 0.163 mmol), commercially available 4-fluoro-3,5-dimethylbenzoic acid (41 mg, 0.244 mmol) for the first step to provide after hydrolysis and purification A-192 (5.3 mg, 0.007 mmol, 5%) (MW_{Calc}+H=711.36; MW_{Obs}=711.5).

### Preparation of A-193

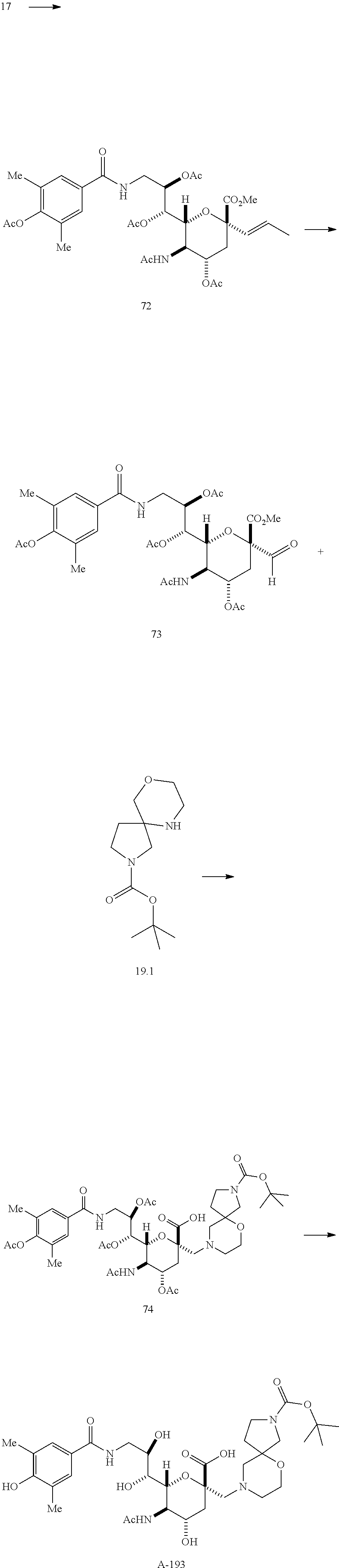

To a stirred solution of (1R,2R)-1-((2R,3R,4S,6R)-3-acetamido-4-acetoxy-6-allyl-6-(methoxycarbonyl)tetrahydro-2H-pyran-2-yl)-3-(4-acetoxy-3,5-dimethylbenzamido)propane-1,2-diyl diacetate (17, 250 mg, 0.377 mmol) in toluene (5.5 mL) was added trans-dichlorobis-(benzonitrilo)palladium (72.4 mg, 0.189 mmol), followed by degassing the suspension with nitrogen for 10 minutes. The suspension was heated to 90° C. for over 18 hours, after which time the reaction mixture was cooled to room temperature, filtered on a Celite pad and rinsed with EtOAc. [Note: An aliquot of the reaction was evaporated and monitored by 1H NMR for conversion (olefin doublet at 5.41 ppm in CDCl3) showing an approximate 15% conversions. The reaction mixture was cooled to ambient temperature, filtered on a Celite pad and rinsed with EtOAc.] The solvents were evaporated and the residue dissolved toluene (5 mL) followed by the addition of trans-dichlorobis-(benzonitrilo)palladium (72.4 mg, 0.189 mmol), and the reaction mixture was heated at 90° C. for 16 h. The partially completed reaction was cooled to room temperature, filtered through a pad of Celite (5 g) eluting with EtOAc. The filtrate was concentrated and purified over a Biotage SNAP silica gel column (10 g) eluting with 0-100% EtOAc in heptane to provide an inseparable 2:1 mixture of compound 17 to 72 (180 mg, 0.272 mmol, 72% yield), which was used in the next step without further purification. purification. (MW_{Calc}+H=663.27; MW_{Obs}=663.1).

To a stirred solution with (1R,2R)-1-((2R,3R,4S,6S)-3-acetamido-4-acetoxy-6-(methoxycarbonyl)-6-((E)-prop-1-en-1-yl)tetrahydro-2H-pyran-2-yl)-3-(4-acetoxy-3,5-dimethylbenzamido)propane-1,2-diyl diacetate (72, 14 mg, 0.021 mmol) in methanol (3 mL) and DCM (1 mL) at −78° C. was added ozone over a 25 min period after which time the reaction was rendered ozone free by bubbling N₂ into the reaction for 10 min at −78° C. Dimethyl sulfide (0.1 ml, 1.36 mmol) added and the completed reaction was warmed to room temperature followed by diluting with EtOAc (5 mL) and water (2 mL) The layers were separated, and the organic layer was dried over Na₂SO₄, filtered and concentrated to dry to provide crude compound 73 (MW_{Calc}+H=651.23; MW_{Obs}=651.43), which was used without further purification.

To a stirred solution of 73 in DCM (2 mL) was added tert-butyl 2,7-diazaspiro[4.5]decane-2-carboxylate (19.1, 10.16 mg, 0.042 mmol) and 4A MS (150 mg) followed by stirring at room temperature for 2 h. Sodium triacetoxyborohydride (8.96 mg, 0.042 mmol) was added to the resultant mixture followed by stirring for an additional 1 h. The completed reaction was slowly quenched with NaHCO₃ (3 mL), and then extract with EtOAc (3×2 mL ea). The combined organic layers were washed with brine (3 mL) and concentrated to dryness. The residue was purified by HPLC to provide compound 74 (3 mg, 3.43 μmol, 16% yield) (MW_{Calc}+H=875.40; MW_{Obs}=875.63).

To stirred solution of (1R,2R)-1-((2R,3R,4S,6S)-3-acetamido-4-acetoxy-6-((2-(tert-butoxycarbonyl)-2,7-diazaspiro[4.5]decan-7-yl)methyl)-6-(methoxycarbonyl)tetrahydro-2H-pyran-2-yl)-3-(4-acetoxy-3,5-dimethylbenzamido)propane-1,2-diyl diacetate (74, 9 mg, 10.286 μmol) in methanol (0.4 mL) was added 1 N aqueous sodium hydroxide (0.35 mL, 0.35 mmol) after which time the reaction mixture was stirred for 22 h. The completed reaction was directly purified over a HPLC column to provide compound A-193 (4 mg, 5.77 μmol, 56% yield) (MW_{Calc}+H=693.37; MW_{Obs}=693.39) as a mixture of diastereomers.

### Preparation of A-194

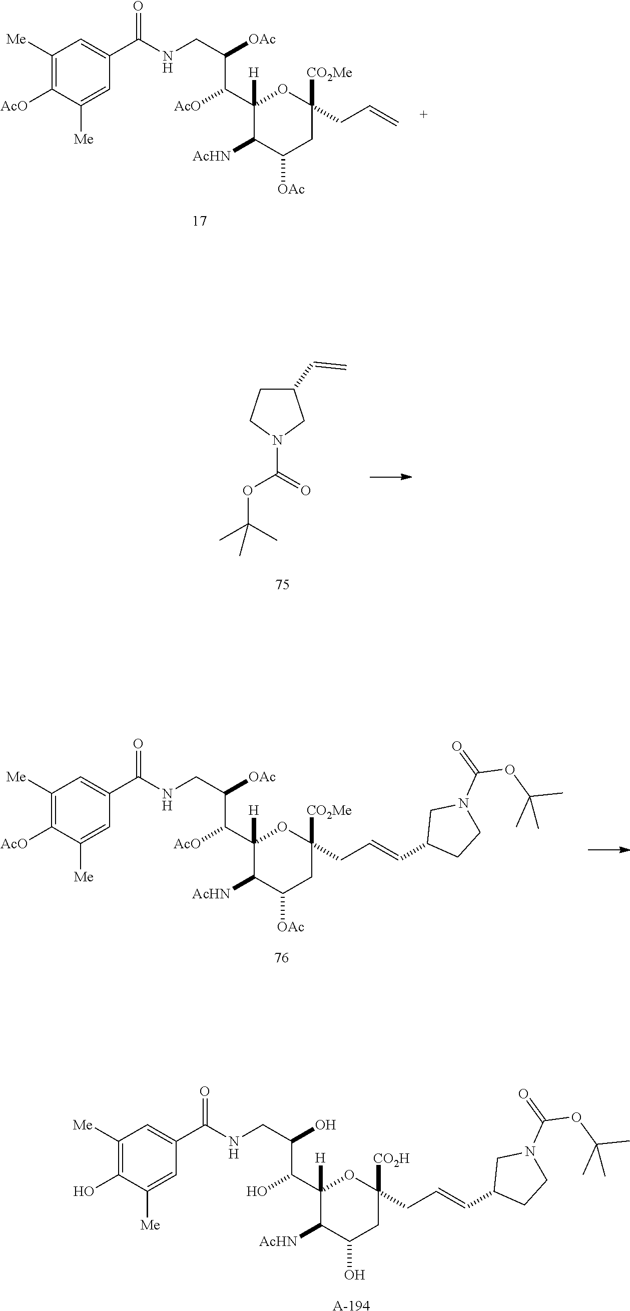

To stirred solution of (1R,2R)-1-((2R,3R,4S,6R)-3-acetamido-4-acetoxy-6-allyl-6-(methoxycarbonyl)tetrahydro-2H-pyran-2-yl)-3-(4-acetoxy-3,5-dimethylbenzamido)propane-1,2-diyl diacetate (17, 54 mg, 0.081 mmol) in dichloroethane (0.65 mL) at room temperature under a N₂ atmosphere was added commercially available tert-butyl (R)-3-vinylpyrrolidine-1-carboxylate (75, 48.2 mg, 0.244 mmol) followed by Hoveyda-Grubbs Catalyst 2nd Generation (5.12 mg, 8.149 μmol) and p-benzoquinone (3.52 mg, 0.033 mmol). The reaction was warmed to reflux and stirred for 16 h. The reaction was cooled to room temperature, and an additional Hoveyda-Grubbs Catalyst 2nd Generation (5.12 mg, 8.149 μmol) was added followed by warming to reflux and stirring for an additional 5 h. The completed reaction was cooled to room temperature, diluted with DMSO (0.1 mL) and stirred for 16 h.

After concentrating to half the volume, the resultant solution was purified directly over Biotage SNAP silica gel (10 g) eluting with 30-100% ethyl acetate in heptane (10 CV), and then with 0-30% ethyl acetate in methanol to give compound 76 (50.0 mg, 0.060 mmol, 74%) (MW_{Calc}+H=832.38; MW_{Obs}=832.47).

To a stirred solution of (1R,2R)-1-((2R,3R,4S,6R)-3-acetamido-4-acetoxy-6-((E)-3-((R)-1-(tert-butoxycarbonyl)pyrrolidin-3-yl)allyl)-6-(methoxycarbonyl)tetrahydro-2H-pyran-2-yl)-3-(4-acetoxy-3,5-dimethylbenzamido)propane-1,2-diyl diacetate (76, 50 mg, 0.06 mmol) in MeOH (1488 μl, 36.783 mmol) at 0° C. was added NaOH (1022 μl, 1.022 mmol). The mixture was stirred at room temperature for 16 h. The reaction mixture was directly submitted to analytical group for HPLC purification to provide compound A-194 (11.9 mg, 0.018 mmol, 31%) (MW_{Calc}+H=650.32; MW_{Obs}=650.5).

### Preparation of A-195 to A-199

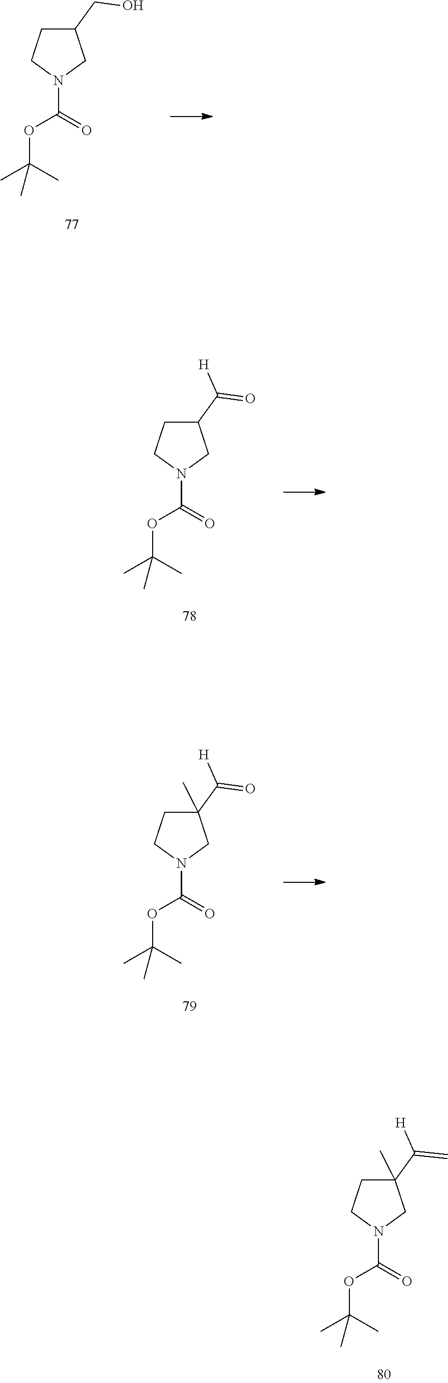

To a stirred solution of commercially available tert-butyl 3-(hydroxymethyl)pyrrolidine-1-carboxylate (77, 0.6 g, 2.981 mmol) in DCM (9.00 mL) at room temperature was added sodium bicarbonate (1.252 g, 14.906 mmol) and Dess-Martin periodinane (1.517 g, 3.577 mmol). The reaction mixture was stirred for 2 h after which time the mixture was quenched with sat. NaHCO₃ (6 mL) and Na₂S₂O₃ (6 mL) followed by being extracted with EtOAc (3×14 mL ea). The combined organic layers were washed with brine (1×10 mL), dried over Na₂SO₄, filtered and concentrated to provide compound 78 (0.50 g, 2.98 mmol, 84%), which was used in the next reaction without further purification.

To a stirred solution of tert-butyl 3-formylpyrrolidine-1-carboxylate (78, 0.5 g, 2.51 mmol) in THF (7.50 mL) was added 1 M tBuOK (5.02 mL, 5.019 mmol) in THF at 0° C. followed by stirring for 10 min. after which time methyl iodide (1.26 ml, 20.075 mmol). The reaction mixture was stirred at 0° C. for 2 h, and then quenched with sat. NaHCO₃ (10 mL). The resultant mixture was extracted with EtOAc (3×10 mL ea), and the combined organic layers were washed with brine (1×10 mL), dried over Na₂SO₄, filtered and concentrated to give compound 79 (0.50 g, 2.34 mmol, 93% yield).

To a stirred solution methyltriphenylphosphonium bromide (4.19 g, 11.72 mmol) in THF (15.00 mL) at 0° C. under a N₂ atmosphere was added 1 M LHMDS (9.38 ml, 9.38 mmol) in THF followed by stirring for 30 min. Tert-butyl 3-formyl-3-methylpyrrolidine-1-carboxylate (79, 0.5 g, 2.34 mmol) in THF (1 mL) was added at 0° C. followed by allowing the reaction to warm to room temperature and stirred for 16 h. The completed reaction was quenched with saturated NH₄Cl (4 mL) and extracted with EtOAc (2×5 mL ea). The combined organic layers were washed with brine (1×10 mL), dried over Na₂SO₄, filtered, concentrated, and then purified over a Biotage SNAP column (25 g) eluting with 0-50% ethyl acetate in heptane to give tert-butyl 3-methyl-3-vinylpyrrolidine-1-carboxylate 80 (0.30 g, 1.42 mmol, 61%) after collection, concentrating, and vacuum to dryness.

A-195 was prepared in a similar fashion to A-194 starting with compound 17 (0.30 g, 0.453 mmol) and tert-butyl 3-methyl-3-vinylpyrrolidine-1-carboxylate (80, 0.29 g, 1.36 mmol), to provide after purification A-195 (43 mg, 0.065 mmol, 14% overall yield) (MW_{Calc}+H=664.34; MW_{Obs}=664.20).

A-196 was prepared by dissolving the fully protected intermediate of A-195 (60.0 mg, 0.071 mmol) in ethyl acetate (1.2 mL) and methanol (0.9 mL) at room temperature followed by the addition of 10% palladium on carbon (75 mg) and then stirring the mixture under hydrogen gas at above atmospheric pressure for 16 h. The completed reaction is filtered over Celite (3 g) eluting with 10% methanol in ethyl acetate (20 mL). The filtrate was concentrated to a syrup. The syrup was dissolved in methanol (0.9 mL) and THF (0.9 mL), 1 N NaOH (0.71 mL, 0.71 mmol) was added, and the resultant mixture was stirred for 24 h, and the 35° C. for 24 h. The resultant completed reaction was purified directly via HPLC to provide A-196 (10.3 mg, 0.015 mmol, 22%) (MW_{Calc}+Na=688.36; MW_{Obs}=688.25).

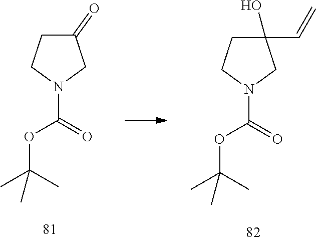

To a stirred solution of commercially available tert-butyl 3-oxopyrrolidine-1-carboxylate (81, 509 mg, 2.75 mmol) in Et₂O (8.57 mL) at −45° C. was added dropwise 1 M vinyl magnesium bromide (5.50 mL, 5.50 mmol) in Et₂O followed by allowing the reaction to warm to room temperature and stir for 16 h. The completed reaction was cooled to −45° C., and then quenched with sat. NH₄Cl (10 mL). The resultant mixture was warmed to room temperature, and the layers were separated. The aqueous layer was extracted with EtOAc (2×20 mL ea), and the combined organic layers were washed with NaHCO₃ (10 mL), brine (10 mL), dried over Na₂SO₄, filtered and concentrated. The crude oil was purified by HPLC to provide compound 82 (318 mg, 1.49 mmol, 54%).

A-197 was prepared in a similar fashion to A-194 starting with compound 17 (50 mg, 0.075 mmol) and tert-butyl 3-hydroxy-3-vinylpyrrolidine-1-carboxylate (82, 48.3 g, 0.226 mmol), to provide after purification A-197 (1.8 mg, 0.003 mmol, 4% overall yield) (MW_{Calc}+H=666.32; MW_{Obs}=666.5)

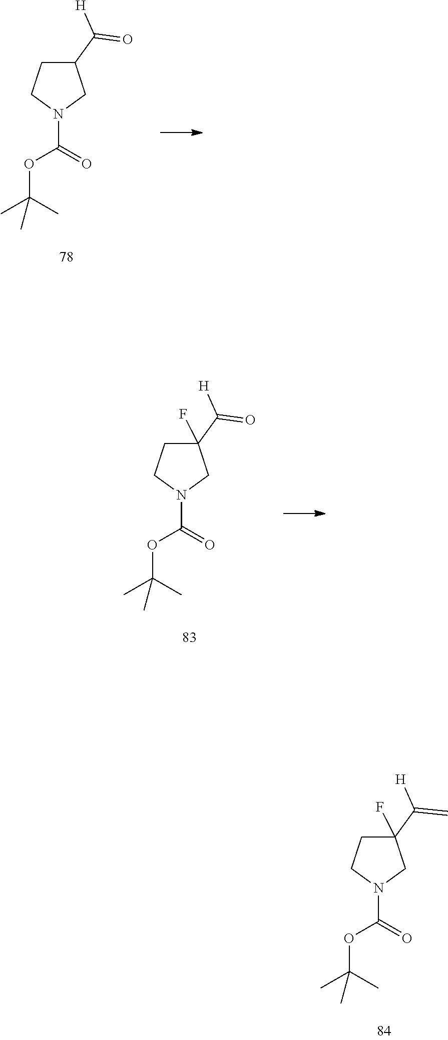

To a stirred solution of tert-butyl 3-formylpyrrolidine-1-carboxylate (78, 760 mg, 3.81 mmol) in THF (9.4 mL) was added pyrrolidine-3-carboxylic acid (329 mg, 2.86 mmol) and NFSI (4.21 g, 13.35 mmol) at room temperature. The reaction mixture was stirred for 16 h after which time the reaction was diluted with EtOAc (30 mL) and washed with water (20 mL). The aqueous layer was extracted with EtOAc (2×20 mL ea), and the combined organic layers were washed with brine (10 mL), dried over Na₂SO₄, filtered and concentrated. the crude oil was purified twice over a Biotage SNAP column (25 g) eluting with 1:1 ethyl acetate to heptane to provide compound 83 (520 mg, 2.39 mmol, 63%) after collection of the desired fractions, concentration and drying under vacuum.

To a stirred solution methyltriphenylphosphonium bromide (2.47 g, 6.91 mmol) in THF (8.83 mL) at 0° C. was added 1 M LHMDS (5.52 mL, 5.52 mmol) followed by stirring for 30 min after which time tert-butyl 3-fluoro-3-formylpyrrolidine-1-carboxylate (83, 300 mg, 1.38 mmol) in THF (1 mL) was added. The reaction mixture was allowed to warm up to room temperature and stirred for 16 h. The completed reaction was quenched with saturated NH₄Cl (4 mL) and extracted with EtOAc (2×5 mL ea). The combined organic layers were dried over Na₂SO₄, filtered and concentrated followed by purification over a Biotage SNAP column (25 g) eluting with a gradient of 0 to 50% ethyl acetate in heptane to provide compound 84 (26 mg, 0.121 mmol, 9%) after collection of the desired fractions, concentration and drying under vacuum.

A-198 was prepared in a similar fashion to A-194 starting with compound 17 (44 mg, 0.05 mmol) and tert-butyl 3-fluoro-3-vinylpyrrolidine-1-carboxylate (84, 42.9 g, 0.199 mmol), to provide after purification A-198 (3 mg, 0.0045 mmol, 19% overall yield) (MW_{Calc}+H=668.31; MW_{Obs}=667.6)

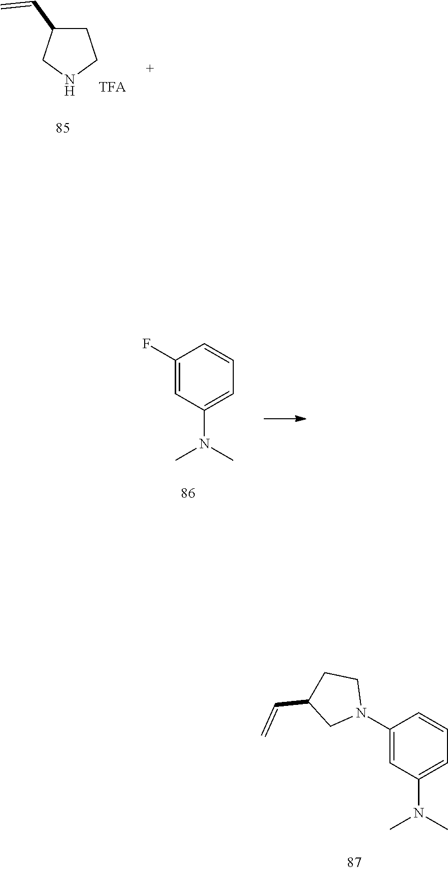

To a stirred solution of commercially available (R)-3-vinylpyrrolidine 2,2,2-trifluoroacetate (85, 200 mg, 0.947 mmol) in THF (2 mL) at room temperature was added commercially available 2-fluoro-N,N-dimethylpyridin-4-amine (86), 206 mg, 1.468 mmol) followed by triethylamine (0.396 mL, 2.841 mmol). The reaction mixture was heated in a microwave at 150 Watts for 5 h, after which time the mixture was cooled to room temperature, and applied directly to a Biotage SNAP Ultra silica gel column (10 g) eluting with 5 CV of 0 to 10% MeOH in DCM to provide (R)—N,N-dimethyl-2-(3-vinylpyrrolidin-1-yl)pyridin-4-amine (87, 189 mg, 0.870 mmol, 92% yield) (MW_{Calc}+H=217.16; MW_{Obs}=217.89) as a light brown solid after collection of the desired fractions, concentration and drying under vacuum.

A-199 was prepared in a similar fashion to A-194 starting with compound 17 (24 mg, 0.036 mmol) and (R)—N,N-dimethyl-2-(3-vinylpyrrolidin-1-yl)pyridin-4-amine (87, 39.4 mg, 0.181 mmol), to provide after hydrolysis and purification A-199 (9.37 mg, 0.014 mmol, 38%) (MW_{Calc}+H=669.34; MW_{Obs}=670.40).

### Preparation of A-200 and A-201

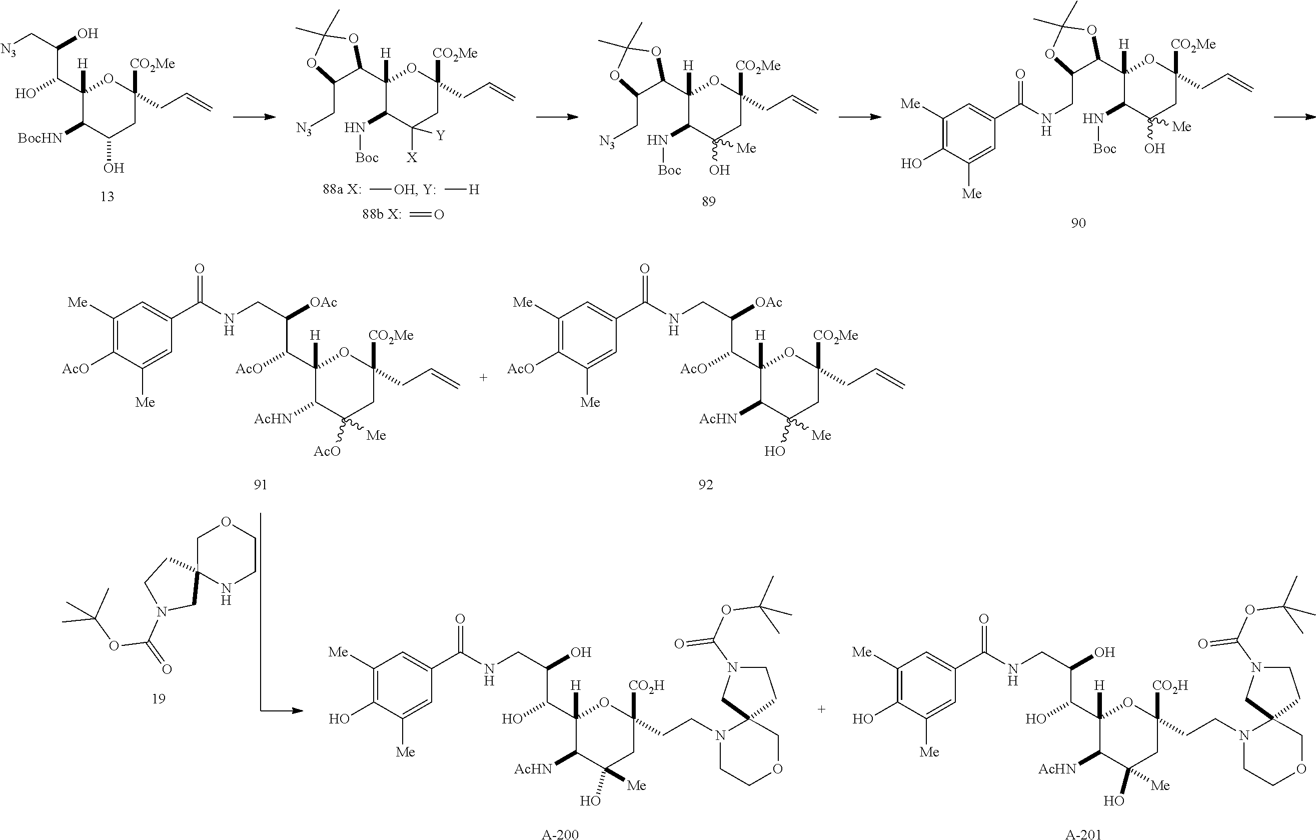

To a stirred solution of (2R,4S,5R,6R)-methyl 2-allyl-6-((1R,2R)-3-azido-1,2-dihydroxypropyl)-5-((tert-butoxycarbonyl)amino)-4-hydroxytetrahydro-2H-pyran-2-carboxylate (13, 0.20 g, 0.465 mmol) in acetone (5 mL) at room temperature was added 2,2-dimethoxypropane (1.143 mL, 9.293 mmol) followed by p-toluenesulfonic acid monohydrate (9.0 mg, 0.046 mmol). The reaction was stirred for 16 h, after which time it was quenched with sat. NaHCO₃ (5 mL), extracted with EtOAc (3×2 mL ea), and the combined organic layers were washed with brine (2 mL), dried over Na₂SO₄, filtered, concentrated and vacuumed to dryness to provide crude compound 88a (ca 219 mg, 0.465 mmol, 100%) (MW_{Calc}+Na=493.24; MW_{Obs}=493.21) without further purification.

To a stirred solution of crude methyl (2R,4S,5R,6R)-2-allyl-6-((4S,5S)-5-(azidomethyl)-2,2-dimethyl-1,3-dioxolan-4-yl)-5-((tert-butoxycarbonyl)amino)-4-hydroxytetrahydro-2H-pyran-2-carboxylate (88a, 0.4 g, 0.85 mmol) in DCM (4.80 mL) at room temperature was added sodium bicarbonate (0.357 g, 4.251 mmol) followed by Dess-Martin periodinane (0.541 g, 1.275 mmol). The reaction mixture was stirred for 2 h, after which time it was quenched with sat. Na₂S₂O₃ (3 mL) and sat. NaHCO₃ (3 mL). The layers were separated, and the aqueous layer was extracted with EtOAc (3×6 mL ea). The combined organic layers were dried over Na₂SO₄, filtered, and concentrated. The residue was purified over a Biotage SNAP silica gel column (25 g) eluting with a 10 CV gradient of 10 to 100% EtOAc in heptane to provide the 4-oxo analog of 88b (0.25 g, 0.534 mmol, 63%) (MW_{Calc}+Na=491.24; MW_{Obs}=491.22) after collection of the desired fractions, concentration and drying under vacuum.

A stirred solution of zirconium(IV) chloride (111 ml, 1.334 mmol) in THF (27.5 mL) was warmed to 60° C. for 20 min, after which time it was cooled to −55° C. followed by the dropwise addition of 1.6 M methyl lithium (3.34 mL, 5.336 mmol) in THF. The mixture was stirred at −55° C. for 10 min, after which time it was warmed to 0° C., and stirred for 30 min. The resultant slightly yellow solution was cooled to −78° C. after which time was slowly added methyl (2R,5S,6R)-2-allyl-6-((4S,5S)-5-(azidomethyl)-2,2-dimethyl-1,3-dioxolan-4-yl)-5-((tert-butoxycarbonyl)amino)-4-oxotetrahydro-2H-pyran-2-carboxylate (88b, 0.25 g, 0.534 mmol) in THF (5 mL) over a 5 min period. The final reaction mixture was stirred at −78° C. for 20 min, after which time it was quenched with a 1:1 mixture of water to saturated NH₄Cl (10 mL) and allowed to warm to room temperature. The resulting mixture was diluted with EtOAc (30 mL), the layers separated, and the aqueous layer was extracted with EtOAc (3×30 mL ea). The combined organic layers were dried over Na₂SO₄, filtered, and concentrated. The residue was purified over a Biotage SNAP silica gel column (25 g) eluting with a 10 CV gradient of 0 to 100% EtOAc in heptane to provide compound 89 (0.22 g, 0.454 mmol, 85%) (MW_{Calc}+Na=507.25; MW_{Obs}=507.23) after collection of the desired fractions, concentration and drying under vacuum.

To a stirred solution of methyl (2R,5S,6R)-2-allyl-6-((4R,5R)-5-(azidomethyl)-2,2-dimethyl-1,3-dioxolan-4-yl)-5-((tert-butoxycarbonyl)amino)-4-hydroxy-4-methyltetrahydro-2H-pyran-2-carboxylate (89, 0.22 g, 0.454 mmol) in THF (3.30 mL) and water (0.327 mL) at room temperature was added 1 M trimethylphosphine (1.362 ml, 1.362 mmol) in THF. The reaction mixture was stirred for 16 h, after which time it was concentrated, and azeotroped to dry with toluene (2×20 mL ea). The residue was dissolved in acetonitrile (3.30 mL) followed by the addition of 4-hydroxy-3,5-dimethylbenzoic acid (0.121 g, 0.726 mmol), HOBt (0.035 g, 0.227 mmol), EDC (0.131 g, 0.681 mmol), and then triethylamine (0.190 mL, 1.362 mmol). The final reaction was stirred at room temperature for 5 h, after which time it was quenched with 1:1 saturated NaHCO₃ in water (3 mL) and extracted with EtOAc (3×5 mL ea). The combined organic layers were dried over Na₂SO₄, filtered, and concentrated. The residue was purified over a Biotage SNAP silica gel column (10 g) eluting with a 10 CV gradient of 20 to 100% EtOAc in heptane to provide compound 90 (0.180 g, 0.297 mmol, 65%) (MW_{Calc}+Na=629.32; MW_{Obs}=629.30) after collection of the desired fractions, concentration and drying under vacuum.

To methyl (2R,5S,6R)-2-allyl-5-((tert-butoxycarbonyl)amino)-4-hydroxy-6-((4R,5R)-5-((4-hydroxy-3,5-dimethylbenzamido)methyl)-2,2-dimethyl-1,3-dioxolan-4-yl)-4-methyltetrahydro-2H-pyran-2-carboxylate (90, 0.180 g, 0.297 mmol) was added 4 N HCl in dioxane (0.742 mL, 2.967 mmol) at room temperature. The reaction mixture was stirred for 2 h, after which time it was concentrated to dry. The resultant residue was diluted with DCM (2.70 mL) at room temperature followed by the addition of triethylamine (0.827 mL, 5.934 mmol), DMAP (7.3 mg, 0.059 mmol), and then acetic anhydride (0.168 ml, 1.78 mmol). The resultant reaction mixture was stirred for 2 h, after which time additional DMAP (7.3 mg, 0.059 mmol) was added, and stirred for 48 h. The final reaction mixture was concentrated, and then purified over a Biotage SNAP silica gel column (10 g) eluting with a 10 CV gradient of 20 to 100% EtOAc in heptane followed by a 5 CV gradient of 0 to 20% EtOAc in MeOH to provide compound 91 (0.080 g, 0.228 mmol, 40%) (MW_{Calc}+Na=699.28; MW_{Obs}=699.20), and compound 92 (0.1 g, 0.158 mmol, 53%) (MW_{Calc}+Na=657.27; MW_{Obs}=657.21) after collection of the desired fractions, concentration and drying under vacuum.

To a stirred solution of (1R,2R)-1-((2R,3S,6R)-3-acetamido-4-acetoxy-6-allyl-6-(methoxycarbonyl)-4-methyltetrahydro-2H-pyran-2-yl)-3-(4-acetoxy-3,5-dimethylbenzamido)propane-1,2-diyl diacetate (91, 80 mg, 0.118 mmol) in 1,4-dioxane (2.40 mL) and water (0.48 mL) at room temperature was added 2,6-lutidine (27.5 μl, 0.236 mmol), osmium tetroxide (15.03 μl, 2.364 μmol), and sodium periodate (101 mg, 0.473 mmol). The reaction was stirred for 3 h, after which time the reaction was diluted with EtOAc (3 mL) and water (2 mL). The layers were separated, and the aqueous layer was extracted with EtOAc (3×4 mL ea). The combined organic layers were dried over Na₂SO₄, filtered, and concentrated. The crude aldehyde intermediate was dissolve in dichloroethane (1.20 mL) at room temperature followed by the addition of acetic acid (47.4 μl, 0.828 mmol) and tert-butyl (S)-9-oxa-2,6-diazaspiro[4.5]decane-2-carboxylate (19, 28.6 mg, 0.118 mmol), and 4A molecular sieves (2 g/mmol). The suspension was stirred for 2 h, after which time sodium triacetoxyborohydride (50.1 mg, 0.236 mmol) was added followed by stirring for 24 h. The reaction was quenched with sat. NaHCO₃ (2 mL), extracted with EtOAc (3×3 mL ea), dried over Na₂SO₄, filtered, and concentrated. The protected intermediate was purified over a Biotage SNAP silica gel column (10 g) eluting with a 10 CV gradient of 30 to 100% EtOAc in heptane to provide the protected intermediate. The protected intermediate was dissolved in MeOH (1.20 mL) at room temperature followed by the addition of 1 M aqueous NaOH (1.182 mL, 1.182 mmol). The final reaction mixture was stirred for 24 h, after which time it was neutralized with 4 N acetic acid in water (0.3 mL, 1.20 mmol), and submitted directly to HPLC purification to provide A-200 (1.5 mg, 0.002 mmol, 1.8%) (MW_{Calc}+H=723.38; MW_{Obs}=723.50) and A-201 (3.1 mg, 0.0043 mmol, 3.6%) (MW_{Calc}+H=723.38; MW_{Obs}=723.40) after collection of the desired fractions, concentration and drying under vacuum.

### Preparation of A-202

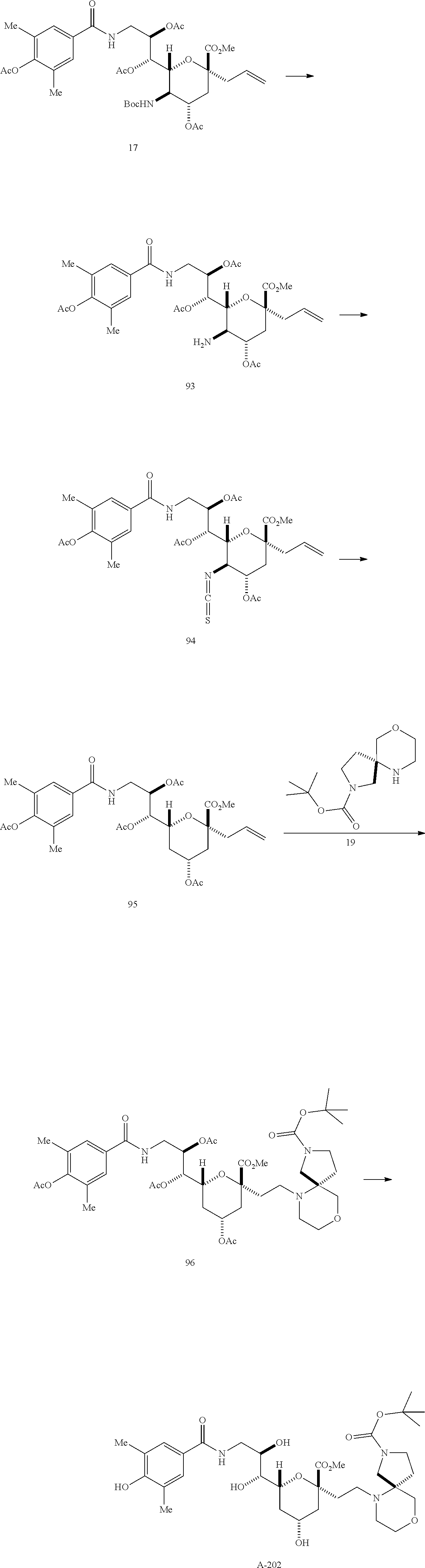

To a stirred solution of (1R,2R)-3-(4-acetoxy-3,5-dimethylbenzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (17, 100 mg, 0.139 mmol) in DCM (2.00 mL) at room temperature was slowly added TFA (1.00 mL). The reaction was stirred for 1 h, after which time it was azeotroped to dryness with toluene (3×5 mL ea). The resultant product, 93, was used in the next reaction without further purification as the TFA salt. (MW_{Calc}+H=621.26; MW_{Obs}=621.37).

To a stirred solution of (1R,2R)-3-(4-acetoxy-3,5-dimethylbenzamido)-1-((2R,3R,4S,6R)-4-acetoxy-6-allyl-3-amino-6-(methoxycarbonyl)tetrahydro-2H-pyran-2-yl)propane-1,2-diyl diacetate 2,2,2-trifluoroacetate (93, ca 50 mg, 0.068 mmol) in acetonitrile (0.50 mL) and water (1.00 mL) at room temperature was added sodium bicarbonate (51.5 mg, 0.613 mmol) followed by a dropwise addition of a solution of O-phenyl carbonochloridothioate (19.97 mg, 0.116 mmol) in acetonitrile (0.50 mL). The reaction mixture was stirred at room temperature 4 d, after which time the completed reaction was concentrated, and the resultant residue was diluted with sat. NaHCO₃ (20 mL) and EtOAc (20 mL). The layers were separated, and the aqueous layer was extracted with EtOAc (20 mL). The combined organic layers were washed with brine (15 mL), dried over Na2SO4, filtered, and concentrated. The final residue was purified over a Biotage Ultra SNAP silica gel column (10 g) eluting with 2 CV 5% EtOAC in heptane, a 5 CV gradient of 5 to 20% EtOAc, 3 CV 20% EtOAC in heptane, a 5 CV gradient of 20 to 50% EtOAc in heptane, followed by 3 CV 50% EtOAC in heptane to provide compound 94 (28.5 mg, 0.042 mmol, 62%) (MW_{Calc}+H=663.21; MW_{Obs}=663.27) after collection of the desired fractions, concentration and drying under vacuum.

To a stirred solution of (1R,2R)-3-(4-acetoxy-3,5-dimethylbenzamido)-1-((2R,3R,4S,6R)-4-acetoxy-6-allyl-3-isothiocyanato-6-(methoxycarbonyl)tetrahydro-2H-pyran-2-yl)propane-1,2-diyl diacetate (94, 28 mg, 0.042 mmol) in toluene (1.12 mL) at room temperature was added 1,1,1,3,3,3-hexamethyl-2-(trimethylsilyl)trisilane (67.8 μl, 0.22 mmol) followed by AIBN (1.041 mg, 6.338 μmol). The reaction mixture was warmed to 90° C., stirred for 70 min, cooled to room temperature, and then stirred for 16 h. The resultant mixture was purified over a Biotage Ultra SNAP silica gel column (10 g) eluting with a 5 CV gradient of 10 to 50% EtOAc, and 5 CV 50% EtOAC in heptane to provide compound 95 (9.7 mg, 0.015 mmol, 35%) (MW_{Calc}+Na=628.25; MW_{Obs}=628.35) after collection of the desired fractions, concentration and drying under vacuum.

To a stirred solution of (1S,2R)-3-(4-acetoxy-3,5-dimethylbenzamido)-1-((2R,4R,6R)-4-acetoxy-6-allyl-6-(methoxycarbonyl)tetrahydro-2H-pyran-2-yl)propane-1,2-diyl diacetate (95, 8 mg, 0.013 mmol) in 1,4-dioxane (0.288 mL) and water (0.096 mL) at room temperature was added 2,6-lutidine (3.08 μL, 0.026 mmol), osmium tetroxide (1.68 μL, 0.264 μmol), and sodium periodate (11.3 mg, 0.053 mmol). The reaction mixture was stirred for 2 h, after which time the completed reaction was diluted with DCM (10 mL) and water (5 mL). The layers were separated, and the aqueous layer was extracted with DCM (10 mL). The combined organic layers were dried over Na₂SO₄, filtered, and concentrated. The crude aldehyde intermediate was used in the next reaction without further purification.

To a stirred solution of (1S,2R)-3-(4-acetoxy-3,5-dimethylbenzamido)-1-((2R,4R,6S)-4-acetoxy-6-(methoxycarbonyl)-6-(2-oxoethyl)tetrahydro-2H-pyran-2-yl)propane-1,2-diyl diacetate (8.5 mg, 0.014 mmol) and (S)-tert-butyl 9-oxa-2,6-diazaspiro[4.5]decane-2-carboxylate (19, 6.78 mg, 0.028 mmol) in DCE (0.50 mL) at room temperature was added acetic acid (8.0 μL, 0.14 mmol) followed by 4A molecular sieves (30 mg). The suspension was stirred for 2 hours, after which time sodium triacetoxyborohydride (8.89 mg, 0.042 mmol) was then added. The final reaction mixture was stirred for 16 h, after which time it was diluted with sat. NaHCO₃ (10 mL) and EtOAc (15 mL). The layers were separated, and the aqueous layers was extracted with EtOAc (15 mL). The combined organic layers were washed with sat. NaHCO₃ (5 mL) and then with brine (10 mL), dried over Na₂SO₄, filtered, and concentrated. The residue was purified over a Biotage Ultra SNAP silica gel column (10 g) eluting with a 10 CV gradient of 1 to 10% methanol in DCM to provide compound 96 (11.8 mg, 0.013 mmol, 94%) (MW_{Calc}+H=834.39; MW_{Obs}=834.58) after collection of the desired fractions, concentration and drying under vacuum.

To a stirred solution of (1S,2R)-3-(4-acetoxy-3,5-dimethylbenzamido)-1-((2R,4R,6R)-4-acetoxy-6-(2-((S)-2-(tert-butoxycarbonyl)-9-oxa-2,6-diazaspiro[4.5]decan-6-yl)ethyl)-6-(methoxycarbonyl)-tetrahydro-2H-pyran-2-yl)propane-1,2-diyl diacetate (96, 11.6 mg, 0.014 mmol) in methanol (0.464 mL) and THF (0.464 mL) at room temperature was added 1N aqueous sodium hydroxide (0.417 mL, 0.417 mmol). The reaction mixture was stirred for 2 d, after which time it was neutralized with 2N HCl and submitted for HPLC purification to provide A-202 (4.3 mg, 0.006 mmol, 47%) (MW_{Calc}+H=652.34; MW_{Obs}=652.33) after collection of the desired fractions, concentration and drying under vacuum.

### Preparation of A-203

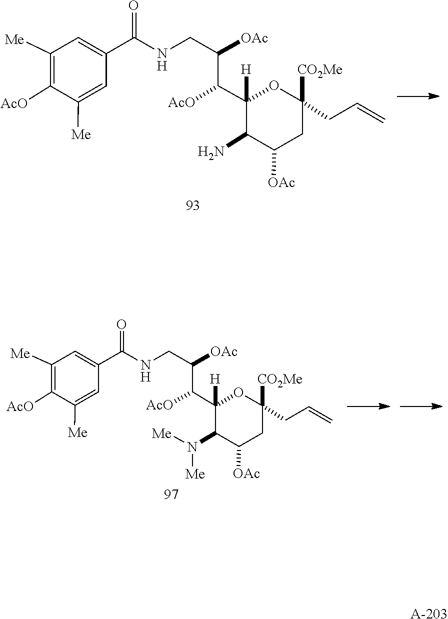 [SMILES: C=CC[C@]1(C(=O)OC)C[C@H](OC(C)=O)[C@@H](N)[C@H]([C@H](OC(C)=O)[C@@H](CNC(=O)c2cc(C)c(OC(C)=O)c(C)c2)OC(C)=O)O1 ; C=CC[C@]1(C(=O)OC)C[C@H](OC(C)=O)[C@@H](N(C)C)[C@H]([C@H](OC(C)=O)[C@@H](CNC(=O)c2cc(C)c(OC(C)=O)c(C)c2)OC(C)=O)O1 ; "A-203"]

To a stirred solution of (1R,2R)-3-(4-acetoxy-3,5-dimethylbenzamido)-1-((2R,3R,4S,6R)-4-acetoxy-6-allyl-3-amino-6-(methoxycarbonyl)tetrahydro-2H-pyran-2-yl)propane-1,2-diyl diacetate 2,2,2-trifluoroacetate (93, 78 mg, 0.106 mmol) in methanol (0.78 mL) at room temperature was added 37% formaldehyde in water (39.5 μL, 0.531 mmol) followed by sodium triacetoxyborohydride (113 mg, 0.531 mmol). The reaction mixture was stirred for 16 h, after which time it was concentrated, and diluted with sat. NaHCO₃ (10 mL) and EtOAc (15 mL). The layers were separated, and the aqueous layers was extracted with EtOAc (15 mL). The combined organic layers were washed with sat. NaHCO₃ (5 mL) and then with brine (10 mL), dried over Na₂SO₄, filtered, and concentrated. The residue was purified over a Biotage Ultra SNAP silica gel column (10 g) eluting with 3 CV of 1% MeOH in DCM, a 10 CV gradient of 1 to 8% methanol in DCM, then 3 CV of 8% MeOH in DCM to provide compound 97 (47.2 mg, 0.073 mmol, 69%) (MW_{Calc}+H=649.29; MW_{Obs}=649.46, after collection of the desired fractions, concentration and drying under vacuum.

A-203 was prepared in a similar fashion to A-202 starting with (1R,2R)-3-(4-acetoxy-3,5-dimethylbenzamido)-1-((2R,3R,4S,6S)-4-acetoxy-3-(dimethylamino)-6-(methoxycarbonyl)-6-(2-oxoethyl)tetrahydro-2H-pyran-2-yl)propane-1,2-diyl diacetate (97, 47 mg, 0.072 mmol) and (S)-tert-butyl 9-oxa-2,6-diazaspiro[4.5]decane-2-carboxylate (19, 22.8 mg, 0.094 mmol), to provide after hydrolysis and purification A-203 (15.0 mg, 0.022 mmol, 31%) (MW_{Calc}+H=695.45: MW_{Obs}=695.49) after collection of the desired fractions, concentration and drying under vacuum.

### Preparation of A-204 and A-205

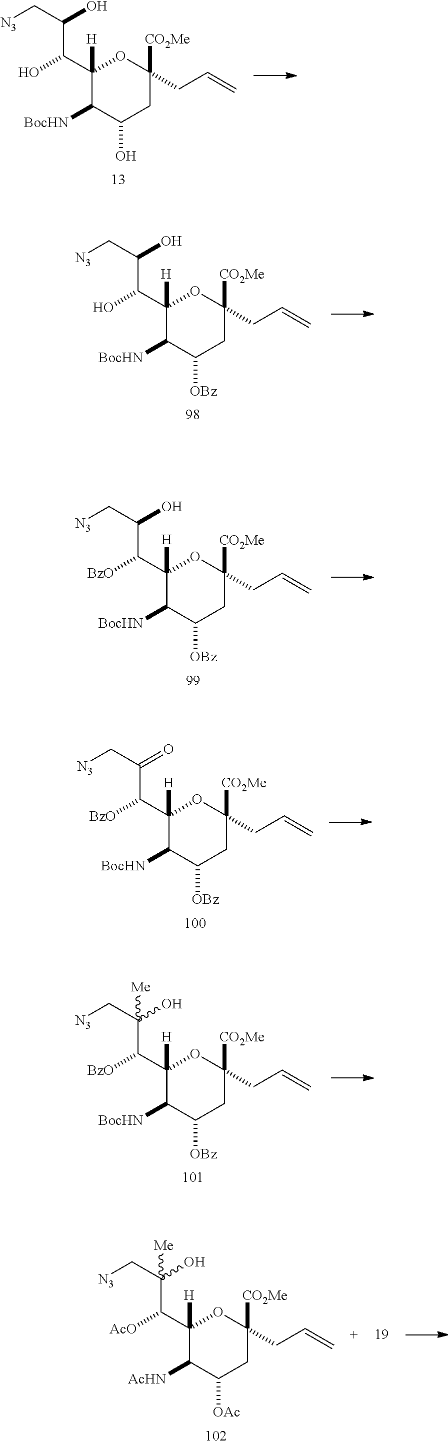

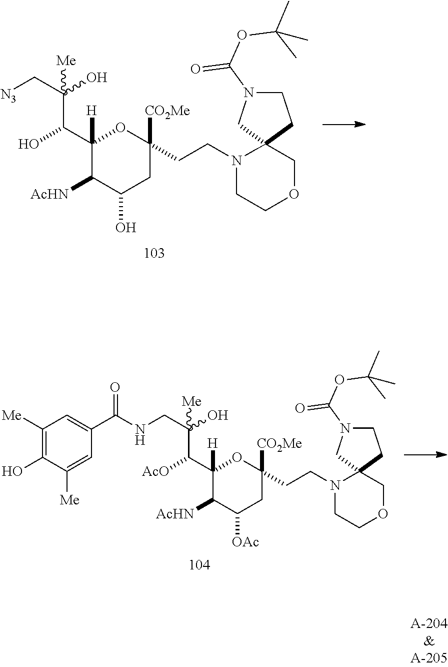

To a stirred solution of methyl (2R,4S,5R,6R)-2-allyl-6-((1S,2S)-3-azido-1,2-dihydroxypropyl)-5-((tert-butoxycarbonyl)amino)-4-hydroxytetrahydro-2H-pyran-2-carboxylate (13, 2.8 g, 6.505 mmol) in DCE (42.0 mL) at 0° C. was added 2,4,6-trimethylpyridine (8.60 mL, 65.047 mmol) and benzoyl chloride (1.888 mL, 16.262 mmol). The reaction mixture was allowed to slowly warm to room temperature, stirred for 16 h, after which time the mixture was cooled to 0° C., followed by adding benzoyl chloride (0.906 ml, 7.806 mmol). The resultant mixture was warmed to room temperature and stirred for 24 h. The final reaction mixture was slowly quenched with saturated NaHCO₃ (30 mL), the layers separated, and the aqueous layer was extracted with EtOAc (4×40 mL ea). The combined organic layers were dried over Na₂SO₄, filtered, and concentrated. The residue was purified over a Biotage Ultra SNAP silica gel column (50 g) eluting with a 10 CV gradient of 20 to 100% EtOAc in heptane to provide compound 98 (2 g, 3.74 mmol, 58%) (MW_{Calc}+Na=557.23; MW_{Obs}=557.35) and 99 (1 g, 1.566 mmol, 24%) (MW_{Calc}+Na=661.26; MW_{Obs}=661.4) after collection of the desired fractions, concentration and drying under vacuum.

To a stirred solution of methyl (2R,4S,5R,6R)-2-allyl-6-((1R,2R)-3-azido-1-(benzoyloxy)-2-hydroxypropyl)-4-(benzoyloxy)-5-((tert-butoxycarbonyl)amino)tetrahydro-2H-pyran-2-carboxylate (99, 700 mg, 1.096 mmol) in DCM (10.5 mL) at room temperature were added sodium bicarbonate (460 mg, 5.48 mmol) and Dess-Martin periodinane (558 mg, 1.315 mmol). The reaction mixture was stirred for 2 h, after which time it was quenched with saturated Na₂S₂O₃ (5 mL) and extracted with EtOAc (4×6 mL ea). The combined organic layers were dried over Na₂SO₄, filtered, and concentrated. The residue was purified over a Biotage Ultra SNAP silica gel column (10 g) eluting with a 10 CV gradient of 20 to 100% EtOAc in heptane to provide compound 100 (420 mg, 0.660 mmol, 60%) (MW_{Calc}+Na=659.24; MW_{Obs}=659.22) after collection of the desired fractions, concentration and drying under vacuum.

A stirred suspension of zirconium(IV) chloride (65.4 mL, 0.785 mmol) in THF (22.00 mL) at room temperature was warmed to 60° C. for 20 min, after which time the clear solution was cooled to −55° C. followed by the addition of methyllithium (1.963 ml, 3.141 mmol). The mixture was stirred for 10 min, warmed to 0° C., and stirred for 30 min. The slightly yellowish solution was cooled to −78° C., followed by the addition of a solution of methyl (2R,4S,5R,6R)-2-allyl-6-((R)-3-azido-1-(benzoyloxy)-2-oxopropyl)-4-(benzoyloxy)-5-((tert-butoxycarbonyl)amino)tetrahydro-2H-pyran-2-carboxylate (100, 0.2 g, 0.314 mmol) in THF (5 mL). The reaction mixture was stirred at −78° C. for 20 min, after which time it was quenched with 1:1 sat. NH₄Cl in water (25 mL) and warmed to room temperature. The mixture was diluted with EtOAc (30 mL), the layers separated, and the aqueous layer extracted with EtOAc (3×30 mL ea). The combined organic layers were dried over Na₂SO₄, filtered, and concentrated. The residue was purified over a Biotage Ultra SNAP silica gel column (25 g) eluting with a 10 CV gradient of 20 to 100% EtOAc in heptane to provide compound 101 (0.1 g, 0.153 mmol, 49%) (MW_{Calc}+Na=675.27; MW_{Obs}=675.31) after collection of the desired fractions, concentration and drying under vacuum.

To a stirred solution of methyl (2R,4S,5R,6R)-2-allyl-6-((1R)-3-azido-1-(benzoyloxy)-2-hydroxy-2-methylpropyl)-4-(benzoyloxy)-5-((tert-butoxycarbonyl)amino)tetrahydro-2H-pyran-2-carboxylate (101, 0.1 g, 0.153 mmol) in MeOH (2.00 mL) at room temperature was added K₂CO₃ (0.212 g, 1.532 mmol). The reaction mixture was stirred for 3 h, after which time it was diluted with sat. NaHCO₃ (3 mL), and extracted with EtOAc (3×5 mL ea). The combined organic layers were dried over Na₂SO₄, filtered, and concentrated. The residue was purified over a Biotage Ultra SNAP silica gel column (10 g) eluting with a 5 CV gradient of 30 to 100% EtOAc in heptane, then a 5 CV gradient of 0 to 30% MeOH to provide the debenzoyl intermediate (0.1 g, 0.153 mmol, 49%) (MW_{Calc}+Na=467.22; MW_{Obs}=467.20) after collection of the desired fractions, concentration and drying under vacuum. To methyl (2R,4S,5R,6R)-2-allyl-6-((1S)-3-azido-1,2-dihydroxy-2-methylpropyl)-5-((tert-butoxycarbonyl)amino)-4-hydroxytetrahydro-2H-pyran-2-carboxylate (60 mg, 0.135 mmol) at room temperature was added a solution of 4 N HCl in dioxane (337 μl, 1.35 mmol). The reaction mixture was stirred for 2 h, after which time it was concentrated, and azeotroped to dryness with toluene (2×10 mL ea). The resultant residue was dissolved with stirring in DCM (0.90 mL) followed by the addition of Et3N (376 μL, 2.70 mmol), DMAP (3.30 mg, 0.027 mmol) and Ac₂O (76 μL, 0.81 mmol). The final reaction mixture was stirred at room temperature for 48 h, after which time it was concentrated. The final residue was purified over a Biotage Ultra SNAP silica gel column (10 g) eluting with a 10 CV gradient of 0 to 20% MeOH to provide compound 102 (60 mg, 0.128 mmol, 94%) (MW_{Calc}+Na=493.20; MW_{Obs}=493.15) after collection of the desired fractions, concentration and drying under vacuum.

To a stirred solution of methyl (2R,4S,5R,6R)-5-acetamido-4-acetoxy-6-((1S)-1-acetoxy-3-azido-2-hydroxy-2-methylpropyl)-2-allyltetrahydro-2H-pyran-2-carboxylate (102, 60 mg, 0.128 mmol) in 1,4-dioxane (1.80 mL) and water (0.36 mL) at room temperature was added 2,6-lutidine (29.7 μL, 0.255 mmol), osmium tetroxide (16.21 μL, 2.551 μmol), and sodium periodate (109 mg, 0.51 mmol). The mixture was stirred for 3 h, after which time reaction was dilute with EtOAc (3 mL) and water (2 mL). The layers were separated, the aqueous layer was extracted with EtOAc (3×4 mL), and the combined organic layers were dried over Na₂SO₄, filtered, and concentrated to dryness. The resultant crude aldehyde was dissolved with stirring in DCE (0.90 mL) at room temperature followed by the addition of acetic acid (51.1 μl, 0.893 mmol), tert-butyl (S)-9-oxa-2,6-diazaspiro[4.5]-decane-2-carboxylate (19, 30.9 mg, 0.128 mmol), and oven dried 4A molecular sieves (256 mg). The mixture was stirred for 2 h, after which time sodium triacetoxyborohydride (54.1 mg, 0.255 mmol) was added the mixture was stirred for 24 h. The completed reaction was quenched with sat. NaHCO₃ (2 mL) and extracted with EtOAc (3×3 mL ea). The combined organic layers were dried over Na₂SO₄, filtered, and concentrated. The residue was purified over a Biotage Ultra SNAP silica gel column (10 g) eluting with a 5 CV gradient of 30 to 100% EtOAc in heptane, then a 5 CV gradient of 0 to 30% MeOH to provide tert-butyl (5R)-6-(2-((2R,4S,5R,6R)-5-acetamido-4-acetoxy-6-((1S)-1-acetoxy-3-azido-2-hydroxy-2-methylpropyl)-2-(methoxycarbonyl)tetrahydro-2H-pyran-2-yl)ethyl)-9-oxa-2,6-diazaspiro[4.5]decane-2-carboxylate (70 mg, 0.100 mmol, 79%) after collection of the desired fractions, concentration and drying under vacuum. The semi-pure product was dissolved in MeOH (1.20 mL) at room temperature followed by the addition of K₂CO₃ (176 mg, 1.275 mmol). The reaction mixture was stirred for 16 h, after which time it was diluted with sat. NaHCO₃ (2 mL) and EtOAc (5 mL). The layers were separated, and the aqueous layer was extracted with EtOAc (3×4 mL ea). The combined organic layers were dried over Na₂SO₄, filtered, and concentrated. The residue was purified over a Biotage Ultra SNAP silica gel column (10 g) eluting with a 5 CV gradient of 30 to 100% EtOAc in heptane, then a 5 CV gradient of 0 to 30% MeOH to provide compound 103 (20 mg, 0.033 mmol, 26%) (MW_{Calc}+Na=637.33; MW_{Obs}=633.21) after collection of the desired fractions, concentration and drying under vacuum.

To a stirred solution of tert-butyl (5S)-6-(2-((2R,4S,5R,6R)-5-acetamido-6-((1S)-3-azido-1,2-dihydroxy-2-methylpropyl)-4-hydroxy-2-(methoxycarbonyl)tetrahydro-2H-pyran-2-yl)ethyl)-9-oxa-2,6-diazaspiro[4.5]decane-2-carboxylate (103, 20 mg, 0.033 mmol) in THF (0.300 mL) and water (0.23 mL) at room temperature was added 1N trimethylphosphine (98 μL, 0.098 mmol). The reaction mixture was stirred for 16 h, after which time the completed intermediate was concentrated, concentrated and azeotroped to dry with toluene (2×10 mL ea). The resultant amine intermediate was dissolved with stirring in dimethylacetamide (0.40 mL) at room temperature followed by the addition of 4-hydroxy-3,5-dimethylbenzoic acid (10.81 mg, 0.065 mmol), HOBt (4.98 mg, 0.033 mmol), EDC (12.47 mg, 0.065 mmol), and finally triethylamine (22.68 μl, 0.163 mmol). The reaction mixture was stirred for 5 h providing crude 104, after which time 1 M NaOH (325 μL, 0.325 mmol) was added the final mixture was stirred for 1 d. The completed reaction was neutralized with 1 N HCl (325 μL, 0.325 mmol), filtered, eluted the filter pad with methanol (2×2 mL) ea, and submitted for HPLC purification to provide A-204 (0.5 mg, 0.0007 mmol, 2%) (MW_{Calc}+H=723.38; MW_{Obs}=723.33) and A-205 (0.5 mg, 0.0007 mmol, 2%) (MW_{Calc}+H=723.38; MW_{Obs}=723.28) after collection of the desired fractions, concentration and drying under vacuum.

### Preparation of A-206, A-207, and A-208

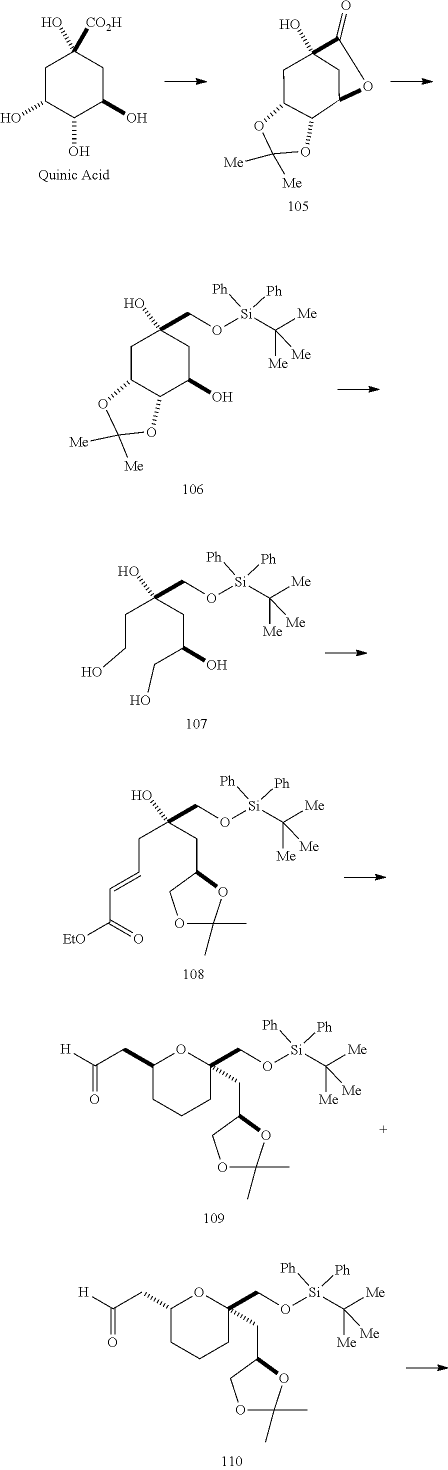

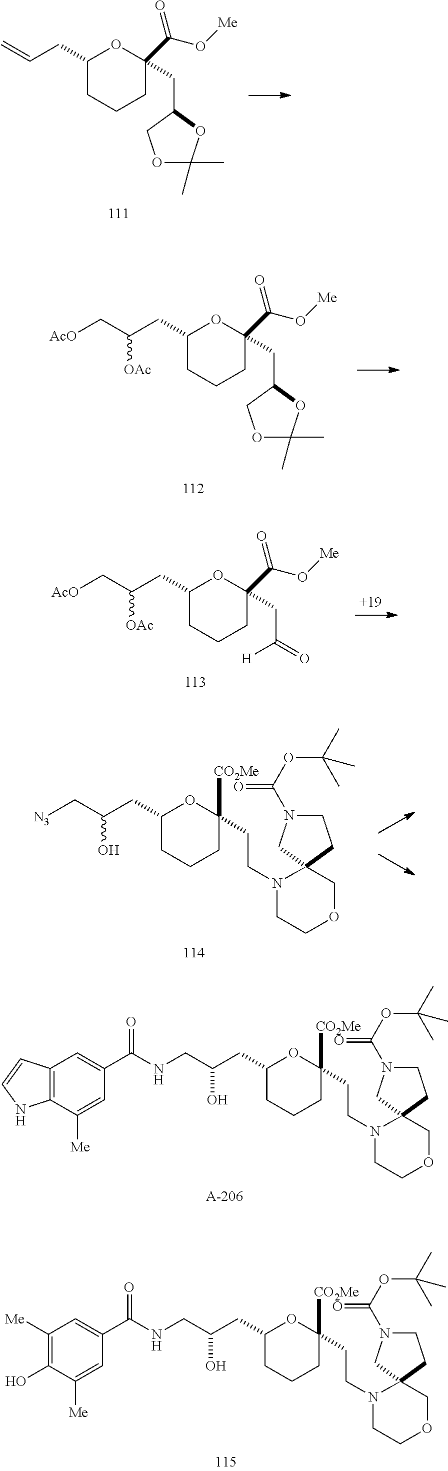

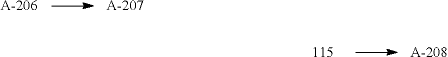

To a stirred suspension of quinic acid or (1S,3R,4S,5R)-1,3,4,5-tetrahydroxycyclohexanecarboxylic acid (33.2 g, 172.767 mmol) in toluene (500 mL) at room temperature was added 4-methylbenzenesulfonic acid (0.298 g, 1.728 mmol) followed by 2,2-dimethoxypropane (25 mL, 227.562 mmol). The mixture was stirred for 2 h, after which time it was warmed to reflux using Dean-Stark apparatus for the removal of water (undesired byproduct) from reaction mixture. Upon removal of water (approx. 200 mL), the mixture was cooled to room temperature and concentrated to a thick suspension. The solids were slurried in heptane (1 L), filtered, the filter pad washed with heptane (2×400 mL ea), and dried under vacuum to provide compound 105 (14.72 g, 68.7 mmol, 40%).

To a stirred solution of (3aR,4R,7S,8aR)-7-hydroxy-2,2-dimethyltetrahydro-4,7-methano[1,3]dioxolo-[4,5-c]oxepin-6(4H)-one (105, 3.54 g, 16.525 mmol) in THF (48 mL) at 0° C. was added dropwise 1 M LiAlH4 in THF (25.4 mL, 25.4 mmol), after which time the reaction mixture was warmed to room temperature, and then warmed to refluxing temperature. The mixture was stirred for 20 h, after which time it was cooled to 0° C. followed by a slow addition of water (0.956 mL, 53.055 mmol), 15% aq. sodium hydroxide (0.963 ml, 3.59 mmol), and finally water (2.87 mL, 159.164 mmol). The quenched reaction was stirred for 30 min, after which time Celite (14 g) was added and stirred for an additional 2 h. The suspension was filtered over a pad of Celite (10 g), the filter pad rinsed with MeOH (3×20 mL), and the filtrate concentrated. The residue was triturated with MeCN (20 mL) at 60° C., concentrated, and azeotroped to dry with MeCN (20 mL). The resulting product was used in the next step without further purification.

To a stirred solution of crude (3aS,4R,6R,7aR)-6-(hydroxymethyl)-2,2-dimethylhexahydrobenzo-[d][1,3]dioxole-4,6-diol (2.21 g, 10.126 mmol) in DMF (37 mL) at 0° C. was added imidazole (2.76 g, 40.504 mmol), and TBDPS-C1 (2.86 ml, 11.139 mmol). The reaction mixture was stirred at 0° C. for 16 h, after which time it was slowly warmed to 15° C. and stirred for 24 h. The reaction was quenched with 1:1 ratio of water:MTBE and stirred at room temperature for 16 h. The layers were separated, and the aqueous layer was extracted with MTBE (30 mL). The combined organic layers were washed with sat. brine (10 mL), dried over Na₂SO₄, filtered and concentrated to dry. The residue was purified over a Biotage Ultra SNAP silica gel column (50 g) eluting with 1 CV heptane, a 2 CV gradient of 0 to 5% EtOAc in heptane, 4 CV of 5% EtOAc in heptane, a 1 CV gradient of 5 to 20% EtOAc in heptane, a 3 CV gradient of 20 to 100% EtOAc in heptane, then 1 CV EtOAc to provide compound 106 (3.13 g, 6.85 mmol, 68%) (MW_{Calc}+Na=479.23; MW_{Obs}=479.38) after collection of the desired fractions, concentration and drying under vacuum.

To a stirred solution of (3aS,4R,6R,7aR)-6-(((tert-butyldiphenylsilyl)oxy)methyl)-2,2-dimethyl-hexahydrobenzo[d][1,3]dioxole-4,6-diol (106, 15.83 g, 34.665 mmol) in DCM (139 mL) at 0° C., was added pyridine (8.41 mL, 103.996 mmol) followed by a dropwise addition of benzoyl chloride (4.43 mL, 38.132 mmol). The reaction was maintained below 4° C. for 1 h, after which time additional benzoyl chloride (0.604 mL, 5.20 mmol), followed by stirring at 0° C. for 1 h. Benzoyl chloride (1.207 mL, 10.40 mmol) was added to the incomplete reaction, and the mixture was stirred at 0° C. for 16 h. The completed reaction was quenched at 0° C. with water (150 mL), and then extracted with MTBE (2×500 mL ea). The combined organic layers were washed with 0.3 N HCl (300 mL), then carefully washed with sat. NaHCO₃ (200 mL), 1:1 water: brine (100 mL), dried over Na₂SO₄, filtered, and concentrated. The crude 5-benzoate intermediate was used in next step without purification.

To a stirred solution of crude (3aR,4R,6S,7aR)-6-(((tert-butyldiphenylsilyl)oxy)methyl)-6-hydroxy-2,2-dimethylhexahydrobenzo[d][1,3]dioxol-4-yl benzoate (19.44 g, 34.667 mmol) in water (19.43 mL) at room temperature was added acetic acid (78 mL, 1.36 mol). The reaction mixture was warmed to 70° C., and stirred for 1 h, after which time it was cooled to 0° C., after which time water (400 mL mmol), ethyl acetate (649 mL), and then slowly a portion wise addition sodium bicarbonate (145 g, 1.73 mol). The quenched reaction was stirred at 0° C. for 1 h, after which time the layers were separated, and the aqueous layer was extracted with EtOAc (700 mL). The combined organic layers were washed with 1:1 water: brine (100 mL), dried over Na₂SO₄, filtered, and concentrated. The crude triol intermediate was used in next step without purification.

To a stirred solution of crude (1R,2R,3R,5S)-5-(((tert-butyldiphenylsilyl)oxy)methyl)-2,3,5-trihydroxycyclohexyl benzoate (1.35 g, 2.593 mmol) in acetone (30 mL) and water (15 mL) at room temperature was added sodium periodate (0.832 g, 3.889 mmol). The reaction mixture was stirred for 12 h, after which time sodium periodate (0.277 g, 1.296 mmol) was added, and the reaction mixture was stirred for 16 h. The completed mixture was extracted with MTBE (2×70 mL ea), and the combined organic layers were washed with sat. NaHCO₃ (50 mL), 1:1 water: brine (50 mL), dried over Na₂SO₄, filtered, and concentrated. The crude dialdehyde intermediate was used in next step without purification.

To a stirred solution of crude (2R,4R)-4-(((tert-butyldiphenylsilyl)oxy)methyl)-4-hydroxy-1,6-dioxohexan-2-yl benzoate (1.345 g, 2.593 mmol) in THF (19.3 mL) and methanol (2.72 mL) at 0° C. was added sodium borohydride (0.196 g, 5.186 mmol). The reaction was stirred at 0° C. for 2 h, after which time, it was then diluted with EtOAc (58 mL) and quenched with sat. sodium bicarbonate (40 mL). The layers were separated, and the aqueous layer was extracted with EtOAc (40 mL). The) and the combined organic layers were washed with s 1:1 water: brine (10 mL), dried over Na₂SO₄, filtered, and concentrated. The crude triol intermediate was used in next step without purification.

To a stirred solution of crude (2R,4S)-4-(((tert-butyldiphenylsilyl)oxy)methyl)-1,4,6-trihydroxyhexan-2-yl benzoate (1.28 g, 2.449 mmol) in methanol (27.5 mL) at 0° C. was added a 20% solution of sodium methoxide (0.448 mL, 1.959 mmol). The reaction mixture was stirred at 0° C. for 16 h, after which time Dowex 50W×4 hydrogen form resin (4.4 g) was added, and the suspension was stirred at 0° C. for 5-10 min, then filtered, rinsed the filter pad with MeOH (5 mL), and the filtrate was concentrated to dry. The residue was dissolved in acetonitrile, triethylamine (0.4 mL, 2.87 mmol) was added, and the mixture was concentrated to dry. The final residue was purified over a Biotage Ultra SNAP silica gel column (25 g) eluting with 1 CV 5% EtOAc in heptane, a 2 CV gradient of 5 to 50% EtOAc in heptane, a 10 CV gradient of 50 to 100% EtOAc in heptane, then 2 CV EtOAc to provide compound 107 (0.87 g, 2.078 mmol, 30% overall yield) (MW_{Calc}+Na=441.22; MW_{Obs}=441.30) after collection of the desired fractions, concentration and drying under vacuum.

To a stirred solution of (2R,4S)-4-(((tert-butyldiphenylsilyl)oxy)methyl)hexane-1,2,4,6-tetraol (107, 2.33 g, 5.566 mmol) in DCM (15 mL) at 0° C. was added 2,2-dimethoxypropane (0.80 mL, 6.123 mmol) and p-toluenesulfonic acid monohydrate (0.053 g, 0.278 mmol). The reaction mixture was warmed to room temperature and stirred for 1 h, after which time it was quenched with aq NaHCO₃ (10 mL) followed by the addition of EtOAc (50 mL). The layers were separated, and the aqueous layer was extracted with EtOAc (2×10 mL ea). The combined organic layers were washed with brine (5 mL), dried over Na₂SO₄, filtered, concentrated, and vacuumed to dryness to provide the desired acetonide (2.50 g, 5.45 mmol, 98%) as clear oil, which was used in next step without purification.

To a stirred solution of (S)-4-((tert-butyldiphenylsilyl)oxy)-3-(((R)-2,2-dimethyl-1,3-dioxolan-4-yl)methyl)butane-1,3-diol (2.1 g, 4.578 mmol) in DMSO (11 mL) at room temperature was added IBX (2.56 g, 9.157 mmol). The reaction mixture was stirred for 7 h, after which time the reaction was quenched with an aqueous solution of sodium thiosulfate (2 g) in water (10 mL) and aqueous NaHCO₃ (10 mL). The resulting mixture was diluted with EtOAc (30 mL), and stirred for 5 min, after which time the layers were separated. The aqueous layer was extracted with EtOAc (10 mL), and the combined organic layers were washed with aqueous NaHCO₃ (10 mL), water (10 mL), brine (10 mL), dried over Na₂SO₄, filtered, and concentrated. The final residue was purified over a Biotage Ultra SNAP silica gel column (25 g) eluting with a 10 CV gradient of 0 to 100% EtOAc in heptane to provide pure aldehyde intermediate (1.80 g, 3.94 mmol, 86%) after collection of the desired fractions, concentration and drying under vacuum.

To a stirred solution of (R)-4-((tert-butyldiphenylsilyl)oxy)-3-(((R)-2,2-dimethyl-1,3-dioxolan-4-yl)methyl)-3-hydroxybutanal (1.7 g, 3.723 mmol) in dry 1,2-DCE (17 mL) at room temperature was added ethyl (triphenylphosphoranylidene)acetate (2.59 g, 7.445 mmol). The reaction mixture was warmed at 40° C., stirred for 4 h, after which time it was cooled to room temperature, and then stirred for 16 h. The completed reaction was concentrated to approx. 7 mL, and directly purified over a Biotage Ultra SNAP silica gel column (50 g) eluting with a 10 CV gradient of 0 to 65% EtOAc in heptane to provide compound 108 (1.80 g, 3.42 mmol, 92%) after collection of the desired fractions, concentration and drying under vacuum.

To a stirred solution of ethyl (S,E)-6-((tert-butyldiphenylsilyl)oxy)-5-(((R)-2,2-dimethyl-1,3-dioxolan-4-yl)methyl)-5-hydroxyhex-2-enoate (108, 1.8 g, 3.417 mmol) in ethanol (6 mL) and ethyl acetate (18 mL) at room temperature was added 5% Pd—C(0.364 g, 3.417 mmol) followed by purging with H₂ (3×), and placing under a H₂ atmosphere for 20 h. The reaction mixture was stirred for 20 h, after which time was purged with N2 gas (3×), filtered over a pad of Celite (10 g), rinsed with ethanol (3×10 mL), the filtrate concentrated, and then azeotroped to dryness with toluene (2×10 mL ea). The residue was dissolved with stirring with DCM (10 mL) at room temperature followed by the addition of 2,2-dimethoxypropane (2 mL), and then p-toluenesulfonic acid monohydrate (0.020 g, 0.105 mmol). The reaction mixture was stirred for 20 min, after which time it was quenched with aq NaHCO₃ (10 mL) followed by the addition of EtOAc (20 mL). The layers were separated, and the organic layer was dried over Na₂SO₄, filtered, concentrated, and placed under vacuumed until dry. Obtained the pure saturated intermediate (1.80 g, 3.94 mmol, 100%) (MW_{Calc}+Na=551.29; MW_{Obs}=551.26).

To a stirred solution of ethyl (S)-6-((tert-butyldiphenylsilyl)oxy)-5-(((R)-2,2-dimethyl-1,3-dioxolan-4-yl)methyl)-5-hydroxyhexanoate (1.90 g, 3.593 mmol) in toluene (42.8 mL) at −78° C. was added dropwise 1 M DIBAL-H (8.08 mL, 8.085 mmol) over 10 min. The reaction mixture was stirred at −78° C. for 1.5 h, after which time methanol (500 μL) was slowly added followed by a saturated solution of Rochelle's salts (13 g in water (50 mL)). The quenched reaction was stirred at room temperature for 3 h, after which time it was extracted with EtOAc (3×50 mL ea). The combined organic layers were washed with water (50 mL), brine (50 mL), dried over Na₂SO₄, filtered, concentrated, and placed under vacuumed until dry. The resultant aldehyde/lactal (1.68 g, 3.47 mmol, 96%) (MW_{Calc}+Na=507.26; MW_{Obs}=507.23) was used in next step without further purification.

To a stirred solution of ethyl (S,E)-8-((tert-butyldiphenylsilyl)oxy)-7-(((R)-2,2-dimethyl-1,3-dioxolan-4-yl)methyl)-7-hydroxyoct-2-enoate (1.8 g, 3.244 mmol) in THF (25 mL) 0° C. was added 1.0 M potassium tert-butoxide in THF (0.324 ml, 0.324 mmol). The reaction mixture was stirred for 10 min, after which time it was quenched with a mixture of aq NH₄Cl (25 mL) and EtOAc (25 mL). The layers were separated, and the aqueous layer was extracted with EtOAc (2×20 mL ea). The combined organic layers were washed with brine (5 mL), dried over Na₂SO₄, filtered, concentrated, and vacuumed to dryness to provide the desired pyran intermediate (1.31 g, 2.36 mmol, 73%) (MW_{Calc}+Na=57.31; MW_{Obs}=577.27) as clear oil in a 1:1 ratio of epimers, which was sufficiently pure to use in next step without purification.

To a stirred solution of ethyl 2-((6S)-6-(((tert-butyldiphenylsilyl)oxy)methyl)-6-(((R,S)-2,2-dimethyl-1,3-dioxolan-4-yl)methyl)tetrahydro-2H-pyran-2-yl)acetate (1.81 g, 3.262 mmol) in toluene (54.3 mL) at −78° C. was added dropwise 1 M DIBAL-H in THF (3.43 mL, 3.426 mmol) over 20 min, after which time it was stirred at −78° C. for 1 h. The completed reaction was carefully quenched with a dropwise addition of MeOH (0.5 mL) followed by a saturated solution of Rochelle's salts (10 g in water (40 mL)). The resulting mixture was stirred at room temperature for 1 h, after which time the layers were separated, and the aqueous layer was extracted with EtOAc (3×50 mL ea). The combined organic layers were washed with water (50 mL), brine (50 mL), dried over Na₂SO₄, filtered, and concentrated. The residue was purified over a Biotage Ultra SNAP silica gel column (100 g) eluting with 2 CV heptane, a 12 CV gradient of 0 to 12.5% MTBE in heptane, and then 3 CV 12.5% MTBE in heptane to provide compound 109 (600 mg, 1.175 mmol, 36%) (MW_{Calc}+Na=533.28; MW_{Obs}=533.23), and 110 (502 mg, 0.983 mmol, 30%) (MW_{Calc}+Na=533.28; MW_{Obs}=533.23) compound after collection of the desired fractions, concentration and drying under vacuum.

To a stirred solution of (methyl)triphenylphosphonium bromide (1.749 g, 4.895 mmol) in THF (20 mL) at 0° C. was added dropwise 1.5 M n-BuLi in THF (2.87 mL, 4.307 mmol) over 5 min. The mixture was stirred for 10 min, after which time it was cooled to −78° C. followed by a dropwise addition of a solution of 2-((2R,6S)-6-(((tert-butyldiphenylsilyl)oxy)methyl)-6-(((R)-2,2-dimethyl-1,3-dioxolan-4-yl)methyl)tetrahydro-2H-pyran-2-yl)acetaldehyde (110, 1.0 g, 1.958 mmol) in THF (10 mL) over 10 min. The reaction mixture was stirred for 5 min, after which time it was warmed to room temperature, and stirred for 4 h. The reaction mix was diluted with a 1:2 ratio of MTBE:heptane (50 mL) followed by the addition of silica gel (5 g). The slurry was filtered over a pad of silica gel (50 g) rinsing several times with MTBE until all desired compound was eluted. The filtrate was concentrated dryness to provide the desired terminal olefin as an oil that was sufficiently pure to be used in the next step.

To a stirred solution of (((2S,6R)-6-allyl-2-(((R)-2,2-dimethyl-1,3-dioxolan-4-yl)methyl)tetrahydro-2H-pyran-2-yl)methoxy)(tert-butyl)diphenylsilane (300 mg, 0.59 mmol) in THF (1 mL) at room temperature was added 1.0 M TBAF in THF (1.179 mL, 1.179 mmol). The reaction was stirred for 28 h, after which time the reaction mixture was diluted with EtOAc (20 mL), and the organic layer was washed with water (3×5 mL), with brine (5 mL), dried over Na2SO4, filtered, and concentrated to dry. The resultant primary alcohol was dissolved with stirring in DCM (6 mL) at room temperature followed by the addition of pyridine (52.5 μL, 0.649 mmol), and then Dess-Martin periodinane (388 mg, 0.914 mmol). The intermediate reaction was stirred for 2 h, after which time it was diluted with EtOAc (20 mL), washed with sat. aqueous NaHCO₃ (5 mL), sodium thiosulfate (5 mL), followed by NaHCO₃ (5 mL) and brine (5 mL). The organic layer was concentrated to dry, after which time it was diluted with t-BuOH (2 mL), pH7 buffer (2 mL) and 2-methyl-2-butene (2 mL) at room temperature. To the resulting mixture was added sodium chlorite (107 mg, 1.179 mmol) followed by stirring at room temperature for 16 h. The completed reaction was quenched with 1 N HCl (0.2 mL) followed by sat NH₄Cl (3 mL). The mixture was extracted with EtOAc (3×5 mL ea), and the combined organic layers were washed with brine (5 mL), and then concentrated. The resultant acid intermediate was dissolved with stirring in MeOH (0.6 mL) and toluene (4 mL) at room temperature followed by the addition of 2 M trimethylsilyl-diazomethane in ethyl ether (590 μL, 1.179 mmol). The final reaction mixture was stirred for 5 min, and then concentrated after remaining reagent is consumed. The final residue was purified over a Biotage Ultra SNAP silica gel column (10 g) eluting with a 10 CV gradient of 0 to 35% EtOAc in heptane to provide compound 111 (147 mg, 0.493 mmol, 84%) after collection of the desired fractions, concentration and drying under vacuum.

A previously stirred homogeneous suspension of potassium carbonate (167 mg, 1.207 mmol), potassium hexacyanoferrate(III) (397 mg, 1.207 mmol), (DHQ)₂PYR (23.76 mg, 0.027 mmol) and potassium osmate(VI) dihydrate containing 51.0-52.0% Os (2.96 mg, 8.043 μmol) in a mixture of t-BuOH (3 mL) and water (3 mL) generated at room temperature and stirred for 10 min was added to a cooled mixture of methyl (2S,6R)-6-allyl-2-(((R)-2,2-dimethyl-1,3-dioxolan-4-yl)methyl)tetrahydro-2H-pyran-2-carboxylate (111, 120 mg, 0.402 mmol) in a solution of tBuOH (3 mL) and water (3 mL) at 5° C. The reaction mixture was stirred at 0° C. for 4 h, after which time it was diluted with EtOAc (20 mL) followed by the addition of sat. aq sodium thiosulfate (10 mL). The quenched reaction was stirred for 10 min, after which time the layers were separated and the organic layer was washed with sat. aq NH₄Cl (2×5 mL), and brine (5 mL). The organic layer dried over Na₂SO₄, filtered, and concentrated to provide the crude diol in approx. 3:1 ratio of isomers.

To a stirred solution of the crude diol in pyridine (1 mL) at room temperature was added acetic anhydride (1 mL). The reaction mixture was stirred for 4 h, after which time the completed reaction was diluted with EtOAc (10 mL) and washed with sat. aq NH₄Cl (5 mL), sat. aq NaHCO₃ (5 mL), and brine (5 mL). The organic layer dried over Na₂SO₄, filtered, and concentrated. The resulting residue was purified over a Biotage Ultra SNAP silica gel column (10 g) eluting with a 10 CV gradient of 0 to 100% EtOAc in heptane to provide compound 112 (145 mg, 0.348 mmol, 87%) (MW_{Calc}+Na=439.20; MW_{Obs}=439.22) after collection of the desired fractions, concentration and drying under vacuum.

To a stirred solution of (S)-3-((2R,6S)-6-(((R)-2,2-dimethyl-1,3-dioxolan-4-yl)methyl)-6-(methoxycarbonyl)tetrahydro-2H-pyran-2-yl)propane-1,2-diyl diacetate (112, 130 mg, 0.312 mmol) in ethyl acetate (2.5 mL) at room temperature was added periodic acid (213 mg, 0.936 mmol). The reaction mixture was stirred at for 7 h, after which time it was quenched with 10% aq Na₂S₂O₃ (2 mL) and 10% aq NaHCO₃ (2 mL) followed by stirring for 5 min. The mixture was extracted with EtOAc (2×5 mL ea), and the combined organic layers were washed with sat. aq NaHCO₃ (2 mL), and brine (2 mL). The organic layer was dried over Na₂SO₄, filtered, concentrated and the resulting residue was filtered over a pad of silica gel (10 g) eluting with EtOAc (100 mL total). The eluent was concentrated to dry to provide the compound 113 (102 mg, 0.296 mmol, 95%), which was used in the next step without further purification.

To a stirred solution of (S)-3-((2R,6S)-6-(methoxycarbonyl)-6-(2-oxoethyl)tetrahydro-2H-pyran-2-yl)propane-1,2-diyl diacetate (113, 65 mg, 0.189 mmol) in 1,2-DCE (1.3 mL) was added tert-butyl (S)-9-oxa-2,6-diazaspiro[4.5]decane-2-carboxylate (19, 68.6 mg, 0.283 mmol), acetic acid (54.0 μl, 0.944 mmol) and dried 4A molecular sieves (200 mg). The suspension was stirred for 2 h, after which time sodium triacetoxyborohydride (80 mg, 0.378 mmol) was added and stirred for an additional 1 h. The reaction mixture was quenched with aq NaHCO₃ (5 mL) followed by the addition of EtOAc (5 mL). The layers were separated, and the organic layer was washed with brine (5 mL), dried over Na₂SO₄, filtered, concentrated, and purified over a Biotage Ultra SNAP silica gel column (4 g) eluting with a 10 CV gradient of 0 to 100% EtOAc in heptane to provide the diacetyl intermediate (78 mg, 0.137 mmol, 72%) after collection of the desired fractions, concentration and drying under vacuum. The product was dissolved in MeOH (3 mL), cooled to 0° C., followed by the addition of sodium methoxide (40.8 mg, 0.189 mmol). The reaction mixture was stirred for 3 h, after which time silica gel (5 g) and MTBE (5 mL) were added. The slurry was filtered over a plug of Celite (5 g) and eluted with EtOAc (20 mL total). The filtrated was concentrated to provide the resulting diol (64 mg, 0.132 mmol, 96%) (MW_{Calc}+Na=509.29; MW_{Obs}=509.31) and used as is in next step without further purification.

To a stirred solution of tert-butyl (S)-6-(2-((2S,6R)-6-((S)-2,3-dihydroxypropyl)-2-(methoxycarbonyl)-tetrahydro-2H-pyran-2-yl)ethyl)-9-oxa-2,6-diazaspiro[4.5]decane-2-carboxylate (64 mg, 0.132 mmol) in DCM (1.6 mL) at 0° C. was added pyridine (0.053 mL, 0.658 mmol), and a dropwise solution of p-toluenesulfonic anhydride (42.9 mg, 0.132 mmol) in DCM (0.5 mL) over 3 min. The mixture was stirred for 3 h, after which time it was diluted with EtOAc (5 mL), and aq NaHCO₃ (5 mL) followed by stirring for an additional 1 h. The layers were separated, and the organic layer was washed with NaHCO₃ (3 mL), brine (3 mL), and concentrated to dryness. The resulting residue was azeotroped to dry with acetone (2×5 mL ea), followed by dissolving with stirring in acetone (1 mL) and water (0.2 mL) followed by the addition of sodium azide (86 mg, 1.315 mmol). The reaction mixture was warmed to 65° C. and stirred for 22 h. The completed reaction was cooled to room temperature, diluted with water (5 mL), and extracted with EtOAc (4×5 mL ea). The combined organic layers were washed with sat. aq NaHCO₃ (5 mL), brine (5 mL), dried over Na₂SO₄, filtered and concentrated. The resulting residue was purified over a Biotage Luknova silica gel column (4 g) eluting with a 10 CV gradient of 0 to 100% EtOAc in heptane to provide compound 114 (54 mg, 0.106 mmol, 80%) (MW_{Calc}+Na=534.30; MW_{Obs}=534.31) after collection of the desired fractions, concentration and drying under vacuum.

To a stirred solution of tert-butyl (S)-6-(2-((2S,6R)-6-((S)-3-azido-2-hydroxypropyl)-2-(methoxycarbonyl)tetrahydro-2H-pyran-2-yl)ethyl)-9-oxa-2,6-diazaspiro[4.5]decane-2-carboxylate (114, 100 mg, 0.195 mmol) in THF (2 mL) and water (0.2 mL) at room temperature was added trimethylphosphine (538 μl, 0.538 mmol). The reaction mixture was stirred for 8 h, after which time the reaction mixture was evaporated, and azeotroped to dry (3×10 mL ea). The residue was divided in two equal portions A and B. Portion A was dissolved with stirring DMAC (1 mL) at room temperature followed by the addition of 7-methyl-1H-indole-5-carboxylic acid (22.26 mg, 0.127 mmol) triethylamine (34.1 μL, 0.244 mmol), HOBt (5.99 mg, 0.039 mmol) and EDC (28.1 mg, 0.147 mmol). The reaction mixture was stirred at room temperature for 16 h. The completed reaction was diluted with water (5 mL) and extracted with EtOAc. (3×5 mL ea). The combined organic layers were washed with sat. aq NaHCO₃ (5 mL), water, (5 mL), brine (5 mL), dried over Na₂SO₄, filtered and concentrated. One third of the residue was purified by HPLC method to provide A-206 (9.34 mg, 0.15 mmol, 22%) (MW_{Calc}+H=643.37; MW_{Obs}=644.62) after collection of the desired fractions, concentration and drying under vacuum. The remaining two thirds of the residue of A-206 was used in the next reaction without further purification.

Compound 115 was prepared in a similar to A-206 starting with portion B from above using 4-hydroxy-3,5-dimethylbenzoic acid (21.11 mg, 0.127 mmol) instead of 7-methyl-1H-indole-5-carboxylic acid to provide 115. One third of the residue was purified by HPLC method to provide 115 (9.31 mg, 0.014 mmol, 22%) (MW_{Calc}+H=656.36; MW_{Obs}=656.28) after collection, concentration and drying under vacuum. The remaining two thirds of the residue of 115 was used in the next reaction without further purification.

To a stirred solution of tert-butyl (S)-6-(2-((2S,6R)-6-((S)-2-hydroxy-3-(7-methyl-1H-indole-5-carboxamido)propyl)-2-(methoxycarbonyl)tetrahydro-2H-pyran-2-yl)ethyl)-9-oxa-2,6-diazaspiro[4.5]decane-2-carboxylate (A-206, 35 mg, 0.054 mmol) in MeOH (0.6 mL) at room temperature was added 1 N aqueous sodium hydroxide (544 μL, 0.544 mmol). The reaction mixture was stirred for 72 h, after which time it was neutralized with 1 N HCl (0.5 mL) and submitted directly for HPLC purification to provide A-207 (16 mg, 0.025 mmol, 47%) (MW_{Calc}+H=629.35; MW_{Obs}=630.30) after collection of the desired fractions, concentration and drying under vacuum.

A-208 was prepared in a similar fashion to A-207 starting with 115 (34.5 mg, 0.054 mmol) to provide A-208 (16 mg, 0.026 mmol, 48%) (MW_{Calc}+H=620.35; MW_{Obs}=620.86).

### Preparation of A-209 to A-212

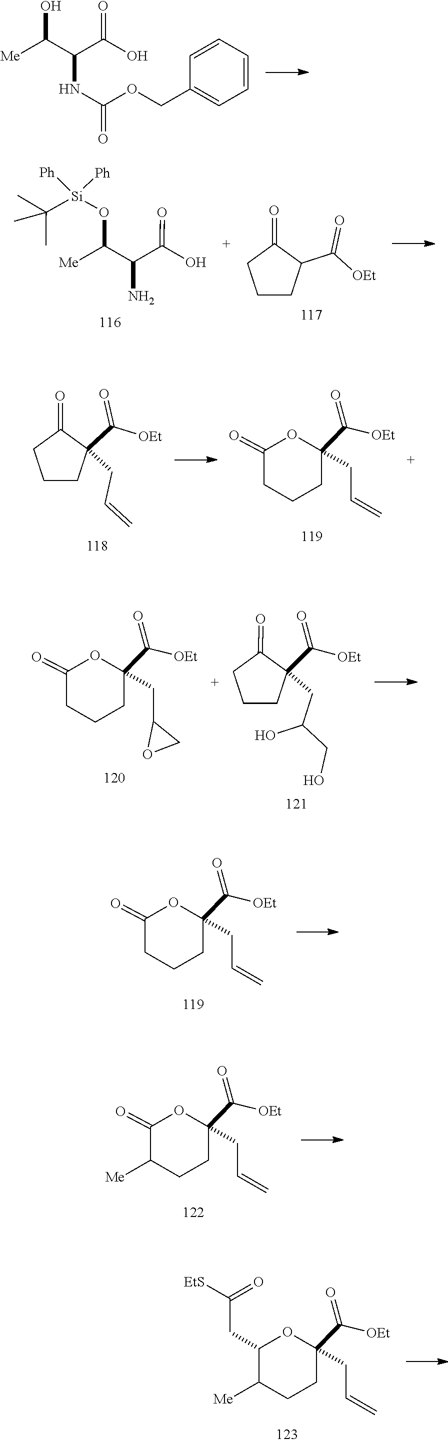

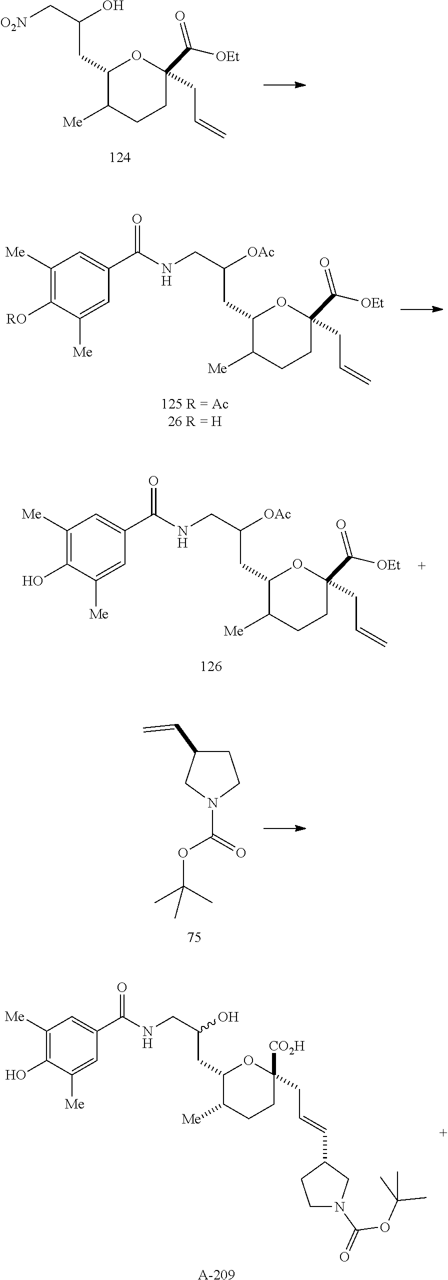

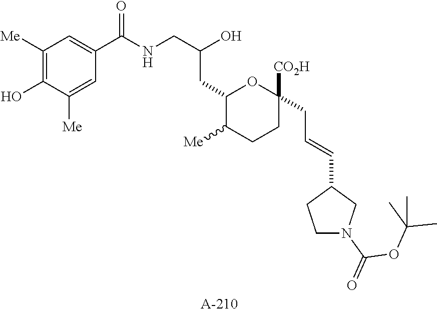

To a stirred solution of ((benzyloxy)carbonyl)-L-threonine (20 g, 78.97 mmol) in DCM (200 mL) at room temperature were added imidazole (26.9 g, 394.86 mmol) and TBDPS-C1 (40.6 ml, 157.944 mmol). The mixture was stirred for 16 h, after which time the completed intermediate reaction was diluted with water (30 mL) and extracted with EtOAc (3×50 mL ea). The combined organic layers were dried over Na₂SO₄ and filtered over a pad of silica gel (20 g) eluting with MTBE (250 mL). The combined filtrates were concentrated to dry and dissolved with stirring in MeOH (400 mL) at room temperature, purged with a N2 atmosphere (3×) followed by the addition of 10% Pd—C(8.40 g, 3.949 mmol). The reaction mixture was purged with H2 (5×), placed under balloon pressure, and stirred at room temperature for 16 h. The completed reaction was purged with N2 (5×), diluted with MTBE (100 mL), filtered over a pad of Celite (20 g), eluting with MTBE (200 mL). The filtrates were concentrated and dried under vacuum to provide compound 116 (15 g, 42.0 mmol, 53%) (MW_{Calc}+Na=380.18; MW_{Obs}=380.20) as a white solid.

To a stirred solution of palladium(II) acetate (2.52 g, 11.205 mmol) in toluene (420 mL) at room temperature was added tris(4-fluorophenyl)phosphine (10.63 g, 33.615 mmol) and O-(tert-butyldiphenylsilyl)-L-allothreonine (116, 16.02 g, 44.82 mmol) followed by allyl acetate (48.4 ml, 448.2 mmol) and ethyl 2-oxocyclopentanecarboxylate (117, 33.2 mL, 224.099 mmol). The reaction was stirred at room temperature for 72 h, after which time it was concentrated to 50% the original volume, then purified directly over Biotage Ultra SNAP silica gel column (340 g) eluting with a 10 CV gradient of 0 to 60% EtOAc in heptane to provide compound 118 (42 g, 214 mmol, 96% and 96% ee via chiral HPLC) after collection of the desired fractions, concentration and drying under vacuum.

To ethyl (R)-1-allyl-2-oxocyclopentane-1-carboxylate (118, 28 g, 142.68 mmol), oven dried 4A molecular sieves (5 g), and sodium bicarbonate (23.97 g, 285.36 mmol) was added dropwise at room temperature with stirring m-CPBA (0.251 g, 1.121 mmol) in DCM (280 mL) over a 1 h period. The reaction mixture was stirred for 24 h, after which time it was filtered over Celite (30 g), eluted with EtOAc (100 mL). The combined filtrate was washed with basic water (pH 10, 2×20 mL ea), and (20 mL×2) and saturated NaHCO₃ (20 mL). The organic layers were concentrated to dry followed by dilution with chloroform (150 mL), addition of hexafluoro-2-propanol (150 mL), and water (30 mL). The final mixture warmed to 60° C. and stirred for 24 h. The completed mixture was concentrated to dryness and then purified directly over a Biotage Ultra SNAP silica gel column (340 g) eluting with a 10 CV gradient of 5 to 80% EtOAc in heptane to provide compounds 119 (13 g, 61.2 mmol, 43%) (MW_{Calc}+Na=235.10; MW_{Obs}=235.22), 120 (5 g, 21.9 mmol, 15%) (MW_{Calc}+Na=251.10; MW_{Obs}=251.18), and 121 (8.5 g, 36.9 mmol, 26%) (MW_{Calc}+Na=253.12; MW_{Obs}=253.20) after collection of the desired fractions, concentration and drying under vacuum.

To a stirred solution of diisopropylamine (2.216 mL, 15.548 mmol) in THF (30.0 mL) at 0° C. was dropwise 1.5 M n-BuLi in THF (9.72 ml, 15.548 mmol) over a 10 min period. The mixture was stirred at 0° C. for 1 h, after which time the mixture was cooled to −78° C. followed by a dropwise addition of ethyl (S)-2-allyl-6-oxotetrahydro-2H-pyran-2-carboxylate (119, 3 g, 14.135 mmol) in THF (5 mL) over 5 min. The reaction mixture was stirred at −78° C. for 30 min, after which time HMPA (2.95 ml, 16.962 mmol) was slowly added followed by a dropwise addition of a solution of methyl iodide (0.972 mL, 15.548 mmol) in THF (5 mL) at −78° C. over 5 min. The final reaction mixture was allowed to warm to −20° C. over 5 h. The completed reaction was quenched with sat. NaHCO₃ (5 mL), warmed to room temperature, and then extracted with MTBE (3×5 mL ea). The combined organic layers were dried over Na2SO4, filtered, concentrated and purified over a Biotage Ultra SNAP silica gel column (25 g) eluting with a 10 CV gradient of 5 to 50% EtOAc in heptane to provide compounds 122 (1.7 g, 7.51 mmol, 53%) (MW_{Calc}+Na=249.12; MW_{Obs}=249.21) after collection of the desired fractions, concentration and drying under vacuum.

To a stirred solution of ethyl (2S)-2-allyl-5-methyl-6-oxotetrahydro-2H-pyran-2-carboxylate (122, 1.6 g, 7.07 mmol) in THF (20.80 mL) at −78° C. was added dropwise 1 M DIBAL-H in toluene (17.68 mL, 17.68 mmol) over a 15 min period. The reaction was stirred at −78° C. for 1 h, warmed to −20° C., and stirred for 16 h. The completed intermediate reaction was quenched slowly with MeOH (0.5 mL) and then saturated sodium potassium tartrate (5 mL), warmed to room temperature, and stirred for 30 min. The resulting mixture was extracted with EtOAc (3×4 mL ea), and the combined organic layers were dried over Na₂SO₄, filtered, and concentrated to provide the intermediate aldehyde.

To a stirred solution of dried lithium chloride (0.659 g, 15.556 mmol) in THF (20.80 mL) at 0° C. was added S-ethyl 2-(diethoxyphosphoryl)ethanethioate (2.209 g, 9.192 mmol) and Et₃N (1.971 ml, 14.142 mmol). The mixture was stirred for 15 min, after which time was added a solution of the crude aldehyde in THF (10 mL) over 5 min. The final reaction mixture was stirred at 0° C. for 3 h, after which time the mixture was diluted with DCM (100 mL) and DBU (1.066 mL, 7.071 mmol). The reaction was slowly warmed to room temperature and stirred for 24 h. The completed reaction was quenched with sat.

NaHCO₃ (3 mL) and extracted with EtOAc (3×4 mL ea). The combined organic layers were dried over Na₂SO₄, filtered, concentrated and purified over a Biotage Ultra SNAP silica gel column (10 g) eluting with a 10 CV gradient of 0 to 50% EtOAc in heptane to provide compound 123 (1.1 g, 3.50 mmol, 50%) (MW_{Calc}+Na=337.16; MW_{Obs}=337.50) after collection of the desired fractions, concentration and drying under vacuum.

To a stirred solution of ethyl (2S)-2-allyl-6-(2-(ethylthio)-2-oxoethyl)-5-methyltetrahydro-2H-pyran-2-carboxylate (123, 1.05 g, 3.339 mmol) in THF (43.2 mL) at −78° C. was added dropwise 1 M DIBAL-H in toluene (11.13 mL, 11.13 mmol) over 10 min. The reaction mixture was stirred at −78° C. for 2 h, after which time it was slowly quenched with MeOH (0.5 mL) and then saturated sodium potassium tartrate (5 mL), warmed to room temperature, and stirred for 30 min. The resulting mixture was extracted with MTBE (2×30 mL ea), and the combined organic layers were dried over Na₂SO₄, filtered, and concentrated to provide the crude intermediate aldehyde. The crude aldehyde was dissolved with stirring in DCM (18.9 mL) at room temperature followed by the addition of nitromethane (3.6 mL, 66.78 mmol) and triethylamine (1.40 mL, 10.02 mmol). The final reaction mixture was stirred for 16 h, after which time it was quenched with sat. NaHCO₃ (5 mL) and extracted with EtOAc (3×40 mL ea). The combined organic layers were dried over Na2SO4, filtered, concentrated and purified over a Biotage Ultra SNAP silica gel column (25 g) eluting with a 10 CV gradient of 5 to 70% MTBE in EtOAc to provide compounds 124 (0.9 g, 2.85 mmol, 85%) (MW_{Calc}+Na=338.17; MW_{Obs}=338.17) after collection of the desired fractions, concentration and drying under vacuum.

To a stirred solution of ethyl (2S)-2-allyl-6-(2-hydroxy-3-nitropropyl)-5-methyltetrahydro-2H-pyran-2-carboxylate (124, 0.15 g, 0.476 mmol) in EtOH (3.00 mL) at room temperature were added saturated NH₄Cl (0.4 mL) and zinc (0.187 g, 2.854 mmol). The reaction mixture was warmed to 80° C. and stirred for 3 h. The completed intermediate reaction was cooled to room temperature, diluted with EtOAc (6 mL), filtered over a pad of Celite (10 g), and eluted with EtOAc (20 mL). The combined filtrates were concentrated to dry, after which time the resulting residue was diluted with stirring with MeCN (2.250 mL) and DMA (0.450 mL) at room temperature followed by the addition of 4-hydroxy-3,5-dimethylbenzoic acid (0.119 g, 0.713 mmol), HOBt (0.036 g, 0.238 mmol), and EDC (0.128 g, 0.666 mmol). Et₃N (0.199 ml, 1.427 mmol) was added to the reaction mixture followed by stirring at room temperature for 16 h. The completed reaction was diluted with DCM (3 mL) at room temperature followed by the addition of Et3N (1 mL), Ac₂O (0.5 mL) and DMAP (20 mg). The final reaction mixture was stirred at room temperature for 3 h, after which time it was quenched with water (5 mL) and extracted with EtOAc (3×5 mL ea). The combined organic layers were dried over Na2SO4, filtered, concentrated and purified over a Biotage Ultra SNAP silica gel column (10 g) eluting with a 10 CV gradient of 5 to 100% EtOAc in heptane to provide compounds 125 (160 mg, 0.309 mmol, 65%) (MW_{Calc}+Na=498.26; MW_{Obs}=498.67), and 126 (50 mg, 0.115 mmol, 24%) (MW_{Calc}+Na=540.27; MW_{Obs}=540.30) after collection of the desired fractions, concentration and drying under vacuum.

To a stirred solution of ethyl (2S,6S)-6-(2-acetoxy-3-(4-hydroxy-3,5-dimethylbenzamido)propyl)-2-allyl-5-methyltetrahydro-2H-pyran-2-carboxylate (126, 50 mg, 0.105 mmol) and tert-butyl (R)-3-vinylpyrrolidine-1-carboxylate (75, 41.5 mg, 0.21 mmol) in DCE (828 μL, 10.513 mmol) at room temperature was added Hoveyda-Grubbs catalyst 2nd generation (13.22 mg, 0.021 mmol) and p-benzoquinone (4.55 mg, 0.042 mmol). The reaction mixture was warmed to 90° C. and stirred for 3 h followed by the addition of DMSO (0.1 mL) then stirred for 16 h at 90° C. The completed reaction was cooled to room temperature and concentrated to approximately one half of the original volume. The mixture was directly purified over a Biotage Ultra SNAP silica gel column (50 g) eluting with a 10 CV gradient of 30 to 100% EtOAc in heptane. The fractions containing the desired intermediate product was combined and concentrated to dry. The resultant residue was dissolved with stirring in MeOH (1.06 mL) and THF (1.03 mL) at room temperature followed by the addition of 1 N aq. NaOH (1.577 mL, 1.577 mmol). The final reaction was stirred for 24 h, after which time the completed reaction was purified directly over an HPLC column to provide A-209 (4.4 mg, 0.0077 mmol, 7%) (MW_{Calc}+H=575.33; MW_{Obs}=575.48) as a mixture of C-8 epimers, and A-210 (10.7 mg, 0.018 mmol, 18%) (MW_{Calc}+H=575.33; MW_{Obs}=575.48) as a mixture of C-5 epimers.

A-211 and A-212 were prepared in a similar manner to A-209 and A-210 starting with compound 124 (0.15 g, 0.476 mmol) to provide A-211 (3.6 mg, 0.006 mmol, 10%) (MW_{Calc}+H=584.33; MW_{Obs}=584.30) as a mixture of 2 isomers (C-8 epimers), and A-212 (0.7 mg, 0.001 mmol, 2%) (MW_{Calc}+H=584.33; MW_{Obs}=584.30) as a mixture of 3 isomers (C-5 and C-8 epimers) after performing similar steps at the appropriate scale and final HPLC purification.

### Preparation of A-213, A-214, and A-215

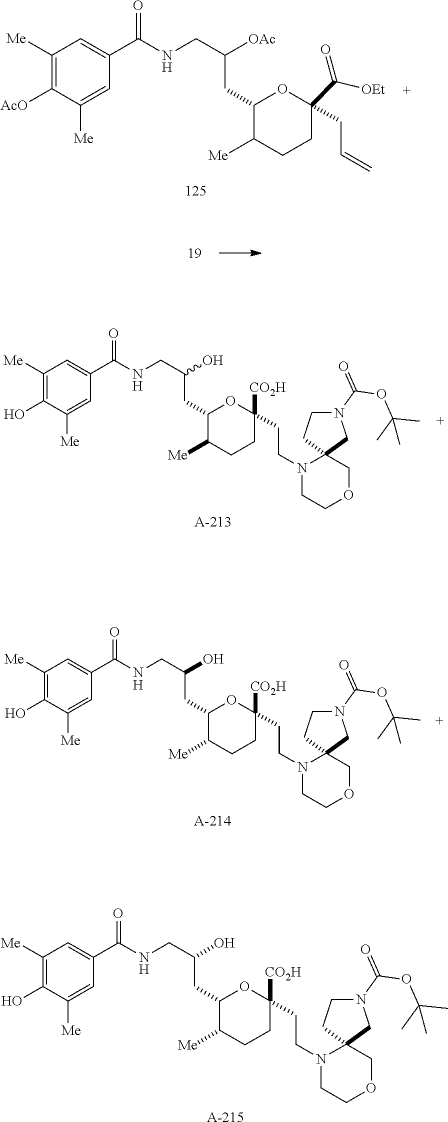

To a stirred solution of ethyl (2S,6S)-6-(2-acetoxy-3-(4-acetoxy-3,5-dimethylbenzamido)propyl)-2-allyl-5-methyltetrahydro-2H-pyran-2-carboxylate (125, 0.10 g, 0.193 mmol) in 1,4-dioxane (3.00 mL) and water (0.600 mL) at room temperature was added 2,6-lutidine (0.045 ml, 0.386 mmol), osmium tetroxide (0.025 ml, 3.864 μmol), and sodium periodate (0.165 g, 0.773 mmol). The reaction mixture was stirred for 3 h, after which time it was diluted with EtOAc (3 mL) and water (2 mL). The layers were separated, and the aqueous was extracted with EtOAc (3×4 mL ea). The combined organic layers were dried over Na2SO4, filtered, and concentrated. The crude resultant aldehyde was diluted with stirring in DCE (1.500 mL) followed by acetic acid (0.077 mL, 1.352 mmol), tert-butyl (S)-9-oxa-2,6-diazaspiro[4.5]decane-2-carboxylate (19, 0.047 g, 0.193 mmol), 4A molecular sieves (386 mg). The suspension was stirred for 2 h, after which time sodium triacetoxyborohydride (0.082 g, 0.386 mmol) was added. The reaction mixture was stirred for 24 h, after which time it was quenched with sat. NaHCO₃ (2 mL) and extracted with EtOAc (3×3 mL ea). The combined organic layers were dried over Na2SO4, filtered, concentrated and purified over Biotage Ultra SNAP silica gel column (10 g) eluting with a 10 CV gradient of 30 to 100% EtOAc in heptane, and then to provide a 5 CV gradient of 0 to 30% MeOH. The desired fractions were collected, and concentrated. The resulting residue was dissolved with stirring in MeOH (2.000 mL) and THF (2.000 mL) followed by the addition of 1 N aqueous NaOH (3.86 mL, 3.864 mmol). The final reaction was stirred for 24 h, warmed to 50° C., and stirred for 16 h. The completed reaction was cooled to room temperature and submitted for HPLC purification to provide A-213 (27 mg, 0.042 mmol, 22%) (MW_{Calc}+H=634.37; MW_{Obs}=634.55), A-214 (9.7 mg, 0.015 mmol, 8%) (MW_{Calc}+H=634.37; MW_{Obs}=634.55), and A-215 (9.7 mg, 0.015 mmol, 8%) (MW_{Calc}+H=634.37; MW_{Obs}=634.55) after collection of the individual desired fractions, concentration and vacuum to dryness.

### Preparation of A-216 to A-219

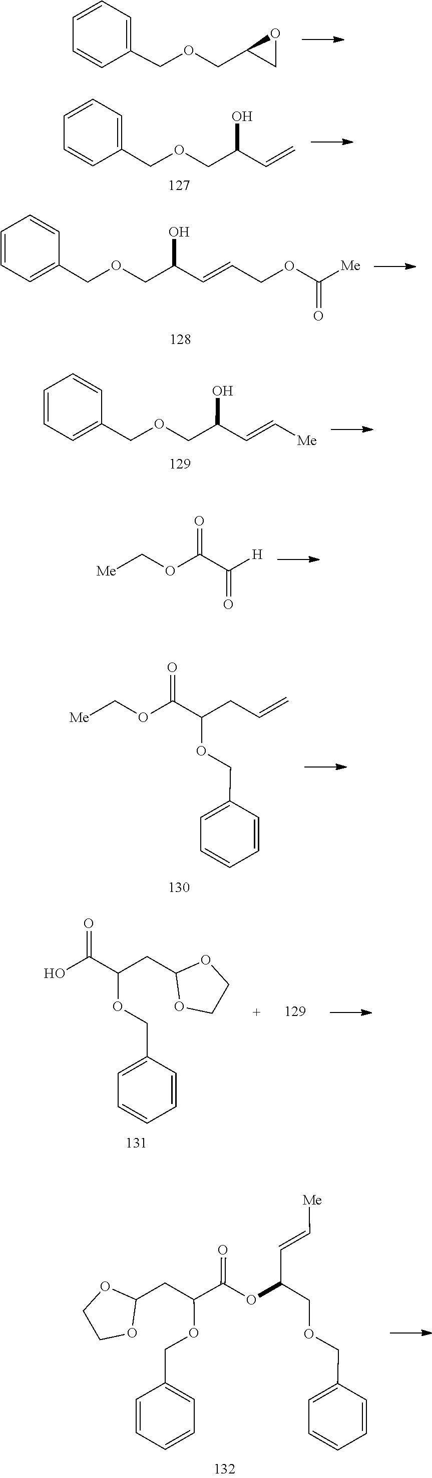

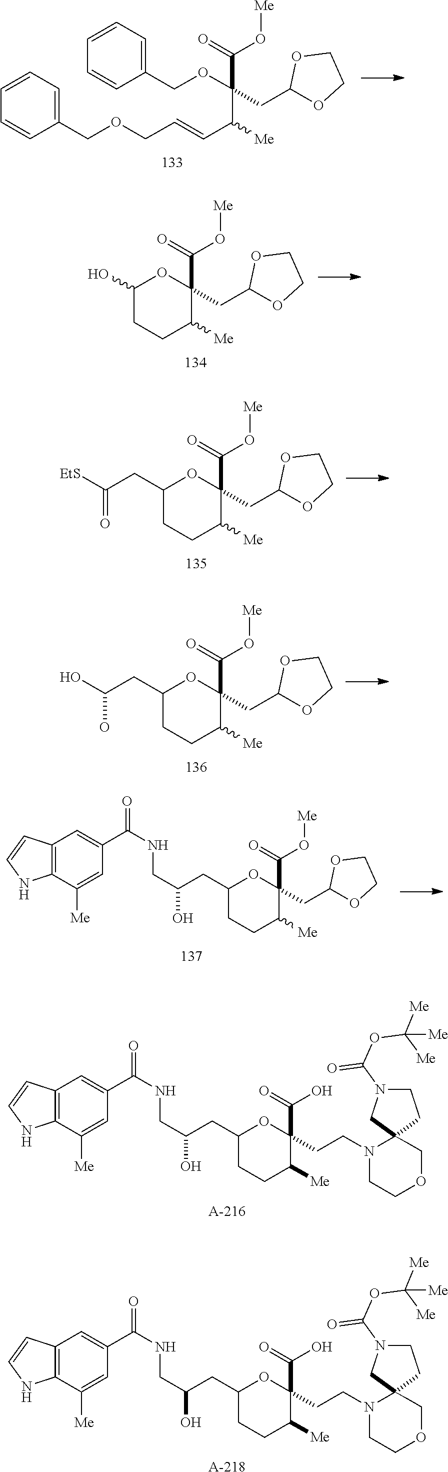

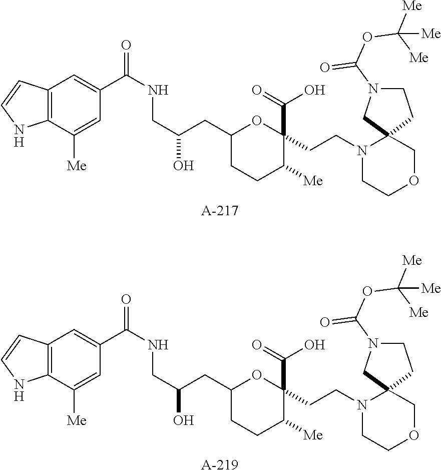

To a stirred solution of trimethylsulfonium iodide (33.0 g, 161.5 mmol) in THF (180 mL) at −30° C., was added dropwise 1.6 M n-BuLi in THF (101 mL, 161.5 mmol) over 30 min, after which time the mixture was stirred at −30° C. for 30 min. (S)-2-((benzyloxy)methyl)oxirane (12.0 g, 73.1 mmol) was added piecemeal, followed by slowly warming to room temperature over 3 h, and then stirring for an additional 16 h. The completed reaction mixture was quenched with addition of water (100 mL) and diluted with heptane (100 mL). The layers were separated, and the aqueous layer was extracted with a 1:1 mixture of heptane:EtOAc (3×50 mL ea). The combined organic layers were dried over Na₂SO₄, filtered, and concentrated. The resulting yellow oil was purified over a 50 g silica gel eluting with heptane (1 L), 5% EtOAc in heptane (1 L), and 5% EtOAc in heptane (1.5 L) collecting 250 mL fractions. The desired fractions were combined, concentrated, and dried under vacuum to provide compound 127 (10.44 g 58.6 mmol, 80%).

A stirred solution of (S)-1-(benzyloxy)but-3-en-2-ol (127, 1.680 g, 9.426 mmol) and allyl acetate (5.08 ml, 47.13 mmol) in DCM (50.4 mL) at room temperature was purged with nitrogen for 30 min, after which time Grubbs Catalyst 2nd Generation (0.080 g, 0.094 mmol) was added. The reaction mixture was warmed to reflux and stirred for 6 h. The completed reaction was cooled to room temperature followed by the addition of tris(hydroxymethyl)phosphine (0.234 g, 1.885 mmol), and stirred for an additional 4 h. The final mixture was diluted with Heptane (100 mL) followed by water (50 mL). The aqueous layer was extracted with a 1:1 mixture of heptane:EtOAc (3×25 mL ea). The combined organic layers were washed with water (25 mL), then brine (25 mL), dried over Na₂SO₄, filtered, and concentrated. The residue was purified over an over Biotage Ultra SNAP silica gel column (25 g) eluting with a 10 CV gradient of 10 to 50% EtOAc in heptane to provide compounds 128 (1.30 g, 0.5.19 mmol, 55%) after collection of the desired fractions, concentration and drying under vacuum.

To a stirred solution of (S,E)-5-(benzyloxy)-4-hydroxypent-2-en-1-yl acetate (1.70 g, 6.79 mmol) in THF (20 mL) at 0° C. was added imidazole (128, 1.156 g, 16.98 mmol) followed by TBS-CI (1.280 g, 8.49 mmol). The reaction mixture was stirred for 1 h, after which time it was quenched with 1:1 ratio of ice:sat. NaHCO₃ and stirred for 30 min warming to room temperature. The mixture was extracted with MTBE (3×10 mL ea), and the combined organic layers were washed with water (10 mL), brine (10 mL), dried over dried over Na₂SO₄, filtered, and concentrated. The residue was dissolved with stirring in MeOH (12 mL) at room temperature followed by the addition of K₂CO₃ (2 g). The final reaction mixture was stirred for 30 min, after which time it was diluted with EtOAc (20 mL) and water (10 mL). The layers were separated, and the organic layer was washed with brine (5 mL), dried over Na₂SO₄, filtered, and concentrated. The residue was purified over a Biotage Ultra SNAP silica gel column (25 g) eluting with a 10 CV gradient of 0 to 100% EtOAc in heptane to provide the intermediate alcohol (2.11 g, 6.54 mmol, 96%) (MW_{Calc}+Na=387.21; MW_{Obs}=387.36) after collection of the desired fractions, concentration and drying under vacuum.

To a stirred solution of (S,E)-5-(benzyloxy)-4-((tert-butyldimethylsilyl)oxy)pent-2-en-1-ol (2.100 g, 6.511 mmol) in THF (42.0 mL) at 5° C. was added triethylamine (1.180 ml, 8.465 mmol) followed by methanesulfonyl chloride (0.533 ml, 6.837 mmol). The reaction mixture was stirred for 30 min. Lithium triethylborohydride (26.0 ml, 26.045 mmol) was added to the completed initial reaction at room temperature, and the resultant reaction was stirred for 2 h. The completed intermediate reaction was diluted with MeOH (1 mL), then 0.1N HCl until obtain pH ˜8. The final mixture was extract with heptane (2×20 mL ea), and the combined organic layers were washed with water (5 mL), brine (5 mL), dried over Na₂SO₄, filtered, concentrated, and dried under vacuum to provide crude 129, which was used in the next reaction without further purification.

To a stirred solution of ethyl 2-oxoacetate (25 g, 122.442 mmol) and allyltrimethylsilane (3.89E+04 ml, 244.884 mmol) in DCM (500 mL) at 0° C. was added dropwise boron trifluoride etherate (30.8 mL, 244.884 mmol) over 30 min. The reaction mixture was warmed to room temperature and stirred for 16 h. The completed reaction was cooled to 0° C. followed by the slow addition of aq NaHCO₃ (500 mL). The layers were separated, and the organic layer was washed with NaHCO₃ (100 mL), and brine (50 mL). The organic layer was dried over Na₂SO₄, filtered over a pad of Celite (50 g), eluted with DCM (2×100 mL), and the combined filtrates concentrated. The crude product (ca. 17.6 g, 122.4 mmol, 100%) was used in the next reaction without further purification. To a stirred suspension of 20% sodium hydride (4.99 g, 124.853 mmol (previously titrated with heptane 3×10 mL ea)) in THF (200 mL) and DMF (200 mL) at 0° C. was added ethyl 2-hydroxypent-4-enoate (12 g, 83.235 mmol) over 5 min. The suspension was stirred for 15 min at 0° C., after which time (bromomethyl)benzene (17.80 g, 104.044 mmol) and then TBAI (3.07 g, 8.324 mmol) were added. The reaction mixture was warmed to room temperature and stirred for 3 h. The completed reaction was slowly quenched with NH₄Cl (100 mL) and extracted with a 1:1 ratio of EtOAc/MTBE (3×75 mL ea). The combined organic layers were washed with water (2×50 mL), brine (50 mL), dried over Na₂SO₄, filtered, concentrated, and purified over a Biotage Ultra SNAP silica gel column (340 g) eluting with a 10 CV gradient of 0 to 35% EtOAc in heptane to provide compounds 130 (16.0 g, 68.3 mmol, 82%) after collection of the desired fractions, concentration and drying under vacuum.

To a stirred solution of ethyl 2-(benzyloxy)pent-4-enoate (16 g, 68.29 mmol) in 1,4-dioxane (160 mL) and water (80 mL) at 0° C. was added 2,6-lutidine (19.89 ml, 170.725 mmol), osmium tetroxide (13.40 ml, 1.707 mmol) and sodium periodate (58.4 g, 273.16 mmol). The reaction was warmed to room temperature and stirred for 4 h. The completed reaction was diluted with water (100 mL) and extracted with DCM (4×75 mL ea). The combined organic layers were washed with 1 N HCl (75 mL), brine (50 mL), dried over Na₂SO₄, filtered, concentrated, and dried under vacuum to provide the crude aldehyde intermediate.

To a stirred solution of ethyl 2-(benzyloxy)-4-oxobutanoate (130, 17 g, 71.952 mmol) in benzene (500 mL) at room temperature was added ethylene glycol (20.06 ml, 359.762 mmol) and p-toluenesulfonic acid monohydrate (1.369 g, 7.195 mmol). The reaction mixture was warmed to reflux using a Dean-Stark apparatus to collect the water bi-product and stirred for 4 h. The completed reaction was cooled to room temperature and diluted with MTBE (100 mL). The layers were separated, and the organic layer was washed with aq NaHCO₃ (100 mL), brine (100 mL), and concentrated. The residue was diluted with a 1:1:1 ratio of MeOH:THF:water (50 mL ea) followed by the addition of lithium hydroxide (6.03 g, 251.834 mmol). The resulting reaction was stirred for 3 h, after which time it was quenched with aq NH₄CL (100 mL) and 1N HCl (5 mL) to obtain a pH of approx. 2-3. The layers were separated, the aqueous layer was extracted with EtOAc (4×50 mL ea), and the combined organic layers were washed with brine (2×50 mL ea). The organic layer was dried over Na₂SO₄, filtered, concentrated, and dried under vacuum to provide 131 (17.8 g, 70.6 mmol, 98%) (MW_{Calc}+H=253.10; MW_{Obs}=253.29) without further purification.

To a stirred solution of 2-(benzyloxy)-3-(1,3-dioxolan-2-yl)propanoic acid (131, 1.5 g, 5.946 mmol) in DCM (30 mL) at room temperature were added (S,E)-1-(benzyloxy)pent-3-en-2-ol (129, 1.143 g, 5.946 mmol), DMAP (0.145 g, 1.189 mmol), and then diisopropylcarbodiimide (1.019 ml, 6.541 mmol). The reaction mixture was stirred for 16 h, after which time it was diluted with MTBE (50 mL). The organic layer was washed with water (20 mL), NaHCO₃ (20 mL), and brine (20 mL), dried over Na₂SO₄, filtered, concentrated, and purified over a Biotage Ultra SNAP silica gel column (25 g) eluting with a 10 CV gradient of 0 to 50% EtOAc in heptane to provide compounds 132 (2.30 g, 5.39 mmol, 91%) (MW_{Calc}+Na=449.20; MW_{Obs}=449.35) as a mixture of diastereomers after collection of the desired fractions, concentration and drying under vacuum.

To a stirred solution of KHMDS (1.768 g, 8.863 mmol) in toluene (30 mL) at −78° C. was added (S,E)-1-(benzyloxy)pent-3-en-2-yl 2-(benzyloxy)-3-(1,3-dioxolan-2-yl)propanoate (132, 2.1 g, 4.924 mmol) in toluene (30 ml, 281.634 mmol) over 15 min maintaining the temperature below −65° C. The reaction was stirred for 10 min followed by the addition of TMS-C1 (1.133 ml, 8.863 mmol). The resulting reaction was stirred at −78° C. for 10 min, slowly warmed to room temperature over 2 h, and stirred for an additional 1 h. The completed reaction was quenched with water and 1N HCl (30 mL ea) and extracted with EtOAc (2×30 mL ea). The combined organic layers were washed with brine (20 mL), dried over Na₂SO₄, filtered, concentrated. The residue was dissolved in toluene (30 mL) and MeOH (5 mL), cooled to 0° C., followed by the addition of 2 M TMS-diazomethane (3.69 ml, 7.386 mmol) in MeOH. The final reaction was stirred for 10 min, after which time it was carefully concentrated and purified over a Biotage Ultra SNAP silica gel column (25 g) eluting with a 10 CV gradient of 0 to 40% EtOAc in heptane to provide compounds 133 (2.10 g, 4.77 mmol, 97%) (MW_{Calc}+Na=463.22; MW_{Obs}=463.50) as a 1:1 mixture of diastereomers after collection of the desired fractions, concentration and drying under vacuum.

To a stirred solution of methyl (2R,3R,E)-2-((1,3-dioxolan-2-yl)methyl)-2,6-bis(benzyloxy)-3-methylhex-4-enoate (133, 6.3 g, 14.301 mmol) in EtOAc (60 mL) and EtOH (60 mL) at room temperature was purged with a N₂ atmosphere (3×) followed by the addition of 10% Pd/C (0.8 g). The reaction was placed under an atmosphere of H₂ under balloon pressure and stirred for 48 h. The completed reaction was purged with a N₂ atmosphere (3×), diluted with EtOAc (20 mL), followed by the addition of Celite (10 g). The suspension was filtered over a pad of Celite (5 g), eluted with EtOAc (2×10 mL ea), and the filtrated was concentrated to a crude oil (3.4 g, 12.96 mmol, 91%) (MW_{Calc}+Na=285.14; MW_{Obs}=285.22), which was used in the next reaction without further purification.

To a stirred solution of methyl (2R,3R)-2-((1,3-dioxolan-2-yl)methyl)-2,6-dihydroxy-3-methylhexanoate (1.3 g, 4.956 mmol) in DMSO (15 ml, 211.381 mmol) at room temperature was added IBX (1.735 g, 6.195 mmol). The reaction mixture was stirred for 16 h, after which time it was slowly quenched with aq sodium thiosulfate (20 mL), followed by the addition of MTBE (20 mL). The layers were separated, and the organic layer was washed with water (2×10 mL ea), brine (10 mL), dried over Na₂SO₄, filtered, concentrated, and purified over a Biotage Ultra SNAP silica gel column (10 g) eluting with a 10 CV gradient of 0 to 50% EtOAc in heptane to provide compound 134 (1.01 g, 3.69 mmol, 78%) (MW_{Calc}+Na=283.13; MW_{Obs}=283.20) after collection of the desired fractions, concentration and drying under vacuum.

To a stirred solution of S-ethyl 2-(diethoxyphosphoryl)ethanethioate (3.23 g, 13.447 mmol) and lithium chloride (0.570 g, 13.447 mmol) in THF (20 mL) at room temperature was added triethylamine (1.66 mL, 11.91 mmol), and stirred for 5 min. The mixture was cooled to 0° C. followed by a dropwise addition of methyl (2R,3S)-2-((1,3-dioxolan-2-yl)methyl)-6-hydroxy-3-methyl-tetrahydro-2H-pyran-2-carboxylate (134, 1.0 g, 3.842 mmol) in THF (15 mL) maintaining the temperature below 5° C. The reaction mixture was warmed to room temperature and stirred for 16 h. The completed reaction was diluted with MTBE (20 mL), added silica gel (5 g), and filtered over silica gel (5 g) eluting with MTBE (3×10 mL ea). The filtrate was concentrated and purified over a Biotage Ultra SNAP silica gel column (25 g) eluting with a 10 CV gradient of 0 to 50% EtOAc in heptane to provide the α,β-unsaturated thioester intermediate (0.985 g, 2.84 mmol, 74%) after collection of the desired fractions, concentration and drying under vacuum.

To a stirred solution of methyl (2R,3S,E)-2-((1,3-dioxolan-2-yl)methyl)-8-(ethylthio)-2-hydroxy-3-methyl-8-oxooct-6-enoate (1.1 g, 3.175 mmol) in THF (22 mL) at room temperature was added 1 M potassium tert-butoxide (0.159 ml, 0.159 mmol). The reaction mixture was stirred for 30 min, after which time it was quenched with aq NH₄Cl. The mixture was extracted with MTBE (3×10 mL ea), and the combined organic layers were dried over Na₂SO₄, filtered, concentrated, and purified over a Biotage Ultra SNAP silica gel column (25 g) eluting with a 10 CV gradient of 0 to 50% EtOAc in heptane to provide compound 135 (802 mg, 2.315 mmol, 73%) (MW_{Calc}+Na=369.15; MW_{Obs}=369.56) after collection of the desired fractions, concentration and drying under vacuum.

To a stirred solution of methyl (2R,3S)-2-((1,3-dioxolan-2-yl)methyl)-6-(2-(ethylthio)-2-oxoethyl)-3-methyltetrahydro-2H-pyran-2-carboxylate (135, 610 mg, 1.761 mmol) in acetone (6.5 mL) was added 5% Pd—C(100 mg, 0.94 mmol) and then cooled to 0° C. Triethylsilane (0.844 mL, 5.282 mmol) was then added followed by warming to room temperature and stirring for 1 h. The completed reaction was diluted with EtOAc, Celite (5 g) was added, and then filtered over a pad Celite (5 g) eluting with EtOAc (3×5 mL ea). The combined filtrates were concentrated to provide the aldehyde (432 mg, 1.509 mmol, 86%) (MW_{Calc}+Na=309.17; MW_{Obs}=309.20) as an oil, which was used in next steps without further purification.

To a stirred solution of methyltriphenylphosphonium bromide (1290 mg, 3.611 mmol) in THF (10 mL) at 0° C. was added 1.6 M n-BuLi (2.052 mL, 3.283 mmol) dropwise followed by stirring for 15 min. The mixture was cooled to −78° C. after which time a solution of methyl (2R,3S)-2-((1,3-dioxolan-2-yl)methyl)-3-methyl-6-(2-oxoethyl)tetrahydro-2H-pyran-2-carboxylate (470 mg, 1.641 mmol) in THF (5 mL) was added dropwise. The reaction was warmed to room temperature, and then stirred for 2 h. The complete reaction was diluted with a 1:1 ratio of heptane:MTBE (30 mL) followed by the addition of silica gel (3 g), and then filtered over a pad of silica gel (10 g) eluting with MTBE (3×20 mL ea). The combined filtrates were concentrated to dryness, and the crude product (397 mg, 1.396 mmol, 85%) was used in the next step without further purification.

To a stirred solution of potassium carbonate (545 mg, 3.946 mmol), potassium ferricyanide (1299 mg, 3.946 mmol), hydroquinine 2,5-diphenyl-4,6-pyrimidinediyl diether (70.6 mg, 0.079 mmol) and potassium osmate(VI) dihydrate (9.69 mg, 0.026 mmol) in t-BuOH (1 mL) and water (1 mL) at room temperature for 10 min, and then cooled to 0° C. was added a solution of methyl (2R,3S)-2-((1,3-dioxolan-2-yl)methyl)-6-allyl-3-methyltetrahydro-2H-pyran-2-carboxylate (374 mg, 1.315 mmol) t-BuOH (0.2 mL) followed by water (0.2 mL). The reaction mixture was stirred at 0° C. for 4 h, after which time it was diluted with EtOAc (10 mL) followed by added aq sodium thiosulfate (10 mL). The layers were separated, and the organic layer was washed with aq NH₄CL (2×5 mL ea), brine (5 mL), dried over Na₂SO₄, filtered, concentrated, and purified over a Biotage Ultra SNAP silica gel column (4 g) eluting with a 10 CV gradient of 0 to 65% EtOAc in heptane to provide compound 136 (375 mg, 1.178 mmol, 90%) (MW_{Calc}+Na=341.17; MW_{Obs}=341.63) with a 3:1 ratio of isomers after collection of the desired fractions, concentration and drying under vacuum.

To a stirred solution of methyl (2R,3S)-2-((1,3-dioxolan-2-yl)methyl)-6-((S)-2,3-dihydroxypropyl)-3-methyltetrahydro-2H-pyran-2-carboxylate (373 mg, 1.172 mmol) in DCM (6 mL) at 0° C. was added pyridine (470 μL, 5.815 mmol) and a solution of p-toluenesulfonic anhydride (420 mg, 1.286 mmol) in DCM (4 mL). The reaction mixture was stirred at 0° C. for 1 h, warmed to room temperature, and stirred for 30 min. The completed reaction was diluted with EtOAc (10 mL), the layers separated, and the organic layer was washed with NaHCO₃ (5 mL), and brine (5 mL). The organic layer was filtered over a plug of silica gel (3 g), eluted with EtOAc (3×10 mL ea), and the combined filtrates were concentrated, and azeotroped to dryness with toluene (3×10 mL ea). The residue was dissolved with stirring in acetone (6 mL) and water (1 mL) followed by the addition of sodium azide (275 mg, 4.230 mmol). The final reaction mixture was warmed to 55° C. and stirred for 18 h. The completed reaction was cooled to room temperature, diluted with MBTE (20 mL), the layers separated, and the organic layer was washed with water (5 mL) and brine (5 mL). The organic layer was dried over Na₂SO₄, filtered, and concentrated to provide the crude azide intermediate (352 mg, 1.025 mmol, 87%) (MW_{Calc}+Na=366.17; MW_{Obs}=366.19), which was used in the next step without further purification.

To a stirred solution of methyl (2R,3S)-2-((1,3-dioxolan-2-yl)methyl)-6-((S)-3-azido-2-hydroxypropyl)-3-methyltetrahydro-2H-pyran-2-carboxylate (342 mg, 0.996 mmol) in a mixture of THF (5 mL) and water (1 mL) at room temperature was added 1 M trimethylphosphine in THF (1.992 mL, 1.992 mmol). The reaction mixture was stirred for 8 h, after which time it was concentrated, and azeotroped to dry with toluene (5×10 mL ea). The residue was dissolved with stirring in dimethylacetamide (3.5 mL) at room temperature followed by the addition of 7-methyl-1H-indole-5-carboxylic acid (209 mg, 1.195 mmol), HOBt (183 mg, 1.195 mmol), triethylamine (0.42 mL, 3.00 mmol), and then EDC (767 mg, 2.00 mmol). The reaction mixture was stirred for 19 h, after which time the completed reaction was diluted with water (5 mL) and EtOAc (10 mL). The layers were separated, and the organic layer was washed with water (5 mL) and brine (5 mL). The organic layer was dried over Na₂SO₄, filtered, concentrated, and purified over a Biotage Ultra SNAP silica gel column (10 g) eluting with a 10 CV gradient of 0 to 100% EtOAc in heptane to provide compound 137 (398 mg, 0.839 mmol, 84%) (MW_{Calc}+Na=497.24; MW_{Obs}=497.29) after collection of the desired fractions, concentration and drying under vacuum.

To a stirred solution of methyl (2R,3S)-2-((1,3-dioxolan-2-yl)methyl)-6-((S)-2-hydroxy-3-(7-methyl-1H-indole-5-carboxamido)propyl)-3-methyltetrahydro-2H-pyran-2-carboxylate (400 mg, 0.843 mmol) in pyridine (0.51 mL) at room temperature was added acetic anhydride (159 μl, 1.686 mmol). The reaction mixture was stirred for 20 min, after which time the completed reaction was diluted with EtOAc (10 mL), the layers separated, and the organic layer was washed with 1N HCl (5 mL), NH₄CL (5 mL), then brine (5 mL). The organic layer was dried over Na₂SO₄, filtered, concentrated, and purified over a Biotage Ultra SNAP silica gel column (10 g) eluting with a 10 CV gradient of 0 to 100% EtOAc in heptane to provide per-acylated intermediate (365 mg, 0.707 mmol, 84%) (MW_{Calc}+H=517.30; MW_{Obs}=517.48) after collection of the desired fractions, concentration and drying under vacuum.

To methyl (2R,3S)-2-((1,3-dioxolan-2-yl)methyl)-6-((S)-2-acetoxy-3-(7-methyl-1H-indole-5-carboxamido)propyl)-3-methyltetrahydro-2H-pyran-2-carboxylate (137, 150 mg, 0.29 mmol) at room temperature was added a previously degassed (N₂) solution of acetic acid (2.5 mL) and water (0.5 mL). The reaction vessel was sealed, warmed to 65° C. and stirred for 8 h. The completed reaction was cooled to room temperature, diluted with EtOAc (10 mL), the layers separated. The organic layer was washed with water (2×2 mL ea), brine (2 mL), concentrated, azeotroped with toluene (4×5 mL ea), and dried under vacuum. The resulting residue was dissolved with stirring in 1,2-DCE (2 mL) at room temperature followed by the addition of tert-butyl (S)-9-oxa-2,6-diazaspiro[4.5]decane-2-carboxylate (19, 106 mg, 0.436 mmol) and previously dried molecular sieves (350 mg). The suspension was stirred for 2 h, after which time added sodium triacetoxyborohydride (123 mg, 0.581 mmol) was added. The intermediate reaction mixture was stirred for 16 h, after which time it was quenched with aq NaHCO₃. The mixture was filtered over a plug of Celite (5 g), eluted with EtOAc (3×5 mL ea), and the layers separated. The organic layer was washed with brine (5 mL), dried over Na₂SO₄, filtered, concentrated, and purified over a Biotage Ultra SNAP silica gel column (4 g) eluting with a 10 CV gradient of 0 to 100% EtOAc in heptane, then 5 CV of 10% MeOH in DCM. The desired fractions were combined, concentrated, and dried under vacuum to provide the desired fully protected intermediate. The resultant residue was dissolved with stirring in MeOH (1 mL) and THF (1 mL) at room temperature followed by the addition of 1 N NaOH (1.0 mL, 1.00 mmol). The final reaction was warmed to 50° C. and stirred for 72 h. The completed reaction was cooled to room temperature, after which time it was neutralized with 1 N HCl (1.0 mL, 1.00 mmol), concentrated, and purified by HPLC to provide A-216 (9.4 mg, 0.015 mmol, 5%) (MW_{Calc}+H=643.37; MW_{Obs}=643.27), A-217 (13.8 mg, 0.021 mmol, 7%) (MW_{Calc}+H=643.37; MW_{Obs}=643.51), A-218 (4.7 mg, 0.007 mmol, 3%) (MW_{Calc}+H=643.37; MW_{Obs}=643.33), and A-219 (7.3 mg, 0.011 mmol, 4%) (MW_{Calc}+H=643.37; MW_{Obs}=643.48) after collection of the desired fractions, concentration and drying under vacuum.

### Preparation of A-220

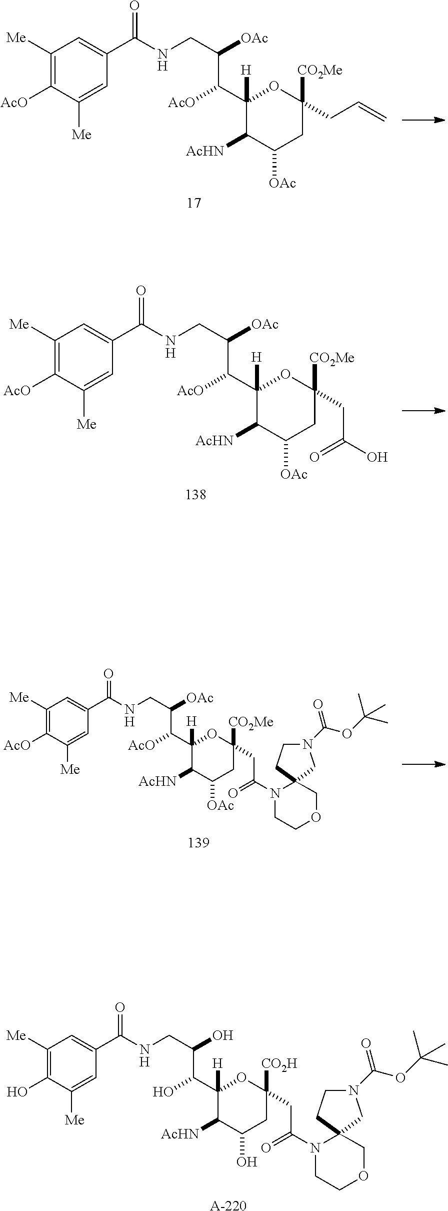

To a stirred suspension of (1R,2R)-1-((2R,3R,4S,6R)-3-acetamido-4-acetoxy-6-allyl-6-(methoxycarbonyl)tetrahydro-2H-pyran-2-yl)-3-(4-acetoxy-3,5-dimethylbenzamido)propane-1,2-diyl diacetate (17, 207 mg, 0.312 mmol) in acetone (4 mL) at room temperature was added sodium bicarbonate (105 mg, 1.25 mmol) and then potassium permanganate (197 mg, 1.25 mmol) followed by stirring for 16 h. The completed reaction was diluted with a mixture of MeOH (0.063 mL), 1 N HCl (1.25 mL, 1.249 mmol), pH 4 buffer solution (20 mL) and brine (10 mL) followed by adjusting the pH to 5-6 with an appropriate amount of 0.1 N HCl and stirred for an additional 5 min. The final mixture was extracted with 10% MeOH in EtOAc (6×10 mL ea), and the combined organic layers were dried over Na₂SO₄, filtered, and concentrated to a white solid. The solid was purified over a Biotage SNAP Ultra silica gel column (10 g) eluting with a gradient of 10%-20% MeOH (5 CV). The desired fractions were combined, concentrated, and dried under vacuum to provide compound 138 (150 mg, 0.220 mmol, 70%) (MW_{Calc}+H=681.24; MW_{Obs}=681.33).

To stirred solution of 2-((2S,4S,5R,6R)-5-acetamido-4-acetoxy-6-((1R,2R)-1,2-diacetoxy-3-(4-acetoxy-3,5-dimethylbenzamido)propyl)-2-(methoxycarbonyl)tetrahydro-2H-pyran-2-yl)acetic acid (138, 57 mg, 0.084 mmol) in DMF (3 mL) at room temperature was added tert-butyl (R)-9-oxa-2,6-diazaspiro[4.5]decane-2-carboxylate (19, 30.4 mg, 0.126 mmol, as the free amine) followed by HATU (47.8 mg, 0.126 mmol) and Hünig's base (0.073 ml, 0.419 mmol) followed by stirring for 72 h. The completed reaction was concentrated and placed directly onto a SNAP Ultra silica gel column (10 g) eluting with a gradient of 0% to 5% MeOH in EtOAc (10 CV) to provide compound 139 (30 mg, 0.033 mmol, 40%) (MW_{Calc}+H=905.40; MW_{Obs}=905.70).

To a stirred solution of (1R,2R)-1-((2R,3R,4S,6S)-3-acetamido-4-acetoxy-6-(2-((R)-2-(tert-butoxycarbonyl)-9-oxa-2,6-diazaspiro[4.5]decan-6-yl)-2-oxoethyl)-6-(methoxycarbonyl)tetrahydro-2H-pyran-2-yl)-3-(4-acetoxy-3,5-dimethylbenzamido)propane-1,2-diyl diacetate (53, 18.8 mg, 0.021 mmol) in MeOH (1 mL) at room temperature was added 1 N NaOH (0.208 mL, 0.208 mmol) followed by heating to 65° C. and stirring for 1 h. The completed reaction was cooled to room temperature, neutralized by addition of 1 N HCl (0.187 mL, 0.187 mmol) and concentrated to a residue. The residue was dissolved in MeOH, filtered through 2-micron syringe filter, and purified by reversed-phase HPLC to provide A-220 (6.3 mg, 0.009 mmol, 42%) (MW_{Calc}+H=723.34; MW_{Obs}=723.55).

### Preparation of A-221 and A-222

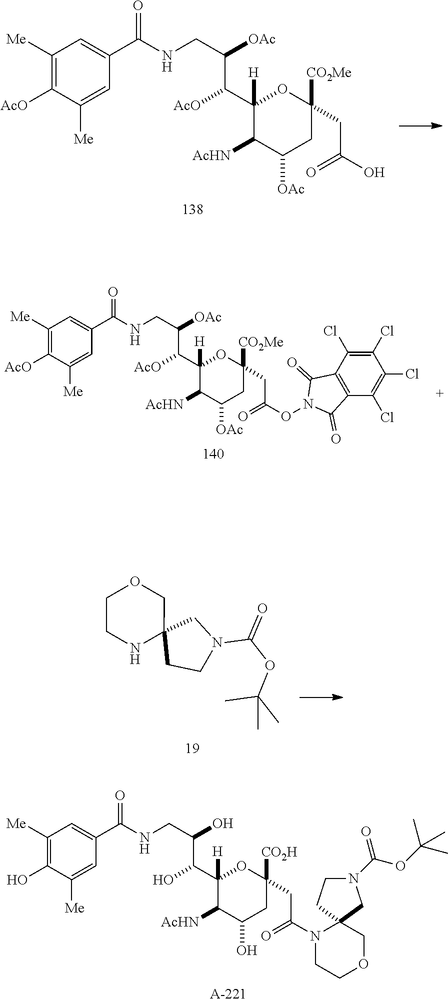

To a stirred suspension of 2-((2S,4S,5R,6R)-5-acetamido-4-acetoxy-6-((1R,2R)-1,2-diacetoxy-3-(4-acetoxy-3,5-dimethylbenzamido)propyl)-2-(methoxycarbonyl)tetrahydro-2H-pyran-2-yl) acetic acid (138, 150 mg, 0.22 mmol) in THF (5 mL) at room temperature was added 4,5,6,7-tetrachloro-2-hydroxyisoindoline-1,3-dione (133 mg, 0.441 mmol), DMAP (26.9 mg, 0.22 mmol) followed by DIC (0.137 mL, 0.882 mmol). The reaction mixture was stirred at room temperature for 72 h after which time it was filtered through Celite eluting EtOAc (3×20 mL ea). The filtrate was concentrated and then placed on a Biotage SNAP Ultra silica gel column (10 g) eluting with a gradient of 50% to 100% ethyl acetate in heptane, then re-purification (10 g) eluting with a gradient of 50% to 85% ethyl acetate in heptane provided compound 140 (131 mg, 0.136 mmol, 62% yield) (MW_{Calc}+H=964.10; MW_{Obs}=964.22) after combining the desired fractions, concentration, and drying under vacuum.

To a stirred solution of (1R,2R)-1-((2R,3R,44S,6S)-3-acetamido-4-acetoxy-6-(methoxycarbonyl)-6-(2-oxo-2-((4,5,6,7-tetrachloro-1,3-dioxoisoindolin-2-yl)oxy)ethyl)tetrahydro-2H-pyran-2-yl)-3-(4-acetoxy-3,5-dimethylbenzamido)propane-1,2-diyl diacetate (140, 20 mg, 0.021 mmol) in DCM (2 mL) at room temperature was added (S)-tert-butyl 9-oxa-2,6-diazaspiro[4.5]decane-2-carboxylate (19, 5.03 mg, 0.021 mmol) followed by TEA (0.029 mL, 0.208 mmol) and stirring for 1 h. HATU (79 mg, 0.208 mmol) was added and stirred final reaction mixture was stirred for 72 h at room temperature. The completed reaction mixture was filtered over a plug of silica gel (5 g) eluting with ethyl acetate (3×10 mL). The filtrate was concentrated to dryness and the resultant residue was used in the next step without further purification.

To a solution of (1R,2R)-1-((2R,3R,4S,6S)-3-acetamido-4-acetoxy-6-(2-((S)-2-(tert-butoxycarbonyl)-9-oxa-2,6-diazaspiro[4.5]decan-6-yl)-2-oxoethyl)-6-(methoxycarbonyl)tetrahydro-2H-pyran-2-yl)-3-(4-acetoxy-3,5-dimethylbenzamido)propane-1,2-diyl diacetate (ca. 18.8 mg, 0.021 mmol) in MeOH (0.00 mmol) at room temperature was added 1 N NaOH (0.20 mL, 0.20 mmol) followed by stirring for 16 h. The completed reaction was neutralized with 1 N HCl (0.15 ml, 0.15 mmol), concentrated to dryness followed dissolving the residue in MeOH (2 mL), filtering through a 2-micron syringe filter and purified over a reversed-phase HPLC column to provide after concentration of the desired fractions and vacuum to dryness to provide A-221 (2.5 mg, 0.021 mmol, 17%) (MW_{Calc}+H=723.34; MW_{Obs}=723.59).

A-222 was prepared in a similar fashion to A-221 starting with compound 52 (20.0 mg, 0.021 mmol) and commercially available (R)-tert-butyl 9-oxa-2,6-diazaspiro[4.5]decane-2-carboxylate (5.0 mg, 0.021 mmol) to provide after purification A-222 (3.0 mg, 0.004 mmol, 20% overall yield) (MW_{Calc}+H=723.34; MW_{Obs}=723.55) which is the same structure as A-220.

### Preparation of A-223

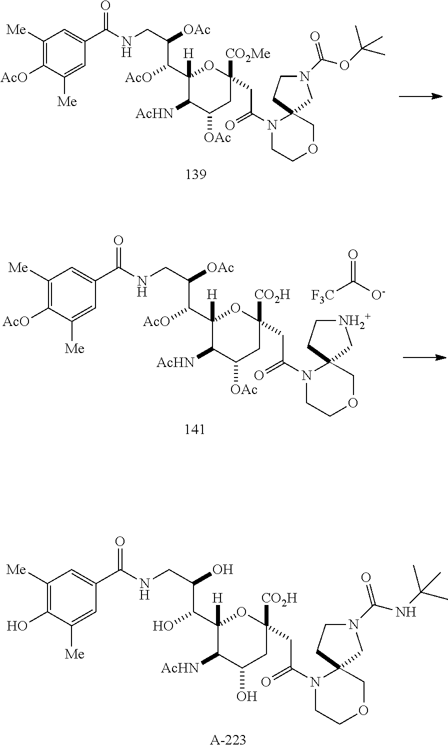

To (1R,2R)-1-((2R,3R,4S,6S)-3-acetamido-4-acetoxy-6-(2-((R)-2-(tert-butoxycarbonyl)-9-oxa-2,6-diazaspiro[4.5]decan-6-yl)-2-oxoethyl)-6-(methoxycarbonyl)tetrahydro-2H-pyran-2-yl)-3-(4-acetoxy-3,5-dimethylbenzamido)propane-1,2-diyl diacetate (139, 50 mg, 0.055 mmol) was added a 1:1 mixture of DCM (1 mL) to TFA (1 mL, 12.98 mmol) at room temperature followed by stirring for 20 min. The completed reaction was diluted with toluene (5 mL), concentrated, and azeotroped dryness with toluene (2×5 mL ea) to provide the crude salt product 141. (MW_{Calc}+H=805.35; MW_{Obs}=805.66).

To a stirred solution of (1R,2R)-1-((2R,3R,4S,6S)-3-acetamido-4-acetoxy-6-(methoxycarbonyl)-6-(2-oxo-2-((R)-9-oxa-2,6-diazaspiro[4.5]decan-6-yl)ethyl)tetrahydro-2H-pyran-2-yl)-3-(4-acetoxy-3,5-dimethylbenzamido)propane-1,2-diyl diacetate 2,2,2-trifluoroacetate (141, ca 50 mg, 0.054 mmol) in acetonitrile (2 mL) and TEA (0.1 mL, 0.717 mmol) at room temperature was added 2-isocyanato-2-methylpropane (10.8 mg, 0.109 mmol) followed by stirring for 30 min. The completed intermediate reaction was concentrated, dissolved in MeOH (1 mL, 24.718 mmol) and treated with 1 N NaOH (1.0 ml, 1.00 mmol) at room temperature, and stirred for 16 h. The completed reaction was neutralized by addition of HCl (0.9 mL, 0.90 mmol) and concentrated. The residue was sonicated in MeOH, filtered through 2-micron syringe filter, and purified two times over reversed-phase HPLC to provide A-223 (4.0 mg, 0.006 mmol, 23%) (MW_{Calc}+H=733.46; MW_{Obs}=733.60).

### Preparation of A-224 to A-227

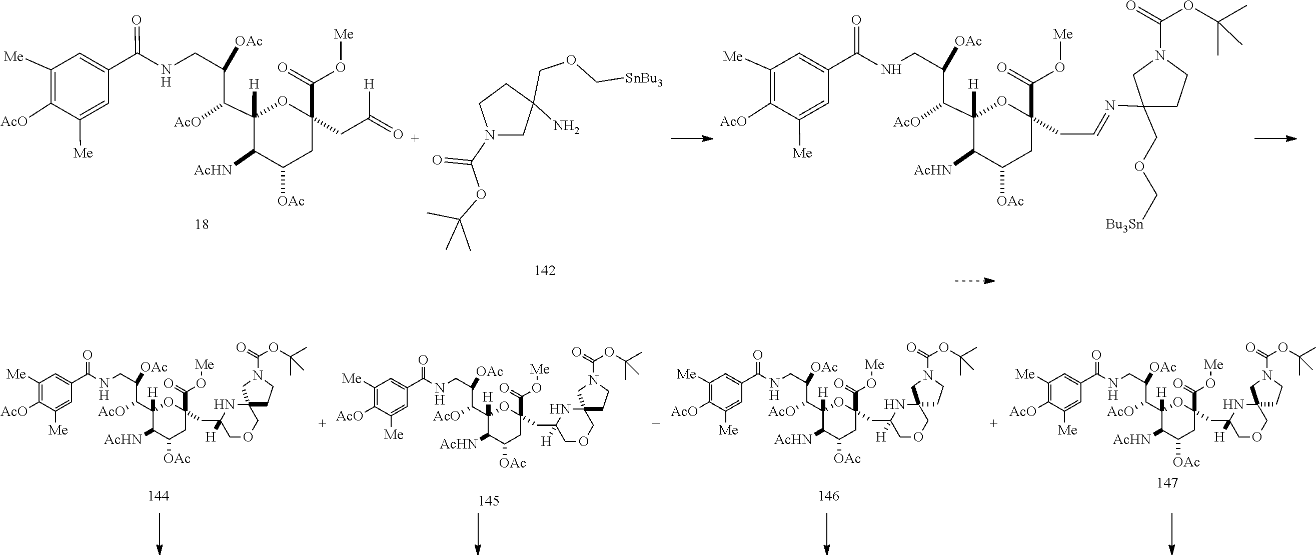

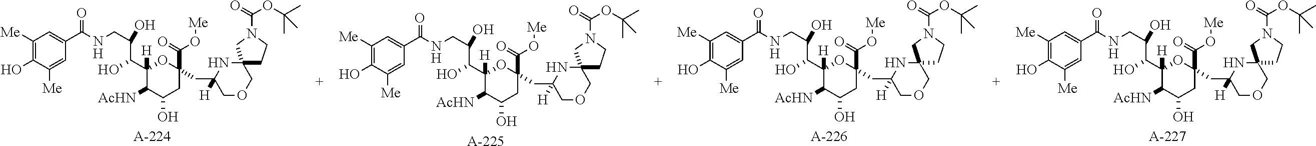

To a stirred solution of (1R,2R)-1-((2R,3R,4S,6S)-3-acetamido-4-acetoxy-6-(methoxycarbonyl)-6-(2-oxoethyl)tetrahydro-2H-pyran-2-yl)-3-(4-acetoxy-3,5-dimethylbenzamido)propane-1,2-diyl diacetate (18, 21 mg, 0.032 mmol) in benzene (1.050 mL) was added 4A MS (20 mg) followed by tert-butyl 3-amino-3-(((tributylstannyl)methoxy)methyl)pyrrolidine-1-carboxylate (142, 17.2 mg, 0.033 mmol). The mixture was stirred in an 80° C. for 16 h after which time it was cooled to room temperature, and then diluted with DCM (1 mL). The suspension was filtered through a pad of Celite, washed with DCM (3×2 mL ea), and the filtrate was concentrated in vacuo to obtain crude product 143, which was used directly in the next reaction. (TLC Rf: 0.24; 14:1 DCM:MeOH, Silica gel).

To a stirred suspension of copper(ii) trifluoromethanesulfonate (11.4 mg, 0.032 mmol) in hexafluoroisopropanol (0.5 mL, previously dried over 4A MS (100 mg/mL) for 2 h, then filtered) at room temperature under a N₂ atmosphere was added 2,6-lutidine (3.7 μL, 0.0.32 mmol) followed by stirring for 1 hour. (1R,2R)-1-((2R,3R,4S,6R)-3-acetamido-4-acetoxy-6-((E)-2-((-1-(tert-butoxycarbonyl)-3-(((tributylstannyl)methoxy)methyl)pyrrolidin-3-yl)imino)ethyl)-6-(methoxycarbonyl)tetrahydro-2H-pyran-2-yl)-3-(4-acetoxy-3,5-dimethylbenzamido)propane-1,2-diyl diacetate (143, 36.8 mg, 0.032 mmol) in DCM (0.5 mL) and dry hexafluoroisopropanol (1.0 mL) was then added into the blue suspension reagent mixture. The resulting green suspension was stirred at room temperature 16 h after which time I was concentrated, redissolved in a mixture of DCM (6 mL), sat. NaHCO₃ (7 mL) and 10% NH₄OH (3.0 mL), and stirred for an additional 10 min. The layers were separated, and the aqueous layer was extracted with DCM (3×10 mL ea). The combined organic layers were washed with 18% NaCl solution (10 mL), dried over Na₂SO₄, filtered, and the filtrate was concentrated. The residue was purified over a Biotage Ultra SNAP column (10 g) eluting with 1% MeOH in CH2Cl2 (3 CV) followed by a gradient of 1% to 7% MeOH in DCM. The desired fractions were combined, concentrated and dried under vacuum to provide a mixture of compounds 144-147 (13.0 mg, 0.0165 mmol, 47%) (MW_{Calc}+Na=899.40; MW_{Obs}=899.60). The mixture was separated using a chiral HPLC column to provide compound 144 (3.1 mg, 0.0035 mmol, 11%) (MW_{Calc}+H=877.40; MW_{Obs}=877.70), compound 145 (2.5 mg, 0.0029 mmol, 9%), (MW_{Calc}+H=877.40; MW_{Obs}=877.63) compound 146 (3.4 mg, 0.0039 mmol, 12%) (MW_{Calc}+H=877.40; MW_{Obs}=877.68), and compound 147 (2.6 mg, 0.0030 mmol, 9%) (MW_{Calc}+Na=899.40; MW_{Obs}=899.72).

To a stirred solution of (1R,2R)-1-((2R,3R,4S,6R)-3-acetamido-4-acetoxy-6-((2-(tert-butoxycarbonyl)-9-oxa-2,6-diazaspiro[4.5]decan-7-yl)methyl)-6-(methoxycarbonyl)tetrahydro-2H-pyran-2-yl)-3-(4-acetoxy-3,5-dimethylbenzamido)propane-1,2-diyl diacetate (146, 3 mg, 3.421 μmol) in MeOH (0.3 mL) and THF (0.3 mL) was added 1 N aqueous solution of NaOH (0.154 mL, 0.154 mmol) followed by stirring for 42 hours after which time it was neutralized/acidified with 1M HCl solution (0.12 mL) to pH 4 to 7. The crude solution was purified onto chiral HPLC column eluting with to provide A-226 (2.0 mg, 0.0029 mmol, 84%) (MW_{Calc}+H=695.35; MW_{Obs}=695.54).

Compounds 144, 145, and 147 were separately treated in a similar way to provide A-224 (0.5 mg, 0.0007 mmol, 20%) (MW_{Calc}+H=695.35; MW_{Obs}=695.42), A-225 (0.5 mg, 0.0007 mmol, 24%) (MW_{Calc}+H=695.35; MW_{Obs}=695.43), and A-227 (1.2 mg, 0.0017 mmol, 51%) (MW_{Calc}+H=695.35; MW_{Obs}=695.44).

### Alternative Preparation of A-226, A-227 and Preparation of A-228

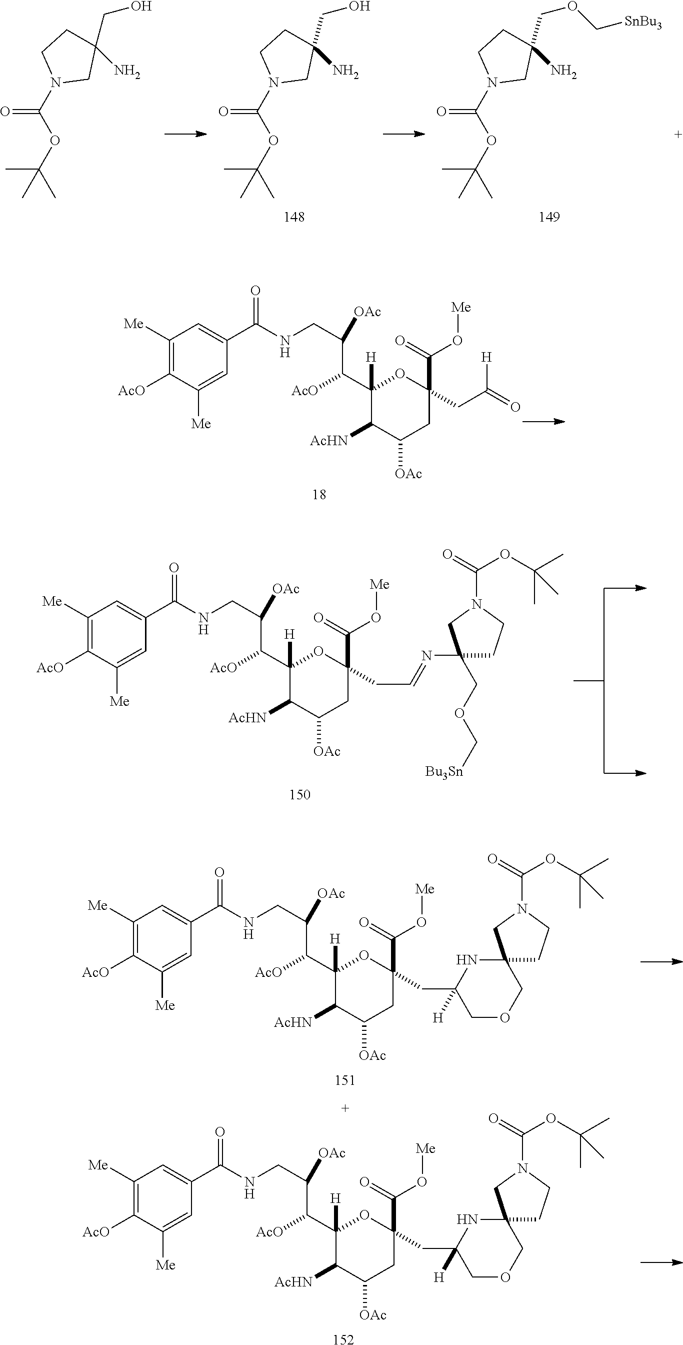

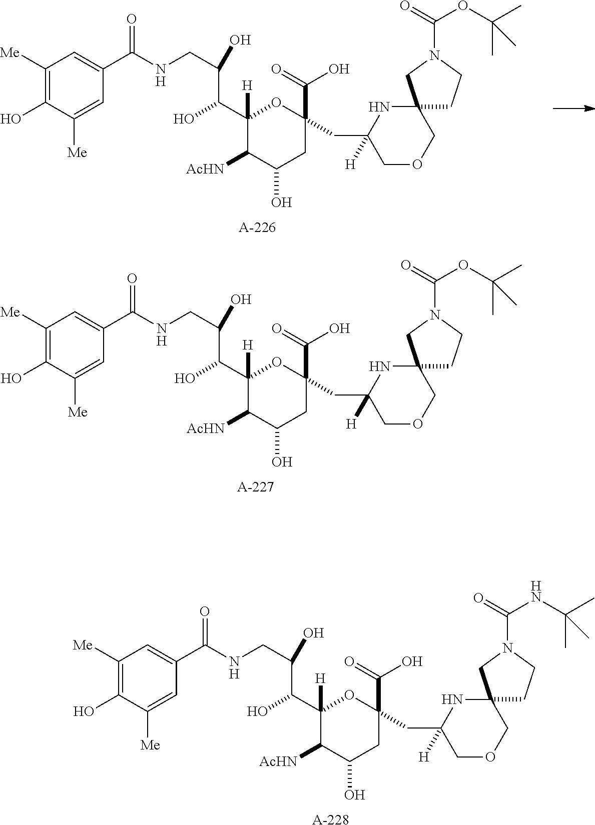

To a stirred solution of tert-butyl 3-amino-3-(hydroxymethyl)pyrrolidine-1-carboxylate (300 mg, 1.387 mmol) in 2-propanol (2.4 mL) at room temperature was added (2S,3S)-2,3-bis((4-methylbenzoyl)oxy)succinic acid (0.268 g, 0.694 mmol). The resultant mixture was stirred until it became mostly clear followed by warming to 65° C. and stirring for an additional hour to provide a white suspension. The mixture was cooled to ambient temperature followed by filtration of the white solid, washing the filter pad with 2-propanol (1 mL), drying under N₂ flow for 2 h, and in vacuum at 45° C. for 16 h to obtain 148 (247 mg, 0.60 mmol, 43%) as the (2S,3S)-2,3-bis((4-methylbenzoyl)oxy)succinate salt.

The chiral salt was rendered salt free by stirring in a solution of EtOAc (1.0 mL) at 0° C. followed by the addition of 6 N HCl (0.085 mL) and stirring for 1 h. The layers were separated, the aqueous layer extracted with EtOAc (1 mL), and the resultant aqueous layer was treated with 3 N NaOH (0.17 mL) followed by stirring for an additional 1 h. The resultant aqueous solution was lyophilized to dry, and the solid was suspended in EtOH (4 mL) followed by stirring stirred for 4 h at room temperature. The suspension was, filtered through a pad of Celite, the pad washed with EtOH (2 mL), and the filtrate was concentrated and dried under vacuum to provide 148 (295 mg, 1.364 mmol, 98%) (MW_{Calc}+Na=239.15; MW_{Obs}=239.31) as the free base.

To a stirred suspension of 60% sodium hydride (21.64 mg, 0.54 mmol, previously washed with cyclohexane (3×3 mL ea) in DMF (1.5 mL) under a N2 atmosphere at 0° C. was added dropwise tert-butyl (S)-3-amino-3-(hydroxymethyl)pyrrolidine-1-carboxylate (148, 90 mg, 0.416 mmol) in DMF (1 mL) over a 2-min period. The mixture was allowed to warm to room temperature and stirred for 30 min. after which time it was cooled to 0° C. followed by the dropwise addition of tributyl(iodomethyl)stannane (233 mg, 0.541 mmol) over a 2-min period. The final reaction mixture was slowly warmed to 50° C. and stirred for 16 h. The completed reaction was cooled to 0° C., and then quenched by adding sat. NH₄Cl (5 mL). The mixture was diluted with water (5 mL) and EtOAc (20 mL), and layers separated. The aqueous layer was extracted with EtOAc (20 mL), the combined organic layers were washed with brine (15 mL), and concentrated. The resultant residue was purified over a Biotage Ultra SNAP column (10 g) eluting with 1:1 heptane:DCM (3 CV), a gradient of 1:1 to 1:3 heptane:DCM (5 CV), a gradient of 1:1 to 1:3 heptane:EtOAc (5 CV), followed by 1:3 heptane:EtOAc (3 CV). The desired fractions were combined, concentrated, and dried under vacuum to provide compound 149 (79 mg, 0.152 mmol, 37%) (MW_{Calc}+Na=543.27; MW_{Obs}=543.33).

To a stirred solution of (1R,2R)-1-((2R,3R,4S,6S)-3-acetamido-4-acetoxy-6-(methoxycarbonyl)-6-(2-oxoethyl)tetrahydro-2H-pyran-2-yl)-3-(4-acetoxy-3,5-dimethylbenzamido)propane-1,2-diyl diacetate (18, 95 mg, 0.143 mmol) in toluene (0.760 mL) was added tert-butyl (S)-3-amino-3-(((tributylstannyl)methoxy)methyl)pyrrolidine-1-carboxylate (149, 74.2 mg, 0.143 mmol), and 4A MS (75 mg) followed by stirring at 85-90° C. for 16 h. The reaction was cooled to room temperature, diluted with DCM (1 mL), filtered through a pad of Celite, and the filter pad washed with DCM (4×1 mL ea). The combined filtrate was concentrated and dried under vacuum. The crude product, compound 150 was used in the next reaction without further purification.

To a stirred suspension of copper(ii) trifluoromethanesulfonate (52.7 mg, 0.146 mmol) in hexafluoroisopropanol (1.0 mL, previously dried over 4A MS (100 mg/mL) for 2 h, then filtered) at room temperature under a N₂ atmosphere was added 2,6-lutidine (16.98 μl, 0.146 mmol) followed by stirring for 1 hour. (1R,2R)-1-((2R,3R,4S,6R)-3-acetamido-4-acetoxy-6-((E)-2-(((S)-1-(tert-butoxycarbonyl)-3-(((tributylstannyl)methoxy)methyl)pyrrolidin-3-yl)imino)ethyl)-6-(methoxycarbonyl)tetrahydro-2H-pyran-2-yl)-3-(4-acetoxy-3,5-dimethylbenzamido)propane-1,2-diyl diacetate (150, 170 mg, 0.146 mmol) in DCM (1.0 mL) and dry hexafluoroisopropanol (2.0 mL) was then added into the blue suspension reagent mixture. The resulting green suspension was stirred at room temperature 16 h after which time I was concentrated, redissolved in a mixture of DCM (5 mL), sat. NaHCO₃ (2 mL) and 10% NH₄OH (0.5 mL), and stirred for an additional 10 min. The layers were separated, and the aqueous layer was extracted with DCM (3×10 mL ea). The combined organic layers were washed with 18% NaCl solution (10 mL), dried over Na₂SO₄, filtered, and the filtrate was concentrated. The residue was purified over a Biotage Ultra SNAP column (10 g) eluting with 1% MeOH in CH2Cl2 (3 CV) followed by a gradient of 1% to 7% MeOH in DCM. The desired fractions were combined, concentrated and dried under vacuum to provide a mixture of compounds 151 and 152 (61.5 mg, 0.070 mmol, 48%), which were separated by chiral HPLC ( ) eluting with 0 to provide compound 151 (18.5 mg, 0.021 mmol, 30%) (MW_{Calc}+H=877.40; MW_{Obs}=877.65), and compound 152 (37.0 mg, 0.042 mmol, 60%) (MW_{Calc}+H=877.40; MW_{Obs}=877.60), and a mixture of 151 and 152 (4.0 mg, 0.005 mmol, 7%).

To a stirred solution of (1R,2R)-1-((2R,3R,4S,6R)-3-acetamido-4-acetoxy-6-((2-(tert-butoxycarbonyl)-9-oxa-2,6-diazaspiro[4.5]decan-7-yl)methyl)-6-(methoxycarbonyl)tetrahydro-2H-pyran-2-yl)-3-(4-acetoxy-3,5-dimethylbenzamido)propane-1,2-diyl diacetate (151, 3 mg, 0.0034 mmol) in MeOH (300 μL) and THF (300 μL) was added 1N aqueous solution of NaOH (154 μL, 0.154 mmol) followed by stirring for 42 h. The completed reaction was neutralized/acidified with 1 M HCl solution (0.12 mL) to pH 4 to 7, the crude solution was purified directly using a chiral HPLC column to provide A-226 (2.0 mg, 0.003 mmol, 84%) (MW_{Calc}+H=695.35; MW_{Obs}=695.48).

Compound 152 (3 mg, 0.0034 mmol) was likewise treated under similar conditions described for compound 151 to provide A-227 (1.2 mg, 0.0017 mmol, 51%) (MW_{Calc}+H=695.35; MW_{Obs}=695.44).

To a stirred solution of (2R,4S,5R,6R)-5-acetamido-2-(((5S,7R)-2-(tert-butoxycarbonyl)-9-oxa-2,6-diazaspiro[4.5]decan-7-yl)methyl)-6-((1R,2R)-1,2-dihydroxy-3-(4-hydroxy-3,5-dimethylbenzamido)propyl)-4-hydroxytetrahydro-2H-pyran-2-carboxylic acid (A-226, 12 mg, 0.017 mmol) in CH2Cl2 (0.6 mL) was added TFA (0.06 mL, 0.78 mmol) at ambient temperature followed by stirring for 30 minutes. The completed reaction was concentrated and azeotroped to dryness with toluene 3×2 mL ea). The resultant residue was dissolved in acetonitrile (1 mL) followed by triethylamine (7.2 μl, 0.052 mmol) and MeOH (10.5 μl, 0.259 mmol) with stirring followed by the addition of 2-isocyanato-2-methylpropane (1.7 mg, 0.017 mmol) in acetonitrile (0.24 mL) at room temperature. The reaction was stirred for 2 h after which time the completed reaction was diluted with methanol (1.2 mL) and purified directly by HPLC to provide A-228 (11 mg, 0.016 mmol, 92%) (MW_{Calc}+H=694.36; MW_{Obs}=694.50).

### Preparation of A-229

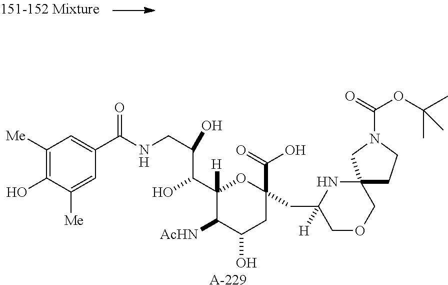

The mixture of 151 and 152 (4 mg, 0.0045 mmol) was treated under similar conditions described for compound 151 to provide a mixture of compounds or A-229 (1.5 mg, 0.0023 mmol, 51%) (MW_{Calc}+H=695.35; MW_{Obs}=695.40).

### Preparation of A-230 and A-231

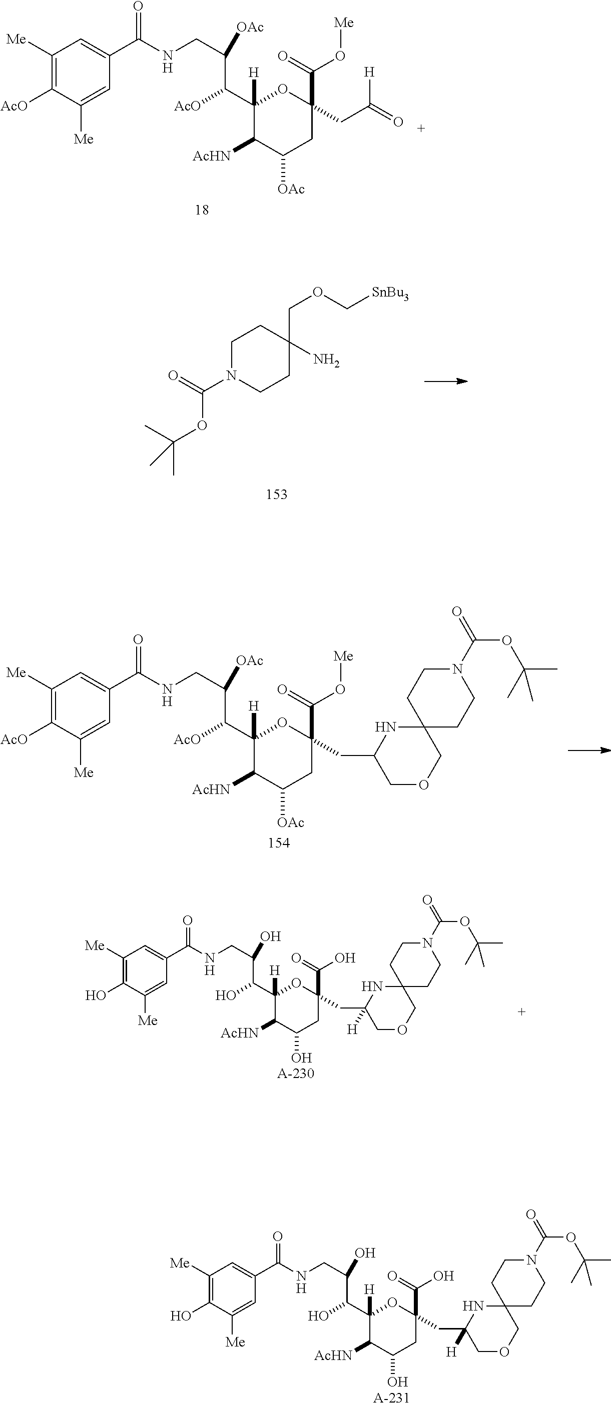

To a stirred mixture of (1R,2R)-1-((2R,3R,4S,6S)-3-acetamido-4-acetoxy-6-(methoxycarbonyl)-6-(2-oxoethyl)tetrahydro-2H-pyran-2-yl)-3-(4-acetoxy-3,5-dimethylbenzamido)propane-1,2-diyl diacetate (18, 25 mg, 0.038 mmol) was added commercially available tert-butyl 4-amino-4-(((tributylstannyl)-methoxy)methyl)piperidine-1-carboxylate (153, 20.06 mg, 0.038 mmol), and 4A MS (5 mg) in DCM (0.5 mL) at room temperature. The reaction mixture was stirred for 2 h after which time it was filtered through a pad of Celite, rinsed with DCM (3×2 mL ea), and the resultant filtrate was concentrated to dry.

In a separate flask 2,6-lutidine (4.38 μL, 0.038 mmol) and copper (II) trifluoromethanesulfonate (13.60 mg, 0.038 mmol) were combined in HFIP (0.4 mL) and stirred for one hour under a N₂ atms at room temperature. The concentrated intermediate (153) obtain from the previous step was dissolved in DCM (1.6 mL) and added to the reaction mixture followed by stirring for 16 h at room temperature. The completed reaction was quenched with 10% aqueous ammonium hydroxide (0.6 mL, 1.54 mmol) and stirred for an additional 10 minutes. The mixture was diluted with DCM (8 mL) and water (0.6 mL). The layers were separated, and the aqueous was extracted with DCM (2×5 mL ea). The combined organic layers were washed with 18% aqueous NaCl (5 mL), dried over MgSO4, filtered and concentrated to dry.

The resultant residue was purified over a Biotage Ultra SNAP column (10 g) eluting with 1% MeOH in DCM (3CV) followed by a gradient of 1%-8% MeOH in DCM (10 CV). The desired fractions were combined, concentrated and dried under vacuum to provide 154 (9.5 mg, 0.011 mmol, 28% yield) (MW_{Calc}+H=891.24; MW_{Obs}=891.42).

To a stirred solution of (1R,2R)-1-((2R,3R,4S,6R)-3-acetamido-4-acetoxy-6-((9-(tert-butoxycarbonyl)-4-oxa-1,9-diazaspiro[5.5]undecan-2-yl)methyl)-6-(methoxycarbonyl)tetrahydro-2H-pyran-2-yl)-3-(4-acetoxy-3,5-dimethylbenzamido)propane-1,2-diyl diacetate (154, 9 mg, 0.010 mol) in MeOH (0.36 mL) and THF (0.36 mL) was added 1 N aqueous NaOH (0.30 mL, 0.30 mmol) at room temperature followed by stirring for 72 h. The completed reaction was acidified with 2 M HCl solution (0.15 mL) to pH ˜4 followed by purification by HPLC followed concentration of the desired fractions and drying under vacuum to obtain A-230 (1.5 mg, 0.002 mmol, 21%) or and A-231 (3.0 mg, 0.004 mmol, 42%) (MW_{Calc}+H=709.36; MW_{Obs}=709.39).

### Preparation of A-232

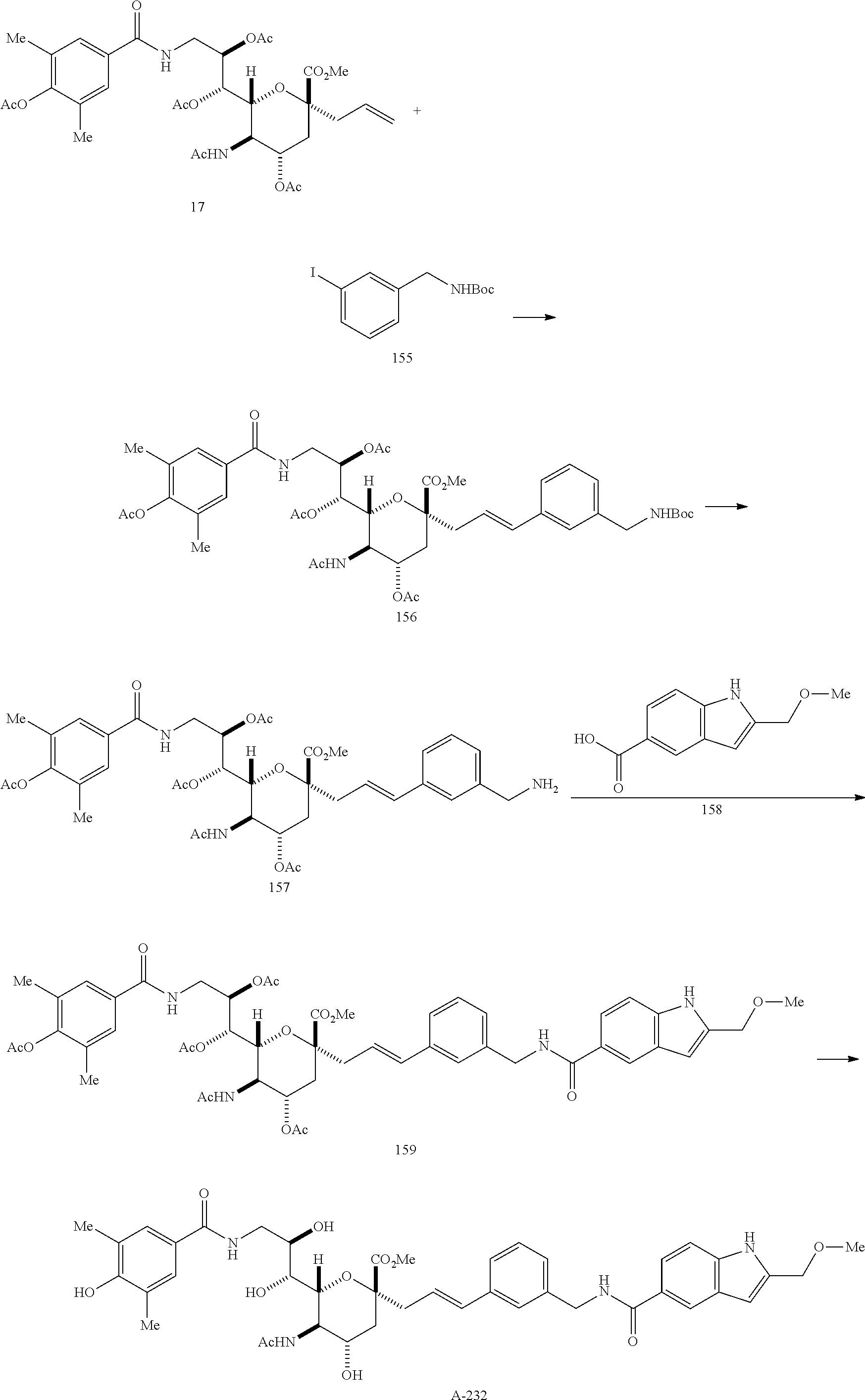

To a stirred solution of (1R,2R)-1-((2R,3R,4S,6R)-3-acetamido-4-acetoxy-6-allyl-6-(methoxycarbonyl)tetrahydro-2H-pyran-2-yl)-3-(4-acetoxy-3,5-dimethylbenzamido)propane-1,2-diyl diacetate (17, 0.30 g, 0.453 mmol) in acetonitrile (6.0 mL) was added tert-butyl 3-iodobenzylcarbamate (155, 0.302 g, 0.905 mmol) followed by N-cyclohexyl-N-methylcyclohexanamine (0.30 mL, 1.36 mmol). The resulting solution was degassed and charged with N2 (4×), followed by the addition of bis(tri-tert-butylphosphine)palladium(0) (23.1 mg, 0.045 mmol). The resulting reaction mixture was heated to 80-82° C. and stirred for 7 h after which time the completed reaction was cooled to room temperature, concentrated, and purified by Biotage SNAP silica gel (10 g) eluting with a gradient of 30-100% ethyl acetate in heptane (10 CV). The desired eluted fractions were concentrated and dried under vacuum to provide compound 156 (0.33 g, 0.38 mmol, 84%) (MW_{Calc}+H=868.38; MW_{Obs}=868.15).

To a stirred solution of (1R,2R)-1-((2R,3R,4S,6R)-3-acetamido-4-acetoxy-6-((E)-3-(3-(((tert-butoxycarbonyl)amino)-methyl)phenyl)allyl)-6-(methoxycarbonyl)tetrahydro-2H-pyran-2-yl)-3-(4-acetoxy-3,5-dimethylbenzamido)propane-1,2-diyl diacetate (156, 0.22 g, 0.253 mmol) in DCM (1.0 mL) was cooled to 0° C. followed by the slow addition of TFA (1.0 mL, 12.68 mmol). The reaction mixture was stirred at 0° C. for 40 mins after which time it was concentrated and azeotroped to dry with acetonitrile (2×10 mL ea). The crude product, 157, was used directly in the next reaction without further purification. (MW_{Calc}+H=768.33; MW_{Obs}=768.57).

To a stirred suspension of (1R,2R)-1-((2R,3R,4S,6R)-3-acetamido-4-acetoxy-6-((E)-3-(3-(aminomethyl)phenyl)allyl)-6-(methoxycarbonyl)tetrahydro-2H-pyran-2-yl)-3-(4-acetoxy-3,5-dimethylbenzamido)propane-1,2-diyl diacetate (157, 195 mg, 0.254 mmol), 2-(methoxymethyl)-1H-indole-5-carboxylic acid (158, 62.5 mg, 0.305 mmol) and HATU (193 mg, 0.508 mmol) in DCM (4.1 mL) at room temperature was added TEA (708 μl, 5.079 mmol) followed by stirring at room temperature for 16 h. The completed reaction was diluted with sat. NaHCO₃ (5 mL), extracted with ethyl acetate (2×10 mL ea). The combined organic layers were washed with brine (5 mL), dried over Na₂SO₄, filtered and concentrated to dry. The crude was submitted to analytical group for HPLC purification to provide compound 159 (147 mg, 0.154 mmol, 60%) (MW_{Calc}+H=955.39; MW_{Obs}=955.8).

To a solution of (1R,2R)-1-((2R,3R,4S,6R)-3-acetamido-4-acetoxy-6-(methoxycarbonyl)-6-((E)-3-(3-((2-(methoxymethyl)-1H-indole-5-carboxamido)methyl)phenyl)allyl)tetrahydro-2H-pyran-2-yl)-3-(4-acetoxy-3,5-dimethylbenzamido)propane-1,2-diyl diacetate (159, 147 mg, 0.154 mmol) in methanol (3.8 mL) at room temperature was added 1.0 N aqueous NaOH (2.62 mL, 2.62 mmol) followed by stirring for 36 h. The completed reaction was diluted with MeOH (2 mL) and injected directly onto a HPLC column to provide compound A-232 (56.0 mg, 0.072 mmol, 47%) (MW_{Calc}+H=773.34; MW_{Obs}=773.6) after collection of the desired fraction, concentration, and drying under vacuum.

### Preparation of A-233 to A-259

A-233 was prepared in a similar fashion to A-232 starting with compound 156 (16 mg, 0.018 mmol) and after Boc-deprotection using TFA in DCM, followed by condensing with tetrahydro-2H-pyran-3-carboxylic acid (7.2 mg, 0.055 mmol) with the resulting amine in the presence of HATU (10.5 mg, 0.028 mmol) and triethylamine (13 uL, 0.092 mmol) to provide A-233 (5.0 mg, 0.007 mmol, 40%) (MW_{Calc}+H=698.32; MW_{Obs}=698.30) after deacetylation, ester hydrolysis, HPLC purification, and rendering to dryness under vacuum.

A-234 was prepared in a similar fashion to A-232 starting with compound 157 (25 mg, 0.033 mmol) condensing commercially available 1H-indazole-5-carboxylic acid (7.0 mg, 0.043 mmol) in DMF (0.88 mL) in the presence of HATU (24.8 mg, 0.065 mmol) and triethylamine (23 uL, 0.163 mmol) to provide A-234 (8.3 mg, 0.009 mmol, 27%) (MW_{Calc}+H=730.30; MW_{Obs}=730.4) after deacetylation, ester hydrolysis, HPLC purification, and rendering to dryness under vacuum.

A-235 was prepared in a similar fashion to A-232 starting with compound 157 (25 mg, 0.033 mmol) condensing commercially available 1H-indazole-6-carboxylic acid (7.0 mg, 0.043 mmol) in DMF (0.88 mL) in the presence of HATU (24.8 mg, 0.065 mmol) and triethylamine (23 uL, 0.163 mmol) to provide A-235 (4.9 mg, 0.0067 mmol, 17%) (MW_{Calc}+H=730.30; MW_{Obs}=730.4) after deacetylation, ester hydrolysis, HPLC purification, and rendering to dryness under vacuum.

A-236 was prepared in a similar fashion to A-232 starting with compound 157 (25 mg, 0.033 mmol) condensing commercially available 1H-benzo[d]imidazole-5-carboxylic acid (7.0 mg, 0.043 mmol) in DMF (0.88 mL) in the presence of HATU (24.8 mg, 0.065 mmol) and triethylamine (23 uL, 0.163 mmol) to provide A-236 (3.1 mg, 0.0042 mmol, 11%) (MW_{Calc}+H=730.30; MW_{Obs}=730.4) after deacetylation, ester hydrolysis, HPLC purification, and rendering to dryness under vacuum.

A-237 was prepared in a similar fashion to A-232 starting with compound 157 (25 mg, 0.033 mmol) condensing commercially available 1H-benzo[d][1,2,3]triazole-6-carboxylic acid (7.0 mg, 0.043 mmol) in DMF (0.88 mL) in the presence of HATU (24.8 mg, 0.065 mmol) and triethylamine (23 uL, 0.163 mmol) to provide A-237 (5.3 mg, 0.0073 mmol, 19%) (MW_{Calc}+H=730.31; MW_{Obs}=731.4) after deacetylation, ester hydrolysis, HPLC purification, and rendering to dryness under vacuum.

A-238 was prepared in a similar fashion to A-232 starting with compound 157 (25 mg, 0.033 mmol) condensing commercially available 2,3-dimethyl-1H-indole-5-carboxylic acid (8.0 mg, 0.042 mmol) in DMF (0.88 mL) in the presence of HATU (24.8 mg, 0.065 mmol) and triethylamine (23 uL, 0.163 mmol) to provide A-238 (2.1 mg, 0.0027 mmol, 7.5%) (MW_{Calc}+H=757.35; MW_{Obs}=757.7) after deacetylation, ester hydrolysis, HPLC purification, and rendering to dryness under vacuum.

A-239 was prepared in a similar fashion to A-232 starting with compound 157 (25 mg, 0.033 mmol) condensing commercially available 4-(methylsulfonamido)benzoic acid (9.0 mg, 0.042 mmol) in DMF (0.88 mL) in the presence of HATU (24.8 mg, 0.065 mmol) and triethylamine (23 uL, 0.163 mmol) to provide A-239 (5.6 mg, 0.072 mmol, 19%) (MW_{Calc}+H=783.29; MW_{Obs}=783.4) after deacetylation, ester hydrolysis, HPLC purification, and rendering to dryness under vacuum.

A-240 was prepared in a similar fashion to A-232 starting with compound 157 (25 mg, 0.033 mmol) condensing commercially available 4-(pyrimidin-2-ylamino)benzoic acid (9.0 mg, 0.042 mmol) in DMF (0.88 mL) in the presence of HATU (24.8 mg, 0.065 mmol) and triethylamine (23 uL, 0.163 mmol) to provide A-240 (3.9 mg, 0.005 mmol, 13%) (MW_{Calc}+H=783.33; MW_{Obs}=783.4) after deacetylation, ester hydrolysis, HPLC purification, and rendering to dryness under vacuum.

A-241 was prepared in a similar fashion to A-232 starting with compound 157 (25 mg, 0.033 mmol) condensing commercially available 3-(thiazol-2-ylamino)benzoic acid (9.0 mg, 0.041 mmol) in DMF (0.88 mL) in the presence of HATU (24.8 mg, 0.065 mmol) and triethylamine (23 uL, 0.163 mmol) to provide A-241 (2.7 mg, 0.0034 mmol, 9%) (MW_{Calc}+H=788.29; MW_{Obs}=788.3) after deacetylation, ester hydrolysis, HPLC purification, and rendering to dryness under vacuum.

A-242 was prepared in a similar fashion to A-232 starting with compound 157 (25 mg, 0.033 mmol) condensing commercially available 1H-indole-5-carboxylic acid (7.0 mg, 0.043 mmol) in DMF (0.88 mL) in the presence of HATU (24.8 mg, 0.065 mmol) and triethylamine (23 uL, 0.163 mmol) to provide A-242 (1.1 mg, 0.0014 mmol, 3.7%) (MW_{Calc}+H=729.31; MW_{Obs}=729.4) after deacetylation, ester hydrolysis, HPLC purification, and rendering to dryness under vacuum.

A-243 was prepared in a similar fashion to A-232 starting with compound 157 (25 mg, 0.033 mmol) condensing commercially available 2-(1-hydroxyethyl)-1H-benzo[d]imidazole-5-carboxylic acid (9.0 mg, 0.044 mmol) in DMF (0.88 mL) in the presence of HATU (24.8 mg, 0.065 mmol) and triethylamine (23 uL, 0.163 mmol) to provide A-243 (2.6 mg, 0.0034 mmol, 8.9%) (MW_{Calc}+H=774.33; MW_{Obs}=774.4) after deacetylation, ester hydrolysis, HPLC purification, and rendering to dryness under vacuum.

A-244 was prepared in a similar fashion to A-232 starting with compound 157 (25 mg, 0.033 mmol) condensing commercially available 3-methyloxetane-3-carboxylic acid (5.0 mg, 0.043 mmol) in DMF (0.88 mL) in the presence of HATU (24.8 mg, 0.065 mmol) and triethylamine (23 uL, 0.163 mmol) to provide A-244 (0.8 mg, 0.0012 mmol, 3%) (MW_{Calc}+H=684.31; MW_{Obs}=684.4) after deacetylation, ester hydrolysis, HPLC purification, and rendering to dryness under vacuum.

A-245 was prepared in a similar fashion to A-232 starting with compound 157 (25 mg, 0.033 mmol) condensing commercially available 3-(((4-methylthiazol-2-yl)methyl)amino)benzoic acid (10.0 mg, 0.040 mmol) in DMF (0.88 mL) in the presence of HATU (24.8 mg, 0.065 mmol) and triethylamine (23 uL, 0.163 mmol) to provide A-245 (4.3 mg, 0.0053 mmol, 15%) (MW_{Calc}+H=816.32; MW_{Obs}=816.4) after deacetylation, ester hydrolysis, HPLC purification, and rendering to dryness under vacuum.

A-246 was prepared in a similar fashion to A-232 starting with compound 157 (25 mg, 0.033 mmol) condensing commercially available 3-((thiazol-5-ylmethyl)amino)benzoic acid (10.0 mg, 0.043 mmol) in DMF (0.88 mL) in the presence of HATU (24.8 mg, 0.065 mmol) and triethylamine (23 uL, 0.163 mmol) to provide A-246 (3.9 mg, 0.005 mmol, 14%) (MW_{Calc}+H=802.31; MW_{Obs}=802.4) after deacetylation, ester hydrolysis, HPLC purification, and rendering to dryness under vacuum.

A-247 was prepared in a similar fashion to A-232 starting with compound 157 (32 mg, 0.042 mmol) condensing commercially available 4-(pyrazin-2-ylamino)cyclohexanecarboxylic acid (12 mg, 0.054 mmol) in DMF (1.13 mL) in the presence of HATU (31.7 mg, 0.083 mmol) and triethylamine (29 uL, 0.163 mmol) to provide A-247 (12.8 mg, 0.016 mmol, 38%) (MW_{Calc}+H=789.38; MW_{Obs}=789.6) after deacetylation, ester hydrolysis, HPLC purification, and rendering to dryness under vacuum.

A-248 was prepared in a similar fashion to A-232 starting with compound 157 (32 mg, 0.042 mmol) condensing commercially available 2-(2-hydroxyethyl)-1H-benzo[d]imidazole-5-carboxylic acid (11.2 mg, 0.054 mmol) in DMF (1.13 mL) in the presence of HATU (31.7 mg, 0.083 mmol) and triethylamine (29 uL, 0.163 mmol) to provide A-248 (8.4 mg, 0.011 mmol, 26%) (MW_{Calc}+H=773.34; MW_{Obs}=774.6) after deacetylation, ester hydrolysis, HPLC purification, and rendering to dryness under vacuum.

A-249 was prepared in a similar fashion to A-232 starting with compound 157 (32 mg, 0.042 mmol) condensing commercially available 2-(1-hydroxy-2-methylpropan-2-yl)-1H-benzo[d]imidazole-5-carboxylic acid (11.2 mg, 0.054 mmol) in DMF (1.13 mL) in the presence of HATU (31.7 mg, 0.083 mmol) and triethylamine (29 uL, 0.163 mmol) to provide A-249 (13.2 mg, 0.016 mmol, 38%) (MW_{Calc}+H=802.37; MW_{Obs}=802.7) after deacetylation, ester hydrolysis, HPLC purification, and rendering to dryness under vacuum.

A-250 was prepared in a similar fashion to A-232 starting with compound 157 (32 mg, 0.042 mmol) condensing commercially available 2-(1H-indol-3-yl)-1H-benzo[d]imidazole-6-carboxylic acid (15.0 mg, 0.054 mmol) in DMF (1.13 mL) in the presence of HATU (31.7 mg, 0.083 mmol) and triethylamine (29 uL, 0.163 mmol) to provide A-250 (7.7 mg, 0.009 mmol, 21%) (MW_{Calc}+H=845.35; MW_{Obs}=845.7) after deacetylation, ester hydrolysis, HPLC purification, and rendering to dryness under vacuum.

A-251 was prepared in a similar fashion to A-232 starting with compound 157 (32 mg, 0.042 mmol) condensing commercially available 3-(tert-butylamino)benzoic acid (10.5 mg, 0.054 mmol) in DMF (1.13 mL) in the presence of HATU (31.7 mg, 0.083 mmol) and triethylamine (29 uL, 0.163 mmol) to provide A-251 (6.8 mg, 0.009 mmol, 21%) (MW_{Calc}+H=761.37; MW_{Obs}=761.6) after deacetylation, ester hydrolysis, HPLC purification, and rendering to dryness under vacuum.

A-252 was prepared in a similar fashion to A-232 starting with compound 157 (32 mg, 0.042 mmol) condensing commercially available 2-(1-(pyrimidin-2-yl)-1H-pyrrol-2-yl)-1H-benzo[d]imidazole-5-carboxylic acid (16.5 mg, 0.054 mmol) in DMF (1.13 mL) in the presence of HATU (31.7 mg, 0.083 mmol) and triethylamine (29 uL, 0.163 mmol) to provide A-252 (7.3 mg, 0.0084 mmol, 20%) (MW_{Calc}+H=873.35; MW_{Obs}=873.7) after deacetylation, ester hydrolysis, HPLC purification, and rendering to dryness under vacuum.

A-253 was prepared in a similar fashion to A-232 starting with compound 157 (32 mg, 0.042 mmol) condensing commercially available 2-(1-(pyrimidin-2-yl)-1H-pyrrol-2-yl)-1H-benzo[d]imidazole-5-carboxylic acid (16.5 mg, 0.054 mmol) in DMF (1.13 mL) in the presence of HATU (31.7 mg, 0.083 mmol) and triethylamine (29 uL, 0.163 mmol) to provide A-253 (12.4 mg, 0.014 mmol, 33%) (MW_{Calc}+H=789.38; MW_{Obs}=789.6) after deacetylation, ester hydrolysis, HPLC purification, and rendering to dryness under vacuum.

A-254 was prepared in a similar fashion to A-232 starting with compound 157 (32 mg, 0.042 mmol) condensing commercially available 2-methyl-1-oxo-2,3,4,9-tetrahydro-1H-pyrido[3,4-b]indole-6-carboxylic acid (13.2 mg, 0.054 mmol) in DMF (1.13 mL) in the presence of HATU (31.7 mg, 0.083 mmol) and triethylamine (29 uL, 0.163 mmol) to provide A-254 (10.3 mg, 0.012 mmol, 29%) (MW_{Calc}+H=812.35; MW_{Obs}=812.7) after deacetylation, ester hydrolysis, HPLC purification, and rendering to dryness under vacuum.

A-255 was prepared in a similar fashion to A-232 starting with compound 157 (32 mg, 0.042 mmol) condensing commercially available (1R,3S)-3-(pyrazin-2-ylamino)cyclopentane-carboxylic acid (11.2 mg, 0.054 mmol) in DMF (1.13 mL) in the presence of HATU (31.7 mg, 0.083 mmol) and triethylamine (29 uL, 0.163 mmol) to provide A-255 (8.6 mg, 0.011 mmol, 26%) (MW_{Calc}+H=775.36; MW_{Obs}=775.6) after deacetylation, ester hydrolysis, HPLC purification, and rendering to dryness under vacuum.

A-256 was prepared in a similar fashion to A-232 starting with compound 157 (32 mg, 0.042 mmol) condensing commercially available (1R,3S)-3-(pyrimidin-2-ylamino)cyclohexanecarboxylic acid (13.2 mg, 0.054 mmol) in DMF (1.13 mL) in the presence of HATU (31.7 mg, 0.083 mmol) and triethylamine (29 uL, 0.163 mmol) to provide A-256 (13.4 mg, 0.017 mmol, 40%) (MW_{Calc}+H=789.38; MW_{Obs}=789.7) after deacetylation, ester hydrolysis, HPLC purification, and rendering to dryness under vacuum.

A-257 was prepared in a similar fashion to A-232 starting with compound 157 (26 mg, 0.034 mmol) condensing commercially available 4-(tert-butylamino)benzoic acid (8.5 mg, 0.044 mmol) in DMF (0.92 mL) in the presence of HATU (25.8 mg, 0.083 mmol) and triethylamine (24 uL, 0.068 mmol) to provide A-257 (0.9 mg, 0.001 mmol, 4%) (MW_{Calc}+H=761.37; MW_{Obs}=761.6) after deacetylation, ester hydrolysis, HPLC purification, and rendering to dryness under vacuum.

A-258 was prepared in a similar fashion to A-232 starting with compound 157 (26 mg, 0.034 mmol) condensing commercially available (1S,4S)-4-((5-carbamoylpyridin-2-yl)amino)-cyclohexanecarboxylic acid (11.6 mg, 0.044 mmol) in DMF (0.92 mL) in the presence of HATU (25.8 mg, 0.083 mmol) and triethylamine (24 uL, 0.068 mmol) to provide A-258 (12.6 mg, 15 mmol,58%) (MW_{Calc}+H=831.39; MW_{Obs}=831.7) after deacetylation, ester hydrolysis, HPLC purification, and rendering to dryness under vacuum.

A-259 was prepared in a similar fashion to A-232 starting with compound 157 (26 mg, 0.034 mmol) condensing commercially available (1S,3R)-3-((5-methylpyridin-2-yl)amino)cyclohexanecarboxylic acid (10.3 mg, 0.044 mmol) in DMF (0.92 mL) in the presence of HATU (25.8 mg, 0.083 mmol) and triethylamine (24 uL, 0.068 mmol) to provide A-259(9.7 mg, 0.01 mmol, 35%) (MW_{Calc}+H=802.40; MW_{Obs}=802.6) after deacetylation, ester hydrolysis, HPLC purification, and rendering to dryness under vacuum.

### Preparation of A-260

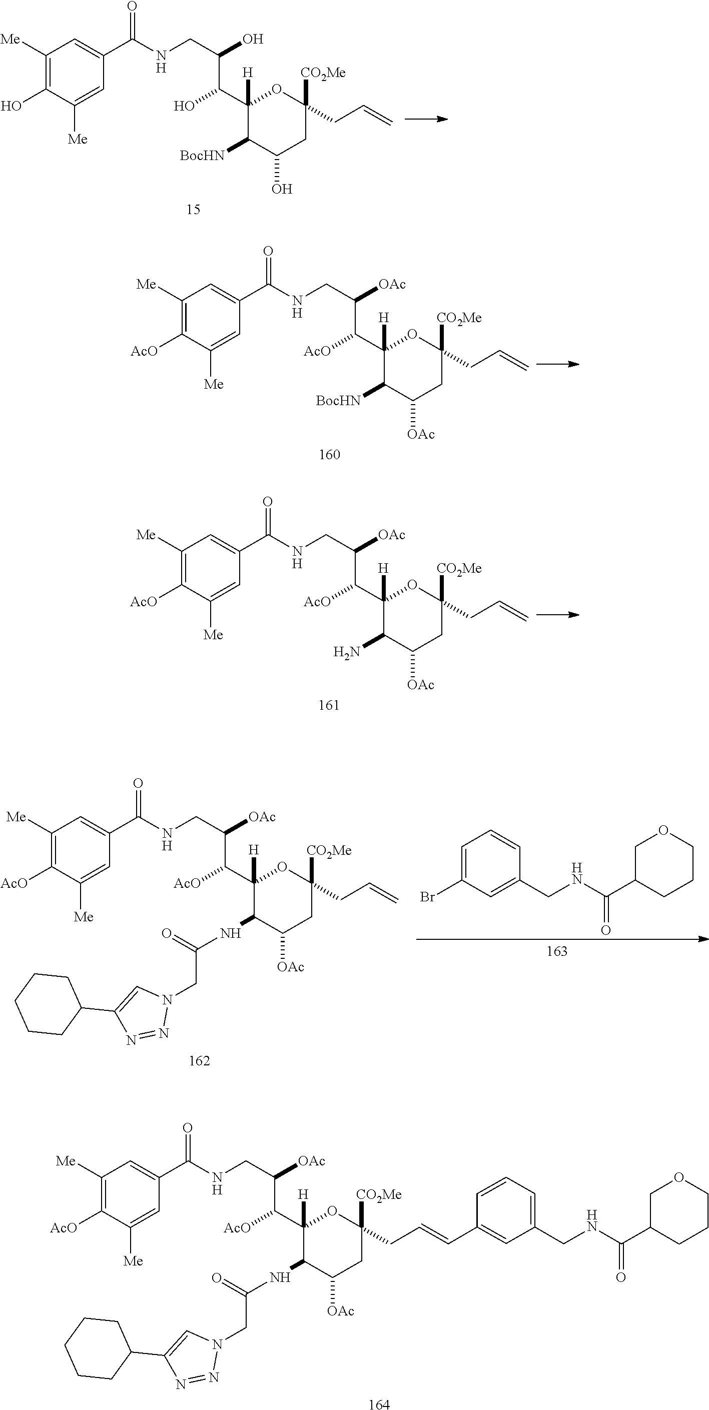

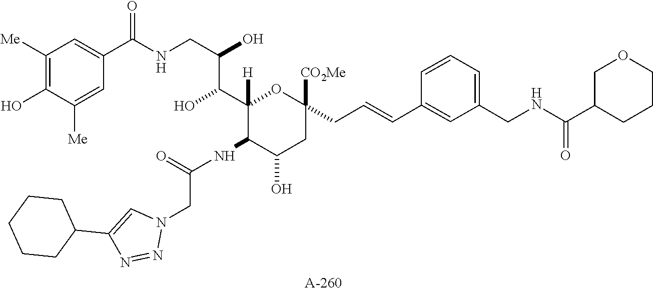

To a stirred solution of (2R,4S,5R,6R)-methyl 2-allyl-5-((tert-butoxycarbonyl)amino)-6-((1R,2R)-1,2-dihydroxy-3-(4-hydroxy-3,5-dimethylbenzamido)propyl)-4-hydroxytetrahydro-2H-pyran-2-carboxylate (15, 190 mg, 0.344 mmol) in pyridine (0.95 mL) at room temperature was added acetic anhydride (0.95 mL, 10.07 mmol) followed by stirring for 18 hours total monitoring by LCMS. The completed reaction was diluted with ethyl acetate (10 mL) and washed with 1N HCl (5 mL), sat NH₄Cl (5 mL), and brine (5 mL). The organic layer was dried over Na₂SO₄, filtered, concentrated, and azeotroped with toluene (3×10 mL ea) followed by drying under vacuum to provide crude 160.

To a stirred solution of (2R,4S,5R,6R)-2-allyl-5-((tert-butoxycarbonyl)amino)-6-((1R,2R)-1,2-dihydroxy-3-(4-hydroxy-3,5-dimethylbenzamido)propyl)-4-hydroxytetrahydro-2H-pyran-2-carboxylic acid (160, ca. 127 mg, 0.344 mmol) in DCM (0.95 mL) was added dropwise TFA (0.95 mL, 12.33 mmol) followed by stirring for an additional 8 minutes. The completed reaction was diluted with 3 mL of toluene and evaporated under vacuum. The residue was azeotroped with toluene (3×3 mL) to dryness to provide 161 to be used in the next step. (MW_{Calc}+H=621.26; MW_{Obs}=621.41).

To a stirred solution of (1R,2R)-3-(4-acetoxy-3,5-dimethylbenzamido)-1-((2R,3R,4S,6R)-4-acetoxy-6-allyl-3-amino-6-(methoxycarbonyl)tetrahydro-2H-pyran-2-yl)propane-1,2-diyl diacetate (161, ca. 213 mg, 0.344 mmol) in acetonitrile (2.85 mL) was added 2-(4-cyclohexyl-1H-1,2,3-triazol-1-yl)acetic acid (108 mg, 0.516 mmol) followed by triethylamine (0.192 mL, 1.375 mmol) and then immediately after HATU (261 mg, 0.688 mmol). The reaction was stirred at room temperature for 30 min after which time the reaction was diluted with water (5 mL) and extracted with EtOAc (2×5 mL ea). The organic layer was washed with brine (5 mL), dried over Na₂SO₄, filtered, concentrated, and placed under vacuum to dryness. The resulting oil was purified over a Biotage SNAP silica gel column (10 g) eluting with 0-100% EtOAc in heptane to provide desired amide 162 (266 mg, 0.328 mmol, 95% from 15) (MW_{Calc}+H=812.36; MW_{Obs}=812.61) as a solid after concentration of the desired fractions and drying under vacuum.

A vial of (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 50 mg, 0.062 mmol) and (1 N-(3-iodobenzyl)tetrahydro-2H-pyran-3-carboxamide (163, 42.5 mg, 0.123 mmol) was purged under stream of N₂ for 30 min after which time bis(tri-tert-butylphosphine)palladium (0) (3.15 mg, 6.2 μmol) in MeCN (1.2 mL) followed by methyl dicyclohexylamine (39.6 μl, 0.185 mmol). The resultant reaction mixture was warmed with stirring to 80° C. for 3 h, and then purified directly onto a Biotage SNAP silica gel column (10 g) eluting with a gradient of 0-100% ethyl acetate in heptane (10 CV). The fractions containing product were evaporated, and azeotroped to dry with methanol (2×10 mL ea) to provide crude 164 that was used in the subsequent step. (MW_{Calc}+H=1029.47; MW_{Obs}=1029.42).

To a stirred solution of (1R,2R)-3-(4-acetoxy-3,5-dimethylbenzamido)-1-((2R,3R,4S,6R)-4-acetoxy-3-(2-(4-cyclohexyl-1H-1,2,3-triazol-1-yl)acetamido)-6-(methoxycarbonyl)-6-((E)-3-(3-((tetrahydro-2H-pyran-3-carboxamido)methyl)phenyl)allyl)tetrahydro-2H-pyran-2-yl)propane-1,2-diyl diacetate (164, ca. 63.4 mg, 0.062 mmol) in methanol (1 mL) at room temperature was added 1N sodium hydroxide (0.62 mL, 0.62 mmol) followed by stirring for 26 h. The completed reaction was diluted with methanol and injected in aliquots on a HPLC column to provide (A-260) (12.0 mg, 0.014 mmol, 23%) (MW_{Calc}+H=847.42; MW_{Obs}=847.5).

### Preparation of A-261

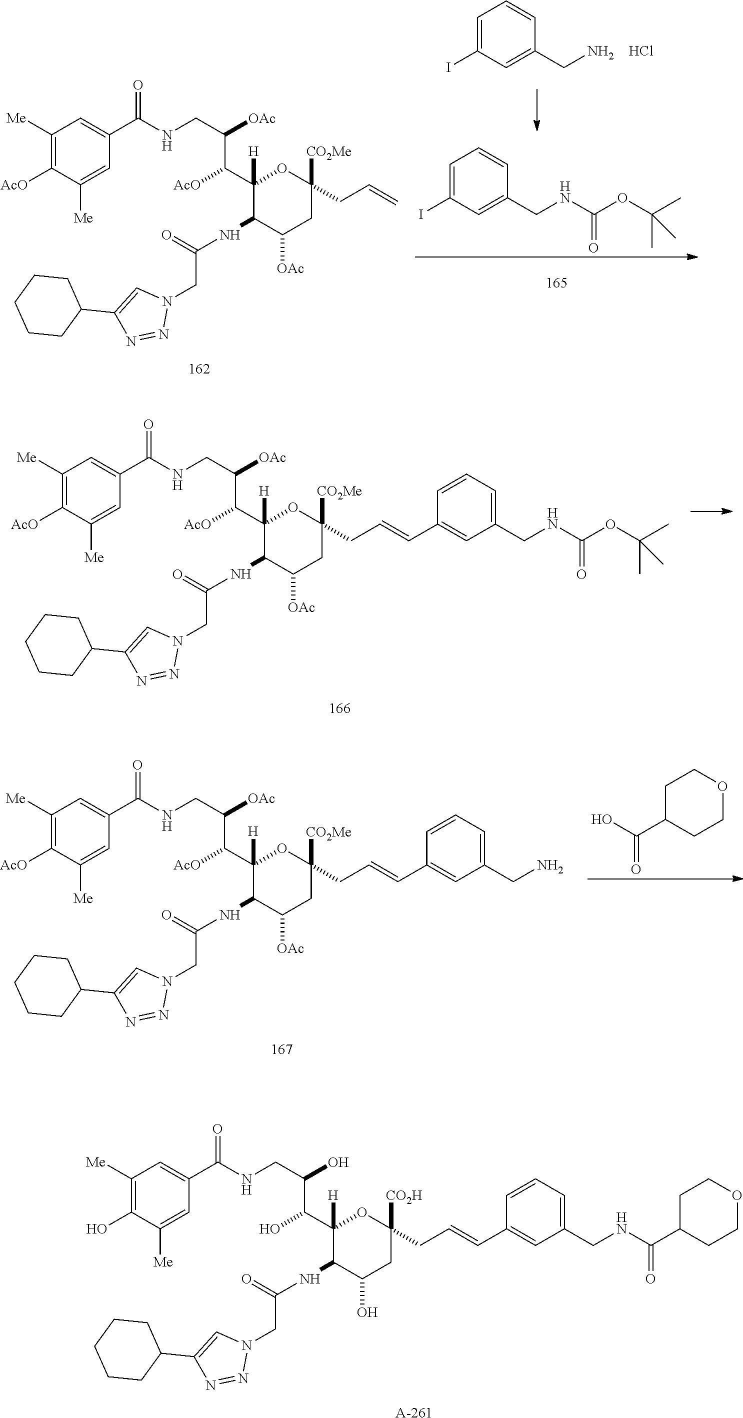

To a stirred solution of 3-iodobenzylamine hydrochloride (516 mg, 1.915 mmol) in THF (6.192 mL) at room temperature was added triethylamine (0.8 mL, 5.744 mmol) and BOC-anhydride (0.60 mL, 2.585 mmol). The reaction mixture was stirred for 1 h, after which time EtOAc (20 mL) was added, and the organic layer was washed with water (5 mL) brine (5 mL), dried over Na₂SO₄, filtered, and concentrated to dry. The crude residue was purified over a Biotage Ultra SNAP silica gel column (25 g) eluting with 10 CV gradient of 0 to 50% EtOAc in heptane to provide tert-butyl 3-iodobenzylcarbamate 165 (554 mg, 1.663 mmol, 87% yield) as a crystalline solid after concentration of the desired fractions and drying under vacuum.

To a stirred solution of (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 90 mg, 0.111 mmol) in MeCN (1.5 mL) was added tert-butyl 3-iodobenzylcarbamate (165, 73.9 mg, 0.222 mmol), and methyl dicyclohexylamine (71.2 μl, 0.333 mmol). The mixture was purged under a N₂ atmosphere for 10 min followed by the addition of bis(tri-tert-butylphosphine)palladium (5.67 mg, 0.011 mmol), warming to 80° C., and stirring for 4.5 h. The mixture was then heated in closed vial at 80° C. for 4.5 hours. The completed reaction was cooled to room temperature followed by applying directly to a Biotage Ultra SNAP silica gel column (10 g) eluting with a 10 CV gradient of 0 to 100% EtOAc in heptane to provide compound 166 (100 mg, 0.098 mmol, 89%) (MW_{Calc}+H=1017.47; MW_{Obs}=1017.72) as a clear film after concentration of the desired fractions and drying under vacuum.

To a stirred solution of (1R,2R)-3-(4-acetoxy-3,5-dimethylbenzamido)-1-((2R,3R,4S,6R)-4-acetoxy-6-((E)-3-(3-(((tert-butoxycarbonyl)amino)methyl)phenyl)allyl)-3-(2-(4-cyclohexyl-1H-1,2,3-triazol-1-yl)acetamido)-6-(methoxycarbonyl)tetrahydro-2H-pyran-2-yl)propane-1,2-diyl diacetate (166. 20.0 mg, 0.020 mmol) in dichloromethane (0.5 mL) was added trifluoroacetic acid (0.5 mL) followed by stirring for 10 minutes. The completed reaction was concentrated, azeotroped to dry with toluene (3×2 mL ea), and dried under high vacuum to provide crude 167, which was used in the next step without purification. (MW_{Calc}+H=917.42; MW_{Obs}=917.64).

To a stirred solution of (1R,2R)-3-(4-acetoxy-3,5-dimethylbenzamido)-1-((2R,3R,4S,6R)-4-acetoxy-6-((E)-3-(3-(((tert-butoxycarbonyl)amino)methyl)phenyl)allyl)-3-(2-(4-cyclohexyl-1H-1,2,3-triazol-1-yl)acetamido)-6-(methoxycarbonyl)tetrahydro-2H-pyran-2-yl)propane-1,2-diyl diacetate (167, 20 mg, 0.020 mmol) in MeCN (0.7 mL) at room temperature was added triethylamine (11 uL, 0.079 mmol), and tetrahydro-pyran-4-carboxylic acid (5.12 mg, 0.256 mmol), followed by HATU (15 mg, 0.039 mmol). The reaction was stirred for 4 h, after which time water (3 mL) and EtOAc (3 mL) were added to the completed reaction. The layers were separated, and the aqueous layer was extracted with EtOAc (3 mL), the combined organic layers were concentrated, and azeotroped to dry with methanol (2×3 mL ea). The crude residue was dissolved in MeOH followed by the addition of 1 N aq. NaOH (0.35 mL, 0.35 mmol), and stirred for 24 h at room temperature. The completed hydrolysis was neutralized with 1N HCl (0.35 mL, 0.35 mmol) followed by purification directly by HPLC to provide A-261 (6.2 mg, 0.007 mmol, 37%) (MW_{Calc}+H=847.42; MW_{Obs}=847.7) after concentration of the desired fractions and drying under vacuum.

### Preparation of A-262 to A-282

A-262 was prepared in a similar fashion to A-261 starting with compound 167 (20 mg, 0.020 mmol) and commercially available 3-chlorobenzoic acid (6.2 mg, 0.039 mmol) in MeCN (0.7 mL) with triethylamine (11 uL, 0.079 mmol) and HATU (15 mg, 0.039 mmol) to provide A-262 (6.1 mg, 0.007 mmol, 35%) (MW_{Calc}+H=873.35; MW_{Obs}=873.6) after deacetylation, ester hydrolysis, HPLC purification, and rendering to dryness under vacuum.

A-263 was prepared in a similar fashion to A-261 starting with compound 167 (20 mg, 0.020 mmol) and commercially available 4-chlorobenzoic acid (6.2 mg, 0.039 mmol) in MeCN (0.7 mL) with triethylamine (11 uL, 0.079 mmol) and HATU (15 mg, 0.039 mmol) to provide A-263(6.0 mg, 0.007 mmol, 34%) (MW_{Calc}+H=873.35; MW_{Obs}=873.6) after deacetylation, ester hydrolysis, HPLC purification, and rendering to dryness under vacuum.

A-264 was prepared in a similar fashion to A-261 starting with compound 167 (19 mg, 0.019 mmol) and commercially available cyclopentanecarboxylic acid (3.4 mg, 0.030 mmol) in MeCN (0.7 mL) with diisopropylethylamine (15 uL, 0.084 mmol) and HATU (11.4 mg, 0.030 mmol) to provide A-264 (2.5 mg, 0.003 mmol, 16%) (MW_{Calc}+H=831.42; MW_{Obs}=831.6) after deacetylation, ester hydrolysis, HPLC purification, and rendering to dryness under vacuum.

A-265 was prepared in a similar fashion to A-261 starting with compound 167 (19 mg, 0.019 mmol) and commercially available 3-methoxypropionic acid (3.1 mg, 0.030 mmol) in MeCN (0.7 mL) with diisopropylethylamine (15 uL, 0.084 mmol) and HATU (11.4 mg, 0.030 mmol) to provide A-265 (5.8 mg, 0.007 mmol, 37%) (MW_{Calc}+H=821.40; MW_{Obs}=821.6) after deacetylation, ester hydrolysis, HPLC purification, and rendering to dryness under vacuum.

A-266 was prepared in a similar fashion to A-261 starting with compound 167 (19 mg, 0.019 mmol) and commercially available 1-acetyl-4-piperidinecarboxylic acid (5.1 mg, 0.030 mmol) in MeCN (0.7 mL) with diisopropylethylamine (15 uL, 0.084 mmol) and HATU (11.4 mg, 0.030 mmol) to provide A-266 (6.1 mg, 0.007 mmol, 36%) (MW_{Calc}+H=888.45; MW_{Obs}=888.7) after deacetylation, ester hydrolysis, HPLC purification, and rendering to dryness under vacuum.

A-267 was prepared in a similar fashion to A-261 starting with compound 167 (19 mg, 0.019 mmol) and commercially available 2-tetrahydrofuroic acid (3.5 mg, 0.030 mmol) in MeCN (0.7 mL) with diisopropylethylamine (15 uL, 0.084 mmol) and HATU (11.4 mg, 0.030 mmol) to provide A-267 (3.2 mg, 0.004 mmol, 20%) (MW_{Calc}+H=833.41; MW_{Obs}=833.6) after deacetylation, ester hydrolysis, HPLC purification, and rendering to dryness under vacuum.

A-268 was prepared in a similar fashion to A-261 starting with compound 167 (19 mg, 0.019 mmol) and commercially available tetrahydro-2H-pyran-3-carboxylic acid (3.9 mg, 0.030 mmol) in MeCN (0.7 mL) with diisopropylethylamine (15 uL, 0.084 mmol) and HATU (11.4 mg, 0.030 mmol) to provide A-268 (5.0 mg, 0.006 mmol, 31%) (MW_{Calc}+H=847.42; MW_{Obs}=847.6) after deacetylation, ester hydrolysis, HPLC purification, and rendering to dryness under vacuum.

A-269 was prepared in a similar fashion to A-261 starting with compound 167 (19 mg, 0.019 mmol) and commercially available α,α,α-trifluoro-o-toluic acid (5.7 mg, 0.030 mmol) in MeCN (0.7 mL) with triethylamine (11 uL, 0.079 mmol) and HATU (11.4 mg, 0.030 mmol) to provide A-269 (4.6 mg, 0.005 mmol, 27%) (MW_{Calc}+H=907.38; MW_{Obs}=097.7) after deacetylation, ester hydrolysis, HPLC purification, and rendering to dryness under vacuum.

A-270 was prepared in a similar fashion to A-261 starting with compound 167 (19 mg, 0.019 mmol) and commercially available 2-pyrazinecarboxylic acid (3.7 mg, 0.030 mmol) in MeCN (0.7 mL) with triethylamine (11 uL, 0.079 mmol) and HATU (11.4 mg, 0.030 mmol) to provide A-270 (2.2 mg, 0.003 mmol, 14%) (MW_{Calc}+H=841.38; MW_{Obs}=841.6) after deacetylation, ester hydrolysis, HPLC purification, and rendering to dryness under vacuum.

A-271 was prepared in a similar fashion to A-261 starting with compound 167 (19 mg, 0.019 mmol) and commercially available 3-Isoxazolecarboxylic acid (3.4 mg, 0.030 mmol) in MeCN (0.7 mL) with triethylamine (11 uL, 0.079 mmol) and HATU (11.4 mg, 0.030 mmol) to provide A-271 (1.2 mg, 0.001 mmol, 8%) (MW_{Calc}+H=830.37; MW_{Obs}=830.5) after deacetylation, ester hydrolysis, HPLC purification, and rendering to dryness under vacuum.

A-272 was prepared in a similar fashion to A-261 starting with compound 167 (20 mg, 0.020 mmol) and commercially available 2-oxabicyclo[4.1.0]heptane-7-carboxylic acid (4.2 mg, 0.029 mmol) in MeCN (0.7 mL) with diisopropylethylamine (15 uL, 0.088 mmol) and HATU (11.2 mg, 0.030 mmol) to provide A-272 (5.4 mg, 0.006 mmol, 31%) (MW_{Calc}+H=859.42; MW_{Obs}=849.4) after deacetylation, ester hydrolysis, HPLC purification, and rendering to dryness under vacuum.

A-273 was prepared in a similar fashion to A-261 starting with compound 167 (20 mg, 0.020 mmol) and commercially available 1-methyl-2-oxo-1,2-dihydropyridine-4-carboxylic acid (4.5 mg, 0.029 mmol) in MeCN (0.7 mL) with diisopropylethylamine (15 uL, 0.08 mmol) and HATU (11.2 mg, 0.030 mmol) to provide A-271 (7.6 mg, 0.009 mmol, 44%) (MW_{Calc}+H=870.40; MW_{Obs}=870.33) after deacetylation, ester hydrolysis, HPLC purification, and rendering to dryness under vacuum.

A-274 was prepared in a similar fashion to A-261 starting with compound 167 (20 mg, 0.020 mmol) and commercially available tetrahydropyran-4-acetic acid (4.3 mg, 0.029 mmol) in MeCN (0.7 mL) with diisopropylethylamine (15 uL, 0.08 mmol) and HATU (11.2 mg, 0.030 mmol) to provide A-274 (6.3 mg, 0.007 mmol, 34%) (MW_{Calc}+H=861.44; MW_{Obs}=861.4) after deacetylation, ester hydrolysis, HPLC purification, and rendering to dryness under vacuum.

A-275 was prepared in a similar fashion to A-261 starting with compound 167 (20 mg, 0.020 mmol) and commercially available 1-(pyridin-2-yl)-1H-pyrazole-4-carboxylic acid (5.6 mg, 0.029 mmol) in MeCN (0.7 mL) with diisopropylethylamine (15 uL, 0.08 mmol) and HATU (11.2 mg, 0.030 mmol) to provide A-275 (3.7 mg, 0.004 mmol, 20%) (MW_{Calc}+H=907.41; MW_{Obs}=907.4) after deacetylation, ester hydrolysis, HPLC purification, and rendering to dryness under vacuum.

A-276 was prepared in a similar fashion to A-261 starting with compound 167 (30 mg, 0.033 mmol) and commercially available 4-methylthiazole-5-carboxylic acid (14.1 mg, 0.098 mmol) in DCM (0.53 mL) with triethylamine (91 uL, 0.654 mmol) and HATU (24.9 mg, 0.065 mmol) to provide A-276 (4.7 mg, 0.005 mmol, 17%) (MW_{Calc}+H=860.36; MW_{Obs}=860.6) after deacetylation, ester hydrolysis, HPLC purification, and rendering to dryness under vacuum.

A-277 was prepared in a similar fashion to A-261 starting with compound 167 (30 mg, 0.033 mmol) and commercially available 1H-pyrazole-4-carboxylic acid (11 mg, 0.098 mmol) in DCM (0.53 mL) with triethylamine (91 uL, 0.654 mmol) and HATU (24.9 mg, 0.065 mmol) to provide A-277 (2.2 mg, 0.003 mmol, 8%) (MW_{Calc}+H=829.38; MW_{Obs}=829.5) after deacetylation, ester hydrolysis, HPLC purification, and rendering to dryness under vacuum.

A-278 was prepared in a similar fashion to A-261 starting with compound 167 (25 mg, 0.027 mmol) and commercially available 1H-indole-5-carboxylic acid (5.3 mg, 0.033 mmol) in DCM (0.44 mL) with triethylamine (76 uL, 0.545 mmol) and HATU (20.7 mg, 0.055 mmol) to provide A-278 (2.3 mg, 0.003 mmol, 10%) (MW_{Calc}+H=878.40; MW_{Obs}=878.5) after deacetylation, ester hydrolysis, HPLC purification, and rendering to dryness under vacuum.

A-279 was prepared in a similar fashion to A-261 starting with compound 167 (25 mg, 0.027 mmol) and commercially available 4-hydroxy-3-methoxybenzoic acid (5.5 mg, 0.033 mmol) in DCM (0.44 mL) with triethylamine (76 uL, 0.545 mmol) and HATU (20.7 mg, 0.055 mmol) to provide A-279 (0.9 mg, 0.001 mmol, 4%) (MW_{Calc}+H=885.40; MW_{Obs}=885.6) after deacetylation, ester hydrolysis, HPLC purification, and rendering to dryness under vacuum.

A-280 was prepared in a similar fashion to A-261 starting with compound 167 (25 mg, 0.027 mmol) and commercially available 1H-benzo[d]imidazole-5-carboxylic acid (5.3 mg, 0.033 mmol) in DCM (0.44 mL) with triethylamine (76 uL, 0.545 mmol) and HATU (20.7 mg, 0.055 mmol) to provide A-280 (2.3 mg, 0.003 mmol, 10%) (MW_{Calc}+H=879.40; MW_{Obs}=879.6) after deacetylation, ester hydrolysis, HPLC purification, and rendering to dryness under vacuum.

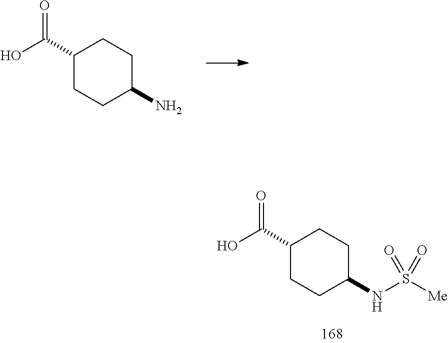 [SMILES: N[C@H]1CC[C@H](C(=O)O)CC1 ; CS(=O)(=O)N[C@H]1CC[C@H](C(=O)O)CC1]

To a stirred solution of (1R,4R)-4-aminocyclohexanecarboxylic acid (311.3 mg, 2.174 mmol) in THF (6.24 mL) at 0° C. was added 1 N aqueous NaOH (5.44 mL, 5.44 mmol), and stirred for 15 min, after which time methanesulfonyl chloride (541 μL, 6.957 mmol) was added. The reaction was warmed to room temperature, and stirred for 16 h after which time water (5 mL) was added. The quenched mixture was extracted with EtOAc (3×5 mL ea), and the combined organic layers were washed with brine (5 mL), dried over MgSO₄, filtered and concentrated to dryness. The crude product 168 was used in the next reaction without further purification.

A-281 was prepared in a similar fashion to A-261 starting with compound 167 (30 mg, 0.033 mmol) and (1R,4R)-4-(methylsulfonamido)cyclohexanecarboxylic acid 168, 8.7 mg, 0.039 mmol) in DCM (0.53 mL) with triethylamine (91 uL, 0.654 mmol) and HATU (25 mg, 0.030 mmol) to provide A-281 (8.3 mg, 0.009 mmol, 28%) (MW_{Calc}+H=938.43; MW_{Obs}=938.7) after deacetylation, ester hydrolysis, HPLC purification, and rendering to dryness under vacuum.

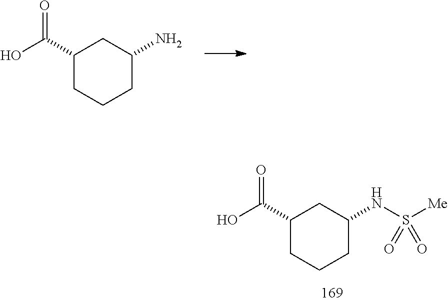 [SMILES: N[C@@H]1CCC[C@H](C(=O)O)C1 ; CS(=O)(=O)N[C@@H]1CCC[C@H](C(=O)O)C1]

Compound 169 was prepared in a similar fashion to 168 starting with (1S,3R)-3-aminocyclo-hexanecarboxylic acid 0, 307.8 mg, 2.15 mmol) and methanesulfonyl chloride (535 μl, 6.879 mmol) to provide crude 169, which was used in the next reaction without further purification.

A-282 was prepared in a similar fashion to A-261 starting with compound 167 (30 mg, 0.033 mmol) and (1S,3R)-3-(methylsulfonamido)cyclohexanecarboxylic acid (169, 8.7 mg, 0.039 mmol) in DCM (0.53 mL) with triethylamine (91 uL, 0.654 mmol) and HATU (25 mg, 0.030 mmol) to provide A-282 (8.5 mg, 0.009 mmol, 29%) (MW_{Calc}+H=938.43; MW_{Obs}=938.6) after deacetylation, ester hydrolysis, HPLC purification, and rendering to dryness under vacuum.

### Preparation of A-283

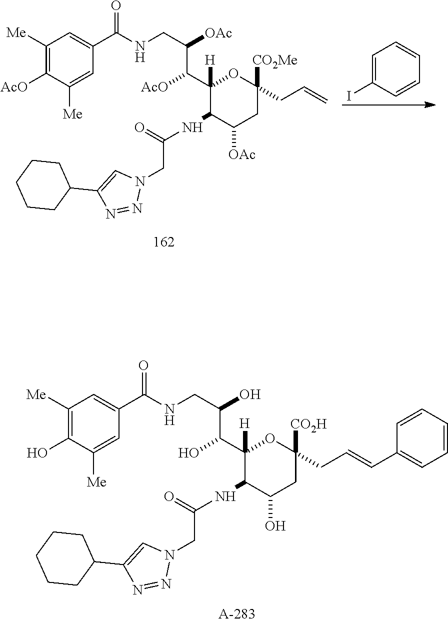

To a stirred solution of (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 26 mg, 0.032 mmol) in MeCN (0.75 mL) was added iodobenzene (13.07 mg, 0.064 mmol) and methyl dicyclohexylamine (20.6 μl, 0.096 mmol). The mixture was purged with N₂ for 30 min, after which time bis(tri-tert-butylphosphine)palladium (1.637 mg, 3.202 μmol) was added followed by warming to 80° C., and stirring for 3 h. The completed reaction mixture was purified directly onto a Biotage Ultra SNAP silica gel column (10 g) eluting with 5 CV gradient of 0 to 100% EtOAc in heptane followed by a 5 CV gradient of 0 to 10% MeOH in EtOAc. The desired fractions were concentrated, and azeotroped to dry with MeOH (2×10 mL ea).

The crude residue from above was dissolved with stirring in MeOH (0.6 mL) followed by the addition of 1 N aqueous sodium hydroxide (320 μl, 0.32 mmol), and stirred at room temperature for 26 h. The completed reaction mixture was purified directly by HPLC to provide A-283 (7.8 mg, 0.011 mmol, 34%) (MW_{Calc}+H=706.34; MW_{Obs}=706.5) after concentration of the desired fractions and drying under vacuum.

### Preparation of A-284 to A-287

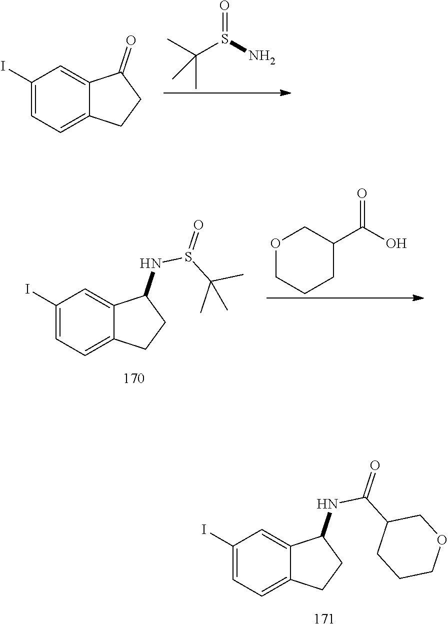

To a stirred solution of 2,3-dihydro-6-iodoinden-1-one (362 mg, 1.403 mmol) in THF (1.81 mL) at room temperature was added titanium isopropoxide (1.044 mL, 3.507 mmol) and R-(+) tert-butansulfinamide (230 mg, 1.894 mmol). The reaction mixture was warmed to 60° C. and stirred for 4 hours, after which time it was cooled to room temperature followed by the addition of THF (0.3 mL) and cooling to −78° C. 1 M L-Selectride in THF (1.894 mL, 1.894 mmol) was added dropwise over 5 min maintaining the temperature below −70° C. followed by stirring for an additional 30 min. The completed reaction was quenched with a mixture of water: conc. H₂SO₄:sat. Na₂SO₄ (20:1:1, (5 mL)) followed by warming to room temperature with stirring. The resultant mixture was extracted with a 1:1 mixture of EtOAc:toluene (3×5 mL ea), and the combined organic layers were washed sat NH₄Cl (5 mL), brine (5 mL), and dried over Na₂SO₄, filtered and concentrated to dry. The residue was purified over a Biotage Ultra SNAP silica gel column (4 g) eluting with 5 CV gradient of 0 to 100% EtOAc in heptane to provide compound 170 (149 mg, 0.410 mmol, 29%) (MW_{Calc}+Na=386.02; MW_{Obs}=386.02) after concentration of the desired fractions and drying under vacuum.

To a stirred solution of N—((S)-6-iodo-2,3-dihydro-1H-inden-1-yl)-2-methylpropane-2-sulfinamide (170, 280 mg, 0.771 mmol) in THF (3 mL) at room temperature was added conc HCl (268 μl, 3.083 mmol). The resultant solid suspension was stirred for 1 h, after which time it was slowly quenched with sat. NaHCO₃ (5 mL) followed by 1N NaOH (5 mL). The resultant mixture was extracted with EtOAc (2×10 mL ea), and the combined organic layers were washed with brine (5 mL), dried over Na₂SO₄, filtered and concentrated to dry. The crude oil was dissolved in MeCN (3 mL) at room temperature with stirring followed by the addition of tetrahydro-2H-pyran-3-carboxylic acid (125 mg, 0.964 mmol), triethylamine (430 μl, 3.083 mmol) and HATU (366 mg, 0.964 mmol). The reaction suspension was diluted with MeCN (3 mL) followed by stirring for an additional 3 h. The completed reaction was diluted with EtOAc (10 mL), washed with sat. NaHCO₃ (5 mL) and brine (5 mL), followed by dried over Na₂SO₄, filtered and concentrated to dry. The residue was purified over a Biotage Ultra SNAP silica gel column (10 g) eluting with 10 CV gradient of 0 to 100% EtOAc in heptane to provide compound 171 (265 mg, 0.714 mmol, 93%) (MW_{Calc}+Na=394.04; MW_{Obs}=394.03) after concentration of the desired fractions and drying under vacuum.

A-284 was prepared in a similar fashion to A-283 starting with compound 162 (25 mg, 0.031 mmol) and N—((S)-6-iodo-2,3-dihydro-1H-inden-1-yl)tetrahydro-2H-pyran-3-carboxamide (171, 34.3 mg, 0.092 mmol) in MeCN (1.5 mL) with methyl dicyclohexylamine (26 uL, 0.123 mmol) and bis(tri-tert-butylphosphine)palladium (1.6 mg, 0.003 mmol) to provide A-284 (7.0 mg, 0.008 mmol, 26%) (MW_{Calc}+H=873.44; MW_{Obs}=873.39) after deacetylation, ester hydrolysis, HPLC purification, and rendering to dryness under vacuum

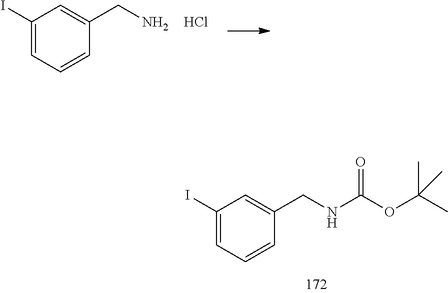 [SMILES: NCc1cccc(I)c1 ; Cl ; CC(C)(C)OC(=O)NCc1cccc(I)c1]

To a stirred solution of 3-iodobenzylamine hydrochloride (516 mg, 1.915 mmol) in THF (6.192 mL) at room temperature was added triethylamine (801 μl, 5.744 mmol) and BOC-anhydride (600 μl, 2.585 mmol). The reaction was stirred for 1 h, after which time the completed reaction was diluted with EtOAc (20 mL), washed with sat. NaHCO₃ (5 mL) and brine (5 mL), followed by dried over Na₂SO₄, filtered and concentrated to dry. The residue was purified over a Biotage Ultra SNAP silica gel column (25 g) eluting with 10 CV gradient of 0 to 50% EtOAc in heptane to provide compound 172 (554 mg, 1.663 mmol, 87% yield) as a crystalline solid after concentration of the desired fractions and drying under vacuum.

A-285 was prepared in a similar fashion to A-283 starting with compound 162 (280 mg, 0.345 mmol) and tert-butyl 3-iodobenzylcarbamate (172, 345 mg, 1.035 mmol) in MeCN (2.8 mL) with methyl dicyclohexylamine (332 uL, 1.552 mmol) and bis(tri-tert-butylphosphine)-palladium (17.6 mg, 0.034 mmol) to provide A-285 (4.5 mg, 0.005 mmol, 30%) (MW_{Calc}+H=835.42; MW_{Obs}=835.6) upon using (18 mg, 0.018 mmol) of the protected intermediate followed by after deacetylation, ester hydrolysis, HPLC purification, and rendering to dryness under vacuum.

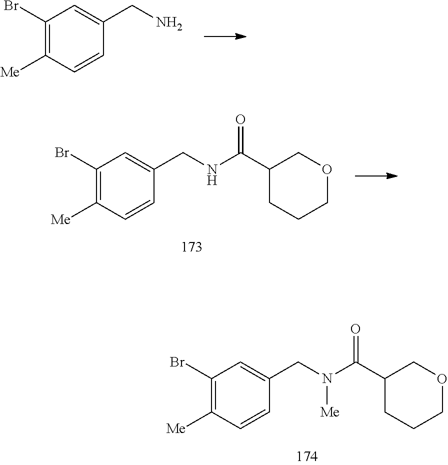 [SMILES: Cc1ccc(CN)cc1Br ; Cc1ccc(CNC(=O)C2CCCOC2)cc1Br ; Cc1ccc(CN(C)C(=O)C2CCCOC2)cc1Br]

To a stirred suspension of (3-bromo-4-methylphenyl)methanamine (214 mg, 1.069 mmol) in DCM (1.59 mL) at room temperature was added tetrahydro-2H-pyran-3-carboxylic acid (107 mg, 0.822 mmol) followed by HATU (500 mg, 1.315 mmol) and TEA (573 μl, 4.111 mmol). The reaction mixture was stirred for 16 h, after which time it was quenched with sat. NaHCO₃ (10 mL), and then extracted with MTBE (2×20 mL ea). The combined organic layers were dried over Na₂SO₄, filtered and concentrated. The resultant residue was purified over a Biotage Ultra SNAP silica gel column (10 g) eluting with 5 CV gradient of 0 to 40% EtOAc then a 10 CV gradient of 40 to 80% EtOAc in heptane to provide compound 173 (198 mg, 0.634 mmol, 77% yield) (MW_{Calc}+H=314.05; MW_{Obs}=314.02) after concentration of the desired fractions and drying under vacuum.

To a stirred solution of N-(3-bromo-4-methylbenzyl)tetrahydro-2H-pyran-3-carboxamide (173, 150 mg, 0.48 mmol) in THF (3.0 mL) at 0° C. was added and then sodium hydride (38.4 mg, 0.961 mmol). The mixture was stirred for 5 min, after which time iodomethane (0.060 mL, 0.961 mmol). The reaction mixture was warmed to room temperature and stirred for 2 h, after which time water (2 mL) was slowly added. The quenched mixture was extracted with EtOAc (3×2 mL ea), and the combined organic layers were dried over Na₂SO₄, filtered and concentrated. The resultant residue was purified over a Biotage Ultra SNAP silica gel column (10 g) eluting with 5 CV gradient of 0 to 65% EtOAc provide compound 174 (123 mg, 0.377 mmol, 78% yield) (MW_{Calc}+Na=348.07; MW_{Obs}=348.12) after concentration of the desired fractions and drying under vacuum.

A-286 was prepared in a similar fashion to A-283 starting with compound 162 (50 mg, 0.062 mmol) and N-(3-bromo-4-methylbenzyl)tetrahydro-2H-pyran-3-carboxamide (51 (171), 38.5 mg, 0.123 mmol) in MeCN (1.2 mL) with methyl dicyclohexylamine (40 uL, 0.185 mmol) and bis(tri-tert-butylphosphine)palladium (3.2 mg, 0.006 mmol) to provide A-286 (13.5 mg, 0.015 mmol, 25%) (MW_{Calc}+H=861.44; MW_{Obs}=861.6) after deacetylation, ester hydrolysis, HPLC purification, and rendering to dryness under vacuum.

A-287 was prepared in a similar fashion to A-283 starting with compound 162 (50 mg, 0.062 mmol) and N-(3-bromo-4-methylbenzyl)-N-methyltetrahydro-2H-pyran-3-carboxamide (52 (172), 50.2 mg, 0.154 mmol) in MeCN (1.2 mL) with methyl dicyclohexylamine (46 uL, 0.216 mmol) and bis(tri-tert-butylphosphine)palladium (4.7 mg, 0.009 mmol) to provide A-287 (10.1 mg, 0.011 mmol, 18.7%) (MW_{Calc}+H=875.42; MW_{Obs}=875.5) after deacetylation, ester hydrolysis, HPLC purification, and rendering to dryness under vacuum.

### Preparation of A-288

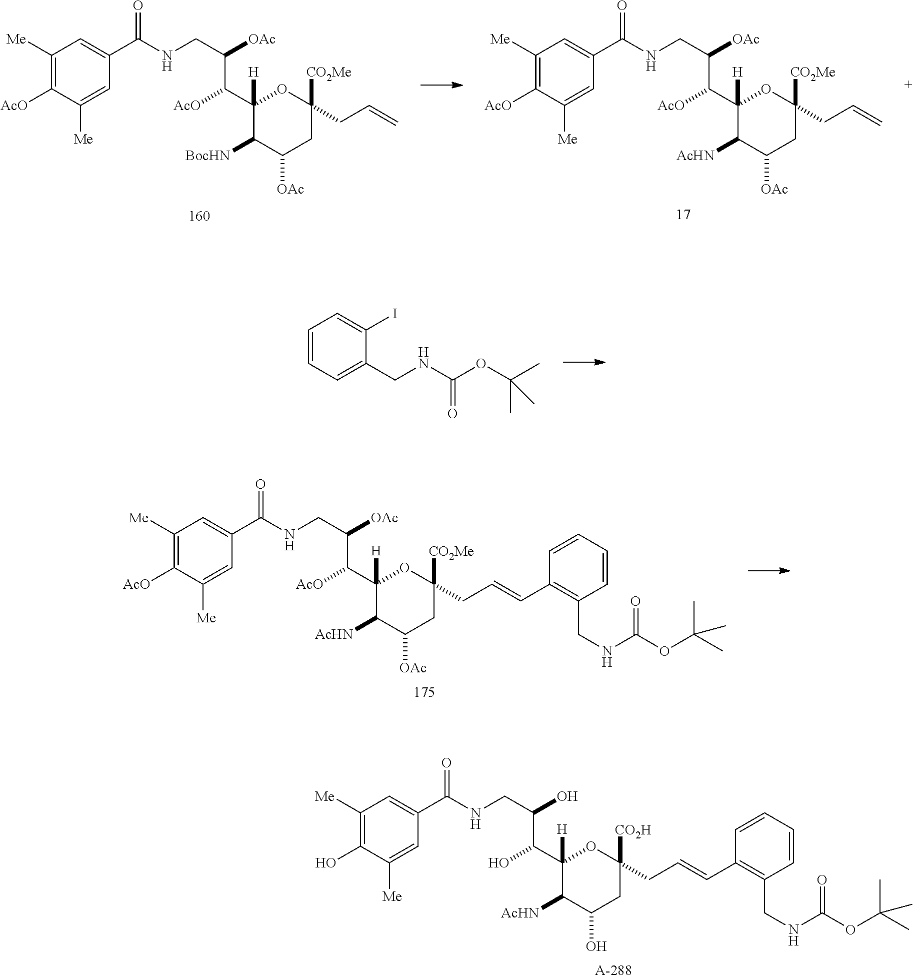

To a stirred solution of (1R,2R)-3-(4-acetoxy-3,5-dimethylbenzamido)-1-((2S,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (160, 427 mg, 0.592 mmol) in DCM (3.0 mL) at room temperature was added TFA (3.0 mL, 38.797 mmol). The reaction mixture was stirred for 10 min, after which time it was concentrated, and azeotroped to dryness with toluene (3×20 mL ea). The resultant residue was dissolved in THF (8.54 mL) with stirring followed by 10% aq sodium bicarbonate (1.49 mL, 1.777 mmol), and then acetic anhydride (0.112 mL, 1.185 mmol). The final reaction mixture was stirred at room temperature for 30 min, after which time brine (10 mL) was added followed by extracting the mixture with EtOAc (2×20 mL ea). The combined organic layers were washed with brine (5 mL), dried over Na2SO4, filtered and concentrated. The resultant residue was purified over a Biotage SNAP Ultra silica gel column (10 g) eluting with 10 CV of 0-100% EtOAc in heptane to provide compound 17 (340 mg, 0.513 mmol, 87% yield) (MW_{Calc}+H=663.27; MW_{Obs}=663.27) after collection of the desired fractions, concentration, and drying under high vacuum.

To a stirred solution of (1R,2R)-1-((2S,3R,4S,6R)-3-acetamido-4-acetoxy-6-allyl-6-(methoxycarbonyl)tetrahydro-2H-pyran-2-yl)-3-(4-acetoxy-3,5-dimethylbenzamido)propane-1,2-diyl diacetate (17, 58 mg, 0.088 mmol) in acetonitrile (1 mL) at room temperature under N₂ atmosphere was added tert-butyl 2-iodobenzylcarbamate (87 mg, 0.263 mmol), and then methyl dicyclohexylamine (67.5 μL, 0.315 mmol) followed by bis(tri-tert-butylphosphine)palladium (5.4 mg, 0.010.503 μmol). The reaction vessel was sealed followed by warming to 85° C. and then stirred for 8 h. The completed reaction was cooled to room temperature, and then applied directly unto a Biotage SNAP Ultra silica gel column (10 g) eluting with 10 CV of 0-100% EtOAc in heptane to provide compound 175 (74 mg, 0.085 mmol, 97% yield) (MW_{Calc}+Na=890.38; MW_{Obs}=890.26) after collection of the desired fractions, concentration, and drying under high vacuum.

To a stirred solution of (1R,2R)-1-((2S,3R,4S,6R)-3-acetamido-4-acetoxy-6-((E)-3-(2-(((tert-butoxycarbonyl)amino)methyl)phenyl)allyl)-6-(methoxycarbonyl)tetrahydro-2H-pyran-2-yl)-3-(4-acetoxy-3,5-dimethylbenzamido)propane-1,2-diyl diacetate (175, 18 mg, 0.021 mmol) in methanol at room temperature (0.839 μl, 0.021 mmol) was added 1 M aqueous solution of sodium hydroxide (415 μl, 0.415 mmol). The reaction mixture was stirred for 24 h, after which time the mixture was acidified with 1 N HCl (0.5 mL), diluted with methanol to 2 mL total and purified over an HPLC column to provide A-288 (14 mg, 0.020 mmol, 97%) (MW_{Calc}+H=686.32; MW_{Obs}=686.31; 1HNMR=Y) after collection of the desired fractions, concentration, and drying under high vacuum.

### Preparation of A-289 to A-291

A-289 was prepared in a similar fashion to A-288 starting with compound 17 (32 mg, 0.048 mmol) coupling with commercially available N-(3-bromo-4-methylbenzyl)tetrahydro-2H-pyran-3-carboxamide (18.1 mg, 0.058 mmol) in acetonitrile (0.53 mL) in the presence of N-cyclohexyl-N-methylcyclohexanamine (46 uL, 0.217 mmol) and bis(tri-tert-butylphosphine)-palladium(0) (2.5 mg, 0.005 mmol) to provide A-289 (15.1 mg, 0.021 mmol, 44%) (MW_{Calc}+H=712.34; MW_{Obs}=712.5) and A-290 (2 mg, 0.003 mmol, 6%) (MW_{Calc}+H=712.34; MW_{Obs}=712.5) after deacetylation, ester hydrolysis, HPLC purification, and rendering to dryness under vacuum the two isolated products.

A-291 was prepared in a similar fashion to A-288 starting with compound 17 (44 mg, 0.066 mmol) coupling with commercially available 3-(3-bromophenyl)isoxazol-5-amine (47.6 mg, 0.199 mmol) in acetonitrile (0.53 mL) in the presence of N-cyclohexyl-N-methylcyclohexanamine (57 uL, 0.266 mmol) and bis(tri-tert-butylphosphine)-palladium(0) (3.4 mg, 0.007 mmol) to provide A-291 (15.1 mg, 0.021 mmol, 44%) (MW_{Calc}+H=639.26; MW_{Obs}=639.4) after deacetylation, ester hydrolysis, HPLC purification, and rendering to dryness under vacuum the two isolated products.

### Preparation of A-292 to A-295

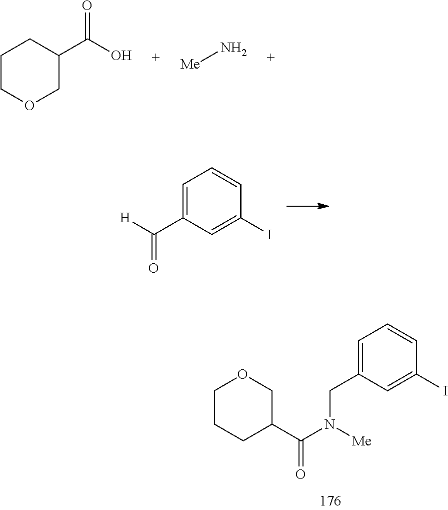 [SMILES: O=C(O)C1CCCOC1 ; CN ; O=Cc1cccc(I)c1 ; CN(Cc1cccc(I)c1)C(=O)C1CCCOC1]

To a stirred solution of commercially available 3-iodobenzaldehyde (260 mg, 1.121 mmol) in EtOH (3 mL) at room temperature was added a 33% solution of methylamine in methanol (316 mg, 3.362 mmol). The mixture was stirred for 30 minutes, after which time sodium borohydride (63.6 mg, 1.681 mmol) was added followed by stirring for an additional 1 h. The total mixture was diluted with DCM (30 mL), filtered over a plug of Celite (5 g), eluting with DCM (2×5 mL ea), and the filtrate was concentrated to dry. The resulting residue was diluted with acetonitrile (3 mL), stirred at room temperature followed by the addition of tetrahydro-2H-pyran-3-carboxylic acid (146 mg, 1.121 mmol), triethyl amine (390 μl, 2.802 mmol), and HATU (426 mg, 1.121 mmol). The final reaction mixture was stirred at room temperature for 2 h, after which time water (5 mL) was added followed by extraction with EtOAc (3×10 mL ea). The combined organic layers were washed with water (5 mL), brine (5 mL), dried over Na₂SO₄, filtered and concentrated to dry. The residue was purified over a Biotage SNAP Ultra silica gel column (10 g) eluting with 10 CV 0 to 50% EtOAc in heptane to provide compound 176 (278 mg, 0.774 mmol, 69% yield) (MW_{Calc}+Na=382.04; MW_{Obs}=382.03) as a clear oil after collection of the desired fractions, concentration, and drying under vacuum.

A-292 was prepared in a similar fashion to A-288 starting with (1R,2R)-3-(4-acetoxy-3,5-dimethylbenzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (17, 25 mg, 0.035 mmol) coupled to N-(3-iodobenzyl)-N-methyltetrahydro-2H-pyran-3-carboxamide (176, 37.4 mg, 0.104 mmol) followed by NaOH hydrolysis and purification to provide A-292 (2.8 mg, 0.004 mmol, 11%) (MW_{Calc}+H=712.34; MW_{Obs}=712.39).

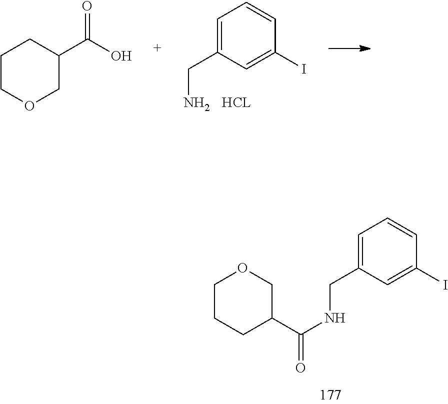

To a stirred suspension of (3-iodophenyl)methanaminium chloride (500 mg, 1.855 mmol) in DMF (7.9 mL) at room temperature was added tetrahydro-2H-pyran-3-carboxylic acid (314 mg, 2.412 mmol), (3-iodophenyl)methanaminium chloride (500 mg, 1.855 mmol), HATU (1.13 g, 2.97 mmol) and triethylamine (2.59 mL, 18.55 mmol). The reaction mixture was stirred for 16 h, after which time the completed reaction was diluted with sat. NaHCO₃ (10 mL), extracted with MTBE (2×20 mL ea). The combined organic layers were dried over Na2SO4, filtered and concentrated. The crude residue was purified over a Biotage SNAP Ultra silica gel column (25 g) eluting with 5 CV of 0 to 20% EtOAC in heptane to provide N-(3-iodobenzyl)tetrahydro-2H-pyran-3-carboxamide (177, 522 mg, 1.512 mmol, 82% yield) (MW_{Calc}+H=346.02; MW_{Obs}=346.06).

A-293 was prepared in a similar fashion to A-288 starting with (1R,2R)-3-(4-acetoxy-3,5-dimethylbenzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (17, 400 mg, 0.604 mmol) coupled to N-(3-iodobenzyl)tetrahydro-2H-pyran-3-carboxamide (177, 625 mg, 1.811 mmol) using methyl dicyclohexylamine (0.388 mL, 1.811 mmol), and bis(tri-tert-butylphosphine)-palladium (0) (31 mg, 0.06 mmol) in acetonitrile (7.2 mL) followed by NaOH hydrolysis and purification to provide A-293 (17.1 mg, 0.025 mmol, 50% starting with 0.050 mmol of the described compiled product) (MW_{Calc}+H=698.32; MW_{Obs}=698.5).

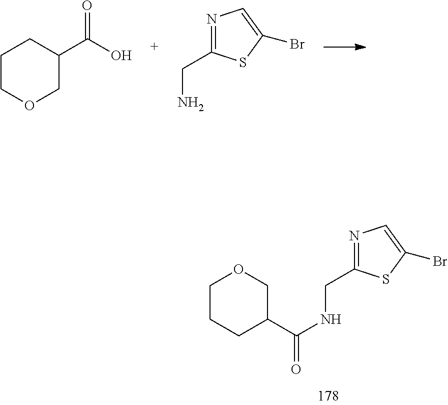 [SMILES: O=C(O)C1CCCOC1 ; NCc1ncc(Br)s1 ; O=C(NCc1ncc(Br)s1)C1CCCOC1]

To a stirred suspension of (5-bromothiazol-2-yl)methanamine (185 mg, 0.961 mmol) in acetonitrile (1.25 mL) at room temperature was added tetrahydro-2H-pyran-3-carboxylic acid (104.2 mg, 0.801 mmol) followed by HATU (487 mg, 1.281 mmol) and TEA (0.56 mL, 4.003 mmol). The reaction mixture was stirred after which time the reaction was diluted with sat. NaHCO₃ (10 mL), extracted with MTBE (2×20 mL ea.) The combined organic layers were dried over Na₂SO₄, filtered and concentrated. The crude product was purified over a silica gel column (10 g) eluting with 5 CV of 0 to 40% EtOAc in heptane, followed by 10 CV of 40 to 80% EtOAc in heptane to provide N-((5-bromothiazol-2-yl)methyl)tetrahydro-2H-pyran-3-carboxamide, 178 (202 mg, 0.662 mmol, 83% yield) (MW_{Calc}+H=306.99; MW_{Obs}=307.02).

A-294 was prepared in a similar fashion to A-288 starting with (1R,2R)-3-(4-acetoxy-3,5-dimethylbenzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (17, 45 mg, 0.068 mmol) coupled to N-((5-bromothiazol-2-yl)methyl)tetrahydro-2H-pyran-3-carboxamide (178, 25 mg, 0.081 mmol) using methyl dicyclohexylamine (0.065 mL, 0.306 mmol), and bis(tri-tert-butylphosphine)-palladium (0) (3.5 mg, 0.007 mmol) in dimethylacetamide (0.80 mL) followed by NaOH hydrolysis and purification to provide A-294 (1.6 mg, 0.002 mmol, 3% yield) (MW_{Calc}+H=705.28; MW_{Obs}=705.40).

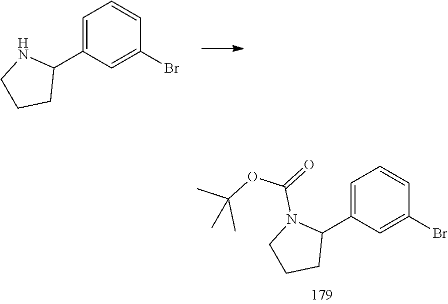 [SMILES: Brc1cccc(C2CCCN2)c1 ; CC(C)(C)OC(=O)N1CCCC1c1cccc(Br)c1]

To a stirred solution of 2-(3-Bromo-phenyl)-pyrrolidine (100 mg, 0.442 mmol) in THF (1.5 mL) at room temperature was added BOC-Anhydride (0.154 mL, 0.663 mmol) followed by stirring for 19 h. The crude completed mixture was purified directly over a Biotage SNAP Ultra silica gel column (10 g) eluting with 10 CV of 0 to 25% EtOAc in heptane to provide tert-butyl 2-(3-bromophenyl)-pyrrolidine-1-carboxylate, 179 (129 mg, 0.395 mmol, 89% yield) as a crystalline solid after collection of the desired fractions, concentration, and drying under vacuum.

A-295 was prepared in a similar fashion to A-288 starting with (1R,2R)-3-(4-acetoxy-3,5-dimethylbenzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (17, 75 mg, 0.035 mmol) coupled to tert-butyl 2-(3-bromophenyl)pyrrolidine-1-carboxylate (179, 111 mg, 0.34 mmol) followed by NaOH hydrolysis and purification to provide A-295 (66 mg, 0.091 mmol, 80%) (MW_{Calc}+H=726.36; MW_{Obs}=726.46).

### Preparation of A-296, A-297, and A-298

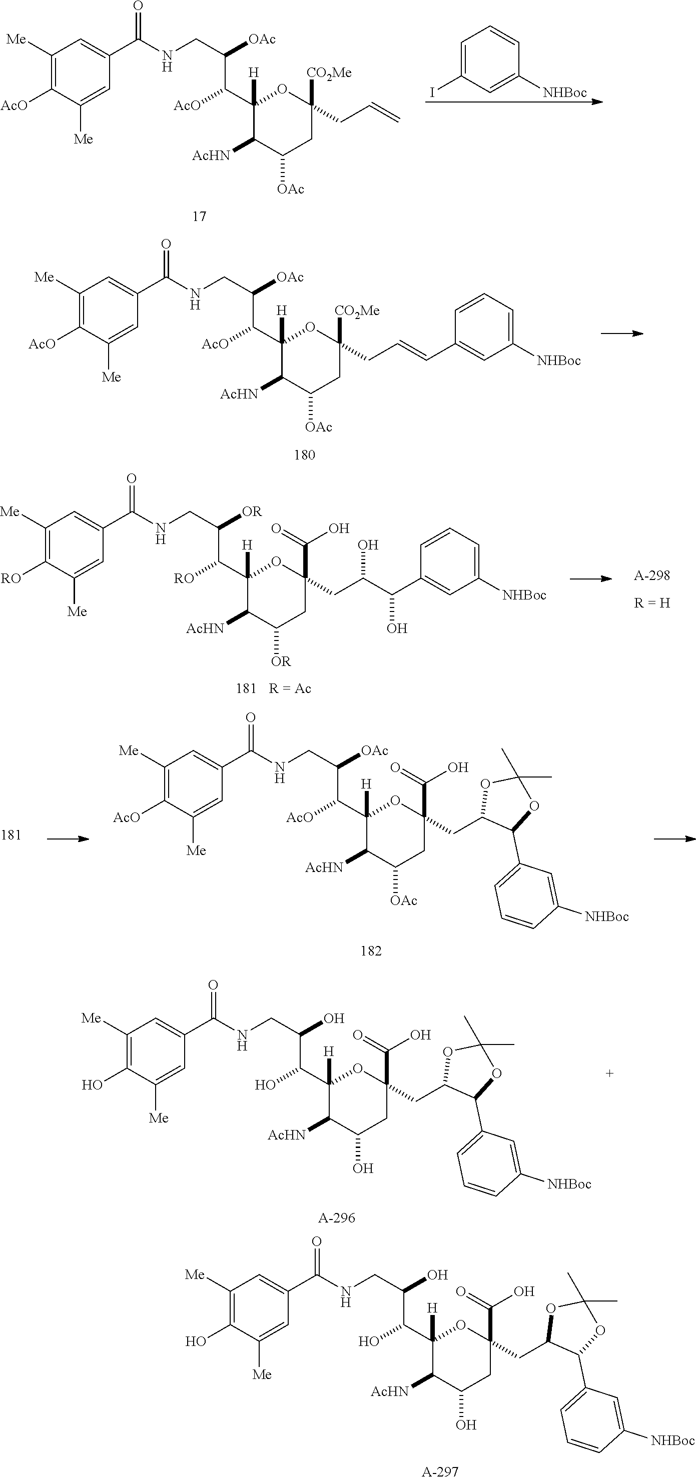

To a stirred solution of (1R,2R)-1-((2R,3R,4S,6R)-3-acetamido-4-acetoxy-6-allyl-6-(methoxycarbonyl)-tetrahydro-2H-pyran-2-yl)-3-(4-acetoxy-3,5-dimethylbenzamido)propane-1,2-diyl diacetate (17, 216 mg, 0.326 mmol) in acetonitrile (5.4 mL) was added tert-butyl (3-iodophenyl)carbamate (312 mg, 0.978 mmol) and N-cyclohexyl-N-methylcyclohexanamine (209 μl, 0.978 mmol) followed by purging with N₂ for 1 min, after which time bis(tri-tert-butylphosphine)palladium(0) (33.3 mg, 0.065 mmol) was added. The reaction vessel was sealed, warmed to 85° C., and stirred for 2 h. The completed reaction was filtered through a pad Celite (5 g) eluting with EtOAc (3×5 mL ea). The filtrate was concentrated and purified over a Biotage SNAP Ultra silica gel column (25 g) eluting with 3 CV of 50% EtOAc to heptane and a 10 CV gradient of to 50 to 100% EtOAc to heptane to provide impure compound 180 after concentration of the desired fractions. The impure material was re-purified over a Biotage SNAP Ultra silica gel column (25 g) eluting with 4 CV of 20% EtOAc in heptane and then 5 CV of 85% EtOAc to heptane to provide compound 180 (205 mg, 0.240 mmol, 74%) (MW_{Calc}+H=854.36; MW_{Obs}=854.56) after concentration of the desired fractions and drying under vacuum.

To a stirred solution of (1R,2R)-1-((2R,3R,4S,6R)-3-acetamido-4-acetoxy-6-((E)-3-(3-((tert-butoxycarbonyl)amino)phenyl)allyl)-6-(methoxycarbonyl)tetrahydro-2H-pyran-2-yl)-3-(4-acetoxy-3,5-dimethylbenzamido)propane-1,2-diyl diacetate (180, 70.0 mg, 0.082 mmol) in tert-butanol (0.7 mL) and water (0.7 mL) at 0° C. was added methanesulfonamide (13 mg, 0.137 mmol) followed by AD-mix-ALPHA (330 mg). The reaction mixture was allowed to warm to room temperature and stirred for 16 h. An additional amount of AD-mix-ALPHA (100 mg) was added and allowed to stir for 5 h. The completed reaction was cooled to 0° C. followed by the addition of 10% aqueous sodium bisulfite (1 mL) and stirred for an additional 15 min. The quenched reaction was extracted with DCM (4×5 mL ea), and the combined organic layers were washed with 1N HCl (2×5 mL ea) and water (5 mL). The organic layer was dried over sodium sulfate, filtered, concentrated and high vacuumed to dryness. Obtained compound 181 (52 mg, 0.060 mmol, 73%) (MW_{Calc}+Na=896.35; MW_{Obs}=896.17) as a solid. Used all in the next reaction.

To a stirred solution of crude compound 181 (52 mg, 0.060 mmol) in 1,2-dimethoxyethane (0.52 mL) and 2,2-dimethoxypropane (0.322 ml, 2.678 mmol) was added 1% sulfuric acid solution in 1,2-dimethoxyethane (0.1 mL) at room temperature. The reaction was stirred for 40 min after which time NaOH (0.012 ml, 0.012 mmol) was added and then concentrated. The residue was azeotroped to dryness with methanol (2×2 mL ea). The crude residue, compound 182, was used in the next step.

To a stirred solution of crude compound 182 (54 mg, 0.059 mmol) in methanol (0.520 ml) was added 1.0 N NaOH (1.0 ml, 1.00 mmol) at room temperature. The reaction was stirred for 16 h after which time the completed reaction was quenched with 1N HCl (1 mL) and submitted for purification to provide A-297 (12 mg, 0.017 mmol, 29%) (MW_{Calc}+H=746.35; MW_{Obs}=746.28), A-296(23 mg, 0.031 mmol, 52%) (MW_{Calc}+H=746.35; MW_{Obs}=746.28) and A-298 (1.6 mg, 0.002 mmol, 4%) (MW_{Calc}+H=746.35; MW_{Obs}=746.24) after collection of the appropriate fractions, concentration, and drying under vacuum.

### Preparation of A-299 and A-300

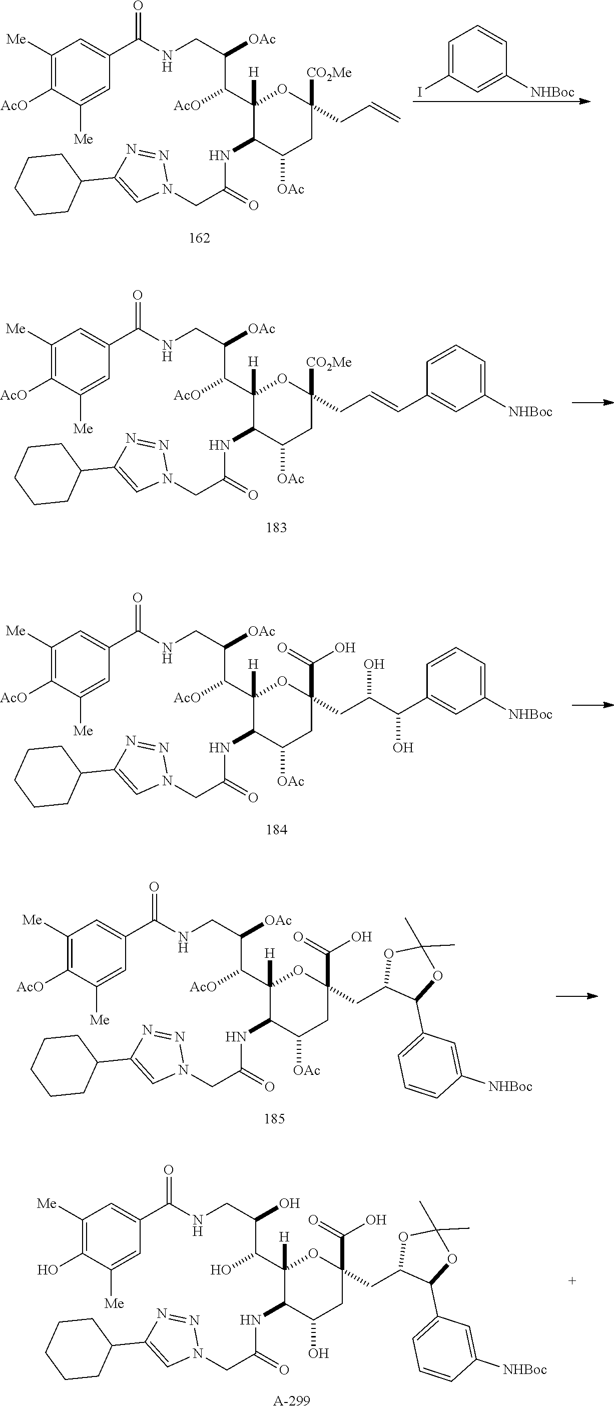

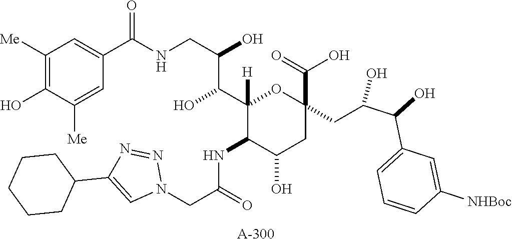

To a stirred solution of (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 65.0 mg, 0.08 mmol) in acetonitrile (0.65 mL) was added tert-butyl (3-iodophenyl)carbamate (38.3 mg, 0.12 mmol) and methyl dicyclohexylamine (34.3 μl, 0.16 mmol) followed by purging with N₂ for 10 min, after which time bis(tri-tert-butylphosphine)palladium (0) (4.09 mg, 8.006 μmol) was added. The reaction mixture was warmed to 80° C. and stirred for 24 h. The completed reaction was purified directly over a Biotage SNAP Ultra silica gel column (10 g) eluting with 2 CV of 20% EtOAc in heptane, 10 CV of a gradient of 20-100% EtOAc to heptane, 5 CV EtOAc, 5 CV of gradient of 0 to 20% MeOH in EtOAC, then 5 CV 20% MeOH in EtOAC to provide compound 183 (65 mg, 0.0.080 mmol, 81%) (MW_{Calc}+H=1003.46; MW_{Obs}=1003.47) after concentration of the desired fractions, and drying under vacuum.

To a stirred solution of (1R,2R)-3-(4-acetoxy-3,5-dimethylbenzamido)-1-((2R,3R,4S,6R)-4-acetoxy-6-((E)-3-(3-((tert-butoxycarbonyl)amino)phenyl)allyl)-3-(2-(4-cyclohexyl-1H-1,2,3-triazol-1-yl)acetamido)-6-(methoxycarbonyl)tetrahydro-2H-pyran-2-yl)propane-1,2-diyl diacetate (183, 26 mg, 0.026 mmol) in tert-butanol (0.130 ml, 1.359 mmol) and water (0.130 ml, 7.216 mmol) was cooled to 0° C. followed by AD-MIX-ALPHA (100 mg, 0.00 mmol). The reaction mixture was allowed to warm to room temperature and stirred for 24 h. Additional AD-MIX-ALPHA (100 mg, 0.00 mmol) was added, and the reaction was allowed to stir for an additional 48 h. The completed reaction was warmed to 0° C., and quenched with 10% aq NaHSO₃ (1 mL) and stirred for an additional 10 min. The resultant solution was extracted with DCM (4×5 mL ea) and concentrated to provide crude compound 184 (13 mg, 0.013 mmol, 49%) (MW_{Calc}-H=1021.45; MW_{Obs}=1021.38). The compound was used in the next reaction without further purification.

To a stirred solution of (2R,4S,5R,6R)-4-acetoxy-2-((2S,3S)-3-(3-((tert-butoxycarbonyl)amino)phenyl)-2,3-dihydroxypropyl)-5-(2-(4-cyclohexyl-1H-1,2,3-triazol-1-yl)acetamido)-6-((1R,2R)-1,2-diacetoxy-3-(4-acetoxy-3,5-dimethylbenzamido)propyl)tetrahydro-2H-pyran-2-carboxylic acid (184, 27 mg, 0.026 mmol) in dimethoxyethane (270 μL) was added 2,2-dimethoxypropane (10 μL, 0.083 mmol) followed by a solution of H₂SO₄ in DME (25 uL; from a stock solution of conc. H₂SO₄ (25 uL) in DME (0.6 mL)). The reaction was stirred for 1 h followed by additional 2,2-dimethoxypropane (10 μL, 0.083 mmol) DMP (10 μL, 0.083 mmol) and a solution of H₂SO₄ in DME (25 uL). The completed reaction was quenched with sat. aq. NaHCO₃ (3 mL) and extracted with DCM (3×3 mL ea). The combined organic layers were dried over MgSO4, filtered and concentrated to dry to provide crude compound 185 (13 mg, 0.012 mmol, 46%) (MW_{Calc}+H=1063.48; MW_{Obs}=1063.47), which was used in the next reaction without further purification.

To a stirred solution of (2S,4S,5R,6R)-4-acetoxy-2-(((4S,5S)-5-(3-((tert-butoxycarbonyl)amino)-phenyl)-2,2-dimethyl-1,3-dioxolan-4-yl)methyl)-5-(2-(4-cyclohexyl-1H-1,2,3-triazol-1-yl)acetamido)-6-((1R,2R)-1,2-diacetoxy-3-(4-acetoxy-3,5-dimethylbenzamido)propyl)tetrahydro-2H-pyran-2-carboxylic acid (185, 13.00 mg, 0.012 mmol) in methanol (0.26 mL) at room temperature was added 1 M aq. sodium hydroxide (245 μl, 0.245 mmol) followed by stirring for 24 h. The completed reaction was quenched with 1 N HCl (0.3 mL) and stirred for 10 min. The solution was purified directly by HPLC to provide A-299 (1.4 mg, 0.002 mmol, 13%) (MW_{Calc}+H=917.44; MW_{Obs}=917.35) and A-300 (1.0 mg, 0.001 mmol, 10%) (MW_{Calc}+H=855.41; MW_{Obs}=855.41) after concentration of the appropriate fractions and drying under vacuum.

### Preparation of A-301 and A-320

A-301 was prepared in a similar manner to A-299 starting with (1R,2R)-3-(4-acetoxy-3,5-dimethylbenzamido)-1-((2R,3R,4S,6R)-4-acetoxy-6-((E)-3-(3-(((tert-butoxycarbonyl)amino)methyl)phenyl)allyl)-3-(2-(4-cyclohexyl-1H-1,2,3-triazol-1-yl)acetamido)-6-(methoxycarbonyl)tetrahydro-2H-pyran-2-yl)propane-1,2-diyl diacetate (166, 49 mg, 0.048 mmol) to provide A-301 (3.0 mg, 0.003 mmol, 7% overall yield) (MW_{Calc}+Na=931.45; MW_{Obs}=931.32).

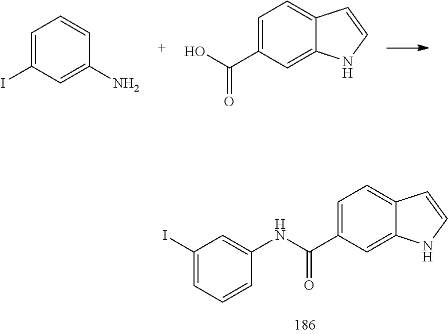 [SMILES: Nc1cccc(I)c1 ; O=C(O)c1ccc2cc[nH]c2c1 ; O=C(Nc1cccc(I)c1)c1ccc2cc[nH]c2c1]

To a stirred solution of 1H-indole-6-carboxylic acid (1.000 g, 6.205 mmol) in DMF (10.00 mL) at room temperature was added 3-iodoaniline (2.039 g, 9.308 mmol), TEA (1.730 ml, 12.41 mmol) followed by HATU (4.72 g, 12.41 mmol). The reaction mixture was stirred for 16 h, after which time water (20 mL) was added followed by extraction with EtOAc (3×10 mL ea). The combined organic layers were washed with sat. NaHCO₃ (10 mL), 1N HCl (10 mL), and then water (5 mL). The organic layer was concentrated and azeotroped to dry with acetonitrile (3×10 mL ea), then heptane (10 mL). The residue was purified directly over a Biotage SNAP Ultra silica gel column (25 g) eluting with 10 CV gradient of 20 to 100% EtOAc in heptane to provide compound 186 (755 mg, 2.085 mmol, 34%) (MW_{Calc}-H=360.99; MW_{Obs}=360.97) after concentration of the desired fractions and drying under vacuum.

A-302 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 27.0 mg, 0.041 mmol) and N-(3-iodophenyl)-1H-indole-6-carboxamide (186, 17.7 mg, 0.049 mmol) to provide A-302 (6.0 mg, 0.008 mmol, 19% overall yield) (MW_{Calc}+H=789.33; MW_{Obs}=789.24).

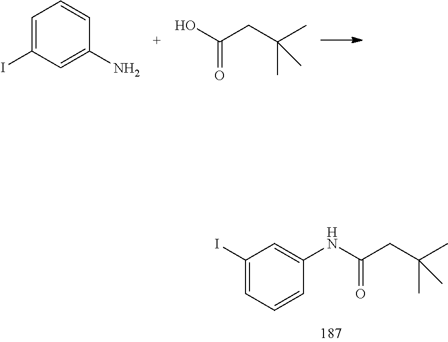

To a stirred solution of 3,3-dimethylbutanoic acid (0.318 g, 2.739 mmol) in DMF (3.00 mL) at room temperature was added 3-iodoaniline (0.30 g, 1.37 mmol), TEA (0.573 ml, 4.109 mmol) followed by HATU (1.042 g, 2.739 mmol). The reaction mixture was stirred for 16 h, after which time water (20 mL) was added followed by extraction with EtOAc (3×10 mL ea). The combined organic layers were washed with sat. NaHCO₃ (10 mL), 1N HCl (10 mL), and then water (5 mL). The organic layer was concentrated and azeotroped to dry with acetonitrile (3×10 mL ea), then heptane (10 mL). The residue was purified directly over a Biotage SNAP Dalton silica gel column (25 g) eluting with 10 CV gradient of 5 to 80% EtOAc in heptane to provide compound 187 (755 mg, 2.085 mmol, 34%) (MW_{Calc}-H=316.03; MW_{Obs}=316.56) after concentration of the desired fractions and drying under vacuum.

A-303 and A-304 were prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 18.0 mg, 0.027 mmol) and N-(3-iodophenyl)-3,3-dimethylbutanamide (187, 9.5 mg, 0.030 mmol) to provide A-303 (7.7 mg, 0.010 mmol, 38% overall yield) (MW_{Calc}+Na=766.36; MW_{Obs}=766.39), and A-304 (1.8 mg, 0.003 mmol, 9% overall yield) (MW_{Calc}+Na=726.36; MW_{Obs}=726.40).

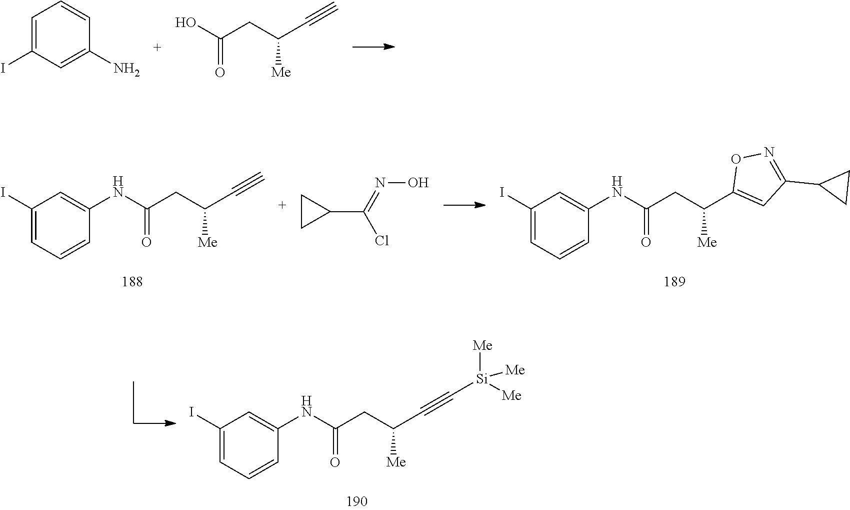

To a stirred solution of (R)-3-methylpent-4-ynoic acid (0.590 g, 5.262 mmol) and 3-iodoaniline (1.050 g, 4.794 mmol) in DMF (10.50 mL) at room temperature was added TEA (2.005 ml, 14.382 mmol) followed by HATU (3.65 g, 9.588 mmol). The reaction mixture was stirred for 16 h after which time the completed reaction was diluted with EtOAc (20 mL), heptane (20 mL), and water (10 mL). After stirring for an additional 5 min the layers were separated, and the aqueous layer was extracted with a 1:1 ratio of EtOAc:heptane (2×10 mL ea). The combined organic layers were washed with sat. NaHCO₃ (10 mL), 1N HCl (10 mL), and then water (5 mL). The organic layer was concentrated and azeotroped to dry with acetonitrile (3×10 mL ea), then heptane (10 mL). The residue was purified over a Biotage SNAP Dalton silica gel column (25 g) eluting with 10 CV gradient of 5 to 80% EtOAc in heptane to provide compound 188 (982 mg, 3.14 mmol, 65%) as a pale yellow solid after concentration of the desired fractions and drying under vacuum.

To a stirred solution of (R)—N-(3-iodophenyl)-3-methylpent-4-ynamide (188, 164 mg, 0.524 mmol) in THF (1.64 mL) at room temperature was added TEA (175 μl, 1.257 mmol) followed by a solution of the (Z)—N-hydroxycyclopropanecarbimidoyl chloride (140 mg, 1.171 mmol) in 0.5 mL THF. The suspension was stirred for 8 h, after which time the reaction was quenched with sat. NaHCO₃ (10 mL), and then extracted with EtOAc (2×10 mL ea). The combined organic layers were concentrated and azeotroped to dry with acetonitrile (3×10 mL ea), then heptane (10 mL). The purified over a Biotage SNAP Dalton silica gel column (25 g) eluting with 10 CV gradient of 20 to 50% EtOAc in heptane to provide compound 189 (98 mg, 0.247 mmol, 47%) (MW_{Calc}-H=395.03; MW_{Obs}=394.98) after concentration of the desired fractions and drying under vacuum.

A-305 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 25 mg, 0.038 mmol), and (R)-3-(3-cyclopropylisoxazol-5-yl)-N-(3-iodophenyl)butanamide (189, 19.43 mg, 0.049 mmol) to provide A-305 (1.0 mg, 0.001 mmol, 3% overall yield) (MW_{Calc}+Na=845.37; MW_{Obs}=845.39).

To a stirred solution of (R)—N-(3-iodophenyl)-3-methylpent-4-ynamide (188), 0.203 g, 0.648 mmol) and chlorotrimethylsilane (0.247 mL, 1.945 mmol) in THF (4.06 mL) under a N₂ atmosphere at −50° C. was added 1 M lithium bis(trimethylsilyl)amide (1.361 mL, 1.361 mmol) in THF dropwise over a 2 min period. The mixture was stirred for 3 h maintaining the temperature between −40 and −50° C., after which time additional chlorotrimethylsilane (0.10 mL, 0.787 mmol) was added followed by additional 1 M lithium bis(trimethylsilyl)amide (0.5 mL, 0.50 mmol) in THF. The resultant mixture was stirred at −40° C. for an additional 2 h, after which time it was quenched with sat. NH₄Cl, warmed to room temperature, and extracted with EtOAc (3×5 mL ea). The combined organic layers were washed with water (5 mL), dried over Na2SO4, filtered and concentrated to dry. The residue was purified over a Biotage SNAP Dalton silica gel column (25 g) eluting with 10 CV gradient of 5 to 20% EtOAc in heptane to provide compound 190 (181 mg, 0.470 mmol, 73%) (MW_{Calc}+H=386.04; MW_{Obs}=386.05) after concentration of the desired fractions and drying under vacuum.

A-306 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 57 mg, 0.086 mmol), and (R)—N-(3-iodophenyl)-3-methyl-5-(trimethylsilyl)pent-4-ynamide (190 36.5 mg, 0.095 mmol) to provide A-306 (0.7 mg, 0.001 mmol, 1% overall yield) (MW_{Calc}+H=740.33; MW_{Obs}=740.41) after desilylation of the penultimate intermediate with 1 N HCl (0.245 mL, 0.245 mmol) prior to final purification.

A-307 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 57 mg, 0.086 mmol) and directly using the osmium tetroxide conditions to provide A-307 (0.3 mg, 0.0005 mmol, 06% overall yield) (MW_{Calc}+H=555.25; MW_{Obs}=555.39).

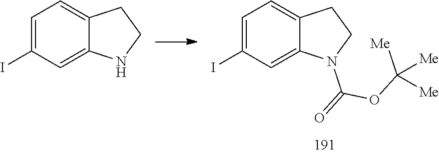 [SMILES: Ic1ccc2c(c1)NCC2 ; CC(C)(C)OC(=O)N1CCc2ccc(I)cc21]

To a stirred solution of 6-iodoindoline (0.1 g, 0.408 mmol) in THF (1.2 mL) at room temperature was added Et₃N (0.077 mL, 0.551 mmol) and DMAP (9.97 mg, 0.082 mmol) followed by BOC-anhydride (0.128 mL, 0.551 mmol). The reaction mixture was stirred for 24 h, after which time the completed reaction was diluted with EtOAc (10 mL), filtered over Celite (5 g), eluting with EtOAc (2×5 mL ea) followed by concentration to dry. The residue was purified over a Biotage SNAP Ultra silica gel column (10 g) eluting with 10 CV gradient of 0 to 25% EtOAc in heptane to provide compound 191 (54 mg, 0.156 mmol, 38% yield) after concentration of the desired fractions and drying under vacuum.

A-308 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 32 mg, 0.048 mmol), and tert-butyl 6-iodoindoline-1-carboxylate (191, 50 mg, 0.145 mmol) to provide A-308 (8.5 mg, 0.011 mmol, 34% overall yield) (MW_{Calc}+H=772.36; MW_{Obs}=772.29).

A-309 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 33 mg, 0.050 mmol), and 1-(3-bromophenyl)pyrrolidin-2-one (35.9 mg, 0.149 mmol) to provide A-309 (17.1 mg, 0.025 mmol, 50% overall yield) (MW_{Calc}+H=714.32; MW_{Obs}=714.30).

A-310 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 42 mg, 0.063 mmol), and 5-(3-bromophenyl)-1,3-oxazole (42.6 mg, 0.190 mmol) to provide A-310 (4.0 mg, 0.005 mmol, 8% overall yield) (MW_{Calc}+H=760.30; MW_{Obs}=760.30)

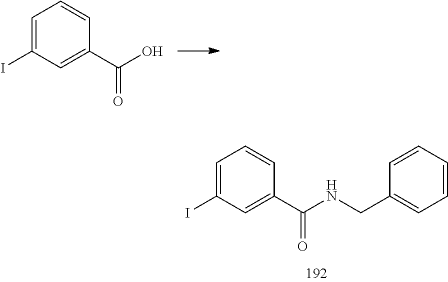 [SMILES: O=C(O)c1cccc(I)c1 ; O=C(NCc1ccccc1)c1cccc(I)c1]

To a stirred solution of 3-iodobenzoic acid (1.0 g, 4.032 mmol) and phenylmethanamine (0.432 g, 4.032 mmol) in acetonitrile (10.00 mL) was added triethylamine (10.00 mL, 71.746 mmol) followed by HATU (2.300 g, 6.048 mmol) at room temperature. The reaction was stirred for 3 h, after which time it was quenched with water (20 mL) and stirred for 2 h. The resulting precipitate was filtered off, and rinsed with water (3×5 mL ea). The resultant filter cake was dried at room temperature under vacuum for 24 h to provide compound 192 (1.1 g, 3.26 mmol, 81%) as a white solid.

A-311 and A-312 were prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 40 mg, 0.060 mmol), and N-benzyl-3-iodobenzamide (190, 61.1 mg, 0.181 mmol) to provide A-311 (5.0 mg, 0.007 mmol, 11% overall yield) (MW_{Calc}+Na=786.33; MW_{Obs}=786.34) and A-312 (2.6 mg, 0.003 mmol, 6% overall yield (MW_{Calc}+Na=786.33; MW_{Obs}=786.30).

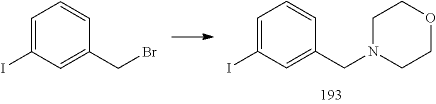 [SMILES: BrCc1cccc(I)c1 ; Ic1cccc(CN2CCOCC2)c1]

To a stirred solution of 1-(bromomethyl)-3-iodobenzene (341 mg, 1.148 mmol) in MeCN (1.00 mL) at room temperature was added morpholine (100 mg, 1.148 mmol) and potassium carbonate (159 mg, 1.148 mmol). The reaction mixture was stirred for 16 h, after which time it was filtered over Celite (2 g) eluting with DCM (3×2 mL ea), and concentrated to dryness. The residue was purified over a Biotage SNAP Ultra silica gel column (10 g) eluting with 10 CV gradient of 0 to 100% EtOAc in heptane to provide compound 193 (211 mg, 0.696 mmol, 60.6% yield) (MW_{Calc}+H=304.01; MW_{Obs}=304.04) after concentration of the desired fractions and drying under vacuum.

A-313 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 44 mg, 0.066 mmol), and 4-(3-iodobenzyl)morpholine (193, 40.3 mg, 0.133 mmol) to provide A-313(4.3 mg, 0.006 mmol, 9% overall yield) (MW_{Calc}+H=730.35; MW_{Obs}=730.46).

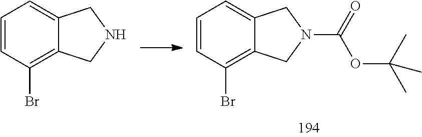 [SMILES: Brc1cccc2c1CNC2 ; CC(C)(C)OC(=O)N1Cc2cccc(Br)c2C1]

Compound 194 was prepared in a similar fashion to compound 191 starting with 4-bromoiso-indoline hydrochloride (0.25 g, 1.066 mmol) and BOC-anhydride (0.309 ml, 1.333 mmol) to provide compound 194 (310 mg, 1.040 mmol, 98%).

A-314 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 42 mg, 0.063 mmol), and tert-butyl 4-bromoisoindoline-2-carboxylate (44.5 mg, 0.149 mmol) to provide A-314 (7.9 mg, 0.010 mmol, 16% overall yield) (MW_{Calc}+H=772.36; MW_{Obs}=772.47).

A-315 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 33 mg, 0.050 mmol), and tert-butyl 2-(3-bromophenyl)acetate (43.0 mg, 0.158 mmol) to provide A-315 (21 mg, 0.028 mmol, 44% overall yield) (MW_{Calc}+H=745.35; MW_{Obs}=745.38).

A-316 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 35 mg, 0.053 mmol), and 1-(3-bromophenyl)piperidin-2-one (33.6 mg, 0.132 mmol) to provide A-316 (13.0 mg, 0.018 mmol, 34% overall yield) (MW_{Calc}+H=728.33; MW_{Obs}=728.45).

A-317 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 30 mg, 0.045 mmol), and 1-(5-bromo-2-fluorophenyl)pyrrolidin-2-one (29.2 mg, 0.113 mmol) to provide A-317 (8.0 mg, 0.011 mmol, 24% overall yield) (MW_{Calc}+H=732.31; MW_{Obs}=732.37).

A-318 was prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 30 mg, 0.045 mmol), and 1-(3-bromo-4-methylphenyl)pyrrolidin-2-one (28.8 mg, 0.113 mmol) to provide A-318 (6 mg, 0.008 mmol, 18% overall yield) (MW_{Calc}+H=728.33; MW_{Obs}=728.38).

A-319 and A-320 were prepared in a similar manner to A-299 starting with (1R,2R)-3-(4-acetoxy-3,5-dimethylbenzamido)-1-((2R,3R,4S,6R)-4-acetoxy-6-allyl-3-(2-(4-cyclohexyl-1H-1,2,3-triazol-1-yl)acetamido)-6-(methoxycarbonyl)tetrahydro-2H-pyran-2-yl)propane-1,2-diyl diacetate (162, 30 mg, 0.045 mmol), and 1-(5-bromo-2-methylphenyl)pyrrolidin-2-one (28.8 mg, 0.113 mmol) to provide A-319 (2.0 mg, 0.003 mmol, 6% overall yield) (MW_{Calc}+H=728.33; MW_{Obs}=728.28) and A-320 (8.6 mg, 0.01212 mmol, 26% overall yield) (MW_{Calc}+Na=750.33; MW_{Obs}=750.31).

### Preparation of A-321 and A-321

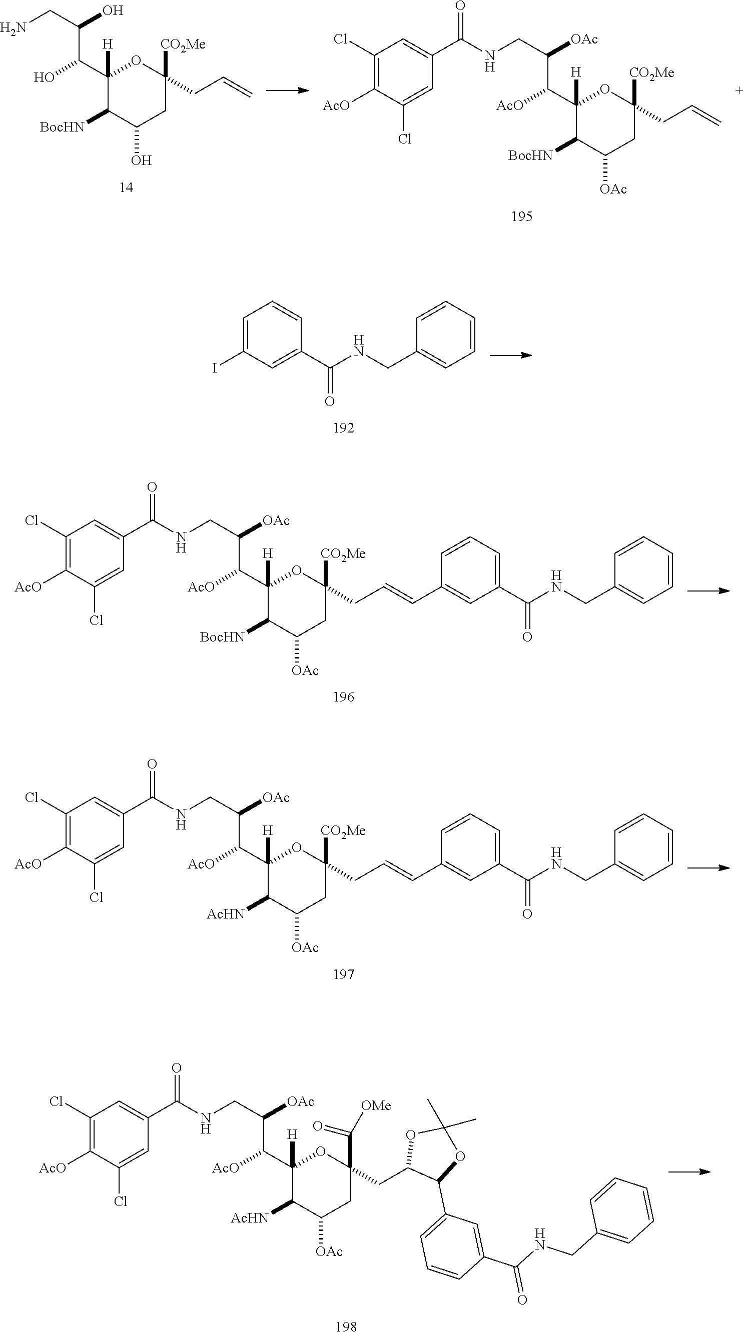

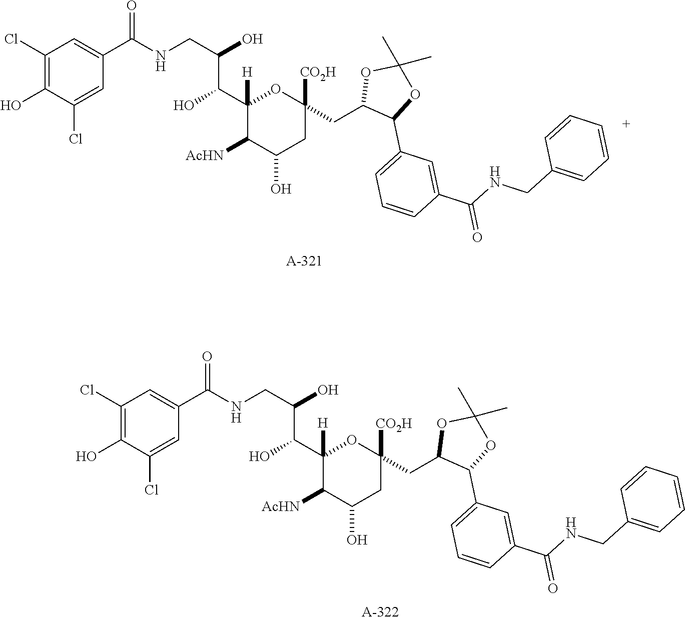

To a stirred solution of (2R,4S′,5R,6R)-methyl 2-allyl-6-(1R,2R)-3-amino-1,2-dihydroxypropyl)-5-((tert-butoxycarbonyl)amino)-4-hydroxytetrahydro-2H-pyran-2-carboxylate (14, 470 mg, 1.162 mmol) in acetonitrile (10 mL) at room temperature was added 3,5-dichloro-4-hydroxybenzoic acid (361 mg, 1.743 mmol) and triethylamine (0.486 mL, 3.486 mmol). The mixture was stirred until homogeneous followed by the addition of 1H-benzo[d][1,2,3]triazol-1-ol (79 mg, 0.581 mmol), and then N1-((ethylimino)methylene)-N3,N3-dimethylpropane-1,3-diamine hydrochloride (446 mg, 2.324 mmol). The final mixture was stirred for 16 h, after which time it was quenched with water (10 mL) and diluted with ethyl acetate (20 mL) with mixing. The layers were separated, and the aqueous layer was extracted with ethyl acetate (3×5 mL ea). The combined organic layers were washed with brine (10 mL), dried over Na2SO4, filtered, and concentrated under reduced pressure on the rotovap to afford a yellow/white solid. The resultant solid was dissolved into pyridine (10 ml, 123.641 mmol) at room temperature followed by the dropwise addition of acetic anhydride (3.0 ml, 31.737 mmol). The final reaction mixture was stirred for 16 h, after which time the reaction mixture was concentrated, and purified over a Biotage SNAP Ultra silica gel column (25 g) eluting with 2 CV of heptane then a 8 CV gradient of 0 to 100% EtOAc in heptane, followed by 2 CV of EtOAc to provide compound 195 (365 mg, 0.479 mmol, 41% yield) (MW_{Calc}+Na=783.20; MW_{Obs}=783.16) after concentration of the desired fractions and drying under vacuum.

To a stirred solution of (1R,2R)-3-(4-acetoxy-3,5-dichlorobenzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (195, 30 mg, 0.039 mmol) and N-benzyl-3-iodobenzamide (192, 15 mg, 0.043 mmol) in acetonitrile (0.60 mL) at room temperature was added methyl dicyclohexylamine (0.034 ml, 0.158 mmol) followed by bubbling with N₂ for 10 min, after which time bis(tri-tert-butylphosphine)-palladium (0) (4.03 mg, 7.878 μmol) was added. The reaction vessel was sealed, heated to 80° C., and stirred for 16 h. The completed reaction was cooled to room temperature, and then purified directly onto a Biotage SNAP Ultra silica gel column (10 g) eluting with 5 CV gradient of 0 to 100% EtOAc in heptane, 5 CV of EtOAc, then a 5 CV gradient of 0 to 10% MeOH in EtOAc provide compound 196 (36 mg, 0.039 mmol, 98% yield) (MW_{Calc}+H=970.29; MW_{Obs}=970.46) after concentration of the desired fractions and drying under vacuum.

To a stirred solution (1R,2R)-3-(4-acetoxy-3,5-dichlorobenzamido)-1-((2R,3R,4S,6R)-4-acetoxy-6-((E)-3-(3-(benzylcarbamoyl)phenyl)allyl)-3-((tert-butoxycarbonyl)amino)-6-(methoxycarbonyl)-tetrahydro-2H-pyran-2-yl)propane-1,2-diyl diacetate (196, 10.00 mg, 0.010 mmol) in DCM (0.2 mL) at room temperature was added TFA (0.1 mL, 1.298 mmol). The reaction mixture was stirred for 25 min, after which time it was concentrated and azeotroped to dry with toluene (2×10 mL). The resultant residue was dissolved in pyridine (0.100 mL) at room temperature followed by the addition of acetic anhydride (8 μL, 0.085 mmol). The final mixture was stirred for 30 min, after which time it was quenched with water (0.5 mL) and stirred for 1 h. The mixture was extracted with EtOAc (3×5 mL ea), and the combined organic layers were washed with 0.1 N HCl (10 mL), and water (5 mL). The organic layer was dried over Na2SO4, filtered and concentrated to dryness to provide crude compound 197 (9 mg, 0.010 mmol, 96%) (MW_{Calc}+H=912.24; MW_{Obs}=912.41) without further purification.

To a stirred solution (1R,2R)-1-((2R,3R,4S,6R)-3-acetamido-4-acetoxy-6-((E)-3-(3-(benzylcarbamoyl)-phenyl)allyl)-6-(methoxycarbonyl)tetrahydro-2H-pyran-2-yl)-3-(3,5-dichloro-4-hydroxybenzamido)-propane-1,2-diyl diacetate (197, 9.00 mg, 10.336 μmol) in tBuOH (0.180 mL) and water (0.180 mL) was added methanesulfonamide (3.93 mg, 0.041 mmol) and the solution was cooled to 0° C. A solution of 1 M AD-MIX-ALPHA (10 uL, 10.0 μmol) was added, and the reaction mixture was warmed to room temperature and stirred for 16 h. The completed reaction was quenched with sat. aq. NaHSO₃ (2 mL) followed by DCM (2 mL) and stirred for an additional 15 min. The layers were separated, and the aqueous layer was extracted with DCM (3×2 mL ea). The combined organic layers were washed with 1 N HCl (2 mL), and the resulting aqueous layer was extracted with DCM (1 mL). 2,2-Dimethylpropane (3 mL) was added to the combined organic layers, the solution was concentrated to 5 mL, and stirred for 1 h. The mixture was diluted with DCM (5 mL) that was previously washed with 1 N HCl (2 mL) followed by additional 2,2-dimethylpropane (3 mL), concentration to 5 mL, and stirred for 2 h. The completed reaction was quenched with sat. aq. NaHCO₃ (3 mL) and extracted with DCM (3×3 mL ea). The combined organic layers were dried over MgSO4, filtered and concentrated to dry to provide crude compound 198 (9 mg, 0.010 mmol, 94%) (MW_{Calc}+H=930.25; MW_{Obs}=930.51), which was used in the next reaction without further purification.

To a stirred solution (2S,4S,5R,6R)-5-acetamido-4-acetoxy-2-(((4S,5S)-5-(3-(benzylcarbamoyl)phenyl)-2,2-dimethyl-1,3-dioxolan-4-yl)methyl)-6-((R,2R)-1,2-diacetoxy-3-(3,5-dichloro-4-hydroxybenzamido)propyl)tetrahydro-2H-pyran-2-carboxylic acid (198, 9.00 mg, 0.010 mmol) in MeOH (0.180 mL) was added 1 M aq. NaOH (0.097 mL, 0.097 mmol). The reaction mixture was stirred for 2.5 h, after which time it was quenched with 1 N HCl (0.097 mL, 0.097 mmol) and purified directly by HPLC to provide A-321 (3.6 mg, 0.004 mmol, 46%) (MW_{Calc}+H=805.22; MW_{Obs}=805.44; 1HNMR=Y) and A-322 (1.7 mg, 0.002 mmol, 22% overall yield) (MW_{Calc}+H=805.22; MW_{Ob}S=805.20).

### Preparation of A-323

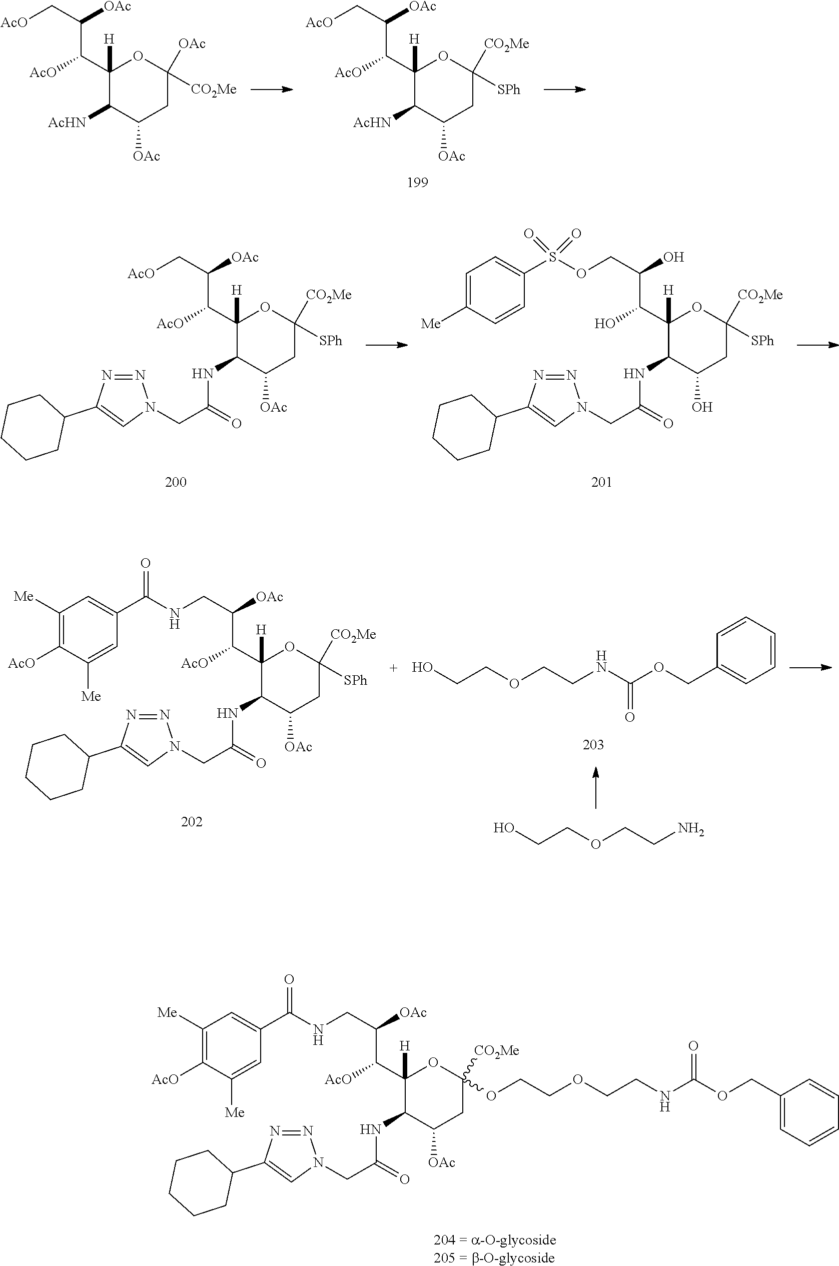

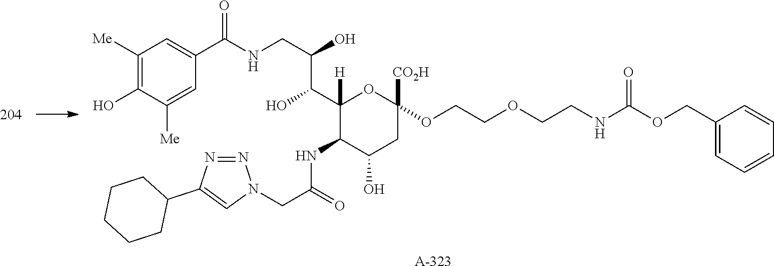 [SMILES: Cc1cc(C(=O)NC[C@@H](O)[C@@H](O)[C@@H]2O[C@@](OCCOCCNC(=O)OCc3ccccc3)(C(=O)O)C[C@H](O)[C@H]2NC(=O)Cn2cc(C3CCCCC3)nn2)cc(C)c1O ; "A-323"]

To a stirred solution of (1S,2R)-1-((2R,3R,4S)-3-acetamido-4,6-diacetoxy-6-(methoxycarbonyl)-tetrahydro-2H-pyran-2-yl)propane-1,2,3-triyl triacetate (20 g, 37.49 mmol) in DCM (68.9 mL) at was added phenylmercaptan (9.62 mL, 93.724 mmol) followed by a dropwise addition of BF₃·OEt₂ (23.75 mL, 187.449 mmol) over a 5-min period. The reaction was allowed to warm to room temperature and stirred for 16 h. An additional amount of BF₃·OEt₂ (2.375 ml, 18.745 mmol) was added, and the mixture was allowed to stir for 24 h. The completed reaction was cooled to 0° C., stirred for 10 min, and then quenched with sodium bicarbonate (787 g, 749.794 mmol) added piecemeal maintain the temperature below 10° C. The resultant mixture was stirred at 0° C. for 10 min, and then extracted with ethyl acetate (2×750 mL ea). The combined organic layers were washed with half sat. brine (300 mL), dried over Na₂SO₄, and concentrated. The residue was dissolved in EtOAc (100 mL), purified over a pad of silica gel (100 g) eluting with EtOAC (1.0 L), and the combined desired fractions were concentrated and dried under to provide compound 199 (21.8 g, 37.4 mmol, 99%).

To a stirred solution of (1S,2R)-1-((2R,3R,4S)-3-acetamido-4-acetoxy-6-(methoxycarbonyl)-6-(phenylthio)tetrahydro-2H-pyran-2-yl)propane-1,2,3-triyl triacetate (199, 10.03 g, 17.186 mmol) in methanol (100 mL) at room temperature was added methanesulfonic acid (4 mL, 61.599 mmol). The reaction mixture was warmed to 65° C. and stirred for 21 h, and then cooled to room temperature and stirred for 24 h. The resultant mixture was quenched with triethylamine (11.98 mL, 85.931 mmol), concentrated, and azeotroped to dryness with MeCN (3×100 mL ea) to provide the crude deacetylated product that was used directly in the next reaction without further purification.

To a stirred solution of crude (4S,5R,6R)-methyl 5-amino-4-hydroxy-2-(phenylthio)-6-((1R,2R)-1,2,3-trihydroxypropyl)tetrahydro-2H-pyran-2-carboxylate (199, 12.5 g, 33.474 mmol) in acetonitrile (100 mL) at 0° C. was added 2-(4-cyclohexyl-1H-1,2,3-triazol-1-yl)acetic acid (7.00 g, 33.474 mmol) followed by triethylamine (23.33 mL, 167.371 mmol) followed by HATU (15.91 g, 41.843 mmol). It was stirred at 0° C. for 2 h, after which time additional HATU (0.636 g, 1.674 mmol) was added, and stirred at 0° C. for 30 min, warm to room temperature, and stirred for 16 h, then slowly warmed up to RT overnight.

The resulting mixture was cooled to 0° C., followed by the addition of DMAP (1.022 g, 8.369 mmol), triethylamine (37.3 mL, 267.794 mmol), and finally a dropwise addition of Ac₂O (17.37 mL, 184.0 mmol). The final reaction mixture was stirred at 0° C. for 16 h, after which time it was slowly quenched with a slow addition of sat. sodium bicarbonate (350 mL) at 0° C. The resultant mixture was extracted with EtOAc (2×350 mL ea), and the organic layers were washed with half sat. brine (100 mL), dried over Na₂SO₄, filtered and concentrated. The resultant residue was purified over a Biotage Ultra SNAP column (100 g) eluting with 1% MeOH in DCM (3CV) followed by a gradient of 1%-10% MeOH in DCM (10 CV). The desired fractions were combined, concentrated and dried under vacuum to provide compound 200 (24.18 g, 33.0 mmol, 99%).

To a stirred solution of (1S,2R)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2,3-triyl triacetate (200, 24.53 g, 33.474 mmol) in methanol (156 mL) at 0° C. was slowly added 4.38 M sodium methoxide in methanol (7.65 mL, 33.474 mmol). The reaction mixture was stirred at 0° C. for 16 h, after which time pyridine (5.41 ml, 66.949 mmol) was added followed by a slow addition of conc. HCl (3.49 ml, 41.843 mmol) maintaining the temperature at approximately 0° C. The resultant mixture was warmed to room temperature, concentrated, and azeotroped to dry with MeCN (3×60 mL ea). The crude residue was dissolved in pyridine (153 mL), cooled to 0° C. followed by the addition p-toluenesulfonyl chloride (6.60 g, 34.62 mmol). The final reaction mixture was stirred for 2.5 h, after which time methanol (2.71 ml, 66.949 mmol) was added followed by stirring for 15 min. The mixture was concentrated, azeotroped to dry with n-heptane (2×100 mL ea). The resultant residue was purified over a Biotage Ultra SNAP column (100 g) eluting with 1% MeOH in DCM (3CV) followed by a gradient of 1%-10% MeOH in DCM (10 CV). The desired fractions were combined, concentrated and dried under vacuum to provide compound 201 (15.38 g, 21.4 mmol, 21%) (MW_{Calc}+H=719.23; MW_{Obs}=719.13).

To a stirred solution of (4S,5R,6R)-methyl 5-(2-(4-cyclohexyl-1H-1,2,3-triazol-1-yl)acetamido)-6-((1R,2R)-1,2-dihydroxy-3-(tosyloxy)propyl)-4-hydroxy-2-(phenylthio)tetrahydro-2H-pyran-2-carboxylate (201, 15.38 g, 21.396 mmol) in acetone (236 mL) and water (26.1 mL) at room temperature was added sodium azide (3.48 g, 53.489 mmol). The reaction mixture was heated to 73° C., stirred 24 h, after which time it was cooled to room temperature, concentrated, azeotroped to dry with MeCN (3×50 mL ea). The crude residue was used in the next reaction. (MW_{Calc}+H=590.23; MW_{Obs}=590.36).

To a stirred solution of crude (4S,5R,6R)-methyl 6-((1R,2R)-3-azido-1,2-dihydroxypropyl)-5-(2-(4-cyclohexyl-1H-1,2,3-triazol-1-yl)acetamido)-4-hydroxy-2-(phenylthio)tetrahydro-2H-pyran-2-carboxylate (12.62 g, 21.402 mmol) in DMF (124 mL) at 0° C. was added imidazole (14.57 g, 214.02 mmol) 1605.152 mmol) followed by triethylchlorosilane (18.12 ml, 107.01 mmol) and stirring at 0° C. for 1 h. The reaction mixture was warmed to room temperature for 24 h, after which time methanol (2.60 mL) was added. The mixture was cooled to 0° C. followed by the addition of water (248 mL), and then extraction with MTBE (2×250 mL ea). The combined organic layers were washed with sat. NaHCO₃ (50 mL), half sat. brine (50 mL), dried over Na₂SO₄, filtered, and concentrated to provide the crude persilylated intermediate (19.93 g, 21.37 mmol, 100%), which was used directly in the next step without purification.

To a stirred solution of crude (4S,5S,6R)-methyl 6-((5S,6R)-6-(azidomethyl)-3,3,8,8-tetraethyl-4,7-dioxa-3,8-disiladecan-5-yl)-5-(2-(4-cyclohexyl-1H-1,2,3-triazol-1-yl)acetamido)-2-(phenylthio)-4-((triethylsilyl)oxy)tetrahydro-2H-pyran-2-carboxylate (18.22 g, 19.54 mmol) in THF (208 mL) and water (13.85 mL) at 0° C. was added 1 M trimethylphosphine in THF (60 mL, 60.00 mmol). The reaction mixture was stirred at 0° C. for 3 h, after which time it was warmed to room temperature and stirred for 72 h. The completed reaction was concentrated, and azeotroped to dry with MeCN (3×50 mL ea). The crude residue was used directly in the next step without purification. (MW_{Calc}+H=906.50; MW_{Obs}=906.41).

To a stirred solution of crude (4S,5S,6R)-methyl 6-((5S,6R)-6-(aminomethyl)-3,3,8,8-tetraethyl-4,7-dioxa-3,8-disiladecan-5-yl)-5-(2-(4-cyclohexyl-1H-1,2,3-triazol-1-yl)acetamido)-2-(phenylthio)-4-((triethylsilyl)oxy)tetrahydro-2H-pyran-2-carboxylate (17.71 g, 19.538 mmol) in DMF (152 mL) was added 4-hydroxy-3,5-dimethylbenzoic acid (4.06 g, 24.422 mmol) and HOBT (2.99 g, 19.538 mmol). The mixture was cooled to 0° C. followed by the addition of triethylamine (10.89 ml, 78.151 mmol) and EDC (7.49 g, 39.076 mmol). The reaction mixture was stirred at 0° C. for 1.5 h, after which additional 4-hydroxy-3,5-dimethylbenzoic acid (1.0 g, 6.01 mmol), EDC (3.75 g, 19.54 mmol), and triethylamine (4 ml, 28.70 mmol) at 0° C. followed by stirring for 2 h. The reaction was quenched with water (304 mL), extracted with EtOAc (2×500 mL ea), and the combined organic layers were washed with sat. NaHCO₃ (100 mL), half sat. brine (100 mL), dried over Na₂SO₄, filtered, concentrated, and azeotroped to dry with MeOH (1×100 mL). The crude residue was used directly in the next step without purification.

To a stirred solution of crude (4S,5S,6R)-methyl 5-(2-(4-cyclohexyl-1H-1,2,3-triazol-1-yl)acetamido)-2-(phenylthio)-6-((5S,6R)-3,3,8,8-tetraethyl-6-((4-hydroxy-3,5-dimethylbenzamido)methyl)-4,7-dioxa-3,8-disiladecan-5-yl)-4-((triethylsilyl)oxy)tetrahydro-2H-pyran-2-carboxylate (20.60 g, 19.533 mmol) in methanol (258 mL) at 0° C. was added p-toluenesulfonic acid monohydrate (4.64 g, 24.417 mmol). The reaction mixture was stirred at 0° C. for 7 h, after which time triethylamine (8.17 ml, 58.60 mmol) was added, stirred for 5 min, then concentrated on rotavap, azeotroped to dryness with MeCN (3×50 mL ea). The crude residue was used directly in the next step without purification. (MW_{Calc}+H=712.29; MW_{Obs}=712.20).

To a stirred solution of crude (4S,5R,6R)-methyl 5-(2-(4-cyclohexyl-1H-1,2,3-triazol-1-yl)acetamido)-6-((1R,2R)-1,2-dihydroxy-3-(4-hydroxy-3,5-dimethylbenzamido)propyl)-4-hydroxy-2-(phenylthio)-tetrahydro-2H-pyran-2-carboxylate (13.90 g, 19.527 mmol) in acetonitrile (100 mL) at 0° C. was added DMAP (1.359 g, 11.12 mmol), and triethylamine (33 ml, 236.763 mmol) followed by a dropwise addition of Ac₂O (11.5 ml, 121.884 mmol) keeping the temperature approximately 0° C. The reaction mixture was stirred at 0° C. for 16 h, after which time it was slowly quenched with the addition of sodium bicarbonate (308 g, 292.909 mmol) at 0° C., allowed to stir for an additional 10 min followed by extraction with EtOAc (2×100 mL ea). The combined organic layers were washed with half sat. brine (50 mL), dried over Na₂SO₄, filtered, concentrated, and purified over a Biotage Ultra SNAP column (100 g) eluting with 1% MeOH in DCM (3CV) followed by a gradient of 1%-10% MeOH in DCM (10 CV). The desired fractions were combined, concentrated and dried under vacuum to provide compound 202 (4.0 g, 4.55 mmol, 23% from compound 201) (MW_{Calc}+H=880.34; MW_{Obs}=880.54).

To a stirred solution of 2-(2-aminoethoxy)ethanol (2.0 g, 19.02 mmol) in 1,4-dioxane (20 mL) and water (10 mL) was added solid sodium bicarbonate (2.397 g, 28.535 mmol) maintaining the temperature between 1° and 20° C., followed by a portion wise addition of CBZ-C1 (2.72 mL, 19.023 mmol). The reaction was warmed to room temperature, stirred for 3 h, after which time the completed reaction was diluted with water (20 mL) and EtOAc (20 mL). The layers were separated, and the aqueous layer was extracted with EtOAc (20 mL). The combined organic layers were washed with sat. NaHCO₃ (10 mL), half sat. brine (10 mL), dried over Na₂SO₄, filtered and concentrated. The resulting residue was purified over a Biotage Ultra SNAP column (25 g) eluting with 1% MeOH in DCM (3CV) followed by a gradient of 1%-10% MeOH in DCM (10 CV). The desired fractions were combined, concentrated and dried under vacuum to provide compound 203 (2.98 g, 12.45 mmol, 66%) (MW_{Calc}+Na=262.12; MW_{Obs}=262.27).

To a stirred solution of (1R,2R)-3-(4-acetoxy-3,5-dimethylbenzamido)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2-diyl diacetate (202), 35.6 mg, 0.04 mmol) and benzyl (2-(2-hydroxyethoxy)-ethyl)carbamate (203, 38.7 mg, 0.162 mmol) in DCE (0.50 mL) and acetonitrile (0.50 mL) at room temperature was added 200 mg of activated 4 Å molecular sieves followed by stirring for 1 h. The suspension was cooled to −35° C. followed by the addition of N-iodosuccinimide (32.6 mg, 0.145 mmol) in acetonitrile (0.326 mL), which was previously dried over 4 Å molecular sieves, and then trifluoromethanesulfonic acid (16.28 μl, 0.183 mmol). The reaction mixture was stirred at −25° C. for 30 min, after which time it was quenched with 1:1 ratio of sat. NaHCO₃:15 wt % Na₂S₂O₃ (5 mL), stirred for 10 min at room temperature, filtered, eluting with EtOAc (2×2 mL ea). The layers were separated, and the aqueous layer was extracted with EtOAc (2×3 mL ea). The combined organic layers were washed with sat. brine (3 mL), dried over Na₂SO₄, filtered, and concentrated. The residue was purified over a Biotage Ultra SNAP column (10 g) eluting with 1% MeOH in DCM (3CV) followed by a gradient of 1%-10% MeOH in DCM (10 CV). The impure products were then re-purified via HPLC to provide compound 204 (19.7 mg, 0.020 mmol, 43%, as the α-O-glycoside via 1H-NMR) (MW_{Calc}+H=1009.43; MW_{Obs}=1009.68) and 205 (6.7 mg, 0.007 mmol, 16%, as the β-O-glycoside via 1H-NMR) (MW_{Calc}+H=1009.43; MW_{Obs}=1009.68) after the desired fractions were combined, concentrated and dried under vacuum.

To a stirred solution (1R,2R)-3-(4-acetoxy-3,5-dimethylbenzamido)-1-((2R,3R,4S,6R)-4-acetoxy-6-(2-(2-(((benzyloxy)carbonyl)amino)ethoxy)ethoxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (204, 19.7 mg, 0.02 mmol) in methanol (0.80 mL) and water (0.30 mL) at room temperature was added 1 M aqueous NaOH (195 μl, 0.195 mmol). The reaction mixture was stirred for 3.5 h, after which time 1 N HCl (97 μL, 0.097 mmol) was added with stirring. The mixture was purified directly by HPLC to provide A-323 (14.4 mg, 0.017 mmol, 89%) (MW_{Calc}+H=827.38; MW_{Obs}=827.6) after the desired fractions were combined, concentrated and dried under vacuum.

### Preparation of A-324 and A-327

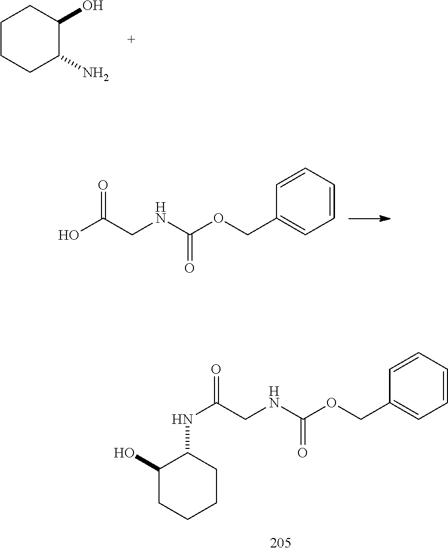

To a stirred solution (1R,2R)-2-aminocyclohexanol (259.5 mg, 2.253 mmol) and 2-(((benzyloxy)carbonyl)amino)acetic acid (471 mg, 2.253 mmol) in DMF (4 mL) at 0° C. was added triethylamine (0.628 ml, 4.506 mmol) followed by HATU (1071 mg, 2.816 mmol). The reaction mixture was stirred at 0° C. for 5 h, after which time the completed reaction was diluted with water (10 mL) and EtOAc. The layers were separated, and the aqueous layer was extracted with EtOAc (10 mL). The combined organic layers were washed with sat. NaHCO₃ (5 mL), half sat. brine (5 mL), and dried over Na₂SO₄ filtered and concentrated. The residue was purified over a Biotage Ultra SNAP column (10 g) eluting with 1 CV 5% EtOAC in heptane, 8 CV gradient of 5 to 100% EtOAC in heptane, 2 CV EtOAc, and a 6 CV gradient of 0 to 40% MeOH in EtOAc to provide compound 205 (500.5 mg, 1.634 mmol, 73%) after concentration of the desired fractions and drying under vacuum.

A-324 was prepared in a similar manner to A-323 starting with (1R,2R)-3-(4-acetoxy-3,5-dimethylbenzamido)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2-diyl diacetate (202, 42.2 mg, 0.048 mmol) and benzyl (2-(((1R,2R)-2-hydroxycyclohexyl)amino)-2-oxoethyl)carbamate (205, 51 mg, 0.166 mmol) to provide A-324 (3.5 mg, 0.004 mmol, 8% overall yield) (MW_{Calc}+H=894.42; MW_{Obs}=894.7).

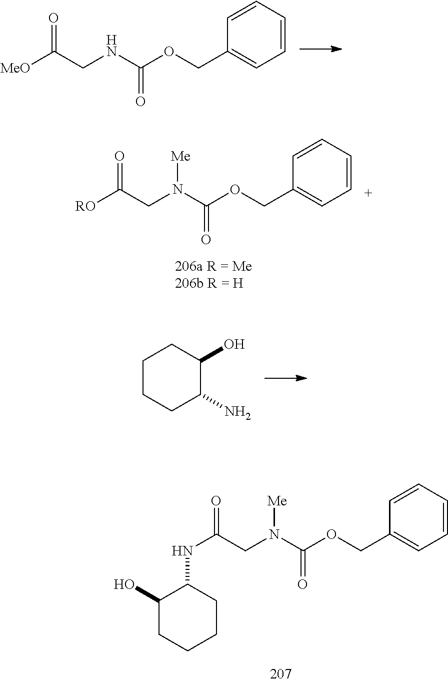

To a stirred solution of commercially available 2-(((benzyloxy)carbonyl)amino)acetic acid (2.00 g, 9.56 mmol) in DMF (10 mL) at 0° C. was slowly added 60% sodium hydride (2.658 g, 25.08 mmol) followed by methyl iodide (2.2 mL, 35.22 mmol) portion wise. The reaction mixture became turbid, thus additional DMF (10 mL) was added with stirring. The reaction mixture was stirred for 2 h at room temperature, after which time additional 60% sodium hydride (0.76 g, 19.21 mmol) was added portion wise followed by the slow addition of methyl iodide (1.20 mL, 19.12 mmol). The final reaction mixture was stirred for 16 h, after which time it was quenched with a slow addition of water (25 mL) and extracted with EtOAc (2×25 mL ea). The combined organic layers were washed with sat. NaHCO₃ (25 mL), a 1:1 ratio of water:brine (25 mL), dried over Na₂SO₄, filtered, concentrated, and dried. The residue was purified over a Biotage Ultra HP column (25 g) eluting with 1 CV 5% EtOAC in heptane, 8 CV gradient of 5 to 100% EtOAC in heptane, 1 CV EtOAc, and a 4 CV gradient of 0 to 40% MeOH in EtOAc to provide compound 206a (MW_{Calc}+Na=260.10; MW_{Obs}=260.25) after concentration of the desired fractions and drying under vacuum.

To a stirred solution of methyl 2-(((benzyloxy)carbonyl)(methyl)amino)acetate (206a, 1.00 g, 4.22 mmol) in THF (6.0 mL) at 0° C. was added 1 M NaOH is water (5.06 mL, 5.06 mmol) after which time the reaction mixture was warmed to room temperature and allowed to stir for 16 hours. The completed reaction was cooled to 0° C., and slowly acidified with 1 N HCl in water (5.46 mL, 5.46 mmol). The mixture concentrated and azeotroped to dry with acetonitrile (3×10 mL ea) to provide crude compound 206b as a colorless oil, which was used in the next reaction without further purification.

Compound 207 was prepared in a similar fashion to compound 205 starting with 2-(((benzyloxy)carbonyl) (methyl)amino)acetic acid (206, 0.87 g, 3.897 mmol) and (1R,2R)-2-aminocyclohexanol (1.347 g, 11.692 mmol) to provide compound 207 (1.200 g, 3.746 mmol, 94%) (MW_{Calc}+Na=343.17; MW_{Obs}=343.25).

A-325 was prepared in a similar manner to A-323 starting with (1R,2R)-3-(4-acetoxy-3,5-dimethylbenzamido)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2-diyl diacetate (200, 57.7 mg, 0.066 mmol) and benzyl (2-(((1R,2R)-2-hydroxycyclohexyl)amino)-2-oxoethyl)(methyl)carbamate (207, 123 mg, 0.154 mmol) to provide A-325 (1.6 mg, 0.002 mmol, 3% overall yield). (MW_{Calc}+H=908.44; MW_{Obs}=908.7

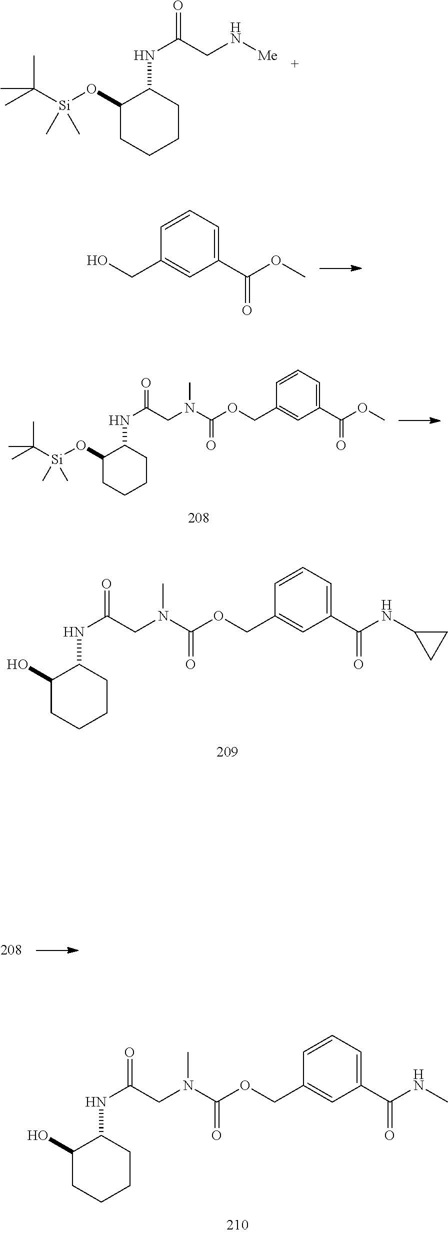

To a stirred solution of methyl 3-(hydroxymethyl)benzoate (0.930 g, 5.261 mmol) in DCE (9 mL) at 0° C. was added dried 4A molecular sieves (1.2 g) and triethylamine (1.741 ml, 12.489 mmol) followed by 4-Nitrophenyl chloroformate (1.019 g, 5.054 mmol). The mixture was diluted with DCE (6 mL), warmed to room temperature, and stirred for 16 h. The intermediate reaction was cooled to 0° C., followed by the dropwise addition of a solution of N-((1R,2R)-2-((tert-butyldimethylsilyl)-oxy)cyclohexyl)-2-(methylamino)acetamide (1.251 g, 4.163 mmol) in DCE (16 mL) maintaining the temperature. The final reaction mixture was stirred at 0° C. for 30 min, warmed to room temperature, and stirred for 2 h. The reaction was found to be incomplete thus the reaction mixture was cooled to 0° C., followed by the addition of pyridine (1.683 mL, 20.814 mmol), followed by the dropwise addition of another batch of the reaction intermediate formed above: methyl 3-(hydroxymethyl)benzoate (0.930 g, 5.261 mmol) in DCE (9 mL) at 0° C. was added dried 4A molecular sieves (1.2 g) and triethylamine (1.741 ml, 12.489 mmol) followed by 4-Nitrophenyl chloroformate (1.019 g, 5.054 mmol). The reaction mixture was warmed to room temperature and stirred for 16 h. This process was repeated one additional time, after which time the final reaction was quenched with sat. NaHCO₃ (10 mL), extracted with EtOAc (3×10 mL ea), and the combined organic layers were washed with sat. NaHCO₃ (10 mL), half sat. brine (10 mL), dried over Na2SO4, filtered, and concentrated. Biotage purification afforded 1.57g desired product. The residue was purified over a Biotage Ultra SNAP column (100 g) eluting with 2 CV 5% EtOAC in heptane, a 10 CV gradient of 5 to 100% EtOAC in heptane to provide compound 208 (1.57 g mg, 3.19 mmol, 77%) (MW_{Calc}+Na=515.27; MW_{Obs}=515.29) after concentration of the desired fractions and drying under vacuum.

To a stirred solution of methyl 3-((((2-(((1R,2R)-2-((tert-butyldimethylsilyl)oxy)cyclohexyl)amino)-2-oxoethyl)(methyl)carbamoyl)oxy)methyl)benzoate (208, 1.55 g, 3.146 mmol) in THF (18 mL) and water (6 mL) at was added lithium hydroxide (0.151 g, 6.292 mmol). The reaction mixture was stirred at for 1 h, warmed to RT and diluted with water (3 mL), and THF (12 mL), and stirred for 16 h. The reaction was found to be incomplete, thus added lithium hydroxide (41.5 mg, 1.733 mmol), and stirred for 16 h. The completed reaction cooled to 0° C., neutralized with 1 N HCl (12.58 ml, 12.584 mmol), and diluted with EtOAc (50 mL). The layers were separated, the aqueous layer was extracted with EtOAc (20 mL), and the combined organic layers were washed with pH 3-4 water (20 mL), half sat. brine (20 mL), dried over Na₂SO₄, filtered, concentrated, and dried under vacuum to provide the crude acid intermediate (1.50 g, 3.53 mmol, 100%), which was used directly without purification. (MW_{Calc}+H=479.25; MW_{Obs}=479.35).

To a stirred solution of 3-((((2-(((1R,2R)-2-((tert-butyldimethylsilyl)oxy)cyclohexyl)amino)-2-oxoethyl)(methyl)carbamoyl)oxy)methyl)benzoic acid (169 mg, 0.353 mmol) in acetonitrile (2.0 mL) at 0° C. was added DMAP (4.31 mg, 0.035 mmol), triethylamine (197 μl, 1.412 mmol) followed by cyclopropylamine (124 μl, 1.765 mmol) and HATU (403 mg, 1.059 mmol). The reaction was warmed to room temperature and stirred for 3 h. The completed reaction was diluted with EtOAc (5 mL). The layers were separated, the aqueous layer was extracted with EtOAc (2 mL), and the combined organic layers were washed with sat. NaHCO₃ (2 mL), half sat. brine (2 mL), dried over Na₂SO₄, filtered, concentrated, and dried under vacuum to provide the crude amide intermediate (183 mg, 0.353 mmol, 100%) (MW_{Calc}+H=518.30; MW_{Obs}=518.41), which was used directly without purification.

To a stirred solution of 3-(cyclopropylcarbamoyl)benzyl (2-(((1R,2R)-2-((tert-butyldimethylsilyl)-oxy)cyclohexyl)amino)-2-oxoethyl)(methyl)carbamate (183 mg, 0.353 mmol) in THF (3 mL) at 0° C. was added TBAF (1.060 mL, 1.06 mmol), then warmed at room temperature, and stirred for 16 h. The incomplete reaction was concentrated, azeotroped to dry with methanol (5 mL) followed by adding methanol (3 mL), cooling to 0° C., adding p-toluenesulfonic acid monohydrate (36 mg, 0.189 mmol) at 0° C. and stirring for 16 h. Additional p-toluenesulfonic acid monohydrate (300 mg, 1.577 mmol) was added at 0° C., and the final mixture was stirred for 4 h. The reaction was quenched with sat.

NaHCO₃ (10 mL), extracted with EtOAc (3×5 mL ea), and the and the combined organic layers were washed with half sat. brine (5 mL), dried over Na₂SO₄, filtered, concentrated. The residue was purified over a Biotage Ultra SNAP column (10 g) eluting with 2 CV 5% EtOAC in heptane, a 10 CV gradient of 5 to 100% EtOAC in heptane, and a 6 CV gradient of 0 to 40% methanol in EtOAc to provide compound 209 (92.8 mg, 0.230 mmol, 65% overall yield) (MW_{Calc}+Na=426.21; MW_{Obs}=426.35) after concentration of the desired fractions and drying under vacuum.

Compound 210 was prepared in a similar fashion to compound 209 starting with 3-((((2-(((1R,2R)-2-((tert-butyldimethylsilyl)oxy)cyclohexyl)amino)-2-oxoethyl)(methyl)-carbamoyl)oxy)methyl)benzoic acid 208 (169 mg, 0.353 mmol) and methylamine (2.00 mL, 4.00 mmol) to provide compound 210 (77.8 mg, 0.206 mmol, 34%) (MW_{Calc}+Na=400.23; MW_{Obs}=400.25).

A-326 was prepared in a similar manner to A-323 starting with (1R,2R)-3-(4-acetoxy-3,5-dimethylbenzamido)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2-diyl diacetate (200, 50 mg, 0.057 mmol) and 3-(cyclopropylcarbamoyl)benzyl (2-(((1R,2R)-2-hydroxycyclohexyl)amino)-2-oxoethyl)(methyl)carbamate (209, 92.8 mg, 0.23 mmol) to provide A-326 (1.4 mg, 0.001 mmol, 2% overall yield) (MW_{Calc}+H=991.47; MW_{Obs}=991.6).

A-327 was prepared in a similar manner to A-323 starting with (1R,2R)-3-(4-acetoxy-3,5-dimethylbenzamido)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2-diyl diacetate (200, 63 mg, 0.072 mmol) and 3-(methylcarbamoyl)benzyl (2-(((1R,2R)-2-hydroxycyclohexyl)amino)-2-oxoethyl)(methyl)carbamate (210, 77.8 mg, 0.206 mmol) to provide A-327 (1.5 mg, 0.002 mmol, 2% overall yield) (MW_{Calc}+H=965.46; MW_{Obs}=965.8).

### Preparation of A-328

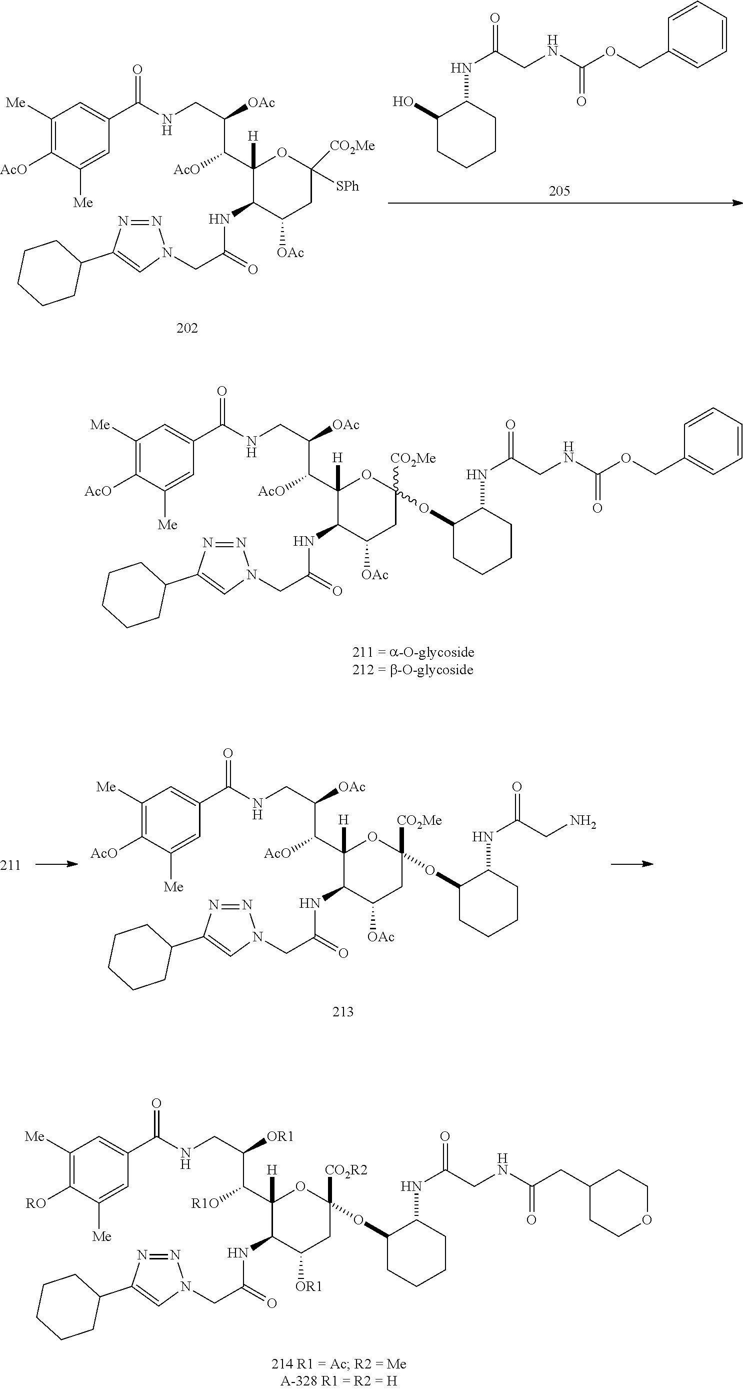

To a stirred solution of (1R,2R)-3-(4-acetoxy-3,5-dimethylbenzamido)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2-diyl diacetate (202, 42.2 mg, 0.048 mmol) and benzyl (2-(((1R,2R)-2-hydroxycyclohexyl)amino)-2-oxoethyl)carbamate (205, 51 mg, 0.166 mmol) in DCE (0.50 mL) and acetonitrile (0.50 mL) at room temperature was added 200 mg of activated 4 Å molecular sieves followed by stirring for 1 h. The suspension was cooled to −35° C. followed by the addition of N-iodosuccinimide (32.6 mg, 0.145 mmol) in acetonitrile (0.326 mL), which was previously dried over 4 Å molecular sieves, and then trifluoromethanesulfonic acid (16.28 μl, 0.183 mmol). The reaction mixture was stirred at −25° C. for 30 min, after which time it was quenched with 1:1 ratio of sat. NaHCO₃:15 wt % Na₂S₂O₃ (5 mL), stirred for 10 min at room temperature, filtered, eluting with EtOAc (2×2 mL ea). The layers were separated, and the aqueous layer was extracted with EtOAc (2×3 mL ea). The combined organic layers were washed with sat. brine (3 mL), dried over Na₂SO₄, filtered, and concentrated. The residue was purified over a Biotage Ultra SNAP column (10 g) eluting with 1% MeOH in DCM (3CV) followed by a gradient of 1%-10% MeOH in DCM (10 CV). The impure products were then re-purified via HPLC to provide compound 211 (7.4 mg, 0.009 mmol, 14%, as the (α-O-glycoside via 1H-NMR) (MW_{Calc}+Na=1098.47; MW_{Obs}=1098.56) and 212 (7.1 mg, 0.007 mmol, 14%, as the β—O-glycoside via 1H-NMR) (MW_{Calc}+Na=1098.47; MW_{Obs}=1098.62) after the desired fractions were combined, concentrated and dried under vacuum.

To a stirred solution of (1R,2R)-3-(4-acetoxy-3,5-dimethylbenzamido)-1-((2R,3R,4S,6R)-4-acetoxy-6-(((1R,2R)-2-(2-(((benzyloxy)carbonyl)amino)acetamido)-cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (212, 93 mg, 0.086 mmol) in THF (1.550 mL) was added water (400 μl, 22.203 mmol), and then 20% palladium hydroxide on carbon (24.27 mg, 0.017 mmol). The reaction suspension was purged with hydrogen gas (5×) and placed under a hydrogen atmosphere (balloon pressure) for 2 h, after which time the mixture was purged with nitrogen (5×) followed by the addition of THF (3 mL), palladium hydroxide on carbon (36.4 mg, 0.026 mmol), purged with hydrogen gas (5×), and placed under a hydrogen atmosphere (balloon pressure) for 16 h. The completed reaction was purged with nitrogen (5×) and filtered followed by filtered over a pad of Celite (5 g), eluting with acetonitrile (5×3 mL ea). The combined filtrate was concentrated to provide compound 213 (ca. 81 mg, 0.086 mmol, 100%) (MW_{Calc}+H=942.44; MW_{Obs}=942.45 and was used in the next reaction without further purification.

To a stirred solution of (1R,2R)-3-(4-acetoxy-3,5-dimethylbenzamido)-1-((2R,3R,4S,6R)-4-acetoxy-6-(((1R,2R)-2-(2-aminoacetamido)cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (213, 10 mg, 0.011 mmol) in acetonitrile (2.0 mL) at room temperature was added tetrahydropyran-4-acetic acid (9.18 mg, 0.064 mmol) and triethylamine (0.018 ml, 0.127 mmol) followed by HATU (24.22 mg, 0.064 mmol). The reaction was stirred for 16 h, after which time the completed reaction was diluted with water (3 mL), and extracted with EtOAc (3×2 mL ea). The combined organic layers were washed with sat. NaHCO₃ (3 mL), azeotroped to dry with methanol, and concentrated to provide crude compound 214.

To a stirred solution of 214 in MeOH (0.80 mL, 19.774 mmol) and water (0.40 mL), was added 1 M aqueous NaOH (0.212 ml, 0.212 mmol) at room temperature followed by stirring for 16 h. The completed reaction was neutralized with 1 N HCl (0.180 ml, 0.18 mmol) and purified directly by HPLC to provide A-328 (4.1 mg, 0.005 mmol, 42% overall yield) (MW_{Calc}+H=886.45; MW_{Obs}=886.6) after concentration of the desired fractions and drying under vacuum.

### Preparation of A-329

A-329 was prepared in a similar manner to A-328 starting with (1R,2R)-3-(4-acetoxy-3,5-dimethylbenzamido)-1-((2R,3R,4S,6R)-4-acetoxy-6-(((1R,2R)-2-(2-aminoacetamido)-cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (213, 10 mg, 0.011 mmol) and 2-(cyclopentyloxy)acetic acid (9.18 mg, 0.064 mmol) to provide A-329 (4.8 mg, 0.005 mmol, 49% overall yield) (MW_{Calc}+H=886.45; MW_{Obs}=886.6).

### Preparation of A-330

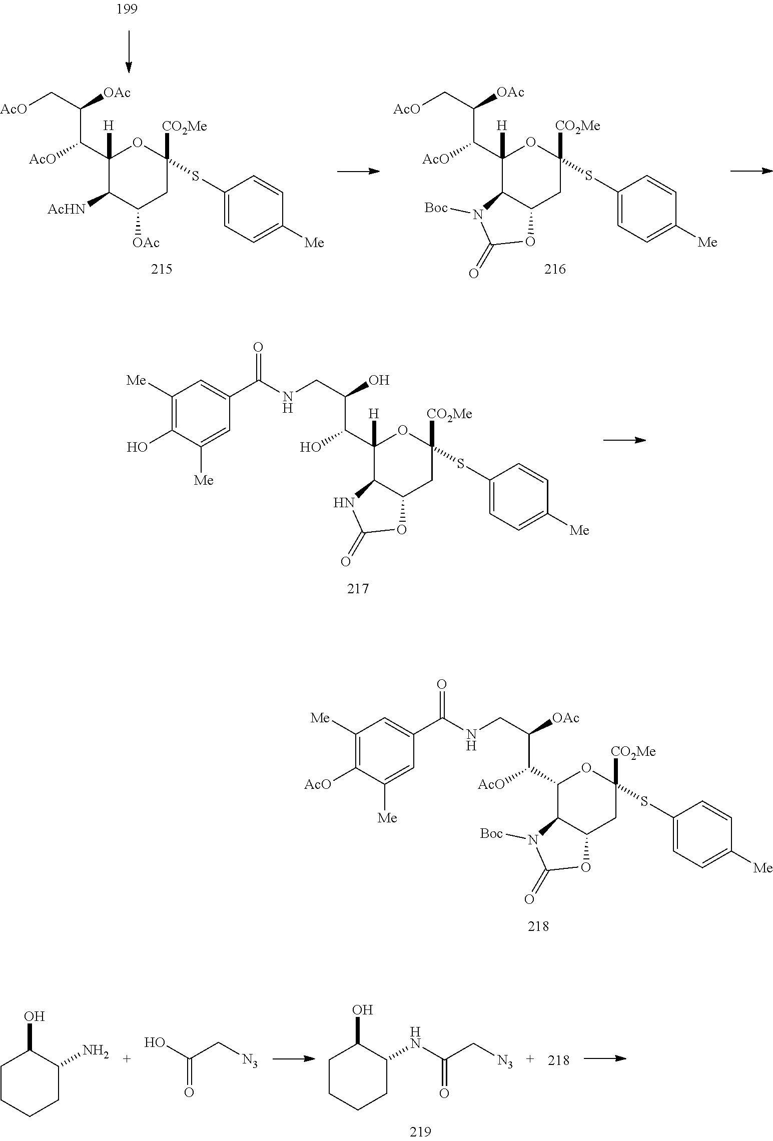

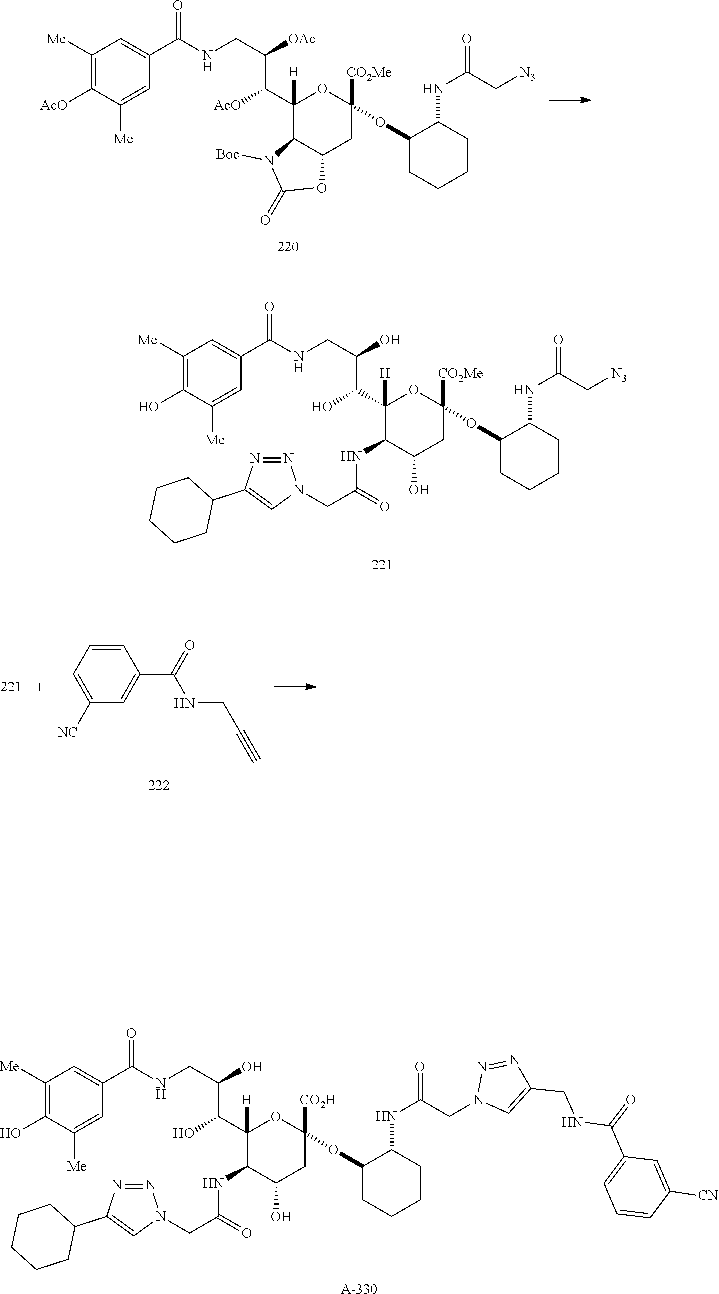

A solution of (2S,4S,5R,6R)-methyl 5-acetamido-2,4-dihydroxy-6-((R,2R)-1,2,3-trihydroxypropyl)tetrahydro-2H-pyran-2-carboxylate (199, 10.00 g. 25.054 mmol) in acetyl chloride (320 mL, 4.48 mol) at room temperature was warmed to 32° C. and stirred for 48 hours. The completed reaction was concentrated, azeotroped to dry with toluene (2×100 mL ea), followed by drying under high vacuum. The crude product was used in the next step without further purification. (MW_{Calc}+H=510.13; MW_{Obs}=510.20).

To a stirred solution of (1S,2R)-1-((2R,3R,44S,6R)-3-acetamido-4-acetoxy-6-chloro-6-(methoxycarbonyl)tetrahydro-2H-pyran-2-yl)propane-1,2,3-triyl triacetate (18.0 g, 35.30 mmol) in dichloromethane (270 mL) at 0° C. was added 4-methylbenzenethiol (13.15 g, 105.905 mmol) followed by Hunig's base (20.35 mL, 116.50 mmol). The reaction mixture was stirred at 0° C. for 1 h, after which time it was warmed to room temperature and stirred for an additional 14 h. The completed reaction was diluted with MTBE (350 mL), washed with saturated aqueous ammonium chloride (600 mL), and the aqueous layer was extracted with MTBE (150 mL). The combined organic layers were washed with sat. sodium bicarbonate (100 mL), the aqueous layer was back extracted with MTBE (150 mL), and the final combined organic layers were washed with brine (50 mL) and dried over magnesium sulfate. The dried organic layer was filtered, concentrated to dry to provide a crude solid. The solid was slurried in a mixture of heptane (300 mL) and ethyl acetate (20 mL) at 50° C. for 30 minutes, after which time it was cooled to 0° C. and allowed to stand for 30 minutes. The resulting solid was filtered and dried under high vacuum to provide 215 (17.10 g, 28.60 mmol, 81%) (MW_{Calc}+H=598.19; MW_{Obs}=598.13).

To a stirred solution of (1S,2R)-1-((2R,3R,44S,6S)-3-acetamido-4-acetoxy-6-(methoxycarbonyl)-6-(p-tolylthio)tetrahydro-2H-pyran-2-yl)propane-1,2,3-triyl triacetate (215, 20 g, 33.465 mmol) in methanol (220 mL) at room temperature was carefully added methanesulfonic acid (8.04 mL, 123.822 mmol). The reaction mixture was warmed to 65° C., and stirred for 24 h, after which time it was cooled to 0° C., and triethylamine (23.32 mL, 167.327 mmol) was slowly added. The quenched mixture was warmed to room temperature, stirred for an additional 15 min, concentrated, and dried under vacuum. The deacylated product was used in the next reaction without purification. (MW_{Calc}+H=388.14; MW_{Obs}=388.16).

To a stirred solution of (2S,4S,5R,6R)-methyl 5-amino-4-hydroxy-2-(p-tolylthio)-6-((1R,2R)-1,2,3-trihydroxypropyl)tetrahydro-2H-pyran-2-carboxylate (40 g, 25.81 mmol) in water (150 mL) and acetonitrile (150 mL) at room temperature was added sodium bicarbonate (10.84 g, 129.05 mmol), and then cooled to 0° C. 4-Nitrophenyl chloroformate (13.01 g, 64.525 mmol) in acetonitrile (150 ml, 2871.973 mmol) was added dropwise over 30 minutes maintaining the temperature between 0 to 5° C., after which time the reaction mixture was stirred for 2.5 hours at 0° C. The completed reaction was diluted with EtOAc (500 mL), and the layers separated. The aqueous layers were extracted with EtOAc (100 mL), and the combined organic layers were washed with brine (100 mL), dried over Na2SO4, filtered and concentrated to a provide the desired 4-5 urethane intermediate (28 g) as a yellow solid. (MW_{Calc}+Na=436.11; MW_{Obs}=436.12).

To a stirred solution of (3aR,44R,6S,7aS)-methyl 2-oxo-6-(p-tolylthio)-4-((1R,2R)-1,2,3-trihydroxypropyl)hexahydro-2H-pyrano[3,4-d]oxazole-6-carboxylate (27 g, 21.551 mmol) in pyridine (29.6 mL, 366.363 mmol) at room temperature under a N₂ atmosphere was added dropwise acetic anhydride (30.5 mL, 323.262 mmol) maintaining the temperature below 40° C. The reaction mixture was stirred at room temperature for 18 h, after which time it was diluted with EtOAc (500 mL) and a slow addition of 2 M HCl (500 mL). The layers were separated, and the aqueous layers was extracted with EtOAc (200 mL). The combined organic layers were washed with 1N HCL (100 mL), sat NH₄CL (100 mL) and brine (100 mL). The combined aqueous layers were extracted with EtOAc (300 mL) followed by this organic layer being washed with brine (50 mL). The combined organic layers were concentrated, and the residue was purified over a Biotage Ultra SNAP column (340 g) eluting with a 10 CV gradient of 0 to 100% EtOAc in heptane to provide compound the per-acetylated intermediate (11.6 g, 21.55 mmol, 100%) after the desired fractions were combined, concentrated and dried under vacuum. (MW_{Calc}+Na=562.11; MW_{Obs}=562.06).

To a stirred solution of (1S,2R)-1-((3aR,4R,6S,7aS)-6-(methoxycarbonyl)-2-oxo-6-(p-tolylthio)hexahydro-2H-pyrano[3,4-d]oxazol-4-yl)propane-1,2,3-triyl triacetate (0.900 g, 1.668 mmol) in THF (10 mL) at room temperature was added BOC-anhydride (0.546 g, 2.502 mmol), triethylamine (0.35 mL, 2.502 mmol) and DMAP (0.048 g, 0.334 mmol) followed by stirring for 30 min. The completed reaction diluted with ethyl acetate (20 mL), washed with sat. sodium bicarbonate, dried over Na₂SO₄, filtered, concentrated, and the residue was purified over a Biotage Ultra SNAP column (25 g) eluting with a 10 CV gradient of 0 to 15% EtOAc in heptane to provide compound 216 (1.00 g, 1.563 mmol, 94%) (MW_{Calc}+Na=662.20; MW_{Obs}=662.27) after the desired fractions were combined, concentrated and dried under vacuum.

To a stirred solution of (3aR,4R,6S,7aS)-3-tert-butyl 6-methyl 2-oxo-6-(p-tolylthio)-4-((1S,2R)-1,2,3-triacetoxypropyl)tetrahydro-2H-pyrano[3,4-d]oxazole-3,6(6H)-dicarboxylate (216, 4.85 g, 7.582 mmol) in methanol (80 mL) at 0° C. was slowly added 4.37 M sodium methoxide in methanol (5.20 mL, 22.746 mmol). The mixture was warmed room temperature and stirred for 30 min, after which time the reaction mixture was concentrated, and purified over a Biotage Ultra SNAP column (50 g) eluting with a 10 CV gradient of 0 to 15% MeOH in DCM to provide the deacylated intermediate (3.39 g, 6.95 mmol, 92% yield) (MW_{Calc}+H=488.19; MW_{Obs}=488.32) after the desired fractions were combined, concentrated and dried under vacuum.

To a stirred solution of (2S,4S,5R,6R)-methyl 5-((tert-butoxycarbonyl)amino)-4-hydroxy-2-(p-tolylthio)-6-((1R,2R)-1,2,3-trihydroxypropyl)tetrahydro-2H-pyran-2-carboxylate (3.39 g, 6.953 mmol) in pyridine (33.7 ml, 417.176 mmol) at 0° C. was added p-toluenesulfonyl chloride (1.299 g, 6.814 mmol). The reaction mixture was stirred at 0° C. for 1 hr, after which time additional p-toluene-sulfonyl chloride (0.133 g, 0.695 mmol) was added, and the reaction mixture was stirred at 0° C. for 1 hr. The mixture was diluted with MeOH (10 mL) and stirred at room temperature for 10 mins. The reaction mixture was concentrated, and purified over a Biotage Ultra SNAP column (50 g) eluting with a 10 CV gradient of 0 to 15% MeOH in DCM to provide the tosylated intermediate (4.1 g, 6.39 mmol, 92%) (MW_{Calc}+H=642.20; MW_{Obs}=642.41) after the desired fractions were combined, concentrated and dried under vacuum.

To a stirred solution of (2S,4S,5R,6R)-methyl 5-((tert-butoxycarbonyl)amino)-6-((1R,2R)-1,2-dihydroxy-3-(tosyloxy)propyl)-4-hydroxy-2-(p-tolylthio)tetrahydro-2H-pyran-2-carboxylate (4.2 g, 6.545 mmol) in acetone (72.1 mL) and water (7.99 mL) at room temperature was added sodium azide (2.127 g, 32.723 mmol). The reaction was warmed to 73° C., after which time it was stirred for 48 h. The completed reaction was concentrated, azeotroped to dry with MeCN (3×100 mL), and the resultant azide compound was used in the next step without further purification. (MW_{Calc}+H=513.19; MW_{Obs}=513.39).

To a stirred solution of crude (2S,4S,5R,6R)-methyl 6-((1R,2R)-3-azido-1,2-dihydroxypropyl)-5-((tert-butoxycarbonyl)amino)-4-hydroxy-2-(p-tolylthio)tetrahydro-2H-pyran-2-carboxylate (6.2 g, 12.096 mmol) in DMF (70.2 mL) at room temperature was added imidazole (8.23 g, 120.958 mmol). The mixture was cooled to 0° C. followed by the addition of triethylchlorosilane (10.24 mL, 60.479 mmol). The reaction mixture was stirred at 0° C. for 1 h, after which time it was warmed to room temperature, and stirred for 16 h. The completed reaction was diluted with methanol (1.468 mL), cooled down to 0° C., followed by the addition of water (0.218 mL). The mixture was extracted with MTBE (2×20 mL ea), and the combined organic layers were washed with sat. NaHCO₃ (5 mL) half sat. brine (5 mL), dried over Na₂SO₄, filtered, and concentrated to dryness to provide the per-silylated intermediate that was used in the next reaction without further purification. (MW_{Calc}+H=855.45; MW_{Obs}=855.72).

To a stirred solution of crude (2S,4S,5S,6R)-methyl 6-((5S,6R)-6-(azidomethyl)-3,3,8,8-tetraethyl-4,7-dioxa-3,8-disiladecan-5-yl)-5-((tert-butoxycarbonyl)amino)-2-(p-tolylthio)-4-((triethylsilyl)oxy)-tetrahydro-2H-pyran-2-carboxylate (16.3 g, 19.056 mmol) in THF (203 mL) and water (13.49 mL) at 0° C. was added trimethylphosphine (58.5 ml, 58.503 mmol). The reaction mixture was stirred at 0° C. for 3 h, then warmed to room temperature, and stirred for 16 h. The completed reaction mixture was concentrated, azeotroped to dryness with MeCN (3×50 mL ea) and used in the next reaction without further purification. (MW_{Calc}+H=829.46; MW_{Obs}=829.66).

To a stirred solution of crude (2S,4S,5S,6R)-methyl 6-((5S,6R)-6-(aminomethyl)-3,3,8,8-tetraethyl-4,7-dioxa-3,8-disiladecan-5-yl)-5-((tert-butoxycarbonyl)amino)-2-(p-tolylthio)-4-((triethylsilyl)oxy)-tetrahydro-2H-pyran-2-carboxylate (13.17 g, 15.88 mmol) in DMF (124 mL) at 0° C. was added 4-hydroxy-3,5-dimethylbenzoic acid (3.96 g, 23.82 mmol) and HOBT (2.432 g, 15.88 mmol) followed by triethylamine (8.85 mL, 63.519 mmol) and EDC (6.09 g, 31.759 mmol). The reaction mixture was stirred at 0° C. for 10 mins, warmed to room temperature, and stirred for 72 h. The completed reaction was cooled to 0° C., followed by the addition of water (50 mL). The final mixture was extracted with EtOAc (2×500 mL ea), and the combined organic layers were washed with sat. NaHCO₃ (100 mL), half sat. brine (100 mL), dried over Na₂SO₄, and concentrated to provide the acyl analog, which was used directly without further purification. (MW_{Calc}+H=977.52; MW_{Obs}=977.66).

(2S,4S,5S,6R)-methyl 5-((tert-butoxycarbonyl)amino)-6-((5S,6R)-3,3,8,8-tetraethyl-6-((4-hydroxy-3,5-dimethylbenzamido)methyl)-4,7-dioxa-3,8-disiladecan-5-yl)-2-(p-tolylthio)-4-((triethylsilyl)oxy)tetrahydro-2H-pyran-2-carboxylate (8.4 g, 8.593 mmol) at 0° C. was dissolved in 4 N HCl (2 mL, 65.824 mmol) in dioxane with stirring. The reaction was warmed to room temperature, and stirred for 1 hr. The completed reaction was concentrated, azeotroped to dry with acetonitrile (2×10 mL ea) and used in the next reaction without further purification. (MW_{Calc}+H=535.20; MW_{Obs}=535.36).

To a stirred solution of crude (2S,4S,5R,6R)-methyl 5-amino-6-((1R,2R)-1,2-dihydroxy-3-(4-hydroxy-3,5-dimethylbenzamido)propyl)-4-hydroxy-2-(p-tolylthio)tetrahydro-2H-pyran-2-carboxylate (4.51 g, 8.436 mmol) in water (49.1 mL) and acetonitrile (48.9 mL) was added sodium bicarbonate (3.54 g, 42.179 mmol), cooled to 0° C., after which time added dropwise 4-nitrophenyl chloroformate (3.40 g, 16.872 mmol) in acetonitrile (48.9 mL) over 5 min maintaining the temperature between 0 and 5° C. The reaction was stirred for 2.5 hours at 0° C. The completed reaction was diluted with EtOAc (200 mL), the layers separated, and the aqueous layer was extracted with EtOAc (100 mL). The combined organic layers were washed with NaHCO₃ (50 mL), dried over Na₂SO₄, filtered and concentrated. The residue was purified over a Biotage Ultra SNAP column (50 g) eluting with a 5 CV gradient of 0 to 40% EtOAc in heptane, then a 5 CV gradient of 40 to 80% EtOAc in heptane, to provide compound 217 (2.05 g, 3.66 mmol, 43% overall yield) (MW_{Calc}+H=561.18; MW_{Obs}=561.34) after the desired fractions were combined, concentrated and dried under vacuum.

To a stirred solution of (3aR,4R,6S,7aS)-methyl 4-((1R,2R)-1,2-dihydroxy-3-(4-hydroxy-3,5-dimethylbenzamido)propyl)-2-oxo-6-(p-tolylthio)hexahydro-2H-pyrano[3,4-d]oxazole-6-carboxylate 217 (1.72 g, 3.068 mmol) in DCM (99 mL) and pyridine (1.489 ml, 18.408 mmol) at 0° C. was added dropwise acetic anhydride (0.868 ml, 9.204 mmol). The reaction mixture was stirred 4 h at 0° C., after which time it was warmed to room temperature, and stirred overnight. The completed reaction mixture was washed with sat. NaHCO₃ (5 mL), and the organic layer was washed with brine (5 mL), dried over Na₂SO₄, filtered and concentrated to dryness to provide the crude acetylated intermediate (1.62 g, 2.359 mmol) that was used directly without further purification. (MW_{Calc}+H=687.21; MW_{Obs}=687.40).

To a stirred solution of crude (1R,2R)-3-(4-acetoxy-3,5-dimethylbenzamido)-1-((3aR,4R,6S,7aS)-6-(methoxycarbonyl)-2-oxo-6-(p-tolylthio)hexahydro-2H-pyrano[3,4-d]oxazol-4-yl)propane-1,2-diyl diacetate (1.62 g, 2.359 mmol) in THF (44.5 mL) at 0° C. was added Boc-anhydride (0.548 ml, 2.359 mmol) and DMAP (0.058 g, 0.472 mmol). The reaction was stirred at 0° C. for 30 min, after which time the completed reaction was concentrated, and the residue directly purified over a Biotage Ultra SNAP column (50 g) eluting with a 10 CV gradient of 0 to 40% EtOAc in heptane to provide compound 218 (960 mg, 1.220 mmol, 51.7% overall yield) (MW_{Calc}+H=787.27; MW_{Obs}=787.38) after the desired fractions were combined, concentrated and dried under vacuum.

To a stirred solution of 2-azidoacetic acid (2.01 g, 19.888 mmol) in DCM (192 mL) at 0° C. was added HOBT (3.65 g, 23.866 mmol) triethylamine (11.09 mL, 79.553 mmol) and EDC (7.63 g, 39.777 mmol) followed by stirring for 10 min, after which time (1R,2R)-2-aminocyclohexanol (2.405 g, 20.883 mmol) was added at 0° C. The reaction mixture was warmed to room temperature and stirred for 16 h. The reaction was diluted with 100 mL NaHCO₃, extracted with EtOAc (2×400 mL ea), and the combined organic layers were washed with sat. brine (50 mL), dried over Na₂SO₄, filtered and concentrated. The residue purified over a Biotage Ultra SNAP column (50 g) eluting with 5 CV EtOAc to provide compound 219 (3.12 g, 15.74 mmol, 79%) (MW_{Calc}+H=199.11; MW_{Obs}=199.07) after the desired fractions were combined, concentrated and dried under vacuum.

To a stirred solution of (3aR,4R,6S,7aS)-3-tert-butyl 6-methyl 4-((1R,2R)-1,2-diacetoxy-3-(4-acetoxy-3,5-dimethylbenzamido)propyl)-2-oxo-6-(p-tolylthio)tetrahydro-2H-pyrano[3,4-d]oxazole-3,6(6H)-dicarboxylate (218, 810 mg, 1.029 mmol) and 2-azido-N-((1R,2R)-2-hydroxycyclohexyl)-acetamide (219, 441 mg, 2.227 mmol) in DCM (10.6 mL) at room temperature was added dried 4A molecular sieves powder (2 g) followed by stirring for 16 h. The mixture was cooled to −40° C., after which time N-iodosuccinimide (625 mg, 2.779 mmol) was added to the mixture followed by the addition of trifluoromethanesulfonic acid (99 μL, 1.113 mmol). The final reaction mixture was stirred at −40° C. for 1.5 hr. The completed reaction was quenched with a mixture of 1:1 sat. NaHCO₃ to Na₂S₂O₃ (10 mL), extracted with EA (2×10 mL ea). The combined organic layers were washed with sat. brine (5 mL), dried over Na₂SO₄, filtered and concentrated. The residue purified over a Biotage Ultra SNAP column (50 g) eluting with a 10 CV gradient of 40 to 80% EtOAc in heptane to provide compound 220 (880 mg, 1.02 mmol, 99%) (MW_{Calc}+H=861.34; MW_{Obs}=861.43) of an anomeric mixture after the desired fractions were combined, concentrated and dried under vacuum.

To a stirred solution of an anomeric mixture of (3aR,4R,6R,7aS)-3-tert-butyl 6-methyl 6-(((1S,2R)-2-(2-azidoacetamido)-cyclohexyl)oxy)-4-((1R,2R)-1,2-diacetoxy-3-(4-acetoxy-3,5-dimethylbenzamido)propyl)-2-oxotetrahydro-2H-pyrano[3,4-d]oxazole-3,6(6H)-dicarboxylate (220, 880 mg, 1.02 mmol) in methanol (39.8 mL) at 0° C. was added 25% sodium methoxide in MeOH (0.964 mL, 4.217 mmol). The mixture was warmed to room temperature and stirred for 30 min, after which time DOWEX 50W×4, hydrogen form resin (4. 61 g, 200-400 mesh) was added to the reaction, and stirred for 10 min. The quenched mixture was filtered eluting with MeOH (2×10 mL ea), and the combined filtrate was concentrated to dryness, and used in the next step without further purification. (MW_{Calc}+Na=731.31; MW_{Obs}=731.23).

To a stirred solution of an anomeric mixture of (2RS, 4S,5R,6R)-methyl 2-(((1S,2R)-2-(2-azidoacetamido)cyclohexyl)oxy)-5-((tert-butoxycarbonyl)amino)-6-((1R,2R)-1,2-dihydroxy-3-(4-hydroxy-3,5-dimethylbenzamido)propyl)-4-hydroxytetrahydro-2H-pyran-2-carboxylate (593 mg, 0.836 mmol) in DCM (41.3 mL) at 0° C. was added pyridine (623 μL, 7.704 mmol) followed acetic anhydride (363 μl, 3.852 mmol). The reaction was stirred at 0° C. for 1 h, then warmed to room temperature and stirred for 16 h. The completed reaction mixture was washed with sat. NaHCO₃ (30 mL), and the aqueous layer was extracted with EtOAc (2×10 mL ea). The combined organic layers were washed with brine (10 mL), dried over Na2SO4, filtered and concentrated to dryness. The resultant crude product was used in the next step without further purification. (MW_{Calc}+H=877.38; MW_{Obs}=877.50).

To a stirred solution of an anomeric mixture of (1R,2R)-3-(4-acetoxy-3,5-dimethylbenzamido)-1-((2RS,3R,4S,6R)-4-acetoxy-6-(((1S,2R)-2-(2-azidoacetamido)cyclohexyl)oxy)-3-((tert-butoxycarbonyl)amino)-6-(methoxycarbonyl)tetrahydro-2H-pyran-2-yl)propane-1,2-diyl diacetate (733 mg, 0.836 mmol) in DCM (5.10 mL) at 0° C. was added dropwise TFA (5.17 mL, 67.111 mmol) over a 5 min period. The reaction was stirred at 0° C. for 1 h, then warmed to room temperature and stirred for 20 min. The completed reaction was concentrated, and azeotroped to dryness with Toluene (3×20 mL ea).

The resultant crude product was used in the next step without further purification. (MW_{Calc}+H=777.32; MW_{Obs}=777.44).

To a stirred solution of an anomeric mixture of (1R,2R)-3-(4-acetoxy-3,5-dimethylbenzamido)-1-((2RS,3R,4S,6R)-4-acetoxy-3-amino-6-(((1S,2R)-2-(2-azidoacetamido)cyclohexyl)oxy)-6-(methoxycarbonyl)tetrahydro-2H-pyran-2-yl)propane-1,2-diyl diacetate (649 mg, 0.836 mmol) and 2-(4-cyclohexyl-1H-1,2,3-triazol-1-yl)acetic acid (649 mg, 0.836 mmol) in DCM (24.4 mL) at 0° C. was added HATU (720 mg, 1.892 mmol) and triethylamine (879 μl, 6.308 mmol). The reaction mixture was stirred at 0° C. 10 mins, warmed to room temperature, and stirred for 72 h. The reaction was cooled 0° C., after which time water (50 mL) was added. The mixture was extracted with EtOAc (2×500 mL), and the combined organic layers were washed with sat. NaHCO₃ (50 mL), half sat. brine (50 mL), dried over Na₂SO₄, filtered, and concentrated to dryness. The residue purified over a Biotage Ultra SNAP column (50 g) eluting with 5 CV of 20% EtOAc in heptane. The desired fractions were concentrated, and the resultant mixture was purified by HPLC to provide compound 221 (394 mg, 0.407 mmol, 49% overall yield) (MW_{Calc}+H=968.43; MW_{Obs}=968.53), as a pure α-isomer. Approximately 10% of the β-isomer was also isolated.

To a stirred solution of (1R,2R)-3-(4-acetoxy-3,5-dimethylbenzamido)-1-((2R,3R,4S,6R)-4-acetoxy-6-(((1S,2R)-2-(2-azidoacetamido)cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (221, 10 mg, 0.010 mmol) and 3-cyano-N-(prop-2-yn-1-yl)benzamide (222, 5.71 mg, 0.031 mmol) in THF (296 μl, 3.616 mmol) was added tert-butanol (296 μl, 3.099 mmol) and water (298 μl, 16.529 mmol), it was then added copper(ii) sulfate pentahydrate (1.548 mg, 6.198 μmol) and L-ascorbic acid sodium salt (20.47 mg, 0.103 mmol) at 0° C. The reaction was stirred at 0° C. for 1 h, warmed room temperature, and stirred for 16 h. The completed reaction was taken to the next step without workup. (MW_{Calc}+H=1152.49; MW_{Obs}=1152.64).

To a stirred crude mixture of (1R,2R)-3-(4-acetoxy-3,5-dimethylbenzamido)-1-((2R,3R,4S,6R)-4-acetoxy-6-(((1R,2R)-2-(2-(4-((3-cyanobenzamido)methyl)-1H-1,2,3-triazol-1-yl)acetamido)cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (ca. 12 mg, 0.010 mmol) was added methanol (0.755 mL) followed by 1 M NaOH (135 μL, 0.135 mmol) at 0° C. The mixture was warmed to room temperature and stirred for 1 h. The completed reaction was neutralized with 1 N HCl (104 μL, 0.104 mmol) and directly purified by HPLC to provide A-330 (3.2 mg, 0.003 mmol, 32%) (MW_{Calc}+H=970.44; MW_{Obs}=970.7) after the desired fractions were combined, concentrated and dried under vacuum.

### Preparation of A-331 to A-334

A-331 was prepared in a similar manner to A-330 starting with (1R,2R)-3-(4-acetoxy-3,5-dimethylbenzamido)-1-((2R,3R,4S,6R)-4-acetoxy-6-(((1S,2R)-2-(2-azidoacetamido)cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (221), 10 mg, 0.010 mmol) and 2-(prop-2-yn-1-yl)phthalazin-1(2H)-one (5.7 mg, 0.031 mmol) to provide A-331 (2.5 mg, 0.003 mmol, 25%) (MW_{Calc}+H=970.44; MW_{Obs}=970.7).

A-332 was prepared in a similar manner to A-330 starting with (1R,2R)-3-(4-acetoxy-3,5-dimethylbenzamido)-1-((2R,3R,4S,6R)-4-acetoxy-6-(((1S,2R)-2-(2-azidoacetamido)cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (221, 10 mg, 0.010 mmol) and N-(prop-2-yn-1-yl)picolinamide (5.0 mg, 0.031 mmol) to provide A-332 (3.1 mg, 0.003 mmol, 31%) (MW_{Calc}+H=946.44; MW_{Obs}=946.7).

A-333 was prepared in a similar manner to A-330 starting with (1R,2R)-3-(4-acetoxy-3,5-dimethylbenzamido)-1-((2R,3R,4S,6R)-4-acetoxy-6-(((1S,2R)-2-(2-azidoacetamido)cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (221, 10 mg, 0.010 mmol) and ethynylbenzene (3.2 mg, 0.031 mmol) to provide A-333 (1.8 mg, 0.002 mmol, 18%) (MW_{Calc}+H=888.42; MW_{Obs}=888.6).

A-334 was prepared in a similar manner to A-330 starting with (1R,2R)-3-(4-acetoxy-3,5-dimethylbenzamido)-1-((2R,3R,4S,6R)-4-acetoxy-6-(((1S,2R)-2-(2-azidoacetamido)cyclohexyl)oxy)-3-(2-(4-cyclohexyl-1H-1,2,3-triazol-1-yl)acetamido)-6-(methoxycarbonyl)tetrahydro-2H-pyran-2-yl)propane-1,2-diyl diacetate (221, 10 mg, 0.010 mmol) and N-(prop-2-yn-1-yl)morpholine-4-carboxamide (5.2 mg, 0.031 mmol) to provide A-334 (4.4 mg, 0.005 mmol, 44%) (MW_{Calc}+H=54.46; MW_{Obs}=954.7).

### Preparation of A-335 and A-337

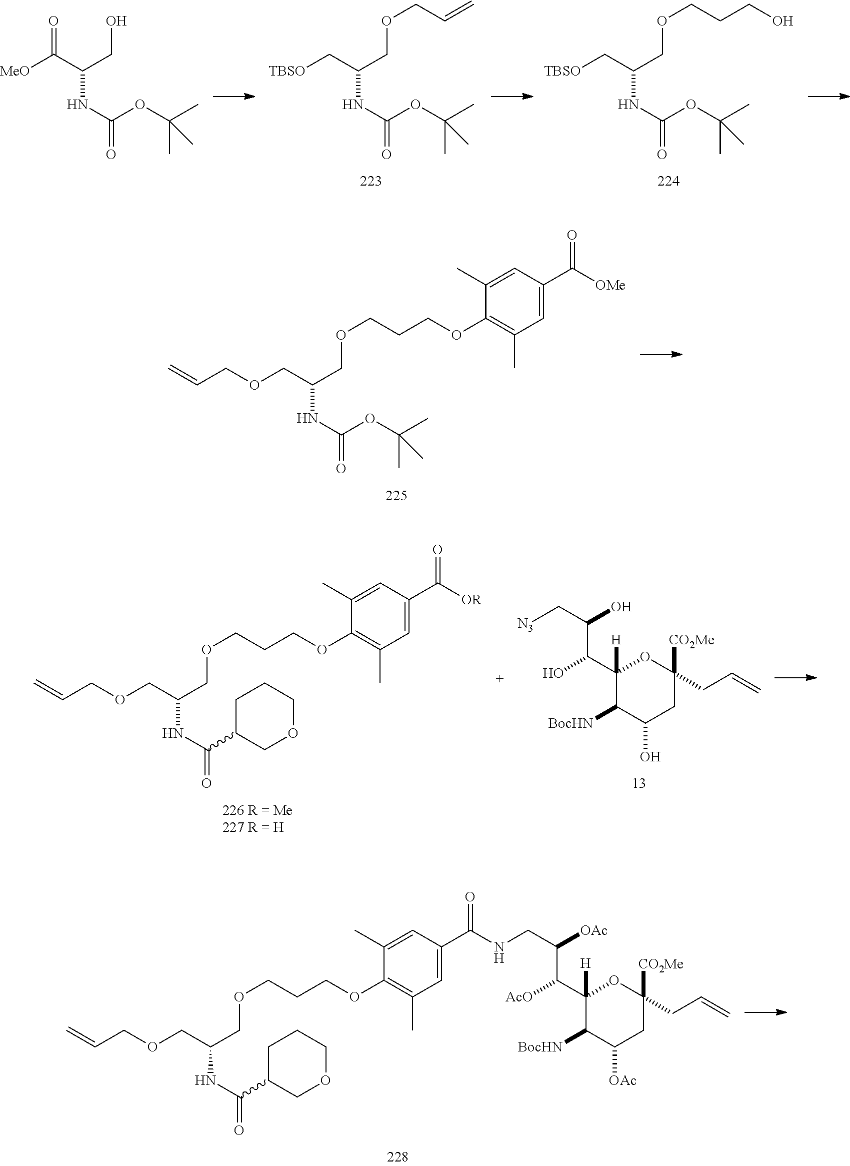

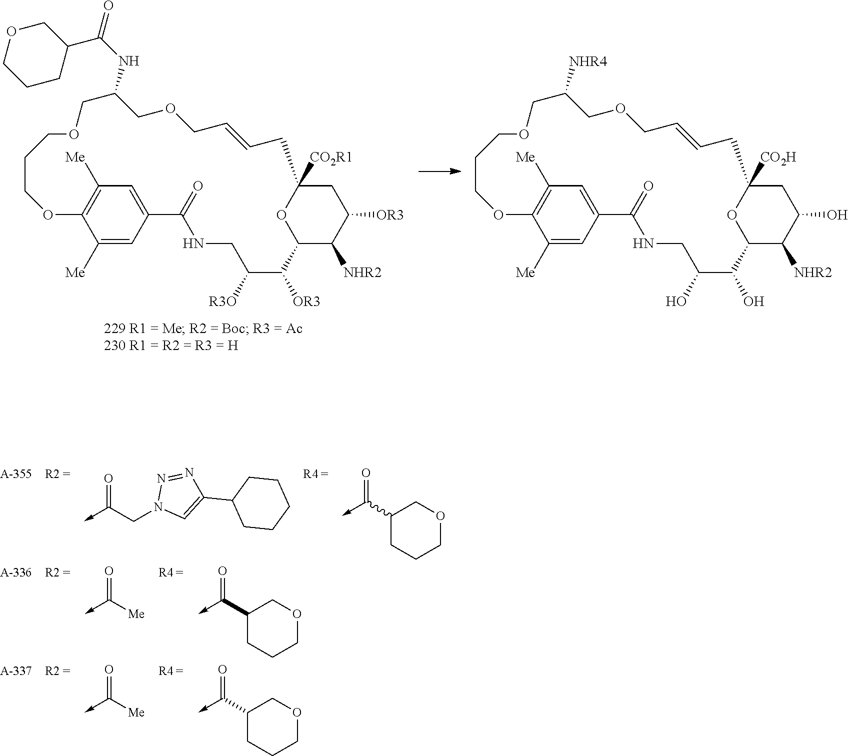

To a stirred solution of (S)-methyl 2-((tert-butoxycarbonyl)amino)-3-hydroxypropanoate (15 g, 68.42 mmol) in THF (450 mL) at room temperature under a N₂ atmosphere was added allyl methyl carbonate (10.80 mL, 95.788 mmol) and Pd(Ph₃P)₄ (1.581 g, 1.368 mmol). The reaction mixture was warmed at 60° C. and stirred for 5 h. The completed reaction was cooled to room temperature, diluted with EtOAc (500 mL), filtered through Celite (20 g), eluting with EtOAc (2×50 mL ea), and the combined filtrates were concentrated. The residue was dissolved in a 1:3 mixture of EtOAc in Heptane (100 mL) and filtered over a prepacked column of silica gel (200 g) eluting with 1:3 mixture of EtOAc in Heptane (1.0 L) collecting fractions (100 mL ea). The desired fractions were combined, concentrated, and the resultant residue was dried under vacuum. The dried residue was dissolved in THF (1.0 L) and added slowly dropwise over 1 h to a stirring solution of 1 M LiAlH4 (103 mL, 103 mmol) at 0° C. The total reaction was stirred for 2 h, after which time it was carefully quenched with sat. Na₂SO₄ (300 mL) and stirred for 2 h at room temperature. The suspension was filtered through silica gel (100 g) eluting with a 1:1 solution of EtOAc:heptane (500 mL), and the combined filtrates were concentrated to dry. The resultant residue was dissolved with stirring in DCM (180 mL) at room temperature followed by the addition of imidazole (13.97 g, 205.259 mmol) and TBS-CI (15.47 g, 102.63 mmol) at rt. The final reaction mixture was stirred for 4 h, after which time it was quenched with water (100 mL) and extracted with DCM (3×200 mL ea). The combined organic layers were dried over Na₂SO₄, filtered, and concentrated to dryness. The residue purified over a Biotage Ultra SNAP column (340 g) eluting with 10 CV of 0 to 50% EtOAc in heptane to provide compound 223 (17 g, 49.2 mmol, 72%) (MW_{Calc}+Na=368.23; MW_{Obs}=368.26) after combining the desired fractions, concentration, and drying under vacuum.

To a stirred solution of (S)-tert-butyl (1-(allyloxy)-3-((tert-butyldimethylsilyl)oxy)propan-2-yl)carbamate (223, 0.7 g, 2.026 mmol) in THF (8.40 mL) at 0° C. was added dropwise 9-BBN (12.15 mL, 6.077 mmol) over 5 min. The reaction mixture was slowly warmed to room temperature and stirred for 16 h. The reaction was cooled to 0° C., followed by the addition of H2O (10 mL), THF (5 mL), and then sodium perborate tetrahydrate (6.23 g, 40.515 mmol). The final mixture was warmed to room temperature, and then stirred for 16 h. The reaction was quenched with water (20 mL), and then extracted with EtOAc (3×10 mL ea). The combined organic layers were dried over Na₂SO₄, filtered, and concentrated to dryness. The residue purified over a Biotage Ultra SNAP column (25 g) eluting with 10 CV of 0 to 100% EtOAc in heptane to provide compound 224 (17 g, 49.2 mmol, 72%) after combining the desired fractions, concentration, and drying under vacuum.

To a stirred solution of (S)-tert-butyl (1-((tert-butyldimethylsilyl)oxy)-3-(3-hydroxypropoxy)propan-2-yl)carbamate (224, 0.65 g, 1.788 mmol) in THF (10 mL) at room temperature was added methyl 4-hydroxy-3,5-dimethylbenzoate (0.483 g, 2.682 mmol), and triphenylphosphine (0.703 g, 2.682 mmol), after which time the reaction was cooled to 0° C. DIAD (0.521 mL, 2.682 mmol) was slowly added, and then the mixture was warmed to room temperature and stirred for 3 h. The mixture was concentrated to ½ volume, and then purified directly over a Biotage Ultra SNAP column (25 g) eluting with 10 CV of 0 to 50% EtOAc in heptane to provide the fully protected intermediate after combining the desired fractions, concentration, and drying under vacuum. The resultant residue was dissolved with stirring in DCM (9.75 mL) at room temperature in a polypropylene vessel, followed by the addition of 2.3 M hydrogen fluoride in THF (2.353 mL, 5.364 mmol). The reaction was stirred for 16 h, after which time it was quenched with saturated NaHCO₃, and extracted with EtOAc (3×20 mL ea). The combined organic layers were concentrated, dried over Na₂SO₄, filtered, and concentrated to dryness. The residue purified over a Biotage Ultra SNAP column (25 g) eluting with 10 CV of 0 to 100% EtOAc in heptane to provide the intermediate alcohol after combining the desired fractions, concentration, and drying under vacuum. The alcohol was dissolve with stirring in THF (10 mL) at room temperature followed by the addition of Pd(Ph₃P)₄ (0.103 g, 0.089 mmol), and allyl methyl carbonate (0.406 ml, 3.576 mmol). The final reaction was warmed to 80° C. and stirred for 16 h. The completed reaction was cooled to room temperature, concentrated, and purified over a Biotage Ultra SNAP column (25 g) eluting with 10 CV of 5 to 20% EtOAc in heptane to provide compound 225 (0.6 g, 1.329 mmol, 74%) (MW_{Calc}+Na=474.26; MW_{Obs}=474.22) after combining the desired fractions, concentration, and drying under vacuum.

To a stirred solution of (S)-methyl 4-(3-(3-(allyloxy)-2-((tert-butoxycarbonyl)amino)propoxy)-propoxy)-3,5-dimethylbenzoate (225, 0.6 g, 1.329 mmol) in DCM (10 mL) at room temperature was slowly added TFA (1.024 ml, 13.287 mmol). The reaction mixture was stirred for 4 h, after which time it was concentrated, and azeotroped to dryness with toluene (2×10 mL ea). The residue was dissolved with stirring in acetonitrile (7.5 mL) at room temperature followed by the addition of tetrahydro-2H-pyran-3-carboxylic acid (0.259 g, 1.993 mmol), HOBT (0.203 g, 1.329 mmol), EDC (0.382 g, 1.993 mmol), and then triethylamine (0.556 ml, 3.986 mmol). The mixture was stirred for 15 h, after which time it was diluted with EtOAc (10 mL) and washed with water (5 mL) and brine (5 mL). The organic layer was dried over Na₂SO₄, filtered, and concentrated to dryness. The residue purified over a Biotage Ultra SNAP column (10 g) eluting with 10 CV of 0 to 40% EtOAc in heptane to provide compound 226 (0.42 g, 0.906 mmol, 68%) (MW_{Calc}+Na=486.26; MW_{Obs}=486.26) after combining the desired fractions, concentration, and drying under vacuum.

To a stirred solution of methyl 4-(3-((2S)-3-(allyloxy)-2-(tetrahydro-2H-pyran-3-carboxamido)-propoxy)propoxy)-3,5-dimethylbenzoate (226, 0.42 g, 0.906 mmol) in THF (9 mL) at room temperature was added MeOH (9 mL) and 1 N NaOH (9.06 mL, 9.06 mmol). The reaction was stirred for 16 h, after which time it was neutralized with 1 N HCl (9.0 mL), and brine (1 mL). The mixture was extracted with EtOAc (3×10 mL ea), dried over Na₂SO₄, filtered, and concentrated to dryness. The residue purified over a Biotage Ultra SNAP column (25 g) eluting with 10 CV of 40 to 100% EtOAc in heptane to provide compound 227 (0.315 g, 0.701 mmol, 77%) (MW_{Calc}+Na=472.24; MW_{Obs}=472.27) after combining the desired fractions, concentration, and drying under vacuum.

To a stirred solution of (2R,4S,5R,6R)-methyl 2-allyl-6-((1S,2S)-3-azido-1,2-dihydroxypropyl)-5-((tert-butoxycarbonyl)amino)-4-hydroxytetrahydro-2H-pyran-2-carboxylate (13, 0.1 g, 0.232 mmol) in THF (1.800 mL) and water (0.167 mL) at 0° C. was added 1N trimethylphosphine (0.697 mL, 0.697 mmol). The reaction mixture was allowed to warm to room temperature and stirred for 16 h. The completed reaction was concentrated, and azeotroped to dry with toluene (3×5 mL ea). The resultant crude amine was dissolved with stirring in MeCN (1.20 mL) at room temperature followed by the addition of 4-(3-((2S)-3-(allyloxy)-2-(tetrahydro-2H-pyran-3-carboxamido)propoxy)propoxy)-3,5-dimethylbenzoic acid (227, 0.157 g, 0.348 mmol), HOBT (0.036 g, 0.232 mmol), EDC (0.067 g, 0.348 mmol), and triethylamine (0.097 ml, 0.697 mmol). The reaction mixture was stirred for 6 h, after which time it was quenched with 0.5 N NaOH (2 mL) and extracted with EtOAc (4×5 mL ea). The combined organic layers were dried over Na₂SO₄, filtered, and concentrated to dryness. The residue was dissolved with stirring in DCM (1 mL) at room temperature followed by the addition of Et₃N (0.5 mL), DMAP (10 mg, 0.081 mmol), and Ac20 (0.219 mL, 2.323 mmol). The final reaction mixture was stirred for 16 h, after which time it was concentrated and directly purified over a Biotage Ultra SNAP column (10 g) eluting with 10 CV of 40 to 100% EtOAc in heptane to provide compound 228 (0.15 g, 0.156 mmol, 67%) (MW_{Calc}+H=962.48; MW_{Obs}=962.58) after combining the desired fractions, concentration, and drying under vacuum.

To a stirred solution of (1R,2R)-1-((2R,3R,4S,6R)-4-acetoxy-6-allyl-3-((tert-butoxycarbonyl)amino)-6-(methoxycarbonyl)tetrahydro-2H-pyran-2-yl)-3-(4-(3-((2S)-3-(allyloxy)-2-(tetrahydro-2H-pyran-3-carboxamido)propoxy)propoxy)-3,5-dimethylbenzamido)propane-1,2-diyl diacetate (228, 0.15 g, 0.156 mmol) in toluene (150 mL) at room temperature was added quinone (5.11 μL, 0.062 mmol) and Hoveyda-Grubbs Catalyst 2nd Generation (0.025 g, 0.039 mmol). The reaction mixture was warmed to 90° C. and stirred for 24 h. The completed reaction was cooled to room temperature, concentrated to dry and directly purified over a Biotage Ultra SNAP column (10 g) eluting with 10 CV of 20 to 100% EtOAc in heptane then 5 CV of 0 to 10% MeOH in EtOAc to provide compound 229 (0.11 g, 0.118 mmol, 76%) (MW_{Calc}+Na=956.45; MW_{Obs}=956.40) after combining the desired fractions, concentration, and drying under vacuum.

To a stirred solution of the protected macrocycle (229), 50 mg, 0.054 mmol) in THF (1.5 mL) and methanol (1.50 mL) at room temperature was added 1 N sodium hydroxide (1.60 mL, 1.60 mmol). The reaction mixture was stirred for 48 h, after which time it was neutralized with 1 N HCl (1.5 mL, 1.5 mmol) followed by the addition of brine (1 mL), and extraction with EtOAc (3×3 mL ea). The combined organic layers were washed with water (2 mL), brine (2 mL), dried over Na₂SO₄, filtered, and concentrated to dryness. The residue was dissolved with stirring in DCM (3 mL) at room temperature followed by the addition of TFA (250 μl, 3.245 mmol). The reaction mixture was stirred for 30 min, after which time it was concentrated, azeotroped to dry with toluene (3×5 mL ea) and dried under vacuum to provide crude 230 (MW_{Calc}+H=694.35; MW_{Obs}=9694.32), which was used in the next reaction without further purification.

To a stirred solution of 2-(4-cyclohexyl-1H-1,2,3-triazol-1-yl)acetic acid (12.06 mg, 0.058 mmol) in MeCN (0.2 mL) at room temperature was added HOBT (8.8 mg, 0.058 mmol), and EDC (11.1 mg, 0.058 mmol). The mixture was stirred for 1 h, after which time a mixture of the crude amine (230, 20 mg, 0.029 mmol) in MeCN (0.20 mL) was added followed by triethylamine (32 μL, 0.231 mmol). The reaction mixture was stirred for 4 h, after which time 1 N HCl (0.2 mL) and MeOH (1 mL) was added followed by purification directly via HPLC to provide compound A-335 (9 mg, 0.010 mmol, 35%) (MW_{Calc}+H=885.43; MW_{Obs}=885.45) after combining the desired fractions, concentration, and drying under vacuum.

To a stirred solution of the crude amine (230, 20 mg, 0.029 mmol) in THF (1.25 mL) was added sodium bicarbonate (4.5 mg, 0.054 mmol), and then acetic anhydride (5.0 μl, 0.054 mmol). The reaction was stirred for 15 min, after which time conc. HCL was slowly added to provide pH less than 2.0. The mixture was diluted with MeOH and purified directly via HPLC to provide compound A-336 (2 mg, 0.003 mmol, 5%) (MW_{Calc}+H=736.36; MW_{Obs}=736.34) and A-337 (4 mg, 0.005 mmol, 10%) (MW_{Calc}+H=736.36; MW_{Obs}=736.31) after combining the desired fractions, concentration, and drying under vacuum.

### Preparation of A-338 to A-342

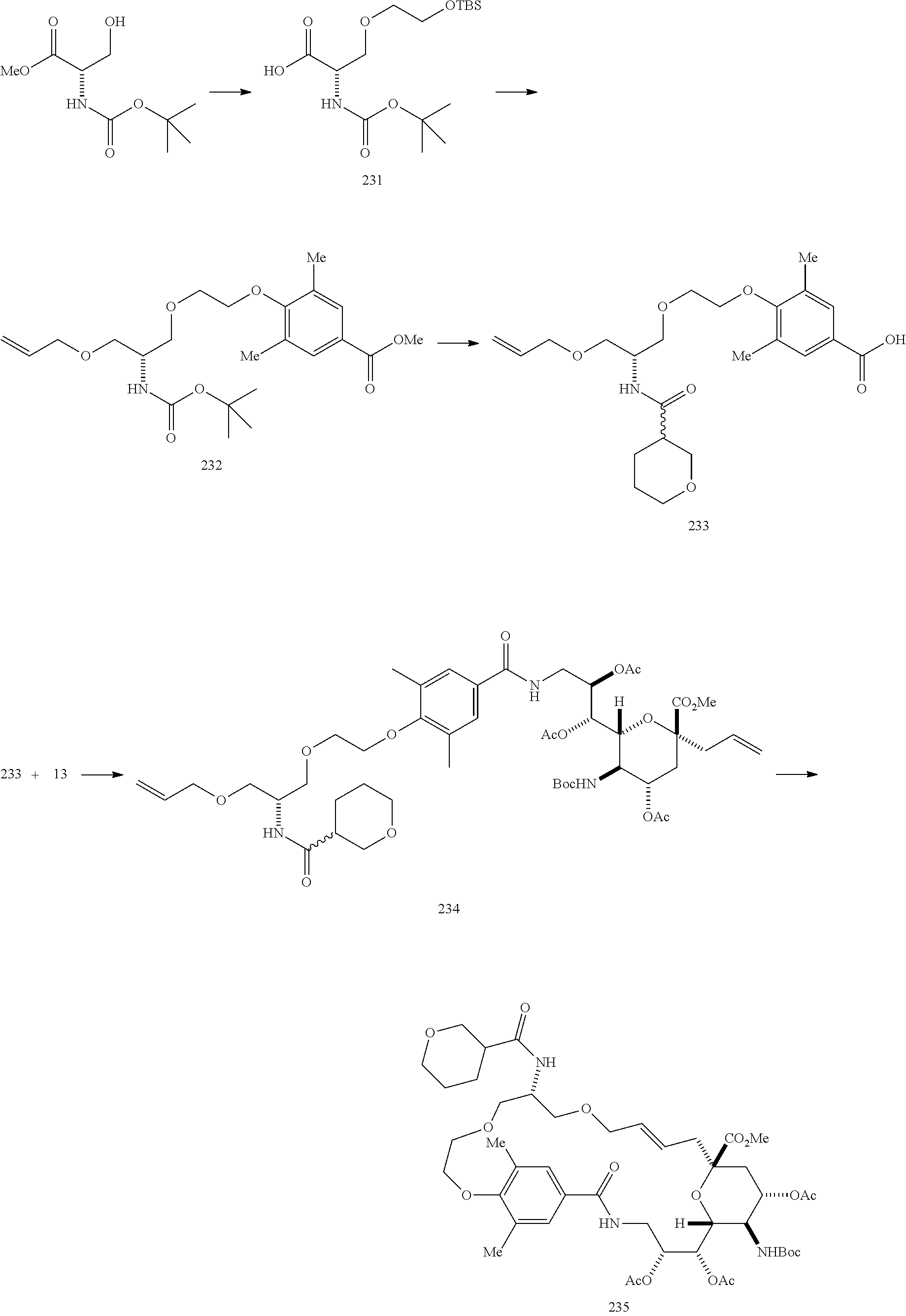

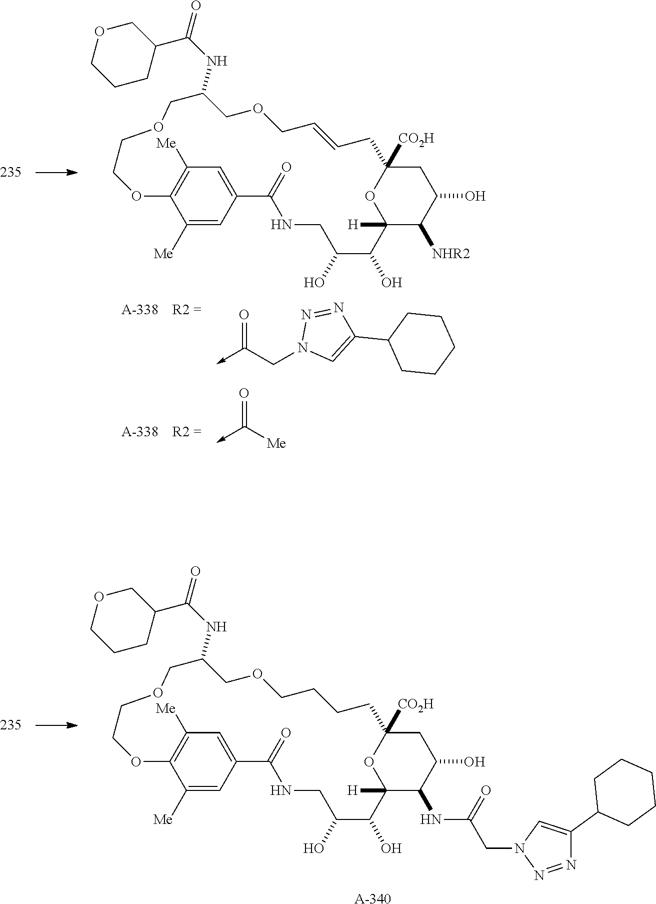

To a stirred solution of (S)-methyl 2-((tert-butoxycarbonyl)amino)-3-hydroxypropanoate (7 g, 31.93 mmol) in THF (210 mL) at room temperature under N₂ atmosphere was added allyl methyl carbonate (5.04 ml, 44.701 mmol) and Pd(Ph₃P)₄ (0.369 g, 0.319 mmol). The reaction mixture was warmed to 60° C. and stirred for 5 h. The completed reaction was cooled to room temperature, diluted with EtOAc (200 mL), and then filtered through a pad of Celite (20 g) eluting with EtOAc (3×50 mL ea). The combined filtrates were concentrated, and the residue was dissolved in a 1:3 mixture of EtOAc in heptane (100 mL) and filtered over a prepacked column of silica gel (200 g) eluting with 1:3 mixture of EtOAc in Heptane (1.0 L) collecting fractions (100 mL ea). The desired fractions were combined, concentrated, and the resultant residue was dried under vacuum to provide (S)-methyl 3-(allyloxy)-2-((tert-butoxycarbonyl)amino)propanoate (7.3 g, 28.2 mmol, 88%).

To a stirred solution of (S)-methyl 3-(allyloxy)-2-((tert-butoxycarbonyl)amino)propanoate (3.2 g, 12.341 mmol) in DCM (25.6 mL) and MeOH (25.6 mL) at −78° C. was subjected to bubbling ozone for 5-10 min. Upon completion dimethyl sulfide (6.35 mL, 86.387 mmol) was added dropwise, and the mixture was allowed to warm to room temperature, concentrated to dry. The crude aldehyde was dissolved in MeOH (25 mL) at 0° C. followed by the addition of sodium borohydride (0.700 g, 18.511 mmol). The reaction mixture was stirred for 1 h, after which time it was slowly quenched with sat NH₄Cl (25 mL), and the resultant slurry was extracted with EtOAc (3×25 mL ea). The combined organic layers were dried over Na₂SO₄, filtered and concentrated to dry. The crude alcohol was dissolved in DCM (25 mL) at room temperature followed by the addition of imidazole (4.20 g, 61.705 mmol) and TBS-CI (2.79 g, 18.511 mmol). The reaction was stirred for 2 h, after which time the mixture was concentrated, filtered through a short silica gel pad (25 g) eluting with EtOAc (3×25 mL ea, and the combined filtrates were concentrated to dry. The crude product was dissolved in THF (25 mL) and added dropwise to a stirring solution of LAH (0.937 g, 24.682 mmol) in THF (25 mL) at 0° C. The final reaction mixture was stirred at 0° C. for 1 h, after which time it was slowly quenched with sat. Na₂SO₄ (25 mL) followed by stirring at room temperature for 30 min. The suspension was filtered over Celite (25 g), eluting with EtOAc (3×25 mL ea), and the filtrates were concentrated to dry. The final residue was purified over a Biotage Ultra SNAP column (25 g) eluting with 10 CV of 20 to 100% EtOAc in heptane to provide compound 231 (1.00 g, 2.86 mmol, 23% yield) after combining the desired fractions, concentration, and drying under vacuum.

To a stirred solution of (R)-tert-butyl (1-(2-((tert-butyldimethylsilyl)oxy)ethoxy)-3-hydroxypropan-2-yl)carbamate (231, 1.00 g, 2.861 mmol) in THF (30.0 mL) at room temperature under N₂ atmosphere was added allyl methyl carbonate (0.452 ml, 4.005 mmol) and Pd(Ph₃P)₄ (0.033 g, 0.029 mmol). The reaction mixture was warmed to 60° C., and stirred for 5 h. The completed reaction was cooled to room temperature, diluted with EtOAc (20 mL), and then filtered through a pad of Celite (5 g) eluting with EtOAc (3×10 mL ea). The combined filtrates were concentrated, and the residue was dissolved in a 1:3 mixture of EtOAc in heptane (10 mL) and filtered over a prepacked column of silica gel (20 g) eluting with 1:3 mixture of EtOAc in Heptane (30 mL) collecting fractions (5 mL ea). The desired fractions were combined, concentrated, and the resultant residue was dried under vacuum to provide (R)-tert-butyl (2,2,3,3-tetramethyl-4,7,11-trioxa-3-silatetradec-13-en-9-yl)carbamate (0.8 g, 2.053 mmol, 72%).

To a stirred solution of (R)-tert-butyl (2,2,3,3-tetramethyl-4,7,11-trioxa-3-silatetradec-13-en-9-yl)carbamate (0.6 g, 1.54 mmol) in DCM (4.80 mL) at room temperature in a polypropylene vessel was added hydrogen fluoride triethylamine complex (0.377 mL, 2.31 mmol). The reaction mixture was stirred for 16 h, after which time it was quenched with saturated NaHCO₃, and extracted with DCM (3×10 mL ea). The combined organic layers were dried over Na₂SO₄, filtered and concentrated to dry. The residue was purified over a Biotage Ultra SNAP column (25 g) eluting with 10 CV of 20 to 100% EtOAc in heptane to provide (R)-tert-butyl (1-(allyloxy)-3-(2-hydroxyethoxy)propan-2-yl)carbamate (0.40 g, 1.453 mmol, 94%) after combining the desired fractions, concentration, and drying under vacuum.

To a stirred solution of (R)-tert-butyl (2,2,3,3-tetramethyl-4,7,11-trioxa-3-silatetradec-13-en-9-yl)carbamate (0.6 g, 1.54 mmol) in DCM (4.80 mL) at room temperature in a polypropylene vessel was added hydrogen fluoride triethylamine complex (0.377 mL, 2.31 mmol). The reaction mixture was stirred for 16 h, after which time it was quenched with saturated NaHCO₃, and extracted with DCM (3×10 mL ea). The combined organic layers were dried over Na₂SO₄, filtered and concentrated to dry. The residue was purified over a Biotage Ultra SNAP column (25 g) eluting with 10 CV of 20 to 100% EtOAc in heptane to provide (R)-tert-butyl (1-(allyloxy)-3-(2-hydroxyethoxy)propan-2-yl)carbamate (0.40 g, 1.453 mmol, 94%) after combining the desired fractions, concentration, and drying under vacuum.

To a stirred solution of (R)-methyl 4-(2-(3-(allyloxy)-2-((tert-butoxycarbonyl)amino)propoxy)ethoxy)-3,5-dimethylbenzoate (232, 0.53 g, 1.211 mmol) in DCM (5.30 mL) at room temperature was added TFA (0.93 mL, 12.114 mmol). The reaction mixture was stirred for 0.5 h, after which time the mixture was concentrated, and azeotroped to dry with toluene (2×20 mL ea). The crude amine was dissolved with stirring in DCM (5.30 mL) followed by the addition of were added triethylamine (0.844 ml, 6.057 mmol), tetrahydro-2H-pyran-3-carboxylic acid (0.236 g, 1.817 mmol), and HATU (0.599 g, 1.575 mmol). The reaction mixture was stirred for 2 h, after which time it was concentrated and purified directly over a Biotage Ultra SNAP column (25 g) eluting with 10 CV of 10 to 75% EtOAc in heptane to provide methyl 4-(2-((2R)-3-(allyloxy)-2-(tetrahydro-2H-pyran-3-carboxamido)propoxy)ethoxy)-3,5-dimethylbenzoate (0.51 g, 1.135 mmol, 94%) after combining the desired fractions, concentration, and drying under vacuum.

To a stirred solution of methyl 4-(2-((2R)-3-(allyloxy)-2-(tetrahydro-2H-pyran-3-carboxamido)-propoxy)ethoxy)-3,5-dimethylbenzoate (0.51 g, 1.135 mmol) in THF (11 mL) and MeOH (11 mL) at room temperature was added 1 N NaOH (11.35 ml, 11.35 mmol). The reaction was stirred for 16 h, after which time the mixture was neutralized with 1 N HCl (11.35 mL) plus brine (1 mL). The mixture was extracted with EtOAc (3×10 mL ea), and the combined organic layers were dried over Na₂SO₄, filtered and concentrated to dry. The residue was purified over a Biotage Ultra SNAP column (25 g) eluting with 10 CV of 20 to 100% EtOAc in heptane, and 5 CV of 0 to 10% MeOH in EtOAc to provide compound 233 (0.45 g, 1.033 mmol, 91% yield) (MW_{Calc}+Na=458.23; MW_{Obs}=458.18) after combining the desired fractions, concentration, and drying under vacuum.

To a stirred solution of 4-(2-((2R)-3-(allyloxy)-2-(tetrahydro-2H-pyran-3-carboxamido)-propoxy)ethoxy)-3,5-dimethylbenzoic acid (233, 0.258 g, 0.593 mmol) and (2R,4S,5R,6R)-methyl 2-allyl-6-((1R,2R)-3-amino-1,2-dihydroxypropyl)-5-((tert-butoxycarbonyl)amino)-4-hydroxytetrahydro-2H-pyran-2-carboxylate (13, 0.160 g, 0.395 mmol) in MeCN (3 mL) at room temperature was added triethylamine (0.165 mL, 1.185 mmol), HOBT (0.030 g, 0.198 mmol), and EDC (0.106 g, 0.553 mmol). The reaction mixture was stirred for 24 h, after which time it was diluted with EtOAc (5 mL), and the organic layer was washed with water (3 mL), and brine (3 mL). The resultant organic layer was dried over Na₂SO₄, filtered and concentrated to dry. The residue was dissolved with stirring in pyridine (1.60 mL) at room temperature followed by the addition of Ac₂O (0.745 ml, 7.90 mmol) and DMAP (4.83 mg, 0.04 mmol). The final reaction mixture was stirred for 16 h, after which time the mixture was quenched with aqueous NH₄Cl (3 mL) and extracted with EtOAc (3×5 mL ea). The combined organic layers were dried over Na₂SO₄, filtered and concentrated to dry. The residue was purified over a Biotage Ultra SNAP column (10 g) eluting with 10 CV of 30 to 100% EtOAc in heptane to provide compound 234 (0.25 g, 0.264 mmol, 67% yield) (MW_{Calc}+H=948.46; MW_{Obs}=948.45) after combining the desired fractions, concentration, and drying under vacuum.

To a stirred solution of (1R,2R)-1-((2R,3R,4S,6R)-4-acetoxy-6-allyl-3-((tert-butoxycarbonyl)amino)-6-(methoxycarbonyl)tetrahydro-2H-pyran-2-yl)-3-(4-(2-((2R)-3-(allyloxy)-2-(tetrahydro-2H-pyran-3-carboxamido)propoxy)ethoxy)-3,5-dimethylbenzamido)propane-1,2-diyl diacetate (234, 130 mg, 0.137 mmol) in toluene (130 mL) at room temperature was added quinone (4.50 μl, 0.055 mmol) and Hoveyda-Grubbs Catalyst 2nd Generation (21.55 mg, 0.034 mmol). The reaction mixture was warmed to 80° C. and stirred for 3 h. The completed reaction was cooled to room temperature, concentrated to dry and directly purified on four preparative TLC plates eluting with 10% MeOH in EtOAc to provide compound 235 (75 mg, 0.082 mmol, 60%) (MW_{Calc}+H=920.43; MW_{Obs}=920.35) after combining the desired fractions, eluting the product off the silica gel with 10% MeOH in EtOAc (4×5 mL ea) via filtration, concentration of the filtrate, and drying under vacuum.

To a stirred solution of the protected macrocycle (235, 55 mg, 0.06 mmol) in THF (0.6 mL) and methanol (0.6 mL) at room temperature was added 1 N NaOH (1.196 mL, 1.196 mmol). The reaction mixture was stirred at for 30 h, after which time 1N HCl (2 mL) and brine (1 mL) were added followed by extraction with EtOAc (3×3 mL ea). The combined organic layers were dried over Na₂SO₄, filtered and concentrated to dry. The residue was dissolved with stirring in DCM (1 mL) at room temperature followed by the addition of TFA (46.1 μl, 0.598 mmol). The reaction mixture was stirred for 12 min, after which time it was concentrated, and azeotroped to dry with toluene (3×5 mL ea) to provide the crude amine intermediate.

To a stirred solution of 2-(4-cyclohexyl-1H-1,2,3-triazol-1-yl)acetic acid (12.51 mg, 0.06 mmol) in MeCN (0.55 mL) at room temperature was added HOBT (16.48 mg, 0.108 mmol) and EDC (20.63 mg, 0.108 mmol). The mixture was stirred for 2 h, after which time a mixture of the crude amine (ca 20 mg) from above in MeCN (0.5 mL) with triethylamine (41.7 μL, 0.299 mmol) was added. The final reaction mixture was stirred for 3 h, after which time it was quenched with 0.1 N HCl (1 mL) and extracted with EtOAc (3×3 mL ea). The combined organic layers were dried over Na₂SO₄, filtered, concentrated, and purified by HPLC to provide A-338 (9 mg, 0.010 mmol, 17%) (MW_{Calc}+H=871.44; MW_{Obs}=871.40) after combining the desired fractions, concentration, and drying under vacuum.

To a stirred solution of the crude amine (ca 20 mg) in THF (0.5 mL) was added 10% sodium bicarbonate in water (247 mg, 0.294 mmol) and then acetic anhydride (22.2 μL, 0.235 mmol). The reaction was stirred for 15 min, after which time conc HCl (14.07 μl, 0.167 mmol) was added to provide pH<2. The mixture was dilute in MeOH (1.5 mL) and purified directly by HPLC to provide A-339 (12 mg, 0.017 mmol, 57%) (MW_{Calc}+H=722.35; MW_{Obs}=722.28) after combining the desired fractions, concentration, and drying under vacuum.

A-341 was prepared in a similar fashion to A-338 starting using (R)-methyl 2-((tert-butoxycarbonyl)amino)-3-hydroxypropanoate instead of (S)-methyl 2-((tert-butoxycarbonyl)amino)-3-hydroxypropanoate. The same reaction sequence provided A-341 (4 mg, 0.005 mmol) (MW_{Calc}+H=871.44; MW_{Obs}=871.37).

To a stirred solution of the protected macrocycle compound 235 (6 mg, 7.694 μmol) in MeOH (2 mL) at room temperature was purged with N₂ atmosphere (3×) followed by the addition of 10% Pd—C (0.082 mg, 0.769 μmol). The mixture was purged with H₂ (3×) and stirred under a H₂ atmosphere under balloon pressure for 2 h. The completed reaction was purged with N₂ atmosphere (3×) followed by filtering over a pad Celite (3 g) eluting with 10% MeOH in EtOAc (3×5 mL ea). The combined filtrates were concentrated, dissolved with stirring in DCM (1 mL) at room temperature followed by the addition of TFA (5.93 μL, 0.077 mmol). The reaction mixture was stirred for 12 min, after which time it was concentrated, and azeotroped to dry with toluene (3×5 mL ea) to provide the crude amine intermediate.

To a stirred solution of 2-(4-cyclohexyl-1H-1,2,3-triazol-1-yl)acetic acid (2.4 mg, 0.012 mmol) in MeCN (1 mL) at room temperature was added HOBT (1.6 mg, 0.011 mmol) and EDC (2.1 mg, 0.011 mmol). The mixture was stirred for 2 h, after which time a mixture of the crude amine from above in MeCN (0.5 mL) with triethylamine (5.4 μL, 0.038 mmol) was added. The final reaction mixture was stirred for 3 h, after which time it was quenched with 0.1 N HCl (0.3 mL) and extracted with EtOAc (3×3 mL ea). The combined organic layers were dried over Na₂SO₄, filtered, concentrated, and purified by HPLC to provide A-340 (4 mg, 0.005 mmol, 60%) (MW_{Calc}+H=873.46; MW_{Obs}=846.33) after combining the desired fractions, concentration, and drying under vacuum.

A-342 was prepared in a similar fashion to A-340 and A-339 starting using (R)-methyl 2-((tert-butoxycarbonyl)amino)-3-hydroxypropanoate instead of (S)-methyl 2-((tert-butoxycarbonyl)amino)-3-hydroxypropanoate. The same reaction sequences provided A-342 (6 mg, 0.008 mmol) (MW_{Calc}+H=724.46; MW_{Obs}=724.26).

### Preparation of A-343

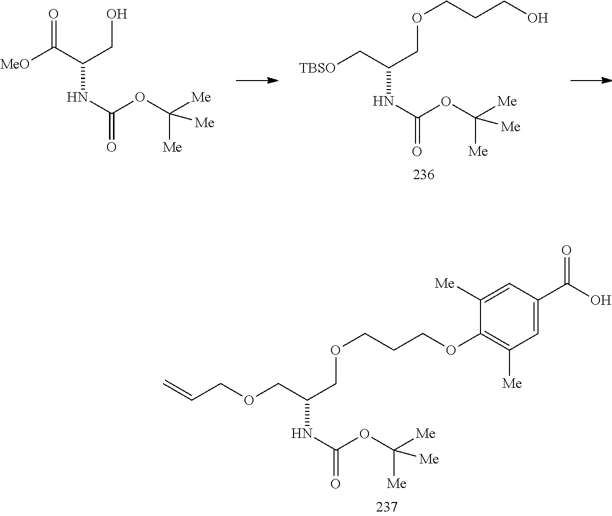 [SMILES: COC(=O)[C@H](CO)NC(=O)OC(C)(C)C ; CC(C)(C)OC(=O)N[C@@H](COCCCO)CO[Si](C)(C)C(C)(C)C ; C=CCOC[C@H](COCCCOc1c(C)cc(C(=O)O)cc1C)NC(=O)OC(C)(C)C]

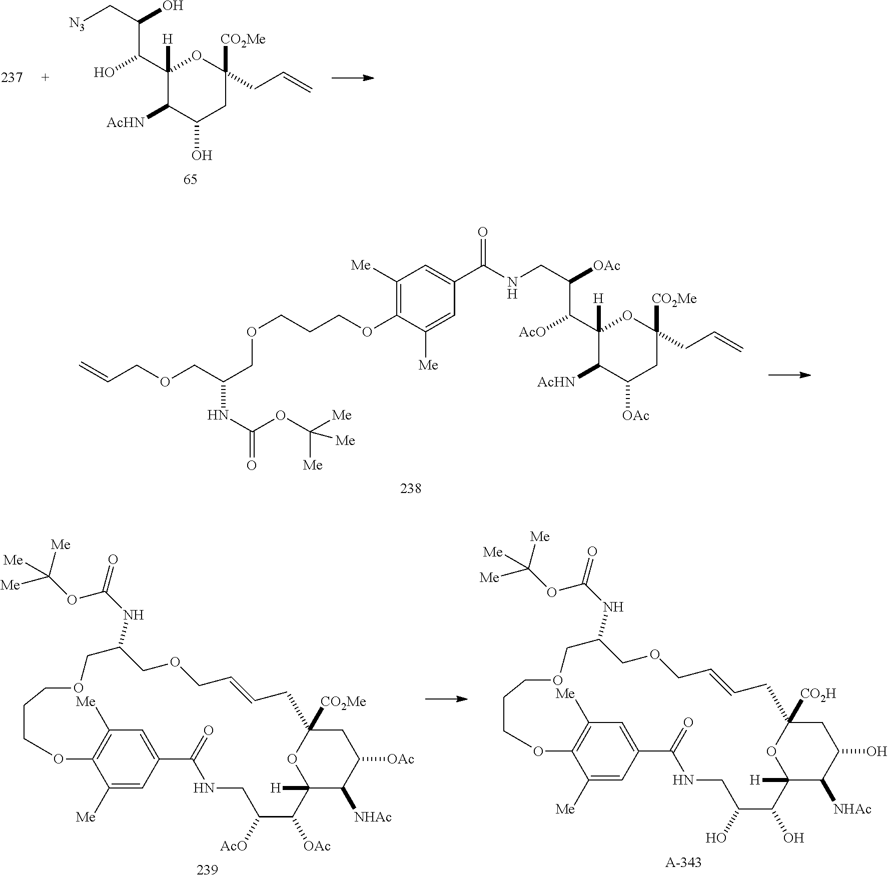

To a stirred solution of (S)-methyl 2-((tert-butoxycarbonyl)amino)-3-hydroxypropanoate (15 g, 68.42 mmol) in THF (450 ml) was added allyl methyl carbonate (10.80 mL, 95.788 mmol) and Pd(Ph₃P)₄ (1.581 g, 1.368 mmol). The reaction mixture was warmed to 60° C. and stirred for 5 hours. The completed intermediate was diluted with EtOAc (200 mL), filtered through a plug of Celite, the filter pad washed with EtOAc (2×50 mL ea), and the combined filtrates were concentrated to provide the crude ester.

To a stirred solution of 1 M lithium aluminum hydride in THF (103 mL, 103.0 mmol) in THF (900 mL) at 0° C. was added the above crude ester in THF (100 mL) over a 30-minute period. The reaction mixture was stirred for 2 h, after which time it was slowly quench with the dropwise addition of sat. Na₂SO₄ in water. The quench suspension was stirred for an additional 2 h at room temperature, followed by filtering through a plug of silica gel (50 g) eluting with ethyl acetate (2×100 mL ea). The combined filtrate was concentrated, azeotroped to dry with toluene (2×100 mL ea) and dried under high vacuum. The residue was dissolved in DCM (180 mL) at room temperature followed by the addition of imidazole (13.97 g, 205.26 mmol) and tert-butyldimethylsilyl chloride (15.47 g, 102.63 mmol). The final mixture was stirred for 4 hours, after which time it was quenched with water (50 mL), the layers separated, and the aqueous layer was extracted with dichloromethane (2×30 mL ea). The combined organic layers were dried over Mg₂SO₄, filtered, and concentrated. The residue was purified over a plug of silica gel eluting with 4:1 heptane:ethyl acetate (5 CV) to provide (S)-tert-butyl (1-(allyloxy)-3-((tert-butyldimethylsilyl)oxy)propan-2-yl)carbamate (17.0 g. 49.2 mmol, 72%) (MW_{Calc}+Na=368.23; MW_{Obs}=368.26) after concentration of the desired fractions and drying under vacuum.

To a stirred solution of(S)-tert-butyl (1-(allyloxy)-3-((tert-butyldimethylsilyl)oxy)propan-2-yl)carbamate (6.00 g, 117.36 mmol) in THF (72 mL) at 0° C. was added 0.5 M 9-BBN in THF (87 mL, 43.41 mmol) dropwise over a 20-minute period. The reaction mixture was slowly warmed to room temperature and stirred for an additional 16 h. The completed intermediate was cooled to 0° C., diluted with water (100 mL) and THF (50 mL), followed by a piecemeal addition of sodium perborate tetrahydrate (53.40 g, 347.27 mmol). The final reaction was warmed to room temperature and stirred for an additional 16 h. The completed reaction was diluted with water (200 mL), extracted with ethyl acetate (2×300 mL ea), and the combined organic layers were dried over Na₂SO₄, filtered and concentrated to dry. The residue was purified over a Biotage Ultra SNAP column (100 g) eluting with 10 CV of 10 to 100% EtOAc in heptane to provide compound 236 (4.1 mg, 11.28 mmol, 65%) after concentration of the desired fractions and drying under vacuum.

To a stirred solution of (S)-tert-butyl (1-((tert-butyldimethylsilyl)oxy)-3-(3-hydroxypropoxy)propan-2-yl)carbamate (236, 4.00 g, 11.002 mmol) in THF (60 mL) was added methyl 4-hydroxy-3,5-dimethylbenzoate (2.97 g, 16.503 mmol) followed by triphenylphosphine (4.33 g, 16.50 mmol). The mixture was cooled to 0° C., followed by the addition of DIAD (3.21 ml, 16.50 mmol). The reaction mixture was warmed to room temperature and stirred for an additional 3 hours. The completed reaction was concentrated, and the residue was purified directly over a Biotage Ultra SNAP column (50 g) eluting with 10 CV of 0 to 50% EtOAc in heptane to provide the crude phenolic ether after concentrating the desired fractions to dryness. The residue was dissolved with stirring in DCM (60.0 ml) at room temperature followed by a dropwise additional of Et₃N 3HF (5.36 ml, 33.01 mmol) over a 5-minute period. The mixture was stirred for 16 hours, after which time it was slowly quenched with sat. sodium bicarbonate (80 mL), and then extracted with DCM (3×100 mL ea). The combined organic layers were dried over MgSO₄, filtered and concentrated to dry. The residue purified over a Biotage Ultra SNAP column (50 g) eluting with 10 CV of 0 to 100% EtOAc in heptane to provide the crude alcohol after concentrating the desired fractions to dryness. The residue was dissolved with stirring in THF (100 mL) at room temperature followed by Pd(Ph₃P)₄ (0.636 g, 0.55 mmol) and allyl methyl carbonate (2.50 mL, 22.00 mmol). The final reaction mixture was warmed to 80° C., stirred for 16 h. The completed reaction was concentrated to dryness followed by purifying directly of a Biotage Ultra SNAP column (50 g) eluting with 10 CV of 5 to 20% EtOAc in heptane to provide (S)-methyl 4-(3-(3-(allyloxy)-2-((tert-butoxycarbonyl)amino)propoxy)propoxy)-3,5-dimethylbenzoate (3.0 g, 6.64 mmol, 60% yield) after concentration of the desired fractions and drying under vacuum.

To a stirred solution of (S)-methyl 4-(3-(3-(allyloxy)-2-((tert-butoxycarbonyl)amino)propoxy)-propoxy)-3,5-dimethylbenzoate (3 g, 6.644 mmol) in THF (45.0 mL) and MeOH (45.0 mL) at room temperature was added 1.0 N NaOH (33.2 mL, 33.2 mmol). The reaction mixture was stirred for 16 h, after which time it was acidified with 1N HCl (40 mL) and diluted with EtOAc (80 mL). The layers were separated, and the aqueous layer was extracted with EtOAc (3×25 mL ea). The combined organic layers were dried over Na₂SO₄, filtered and concentrated to dry. The residue was purified over a Biotage Ultra SNAP column (25 g) eluting with 10 CV of 20 to 80% EtOAc in heptane to provide 237 (2.7 g, 6.17 mmol, 93%). (MW_{Calc}+Na=460.24; MW_{Obs}=460.26).

To a stirred solution of (2R,4S,5R,6R)-methyl 5-acetamido-2-allyl-6-((1R,2R)-3-azido-1,2-dihydroxypropyl)-4-hydroxytetrahydro-2H-pyran-2-carboxylate (65, 0.8 g, 2.148 mmol) in THF (16.00 mL) and water (1.548 mL) at 0° C. was added 1N trimethylphosphine (0.570 ml, 6.445 mmol). The reaction mixture was warmed to room temperature, stirred for 16 h, then concentrated, and azeotroped to dry with toluene (3×30 mL ea). The resultant residue was added to a stirring solution (S)-4-(3-(3-(allyloxy)-2-((tert-butoxycarbonyl)amino)propoxy)propoxy)-3,5-dimethylbenzoic acid (237, 1.410 g, 3.223 mmol) in MeCN (9.60 mL) at room temperature followed by the addition of triethylamine (2.99 mL, 21.484 mmol), HOBT (0.329 g, 2.148 mmol), and EDC (0.618 g, 3.223 mmol). The resultant reaction mixture was stirred for 6 h, after which time it was concentrated. The residue was dissolved with stirring in DCM (10 mL) at room temperature followed by the addition of triethylamine (2.99 mL, 21.484 mmol), DMAP (0.026 g, 0.215 mmol) and Ac₂O (1.419 ml, 15.039 mmol). The final reaction mixture was stirred for 16 h, after which time it was quenched with sat NaHCO₃ (20 mL) and 0.2 N NaOH (20 mL). The mixture was extracted with EtOAc (3×60 mL ea), and the combined organic layers were dried over Na₂SO₄, filtered and concentrated to dry. The residue was purified over a Biotage Ultra SNAP column (50 g) eluting with 10 CV of 30 to 100% EtOAc in heptane to provide compound 238 (86 mg, 0.096 mmol, 4.5%) (MW_{Calc}+H=892.44; MW_{Obs}=892.52) along with peracetylated compound 65 (1R,2R)-1-((2R,3R,4S,6R)-3-acetamido-4-acetoxy-6-allyl-6-(methoxycarbonyl)tetrahydro-2H-pyran-2-yl)-3-azidopropane-1,2-diyl diacetate (0.22 g, 0.441 mmol, 21%) after combining the desired fractions, concentration, and drying under vacuum.

To a stirred solution of (1R,2R)-1-((2R,3R,4S,6R)-3-acetamido-4-acetoxy-6-allyl-6-(methoxycarbonyl)-tetrahydro-2H-pyran-2-yl)-3-(4-(3-((S)-3-(allyloxy)-2-((tert-butoxycarbonyl)amino)propoxy)propoxy)-3,5-dimethylbenzamido)propane-1,2-diyl diacetate (238, 86 mg, 0.096 mmol) in toluene (86 mL) at room temperature was added quinone (3.16 μL, 0.039 mmol) and Hoveyda-Grubbs Catalyst 2nd Generation (15.15 mg, 0.024 mmol). The reaction mixture was warmed to 100° C., stirred for of 3 h followed by the addition of DMSO (0.1 mL), and stirred for an additional 3 h at 100° C. The mixture was cooled to room temperature, concentrated to an oil, and purified over Biotage Ultra SNAP column (25 g) eluting with 10 CV gradient of 20 to 100% EtOAc in heptane, and a 5 CV gradient of 0 to 10% MeOH in EtOAc to provide compound 239 (70 mg, 0.081 mmol, 84%) (MW_{Calc}+Na=886.41; MW_{Obs}=886.57).

To a stirred solution of the fully protected macrocycle (237, 20 mg, 0.023 mmol) in MeOH (0.40 mL) and THF (0.40 mL) at room temperature was added 1 N NaOH (463 μl, 0.463 mmol). The reaction mixture was stirred for 72 h, after which time it was acidified to pH 4 with conc. HCl and purified by HPLC to provide A-343 (6 mg, 0.008 mmol, 36%) (MW_{Calc}+H=724.46; MW_{Obs}=724.49) after combining the desired fractions, concentration, and drying under vacuum.

### Preparation of A-344

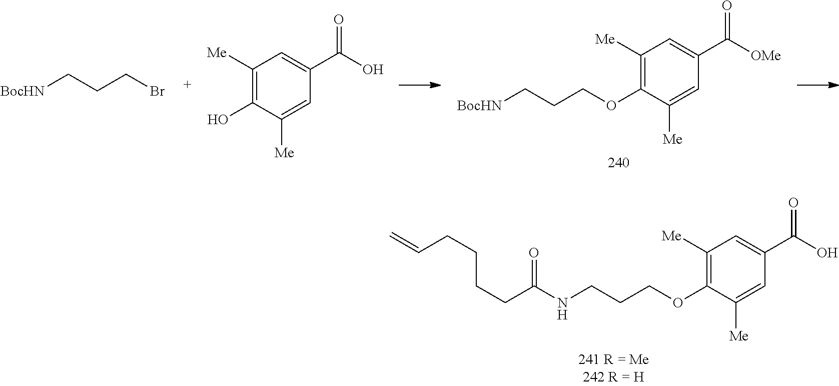 [SMILES: CC(C)(C)OC(=O)NCCCBr ; Cc1cc(C(=O)O)cc(C)c1O ; COC(=O)c1cc(C)c(OCCCNC(=O)OC(C)(C)C)c(C)c1 ; C=CCCCCC(=O)NCCCOc1c(C)cc(C(=O)O)cc1C]

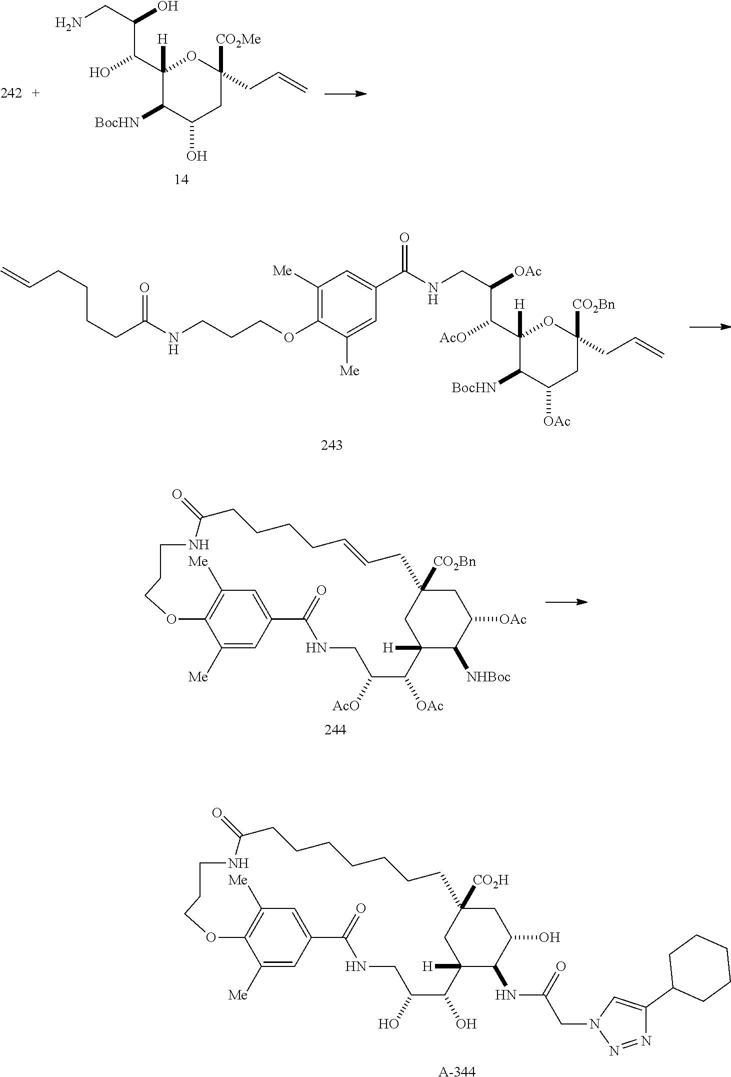

To a stirred solution of 4-hydroxy-3,5-dimethylbenzoic acid (0.5 g, 3.009 mmol) in DMF (10.0 mL) at room temperature was added potassium carbonate (1.248 g, 9.027 mmol) followed by tert-butyl (3-bromopropyl)carbamate (2.149 g, 9.027 mmol). The reaction was warmed to 110° C. and stirred for 2 h. The reaction was cooled to room temperature, added additional potassium carbonate (1.248 g, 9.027 mmol) and tert-butyl (3-bromopropyl)carbamate (2.149 g, 9.027 mmol), followed by warming to 100° C. and stirred for 2 h. The resultant mixture was cooled to room temperature then diluted with water (20 mL) and extracted with EtOAc (4×20 mL ea). The combined organic layers were washed with water (2×20 mL ea) and brine (20 mL). The combined organic layers were dried over Na₂SO₄, filtered and concentrated to dry. The residue was purified a Biotage Ultra SNAP column (50 g) eluting with 10 CV gradient of 0 to 25% EtOAc in heptane to provide 3-((tert-butoxycarbonyl)amino)propyl 4-(3-((tert-butoxycarbonyl)amino)propoxy)-3,5-dimethylbenzoate (540 mg, 1.124 mmol, 37%) as an oil after combining the desired fractions, concentration, and drying under vacuum. The oil was dissolved with stirring in MeOH (5 mL) and THF (5 mL) at room temperature followed by the addition of 1N NaOH (10 mL). The reaction mixture was stirred for 5 h, after which time it was acidified to pH<2 with the addition of 1 N HCl. The resulting mixture was extracted with EtOAc (3×20 mL ea), and the combined organic layers dried over Na₂SO₄, filtered and concentrated to dry. The residue was dissolved with stirring in toluene (5.0 mL) and methanol (0.5 mL) at room temperature followed by the addition of 2.0M TMS-diazomethane (1.504 mL, 3.009 mmol). The final reaction mixture was stirred for 20 min, after which time it was concentrated and purified directly over Biotage Ultra SNAP column (10 g) eluting with 10 CV gradient of 0 to 50% EtOAc in heptane to provide compound 240 (316 mg, 0.937 mmol, 31%) (MW_{Calc}+Na=360.19; MW_{Obs}=360.26) as a crystalline solid after combining the desired fractions, concentration, and drying under vacuum.

To a stirred solution of methyl 4-(3-((tert-butoxycarbonyl)amino)propoxy)-3,5-dimethylbenzoate (240, 162 mg, 0.48 mmol) in DCM (1.134 mL) at room temperature was added TFA (1.134 mL, 14.719 mmol). The reaction mixture was stirred for 10 min, after which time it was concentrated and azeotroped to dry with toluene (2×10 mL ea). The crude residue was dissolved with stirring in MeCN (1.0 mL) followed by the addition of 6-heptenoic acid (98 μL, 0.72 mmol), Et₃N (268 μL, 1.921 mmol), and HATU (319 mg, 0.84 mmol). The reaction mixture was stirred for 45 min, after which time water (2 mL) was added, and the resulting mixture was extracted with EtOAc (2×5 mL ea). The combined organic layers were washed with water (2 mL) and brine (2 mL). The organic layer was dried over Na₂SO₄, filtered and concentrated to dry. The residue was purified over Biotage Ultra SNAP column (10 g) eluting with 10 CV gradient of 4 to 50% EtOAc in heptane to provide compound 241 (165 mg, 0.475 mmol, 99%) (MW_{Calc}+H=370.21; MW_{Obs}=370.33) as a white crystalline solid after combining the desired fractions, concentration, and drying under vacuum.

To a stirred solution of methyl 4-(3-(hept-6-enamido)propoxy)-3,5-dimethylbenzoate (241, 160 mg, 0.46 mmol) in methanol (2.40 mL) at room temperature was added 1 N sodium hydroxide (1.381 mL, 1.381 mmol). The reaction mixture was stirred for 4 h, after which time it was acidified to pH 2, and then extracted with EtOAc (5×2 mL ea). The combined organic layers were washed with brine (2 mL), and the combined organic layers were dried over Na₂SO₄, filtered and concentrated to dry to provide the crude compound 242) (145 mg, 0.435 mmol, 94%) (MW_{Calc}+H=334.19; MW_{Obs}=334.32) as a white solid.

To a stirred solution of (2R,4S,5R,6R)-methyl 2-allyl-6-((1R,2R)-3-amino-1,2-dihydroxypropyl)-5-((tert-butoxycarbonyl)amino)-4-hydroxytetrahydro-2H-pyran-2-carboxylate (14, 80 mg, 0.198 mmol) and 4-(3-(hept-6-enamido)propoxy)-3,5-dimethylbenzoic acid (242, 82 mg, 0.247 mmol) in MeCN (1 mL) at room temperature and triethylamine (83 μL, 0.593 mmol), HOBT (15.2 mg, 0.099 mmol), and EDC (56.9 mg, 0.297 mmol). The reaction mixture was stirred for 6 h, after which time it was diluted with EtOAc (5 mL) and washed with water (1 mL) and brine (1 mL). The organic layer was dried over Na₂SO₄, filtered, concentrated, and purified over Biotage Ultra SNAP column (4 g) eluting with 10 CV gradient of 0 to 100% EtOAc in heptane to provide the desired amide (112 mg) as a transparent film. The resulting amide was dissolved in methanol with stirring (1.0 mL) at room temperature followed by 1 N sodium hydroxide (0.528 mL, 0.528 mmol). The reaction mixture was stirred for 24 h, after which time it was acidified with 1N HCL (0.7 mL) and extracted extensively with EtOAc (5×2 mL ea). The combined organic layers were washed with brine (2 mL), and dried over Na₂SO₄, filtered and concentrated to dry to provide the desired crude acid.

To a stirred solution of crude (2R,4S,5R,6R)-2-allyl-5-((tert-butoxycarbonyl)amino)-6-((1R,2R)-3-(4-(3-(hept-6-enamido)propoxy)-3,5-dimethylbenzamido)-1,2-dihydroxypropyl)-4-hydroxytetrahydro-2H-pyran-2-carboxylic acid in DMF (1.2 mL) at room temperature was added potassium carbonate (82 mg, 0.593 mmol) and benzyl bromide (70.6 μL, 0.593 mmol). The reaction mixture was stirred for 2 h, after which time water (5 mL) was added, and the mixture was extracted with EtOAc (2×5 mL). The combined organic layers were washed with water (2 mL) and brine (2 mL). The organic layer was dried over Na₂SO₄, filtered and concentrated to dry to provide the desired crude benzyl ester as an oil. The crude residue was dissolved with stirring in pyridine (0.3 mL) at room temperature followed by the addition of acetic anhydride (0.3 mL, 3.18 mmol). The final reaction mixture was stirred for 14 h, after which time EtOAc (10 mL) was added, and the resulting mixture was washed with NH₄Cl (3 mL), 1N HCl (3 mL), and brine (3 mL). The organic layer was dried over Na₂SO₄, filtered, concentrated, and purified over a Biotage Ultra SNAP column (4 g) eluting with 10 CV gradient of 0 to 100% EtOAc in heptane to provide compound 243 (79 mg, 0.086 mmol, 43%) (MW_{Calc}+H=922.46; MW_{Obs}=922.56) as a film after combining the desired fractions, concentration, and drying under vacuum.

To a stirred solution of (1R,2R)-1-((2R,3R,4S,6R)-4-acetoxy-6-allyl-6-((benzyloxy)carbonyl)-3-((tert-butoxycarbonyl)amino)tetrahydro-2H-pyran-2-yl)-3-(4-(3-(hept-6-enamido)propoxy)-3,5-dimethylbenzamido)propane-1,2-diyl diacetate (243, 40 mg, 0.043 mmol) in toluene (20 mL) at room temperature was added Hoveyda-Grubbs Catalyst 2nd Generation (2.73 mg, 4.338 μmol). The reaction mixture was warmed to 90° C. and stirred for 35 min, after which time the completed reaction was cooled to room temperature. The mixture was concentrated and purified on a prep TLC silica gel plate eluting with 75% EtOAc in heptane three times to provide compound 244 (16 mg, 0.018 mmol, 41%) (MW_{Calc}+H=893.43; MW_{Obs}=894.46) after scraping off the desired fraction, eluting the product off the silica gel with 10% MeOH in EtOAc (4×5 mL ea) via filtration, concentration of the filtrate, and drying under vacuum.

To a stirred solution of the protected macrocycle (244, 15 mg, 0.017 mmol) in DCM (0.105 ml) at room temperature was added TFA (0.105 ml, 1.363 mmol). The reaction mixture was stirred for 7 min, after which time it was concentrated, azeotroped to dry with toluene (2×5 mL ea) and dried under vacuum. The resultant oily residue was dissolved with stirring in MeCN (0.3 mL) at room temperature followed by the addition of 2-(4-cyclohexyl-1H-1,2,3-triazol-1-yl)acetic acid (5.27 mg, 0.025 mmol), triethylamine (9.35 μL, 0.067 mmol) and HATU (9.57 mg, 0.025 mmol). The reaction mixture was stirred for 1 h, after which time water (1 mL) was added, and the resultant mixture was extracted with EtOAc (3×3 mL ea). The combined organic layers were washed with water (1 mL), brine (1 mL), dried over Na₂SO₄, filtered, concentrated, and dried under vacuum to provide the desired amide intermediate. (17 mg, 0.017 mmol, 100+%).

To a stirred solution of the above amide in EtOAc (1.5 ml) and ethanol (1.5 ml) at room temperature was purged with N₂ atmosphere (3×) followed by the addition of 10% Pd—C(20 mg, 0.188 mmol). The mixture was purged with H₂ (3×) and stirred under a H₂ atmosphere under balloon pressure for 18 h. The completed reaction was purged with N₂ atmosphere (3×) followed by filtering over a pad Celite (3 g) eluting with 1:1 EtOH in EtOAc (2×5 mL ea), and EtOAc (10 mL). The combined filtrates were concentrated, and azeotroped to dry with MeOH (2×5 mL ea) to provide the crude acid as a film. The residue was dissolved with stirring in methanol (0.6 mL) at room temperature followed by the addition of 1N sodium hydroxide (0.3 ml, 0.30 mmol). The final reaction mixture was stirred for 30 min, followed by neutralization with 1N HCl (0.3 mL, 0.3 mmol), and purified by HPLC to provide A-344 (6.8 mg, 0.009 mmol, 52%) (MW_{Calc}+H=771.42; MW_{Obs}=771.60) after combining the desired fractions, concentration, and drying under vacuum.

### Preparation of A-345 to A-347

A-345 was prepared in a similar fashion to A-344 using 5-hexenoic acid instead of 6-heptenoic acid to form the analog of 241 with one less methylene. Using the same sequence of reactions ultimately provides A-345 (6.8 mg, 0.009 mmol) (MW_{Calc}+Na=781.41; MW_{Obs}=781.31).

A-346 was prepared in a similar fashion to A-344 using tert-butyl (3-bromobutyl)carbamate instead of tert-butyl (3-bromopropyl)carbamate, and 5-hexenoic acid instead of 6-heptenoic acid to form an alternative analog of 241. Using the same sequence of reactions ultimately provides A-346 (4.0 mg, 0.005 mmol) (MW_{Calc}+H=771.42; MW_{Obs}=771.76).

A-347 was prepared in a similar fashion to A-345 using tert-butyl (3-bromoethyl)carbamate instead of tert-butyl (3-bromopropyl)carbamate, and 7-octenoic acid instead of 6-heptenoic acid to form an alternative analog of 241. Using the same sequence of reactions ultimately provides A-347 (16 mg, 0.021 mmol) (MW_{Calc}+H=771.42; MW_{Obs}=771.40).

### Preparation of A-348, A-349, and A-350

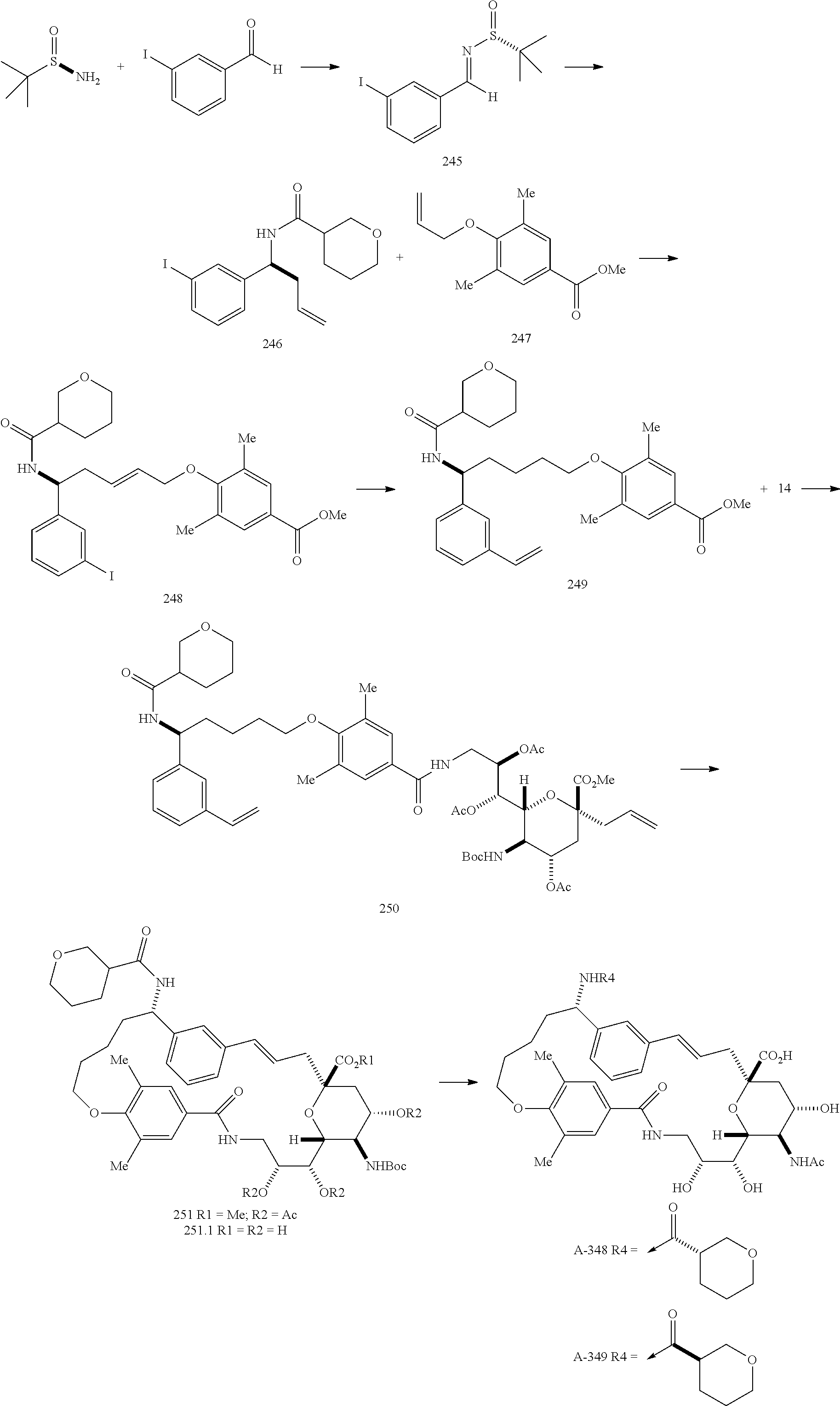

To a stirred solution of R-(+)-tert-butansulfinamide (0.575 g, 4.741 mmol) in DCM (20.00 mL) at room temperature was added magnesium sulfate (5.19 g, 43.10 mmol), p-toluenesulfonic acid monohydrate (0.041 g, 0.216 mmol), and then m-iodobenzaldehyde (1.00 g, 4.31 mmol). The reaction suspension was stirred for 16 h, after which time it was diluted with DCM (30 mL) and filtered over a pad of Celite (5 g) eluting with DCM (2×10 mL ea). The filtrate was concentrated, and over a Biotage Ultra SNAP column (10 g) eluting with 10 CV gradient of 0 to 50% EtOAc in heptane to provide compound 245 (1.4 g, 4.18 mmol, 97%) after combining the desired fractions, concentration, and drying under vacuum.

To a stirred solution of (R,E)-N-(3-iodobenzylidene)-2-methylpropane-2-sulfinamide (245, 1.37 g, 4.087 mmol) in THF (5 mL) at −78° C. was added dropwise 1 M allylmagnesium bromide in ethyl ether (5.11 ml, 5.109 mmol) over 5 min. The mixture was warmed to room temperature and stirred for 4 h. The completed reaction was quench with sat NH₄Cl, and then extracted with DCM (3×10 mL ea). The combined organic layers were washed with brine (2 mL) then dried over Na₂SO₄, filtered and concentrated to dry. The residue was purified over a Biotage Ultra SNAP column (25 g) eluting with 10 CV gradient of 0 to 100% EtOAc in heptane to provide the desired product in ca 6:1 dr of (R)—N—((S)-1-(3-iodophenyl)but-3-en-1-yl)-2-methylpropane-2-sulfinamide (1.4 g, 3.71 mmol, 91%) after combining the desired fractions, concentration, and drying under vacuum.

To a stirred solution of (R)—N—((S)-1-(3-iodophenyl)but-3-en-1-yl)-2-methylpropane-2-sulfinamide (1.4 g, 3.71 mmol) in THF (7 mL) at 0° C. was slowly added conc. HCl (1.725 mL, 20.435 mmol) follow by warming to room temperature. The reaction mixture was stirred for 5 h, after which time the reaction was slowly quenched with sat. NaHCO₃ until a pH>9 was obtained. The mixture was extracted with EtOAc (4×5 mL ea), and the combined organic layers were washed with brine (5 mL) then dried over Na₂SO₄, filtered and concentrated to dry. The crude amine was dissolved with stirring in MeCN (10 mL) at room temperature followed by the addition of triethylamine (1.994 mL, 14.305 mmol), tetrahydro-2H-pyran-4-carboxylic acid (0.665 g, 5.109 mmol), and HATU (2.331 g, 6.131 mmol). The final reaction mixture was stirred at rt for 3 hours, after which time it was diluted with water (5 mL) and extracted with EtOAc (4×5 mL ea). The combined organic layers were dried over Na₂SO₄, filtered and concentrated to dry. The residue was purified over a Biotage Ultra SNAP column (25 g) eluting with 10 CV gradient of 0 to 100% EtOAc in heptane to provide the desired product in ca 6:1 dr of compound 246 (1.30 g, 3.37 mmol, 83%) after combining the desired fractions, concentration, and drying under vacuum.

To a stirred solution of 4-hydroxy-3,5-dimethylbenzoic acid (7 g, 42.125 mmol) in MeOH (50 ml, 1235.883 mmol) at room temperature was added conc. H₂SO₄ (0.449 ml, 8.425 mmol). The reaction mixture was warmed to reflux and stirred for 16 h, after which time it was concentrated. The residue was crystalized from EtOAc (50 mL) to provide methyl 4-hydroxy-3,5-dimethylbenzoate (5.0 g, 27.7 mmol, 66%) after filtering, washing with cold EtOAc (2×5 mL ea), and drying the collected solid under vacuum.

To a stirred solution of methyl 4-hydroxy-3,5-dimethylbenzoate (1.26 g, 6.992 mmol) in DMF (0.2 mL) at room temperature was added potassium carbonate (1.305 g, 9.439 mmol) and 3-bromopropene (1.210 ml, 13.984 mmol). The reaction mixture was stirred for 3 h, after which time it was diluted with water (5 mL) and extracted with EtOAc (3×5 mL ea). The combined organic layers were washed with brine (5 mL), dried over Na₂SO₄, filtered and concentrated to dry. The residue was purified over a Biotage Ultra SNAP column (25 g) eluting with 10 CV gradient of 0 to 100% EtOAc in heptane to provide compound 247 (1.40 g, 6.35 mmol, 91%) (MW_{Calc}+H=221.11; MW_{Obs}=221.20) after combining the desired fractions, concentration, and drying under vacuum.

To a stirred solution of methyl 4-(allyloxy)-3,5-dimethylbenzoate (247. 686 mg, 3.115 mmol) in toluene (4.8 mL) at room temperature was added N—((S)-1-(3-iodophenyl)but-3-en-1-yl)tetrahydro-2H-pyran-3-carboxamide (246, 400 mg, 1.038 mmol), and quinone (8.5 μL, 0.104 mmol) followed by Hoveyda-Grubbs Catalyst 2nd Generation (65.3 mg, 0.104 mmol). The reaction mixture was warmed to 50° C., stirred for 4 h, then cooled to room temperature, and stirred for an additional 18 h. The completed reaction was concentrated and purified over a Biotage Ultra SNAP column (25 g) eluting with 10 CV gradient of 0 to 100% EtOAc in heptane to provide compound 248 (260 mg, 0.450 mmol, 43%) (MW_{Calc}+Na=600.13; MW_{Obs}=600.05) after combining the desired fractions, concentration, and drying under vacuum.

To a stirred solution of methyl 4-(((5S,E)-5-(3-iodophenyl)-5-(tetrahydro-2H-pyran-3-carboxamido)pent-2-en-1-yl)oxy)-3,5-dimethylbenzoate (248, 260 mg, 0.45 mmol) in THF (3.90 mL) at room temperature was added 2-nitrobenzenesulfonohydrazide (489 mg, 2.251 mmol) and triethylamine (628 μL, 4.503 mmol). The reaction vessel was sealed, warmed to 40° C., and stirred for 18 h. The completed reaction was cooled to room temperature, concentrated, and purified directly over a Biotage Ultra SNAP column (10 g) eluting with 10 CV gradient of 0 to 80% EtOAc in heptane to provide the reduced version of compound 248 (210 mg, 0.362 mmol, 80%) after combining the desired fractions, concentration, and drying under vacuum. The resultant pure residue was dissolved with stirring in THF (3.90 mL) at room temperature followed by the addition of silver oxide (522 mg, 2.251 mmol), 2-vinylboronic acid pinacolester (208 mg, 1.351 mmol), and then tetrakis(triphenylphosphine)palladium(0) (52.0 mg, 0.045 mmol). The reaction was warmed to 60° C. and stirred for 2 h. The completed reaction was concentrated and purified directly over a Biotage Ultra SNAP column (10 g) eluting with 10 CV gradient of 0 to 80% EtOAc in heptane to provide compound 249 (140 mg, 0.292 mmol, 81%) (MW_{Calc}+Na=502.27; MW_{Obs}=502.19) after combining the desired fractions, concentration, and drying under vacuum.

To a stirred solution of methyl 3,5-dimethyl-4-(((5S)-5-(tetrahydro-2H-pyran-3-carboxamido)-5-(3-vinylphenyl)pentyl)oxy)benzoate (249, 140 mg, 0.292 mmol) in methanol (1.4 mL) and THF (1.4 mL) at room temperature was added 1 N NaOH (1.46 mL, 1.46 mmol). The reaction was stirred for 16 h, after which time 1 N HCl (2.0 mL, 2.0 mmol) was added followed by the mixture being extracted with EtOAc (3×5 mL ea). The combined organic layers were washed with brine (5 mL), dried over Na₂SO₄, filtered and concentrated to dry to provide the desired crude acid without further purification.

To a stirred solution of (2R,4S,5R,6R)-methyl 2-allyl-6-((1R,2R)-3-amino-1,2-dihydroxypropyl)-5-((tert-butoxycarbonyl)amino)-4-hydroxytetrahydro-2H-pyran-2-carboxylate (14, 100 mg, 0.247 mmol) and 3,5-dimethyl-4-(((5S)-5-(tetrahydro-2H-pyran-3-carboxamido)-5-(3-vinylphenyl)-pentyl)oxy)benzoic acid from the previous reaction (150 mg, 0.321 mmol) in MeCN (1.5 mL) at room temperature was added triethylamine (103 μl, 0.742 mmol), HOBT (18.93 mg, 0.124 mmol), and then by EDC (71.1 mg, 0.371 mmol). The reaction mixture was stirred for 24 h after which time it was diluted with water (3 mL) and NaHCO₃ (3 mL). The resultant mixture was extracted with EtOAc (3×5 mL ea), and the combined organic layers were washed with brine (5 mL), dried over Na₂SO₄, filtered and concentrated to dry. The resulting residue was dissolved with stirring in pyridine (0.5 mL) at room temperature followed by the addition of acetic anhydride (500 μL, 5.299 mmol). The final reaction mixture was stirred for 16 h, after which time it was diluted with EtOAc (10 mL), and then washed with 1N HCl (5 mL), sat. NH₄Cl (5 mL), and brine (5 mL). The organic layer was dried over Na₂SO₄, filtered and concentrated to dry. The residue was purified over a Biotage Ultra SNAP column (10 g) eluting with 10 CV gradient of 0 to 100% EtOAc in heptane to provide compound 250 (120 mg, 0.123 mmol, 50%) (MW_{Calc}+H=978.49; MW_{Obs}=978.46) after combining the desired fractions, concentration, and drying under vacuum.

To a stirred solution of (1R,2R)-1-((2R,3R,4S,6R)-4-acetoxy-6-allyl-3-((tert-butoxycarbonyl)amino)-6-(methoxycarbonyl)tetrahydro-2H-pyran-2-yl)-3-(3,5-dimethyl-4-(((5S)-5-(tetrahydro-2H-pyran-3-carboxamido)-5-(3-vinylphenyl)pentyl)oxy)benzamido)propane-1,2-diyl diacetate (250, 110 mg, 0.112 mmol) in toluene (110 mL) at room temperature was added quinone (3.69 μL, 0.045 mmol) and Hoveyda-Grubbs Catalyst 2nd Generation (17.67 mg, 0.028 mmol). The reaction mixture was warmed to 80° C. and stirred for 1 h. The completed reaction was cooled to room temperature, concentrated, and purified on 4 prep TLC silica gel plates eluting with 90% EtOAc in heptane to provide compound 251 (31 mg, 0.033 mmol, 29%. Rf=0.5) (MW_{Calc}+H=950.46; MW_{Obs}=950.41) after scraping off the desired fraction, eluting the product off the silica gel with 10% MeOH in EtOAc (4×5 mL ea) via filtration, concentration of the filtrate, and drying under vacuum.

To a stirred solution of the protected macrocycle (251, 30 mg, 0.032 mmol) in THF (947 μL) and methanol (947 μL) at room temperature was added 1 N sodium hydroxide (947 μl, 0.947 mmol). The reaction mixture was stirred for 28 h, after which time 1N HCl (1.5 mL) and sat NaCl 1 mL were added followed by extracting with EtOAc (3×5 mL ea). The combined organic layers were washed with brine (5 mL), and then dried over Na₂SO₄, filtered and concentrated to dry to provide the crude acid 251.1, which was divided in two portions for the subsequent reactions.

Half of the crude acid from above was dissolved in DCM (158 μL) at room temperature followed by the addition of TFA (158 μl, 2.056 mmol). The reaction was stirred for 12 min, after which time it was concentrated, and azeotroped to dry with toluene (3×5 mL ea). The resulting amine was dissolved with stirring in THF (330 μL) at room temperature followed by the addition of 10% aq sodium bicarbonate (117 mg, 0.139 mmol), and then acetic anhydride (10.49 μL, 0.111 mmol). The reaction mixture was stirred for 15 min, after which time it was acidified with conc HCl (14.07 μL, 0.167 mmol) to a pH<2. The mixture was diluted with MeOH (1.5 mL), and purified directly by HPLC to provide A-348 (4 mg, 0.005 mmol, 34%, RT=5.873 min) (MW_{Calc}+H=752.37; MW_{Obs}=752.31), and A-349 (4 mg, 0.005 mmol, 34%, RT=5.999 min) (MW_{Calc}+H=752.37; MW_{Obs}=752.31) after combining the desired fractions, concentration, and drying under vacuum. The yields are based on using 50% of the crude acid above.

NOTE: HPLC method: Column=YMC Pack Pro C18 RS (5 μm, 20×150 mm); Flow rate=20 mL/min

| | % Water | % Acetonitrile |
|---|---|---|
| Time (min) | (with 0.1% formic acid) | (with 0.1% formic acid) |
| 0 | 85 | 15 |
| 1.5 | 85 | 15 |
| 9 | 1 | 99 |
| 9.9 | 1 | 99 |
| 10 | 85 | 15 |
| 11.5 | 85 | 15 |

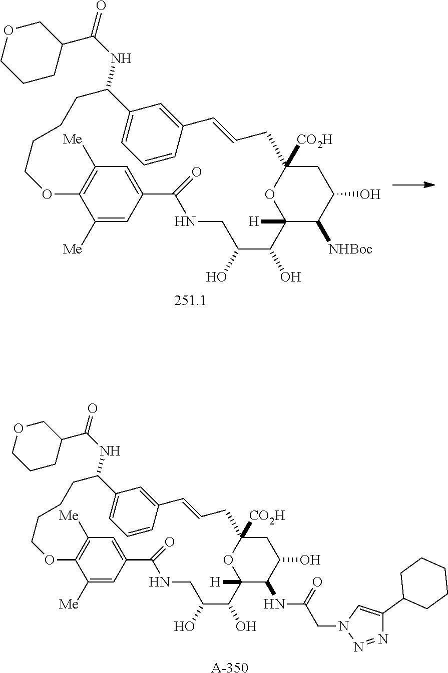 [SMILES: Cc1cc2cc(C)c1OCCCC[C@H](NC(=O)C1CCCOC1)c1cccc(c1)/C=C/C[C@]1(C(=O)O)C[C@H](O)[C@@H](NC(=O)OC(C)(C)C)[C@@H](O1)[C@H](O)[C@H](O)CNC2=O ; Cc1cc2cc(C)c1OCCCC[C@H](NC(=O)C1CCCOC1)c1cccc(c1)/C=C/C[C@]1(C(=O)O)C[C@H](O)[C@@H](NC(=O)Cn3cc(C4CCCCC4)nn3)[C@@H](O1)[C@H](O)[C@H](O)CNC2=O ; "A-350"]

To a stirred solution of the second half of the crude acid from above (251.1, 13 mg, 0.016 mmol) in DMF (156 μL) at room temperature was added potassium carbonate (8.87 mg, 0.064 mmol) and allyl bromide (3.47 μL, 0.04 mmol). The reaction mixture was stirred for 45 min, after which time it was diluted with water (2 mL) and extracted with EtOAc (2×5 mL). The combined EtOAc layers were washed with brine (2 mL), dried over Na₂SO₄, filtered, and concentrated. The crude ester was dissolved with stirring in pyridine (78 μL, 0.964 mmol) followed by the addition of acetic anhydride (78 μl, 0.827 mmol) and DMAP (1.0 mg, 0.008 mmol). The reaction mixture was stirred for 6 h, after which time it was diluted with EtOAc (5 mL), and washed with 1N HCl (2 mL), NH4C1 (2 mL), and brine (2 mL). The organic layer was dried over Na₂SO₄, filtered and concentrated to dry. The crude product (15 mg, 0.015 mmol, 96%) was used in the next step without further purification.

To a stirred solution of the crude protected macrolide (15 mg, 0.015 mmol) in DCM (0.3 mL) at room temperature was added TFA (0.3 mL, 3.92 mmol). The reaction was stirred for 10 min, after which time it was concentrated, azeotroped to dry with toluene (2×5 mL ea), and dried under vacuum for 30 min. The residue was dissolved with stirring in MeCN (0.3 mL) at room temperature follow by the addition of 2-(4-cyclohexyl-1H-1,2,3-triazol-1-yl)acetic acid (10.29 mg, 0.049 mmol), triethylamine (13.71 μl, 0.098 mmol), and then HATU (9.35 mg, 0.025 mmol). The reaction mixture was stirred for 30 min, after which time it was quenched with sat NaHCO₃ (3 mL) followed by the addition of EtOAc (6 mL). The organic layer was washed NaHCO₃ (3 mL), and brine (3 mL), dried over Na₂SO₄, filtered, concentrated, and dried under vacuum. The crude product was dissolved with stirring in DCM (1 mL) at room temperature followed by the addition of phenylsilane (10.1 μl, 0.082 mmol) and then Pd(Ph₃P)₄ (2.0 mg, 1.7 mmol). The reaction mixture was stirred for 1 h, after which time MeOH (2 mL) was added followed by concentration, and azeotroping to dryness with MeOH (2 mL) then toluene (2 mL). The penultimate intermediate was dissolved with stirring in MeOH (0.6 mL) at room temperature followed by the addition of potassium carbonate (9.06 mg, 0.066 mmol). The reaction was stirred for 20 min, and then 1N NaOH (0.1 mL, 0.10 mmol) was added followed by stirring for an additional 1 h. The completed reaction was quenched with 1 N HCl (164 μl, 0.164 mmol), diluted with MeOH (1 mL), and purified by HPLC to provide A-350 (2 mg, 0.002 mmol, 15% overall) (MW_{Calc}+H=901.47; MW_{Obs}=901.40) after combining the desired fractions, concentration, and drying under vacuum.

### Preparation of A-351 to A-353

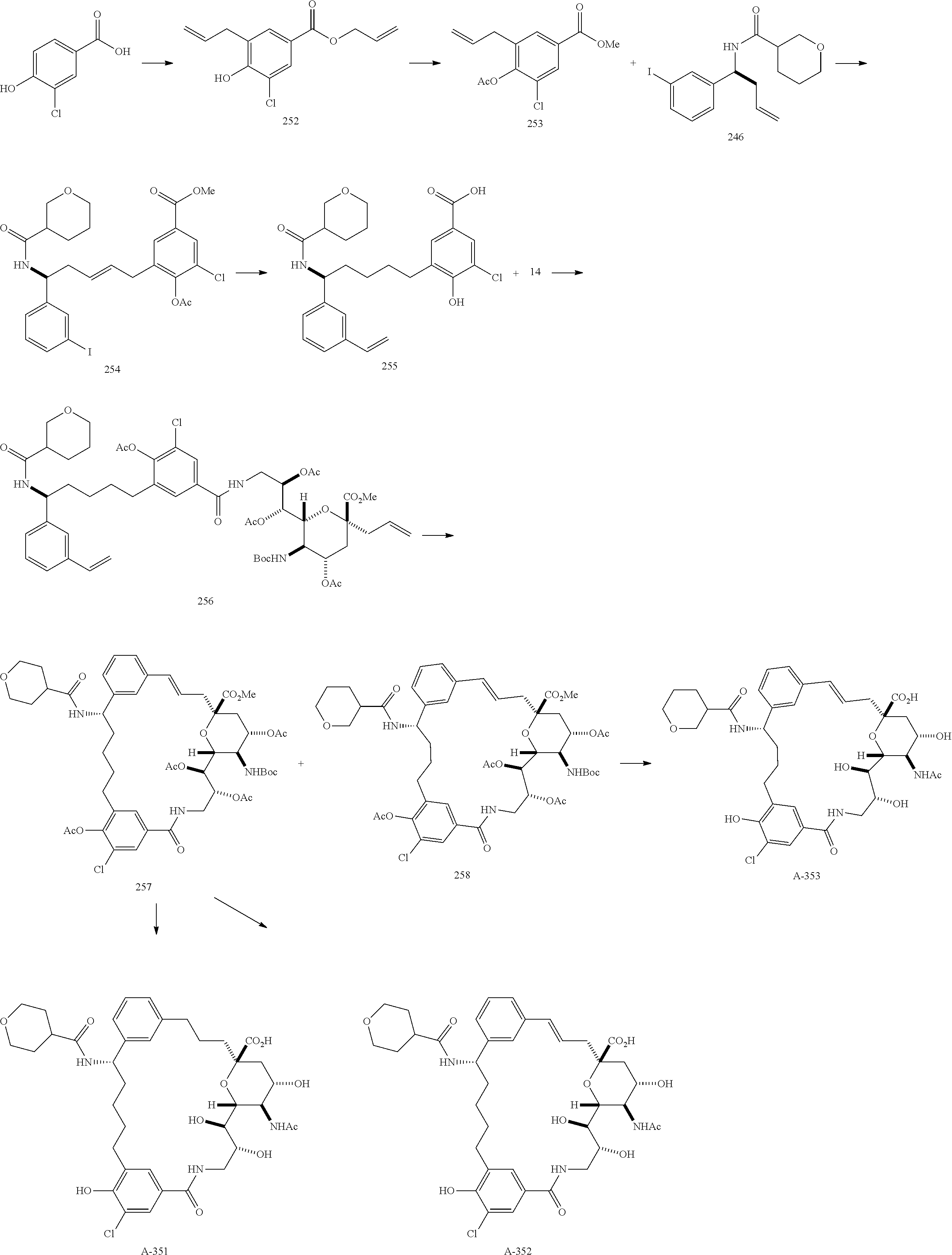

To a stirred solution of 3-chloro-4-hydroxybenzoic acid (5.50 g, 31.87 mmol) in DMF (60 mL) at room temperature were added potassium carbonate (15.42 g, 111.552 mmol) and allyl bromide (8.27 mL, 95.62 mmol). The reaction mixture was stirred for 16 h, after which time it was diluted with water (120 mL) and extracted with 1:1 mixture of MTBE to EtOAc (3×100 mL ea). The combined organic layers were washed with water (5×20 mL ea), dried over Na2SO4, filtered, and concentrated to provide a crystalline solid (8.0 g, 31.7 mmol). The solid was divided in eight reaction vials (1 g ea, 3.96 mmol ea) followed by the addition of dimethylaniline (1 mL ea, 8.25 mmol ea). The vials were sealed in heated at 250° C. via a microwave apparatus for 30 min. The completed reactions were cooled to room temperature, combined, and purified over a Biotage Ultra SNAP column (150 g) eluting with 2 CV of heptane, a 7 CV gradient of 0 to 2% EtOAc in heptane, 2 CV of 2% EtOAc, a 5 CV gradient of 2 to 10% EtOAc in heptane, 1 CV 10% EtOAc in heptane, and finally a 3 CV gradient of 10 to 25% EtOAc in heptane to provide compound 252 (6.70 g, 26.5 mmol, 83%) after combining the desired fractions, concentration, and drying under vacuum.

To a stirred solution of allyl 3-allyl-5-chloro-4-hydroxybenzoate (252, 1.2 g, 4.749 mmol) in pyridine (1.92 ml) at room temperature was added DMAP (0.058 g, 0.475 mmol) and acetic anhydride (1.792 ml, 18.995 mmol). The reaction mixture was stirred for 16 h, after which time it was diluted with EtOAc (50 mL), the resultant mixture was washed with 1N HCl (3×30 mL ea), and then washed with brine (20 mL). The organic layer was dried over Na₂SO₄, filtered and concentrated to dry to provide a clean oil (1.4 g, 4.75 mmol, 100%). The oil was dissolved with stirring in DCM (18.00 mL) at room temperature followed by the addition of Pd(Ph₃P)₄ (0.198 g, 0.171 mmol) and phenylsilane (1.758 ml, 14.247 mmol). The reaction mixture was stirred for 2 h, after which time it was diluted with EtOAc (10 mL) and MeOH (10 mL), followed by concentration to dry. The residue was purified over a Biotage Ultra SNAP column (25 g) eluting with a 10 CV gradient of 0 to 100% EtOAc in heptane to provide the acid intermediate (1.02 g, 4.01 mmol, 84%) after combining the desired fractions, concentration, and drying under vacuum.

To a stirred solution of 4-acetoxy-3-allyl-5-chlorobenzoic acid (840 mg, 3.30 mmol) in methanol (2.67 mL) and toluene (12.6 mL) at room temperature was added 2 M TMS-diazomethane in DCM (4.95 μL, 9.90 mmol). The reaction was stirred for 30 min, after which time it was carefully concentrated, and purified directly over a Biotage Ultra SNAP column (25 g) eluting with a 10 CV gradient of 0 to 50% EtOAc in heptane to provide compound 253 (890 mg, 3.30 mmol, 100%) after combining the desired fractions, concentration, and drying under vacuum.

To a stirred solution of methyl 4-acetoxy-3-allyl-5-chlorobenzoate (253, 942 mg, 3.504 mmol) and N—((S)-1-(3-iodophenyl)but-3-en-1-yl)tetrahydro-2H-pyran-3-carboxamide (14, 450 mg, 1.168 mmol) in toluene (5.40 mL) at room temperature was added Hoveyda-Grubbs Catalyst 2nd Generation (73.4 mg, 0.117 mmol). The reaction was warmed to 50° C. and stirred for 18 h. The reaction was cooled to room temperature, concentrated, and purified directly over a Biotage Ultra SNAP column (25 g) eluting with a 10 CV gradient of 0 to 100% EtOAc in heptane to provide compound 254 (392 mg, 0.626 mmol, 54%) (MW_{Calc}+H=626.07; MW_{Obs}=626.05) after combining the desired fractions, concentration, and drying under vacuum.

To a stirred solution of methyl 4-acetoxy-3-chloro-5-((5S,E)-5-(3-iodophenyl)-5-(tetrahydro-2H-pyran-3-carboxamido)pent-2-en-1-yl)benzoate (380 mg, 0.607 mmol) in THF (7.60 mL) was added triethylamine (846 μl, 6.071 mmol) followed by 2-nitrobenzenesulfonohydrazide (659 mg, 3.036 mmol). The reaction mixture was warmed to 40° C., stirred for 18, after which time additional triethylamine (300 μl, 2.15 mmol) and 2-nitrobenzenesulfonohydrazide (300 mg, 1.38 mmol) were added followed by stirring at 40° C. for 8 h. The completed intermediate reaction was cooled to room temperature, concentrated, diluted in pyridine (1.92 ml) at room temperature followed by the addition of DMAP (0.02 g, 0.165 mmol) and acetic anhydride (1.0 ml, 10.58 mmol). The reaction mixture was stirred for 16 h, after which time it was diluted with EtOAc (20 mL), the resultant mixture was washed with 1N HCl (3×10 mL ea), and then washed with brine (5 mL). The organic layer was dried over Na₂SO₄, filtered, concentrated, and purified directly over a Biotage Ultra SNAP column (25 g) eluting with a 10 CV gradient of 0 to 100% EtOAc in heptane to provide the unsaturated intermediate (335 mg, 0.534 mmol, 88%) after combining the desired fractions, concentration, and drying under vacuum. (MW_{Calc}+H=586.08; MW_{Obs}=585.99).

To a stirred solution of methyl 4-acetoxy-3-chloro-5-((5S)-5-(3-iodophenyl)-5-(tetrahydro-2H-pyran-3-carboxamido)pentyl)benzoate (254, 335 mg, 0.534 mmol) in THF (5.70 mL) were added silver oxide (618 mg, 2.668 mmol), 2-vinylboronic acid pinacolester (247 mg, 1.601 mmol), and Pd(PPh₃)₄ (61.7 mg, 0.053 mmol). The reaction mixture was warmed to 60° C. and stirred for 1 h. The completed reaction was cooled to room temperature, diluted with EtOAc (15 mL), filtered over a pad of Celite (5 g), eluting with EtOAc (2×5 mL ea), and concentrated. The residue was purified over a Biotage Ultra SNAP column (25 g) eluting with a 12 CV gradient of 0 to 66% EtOAc in heptane to provide the vinyl intermediate (287 mg, 0.543 mmol, 100+%) after combining the desired fractions, concentration, and drying under vacuum. The resultant ester from above was dissolved with stirring in THF (5.70 mL) and MeOH (6.0 mL) at room temperature followed by the addition of 1 N NaOH (2.13 mL, 2.13 mmol), and LiOH (89 mg, 3.735 mmol). The reaction mixture was warmed to 97° C. and stirred for 3 h. The completed reaction was cooled to room temperature, acidified with 1 N HCl (5 mL), and then extracted with EtOAc (3×10 mL ea). The combined organic layers were washed with brine (10 mL), dried over Na₂SO₄, filtered, concentrated, and purified over a Biotage Ultra SNAP column (25 g) eluting with a 10 CV gradient of 0 to 100% EtOAc in heptane to provide 255 (224 mg, 0.475 mmol, 89%) (MW_{Calc}+Na=494.18; MW_{Obs}=494.12) after combining the desired fractions, concentration, and drying under vacuum.

To a stirred solution of (2R,4S,5R,6R)-methyl 2-allyl-6-((1R,2R)-3-amino-1,2-dihydroxypropyl)-5-((tert-butoxycarbonyl)amino)-4-hydroxytetrahydro-2H-pyran-2-carboxylate (14, 120 mg, 0.297 mmol) and 3-chloro-4-hydroxy-5-((5S)-5-(tetrahydro-2H-pyran-3-carboxamido)-5-(3-vinylphenyl)pentyl)-benzoic acid (255, 168 mg, 0.356 mmol) in MeCN (1.80 mL) was added triethylamine (124 μl, 0.89 mmol), HOBT (22.72 mg, 0.148 mmol), and then EDC (77 mg, 0.401 mmol). The reaction was stirred for 24 h, after which time EDC (35 mg, 0.18 mmol) was added. The final mixture was stirred for 8 h, after which time water (3 mL) and NaHCO₃ (3 mL) were added. The mixture was extracted with EtOAc (3×10 mL), and the combined organic layers were washed with 1N HCl (10 mL), sat NH₄Cl (10 mL), and brine (10 mL). The organic layer was dried over Na₂SO₄, filtered, and concentrated to an oil. The crude oil was dissolved with stirring in pyridine (600 μL) at room temperature followed by the addition of acetic anhydride (600 μl, 6.359 mmol). The reaction mixture was stirred for 18 h, after which time the mixture was diluted with EtOAc (20 mL), and then washed with 1N HCl (2×5 mL ea), NH₄Cl (5 mL), and finally brine (5 mL). The organic layer was dried over Na₂SO₄, filtered, concentrated, and purified over a Biotage Ultra SNAP column (10 g) eluting with a 3 CV gradient of 0 to 100% EtOAc in heptane, and then 6CV of EtOAc to provide 256 (210 mg, 0.205 mmol, 69%) (MW_{Calc}+H=1026.43; MW_{Obs}=1026.42) after combining the desired fractions, concentration, and drying under vacuum.

To a stirred solution of (1R,2R)-3-(4-acetoxy-3-chloro-5-((5S)-5-(tetrahydro-2H-pyran-3-carboxamido)-5-(3-vinylphenyl)pentyl)benzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (256, 200 mg, 0.195 mmol) in toluene (150 mL) at room temperature were added quinone (7.99 μl, 0.097 mmol) and Hoveyda-Grubbs Catalyst 2nd Generation (30.6 mg, 0.049 mmol). The reaction mixture was warmed to 88° C. and stirred for 30 min, after which time it was cooled to room temperature followed by diluting with MeOH (3 mL) and EtOAc (3 mL). The mixture was concentrated and purified on 4 prep TLC silica gel plates eluting with 60% EtOAc in heptane to provide compound 257 (110 mg, 0.110 mmol, 57%, Rf=0.3) (MW_{Calc}+H=998.40; MW_{Obs}=998.39) and 258 (30 mg, 0.030 mmol, 16%, Rf=0.24) (MW_{Calc}+H=984.38; MW_{Obs}=984.33) after scraping off the desired fractions separately, eluting the products off the silica gel with 10% MeOH in EtOAc (4×5 mL ea) via filtration, concentration of the filtrate, and drying under vacuum.

To a stirred solution of the fully protected macrocycle (257, 14 mg, 0.014 mmol) in EtOH (1 mL) and EtOAc (1 mL) at room temperature and with purged with a N₂ atmosphere (3×) was added 10% Pd—C (14 mg, 0.132 mmol). The mixture was purged with a H₂ atmosphere (5×), and then placed under a H₂ atmosphere under balloon pressure and stirred for 18 h. The completed reaction was purged with a N₂ atmosphere (5×) followed by the addition of Celite (2 g) and filtering the suspension over a plug of Celite (5 g), eluting with (5×5 mL ea). The filtrate was concentrated, and azeotroped to dry with MeOH (3×10 mL ea). The resulting residue was dissolved with stirring in methanol (0.421 mL) at room temperature followed by the addition of 1 N NaOH (0.421 ml, 0.421 mmol). The reaction mixture was stirred for 48 h, after which time it was neutralized with 1N HCl (1 mL) and extracted with EtOAc (3×5 mL ea). The combined organic layers were washed with brine (3 mL), dried over Na₂SO₄, filtered, and concentrated. The resulting residue was dissolved with stirring in DCM (0.3 mL) at room temperature followed by the addition of TFA (0.3 mL, 3.92 mmol). The penultimate reaction mixture was stirred for 10 minutes, after which time the completed reaction was concentrated, and azeotroped to dry with toluene (2×10 mL ea). The crude amine residue was dissolved with stirring in THF (0.2 mL) at room temperature followed by the addition of 10% sodium bicarbonate (118 mg, 0.14 mmol) and acetic anhydride (10.58 μL, 0.112 mmol). The final reaction mixture was stirred for 15 min, after which time it was diluted with MeOH (0.1 mL) followed by 1N NaOH (0.1 mL). The mixture was stirred for 30 min, and then acidified with 2-3 drops conc HCl. The mixture was diluted with MeOH (2 mL) and purified directly by HPLC to provide A-351 (7 mg, 0.009 mmol, 66%) (MW_{Calc}+H=760.32; MW_{Obs}=760.37) after combining the desired fractions, concentration, and drying under vacuum.

A-352 was prepared in a similar fashion to A-351 starting with 257) (27 mg, 0.027 mmol) to provide A-352 (8.9 mg, 0.012 mmol, 44%) (MW_{Calc}+H=744.24; MW_{Obs}=744.16).

A-353 was prepared in a similar fashion to A-351 (with the exception of not performing the reduction step of H₂ atmosphere in the presence of Pd/C) starting with 258(17 mg, 0.017 mmol) to provide A-353 (7.0 mg, 0.009 mmol, 54%) (MW_{Calc}+H=758.30; MW_{Obs}=758.28).

### Preparation of A-354 to A-356

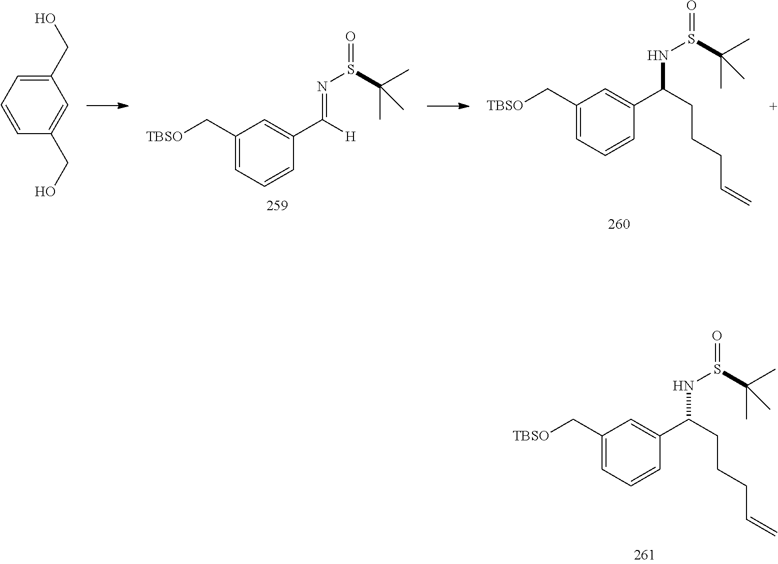 [SMILES: OCc1cccc(CO)c1 ; CC(C)(C)[S@](=O)/N=C/c1cccc(CO[Si](C)(C)C(C)(C)C)c1 ; C=CCCC[C@H](N[S@@](=O)C(C)(C)C)c1cccc(CO[Si](C)(C)C(C)(C)C)c1 ; C=CCCC[C@@H](N[S@@](=O)C(C)(C)C)c1cccc(CO[Si](C)(C)C(C)(C)C)c1]

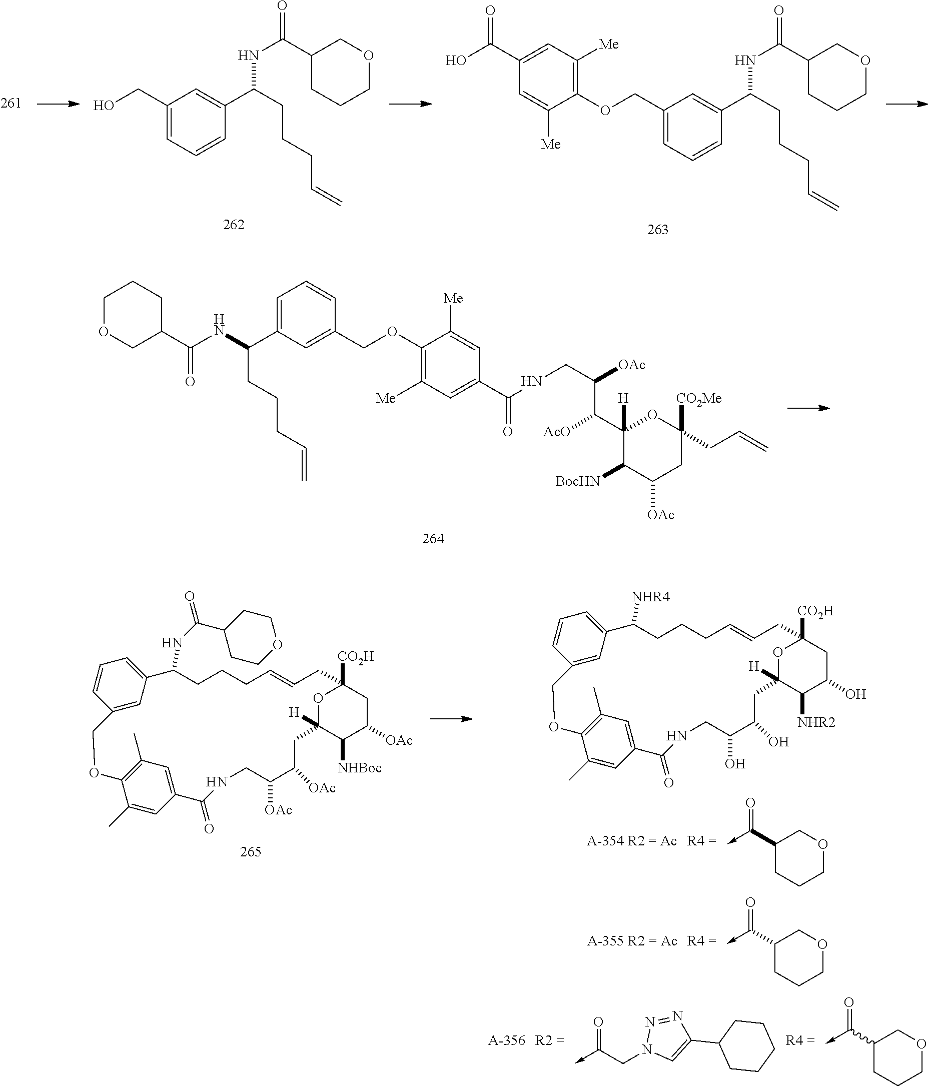

To a stirred solution of 1,3-phenylenedimethanol (10.00 g, 72.38 mmol) in THF (100 mL) at 0° C. was added 60% NaH (2.89 g, 72.378 mmol) and then TBS-CI (10.91 g, 72.38 mmol). The reaction was stirred at 0° C. for 2 h, after which time the mixture was slowly quenched with sat NH₄Cl (30 mL) and extracted with EtOAc (3×30 mL ea). The combined organic layers were dried over Na₂SO₄, filtered, and concentrated. The residue was purified over silica gel (100 g) eluting with 30% EtOAc in heptane to provide 3-(((tert-butyldimethylsilyl)oxy)methyl)phenyl)methanol (11.00 g, 43.60 mmol, 60%) after combining the desired fractions, concentration, and drying under vacuum.

To a stirred solution of (3-(((tert-butyldimethylsilyl)oxy)methyl)phenyl)methanol (2.00 g, 7.92 mmol) in DCM (40.0 mL) at room temperature were added sodium bicarbonate (3.33 g, 39.616 mmol) and Dess-Martin periodinane (4.03 g, 9.51 mmol). The reaction mixture was stirred for 1 h, after which time it was quenched with sat. Na₂S₂O₄ (10 mL) and NaHCO₃ (10 mL). The mixture was extracted with EtOAc (3×20 mL ea), and the combined organic layers were dried over Na₂SO₄, filtered, and concentrated. The residue was purified over a Biotage Ultra SNAP column (50 g) eluting with a 10 CV gradient of 5 to 20% EtOAc in heptane to provide the intermediate aldehyde (1.20 g, 4.79 mmol, 61%) after combining the desired fractions, concentration, and drying under vacuum. To a stirred solution of (S)-2-methylpropane-2-sulfinamide (1.056 g, 8.72 mmol) in DCM (40.0 mL) at room temperature were added magnesium sulfate (9.54 g, 79.23 mmol), p-toluenesulfonic acid monohydrate (0.075 g, 0.40 mmol), followed by the above aldehyde (1.2 g). The reaction mixture was stirred for 24 h, after which time it was diluted with DCM (30 mL) and filtered over a pad of Celite (10 g) eluting with DCM (3×10 mL ea). The combined filtrates were concentrated and purified over a Biotage Ultra SNAP column (25 g) eluting with a 10 CV gradient of 5 to 20% EtOAc in heptane to provide the compound 259 (2.00 g, 5.66 mmol, 71%) (MW_{Calc}+Na=376.18; MW_{Obs}=376.18) as an oil after combining the desired fractions, concentration, and drying under vacuum.

To a stirred solution of (S,E)-N-(3-(((tert-butyldimethylsilyl)oxy)methyl)benzylidene)-2-methylpropane-2-sulfinamide (259, 1.0 g, 2.83 mmol) in DCM (12.00 mL) at −40° C. was slowly added 2 M pent-4-en-1-yl magnesium bromide (11.31 mL, 5.656 mmol) over 5 min. The reaction mixture was stirred at −40° C. for 6 h, after which time it was carefully quenched with aq NH₄CL (10 mL) and stirred at room temperature for an additional 1 h. The mixture was treated with 1 N HCl (10 mL), diluted with EtOAC (30 mL), the layers separated, and the organic layer was washed with brine (20 mL). The organic layer was dried over Na₂SO₄, filtered, and concentrated. The residue was purified over a Biotage Ultra SNAP column (25 g) eluting with a 10 CV gradient of 5 to 35% EtOAc in heptane to provide compound 260 or (S)—N—((S)-1-(3-(((tert-butyldimethylsilyl)oxy)methyl)phenyl)-hex-5-en-1-yl)-2-methylpropane-2-sulfinamide (0.45 g, 1.062 mmol, 38%) (MW_{Calc}+Na=446.26; MW_{Obs}=446.25), and compound 261 or (S)—N—((R)-1-(3-(((tert-butyldimethylsilyl)oxy)-methyl)phenyl)hex-5-en-1-yl)-2-methylpropane-2-sulfinamide (0.70 g, 1.652 mmol, 58%) (MW_{Calc}+Na=446.26; MW_{Obs}=446.22) after combining the desired fractions, concentration, and drying under vacuum.

To a stirred solution of ((S)—N—((R)-1-(3-(((tert-butyldimethylsilyl)oxy)methyl)phenyl)hex-5-en-1-yl)-2-methylpropane-2-sulfinamide (261, 0.7 g, 1.652 mmol) in THF (4.90 mL) at 0° C. was added conc HCl (0.697 mL, 8.26 mmol). The reaction mixture was slowly warmed to room temperature and stirred for 5 h. The completed reaction was concentrated, and then dissolved with stirring in MeCN (15 mL) at room temperature followed by the addition of tetrahydro-2H-pyran-3-carboxylic acid (0.258 g, 1.982 mmol), HOBT (0.253 g, 1.652 mmol), EDC (0.412 g, 2.148 mmol), and then triethylamine (1.382 ml, 9.912 mmol). The reaction mixture was stirred for 5 h, after which time the completed reaction was diluted with EtOAc (20 mL). The mixture was washed with water (10 mL), brine (10 mL), and then dried over Na₂SO₄, filtered, and concentrated. The residue was purified over a Biotage Ultra SNAP column (10 g) eluting with a 10 CV gradient of 40 to 100% EtOAc in heptane, and then 3 CV 10% MeOH in EtOAc to provide compound 262 (0.36 g, 1.134 mmol, 69%) (MW_{Calc}+Na=340.20; MW_{Obs}=340.21) after combining the desired fractions, concentration, and drying under vacuum.

To a stirred solution of N—((R)-1-(3-(hydroxymethyl)phenyl)hex-5-en-1-yl)tetrahydro-2H-pyran-3-carboxamide (262, 0.36 g, 1.134 mmol) and methyl 4-hydroxy-3,5-dimethylbenzoate (0.307 g, 1.701 mmol) in THF (5 mL) at 0° C. were added triphenylphosphine (0.446 g, 1.701 mmol) and DIAD (0.331 ml, 1.701 mmol). The reaction mixture was warmed to room temperature and stirred for 6 h. The completed reaction was concentrated, and then purified directly over a Biotage Ultra SNAP column (10 g) eluting with a 10 CV gradient of 0 to 50% EtOAc in heptane, and then 10% MeOH in EtOAc to provide compound methyl 3,5-dimethyl-4-((3-((1R)-1-(tetrahydro-2H-pyran-3-carboxamido)hex-5-en-1-yl)benzyl)oxy)benzoate (350 mg, 0.730 mmol, 64%) as a white solid after combining the desired fractions, concentration, and drying under vacuum.

To a stirred solution of methyl 3,5-dimethyl-4-((3-((1R)-1-(tetrahydro-2H-pyran-3-carboxamido)hex-5-en-1-yl)benzyl)oxy)benzoate (0.35 g, 0.73 mmol) in THF (3 mL) and MeOH (3 mL) at room temperature was added and 1 N NaOH (3.65 mL, 3.65 mmol). The reaction mixture was stirred for 16 h, after which time it was acidified with 1N HCl (4 mL), added sat NaCl (1 mL), and then extracted the mixture with EtOAc (3×20 mL ea). The combined organic layers were dried over Na₂SO₄, filtered, and concentrated. The residue was purified over a Biotage Ultra SNAP column (10 g) eluting with a 10 CV gradient of 40 to 100% EtOAc in heptane, and then 3 CV 10% MeOH in EtOAc to provide compound 263 (0.3 g, 0.644 mmol, 88%) (MW_{Calc}+H=466.25; MW_{Obs}=466.30) as a solid after combining the desired fractions, concentration, and drying under vacuum.

To a stirred solution of (2R,4S,5R,6R)-methyl 2-allyl-6-((1S,2S)-3-azido-1,2-dihydro0xypropyl)-5-((tert-butoxycarbonyl)amino)-4-hydroxytetrahydro-2H-pyran-2-carboxylate (13, 0.10 g, 0.232 mmol) in THF (1.80 mL) and water (0.17 mL) at 0° C. was added 1N trimethylphosphine (0.70 ml, 0.70 mmol). The reaction mixture was warmed to room temperature and stirred for 16 h. The completed reaction was concentrated, and azeotroped to dry with toluene (2×10 mL ea). The crude amine was dissolved with stirring in MeCN (1.20 mL) at room temperature followed by the addition of 3,5-dimethyl-4-((3-((1R)-1-(tetrahydro-2H-pyran-3-carboxamido)hex-5-en-1-yl)benzyl)oxy)benzoic acid (263, 0.162 g, 0.348 mmol), HOBT (0.036 g, 0.232 mmol), EDC (0.067 g, 0.348 mmol), and triethylamine (0.097 ml, 0.697 mmol). The reaction mixture was stirred for 6 h, after which time it was concentrated to dry. The resultant residue was dissolved with stirring in DCM (1 mL) at room temperature followed by the addition of Et₃N (0.5 mL), DMAP (cat.) and Ac₂O (0.219 ml, 2.323 mmol). The completed reaction was concentrated and purified directly over a Biotage Ultra SNAP column (10 g) eluting with a 10 CV gradient of 40 to 100% EtOAc in heptane, and then 3 CV 10% MeOH in EtOAc to provide a mixture of the desired compound with unreacted 263. The resultant mixture was dissolve in EtOAc (10 mL) and washed with 0.2 N NaOH and sat. NaHCO₃ (3 mL). The combined aqueous layer was extracted with EtOAc (3 mL), and the combined organic layers were dried over Na₂SO₄, filtered, and concentrated to provide 264 (0.16 g, 0.164 mmol, 70%) (MW_{Calc}+H=978.49; MW_{Obs}=978.46) after drying under vacuum.

To a stirred solution of (1R,2R)-1-((2R,3R,4S,6R)-4-acetoxy-6-allyl-3-((tert-butoxycarbonyl)amino)-6-(methoxycarbonyl)tetrahydro-2H-pyran-2-yl)-3-(3,5-dimethyl-4-((3-((1R)-1-(tetrahydro-2H-pyran-3-carboxamido)hex-5-en-1-yl)benzyl)oxy)benzamido)-propane-1,2-diyl diacetate (264, 0.16 g, 0.164 mmol) in toluene (160 mL) at room temperature were added quinone (5.37 μL, 0.065 mmol) and Hoveyda-Grubbs Catalyst 2nd Generation (0.026 g, 0.041 mmol). The reaction mixture was warmed to 90° C. and stirred for 24 h, after which time it was cooled to room temperature, diluted with DMSO (0.3 mL), and stirred for 30 min. The resultant mixture was concentrated to dry, and purified over a Biotage Ultra SNAP column (25 g) eluting with a 10 CV gradient of 20 to 100% EtOAc in heptane, and then a 5 CV gradient of 0 to 10% MeOH in EtOAc to provide compound 265 (0.11 g, 0.116 mmol, 71%) (MW_{Calc}+H=950.46; MW_{Obs}=950.45) after combining the desired fractions, concentration, and drying under vacuum.

To a stirred solution of the protected macrocycle (265, 0.11 g, 0.116 mmol) in THF (3.30 mL) and methanol (3.30 mL) at room temperature was added 1 N NaOH (3.47 mL, 3.47 mmol). The reaction mixture was stirred for 48 h, after which time 1N HCl (3.5 mL) was added followed by brine (1 mL). The resultant mixture was extracted with EtOAc (3×10 mL), and the combined organic layers were washed with brine (5 mL), dried over Na₂SO₄, filtered, and concentrated to dry.

A portion of the crude acid (approx. 25 mg) was dissolved with stirring in DCM (1.32 mL) at room temperature followed by the addition of TFA (0.550 ml, 7.139 mmol). The reaction mixture was stirred for 30 min, after which time the mixture was concentrated, and azeotroped to dry with toluene (3×5 mL ea). The resultant crude amine was dissolved in THF (2.75 mL) at room temperature followed by the addition of 10% aqueous sodium bicarbonate (9.73 mg, 0.116 mmol), and then acetic anhydride (10.92 μL, 0.116 mmol). The completed reaction was acidified with conc HCL to pH<2, diluted with MeOH (1.5 mL), and purified directly by HPLC to provide A-354 (10 mg, 0.013 mmol, 45%) (MW_{Calc}+H=752.37; MW_{Obs}=752.34) and A-355 (10 mg, 0.013 mmol, 45%) (MW_{Calc}+H=752.37; MW_{Obs}=752.36) after combining the desired fractions, concentration, and drying under vacuum.

To a stirred solution of a second portion of the above crude acid (60.0 mg, 0.074 mmol) in DCM (2 mL) at room temperature was added TFA (114 μL, 1.482 mmol). The reaction mixture was stirred for 30 min, after which time the mixture was concentrated, and azeotroped to dry with toluene (2×10 mL ea).

In a separate flask was added 2-(4-cyclohexyl-1H-1,2,3-triazol-1-yl)acetic acid (31.0 mg, 0.148 mmol) in MeCN (0.2 mL) at room temperature followed by the addition of HOBT (22.69 mg, 0.148 mmol), and EDC (28.4 mg, 0.148 mmol). This mixture was stirred for 1 h, after which time a solution of the above crude amine in MeCN (600 μL) was added followed by triethylamine (51.6 μL, 0.37 mmol). The final reaction mixture was stirred for 4 h, after which time it was acidified with 1 N HCL (0.2 mL), diluted with MeOH (1 mL), and purified by HPLC to provide A-356 (40 mg, 0.044 mmol, 60%) (MW_{Calc}+H=901.47; MW_{Obs}=901.51) after combining the desired fractions, concentration, and drying under vacuum.

### Preparation of A-357 and A-358

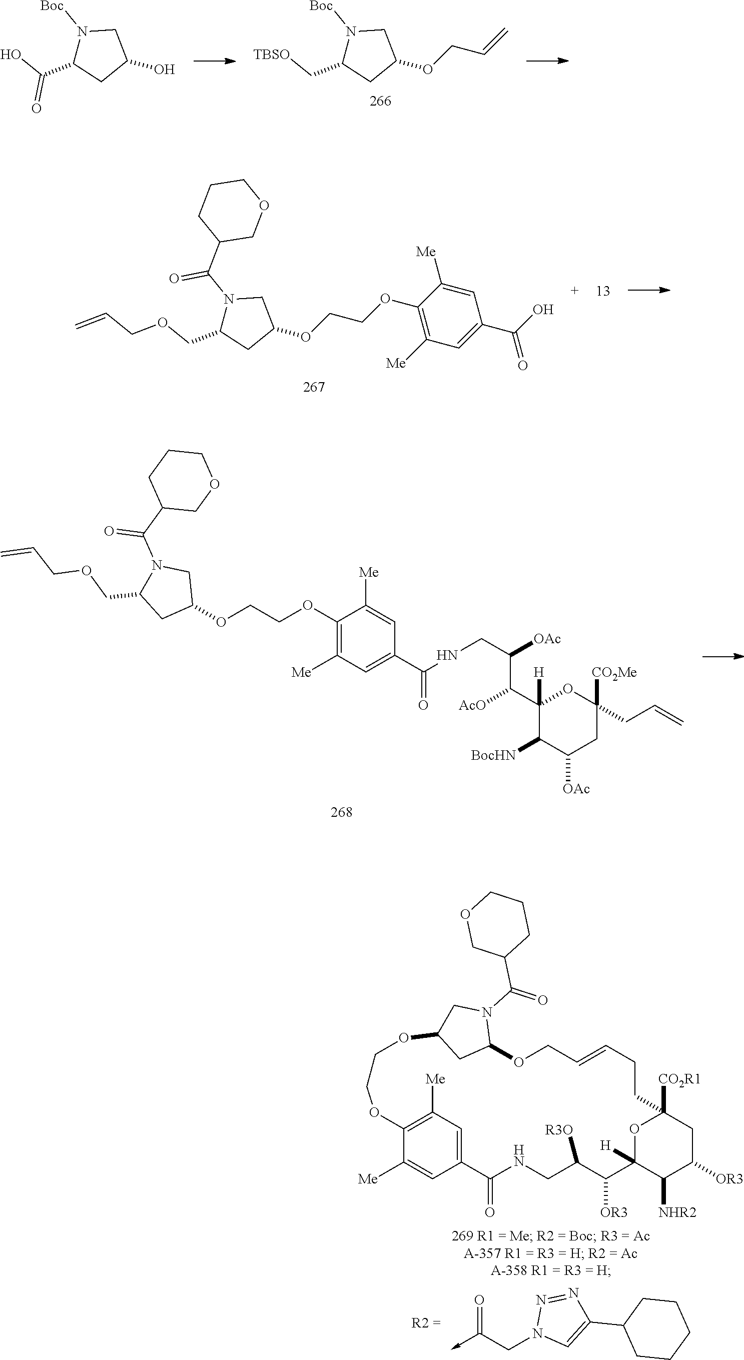

To a stirred solution of N-t-BOC-cis-4-hydroxy-D-proline (5 g, 21.622 mmol) in THF (100 mL) at room temperature under a N₂ atmosphere were added allyl methyl carbonate (5.37 mL, 47.568 mmol) and Pd(Ph₃P)₄ (0.250 g, 0.216 mmol). The reaction mixture was warmed to 60° C. and stirred for 24 h, after which time the mixture was cooled to room temperature, concentrated and purified over a column of silica gel (100 g) eluting with 50% EtOAc in heptane to provide (2R,4R)-2-allyl 1-tert-butyl 4-(allyloxy)pyrrolidine-1,2-dicarboxylate (4.2 g, 13.49 mmol, 62%), and (2R,4R)-4-(allyloxy)-1-(tert-butoxycarbonyl)pyrrolidine-2-carboxylic acid (1.8 g, 6.63 mmol, 31%) (MW_{Calc}+Na=334.17; MW_{Obs}=334.16) after combining the desired fractions, concentration, and drying under vacuum.

To a stirred solution of 1 M LiAlH₄ in THF (20.23 mL, 20.233 mmol) in THF (42.0 mL) at 0° C. was added a solution of (2R,4R)-2-allyl 1-tert-butyl 4-(allyloxy)pyrrolidine-1,2-dicarboxylate (4.2 g, 13.489 mmol) in THF (50 mL) dropwise over 10 min. The reaction mixture was for 1 h at 0° C., after which time the completed reaction was carefully quenched with sat Na₂SO₄ (50 mL), stirred for 30 min at room temperature, and filtered through a pad of Celite (10 g) eluting with EtOAc (2×30 mL ea). The combined filtrates were concentrated to dryness to provide the desired crude alcohol.

To a stirred solution of the above alcohol in DCM (84 mL) at room temperature were added imidazole (2.75 g, 40.466 mmol) and TBS-CI (3.05 g, 20.233 mmol). The reaction mixture was stirred for 2 h followed by the addition of water (20 mL), and the resultant mixture was extracted with EtOAc (3×40 mL ea). The combined organic layers were dried over Na₂SO₄, filtered, and concentrated. The residue was purified over a Biotage Ultra SNAP column (50 g) eluting with a 10 CV gradient of 20 to 100% EtOAc in heptane to provide compound 266 (4.5 g, 12.11 mmol, 90%) (MW_{Calc}+Na=294.14; MW_{Obs}=294.20) after combining the desired fractions, concentration, and drying under vacuum.

To a stirred solution of (2R,4R)-tert-butyl 4-(allyloxy)-2-(((tert-butyldimethylsilyl)oxy)methyl)-pyrrolidine-1-carboxylate (264, 4.5 g, 12.11 mmol) in DCM (90 mL) and EtOH (90 mL) at −78° C. was bubbled through with ozone for 30 min. The completed reaction was purged with N₂ gas for 15 min, after which time sodium borohydride (0.687 g, 18.165 mmol) was added, and the mixture was allowed to warm to 0° C., stir for 3 h, warmed to room temperature, and stirred for 16 h. The mixture was quenched with sat NH₄Cl (20 mL), water (20 mL) was added followed by the mixture being extracted with EtOAc (3×50 mL ea). The combined organic layers were dried over Na₂SO₄, filtered, and concentrated. The residue was purified over a Biotage Ultra SNAP column (50 g) eluting with a 10 CV gradient of 0 to 100% EtOAc in heptane to provide (2R,4R)-tert-butyl 2-(((tert-butyldimethylsilyl)oxy)methyl)-4-(2-hydroxyethoxy)pyrrolidine-1-carboxylate (3.5 g, 9.32 mmol, 77%). after combining the desired fractions, concentration, and drying under vacuum.

To a stirred solution of (2R,4R)-tert-butyl 2-(((tert-butyldimethylsilyl)oxy)methyl)-4-(2-hydroxyethoxy)-pyrrolidine-1-carboxylate (2.0 g, 5.33 mmol) and methyl 4-hydroxy-3,5-dimethylbenzoate (1.15 g, 6.39 mmol) in THF (30.0 mL) at 0° C. were added triphenylphosphine (2.095 g, 7.988 mmol) and DIAD (1.553 mL, 7.988 mmol). The reaction mixture was warmed to room temperature, and stirred for 6 h, after which time it was concentrated, and purified over a Biotage Ultra SNAP column (25 g) eluting with a 10 CV gradient of 0 to 50% EtOAc in heptane to provide compound 267 (2.60 g, 4.83 mmol, 91%) after combining the desired fractions, concentration, and drying under vacuum.

To a stirred solution of (2R,4R)-tert-butyl 2-(((tert-butyldimethylsilyl)oxy)methyl)-4-(2-(4-(methoxycarbonyl)-2,6-dimethylphenoxy)ethoxy)pyrrolidine-1-carboxylate (267, 1.00 g, 1.86 mmol) in DCM (20 mL) at room temperature was added TFA (1.433 mL, 18.596 mmol). The reaction mixture was stirred for 0.5 h, after which time the mixture was concentrated, and azeotroped to dry with toluene (2×20 mL ea). The residue was dissolved with stirring in MeCN (15 mL) at room temperature followed by the addition of tetrahydro-2H-pyran-3-carboxylic acid (363 mg, 2.789 mmol), HOBT (0.285 g, 1.86 mmol), EDC (0.535 g, 2.789 mmol), and triethylamine (0.778 mL, 5.579 mmol). The final reaction mixture was stirred for 1.5 h, after which time it was diluted with EtOAc (20 mL) and washed with water (15 mL) and brine (15 mL). The organic layer was dried over Na₂SO₄, filtered, and concentrated. The residue was purified over a Biotage Ultra SNAP column (10 g) eluting with a 10 CV gradient of 40 to 100% EtOAc in heptane to provide 4-(2-(((3R,5R)-5-(hydroxymethyl)-1-(tetrahydro-2H-pyran-3-carbonyl)pyrrolidin-3-yl)oxy)ethoxy)-3,5-dimethylbenzoate (0.4 g, 0.918 mmol, 49%) after combining the desired fractions, concentration, and drying under vacuum.

To a stirred solution of methyl 4-(2-(((3R,5R)-5-(hydroxymethyl)-1-(tetrahydro-2H-pyran-3-carbonyl)pyrrolidin-3-yl)oxy)ethoxy)-3,5-dimethylbenzoate (0.41 g, 0.941 mmol) in THF (8.20 mL) at room temperature under a N₂ atmosphere were added allyl methyl carbonate (0.212 ml, 1.883 mmol) and Pd(Ph₃P)₄ (0.109 g, 0.094 mmol). The reaction mixture was warmed at 60° C. and stirred for 24 h, after which time it was concentrated to dry, and purified directly over a Biotage Ultra SNAP column (10 g) eluting with a 10 CV gradient of 0 to 100% EtOAc in heptane to provide methyl 4-(2-(((3R,5R)-5-((allyloxy)methyl)-1-(tetrahydro-2H-pyran-3-carbonyl)pyrrolidin-3-yl)oxy)ethoxy)-3,5-dimethylbenzoate (0.4 g, 0.841 mmol, 89%) (MW_{Calc}+Na=498.26; MW_{Obs}=498.19) after combining the desired fractions, concentration, and drying under vacuum.

To a stirred solution of methyl 4-(2-(((3R,5R)-5-((allyloxy)methyl)-1-(tetrahydro-2H-pyran-3-carbonyl)pyrrolidin-3-yl)oxy)ethoxy)-3,5-dimethylbenzoate (0.4 g, 0.841 mmol) in THF (5 mL) and MeOH (5 mL) at room temperature was added 1 N NaOH (4.21 mL, 4.21 mmol). The reaction mixture was stirred for 16 h, after which time it was acidified with 1 N HCl (4.5 mL) followed by the addition of brine (1 mL) and extracted with EtOAc (3×20 mL ea). The combined organic layers were dried over Na₂SO₄, filtered, and concentrated. The residue was purified over a Biotage Ultra SNAP column (25 g) eluting with a 10 CV gradient of 40 to 100% EtOAc in heptane, and then 5 CV of 10% MeOH in EtOAc to provide compound 268 (0.24 g, 0.520 mmol, 62%) (MW_{Calc}+Na=484.24; MW_{Obs}=484.15) after combining the desired fractions, concentration, and drying under vacuum.

To a stirred solution of (2R,4S,5R,6R)-methyl 2-allyl-6-((1S,2S)-3-azido-1,2-dihydroxypropyl)-5-((tert-butoxycarbonyl)amino)-4-hydroxytetrahydro-2H-pyran-2-carboxylate (13, 100 mg, 0.232 mmol) in THF (1.80 mL) and water (0.17 mL) at 0° C. was added 1N trimethylphosphine (0.17 mL, 0.17 mmol). The reaction mixture was warmed to room temperature, stirred for 16 h, then concentrated, and azeotroped to dry with toluene (2×10 mL ea). The resultant crude amine was dissolved with stirring in MeCN (1.20 mL) at room temperature followed by the addition of 4-(2-(((3R,5R)-5-((allyloxy)methyl)-1-(tetrahydro-2H-pyran-3-carbonyl)pyrrolidin-3-yl)oxy)ethoxy)-3,5-dimethylbenzoic acid (268, 161 mg, 0.348 mmol), HOBT (35.6 mg, 0.232 mmol), EDC (44.5 mg, 0.232 mmol), and triethylamine (32.4 μl, 0.232 mmol). The reaction mixture was stirred for 6 h, after which time it concentrated, then diluted with stirring in DCM (1 mL) at room temperature, followed by Et₃N (0.5 mL), DMAP (cat.) and Ac₂O (219 μl, 2.323 mmol). The final reaction mixture was stirred for 16 h, concentrated and then purified directly over a Biotage Ultra SNAP column (10 g) eluting with a 10 CV gradient of 40 to 100% EtOAc in heptane, then 5 CVs 10% MeOH in EtOAc. The desired fractions were combined, concentrated to dry, and then dissolved in EtOAc (20 mL). The organic solution was washed with 0.2 N NaOH (5 mL) and sat. NaHCO₃ (5 mL). The combined aqueous layer was extracted with EtOAc (5 mL), and the combined organic layers were dried over Na₂SO₄, filtered and concentrated to dry under vacuum to provide compound 269 (0.16 g, 0.164 mmol, 71%) (MW_{Calc}+H=974.48; MW_{Obs}=974.48).

To a stirred solution of (1R,2R)-1-((2R,3R,4S,6R)-4-acetoxy-6-allyl-3-((tert-butoxycarbonyl)amino)-6-(methoxycarbonyl)tetrahydro-2H-pyran-2-yl)-3-(4-(2-(((3R,5R)-5-((allyloxy)methyl)-1-(tetrahydro-2H-pyran-3-carbonyl)pyrrolidin-3-yl)oxy)ethoxy)-3,5-dimethylbenzamido)propane-1,2-diyl diacetate (269, 0.16 g, 0.164 mmol) in toluene (160 mL) at room temperature was added quinone (5.39 μL, 0.066 mmol), and Hoveyda-Grubbs Catalyst 2nd Generation (0.026 g, 0.041 mmol), The reaction mixture was warmed to 90° C. and stirred for 24 h, after which time it was cooled to room temperature and diluted with DMSO (0.2 mL). The mixture was concentrated to dry and purified over a Biotage Ultra SNAP column (25 g) eluting with a 10 CV gradient of 20 to 100% EtOAc in heptane, and then a 10 CV gradient of 0 to 10% MeOH in EtOAc to provide compound 270 (0.10 g, 0.104 mmol, 64%) (MW_{Calc}+H=946.45; MW_{Obs}=946.55) after combining the desired fractions, concentration, and drying under vacuum.

To a stirred solution of the protected macrocycle (270, 0.1 g, 0.106 mmol) in THF (4.00 mL) and methanol (4.00 mL) at room temperature was added 1 N NaOH (3.17 ml, 3.17 mmol). The reaction was stirred for 48 h, after which time it was acidified with 1 N HCl (1 mL), diluted with brine (1 mL), and extracted with EtOAc (3×5 mL ea). The combined organic layers were dried over Na₂SO₄, filtered, and concentrated. The residue was dissolved with stirring in DCM (3.4 mL) at room temperature followed by the addition of TFA (0.500 ml, 6.49 mmol). The mixture was stirred for 30 min, after which time the mixture was concentrated and azeotroped to dry with toluene (3×5 mL ea). The crude amine was dissolved in THF (2.50 mL), and then divided into four equal but separate portions.

To ¼^{th} of the resultant crude amine in THF at room temperature was added 10% aqueous sodium bicarbonate (0.222 g, 0.228 mmol) followed by acetic anhydride (0.020 ml, 0.211 mmol). The final reaction mixture was stirred for 15 min, after which time conc HCL was slowly added until obtained a pH<2. The resultant mixture was purified directly by HPLC to provide A-357 (11.0 mg, 0.015 mmol, 57%) (MW_{Calc}+H=748.36; MW_{Obs}=748.33) after combining the desired fractions, concentration, and drying under vacuum.

To a stirred solution of the protected macrocycle (270, 20 mg, 0.025 mmol) in DCM (1 mL) at room temperature was added TFA (19.12 μl, 0.248 mmol). The mixture was stirred for 30 min, after which time the mixture was concentrated and azeotroped to dry with toluene (2×5 mL ea) to provide the crude amine.

To a stirred solution of 2-(4-cyclohexyl-1H-1,2,3-triazol-1-yl)acetic acid (10.39 mg, 0.05 mmol) in MeCN (0.2 mL) at room temperature was added HOBT (7.60 mg, 0.05 mmol), EDC (9.51 mg, 0.05 mmol), and triethylamine (10.38 μl, 0.074 mmol). The mixture was stirred for 1 h, after which time a solution of the above crude amine in MeCN (200 μl, 3.829 mmol) was added followed by stirring for 4 h. The completed reaction was diluted with 1N HCl (0.20 mL) and MeOH (1 mL) and purified by HPLC to provide A-358 (10.0 mg, 0.011 mmol, 45%) (MW_{Calc}+H=897.46; MW_{Obs}=897.46) after combining the desired fractions, concentration, and drying under vacuum.

### Preparation of A-359

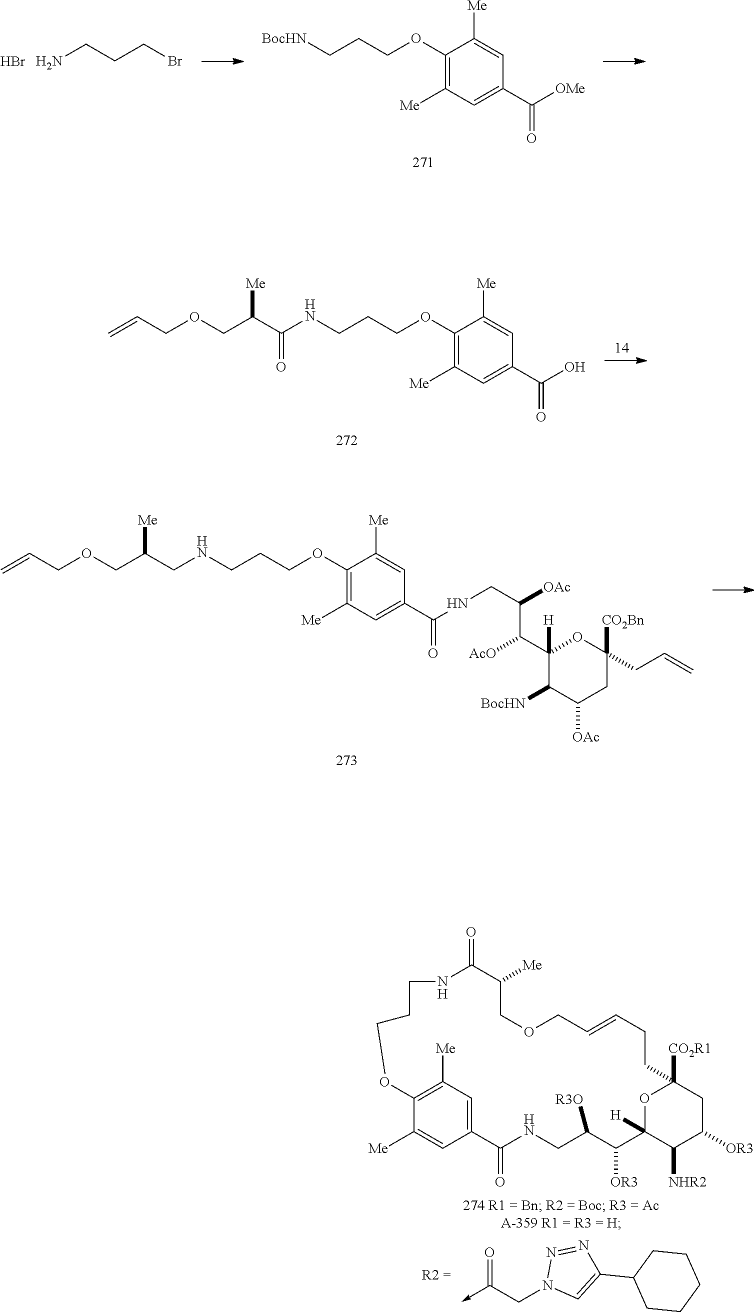

To a stirred solution of di-tert-butyl dicarbonate (7.48 g, 34.26 mmol) in DCM (200 mL) at 0° C. was added 3-bromopropan-1-amine hydrobromide (7.50 g, 34.26 mmol) followed by the dropwise addition of triethylamine (4.78 mL, 34.26 mmol) maintaining the temperature below 5° C. The resultant reaction mixture was warm to room temperature and stirred for 16 h. The completed reaction mixture was diluted with DCM (100 mL), washed with sat aq NH₄Cl (2×100 mL), sat aq NaHCO₃ (2×100 mL), and brine (2×50 mL). The organic layer was dried over Na₂SO₄, filtered, and concentrated, and dried under vacuum to provide tert-butyl (3-bromopropyl)carbamate (8.00 g, 33.60 mmol, 98%) (MW_{Calc}+Na=262.03; MW_{Obs}=262.02).

To a stirred solution of 4-hydroxy-3,5-dimethylbenzoic acid (0.5 g, 3.009 mmol) in DMF (10.0 mL) at room temperature were added potassium carbonate (1.248 g, 9.027 mmol) and tert-butyl (3-bromopropyl)carbamate (2.149 g, 9.027 mmol). The reaction mixture was warmed to 110° C. and stirred for 2 hours, after which time the incomplete reaction was cooled to room temperature followed by the addition of potassium carbonate (1.248 g, 9.027 mmol), and tert-butyl (3-bromopropyl)carbamate (2.149 g, 9.027 mmol). The reaction mixture was warmed again to 100° C., stirred for 2 h, then cooled to room temperature and diluted with water (50 mL). The resultant mixture was extracted with EtOAc (3×50 mL ea), and the combined organic layers were washed with water (2×25 mL), and brine (25 mL). The organic layer was dried over Na₂SO₄, filtered, and concentrated, and then purified over a Biotage Ultra SNAP column (25 g) eluting with a 10 CV gradient of 0 to 25% EtOAc in heptane to provide 3-((tert-butoxycarbonyl)amino)propyl 4-(3-((tert-butoxycarbonyl)amino)propoxy)-3,5-dimethylbenzoate (540 mg, 1.124 mmol, 37%) as an oil after combining the desired fractions, concentration, and drying under vacuum. The product was dissolved with stirring in MeOH (5 mL) and THF (5 mL) at room temperature followed by the addition of and 1N NaOH (10 mL, 10.0 mmol). The reaction was stirred for 5 h, after which time it was acidified with 1 N HCl to obtain a pH<2, and then extracted with EtOAc (3×10 mL ea). The combined organic layers were concentrated to dry, dissolved with stirring in toluene (5.0 mL) and methanol (0.5 mL) followed by the addition of 2 M TMS-diazomethane in methanol (1.504 ml, 3.009 mmol). The final reaction mixture was stirred for 20 min, concentrated to dry, and purified over a Biotage Ultra SNAP column (10 g) eluting with a 10 CV gradient of 0 to 25% EtOAc in heptane to provide compound 271 (316 mg, 0.937 mmol, 31%) (MW_{Calc}+Na=360.19; MW_{Obs}=360.26) as a crystalline solid after combining the desired fractions, concentration, and drying under vacuum.

To a stirred solution of methyl 4-(3-((tert-butoxycarbonyl)amino)propoxy)-3,5-dimethylbenzoate (271, 206 mg, 0.611 mmol) in DCM (1.44 mL) at room temperature was added TFA (1.44 mL, 18.717 mmol). The reaction mixture was stirred for 10 min, after which time it was concentrated, and azeotroped to dry with toluene (2×20 mL ea). The crude residue was dissolved with stirring in MeCN (5 mL) at room temperature followed by the addition of (R)-3-(allyloxy)-2-methylpropanoic acid (132 mg, 0.916 mmol), Et3N (340 μl, 2.442 mmol), and HATU (406 mg, 1.068 mmol). The reaction mixture was stirred for 45 min, after which time it was diluted with water (5 mL) and extracted with EtOAc (2×5 mL ea). The organic layers were washed with water (5 mL) and brine (5 mL), dried over Na₂SO₄, filtered, and concentrated, and then purified over a Biotage Ultra SNAP column (10 g) eluting with a 10 CV gradient of 0 to 50% EtOAc in heptane to provide (R)-methyl 4-(3-(3-(allyloxy)-2-methylpropanamido)propoxy)-3,5-dimethylbenzoate (222 mg, 0.611 mmol, 100%) (MW_{Calc}+Na=386.20; MW_{Obs}=386.28) as a white solid after combining the desired fractions, concentration, and drying under vacuum.

To a stirred solution of (R)-methyl 4-(3-(3-(allyloxy)-2-methylpropanamido)propoxy)-3,5-dimethylbenzoate (297 mg, 0.817 mmol) in THF (2.08 mL) and MeOH (2.08 mL) at room temperature was added 1 N sodium hydroxide (2.45 mL, 2.45 mmol). The reaction mixture was stirred for 3 h, after which time the mixture was acidified with 1 N HCl to obtain a pH<2. The mixture was extracted with EtOAc (4×5 mL ea), and the combined organic layers were washed with brine (5 mL), dried over Na₂SO₄, filtered, concentrated, and dried under vacuum to provide compound 272 (231 mg, 0.661 mmol, 81%) (MW_{Calc}+Na=372.19; MW_{Obs}=372.32) as a white crystalline solid, which was used in the next reaction without further purification.

To a stirred solution of (2R,4S,5R,6R)-methyl 2-allyl-6-((1R,2R)-3-amino-1,2-dihydroxypropyl)-5-((tert-butoxycarbonyl)amino)-4-hydroxytetrahydro-2H-pyran-2-carboxylate (14, 90 mg, 0.178 mmol) and (R)-4-(3-(3-(allyloxy)-2-methylpropanamido)propoxy)-3,5-dimethylbenzoic acid (272, 78 mg, 0.223 mmol) in MeCN (2 mL) at room temperature were added triethylamine (74.4 μl, 0.534 mmol), HOBT (13.63 mg, 0.089 mmol), and then EDC (51.2 mg, 0.267 mmol). The reaction mixture was stirred for 6 h, after which time the mixture was diluted with EtOAc (5 mL) and washed with water (2 mL) and brine (2 mL). The organic layer was dried over Na₂SO₄, filtered, concentrated, and purified over a Biotage Ultra SNAP column (4 g) eluting with a 10 CV gradient of 0 to 100% EtOAc in heptane to provide the desired amide intermediate (112 mg, 0.152 mmol, 85%) as a transparent film after combining the desired fractions, concentration, and drying under vacuum. The intermediate was used in the next reaction.

To a stirred solution of (2R,4S,5R,6R)-methyl 2-allyl-6-((1R,2R)-3-(4-(3-((R)-3-(allyloxy)-2-methylpropanamido)propoxy)-3,5-dimethylbenzamido)-1,2-dihydroxypropyl)-5-((tert-butoxycarbonyl)amino)-4-hydroxytetrahydro-2H-pyran-2-carboxylate (112 mg, 0.152 mmol) in methanol (1.0 mL) at room temperature was added 1 N sodium hydroxide (0.528 mL, 0.528 mmol). The reaction mixture was stirred for 24 h, after which time it was acidified with 1N HCL (0.7 mL), and then extracted with EtOAc (5×5 mL ea). The combined organic layers were washed with brine (5 mL), dried over Na₂SO₄, filtered, and concentrated to dry. The crude acid was dissolved with stirring in DMF (1.2 mL) at room temperature followed by the addition of potassium carbonate (73.8 mg, 0.534 mmol) and benzyl bromide (63.5 μL, 0.534 mmol). The reaction mixture was stirred for 2 h, after which time the mixture was diluted with water (5 mL) and extracted with EtOAc (2×5 mL). The combined organic layers were washed with water (2 mL) and brine (2 mL), dried over Na₂SO₄, filtered, and concentrated to dry. The crude oil was dissolved with stirring in pyridine (0.3 mL, 3.709 mmol) at room temperature followed by the addition of acetic anhydride (0.3 mL, 3.18 mmol). The final reaction mixture was stirred for 14 h, after which time it was diluted with EtOAc (20 mL), and the mixture was washed with NH₄Cl (5 mL), 1N HCl (5 mL), and brine (5 mL). The organic layer was dried over Na₂SO₄, filtered, concentrated, and purified over a Biotage Ultra SNAP column (4 g) eluting with a 10 CV gradient of 0 to 100% EtOAc in heptane to provide compound 273 (92 mg, 0.098 mmol, 55%) (MW_{Calc}+H=938.46; MW_{Obs}=938.44) as a film after combining the desired fractions, concentration, and drying under vacuum.

To a stirred solution of (1R,2R)-1-((2R,3R,4S,6R)-4-acetoxy-6-allyl-6-((benzyloxy)carbonyl)-3-((tert-butoxycarbonyl)amino)tetrahydro-2H-pyran-2-yl)-3-(4-(3-((R)-3-(allyloxy)-2-methylpropanamido)-propoxy)-3,5-dimethylbenzamido)propane-1,2-diyl diacetate (273, 0.090 g, 0.096 mmol) in toluene (63.0 mL) at room temperature was added 1,4-benzoquinone (5.0 mg, 0.046 mmol) followed by Hoveyda-Grubbs Catalyst 2nd Generation (6.0 mg, 0.0096 mmol). The reaction mixture was warmed to 80° C. and stirred for 1 h. The completed reaction was diluted with EtOAc (1 mL) and MeOH (1 mL) followed by concentration to dry. The residue was purified on 3 prep TLC silica gel plates eluting with 100% EtOAc (4×) to provide compound 274 (41 mg, 0.045 mmol, 47%) after scraping off the desired fraction, eluting the product off the silica gel with 10% MeOH in EtOAc (4×5 mL ea) via filtration, concentration of the filtrate, and drying under vacuum.

To a stirred solution of the fully protected macrocycle (274, 37 mg, 0.041 mmol) in DCM (260 μL) at room temperature was added TFA (259 μL, 3.362 mmol). The reaction mixture was stirred for 7 min, after which time it was concentrated, azeotroped to dry with toluene (2×5 mL ea), and placed under vacuum for 30 min. The crude amine was dissolved with stirring in MeCN (0.8 mL) at room temperature followed by the addition of 2-(4-cyclohexyl-1H-1,2,3-triazol-1-yl)acetic acid (12.76 mg, 0.061 mmol), triethylamine (22.67 μl, 0.163 mmol), and HATU (30.9 mg, 0.081 mmol). The reaction mixture was stirred for 1 h, after which time the mixture was diluted with water (2 mL) and extracted with EtOAc (3×2 mL ea). The combined organic layers were washed with water (2 mL) and brine (2 mL), followed by drying over. The resulting organic layer was Na₂SO₄, filtered, and concentrated to provide the crude amide (38 mg, 0.038 mmol, 93%) as an oil.

To a stirred solution of the crude amide from above (17 mg, 0.017 mmol) in ethanol (1.0 mL) and EtOAc (1.0 mL) at room temperature and previously purged with a N₂ atmosphere (3×), was added 10% Pd—C(20 mg, 0.188 mmol) followed by purging the solution with a H₂ atmosphere (3×) and stirring the mixture under a H₂ atmosphere under balloon pressure for 20 h. The completed reaction was purged with a N₂ atmosphere (4×) followed by the addition of Celite (0.2 g), filtration over a pad of Celite (0.2 g) and eluting with EtOAc (4×2 mL ea). The combined filtrates were concentrated, azeotroped to dry with MeOH (2×2 mL ea). The resulting residue was dissolved in methanol (0.6 mL) followed by the addition of 1N sodium hydroxide (0.119 mL, 0.119 mmol). The final reaction mixture was stirred for 30 min, after which time it was acidified with 1N HCl (0.2 mL) and purified directly by HPLC to provide A-359 (6.5 mg, 0.008 mmol, 49%) (MW_{Calc}+H=787.42; MW_{Obs}=787.52) after combining the desired fractions, concentration, and drying under vacuum.

Preparation of A-360 and other C1-Acid Replacement Analogs of A-001

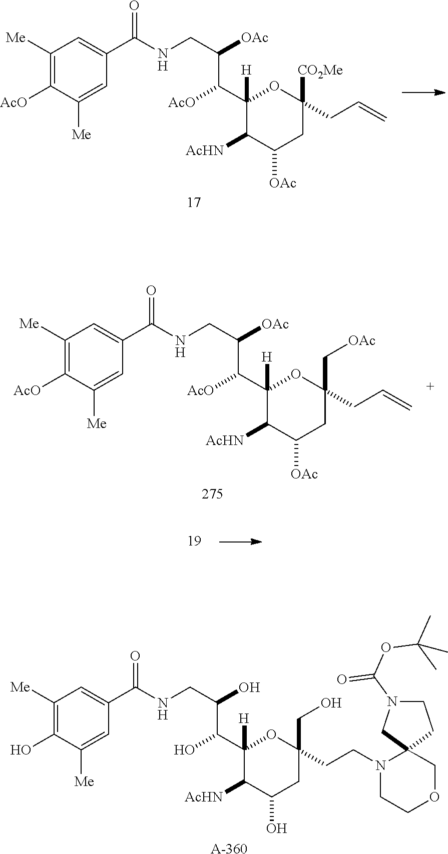

To a stirred solution of (1R,2R)-1-((2R,3R,4S,6R)-3-acetamido-4-acetoxy-6-allyl-6-(methoxycarbonyl)-tetrahydro-2H-pyran-2-yl)-3-(4-acetoxy-3,5-dimethylbenzamido)propane-1,2-diyl diacetate (17, 200 mg, 0.302 mmol) in methanol (4.50 mL) at room temperature was added 1 N NaOH (4.53 mL, 4.53 mmol). The reaction mixture was stirred for 16 h, after which time Dowex 50W×4 hydrogen form (3.4 g) was added rendering mixture acidic, followed by filtering, and eluting the resin with MeOH (3×5 mL ea). The combined filtrate was concentrated, and azeotroped to dry with MeCN (2×10 mL ea). The resulting residue was dissolve with stirring in pyridine (1.95 mL) at room temperature followed by the addition of DMAP (36.9 mg, 0.302 mmol) and Ac20 (368 μl, 15.09 mmol). The completed reaction was diluted with water (10 mL) and EtOAc (20 mL). The layers were separated, and the aqueous layer was extracted with EtOAc (1×10 mL). The combined organic layers were washed with brine (1×10 mL), dried over Na₂SO₄, filtered, concentrated, and purified over a Biotage Ultra SNAP column (10 g) eluting with 1 CV 5% EtOAc in heptane, a 10 CV gradient of 5 to 100% EtOAc in heptane, 1 CV EtOAc, a 5 CV gradient of 0 to 40% MeOH in EtOAc, and 1 CV of 40% MeOH in EtOAc to provide the desired acid intermediate (139 mg, 0.214 mmol, 71%) (MW_{Calc}+H=649.25; MW_{Obs}=649.29) after combining the desired fractions, concentration, and drying under vacuum. reaction.

To a stirred solution of (2R,4S,5R,6R)-5-acetamido-4-acetoxy-2-allyl-6-((1R,2R)-1,2-diacetoxy-3-(4-acetoxy-3,5-dimethylbenzamido)propyl)tetrahydro-2H-pyran-2-carboxylic acid (42.8 mg, 0.066 mmol) suspension in THF (0.50 mL) at room temperature was added n-methylmorpholine (36.3 μl, 0.33 mmol) followed by addition of benzyl chloroformate (37.7 μl, 0.264 mmol). The mixture was stirred for 1 h, after which time it was cooled 0° C., and then added sodium borohydride (24.96 mg, 0.66 mmol) followed by addition of methanol (200 μl, 4.944 mmol) while maintaining the temperature at 0° C. The reaction mixture was stirred for 30 min, after which time it was quenched with slowly 2 M citric acid (2 mL) and diluted with EtOAc (5 mL). The separated organic layer was washed with water (2 mL) and a 1:1 solution of water in brine (2 mL), dried over Na₂SO₄, filtered, concentrated, and azeotroped to dry with MeCN (2×5 mL ea). The residue was dissolved with stirring in pyridine (213 μl, 2.639 mmol) at room temperature followed by the addition Ac₂O (249 μl, 2.639 mmol), and DMAP (8.06 mg, 0.066 mmol). The reaction mixture was stirred for 16 h, after which time it was diluted with water (5 mL) and EtOAc (5 mL). The layers were separated, and the aqueous layer was extracted with EtOAc (1×5 mL). The combined organic layers were washed with sat. NaHCO₃ (3 mL), a 1:1 solution of water in brine (2 mL), dried over Na₂SO₄, filtered, and concentrated. The residue was purified over a Biotage Ultra SNAP column (4 g) eluting with 1 CV 5% EtOAc in heptane, a 10 CV gradient of 5 to 100% EtOAc in heptane, 1 CV EtOAc, and a 5 CV gradient of 0 to 50% MeOH in EtOAc to provide compound 275 (31.6 mg, 0.047 mmol, 71%) (MW_{Calc}+H=677.28; MW_{Obs}=677.35) after combining the desired fractions, concentration, and drying under vacuum. The intermediate was used in the next reaction.

To a stirred solution of (1R,2R)-1-((2R,3R,4S,6R)-3-acetamido-4-acetoxy-6-(acetoxymethyl)-6-allyltetrahydro-2H-pyran-2-yl)-3-(4-acetoxy-3,5-dimethylbenzamido)propane-1,2-diyl diacetate (275, 31.6 mg, 0.047 mmol) in 1,4-dioxane (900 μL and water (300 μL) at room temperature were added 2,6-lutidine (10.88 μl, 0.093 mmol), sodium periodate (40.0 mg, 0.187 mmol) and osmium tetroxide (29.7 μl, 4.67 μmol). The reaction mixture was stirred for 3 h, after which time it was diluted with a mixture of 1:1 EtOAC in 15% aq Na₂S₂O₃ solution, the layers separated, and the aqueous layers was extracted with EtOAc (5 mL). The combined organic layers were washed with sat. NaHCO₃ (3 mL), a 1:1 solution of water in brine (2 mL), dried over Na₂SO₄, filtered, concentrated, and dried under vacuum to provide the crude aldehyde (31 mg, 0.046 mmol, 98%) (MW_{Calc}+H=679.26; MW_{Obs}=679.41).

To a stirred solution of crude (1R,2R)-1-((2R,3R,4S,6S)-3-acetamido-4-acetoxy-6-(acetoxymethyl)-6-(2-oxoethyl)tetrahydro-2H-pyran-2-yl)-3-(4-acetoxy-3,5-dimethylbenzamido)propane-1,2-diyl diacetate (6.8 mg, 0.010 mol) and (S)-tert-butyl 9-oxa-2,6-diazaspiro[4.5]decane-2-carboxylate (19, 4.86 mg, 0.02 mmol) in DCE (800 μL) at room temperature were added acetic acid (4.0 μL, 0.07 mmol) and activated molecular sieves 4A (80 mg). The suspension was stirred for 3 h, after which time sodium triacetoxyborohydride (13 mg, 0.061 mmol) was added. The reaction mixture was stirred for 12 h, after which time the mixture as quenched with sat. NaHCO₃ (3 mL) and diluted with DCM (5 mL). The mixture was filtered over a pad of Celite (2 g), eluted with EtOAc (2×2 mL ea), and the filtrate layers were separated. The organic layer was extracted with EtOAc (3 mL), and the combined organic layers were washed with a 1:1 solution of water in brine (2 mL), dried over Na₂SO₄, filtered, concentrated, and dried under vacuum. The crude residue was dissolved in MeOH (600 μL) and water (150 μL) at room temperature followed by the addition of 1 N NaOH (150 μl, 0.15 mmol). The final reaction mixture was stirred for 4.5 h, after which time it was acidified with 1 N HCl (150 μl, 0.15 mmol) to pH 5, adjusted the pH to 6-7 with 1 N NaOH (approx. 60 uL) and purified directly by HPLC to provide A-360 (5.8 mg, 0.008 mmol, 83%) (MW_{Calc}+H=695.38; MW_{Obs}=695.6) after combining the desired fractions, concentration, and drying under vacuum.

### Preparation of A-361

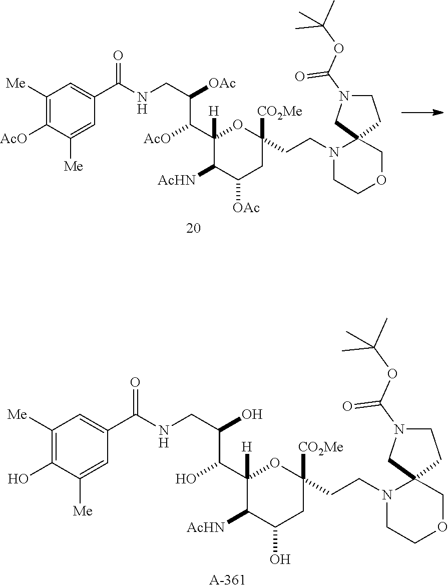

To a vial containing (1R,2R)-1-((2R,3R,4S,6R)-3-acetamido-4-acetoxy-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)-3-(4-acetoxy-3,5-dimethylbenzamido)propane-1,2-diyl diacetate (20, 20.3 mg, 0.023 mmol) was added 7 N ammonia (2 ml, 14.00 mmol) in methanol at room temperature. The reaction vessel was sealed and placed in microwave reactor at 120° C. for 1 h, after which time it was cooled to room temperature, concentrated to dry, diluted in MeOH (1 mL), and purified by HPLC to provide A-361(8.5 mg, 0.012 mmol, 51%) (MW_{Calc}+H=723.37; MW_{Obs}=723.54) after combining the desired fractions, concentration, and drying under vacuum. A-361 is also A-002.

### Preparation of A-362 and A-363

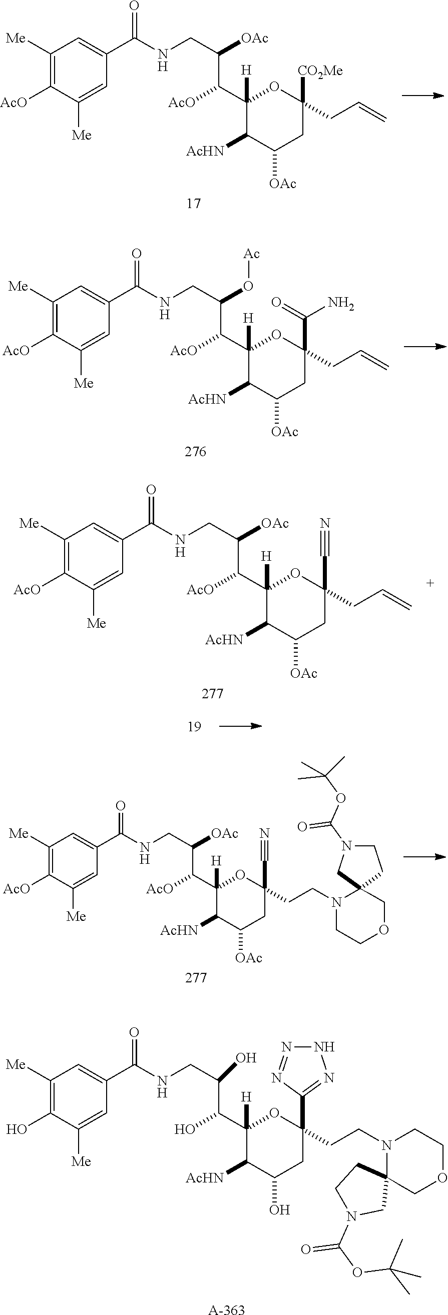

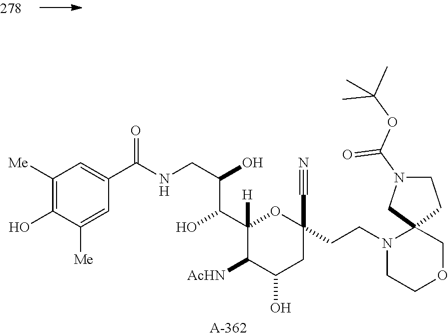

To a stirred solution of (1R,2R)-1-((2R,3R,4S,6R)-3-acetamido-4-acetoxy-6-allyl-6-(methoxycarbonyl)-tetrahydro-2H-pyran-2-yl)-3-(4-acetoxy-3,5-dimethylbenzamido)propane-1,2-diyl diacetate (17, 200 mg, 0.302 mmol) in methanol (4.50 mL) at room temperature was added 1 N NaOH (4.53 mL, 4.53 mmol). The reaction mixture was stirred for 16 h, after which time Dowex 50W×4 hydrogen form (3.4 g) was added rendering mixture acidic, followed by filtering, and eluting the resin with MeOH (3×5 mL ea). The combined filtrate was concentrated, and azeotroped to dry with MeCN (2×10 mL ea). The resulting residue was dissolve with stirring in pyridine (1.95 mL) at room temperature followed by the addition of DMAP (36.9 mg, 0.302 mmol) and Ac20 (368 μl, 15.09 mmol). The completed reaction was diluted with water (10 mL) and EtOAc (20 mL). The layers were separated, and the aqueous layer was extracted with EtOAc (1×10 mL). The combined organic layers were washed with brine (1×10 mL), dried over Na₂SO₄, filtered, concentrated, and purified over a Biotage Ultra SNAP column (10 g) eluting with 1 CV 5% EtOAc in heptane, a 10 CV gradient of 5 to 100% EtOAc in heptane, 1 CV EtOAc, a 5 CV gradient of 0 to 40% MeOH in EtOAc, and 1 CV of 40% MeOH in EtOAc to provide the desired acid intermediate (139 mg, 0.214 mmol, 71%) (MW_{Calc}+H=649.25; MW_{Obs}=649.29) after combining the desired fractions, concentration, and drying under vacuum. The intermediate was used in the next reaction.

To a stirred solution of (2R,4S,5R,6R)-5-acetamido-4-acetoxy-2-allyl-6-((1R,2R)-1,2-diacetoxy-3-(4-acetoxy-3,5-dimethylbenzamido)propyl)tetrahydro-2H-pyran-2-carboxylic acid (73.4 mg, 0.113 mmol) in acetonitrile (2.0 mL) at room temperature were added DMAP (8.4 mg, 0.069 mmol), triethylamine (79 μL, 0.566 mmol), 4-methoxybenzylamine (44.4 μL, 0.339 mmol) and HATU (129 mg, 0.339 mmol), The reaction mixture was stirred for 1 h, after which time it was diluted with water (5 mL) and EtOAc (5 mL), the layers were separated, and the aqueous layer was extracted with EtOAc (5 mL). The combined organic layers were washed with a 1:1 solution of water in brine (2 mL), dried over Na₂SO₄, filtered, concentrated, and dried under vacuum to provide the crude amide that was used directly in the next reaction. (MW_{Calc}+H=768.33; MW_{Obs}=768.54).

To a stirred solution of (1R,2R)-1-((2R,3R,4S,6R)-3-acetamido-4-acetoxy-6-allyl-6-((4-methoxybenzyl)carbamoyl)tetrahydro-2H-pyran-2-yl)-3-(4-acetoxy-3,5-dimethylbenzamido)-propane-1,2-diyl diacetate (87 mg, 0.113 mmol) in acetonitrile (4.0 mL) and water (2.0 mL) at room temperature was added CAN (124 mg, 0.227 mmol). The reaction mixture was stirred for 14 h, after which time CAN (571 mg, 1.042 mmol) was added. The resultant mixture was stirred for 2 h, after which time it was diluted with DCM (10 mL) and quenched with sat. NaHCO₃ (5 mL). The layers were separated, and the aqueous layer was extracted with DCM (1×10 mL). The combined organic layers were washed with brine (1×10 mL), dried over Na₂SO₄, filtered, concentrated, and purified over a Biotage Ultra SNAP column (4 g) eluting with 2 CV heptane, a 12 CV gradient of 0 to 100% EtOAc in heptane, 1 CV EtOAc, and a 5 CV gradient of 0 to 30% MeOH in EtOAc to provide compound 276 (72 mg, 0.092 mmol, 81%) (MW_{Calc}+Na=670.27; MW_{Obs}=670.47) after combining the desired fractions, concentration, and drying under vacuum. The intermediate was used in the next reaction.

To a stirred solution of (2R,4S,5R,6R)-5-acetamido-2-allyl-6-((1R,2R)-1,2-dihydroxy-3-(4-hydroxy-3,5-dimethylbenzamido)propyl)-4-hydroxytetrahydro-2H-pyran-2-carboxamide (276, 33 mg, 0.069 mmol) in THF (2.0 mL) at room temperature were added pyridine (111 μL, 1.376 mmol) and TFAA (97 μl, 0.688 mmol). The reaction mixture was stirred for 2 h, after which time the mixture was quenched with methanol (5.0 mL), triethylamine (480 μL, 3.441 mmol), and stirred for an additional 3 h. The completed reaction was concentrated, and azeotroped to dry with MeCN (3×10 mL ea). The resulting residue was dissolved with stirring in DCE (6.0 mL) at room temperature followed by the addition of DMAP (8.4 mg, 0.069 mmol), pyridine (835 μL, 10.32 mmol) and Ac₂O (974 μL, 10.32 mmol). The reaction mixture was stirred for 16 h, after which time the mixture was cooled to 0° C., diluted with water (10 mL) and EtOAc (10 mL), the layers were separated, and the aqueous layer was extracted with EtOAc (10 mL). The combined organic layers were washed with sat NaHCO₃ (3 mL), a 1:1 solution of water in brine (2 mL), dried over Na₂SO₄, filtered, and concentrated. The residue was purified over a Biotage Luknova column (4 g) eluting with 2 CV heptane, a 12 CV gradient of 0 to 100% EtOAc in heptane, 2 CV EtOAc, and a 5 CV gradient of 0 to 20% MeOH in EtOAc to provide compound 277 (35.4 mg, 0.056 mmol, 82%) (MW_{Calc}+H=630.26; MW_{Obs}=630.41) after combining the desired fractions, concentration, and drying under vacuum.

To a stirred solution of (1R,2R)-1-((2R,3R,4S,6R)-3-acetamido-4-acetoxy-6-allyl-6-cyanotetrahydro-2H-pyran-2-yl)-3-(4-acetoxy-3,5-dimethylbenzamido)propane-1,2-diyl diacetate (277, 38.4 mg, 0.061 mmol) in methanol (3 mL) and DCM (1 mL) at −78° C. was added ozone over a 15 min period after which time the reaction was rendered ozone free by bubbling N2 into the reaction for 10 min at −78° C. Dimethyl sulfide (0.1 ml, 1.36 mmol) added and the completed reaction was warmed to room temperature followed by diluting with EtOAc (5 mL) and water (2 mL). The layers were separated, and the organic layer was dried over Na₂SO₄, filtered and concentrated to dry. The residue along with (S)-tert-butyl 9-oxa-2,6-diazaspiro[4.5]decane-2-carboxylate (19, 29.6 mg, 0.122 mmol) were dissolve with stirring in DCE (3 mL) at room temperature followed by the addition acetic acid (24.4 μl, 0.427 mmol), and 4 Å molecular sieves (450 mg). The suspension was stirred for 2 h, after which time was added sodium triacetoxyborohydride (78 mg, 0.366 mmol), and the resultant mixture was stirred for 2 h. The complete reaction was diluted with DCM (5 mL) and quenched with sat. NaHCO₃ (5 mL). The layers were separated, and the aqueous layer was extracted with DCM (1×5 mL). The combined organic layers were washed with a 1:1 solution of water in brine (5 mL), dried over Na₂SO₄, filtered, concentrated, and purified over a Biotage Luknova column (4 g) eluting with 1 CV 5% EtOAc in heptane, a 6 CV gradient of 5 to 100% EtOAc in heptane, 1 CV EtOAc, and a 6 CV gradient of 0 to 60% MeOH in EtOAc to provide compound 278 (46.6 mg, 0.054 mmol, 89%) (MW_{Calc}+Na=880.41; MW_{Obs}=880.51) after combining the desired fractions, concentration, and drying under vacuum.

To a vial of (1R,2R)-1-((2R,3R,4S,6R)-3-acetamido-4-acetoxy-6-(2-((S)-2-(tert-butoxycarbonyl)-9-oxa-2,6-diazaspiro[4.5]decan-6-yl)ethyl)-6-cyanotetrahydro-2H-pyran-2-yl)-3-(4-acetoxy-3,5-dimethyl-benzamido)propane-1,2-diyl diacetate (278, 30 mg, 0.035 mmol) in THF (1.20 mL) at room temperature was added azidotrimethylsilane (371 μL, 2.797 mmol). The reaction mixture vessel was sealed and placed in a microwave reactor at 150° C. for 5 h, cooled to room temperature, added more azidotrimethylsilane (200 μL, 1.507 mmol), and then microwaved at 150° C. for 23 h. The completed reaction was cooled to room temperature, concentrated, and dissolved in methanol (1.2 mL) followed by 1 N NaOH (700 μl, 0.70 mmol). The resulting mixture was stirred for 16 h, after which time it was acidified with 1 N HCl (580 μl, 0.58 mmol) and purified directly by HPLC to provide A-363 (3.4 mg, 0.005 mmol, 13%) (MW_{Calc}+H=690.37; MW_{Obs}=690.57) after combining the desired fractions, concentration, and drying under vacuum.

To a stirred solution of (1R,2R)-1-((2R,3R,4S,6R)-3-acetamido-4-acetoxy-6-(2-((S)-2-(tert-butoxycarbonyl)-9-oxa-2,6-diazaspiro[4.5]decan-6-yl)ethyl)-6-cyanotetrahydro-2H-pyran-2-yl)-3-(4-acetoxy-3,5-dimethylbenzamido)propane-1,2-diyl diacetate (278, 8.1 mg, 9.4 μmol) in methanol (600 μl, 14.831 mmol) and water (200 μL, 11.102 mmol) at room temperature was added 1 N NaOH (142 μl, 0.142 mmol). The reaction mixture was stirred for 3.5 h, after which time it was neutralized with 1 N HCl (0.15 mL, 0.15 mmol), and then purified directly by HPLC to provide A-362 (2.6 mg, 0.004 mmol, 40%) (MW_{Calc}+H=733.38; MW_{Obs}=733.55) after combining the desired fractions, concentration, and drying under vacuum.

### Preparation of A-364

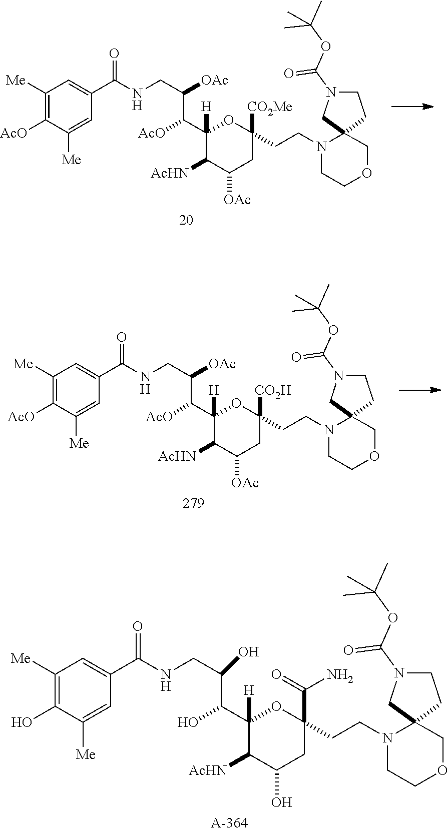

To a stirred solution of (1R,2R)-1-((2R,3R,4S,6R)-3-acetamido-4-acetoxy-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)-3-(4-acetoxy-3,5-dimethylbenzamido)propane-1,2-diyl diacetate (20, 373.2 mg, 0.419 mmol) in MeOH (6.28 mL) at room temperature was added 1N NaOH (4.19 mL, 4.189 mmol). The reaction mixture was stirred for 72 h, after which time the mixture was cooled to 0° C. followed by the portion wise addition of Dowex 50W×4 hydrogen form (3 g). The suspension was filtered, eluting the filtrate with MeOH (2×5 mL ea), and the combined filtrates were concentrated, and azeotroped to dry with MeCN (3×5 mL ea) to provide the fully deprotected acid (141 mg). The residue was dissolved with stirring in DCM (3.00 mL) at room temperature followed by the addition of DMAP (7 mg, 0.057 mmol), triethylamine (0.584 mL, 4.189 mmol), and Ac₂O (0.316 mL, 3.351 mmol). The reaction mixture was stirred for 3 h, after which time it was cooled 0° C., and slowly quenched with ethanol (0.50 mL). The mixture was stirred for 10 min, concentrated, azeotroped to dry with MeCN (2×5 mL ea), and the purified over a Biotage Luknova column (4 g) eluting with 2 CV 5% EtOAc in heptane, a 6 CV gradient of 5 to 100% EtOAc in heptane, 2 CV EtOAc, and a 8 CV gradient of 0 to 80% MeOH in EtOAc to provide compound 279 (70.0.080 mmol, 19%) (MW_{Calc}+H=877.40; MW_{Obs}=877.58) after combining the desired fractions, concentration, and drying under vacuum.

To a stirred solution of (2R,4S,5R,6R)-5-acetamido-4-acetoxy-2-(2-((S)-2-(tert-butoxycarbonyl)-9-oxa-2,6-diazaspiro[4.5]decan-6-yl)ethyl)-6-((1R,2R)-1,2-diacetoxy-3-(4-acetoxy-3,5-dimethylbenzamido)-propyl)tetrahydro-2H-pyran-2-carboxylic acid (279, 11.7 mg, 0.013 mmol) in DMF (300 μL) at room temperature were added DMAP (1.6 mg, 0.013 mmol), triethylamine (37.2 μL, 0.267 mmol) and HATU (50.7 mg, 0.133 mmol). The reaction mixture was stirred for 6 min, after which time ammonium chloride (10.71 mg, 0.20 mmol) was added followed by stirring for 16 h. The completed reaction was diluted with DCM (5 mL) and sat. NaHCO₃ (3 mL), the layers were separated and the aqueous layer was extracted with DCM (3 mL). The combined organic layers were washed with a 1:1 solution of water in brine (4 mL), dried over Na₂SO₄, filtered, and concentrated. The residue was dissolved with stirring in methanol (600 μL) and water (200 μL) at 0° C. followed by the addition of 1 N NaOH (107 μL, 0.107 mmol). The reaction mixture was stirred at 0° C. for 1 h, after which time the mixture was acidified with 1 N HCl (117 μL, 0.117 mmol) and directly purified by HPLC to provide A-364 (2.2 mg, 0.003 mmol, 23%) (MW_{Calc}+H=708.38; MW_{Obs}=708.6) after combining the desired fractions, concentration, and drying under vacuum.

### Preparation of A-365 to A-368

A-365 was prepared in a similar fashion to A-364 starting with compound 277 (42 mg, 0.048 mmol) condensed with methanesulfonamide (45.6 mg, 0.479 mmol) in DMF (1.0 mL) with diisopropylamine (0.10 mL, 0.573 mmol) and HATU (54.6 mg, 0.144 mmol) to provide A-365 (15.0 mg, 0.019 mmol, 40%) (MW_{Calc}+H=786.36; MW_{Obs}=786.65) after hydrolysis with 1 N NaOH of the —O-acetyl protecting groups, neutralization, HPLC purification, combining the desired fractions, concentration, and drying under vacuum.

A-366 was prepared in a similar fashion to A-364 starting with compound 277 (42 mg, 0.048 mmol) condensed with cyanimide (36 mg, 0.856 mmol) in DMF (1.0 mL) with diisopropylamine (0.10 mL, 0.573 mmol) and HATU (54.6 mg, 0.144 mmol) to provide A-366 (21.0 mg, 0.029 mmol, 60%) (MW_{Calc}+H=733.37; MW_{Obs}=733.63) after hydrolysis with 1 N NaOH of the —O-acetyl protecting groups, neutralization, HPLC purification, combining the desired fractions, concentration, and drying under vacuum.

A-367 was prepared in a similar fashion to A-364 starting with compound 277 (42 mg, 0.048 mmol) condensed with trifluoromethanesulfonamide (71.4 mg, 0.479 mmol) in DMF (1.0 mL) with diisopropylamine (0.10 mL, 0.573 mmol) and HATU (54.6 mg, 0.144 mmol) to provide A-367 (29.0 mg, 0.035 mmol, 73%) (MW_{Calc}+H=840.33; MW_{Obs}=840.63) after hydrolysis with 1 N NaOH of the —O-acetyl protecting groups, neutralization, HPLC purification, combining the desired fractions, concentration, and drying under vacuum.

A-368 was prepared in a similar fashion to A-364 starting with compound 277 (42 mg, 0.048 mmol) condensed with benzenesulfonamide (75 mg, 0.479 mmol) in DMF (1.0 mL) with diisopropylamine (0.10 mL, 0.573 mmol) and HATU (54.6 mg, 0.144 mmol) to provide A-368 (16.7 mg, 0.020 mmol, 42%) (MW_{Calc}+H=848.37; MW_{Obs}=848.68) after hydrolysis with 1 N NaOH of the —O-acetyl protecting groups, neutralization, HPLC purification, combining the desired fractions, concentration, and drying under vacuum.

### General Procedure for the Preparation of Multivalent and Conjugated Analogs of Neuraminic Acid Multivalent Analogs (Tri- and Divalent)

### Preparation of A-369

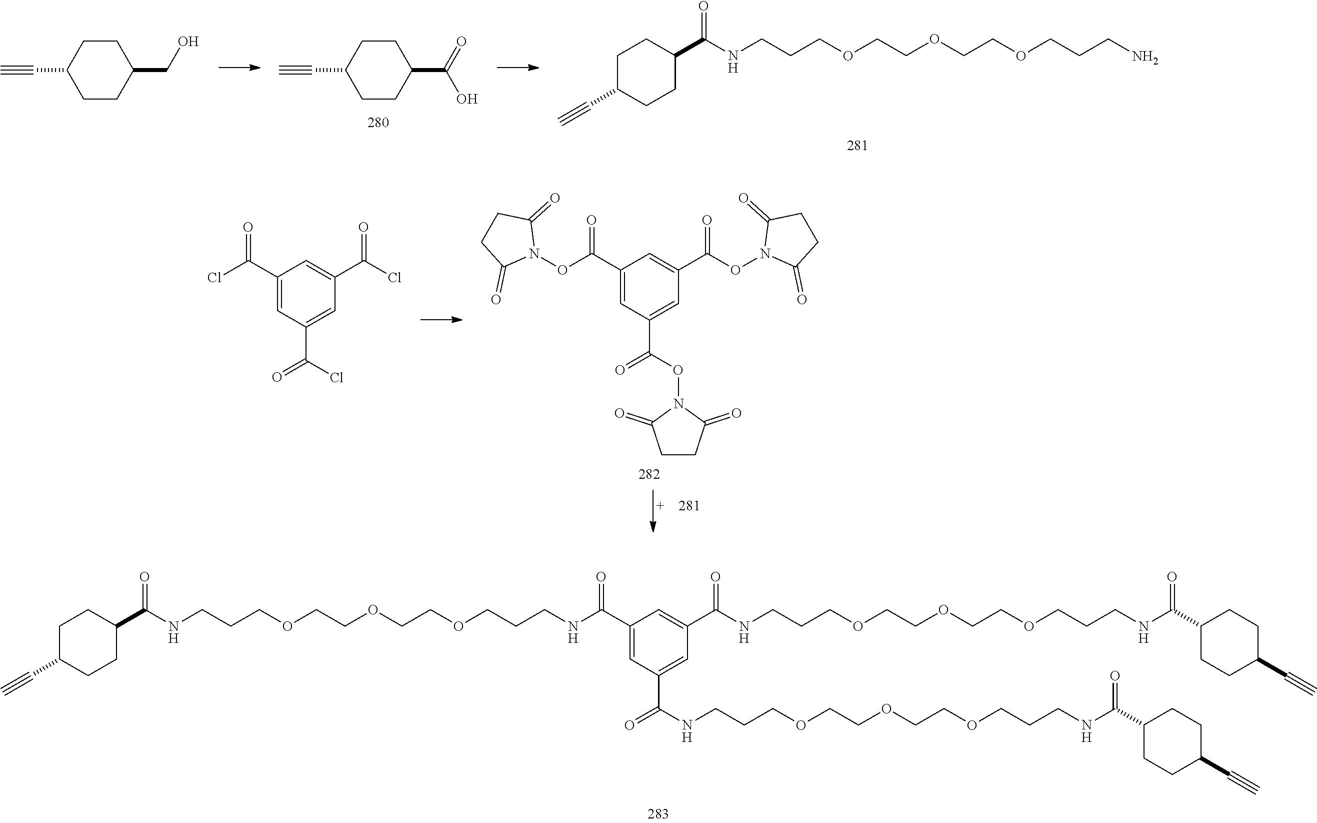

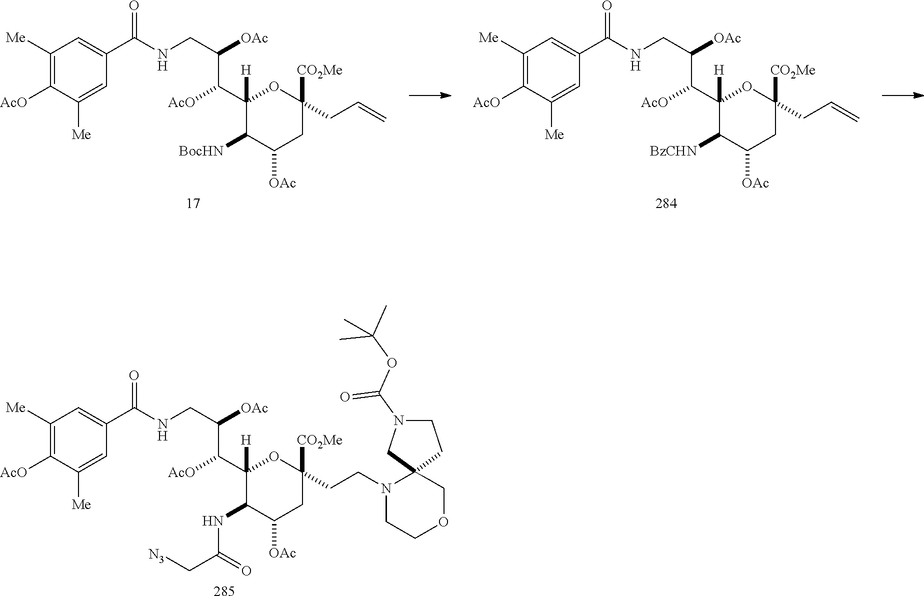

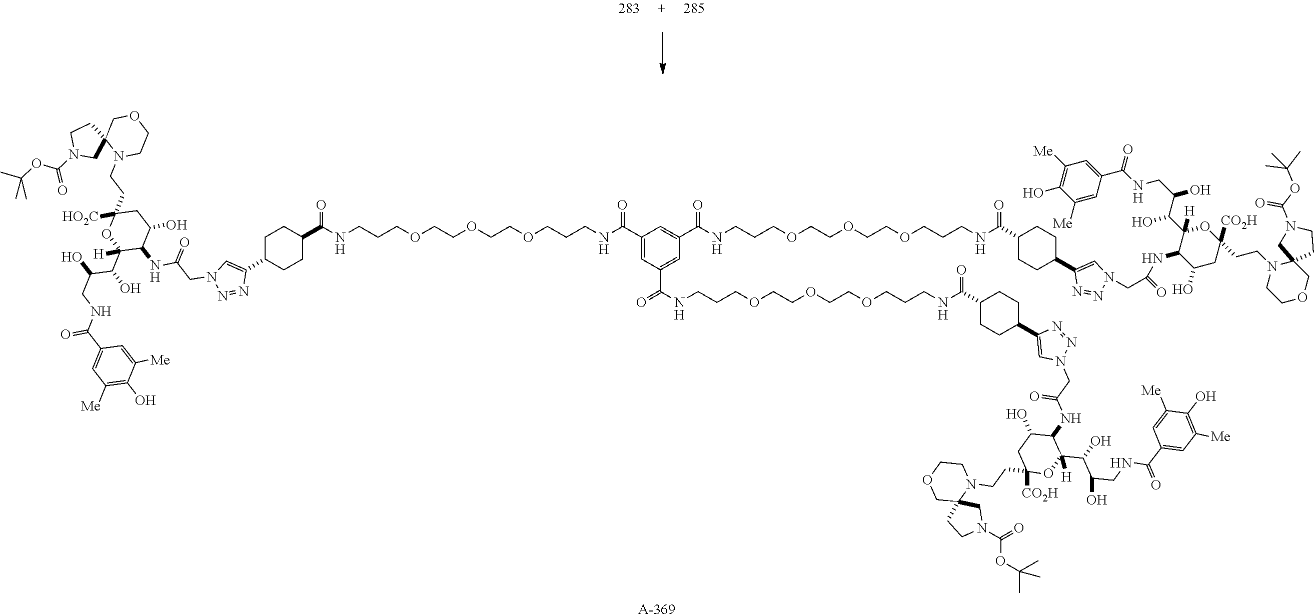

To a stirred solution of ((1R,4R)-4-ethynylcyclohexyl)methanol (1.00 g, 7.236 mmol) in acetone (20.0 ml, 272.387 mmol) at 0° C. was slowly added Jones' oxidation reagent (5.43 ml, 10.853 mmol). The reaction mixture was allowed to slowly warm to room temperature and stirred for 72 h. The completed reaction was quenched with 2-propanol (8.36 ml, 108.533 mmol) and stirred for 1 h. The mixture filtered through a pad of Celite (5 g), eluting with acetone (5 mL), and a 1:1 mixture of DCM/acetone (10 mL). The filtrate was concentrated to dryness, and the residue was dissolved in dry acetone (20 mL), dried over Na₂SO₄, filtered through a pad of Celite (5 g), eluting with acetone (3×5 mL). The filtrate was concentrated to dry to give an off-white solid, which was crystalized from a 1:1 ration of acetone in water (10 mL) to provide compound 280 (890 mg, 5.85 mmol, 81%) after collection of the solid, and drying under vacuum for 48 h.

To a stirred solution of tert-butyl (3-(2-(2-(3-aminopropoxy)ethoxy)ethoxy)propyl)carbamate (2.0 g, 6.242 mmol) and (1R,4R)-4-ethynylcyclohexanecarboxylic acid (280, 890 mg, 5.848 mmol) in DCM (20 mL) at room temperature were added HATU (2.89 g, 7.602 mmol) and Hunig's Base (2.229 mL, 12.763 mmol). The reaction was stirred for 2 h, after which time the mixture was concentrated, and purified directly over Biotage Ultra SNAP column (50 g) eluting with a 6 CV gradient of 70 to 100% EtOAc in heptane, and 3 CV EtOAc to provide the desired amide (266 mg, 0.05.85 mmol, 100%) (MW_{Calc}+H=455.40; MW_{Obs}=455.42) after combining the desired fractions, concentration, and drying under vacuum. The intermediate was used in the next reaction.

To a stirred solution of tert-butyl (1-((1R,4R)-4-ethynylcyclohexyl)-1-oxo-6,9,12-trioxa-2-azapentadecan-15-yl)carbamate (2.66 g, 5.851 mmol) in DCM (15 mL) at room temperature was added dropwise TFA (15 mL, 194.697 mmol). The reaction mixture was stirred for 20 min, after which time the mixture was concentrated, and azeotroped to dry with toluene (10 mL). The residue was purified directly over Biotage Ultra SNAP column (50 g) eluting with a 10 CV gradient of 5 to 15% MeOH in DCM, and 3 CV with 15% MeOH in DCM to provide compound 281 (2.07 g, 5.84 mmol, 100%) (MW_{Calc}+H=355.25; MW_{Obs}=355.34) after combining the desired fractions, concentration, and drying under vacuum. The trifluoroacetate salt was formed by the addition of 1 equivalent of trifluoroacetic acid in DCM followed by azeotroping to dry with toluene.

To a stirred solution of benzene-1,3,5-tricarbonyl trichloride (5.0 g, 18.834 mmol) in DCM (100 mL) at 0° C. was added 1-hydroxypyrrolidine-2,5-dione (7.59 g, 65.919 mmol) followed by dropwise addition of TEA (10.50 mL, 75.336 mmol) over 5 min. The reaction mixture was stirred at 0° C. for 1 h, warmed to room temperature, and stirred for an additional 48 h. The solid from the reaction mixture was filtered, washed with DCM (70 mL), and the resulting solid was suspended in DCM (200 mL), and stirred for 10 min. The solid was filtered and dried under vacuum for a minimum of 24 h to provide 282 (6.03 g, 12.03 mmol, 64%) (MW_{Calc}+Na=524.07; MW_{Obs}=524.23).

To a stirred solution of tris(2,5-dioxopyrrolidin-1-yl) benzene-1,3,5-tricarboxylate (282, 100 mg, 0.199 mmol) and (1R,4R)—N-(3-(2-(2-(3-aminopropoxy)ethoxy)ethoxy)propyl)-4-ethynylcyclohexane-1-carboxamide bis(2,2,2-trifluoroacetate) (281, 407 mg, 0.698 mmol) in DMF (3.0 mL) at room temperature was added TEA (0.278 mL, 1.995 mmol). The reaction mixture was stirred for 16 h, after which time it was concentrated, and purified directly over Biotage Ultra SNAP column (10 g) eluting with a 6 CV gradient of 2 to 7.5% MeOH in DCM, and 3 CV with 17.5% MeOH in DCM to provide compound 283 (213 mg, 0.175 mmol, 88% yield) (MW_{Calc}+H=1219.74; MW_{Obs}=1219.75) as a colorless oil after combining the desired fractions, concentration, and drying under vacuum. The intermediate was used in the next reaction.

To a stirred solution of (1R,2R)-3-(4-acetoxy-3,5-dimethylbenzamido)-1-((2R,3R,4S,6R)-4-acetoxy-6-allyl-3-((tert-butoxycarbonyl)amino)-6-(methoxycarbonyl)tetrahydro-2H-pyran-2-yl)propane-1,2-diyl diacetate (31, 360 mg, 0.499 mmol) at room temperature were added DCM (3 mL) and TFA (3 mL). The reaction mixture was stirred for 20 min, after which time the mixture was concentrated, and azeotroped to dry with toluene (3×10 mL ea). The resultant residue was dissolved with stirring in THF (5 mL) followed by the addition of aq 10% sodium bicarbonate (2.098 g, 2.497 mmol) and then CBz-C1 (285 μL, 0.999 mmol). The reaction mixture was stirred for 2 h, after which time it was diluted with EtOAc (20 mL), and washed with brine (5 mL), and the organic layer was dried over Na₂SO₄, filtered, and concentrated. The residue was purified over a Biotage SNAP Ultra silica gel column (10 g) eluting with a 10 CV gradient of 0 to 100% EtOAc in heptane to provide the CBz-protected intermediate (276 mg, 0.366 mmol, 73%) after combining the desired fractions, concentration, and drying under vacuum.

The CBz protected intermediate was dissolved with stirring in 1,4-dioxane (5 mL) and water (2 mL) at room temperature followed by the addition of 2,6-lutidine (145 μL, 1.249 mmol), osmium tetroxide (235 μL, 0.03 mmol) and sodium periodate (427 mg, 1.998 mmol). The reaction mixture was stirred for 6 h, after which time it was diluted with EtOAc (10 mL), washed with water (5 mL), 1N HCl (5 mL), and then brine (5 mL). The organic layer was filtered over a pad of silica gel (5 g), eluting with EtOAc (20 mL), and the filtrate was concentrated and dried under vacuum to provide 284 (272 mg, 0.359 mmol, 72.0%) (MW_{Calc}+Na=779.27; MW_{Obs}=779.25).

To a stirred solution of (1R,2R)-3-(4-acetoxy-3,5-dimethylbenzamido)-1-((2R,3R,4S,6S)-4-acetoxy-3-(((benzyloxy)carbonyl)amino)-6-(methoxycarbonyl)-6-(2-oxoethyl)tetrahydro-2H-pyran-2-yl)propane-1,2-diyl diacetate (284, 250 mg, 0.33 mmol) in DCE (5 mL) at room temperature was added tert-butyl (S)-9-oxa-2,6-diazaspiro[4.5]decane-2-carboxylate (61, 140 mg, 0.578 mmol), acetic acid (142 μl, 2.478 mmol) and then dried 4A molecular sieves (500 mg). The suspension was stirred for 3 h, after which time sodium triacetoxyborohydride (140 mg, 0.661 mmol) was added. The reaction mixture was stirred for 2 h, after which time the mixture was quenched with sat. NaHCO₃ (5 mL) and diluted with EtOAc (10 mL). The mixture was filtered over a pad of Celite (5 g) eluting with EtOAc (2×5 mL ea), and the combined filtrate was washed with brine (5 mL). The organic layer was dried over Na₂SO₄, filtered, and concentrated. The residue was purified over a Biotage SNAP Ultra silica gel column (10 g) eluting with a 10 CV gradient of 0 to 10% MeOH in DCM, then 3 CV 10% MeOH in DCM to provide the desired intermediate (230 mg, 0.235 mmol, 71%) (MW_{Calc}+Na=983.44; MW_{Obs}=983.43) after combining the desired fractions, concentration, and drying under vacuum.

The intermediate from above was dissolved with stirring in EtOH (2 mL) and EtOAc (2 mL) at room temperature followed by the addition of 10% Pd/C (200 mg), and then purged with a H2 atmosphere. The reaction mixture was stirred under a H₂ atmosphere (balloon pressure) for 16 h, after which time the mixture was purged several times with N₂ gas, filtered over a pad of Celite (5 g), eluting with EtOAc (5×3 mL ea). The filtrate was concentrated to provide the free amine (167 mg, 0.197 mmol, 60%) (MW_{Calc}+H=849.41; MW_{Obs}=849.45) after drying under vacuum.

To a stirred solution of (1R,2R)-3-(4-acetoxy-3,5-dimethylbenzamido)-1-((2R,3R,4S,6R)-4-acetoxy-3-amino-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)propane-1,2-diyl diacetate (60 mg, 0.071 mmol) and 2-azidoacetic acid (14.3 mg, 0.141 mmol) in MeCN (1 mL) at room temperature were added Hunig's base (37.0 μL, 0.212 mmol), and HATU (53.7 mg, 0.141 mmol). The reaction mixture was stirred for 30 min, after which time the mixture was quenched with water (2 mL) and diluted with EtOAc (5 mL). The layers were separated, and the organic layer was washed with sat NaHCO₃ (2 mL), brine (2 mL), and the organic layer was dried over Na₂SO₄, filtered, and concentrated. The residue was purified over a Biotage SNAP Ultra silica gel column (10 g) eluting with a 10 CV gradient of 0 to 100% EtOAc in heptane to provide compound 285 (65 mg, 0.070 mmol, 99% yield) (MW_{Calc}+4H/4=1004.24; MW_{Obs}=1004.78) after combining the desired fractions, concentration, and drying under vacuum.

To a stirred solution of (1R,2R)-3-(4-acetoxy-3,5-dimethylbenzamido)-1-((2R,3R,4S,6R)-4-acetoxy-3-(2-azidoacetamido)-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)propane-1,2-diyl diacetate (285, 100 mg, 0.108 mmol) and N1,N3,N5-tris(1-((1R,4R)-4-ethynylcyclohexyl)-1-oxo-6,9,12-trioxa-2-azapentadecan-15-yl)benzene-1,3,5-tricarboxamide (283, 70 mg, 0.029 mmol) in a 1:1:1 mixture of t-BuOH:water:THF (3 mL) at room temperature was added L-ascorbic acid sodium salt (51.2 mg, 0.258 mmol) and solid Fehling A solution (Copper sulfate pentahydrate, 20.7 μL, 0.086 mmol). The reaction mixture was stirred for 4 h, after which time the mixture as diluted with a 1:1 solution of THF:EtOAC (5 mL). The mixture was filtered over a pad of Celite (2 g), eluted with a 1:1 solution of THF:EtOAC (3 mL). The filtrate was concentrated. The residue was dissolved in a 1:1 solution of DCM/MeOH (3 mL), filtered of a pad of Celite (2 g), eluted with a 1:1 solution of DCM/MeOH (3 mL). The filtrate was concentrated to dry. The residue was dissolved in MeOH (2 mL) at room temperature was added 1 N NaOH (1.0, 1.00 mmol). The reaction mixture was stirred for 3 h, after which time the mixture was concentrated, and then neutralized with 1 N HCl (1.0 mL, 1.0 mmol). The resultant mixture was concentrated, dissolved in MeOH (1 mL), filtered, and purified by HPLC to provide A-369 (11 mg, 0.003 mmol, 11% yield) (MW_{Calc}+4H/4=871.45; MW_{Obs}=871.72) after combining the desired fractions, concentration, and drying under vacuum.

### Preparation of A-370

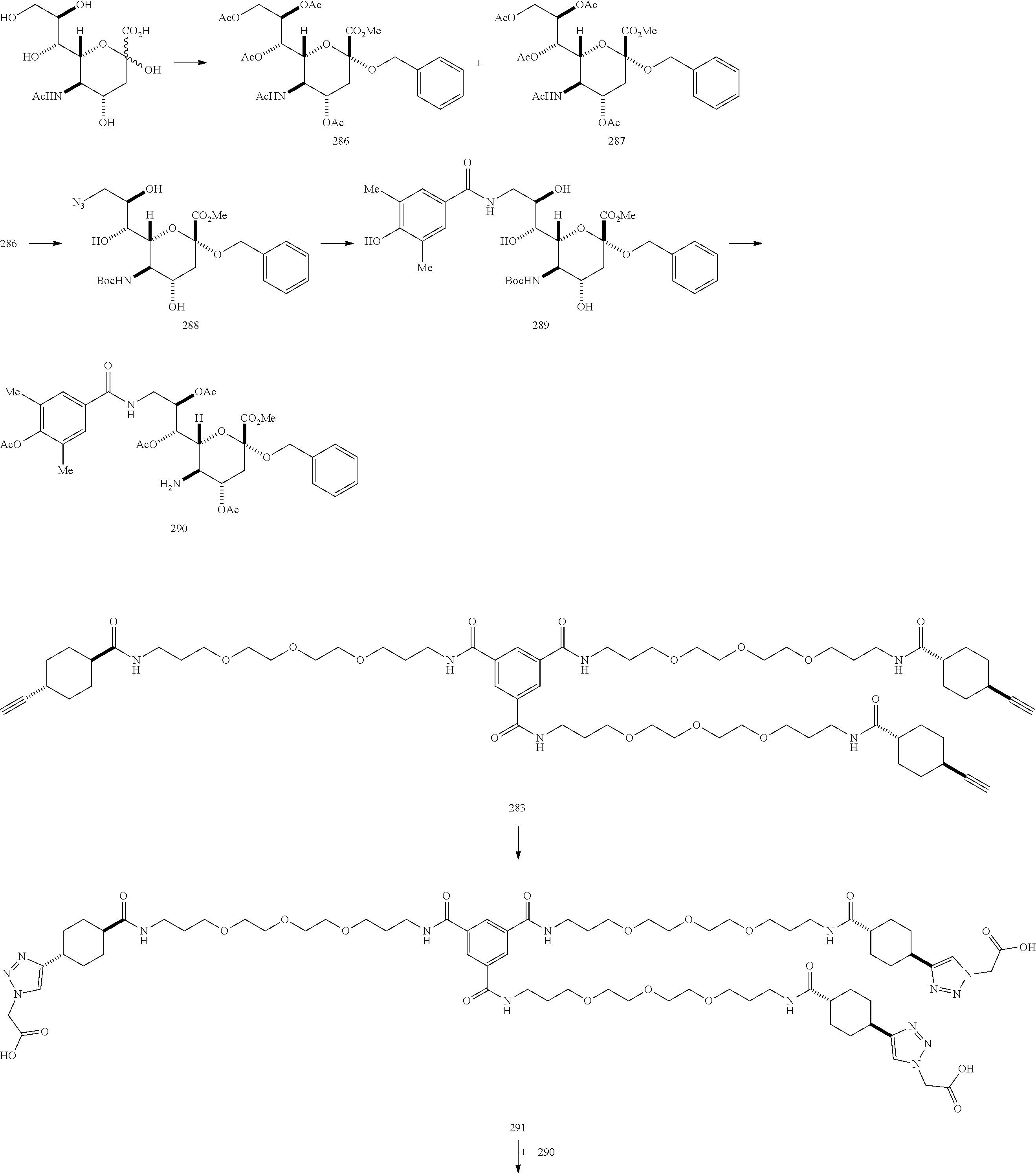

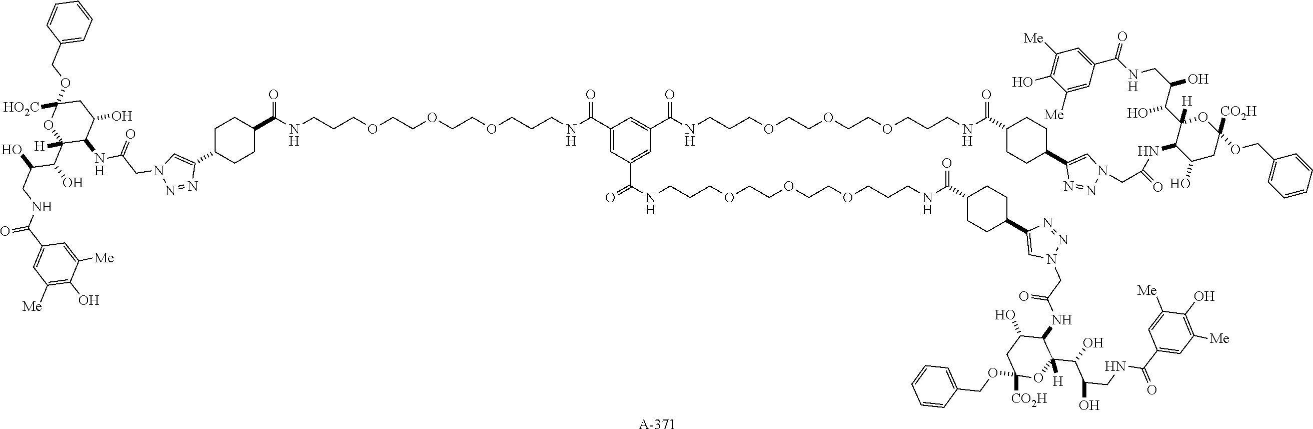

To a stirred solution of (4S,5R,6R)-5-acetamido-2,4-dihydroxy-6-((1R,2R)-1,2,3-trihydroxypropyl)-tetrahydro-2H-pyran-2-carboxylic acid (20 g, 64.668 mmol) in methanol (410 mL) at room temperature was added Dowex 50W×4 hydrogen form (20 g). The reaction mixture was stirred for 40 min, after which time the mixture was filtered, the filter pad was rinsed with MeOH (2×20 mL ea), and the filtrate was concentrated followed by azeotroping to dry with MeCN (2×40 mL ea). The residue was dissolved with stirring in pyridine (78 mL) and DCM (167 mL) at 0° C., followed by the addition of DMAP (1.58 g, 12.93 mmol), and a dropwise addition of Ac₂O (48.8 mL, 517.35 mmol) over 15 min. The reaction mixture was warmed to room temperature and stirred for 16 h. The completed reaction was quenched with water (200 mL), diluted with EtOAc (200 mL), and the layers were separated. The aqueous layer was extracted once with EtOAc (100 mL), and the combined organic layers were washed with 3N HCl (300 mL), 1N HCl (50 mL), carefully with sat. NaHCO₃ (3×100 mL ea), and a 1:1 solution of water: brine. The resultant organic layer was dried over Na₂SO₄, filtered, concentrated, and dried under vacuum to provide the crude desired product (32.33 g, 60.60 mmol, 94%) (MW_{Calc}+Na=556.17; MW_{Obs}=556.30), which was used directly in the next reaction.

To a stirred solution of crude (1S,2R)-1-((2R,3R,4S)-3-acetamido-4,6-diacetoxy-6-(methoxycarbonyl)-tetrahydro-2H-pyran-2-yl)propane-1,2,3-triyl triacetate (8 g, 14.996 mmol) in acetyl chloride (112 mL, 1574.6 mmol) at 0° C. was slowly added methanol (16.02 ml, 395.9 mmol) over 15 min maintaining the temperature below 6° C. The reaction mixture was slowly warmed to room temperature stirring for a total of 16 h., The completed reaction was concentrated, azeotroped to dry with EtOAc (3×50 mL ea), and the residue was dried under vacuum to provide the desired crude product (7.65 g, 15.0 mmol, 100%).

To a stirred solution of crude (1S,2R)-1-((2R,3R,4S,6R)-3-acetamido-4-acetoxy-6-chloro-6-(methoxycarbonyl)tetrahydro-2H-pyran-2-yl)propane-1,2,3-triyl triacetate (14.18 g, 27.81 mmol) in benzyl alcohol (33 mL, 317.373 mmol) at room temperature was added activated 4 Å molecular sieves (7 g, 320 mesh). The mixture was stirred for 16 h, after which time silver salicylate (7.20 g, 29.39 mmol) was added portion wise over 5 min maintaining at room temperature. The reaction mixture was stirred for 4 h, after which time the mixture was s diluted with EtOAc (50 mL), filtered through a pad of Celite (23 g), and eluted with EtOAc (500 mL). The filtrate was washed with 15 wt % Na₂S₂O₃ (100 mL), NaHCO₃ (100 mL), and then brine (50 mL). The organic layer was dried over Na₂SO₄, filtered, concentrated and then purified over a Biotage SNAP KP silica gel column (340 g) eluting with 1 CV heptane, 1 CV gradient of 0 to 5% EtOAc in heptane, 1 CV 5% EtOAc in heptane, a 5 CV gradient of 5 to 50% EtOAc in heptane, a 5 CV gradient of 50 to 100% EtOAc in heptane, and a 5 CV gradient of 0 to 30% MeOH in EtOAc to provide compound 286 (11.4 g, 19.60 mmol, 71%) (MW_{Calc}+Na=604.21; MW_{Obs}=604.28) and 287 (2.8 g, 4.81 mmol, 17%) (MW_{Calc}+Na=604.21; MW_{Obs}=604.28) after combining the desired fractions, concentration, and drying under vacuum.

To a stirred solution of (1S,2R)-1-((2R,3R,4S,6R)-3-acetamido-4-acetoxy-6-(benzyloxy)-6-(methoxycarbonyl)tetrahydro-2H-pyran-2-yl)propane-1,2,3-triyl triacetate (286, 28.8 g, 49.52 mmol) in anhydrous THF (100 mL) at room temperature were added triethylamine (10.35 mL, 74.282 mmol), DMAP (1.210 g, 9.90 mmol), and BOC-anhydride (17.25 mL, 74.28 mmol). The reaction mixture was stirred 96 h, after which time added Boc-anhydride (15 g, 68.73 mmol), and continued to stir for an additional 24 h. The completed reaction was diluted with EtOAc (200 mL) and washed with 1 N HCl (100 mL) and then brine (100 mL). The combined aqueous layers were extracted with EtOAc (50 mL), and the combined organic layers were dried over Na₂SO₄, filtered, and concentrated. The resultant, dark red-brown oil diluted with DCM (50 mL), mixed with silica gel (100 g, containing 50 mL DCM), and stirred for 16 h. The slurry was poured over a column of silica gel (250 g, previously slurried in DCM), and eluted with 20% EtOAc in DCM (1 L), 40% EtOAc in DCM (1.5 L), 60% EtOAc in DCM (1.5 L), and 100% EtOAc (1.5 L), collecting 500 mL fractions to provide the crude Boc-protected intermediate after combining the desired fractions, concentration, and drying under vacuum. (MW_{Calc}+Na=704.21; MW_{Obs}=704.22).

To a stirred solution of (1S,2R)-1-((2R,3R,4S,6R)-4-acetoxy-6-(benzyloxy)-3-(N-(tert-butoxycarbonyl)-acetamido)-6-(methoxycarbonyl)tetrahydro-2H-pyran-2-yl)propane-1,2,3-triyl triacetate (33.8 g, 49.583 mmol) in anhydrous methanol (100 mL) at 0° C. was added a 25 wt % methanol solution of sodium methoxide (100 mL, 437.308 mmol). The reaction mixture was warmed to room temperature and stirred for 16 h. The completed reaction was quenched with acetic acid (25.02 mL, 437 mmol), stirred for 10 minutes, and then concentrated under reduced pressure. The resultant solid suspended in EtOAc (100 mL) stirred for 30 min, and then filtered, rinsing the filter cake with EtOAc (4×20 mL ea). The combined filtrates were then concentrated and dried under vacuum to provide the desired deacylated product as a reddish-orange foamy solid. (MW_{Calc}+Na=494.21; MW_{Obs}=494.21).

To a stirred solution of (2R,4S,5R,6R)-methyl 2-(benzyloxy)-5-((tert-butoxycarbonyl)amino)-4-hydroxy-6-((1R,2R)-1,2,3-trihydroxypropyl)tetrahydro-2H-pyran-2-carboxylate (26.3 g, 55.78 mmol) in anhydrous DMF (300 mL) at room temperature were added sodium azide (18.13 g, 278.9 mmol) and carbon tetrabromide (27.7 g, 83.67 mmol). The mixture was cooled to <10° C. followed by a piecemeal addition of triphenylphosphine (21.95 g, 83.669 mmol) over 5 min. The reaction mixture was allowed slowly warm to room temperature, and stirred for 72 h. The completed reaction was quenched with water (200 mL), and extracted with EtOAc (2×200 mL ea). The combined organic layers were washed with brine (50 mL), concentrated, and purified over a Biotage SNAP KP silica gel column (340 g) eluting with 1 CV DCM, a 10 CV gradient of 0 to 100% EtOAc in DCM, and 2 CV EtOAc to provide compound 288 (12.73 g, 25.60 mmol, 46%) after combining the desired fractions, concentration, and drying under vacuum.

To a stirred solution of (2R,4S,5R,6R)-methyl 6-((1R,2R)-3-azido-1,2-dihydroxypropyl)-2-(benzyloxy)-5-((tert-butoxycarbonyl)amino)-4-hydroxytetrahydro-2H-pyran-2-carboxylate (288, 3.024 g, 6.09 mmol) in THF (65.9 mL) and water (4.39 mL) at 0° C., was added dropwise 1 M trimethylphosphine in THF (18.27 mL, 18.27 mmol) over 10 min. The reaction was warmed to room temperature, and stirred for 72 h. The completed reaction was concentrated, and azeotroped to dry with acetonitrile (3×20 mL ea) to provide the desired amine, which was used in the next reaction without further purification.

To a stirred solution of 4-hydroxy-3,5-dimethylbenzoic acid (1.216 g, 7.32 mmol) in DCM (15.70 mL) at 0° C. was added DIPEA (3.43 mL, 19.641 mmol), followed by 3H-[1,2,3]triazolo[4,5-b]pyridin-3-ol (1.162 g, 8.54 mmol), and N1-((ethylimino)methylene)-N3,N3-dimethylpropane-1,3-diamine hydrochloride (1.871 g, 9.76 mmol). The mixture was stirred at 0° C. for 15 min, after which time was added dropwise a solution of (2R,4S,5R,6R)-methyl 6-((1R,2R)-3-amino-1,2-dihydroxypropyl)-2-(benzyloxy)-5-((tert-butoxycarbonyl)amino)-4-hydroxytetrahydro-2H-pyran-2-carboxylate (2.87 g, 6.10 mmol) in DCM (55.7 mL) over 15 min. The reaction mixture was warmed to room temperature and stirred for 16 h. The completed reaction mixture was diluted with a 1:1 ratio of EtOAc to Et20 (40 ml) and washed with sat NaHCO₃ (20 mL). The combined aqueous layers were extracted with a 1:1 ratio of EtOAc to Et2O (30 mL), and the combined organic layers were dried over Na₂SO₄, filtered, and concentrated. The residue was purified over a Biotage SNAP KP silica gel column (50 g) eluting with a 10 CV gradient of 0 to 3% MeOH to provide compound 289 (3.12 g, 5.04 mmol, 83%) (MW_{Calc}+H=619.28; MW_{Obs}=619.50) after combining the desired fractions, concentration, and drying under vacuum.

To a stirred solution of (2R,4S,5R,6R)-methyl 2-(benzyloxy)-5-((tert-butoxycarbonyl)amino)-6-((1R,2R)-1,2-dihydroxy-3-(4-hydroxy-3,5-dimethylbenzamido)propyl)-4-hydroxytetrahydro-2H-pyran-2-carboxylate (289, 2.33 g, 3.766 mmol) in pyridine (10.42 mL, 128.80 mmol) at room temperature was added acetic anhydride (10.41 mL, 110.35 mmol). Reaction mixture was stirred for 18 h, after which time the mixture was diluted with EtOAc (100 mL) followed by the addition of 1 N HCl until the solution reached pH 5. The resulting mixture was extracted with EtOAc (3×20 mL), and the combined organic layers were washed with sat. NH₄Cl (20 mL) and brine (20 mL). The organic layer was dried with Na₂SO₄, filtered, concentrated, and dried under vacuum to provide the desired fully protected desire product (3.37 g, 3.00 mmol, 70%) (MW_{Calc}+H=787.32; MW_{Obs}=787.64), which was used in the next step without further purification.

To a stirred solution of (1R,2R)-3-(4-acetoxy-3,5-dimethylbenzamido)-1-((2R,3R,4S,6R)-4-acetoxy-6-(benzyloxy)-3-((tert-butoxycarbonyl)amino)-6-(methoxycarbonyl)tetrahydro-2H-pyran-2-yl)propane-1,2-diyl diacetate (3.37 g, 4.283 mmol) in DCM (11.82 mL) at room temperature was added TFA (11.85 mL, 153.76 mmol). The reaction mixture was stirred for 15 min, after which time the mixture was diluted with toluene (20 mL), concentrated, azeotroped to dry with toluene (3×10 mL ea), and dried under vacuum. The residue was purified over a Biotage SNAP Ultra silica gel column (50 g) eluting with a 10 CV gradient of 50 to 100% EtOAc in heptane, 2 CV EtOAC, and a 10 CV gradient of 0 to 20% MeOH in DCM to provide compound 290 (2.92 g, 4.25 mmol, 99%) (MW_{Calc}+2H=688.27; MW_{Obs}=688.56) after combining the desired fractions, concentration, and drying under vacuum.

To a stirred solution of N1,N3,N5-tris(1-((1R,4R)-4-ethynylcyclohexyl)-1-oxo-6,9,12-trioxa-2-azapentadecan-15-yl)benzene-1,3,5-tricarboxamide (283, 660 mg, 0.541 mmol) and ethyl 2-azidoacetate (1.242 mL, 2.165 mmol) in THF (15 mL) at room temperature were added tert-butanol (15 mL) and water (15 mL) followed by copper(ii) sulfate pentahydrate (405 mg, 1.624 mmol) and sodium ascorbate (965 mg, 4.871 mmol). The reaction mixture was stirred for 15 min, after which time added sodium ascorbate (965 mg, 4.871 mmol), and stirred for 1 h. The completed reaction was then filtered through a pad of Celite (5 g) eluting with a mixture of 10% MeOH in DCM (2×5 mL ea). The filtrate was concentrated to dry, after which time the residue was sonicated in 10% MeOH in DCM (20 mL), filtered through a pad of Celite (5 g) eluting with DCM (2×10 mL ea). The previous process was repeated. The residue from the second process was purified over a Biotage SNAP Ultra silica gel column (50 g) eluting with a 10 CV gradient of 5 to 15% MeOH in DCM to provide the desired triester (335 mg, 0.208 mmol, 39%) after combining the desired fractions, concentration, and drying under vacuum.

To a stirred solution of triethyl 2,2′,2″-(4,4′,4″-((1R,1′R,1″R,4R,4′R,4″R)-(17,17′,17″-(benzene-1,3,5-triyl)tris(17-oxo-6,9,12-trioxa-2,16-diazaheptadecan-1-oyl))tris(cyclohexane-4,1-diyl))tris(H-1,2,3-triazole-4,1-diyl))triacetate (335 mg, 0.208 mmol) in MeOH (5 mL) at room temperature was added 1 N NaOH (1.251 mL, 1.251 mmol). The reaction mixture was stirred for 16 h, after which time the mixture was neutralized with 1 N HCl (1.00 mL, 1.00 mmol) to pH 5, and concentrated. The residue dissolved in MeOH (1 mL), filtered through 2-micron filter, and eluted with MeOH (2×1 mL ea). The filtrate was diluted to 5.5 mL MeOH and purified by HPLC to provide compound 291 (225 mg, 0.138 mmol, 66%) (MW_{Calc}+3H/3=536.30; MW_{Obs}=536.73) after combining the desired fractions, concentration, and drying under vacuum.

To a stirred solution of (1R,2R)-3-(4-acetoxy-3,5-dimethylbenzamido)-1-((2R,3R,4S,6R)-4-acetoxy-3-amino-6-(benzyloxy)-6-(methoxycarbonyl)tetrahydro-2H-pyran-2-yl)propane-1,2-diyl diacetate (290, 194 mg, 0.283 mmol) and 2,2′,2″-(4,4′,4″-((1R,1′,1″R, 4R,4′W, 4″R)-(17,17′,17″-(benzene-1,3,5-triyl)tris(17-oxo-6,9,12-trioxa-2,16-diazaheptadecan-1-oyl))tris(cyclohexane-4,1-diyl))tris(H-1,2,3-triazole-4,1-diyl))triacetic acid trihydrochloride (291, 140 mg, 0.086 mmol) in DMF (5 mL) at room temperature were added HATU (108 mg, 0.283 mmol) and triethylamine (0.120 mL, 0.858 mmol). The reaction mixture was stirred for 30 min, after which time the completed reaction mixture was concentrated, dried under vacuum, and used in the next step without further purification.

To a stirred solution of the crude protected trivalent compound from the previous reaction (303 mg, 0.086 mmol) in MeOH (5 mL) at room temperature was added 1 N NaOH (2.58 mL, 2.58 mmol). The reaction mixture was stirred for 3 h, after which time the mixture was concentrated, and then neutralized with 1 N HCl (2.147 mL, 2.147 mmol, pH 4-5). The resultant mixture was concentrated, dissolved in MeOH (1 mL), filtered, and purified by HPLC (0.1% HCO₂H) to provide A-370 (181 mg, 0.055 mmol, 64%) (MW_{Calc}+3H/3=994.47; MW_{Obs}=994.93) as tri-formic acid salt after combining the desired fractions, concentration, and drying under vacuum.

### Preparation of A-371

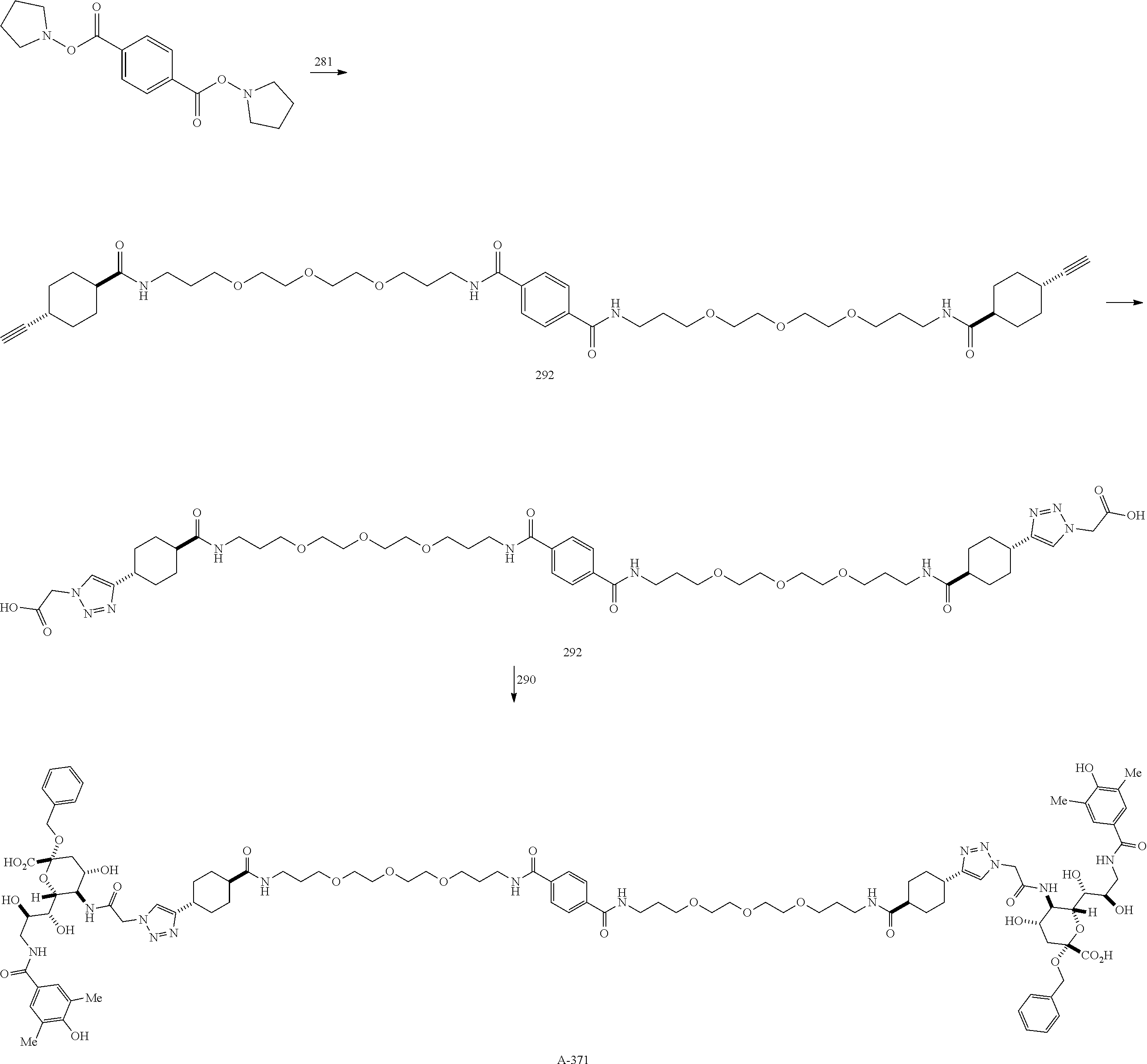

To a stirred solution of terephthaloyl dichloride (5.0 g, 24.628 mmol) in DCM (100 mL) at 0° C. was added 1-hydroxypyrrolidine-2,5-dione (7.09 g, 61.57 mmol) followed by dropwise addition of TEA (10.30 ml, 73.884 mmol). The mixture was stirred for 1 h, warmed to room temperature, and then stirred for 48 h. The completed reaction mixture was filtered; the filter cake washed with DCM (70 mL), and the solid was suspended in DCM (100 mL). After stirring for 10 min, the suspension was filtered, and the filter cake was dried under vacuum to give the desired product (6.45 g 17.90 mmol, 73%).

To a stirred solution of bis(2,5-dioxopyrrolidin-1-yl) terephthalate (42 mg, 0.117 mmol) and (1R,4R)—N-(3-(2-(2-(3-aminopropoxy)ethoxy)ethoxy)propyl)-4-ethynylcyclohexanecarboxamide (281, 100 mg, 0.282 mmol) in DMF (5 mL) at room temperature was added TEA (1 ml, 7.18 mmol) and DMAP (142 mg, 1.166 mmol). The reaction mixture was stirred for 16 h, after which time the completed reaction mixture was concentrated to dry, and purified directly over a Biotage SNAP Ultra silica gel column (10 g) eluting with a 5 CV gradient of 5 to 10% MeOH in DCM, and 4 CV 10% MeOH in DCM to provide compound 292 (48.0 mg, 0.057 mmol, 49%) (MW_{Calc}+H=839.51; MW_{Obs}=839.67) after combining the desired fractions, concentration, and drying under vacuum.

To a stirred solution of N1,N4-bis(1-((1R,4R)-4-ethynylcyclohexyl)-1-oxo-6,9,12-trioxa-2-azapentadecan-15-yl)terephthalamide (292, 316 mg, 0.377 mmol) and ethyl 2-azidoacetate (0.648 mL, 1.13 mmol) in THF (5 mL) at room temperature were added tert-butanol (5 mL, 52.279 mmol) and water (5 mL, 277.542 mmol) at rt was added copper(ii) sulfate pentahydrate (188 mg, 0.753 mmol) and sodium ascorbate (448 mg, 2.26 mmol). The reaction mixture as stirred for 15 min, after which time sodium ascorbate (448 mg, 2.26 mmol), and the mixture was stirred for 1 h. The completed reaction was filtered through a pad of Celite (5 g), eluting the pad with 10% MeOH in EtOAc (3×10 mL ea). The combined filtrates were concentrated to dry, and the residue was purified directly by HPLC to the desired diester (205 mg, 0.187 mmol, 50%) (MW_{Calc}+H=1097.62; MW_{Obs}=1097.75) after combining the desired fractions, concentration, and drying under vacuum.

To a stirred solution of diethyl 2,2′-(4,4′-((1R,1′R,4R,4′R)-(17,17′-(1,4-phenylene)bis(17-oxo-6,9,12-trioxa-2,16-diazaheptadecan-1-oyl))bis(cyclohexane-4,1-diyl))bis(1H-1,2,3-triazole-4,1-diyl))diacetate (205 mg, 0.187 mmol) in MeOH (4 mL) at room temperature was added 1 N NaOH (1.00 mL, 1.00 mmol). The reaction mixture was stirred for 16 h, after which time it was neutralized with 1 N HCl (0.934 mL, 0.934 mmol). The mixture was concentrated to dryness, then dissolved in MeOH (2 mL), filtered through a 2-micron filter rinsing with MeOH (3×1 mL ea). The filtrate was purified by HPLC to provide compound 293 (189 mg, 0.170 mmol, 91%) (MW_{Calc}+H=1041.55; MW_{Obs}=1041.66) as the HCl salt after combining the desired fractions, concentration, and drying under vacuum until MeOH free.

To a stirred solution of (1R,2R)-3-(4-acetoxy-3,5-dimethylbenzamido)-1-((2R,3R,4S,6R)-4-acetoxy-3-amino-6-(benzyloxy)-6-(methoxycarbonyl)tetrahydro-2H-pyran-2-yl)propane-1,2-diyl diacetate (290, 121 mg, 0.176 mmol) and 2,2′-(4,4′-(((1R,1′R,4R,4′R)-(17,17′-(1,4-phenylene)bis(17-oxo-6,9,12-trioxa-2,16-diazaheptadecan-1-oyl))bis(cyclohexane-4,1-diyl))bis(1H-1,2,3-triazole-4,1-diyl))diacetic acid dihydrochloride (293, 89 mg, 0.08 mmol) in DMF (3.56 mL) at room temperature was added HATU (66.8 mg, 0.176 mmol) and triethylamine (0.111 mL, 0.799 mmol). The reaction mixture was stirred for 30 min, after which time it was concentrated to dry, and the residue was dissolved in MeOH (2 mL) and purified by HPLC to provide the desired protected dimer (69 mg, 0.029 mmol, 36%) (MW_{Calc}/2+H=1190.04; MW_{Obs}=1190.36) after combining the desired fractions, concentration, and drying under vacuum until MeOH free.

To a stirred solution of the above dimer (145 mg, 0.061 mmol) in MeOH (4 mL) at room temperature was added 1 N NaOH (1.3 ml, 1.30 mmol). The reaction mixture was stirred for 16 h, after which time it was neutralized with 1 N HCl (1.1 ml, 1.10 mmol, pH=4), filtered and purified directly by HPLC to provide A-371 (106 mg, 0.050 mmol, 83%) (MW_{Calc}/2+H=1007.48; MW_{Obs}=1007.88) as the diformate salt after combining the desired fractions, concentration, and drying under vacuum until MeOH free.

### Lipid Conjugate Analogs

### Preparation of A-372

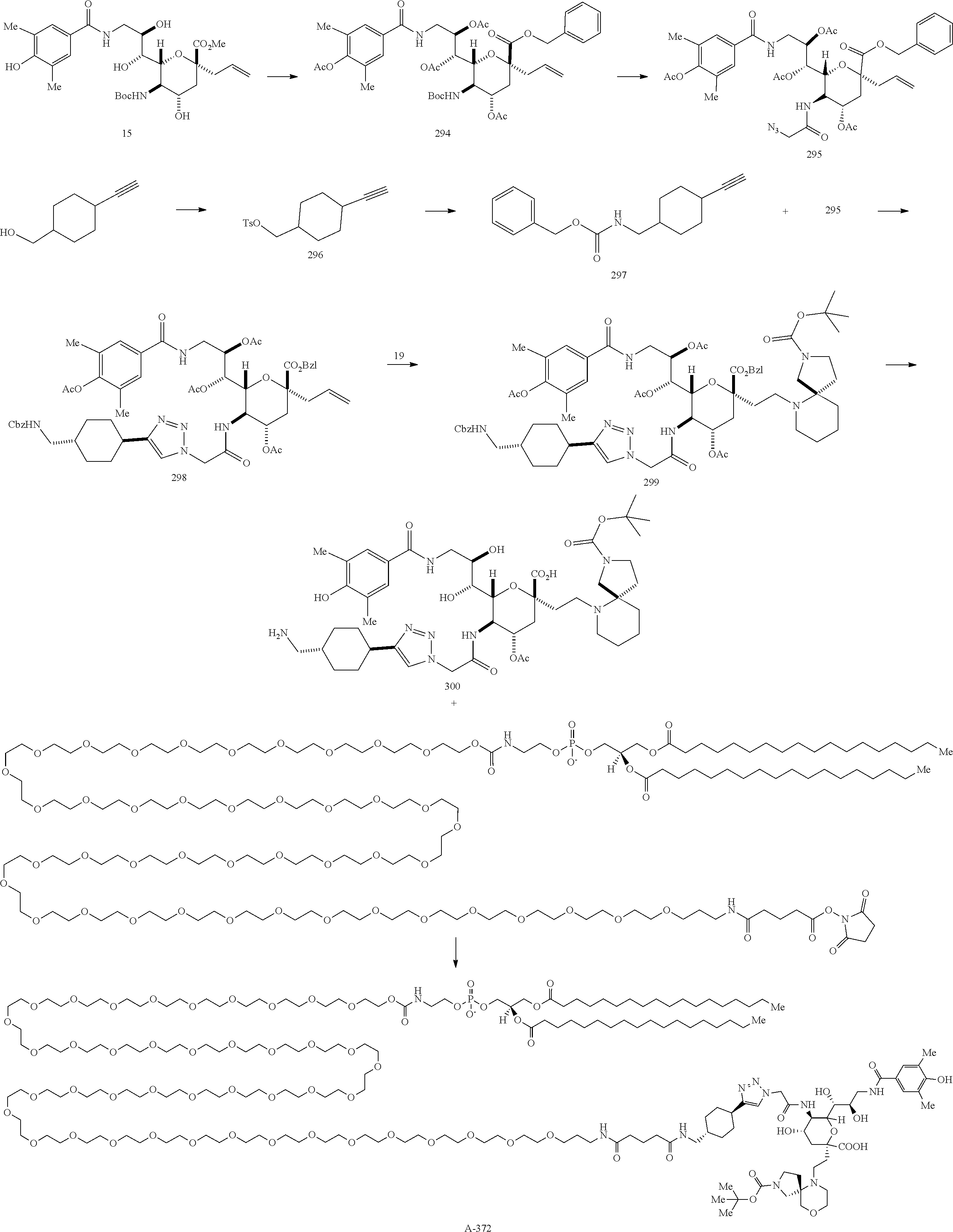

To a stirred solution of methyl (2R,4S,5R,6R)-2-allyl-5-((tert-butoxycarbonyl)amino)-6-((1R,2R)-1,2-dihydroxy-3-(4-hydroxy-3,5-dimethylbenzamido) propyl)-4-hydroxytetrahydro-2H-pyran-2-carboxylate (15, 1.4 g, 2.533 mmol) in MeOH (6 mL) at room temperature was added 1 N NaOH (5.07 mL, 5.07 mmol). The reaction mixture was stirred over 24 h, after which time it was acidified with 1N HCl (6 mL, 6.0 mmol), and extracted extensively with EtOAc (3×10 mL ea). The combined organic layers were washed with brine (1 mL), dried over Na₂SO₄, filtered, and concentrated. The residue was dissolved with stirring in pyridine (4 mL, 49.46 mmol) at room temperature followed by the addition of acetic anhydride (4 mL, 42.23 mmol). The reaction mixture was stirred for 24 h, after which time the mixture was neutralized with 1 N HCl (˜40 mL) and extracted with EtOAc (3×30 mL ea). The combined organic layers were washed with 1N HCl (20 mL) and brine (10 mL), and then dried over Na₂SO₄, filtered, and concentrated to dry. The resultant oil was dissolved with stirring in DMF (6 mL) at 0° C. followed by the addition of potassium carbonate (0.350 g, 2.533 mmol), and then benzyl bromide (0.452 mL, 3.80 mmol). The final reaction mixture was stirred at 0° C. for 3 h, after which time it was diluted with EtOAc (20 mL) and water (10 mL). The layers were separated, and the aqueous layer was extracted with EtOAc (2×10 mL ea). The combined organic layers were washed with brine (10 mL), dried over Na₂SO₄, filtered, and concentrated to dry. The residue was purified over a Biotage SNAP Ultra silica gel column (10 g) eluting with a 5 CV gradient of 0 to 75% EtOAc in heptane to provide compound 294 (890 mg, 1.117 mmol, 44%) (MW_{Calc}+Na=819.34; MW_{Obs}=819.26) after combining the desired fractions, concentration, and drying under vacuum.

To a stirred solution of (1R,2R)-3-(4-acetoxy-3,5-dimethylbenzamido)-1-((2R,3R,4S,6R)-4-acetoxy-6-allyl-6-((benzyloxy)carbonyl)-3-((tert-butoxycarbonyl)amino)tetrahydro-2H-pyran-2-yl)propane-1,2-diyl diacetate (294, 800 mg, 1.004 mmol) in DCM (10 mL) at room temperature was added TFA (1 mL, 13.05 mmol). The reaction mixture was stirred for 30 min, after which time the mixture was concentrated, and azeotroped to dry with toluene (4×5 mL ea). The residue was dissolved with stirring in MeCN (5 mL) at room temperature followed by the addition of 2-azidoacetic acid (178 mg, 1.757 mmol), triethylamine (364 μL, 2.61 mmol), and then HATU (573 mg, 1.506 mmol). The reaction mixture was stirred for 5 h, after which time it was diluted with water (10 mL), and then the mixture was extracted with EtOAc (3×10 mL). The combined organic layers were washed with brine (10 mL), and dried over Na₂SO₄, filtered, and concentrated to dry. The residue was purified over a Biotage SNAP Ultra silica gel column (10 g) eluting with a 10 CV gradient of 0 to 100% EtOAc in heptane to provide compound 295 (653 mg, 0.837 mmol, 83%) (MW_{Calc}+H=780.30; MW_{Obs}=780.26) after combining the desired fractions, concentration, and drying under vacuum.

To a stirred solution of (4-ethynylcyclohexyl)methanol (1.0 g, 7.236 mmol) in DCM (15.00 mL) at room temperature were added pyridine (1.17 mL, 14.471 mmol), DMAP (0.088 g, 0.724 mmol) 233.126 mmol) and p-toluenesulfonyl chloride (1.517 g, 7.959 mmol). The mixture was stirred for 16 h, after which time it was acidified with 1 M HCl (pH<3), the layers separated, and the aqueous layer was extracted with DCM (2×10 mL) and EtOAc (10 mL). The combined organic layers were dried over Na₂SO₄, filtered and concentrated to give a colorless oil. The oil was purified over a Biotage SNAP Ultra silica gel column (50 g) eluting 10 CV of 10% EtOAc in heptane to provide the desired tosylate 296 (2.0 g, 6.84 mmol, 95%) after combining the desired fractions, concentration, and drying under vacuum.

To a stirred solution of (4-ethynylcyclohexyl)methyl 4-methylbenzenesulfonate (296, 2.0 g, 6.84 mmol) in DMF (0.53 mL) at room temperature was added sodium azide (1.334 g, 20.52 mmol) followed by warming to 60° C. The reaction mixture was stirred at 60° C. for 16 h, after which time it was cooled to room temperature, and then poured into a 1:1 solution of sat. NaHCO₃ in water (10 mL). The mixture was extracted with MTBE (10 mL) and EtOAc (10 mL), and the combined organic layers were washed with water (2×5 mL). The organic layer was concentrated to dry, and the resulting residue was dissolved with stirring in THF (10 mL) and water (1 mL) followed by the addition of 1 M trimethylphosphine in THF (10.26 mL, 10.26 mmol). The reaction mixture was stirred for 3 h, after which time it was concentrated to dry under high vacuum, and then azeotroped to dry with toluene (2×5 mL ea). The crude amine was dissolved with stirring in THF (10 mL) at room temperature followed by the addition of triethylamine (1.907 mL, 13.68 mmol), and then benzyl chloroformate (2.280 ml, 6.84 mmol). The final reaction mixture was stirred for 3 h, after which time the mixture was concentrated, and purified directly over a Biotage SNAP Ultra silica gel column (25 g) eluting with a 10 CV gradient of 0 to 25% EtOAc in heptane to provide compound 297 (1.6 g, 5.90 mmol, 86%) after combining the desired fractions, concentration, and drying under vacuum.

To a stirred solution of (1R,2R)-3-(4-acetoxy-3,5-dimethylbenzamido)-1-((2R,3R,4S,6R)-4-acetoxy-6-allyl-3-(2-azidoacetamido)-6-((benzyloxy)carbonyl)tetrahydro-2H-pyran-2-yl)propane-1,2-diyl diacetate (295, 616 mg, 0.790 mmol) and benzyl ((4-ethynylcyclohexyl)methyl)carbamate (297, 300 mg, 1.105 mmol) in a 1:1 solution of THF in MeCN (2.4 mL) and water (2 mL) at room temperature were added L-ascorbic acid sodium salt (280 mg, 1.413 mmol) and copper(II) sulfate (240 mg, 1.503 mmol), and then copper(I) iodide (20 mg, 0.105 mmol). The reaction mixture was stirred for 1 h, after which time it was diluted with EtOAc (10 mL) and filtered over a plug of silica (5 g) eluting with EtOAc (3×5 mL ea). The filtrate was washed with brine (5 mL), dried over Na₂SO₄, filtered and concentrated. The resultant residue was purified over Biotage SNAP Ultra silica gel column (25 g) eluting with a 10 CV gradient of 0 to 100% EtOAc in heptane, and then a 5 CV gradient of 0 to 7% MeOH in EtOAc to provide compound 298 (328 mg, 0.312 mmol, 40%) (MW_{Calc}+H=1051.40; MW_{Obs}=1051.58) after combining the desired fractions, concentration, and drying under vacuum.

To a stirred solution of (1R,2R)-3-(4-acetoxy-3,5-dimethylbenzamido)-1-((2R,3R,4S,6R)-4-acetoxy-6-allyl-6-((benzyloxy)carbonyl)-3-(2-(4-((1R,4R)-4-((((benzyloxy)carbonyl)amino)methyl)cyclohexyl)-1H-1,2,3-triazol-1-yl)acetamido)tetrahydro-2H-pyran-2-yl)propane-1,2-diyl diacetate (298, 328 mg, 0.312 mmol) in 1,4-dioxane (4 mL) and water (1 mL) at room temperature were added 2,6-lutidine (100 μL, 0.859 mmol), sodium periodate (240 mg, 1.122 mmol), and then osmium tetroxide (100 mg, 0.393 mmol). The reaction mixture was stirred for 30 min, after which time osmium tetroxide (30 mg, 0.118 mmol) was added, and the final mixture was stirred for 3 h. The completed reaction was diluted with EtOAc (20 mL), and the organic layer was washed with sat NaHCO₃ (10 mL) and brine (5 mL). The resultant organic layer was dried over Na₂SO₄, filtered and concentrated to dryness. The crude aldehyde was used in the next step without further purification.

To a stirred solution of crude (1R,2R)-3-(4-acetoxy-3,5-dimethylbenzamido)-1-((2R,3R,4S,6S)-4-acetoxy-6-((benzyloxy)carbonyl)-3-(2-(4-((1R,4R)-4-(((benzyloxy)carbonyl)amino)methyl)cyclohexyl)-1H-1,2,3-triazol-1-yl)acetamido)-6-(2-oxoethyl)tetrahydro-2H-pyran-2-yl)propane-1,2-diyl diacetate (312 mg, 0.296 mmol) in 1,2-DCE (0.6 mL) at room temperature were added tert-butyl (S)-9-oxa-2,6-diazaspiro[4.5]decane-2-carboxylate (19, 120 mg, 0.495 mmol), acetic acid (120 μL, 2.098 mmol) and activated 4 Å molecular sieves (200 mg). The suspension was stirred for 3 h, after which time sodium triacetoxyborohydride (150 mg, 0.707 mmol) followed by stirring for 3 h. The completed reaction was quenched with sat NaHCO₃ (10 mL) and extracted with EtOAc (3×10 mL ea). The combined organic layers were washed with brine (2×5 mL ea), dried over Na₂SO₄, filtered and concentrated. The residue was purified over Biotage SNAP Ultra silica gel column (10 g) eluting with a 10 CV gradient of 0 to 100% EtOAc in heptane to provide compound 299 (367 mg, 0.287 mmol, 97%) (MW_{Calc}+H=1279.61; MW_{Obs}=1279.57) after combining the desired fractions, concentration, and drying under vacuum.

To a stirred solution of (1R,2R)-3-(4-acetoxy-3,5-dimethylbenzamido)-1-((2R,3R,4S,6R)-4-acetoxy-6-((benzyloxy)carbonyl)-3-(2-(4-((1R,4R)-4-((((benzyloxy)carbonyl)amino)methyl)cyclohexyl)-1H-1,2,3-triazol-1-yl)acetamido)-6-(2-((S)-2-(tert-butoxycarbonyl)-9-oxa-2,6-diazaspiro[4.5]decan-6-yl)ethyl)tetrahydro-2H-pyran-2-yl)propane-1,2-diyl diacetate (299, 52 mg, 0.041 mmol) in methanol (1.98 mL) at room temperature was added 10% Pd—C(8.65 mg, 0.081 mmol) followed by purging with H₂ (5×), then placing under a H₂ atmosphere (balloon pressure). The reaction mixture was stirred for 6 h, after which time the mixture was purged with a N2 atmosphere (5×), diluted with MeOH (2 mL) followed by the addition of K₂CO₃ (100 mg, 0.724 mmol). The reaction mixture was stirred for 1 h, followed by the addition of 1 N NaOH (1 mL, 1.0 mmol), and continued stirring for 30 min. The completed reaction was filtered over a pad of Celite (5 g), eluted with MeOH (2×5 mL), and the filtrate was concentrated to dryness. The residue was purified by HPLC to provide compound 300 (29 mg, 0.033 mmol, 80%) (MW_{Calc}+H=887.48; MW_{Obs}=887.56) after combining the desired fractions, concentration, and drying under vacuum.

To a stirred solution of commercially available sodium (R)-2,3-bis(stearoyloxy)propyl (146-((2,5-dioxopyrrolidin-1-yl)oxy)-4,142,146-trioxo-5,8,11,14,17,20,23,26,29,32,35,38,41,44,47,50,53,56,59,62,65,68,71,74,77,80,83,86,89,92,95,98,101,104, 107,110,113,116,119,122,125,128,131,134,137-pentatetracontaoxa-3,141-diazahexatetracontahectyl) phosphate (20.43 mg, 6.764 μmol) in DMF (180 μL) under a N2 atmosphere at room temperature was added a solution of (2R,4S,5R,6R)-5-(2-(4-((1R,4R)-4-(aminomethyl)cyclohexyl)-1H-1,2,3-triazol-1-yl)acetamido)-2-(2-((S)-2-(tert-butoxycarbonyl)-9-oxa-2,6-diazaspiro[4.5]decan-6-yl)ethyl)-6-((1R,2R)-1,2-dihydroxy-3-(4-hydroxy-3,5-dimethylbenzamido)-propyl)-4-hydroxytetrahydro-2H-pyran-2-carboxylic acid (300, 6.0 mg, 6.764 μmol, previously dissolved in DMF and concentrated to dryness under high vacuum) in 0.1 M sodium bicarbonate (1.69 mL, 0.169 mmol). The reaction mixture was stirred for total of 2 h, after which time the residue was dissolved into water (0.1 mL) and purified on a Sep-PAK c18 cartridge. The cartridge was previously conditioned by eluting with MeOH, 30% MeOH in water, 10% MeOH in water, water with 0.1% Hunig's base, and then only water (5 mL ea). The aqueous solution was loaded on the cartridge and eluted with a step gradient of 0 to 100% MeOH in water in 10% intervals (4 mL/step) to provide A-372 (22.1 mg, 5.8 μmol, 86%) after combining the desired fractions, concentration, and drying under vacuum.

### Preparation of A-373

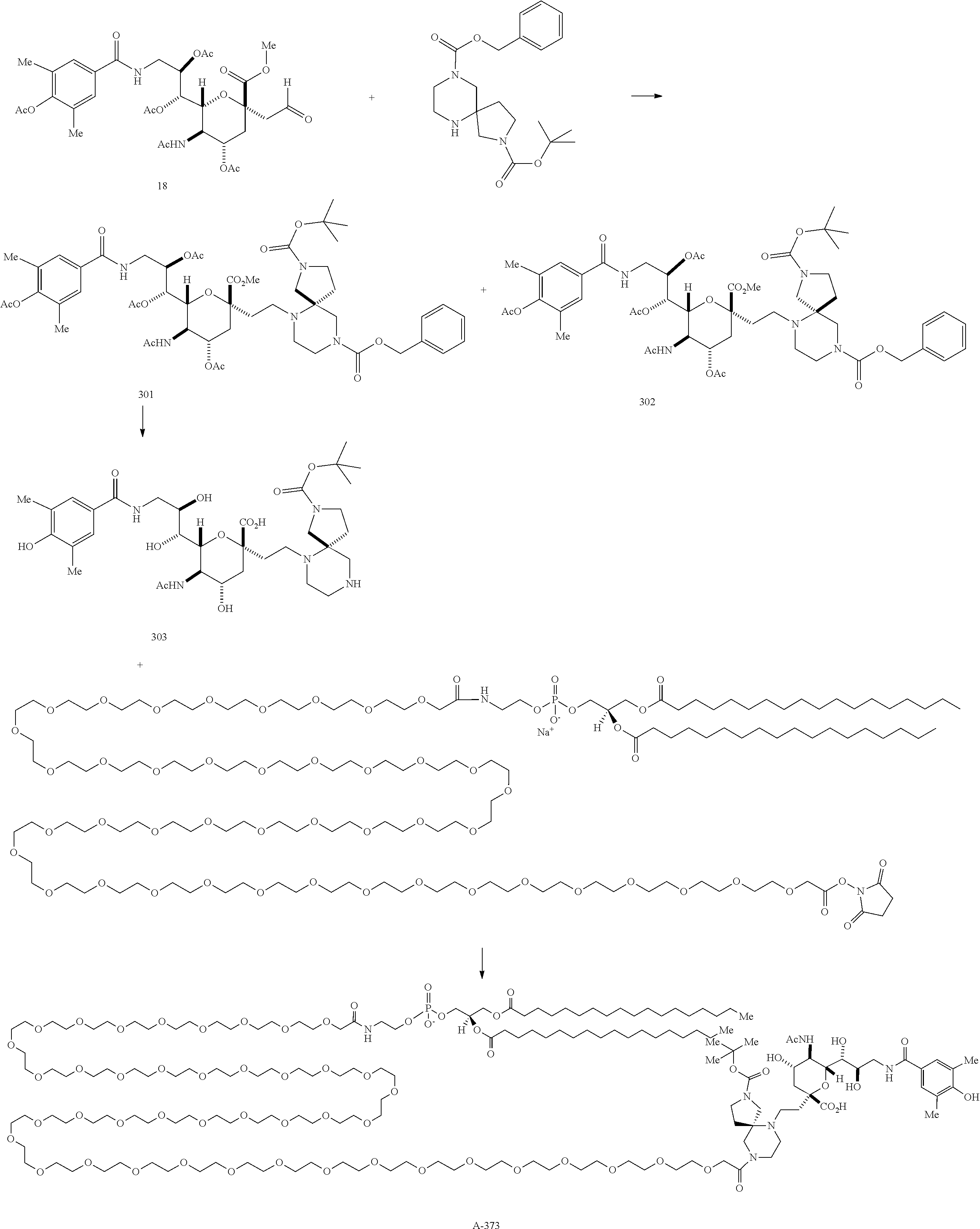

To a stirred solution of (1R,2R)-1-((2R,3R,4S,6S)-3-acetamido-4-acetoxy-6-(methoxycarbonyl)-6-(2-oxoethyl)tetrahydro-2H-pyran-2-yl)-3-(4-acetoxy-3,5-dimethylbenzamido)propane-1,2-diyl diacetate (18, 540 mg, 0.812 mmol) and 9-benzyl 2-(tert-butyl) 2,6,9-triazaspiro[4.5]decane-2,9-dicarboxylate (381 mg, 1.016 mmol) in 1,2-DCE (6 mL) at room temperature was added acetic acid (326 μL, 5.687 mmol) and activated 4 Å molecular sieves (1 g). The suspension was stirred for 2 h, after which time added sodium triacetoxyborohydride (344 mg, 1.625 mmol), and stirred for an additional 2 h. The completed reaction was diluted with EtOAc (10 mL) and sat NaHCO₃ (10 mL), filtered over a plug of Celite (1 g) eluting with EtOAc (2×5 mL ea). The layers were separated, and the organic layer was washed with brine (5 mL), dried over Na₂SO₄, filtered and concentrated. The residue was purified over a Biotage SNAP Ultra silica gel column (25 g) eluting with a 10 CV gradient of 0 to 100% EtOAc in heptane, and then 3 CV of 10% MeOH in EtOAc to provide the diastereomeric mixture (830 mg, 0.810 mmol, 100%) after combining the desired fractions, concentration, and drying under vacuum. The resulting mixture was purified by chiral HPLC to provide compound 301 (320 mg, 0.312 mmol, 39%) and 302 (260 mg, 0.254 mmol, 31%) (MW_{Calc}+H=1024.47; MW_{Obs}=1024.16) after combining the desired fractions, concentration, and drying under vacuum.

To a stirred solution of 9-benzyl 2-(tert-butyl) (S)-6-(2-((2R,4S,5R,6R)-5-acetamido-4-acetoxy-6-((1R,2R)-1,2-diacetoxy-3-(4-acetoxy-3,5-dimethylbenzamido)propyl)-2-(methoxycarbonyl)tetrahydro-2H-pyran-2-yl)ethyl)-2,6,9-triazaspiro[4.5]decane-2,9-dicarboxylate (301, 31 mg, 0.03 mmol) in IPA (1.085 mL) at room temperature was added 10% Pd—C(12.89 mg, 0.121 mmol), purged with a H₂ atmosphere (5×) followed by placing under a H₂ atmosphere (balloon pressure). The reaction mixture was stirred for 6 h, after which time the mixture was purged with a N₂ atmosphere (5×) diluted with IPA, filtered over a pad of Celite (2 g), rinsed with IPA (3×3 mL ea), and concentrated to dry. The residue was diluted with THF (620 μL) at room temperature, followed by the addition of a solution of LiOH (10.87 mg, 0.454 mmol) in water (620 μL). The reaction mixture was stirred for 16 h, after which time it was neutralized with 1 N HCl (394 μL, 0.394 mmol) being careful to keep neutral or slightly basic. The mixture was concentrated, dissolved in water (0.5 mL), and purified directly over a Sep-Pak C18 (1 g) eluting with a step gradient of 0 to 100% MeOH in Water, at 20% increments (5 mL/increment). The product containing fractions were concentrated and dried under vacuum to provide compound 303 (16 mg, 0.023 mmol, 75%) (MW_{Calc}+H=708.37; MW_{Obs}=708.29), which was used in the next step without further purification.

To a stirred solution of sodium (R)-2,3-bis(stearoyloxy)propyl (137-((2,5-dioxopyrrolidin-1-yl)oxy)-4,137-dioxo-6,9,12,15,18,21,24,27,30,33,36,39,42,45,48,51,54,57,60,63,66,69,72,75,78,81,84,87,90,93,96,99,102,105, 108,111,114,117,120,123,126,129,132,135-tetratetracontaoxa-3-azaheptatriacontahectyl) phosphate (35.6 mg, 0.012 mmol) in DMF (100 μL) at room temperature under a N2 atmosphere was added a solution of (2R,4S,5R,6R)-5-acetamido-2-(2-((R)-2-(tert-butoxycarbonyl)-2,6,9-triazaspiro[4.5]decan-6-yl)ethyl)-6-((1R,2R)-1,2-dihydroxy-3-(4-hydroxy-3,5-dimethylbenzamido)propyl)-4-hydroxytetrahydro-2H-pyran-2-carboxylic acid (303, 7.0 mg, 0.010 mmol, previously azeotroped to dry with DMF (2×0.5 mL), and placed under high vacuum for 4 h) in DMF (200 μL) followed by diisopropylethylamine (3.45 μl, 0.02 mmol). The reaction mixture was stirred for 3 h, after which time the mixture was concentrated to dry, dissolved in water (1 mL), and carefully concentrated to dry. The resultant residue was dissolve in chloroform (1 mL) and filtered through a 0.2 um filter eluting with chloroform (2×0.5 mL ea). The solution was concentrated to dry followed by diluting with water (1 mL) and purifying over a Sep-PAK C18 (1 g) eluting with a step gradient of methanol in water (10%, 25%, 50%, 75%, 90%, 100% at 10 mL each step) to provide A-373 (28 mg, 0.008 mmol, 82%) as the sodium salt after collecting the desired fractions, concentrating, and drying under vacuum.

### Preparation of A-374

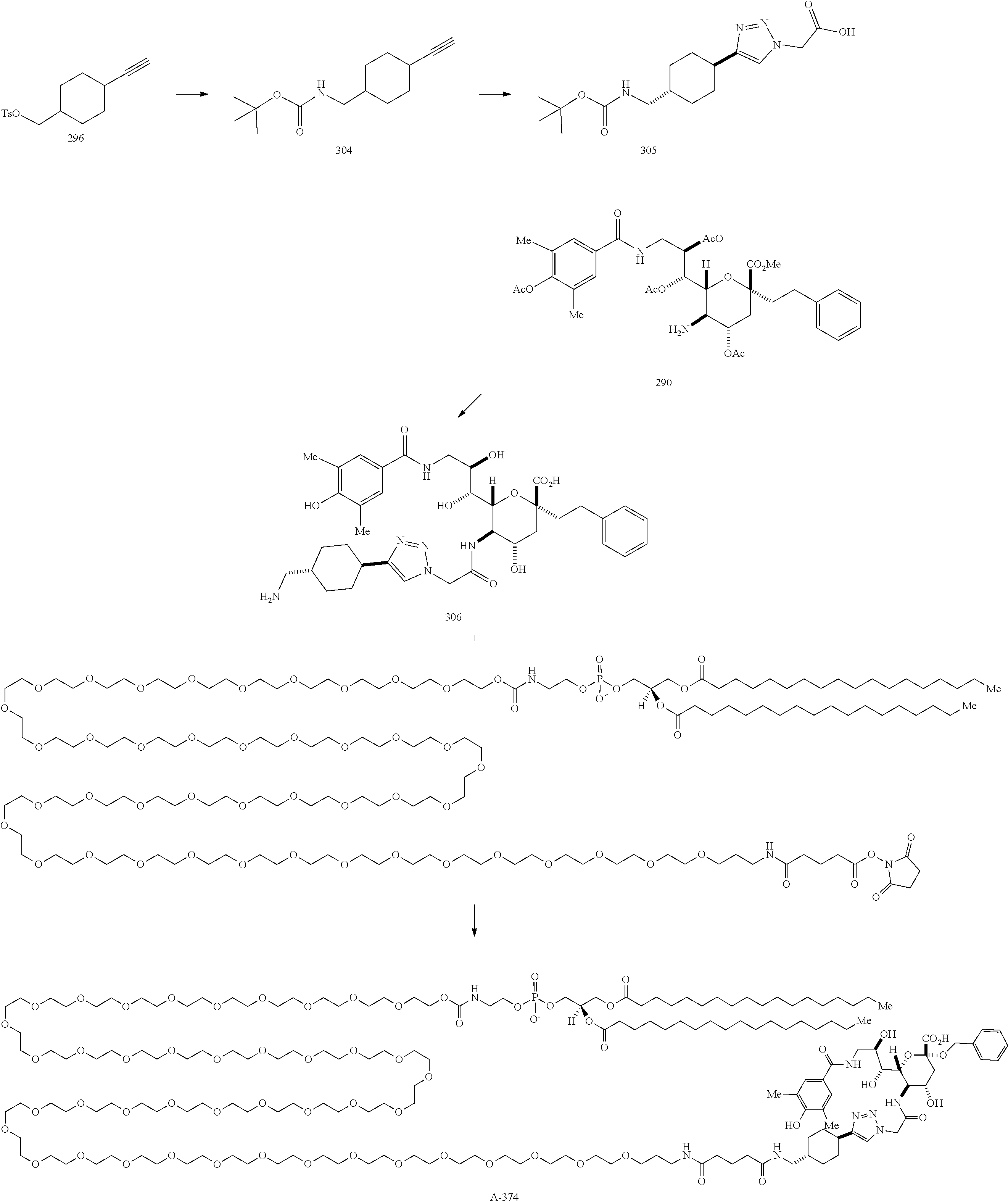

To a stirred solution of (4-ethynylcyclohexyl)methyl 4-methylbenzenesulfonate (2.10 g, 7.182 mmol) in DMF (5 mL) at room temperature was added sodium azide (296, 1.401 g, 21.546 mmol). The reaction mixture was warmed to 60° C. and stirred for 16 h. The completed reaction was cooled to room temperature, poured into a solution of 1:1 sat. NaHCO₃ in water, and the resultant mixture was extracted with Et20 (3×20 mL ea). The combined organic layers were dried over Na₂SO₄, filtered and concentrated to provide the desired azide (1.17 g, 7.17 mmol, 100%).

To a stirred solution of 1-(azidomethyl)-4-ethynylcyclohexane (1.17 g, 7.168 mmol) in THF (10 mL) at 0° C. was added 1 M trimethylphosphine in THF (10.75 mL, 10.752 mmol). The reaction mixture was warmed to room temperature, stirred for 10 min, and then concentrated. The residue was dissolved in THF (10 mL) at room temperature followed by the addition of Hunig's base (2.504 mL, 14.337 mmol), and then BOC-anhydride (2.128 mL, 9.164 mmol). The final reaction mixture was stirred for 30 min, after which time the mixture was quenched with sat. NaHCO₃ (10 mL), stirred for 1 h, and then extracted with EtOAc (3×10 mL ea). The combined organic layers were dried over Na₂SO₄, filtered, and concentrated. The resultant residue was purified over Biotage SNAP Ultra silica gel column (50 g) eluting with 6 CVs of 10% EtOAc in heptane to provide compound 304 (1.57 g, 6.62 mmol, 92%) (MW_{Calc}+H=238.17; MW_{Obs}=238.19) after combining the desired fractions, concentration, and drying under vacuum.

To a stirred solution of tert-butyl ((4-ethynylcyclohexyl)methyl)carbamate (304, 1.57 g, 6.615 mmol) in THF (20 mL) at room temperature under a N₂ atmosphere was added a solution of 25% ethyl 2-azidoacetate in toluene (3.42 g, 6.615 mmol) after which time the mixture was degassed by bubbling N₂ gas for 10 min. Sodium ascorbate (0.5 g, 2.52 mmol) and copper(i) iodide (0.252 g, 1.323 mmol) were added to the mixture followed by ethanol (15 mL), acetonitrile (10 mL) and water (15 mL) (all solvents were previously degassed with N2 gas prior to addition to the reaction mixture). Since the reaction was found to be incomplete copper(JJ) sulfate (0.528 g, 3.308 mmol) and sodium ascorbate (1.0 g, 5.04 mmol) were added, and the reaction mixture was stirred for 16 h. The suspension mixture was filtered through Celite (5 g) eluting EtOAc (3×10 mL ea). The combined filtrates were diluted with sat. NH₄Cl (20 mL) and brine (10 mL), the layers separated, and the aqueous layer was extracted with EtOAc (4×20 mL ea). The combined organic layers were dried over Na₂SO₄, filtered, and concentrated to provide the desired ester (1.57 g, 4.28 mmol, 65%) (MW_{Calc}+H=367.23; MW_{Obs}=367.27) after combining the desired fractions, concentration, and drying under vacuum.

To a stirred solution of ethyl 2-(4-((1r,4r)-4-(((tert-butoxycarbonyl)amino)methyl)cyclohexyl)-1H-1,2,3-triazol-1-yl)acetate (1.57 g, 4.284 mmol) in THF (5 mL) and MeOH (5 mL) at room temperature was added 1 N NaOH (8.57 mL, 8.569 mmol). The reaction mixture was stirred for 1 h, after which time the mixture was acidified with 1 N HCl (7.71 mL, 7.712 mmol) to pH 4, and then concentrated to remove organic solvents. The resultant precipitate was diluted with water (10 mL), filtered, and the filter pad was washed with water (2×5 mL ea). The white solid was collected and dried under vacuum to provide compound 305 (1.35 g, 3.99 mmol, 93%) (MW_{Calc}+H=339.20; MW_{Obs}=339.29) as a 10:1 mixture of the desired 1r, 4r- to Is, 4r-diastereomers.

To a stirred solution of 2-(4-((1r,4r)-4-(((tert-butoxycarbonyl)amino)methyl)cyclohexyl)-1H-1,2,3-triazol-1-yl)acetic acid (305, 133 mg, 0.393 mmol) and (1R,2R)-3-(4-acetoxy-3,5-dimethylbenzamido)-1-((2R,3R,4S,6R)-4-acetoxy-3-amino-6-(benzyloxy)-6-(methoxycarbonyl)tetrahydro-2H-pyran-2-yl)propane-1,2-diyl diacetate (290, 180 mg, 0.262 mmol) in DCM (3 mL) at room temperature were added HATU (149 mg, 0.393 mmol) and Hunig's base (0.092 mL, 0.524 mmol). The reaction mixture was stirred for 16 h, after which time the mixture was concentrated and purified directly over a Biotage SNAP Ultra silica gel column (25 g) eluting with a 10 CV gradient of 70 to 90% EtOAc in heptane to provide desired protected product (192 mg, 0.191 mmol, 73%) (MW_{Calc}+H=1008.45; MW_{Obs}=1008.43) after combining the desired fractions, concentration, and drying under vacuum.

To a stirred solution of (1R,2R)-3-(4-acetoxy-3,5-dimethylbenzamido)-1-((2R,3R,4S,6R)-4-acetoxy-6-(benzyloxy)-3-(2-(4-((1r, 4R)-4-(((tert-butoxycarbonyl)amino)methyl)cyclohexyl)-1H-1,2,3-triazol-1-yl)acetamido)-6-(methoxycarbonyl)tetrahydro-2H-pyran-2-yl)propane-1,2-diyl diacetate (215 mg, 0.213 mmol) in MeOH (3 mL) at room temperature was added 1 N NaOH (2.0 mL, 2.00 mmol). The reaction mixture was stirred for 2 h, after which time the mixture was neutralized with 1 N HCl (2.0 mL, 2.00 mmol), and concentrated. The residue was purified directly by HPLC to provide the desired acid (133.0 mg, 0.161 mmol, 76%) (MW_{Calc}+H=825.40; MW_{Obs}=825.49) after combining the desired fractions, concentration, and drying under vacuum.

To a stirred solution of (2R,4S,5R,6R)-2-(benzyloxy)-5-(2-(4-((1r,4R)-4-(((tert-butoxycarbonyl)amino)-methyl)cyclohexyl)-1H-1,2,3-triazol-1-yl)acetamido)-6-((R,2R)-1,2-dihydroxy-3-(4-hydroxy-3,5-dimethylbenzamido)propyl)-4-hydroxytetrahydro-2H-pyran-2-carboxylic acid (23.5 mg, 0.028 mmol) at room temperature with stirring was added TFA (2 mL, 25.96 mmol). The reaction mixture was stirred for 5 min, after which time it was concentrated and azeotroped to dry with acetonitrile (2 mL), toluene (3 mL), the acetonitrile (2 mL) with sonication of the suspension after each process. The resultant white solid was dissolved in MeOH (1 mL) and purified by HPLC to provide compound 306 (15.0 mg, 0.019 mmol, 68%) (MW_{Calc}+H=725.34; MW_{Obs}=725.38) as a white solid after combining the desired fractions, concentration, and drying under vacuum.

To a stirred solution of (2R,4S,5R,6R)-5-(2-(4-((r,4r)-4-(aminomethyl)cyclohexyl)-1H-1,2,3-triazol-1-yl)acetamido)-2-(benzyloxy)-6-((R,22R)-1,2-dihydroxy-3-(4-hydroxy-3,5-dimethylbenzamido)propyl)-4-hydroxytetrahydro-2H-pyran-2-carboxylic acid as a formate salt (306, 14.0 mg, 0.018 mmol) in DMSO (2 mL) at room temperature was added Hunig's base (400 μL, 2.29 mmol) 28.183 mmol) was added dropwise a solution of sodium (R)-2,3-bis(stearoyloxy)propyl (146-((2,5-dioxopyrrolidin-1-yl)oxy)-4,142,146-trioxo-5,8,11,14,17,20,23,26,29,32,35,38,41,44,47,50,53,56,59,62,65,68,71,74, 77,80,83,86,89,92,95,98,101,104,107,110,113,116,119,122,125,128,131,134,137-pentatetracontaoxa-3,141-diazahexatetracontahectyl) phosphate (45.0 mg, 0.015 mmol) in DCM (2 mL) over 7 min. The reaction mixture for 8 h, concentrated to a DMSO solution, and stirred for 16 h. The completed reaction was diluted in water (5 mL), sonication to a homogeneous suspension, and lyophilized until obtained a dry solid. The lyophilization process was repeated two additional times to obtain a white solid. The solid was dissolved in water (8 mL), placed in a dialysis tubing bag (Sigma-Aldrich D2272: 2000 MWCO), and the resultant bag was placed in stirring distilled water (4 L) for 8 h. The dialyzation process was repeated 6 times for 8 h each. The final dialyzed solution was lyophilized under high vacuum to provide A-374 (47 mg, 0.013 mmol, 87%) as a white solid.

### Analogs for Conjugation and Examples

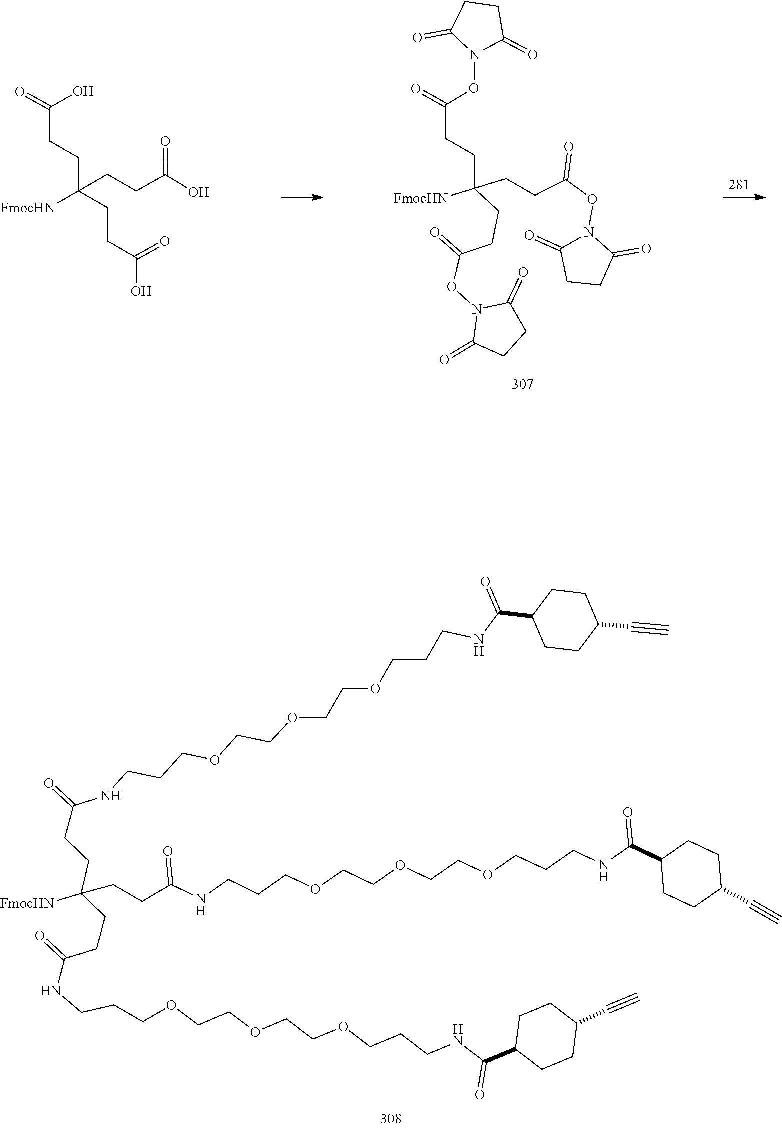

To a stirred solution of commercially available 4-((((9H-fluoren-9-yl)methoxy)carbonyl)amino)-4-(2-carboxyethyl)heptanedioic acid (1.00 g, 1.99 mmol) in DCM (16.34 mL) and DMF (3.93 mL) at room temperature was added N-hydroxysuccinimide (0.292 g, 2.539 mmol) and followed by DCC (0.454 g, 2.201 mmol). The reaction mixture was stirred at room temperature for 3 h, after which time the completed reaction was diluted with TBME (20 mL) and filtered over a pad of Celite (5 g) eluting with TBME (3×5 mL ea). The filtrate was concentrated to dryness to provide crude 307 that was used in the next reaction without further purification.

To a stirred solution of crude bis(2,5-dioxopyrrolidin-1-yl) 4-((((9H-fluoren-9-yl)methoxy)-carbonyl)amino)-4-(3-((2,5-dioxopyrrolidin-1-yl)oxy)-3-oxopropyl)heptanedioate (307, 151 mg, 0.199 mmol) in DMF (3.0 mL) at room temperature was added (1R,4R)—N-(3-(2-(2-(3-aminopropoxy)ethoxy)ethoxy)propyl)-4-ethynylcyclohexane-1-carboxamide bis(2,2,2-trifluoroacetate) (281, 244 mg, 0.698 mmol) followed by the addition of TEA (0.278 mL, 1.995 mmol). The final reaction mixture was stirred at room temperature for 16 h, after which time it was concentrated, and purified directly over a Biotage Ultra SNAP column (10 g) eluting with a gradient of 2 to 7.5% MeOH in DCM (6 CV), and then 7.5% MeOH in DCM (3 CV) to provide compound 308 (0.175 mmol, 88% yield) (MW_{Calc}+H=1478.90; MW_{Obs}=1478.80) as a colorless oil after combining the desired fractions, concentration, and drying under vacuum.

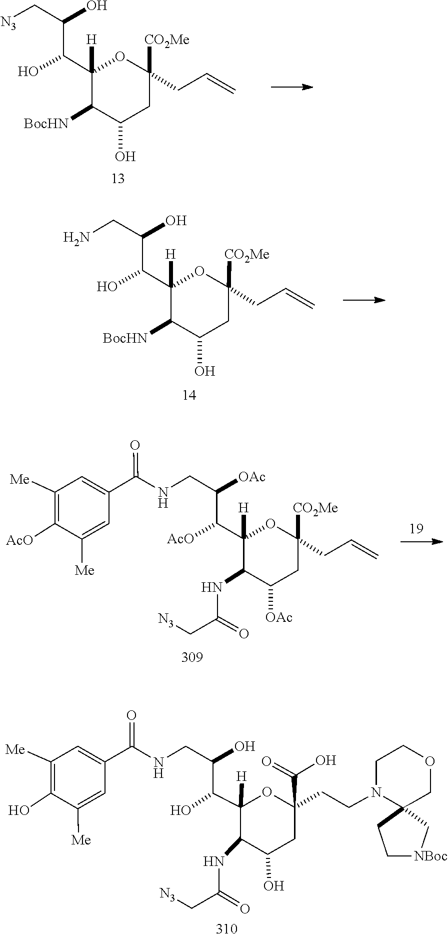 [SMILES: C=CC[C@]1(C(=O)OC)C[C@H](O)[C@@H](NC(=O)OC(C)(C)C)[C@H]([C@H](O)[C@H](O)CN=[N+]=[N-])O1 ; C=CC[C@]1(C(=O)OC)C[C@H](O)[C@@H](NC(=O)OC(C)(C)C)[C@H]([C@H](O)[C@H](O)CN)O1 ; C=CC[C@]1(C(=O)OC)C[C@H](OC(C)=O)[C@@H](NC(=O)CN=[N+]=[N-])[C@H]([C@H](OC(C)=O)[C@@H](CNC(=O)c2cc(C)c(OC(C)=O)c(C)c2)OC(C)=O)O1 ; Cc1cc(C(=O)NC[C@@H](O)[C@@H](O)[C@@H]2O[C@@](CCN3CCOC[C@@]34CCN(C(=O)OC(C)(C)C)C4)(C(=O)O)C[C@H](O)[C@H]2NC(=O)CN=[N+]=[N-])cc(C)c1O]

To a stirred solution of methyl (2R,4S,5R,6R)-2-allyl-6-((1R,2R)-3-azido-1,2-dihydroxypropyl)-5-((tert-butoxycarbonyl)amino)-4-hydroxytetrahydro-2H-pyran-2-carboxylate (13, 3.00 g, 6.97 mmol) in THF (74 ml) and water (5 ml) at 0° C. was added 1N trimethylphosphine (20.91 ml, 20.91 mmol) and added diethylamine (0.218 ml, 2.09 mmol). The reaction mixture was warmed to room temperature, stirred for 16 h, after which time it was concentrated under vacuum, and azeotroped to dry with toluene (2×30 mL ea). The crude product, 14, was used in the next step without further purification. (MW_{Calc}+H=405.22; MW_{Obs}=405.23).

To a stirred solution of crude methyl (2R,4S,5R,6R)-2-allyl-6-((1R,2R)-3-amino-1,2-dihydroxypropyl)-5-((tert-butoxycarbonyl)amino)-4-hydroxytetrahydro-2H-pyran-2-carboxylate (14, 2.8 g, 6.92 mmol) and 4-hydroxy-3,5-dimethylbenzoic acid (1.496 g, 9.00 mmol) in MeCN (28.0 mL) was added HOBT (0.53 g, 3.461 mmol) followed by EDC (1.725 g, 9.00 mmol) and triethylamine (3.86 ml, 27.69 mmol). The reaction mixture was stirred at room temperature for 3 h, after which time it was diluted with water (20 mL) and EtOAc (20 mL). The layers were separated, the aqueous layer was extracted with EtOAc (3×10 mL, ea), and the combined organic layers were washed with water (10 mL), brine (10 mL), dried over Na₂SO₄ (anhydrous), filtered and concentrated to dryness. The crude intermediate was purified over Biotage SNAP column (50 g) eluting with 10 to 100% gradient of acetone in heptane (10 CV) to provide after combining and concentration of the desired fractions to dryness the desired intermediate.

To a stirred solution of the crude intermediate above in DCM (28.0 mL) was added 1,1,1,3,3,3-Hexafluoro-2-propanol (14.00 ml, 0.135 mmol), and TMS-C1 (4.42 ml, 34.614 mmol) at room temperature. The mixture was stirred at room temperature for 2 h, after which time it was concentrated, and azeotroped to dry with MeOH (2×10 mL ea) and acetonitrile (2×10 mL ea). The protected intermediate was diluted with DCM (8.40 mL) followed by the addition of triethylamine (3.86 ml, 27.691 mmol) and then commercially available 2,5-dioxopyrrolidin-1-yl 2-azidoacetate (2.74 g, 13.846 mmol) at room temperature. The reaction mixture was stirred at room temperature for 2 h, after which time saturated NaHCO₃ (10 mL) was added followed by extraction of the mixture with EtOAc (3×10 mL ea). The combined organic layers were dried over Na₂SO₄, filtered, and concentrated to dry. The resulting residue was diluted with MeOH (20 mL) followed by the addition of Na₂CO₃ (500 mg) and stirring for 16 h at room temperature. The completed reaction was diluted with EtOAc (40 mL) and aq. NaHCO₃ and extracted with EtOAc (2×20 mL ea) and the combined organic layers were dried over Na₂SO₄ (anhydrous), filtered and concentrated to dryness. The crude intermediate was purified over a Biotage SNAP column (25 g) eluting with 10 to 100% gradient of ethyl acetate in heptane (10 CV) to provide the desired amide intermediate after combining and concentration of the desired fractions to dryness.

To a stirred solution of the amide above in DCM (5 mL) at room temperature was added pyridine (42.0 mL) followed by DMAP (0.085 g, 0.69 mmol) and acetic anhydride (6.53 mL, 69.23 mmol). The reaction mixture was stirred at room temperature for 16 h, after which time it was concentrated and purified over a Biotage SNAP column (25 g) eluting with 20 to 100% gradient of ethyl acetate in heptane (10 CV) to provide 309 (1.4 g, 2.0 mmol, 28.7% yield (5 steps)) (MW_{Calc}+H=704.28; MW_{Obs}=704.40) after combining and concentration of the desired fractions to dryness.

To a stirred solution of (1R,2R)-3-(4-acetoxy-3,5-dimethylbenzamido)-1-((2R,3R,4S,6R)-4-acetoxy-6-allyl-3-(2-azidoacetamido)-6-(methoxycarbonyl)tetrahydro-2H-pyran-2-yl)propane-1,2-diyl diacetate (309, 1.4 g, 1.989 mmol) in 1,4-dioxane (25.2 mL) and water (8.40 mL) at room temperature was added 2,6-lutidine (0.463 ml, 3.979 mmol), osmium tetroxide (0.253 ml, 0.04 mmol), and sodium periodate (1.702 g, 7.958 mmol). The reaction mixture was stirred at room temperature for 2 h, after which time it was diluted with EtOAC (50 mL) followed by saturated NaHCO₃ (15 mL) and 5% Na₂S₂O₃ (10 mL) with stirring for an additional 10 min. The resulting mixture was extracted with EtOAc (2×50 mL ea) and the combined organic layers were dried over Na₂SO₄, filtered over a pad of silica gel (10 g) eluting with EtOAc (20 mL), and concentrated to dryness. The crude aldehyde was dissolved in DCE (21.0 mL) and MeOH (4.2 mL) at room temperature followed by the addition of tert-butyl (S)-9-oxa-2,6-diazaspiro[4.5]decane-2-carboxylate (19, 0.53 g, 2.19 mmol) as a complex with (S)-mandelic acid and AcOH (0.797 mL, 13.926 mmol) and oven dried 4 Å molecular sieves (3 g). The suspension was stirred at room temperature for 1 h, after which time sodium triacetoxyborohydride (1.687 g, 7.958 mmol) was added and then stirred for 16 h. The completed reaction was quenched with saturated NaHCO₃ (10 mL), extracted with EtOAc (3×15 mL ea), and the combined organic layers were dried over Na₂SO₄ (anhydrous), filtered and concentrated to dryness. The residue was purified over a Biotage SNAP column (25 g) eluting with 20 to 100% gradient of ethyl acetate in heptane (10 CV) followed by a 0 to 30% gradient of MeOH in DCM to provide the fully protected target molecule (370 mg, 0.52 mmol, 27% yield) (MW_{Calc}+H=932.42; MW_{Obs}=932.19) after combining and concentration of the desired fractions to dryness.

To stirred solution of (1R,2R)-3-(4-acetoxy-3,5-dimethylbenzamido)-1-((2R,3R,4S,6R)-4-acetoxy-3-(2-azidoacetamido)-6-(2-((S)-2-(tert-butoxycarbonyl)-9-oxa-2,6-diazaspiro[4.5]decan-6-yl)ethyl)-6-(methoxycarbonyl)tetrahydro-2H-pyran-2-yl)propane-1,2-diyl diacetate (130 mg, 0.139 mmol) in MeOH (1.56 mL) at 0° C. was added 1 N NaOH (0.98 mL, 0.98 mmol). The reaction mixture was warmed to room temperature and stirred for 8 h, after which time 1 N NaOH (0.098 mL, 0.098 mmol) was added the mixture was stirred for 16 h. 1 N NaOH (0.098 mL, 0.098 mmol) was added the mixture was stirred for 8 h, after which time 1 N NaOH (0.098 mL, 0.098 mmol) was added the mixture was stirred for 16 h. The completed reaction was eluted directly over a Sep-Pak C18 cartridge (1 g) eluting with water (1 CV) followed by a step gradient of at 10% intervals from 10-100% MeOH in water (1 CV ea). The fractions containing the desired product was concentrated, azeotroped to dry with toluene (3×10 mL ea), followed by drying on vacuum to provide 310 (100 mg, 0.133 mmol, 96% overall yield) (MW_{Calc}-H=748784.36; MW_{Obs}=748=784.66).

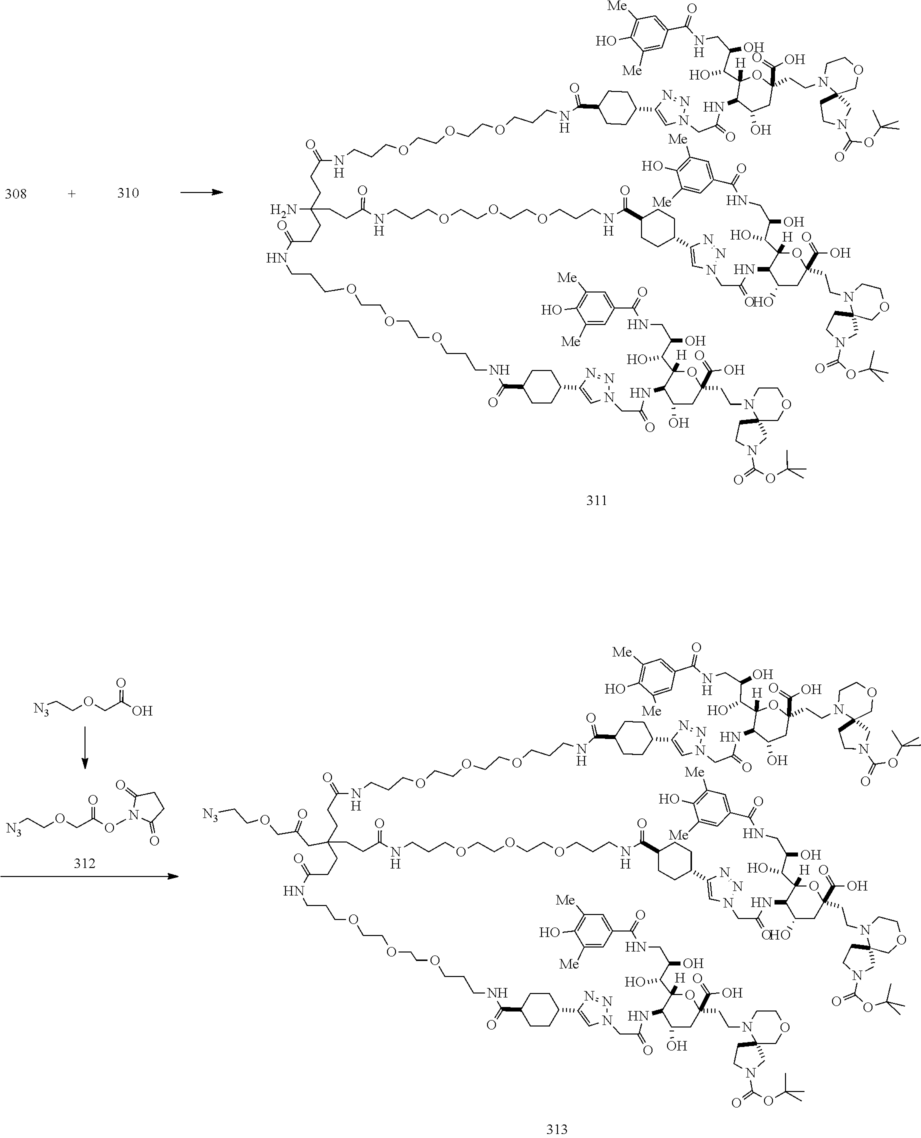

To a stirred suspension of copper iodide (5.08 mg, 0.027 mmol) in DMF (0.85 mL) at room temperature under anhydrous conditions was added tris((1-benzyl-1H-1,2,3-triazol-4-yl)methyl)amine (28.3 mg, 0.053 mmol). The mixture was stirred until a clear yellowish solution. Separately, to a stirred solution of (9H-fluoren-9-yl)methyl (1,39-bis((1r, 4r)-4-ethynylcyclohexyl)-20-(1-((1r, 4r)-4-ethynylcyclohexyl)-1,17-dioxo-6,9,12-trioxa-2,16-diazanonadecan-19-yl)-1,17,23,39-tetraoxo-6,9,12,28,31,34-hexaoxa-2,16,24,38-tetraazanonatriacontan-20-yl)carbamate (308, 59.2 mg, 0.040 mmol) in DMF (1.70 mL) at room temperature was added (2R,4S′,5R,6R)-5-(2-azidoacetamido)-2-(2-((S)-2-(tert-butoxycarbonyl)-9-oxa-2,6-diazaspiro[4.5]decan-6-yl)ethyl)-6-((1R,2R)-1,2-dihydroxy-3-(4-hydroxy-3,5-dimethylbenzamido)propyl)-4-hydroxytetrahydro-2H-pyran-2-carboxylic acid (310, 100 mg, 0.133 mmol) followed by a dropwise addition of the previous copper iodide solution over a two-minute period maintaining anhydrous conditions. The final reaction mixture was stirred at room temperature for 16 h, after which time the mixture was concentrated to approximately ¼^{th} volume, diluted with methanol (1.50 mL) with stirring at room temperature followed by 1 N NaOH (2.27 mL, 2.27 mmol). The reaction mixture was stirred for 30 minutes after which time the mixture was purified by RP HPLC (water/MeCN gradient containing 0.1% NH₄OH) to provide compound 311 (20 mg, 0.0054 mmol, 13%) (MW_{Calc}+H)/2=1815.48 MW_{Obs}=1815.22) after the desired fractions were combined, concentrated and dried under vacuum.

To a stirred solution of 2-(2-azidoethoxy)acetic acid (2.00 g, 13.78 mmol) in a mixture of DMF (8 mL) and DCM (40 mL) at room temperature was added hydroxysuccinimide (2.38 g, 20.67 mmol) followed by the addition of N,N′-dicyclohexylcarbodiimide (5.68 g, 27.53 mmol). The reaction mixture was stirred at room temperature for 16 h, after which time the completed reaction was diluted with TBME (30 mL) followed by filtration through a pad of Celite (5 g) eluting with TBME (3×5 mL ea). The filtrate was concentrated, diluted with water (20 mL) and extracted with EA (3×30 mL ea). The combined organic layers were washed with water (50 mL), brine (50 mL), dried over Na₂SO₄, filtered and concentrated. The residue was purified Biotage SNAP column (100 g) eluting with 10 to 50% gradient of ethyl acetate in petroleum ether (10 CV) by flash column chromatography (silica gel, eluting with 10%˜50% EA/heptane) to give compound 312 (2.00 g, 8.26 mmol, 60.0%) as a white solid. ¹H NMR: (400 MHz, CDCl3) δ 4.51 (s, 2H), 3.81 (t, J=4.8 Hz, 2H), 3.47 (t, J=4.8 Hz, 2H), 2.87 (s, 4H).

To stirred solution of compound 311 (10 mg, 0.0028 mmol) and 2,5-dioxopyrrolidin-1-yl 2-(2-azidoethoxy)acetate (312, 3.5 mg, 0.014 mmol) in DMF (0.1 ml) at room temperature was added triethylamine (5.5 mg, 0.029 mmol). The reaction mixture was stirred at room temperature for 24 h, after which time the completed reaction mixture was loaded directly onto a C18 cartridge (1 g) and eluting with a 10 to 70% gradient of CH₃CN in water (5 CV). The fractions containing products was concentrated and purified by RP HPLC (water/MeCN gradient containing 0.1% NH₄OH) followed by the 2^{nd} RP HPLC (water/MeCN gradient containing 0.1% formic acid) to provided compound 313 (2.3 mg, 0.00063 mmol, 22%) as a yellow solid. (MW_{Calc}/2+H=1815.98; MW_{Obs}=1815.22)

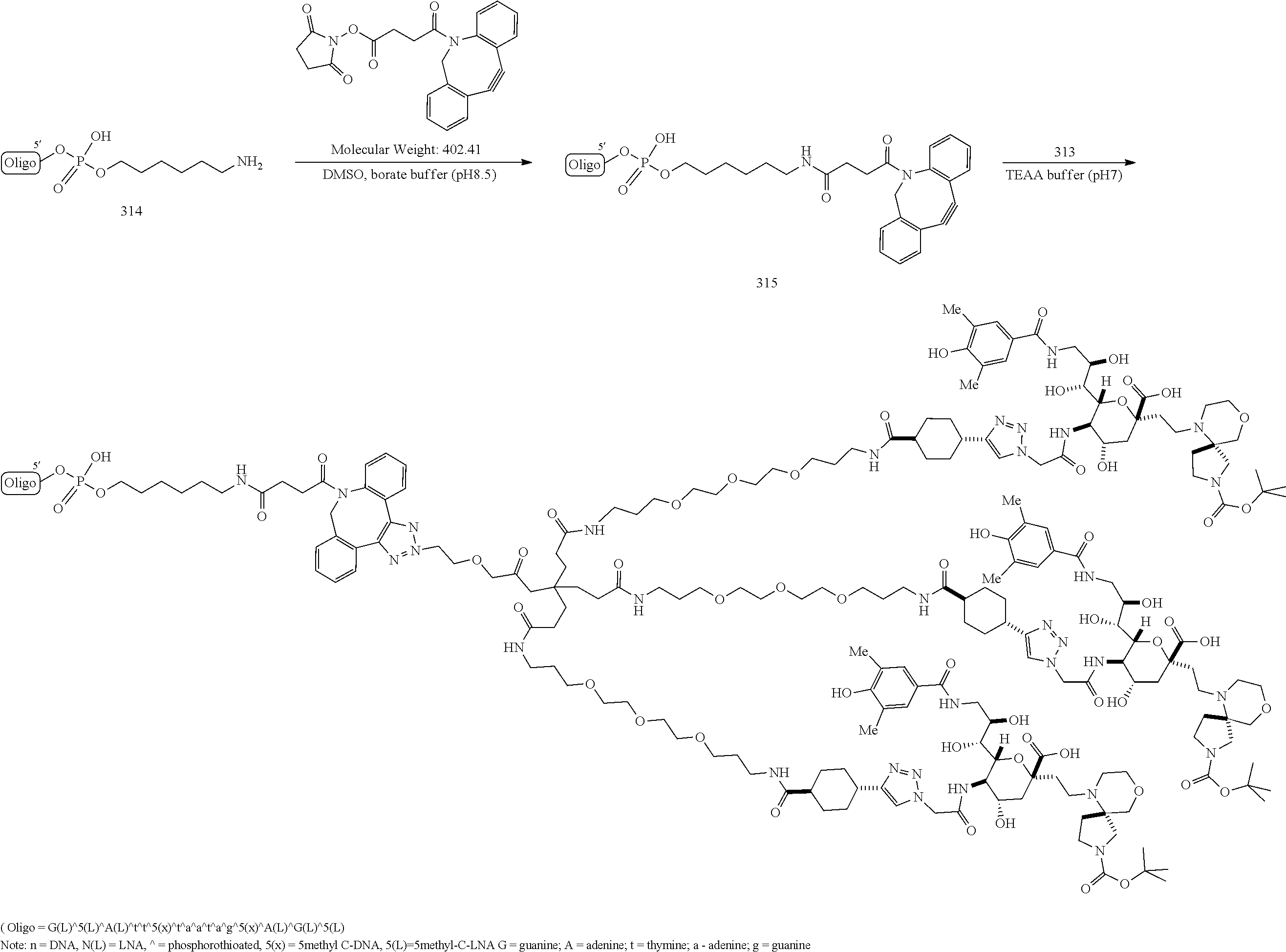

To a stirred solution of commercially available compound 314 (10 mg, 0.0016 mmol) in 0.5 M borate buffer (0.12 mL, pH 8.5) was added at room temperature commercially available dibenzocyclooctyne-N-hydroxysuccinimidyl ester (20.9 mg, 0.052 mmol) in DMSO (0.52 mL). The mixture was stirred for 30 minutes, after which time it was filtered, and washed repeatedly with water using ultrafiltration (VIVASPIN 20, 3K) to provided compound 315 (MW 6703.5, found 6703.2; LC-MS).

To a suspension of 315 (2.2 mg, 0.330 μmol) in water (400 μL) at room temperature was added 1M triethylammonium acetate buffer (40 μL, pH7) and 313 (2.18 mg, 0.60 mmol) in DMSO (120 μL) followed by mixing for 1 hour. The resultant completed reaction was washed with water by ultrafiltration (VIVASPIN 20, 3K), and further purified over a reverse phase HPLC. The desired collected fraction was evaporated, dissolved with 1 M sodium acetate, repeatedly washed with water by ultrafiltration (VIVASPIN 20, 3K), and evaporated to dryness to provide compound 316 (1.6 mg, 0.154 μmol, 47%) as white powder. (MW_{Calc}+H=10336.6; MW_{Obs}=10336.6).

Compound 317 was prepared in a similar way to compound 316 starting with commercially available 5′-N(6)-G(L)\hat{ }G(L)\hat{ }5(L)\hat{ }t\hat{ }a\hat{ }5(x)\hat{ }t\hat{ }a\hat{ }5(x)\hat{ }g\hat{ }5(x)\hat{ }5(x)\hat{ }g\hat{ }T(L)\hat{ }5(L)\hat{ }A(L)-Cy3 (10 mg) instead of compound 314 to provide compound 317 which was used without purification.

To a stirred solution of commercially available MALAT1 targeting gapmer G(L)\hat{ }G(L)\hat{ }5(L)\hat{ }t\hat{ }a\hat{ }5(x)\hat{ }t\hat{ }a\hat{ }5(x)\hat{ }g\hat{ }5(x)\hat{ }5(x)\hat{ }g\hat{ }T(L)\hat{ }5(L)\hat{ }A(L) 314 (10.3 mg, 0.0016 mmol) in 0.5 M borate buffer (0.12 mL, pH 8.5) was added at room temperature commercially available dibenzocyclooctyne-N-hydroxysuccinimidyl ester (20.9 mg, 0.052 mmol) in DMSO (0.52 mL). The mixture was stirred for 30 minutes, after which time it was filtered, and washed 5 times with water using ultrafiltration (VIVASPIN 20, 3K) to provided compound 315 (2.2 mg, 0.330 μmol, 21%) (MW 6703.5, found 6703.2; LC-MS). For compound 314: n=DNA, N(L)=LNA, \hat{ }=phosphorothioated, 5(x)=5 methyl C-DNA, 5(L)=5-methyl cytosine -LNA, G=guanine; A=adenine; t=thymine; α-adenine; g=guanine.

To a suspension of 315 (2.2 mg, 0.330 μmol) in water (400 μL) at room temperature was added 1M triethylammonium acetate buffer (40 μL, 40 μL, pH7) and 313 (2.18 mg, 0.60 mmol) in DMSO (120 μL) followed by mixing for 1 hour. The resultant completed reaction was washed with water by ultrafiltration (VIVASPIN 20, 3K), and further purified over a reversed-phase HPLC (XBridge BEH C18 OBD prep 10×150 mm, 5 um, column temperature 55° C.; Eluent A: 100 mM pyrogen free water, 8.6 mM TEA in water, Eluent B: MeOH 5-90% gradient over 20 minutes). The desired collected fraction was evaporated, dissolved with sodium acetate, repeatedly washed with water by ultrafiltration (VIVASPIN 20, 3K), and evaporated to dryness to provide compound 316 (1.6 mg, 0.154 μmol, 47%) as white powder. (MW_{Calc}+x=10336.6; MW_{Obs}=10336.6).

Compound 317 was prepared in a similar way to compound 316 starting with commercially available 5′-N(6)-G(L)\hat{ }G(L)\hat{ }5(L)\hat{ }t\hat{ }a\hat{ }5(x)\hat{ }t\hat{ }a\hat{ }5(x)\hat{ }g\hat{ }5(x)\hat{ }5(x)\hat{ }g\hat{ }T(L)\hat{ }5(L)\hat{ }A(L)-Cy3 (10 nmole) instead of compound 314 to provide compound 317 which was used without purification.

### Preparation of A-375

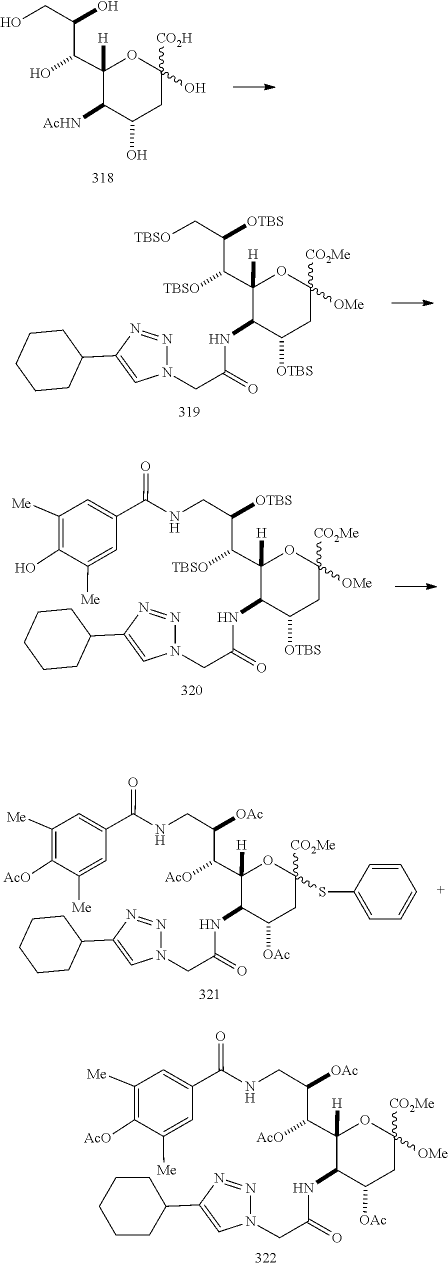

To a stirred suspension of commercially available (2R,4S,5R,6R)-5-acetamido-2,4-dihydroxy-6-((1R,2R)-1,2,3-trihydroxypropyl)tetrahydro-2H-pyran-2-carboxylic acid (318, 5.00 g, 16.167 mmol) in pyridine (30 mL) was added acetic anhydride (15 mL, 158.98 mmol) after which time the reaction mixture was warmed to 60° C. and stirred for 2 h. The completed reaction was concentrated and azeotroped with toluene (4×100 mL ea). The residue was dissolved in 4 N HCl (80 mL, 240.00 mmol) and the reddish yellow mixture was heated at reflux for 24 hours. The dark solution was cooled to room temperature, after which time it was concentrated and purified over a Biotage SNAP column (100 g) eluting with 10% to 40% methanol in dichloromethane (5 CV) and then a 1:19:30 ratio of water:methanol: dichloromethane (2 CV) to provide crude (4S,5R,6R)-methyl 5-amino-4-hydroxy-2-methoxy-6-((1R,2R)-1,2,3-trihydroxypropyl)tetrahydro-2H-pyran-2-carboxylate (5.1 g, >100%) as a dark solid after concentration of the desired fractions, azeotroping with toluene (2×50 mL ea), and high vacuum to dryness. (MW_{Calc}+H=338.14; MW_{Obs}=338.14).

To a stirred solution of (4S,5R,6R)-methyl 5-amino-4-hydroxy-2-methoxy-6-((1R,2R)-1,2,3-trihydroxypropyl)tetrahydro-2H-pyran-2-carboxylate (2.00 g, 6.773 mmol) in DMF (20 mL) at room temperature was added imidazole (9.22 g, 135.46 mmol) followed by tert-butyldimethylsilyl chloride (12.25 g, 81.28 mmol). The reaction mixture was stirred for 3 h, warmed to 60° C., and stirred for an additional 2 h. The completed reaction was cooled to room temperature, after which time it was poured over a mixture of ice water (100 mL) and 0.1 N HCl (10 mL), followed by extraction with EtOAc (3×100 mL ea). The combined organic layers were dried over Na₂SO₄, filtered and concentrated to give a dark oil. The crude product was purified twice over a Biotage SNAP column (100 g) eluting with 5% ethyl acetate in heptane (7 CV) to provide the desired (4S,5S,6R)-methyl 5-amino-4-((tert-butyldimethylsilyl)oxy)-6-((5S,6R)-6-((tert-butyldimethylsilyl)oxy)-2,2,3,3,9,9,10,10-octamethyl-4,8-dioxa-3,9-disilaundecan-5-yl)-2-methoxytetrahydro-2H-pyran-2-carboxylate (3.45 g, 4.59 mmol, 68%) after concentration of the desired fractions and drying under high vacuum. (MW_{Calc}+H=752.47; MW_{Obs}=752.48).

To a stirred suspension of (4S,5S,6R)-methyl 5-amino-4-((tert-butyldimethylsilyl)oxy)-6-((5S,6R)-6-((tert-butyldimethylsilyl)oxy)-2,2,3,3,9,9,10,10-octamethyl-4,8-dioxa-3,9-disilaundecan-5-yl)-2-methoxytetrahydro-2H-pyran-2-carboxylate (3.45 g, 4.586 mmol) in dichloromethane (30 mL) at room temperature was added 2-(4-cyclohexyl-1H-1,2,3-triazol-1-yl)acetic acid (1.343 g, 6.42 mmol), HATU (2.267 g, 5.961 mmol) in dichloromethane (30 ml, 466.252 mmol), followed by triethylamine (7.26 ml, 52.11 mmol). The reaction mixture was diluted with DMF (14.53 mL) and the resulting mixture was stirred for 40 h. The completed reaction was concentrated and purified over a Biotage SNAP column (100 g) eluting with 15% ethyl acetate in heptane (8 CV) to provide compound 319 (3.00 g, 3.18 mmol, 69%) after concentration of the desired fractions and drying under high vacuum. (MW_{Calc}+H=943.58; MW_{Obs}=943.38).

To a stirred solution of (2R,4S,5S,6R)-methyl 4-((tert-butyldimethylsilyl)oxy)-6-((5S,6R)-6-((tert-butyldimethylsilyl)oxy)-2,2,3,3,9,9,10,10-octamethyl-4,8-dioxa-3,9-disilaundecan-5-yl)-5-(2-(4-cyclohexyl-1H-1,2,3-triazol-1-yl)acetamido)-2-methoxytetrahydro-2H-pyran-2-carboxylate (319, 690 mg, 0.731 mmol) in methanol (6 mL) and dichloromethane (6 mL) at room temperature was added CSA (77 mg, 0.33 mmol). The reaction mixture was stirred for 3 h, after which time it was quenched with TEA (1.02 mL, 7.31 mmol) and concentrated. The crude product was purified over a Biotage SNAP column (25 g) eluting with a gradient of 10% to 40% ethyl acetate in heptane (10 CV) and then 40% ethyl acetate in heptane (3 CV) to provide (2R,4S,5S,6R)-methyl 4-((tert-butyldimethylsilyl)oxy)-5-(2-(4-cyclohexyl-1H-1,2,3-triazol-1-yl)acetamido)-6-((5S,6R)-6-(hydroxymethyl)-2,2,3,3,8,8,9,9-octamethyl-4,7-dioxa-3,8-disiladecan-5-yl)-2-methoxytetrahydro-2H-pyran-2-carboxylate (455 mg, 0.549 mmol, 75%) after concentration of the desired fractions and drying under high vacuum. (MW_{Calc}+H=829.49; MW_{Obs}=829.27).

To a stirred solution of (4S,5S,6R)-methyl 4-((tert-butyldimethylsilyl)oxy)-5-(2-(4-cyclohexyl-1H-1,2,3-triazol-1-yl)acetamido)-6-((5S,6R)-6-(hydroxymethyl)-2,2,3,3,8,8,9,9-octamethyl-4,7-dioxa-3,8-disiladecan-5-yl)-2-(phenylthio)tetrahydro-2H-pyran-2-carboxylate (455 mg, 0.549 mmol) in dichloromethane (2 mL) at 0° C. was added TEA (0.229 mL, 1.646 mmol) followed by methanesulfonyl chloride (0.051 mL, 0.658 mmol). The reaction mixture was stirred for 10 min, after which time the completed reaction was quenched by addition of sat. NaHCO₃ (5 mL), followed by extraction with dichloromethane (4×5 mL ea). The combined organic layers were dried over Na₂SO₄, filtered, and concentrated to dryness to provide the crude mesylate (498 mg, 0.549 mmol, 100%) as a pale yellow foamy solid. (MW_{Calc}+H=907.47; MW_{Obs}=907.16).

To a stirred suspension of the above crude mesylate (498 mg, 0.549 mmol) in DMF (5 mL) at room temperature was added sodium azide (178 mg, 2.744 mmol). The mixture was warmed to 60° C. and stirred for 24 h, after which time it was warmed to 90° C. and stirred for 16 h. The mixture was cooled to room temperature, diluted with a 1:1 mixture of brine and 0.1 M HCl (20 mL), and extracted with dichloromethane (3×10 mL ea). The combined organic layers were dried over Na₂SO₄, filtered and concentrated to give the crude product in residual DMF.

The crude mixture from above was diluted with stirring in methanol (2 mL), cooled to 0° C. followed by an addition of 2.0 M trimethylsilyldiazomethane (0.823 mL, 1.646 mmol) in methanol. The reaction mixture was stirred for 15 min, after which time it was concentrated to a DMF solution. The stirred intermediate was warmed to room temperature, and then treated with imidazole (374 mg, 5.488 mmol) followed by tert-butyldimethylsilyl chloride (331 mg, 2.195 mmol). The final reaction mixture was stirred at room temperature for 72 h followed by warming to 65° C. for 2 h. The completed reaction was concentrated and the residue was purified over a Biotage SNAP column (80 g) eluting with a gradient of 10% to 20% ethyl acetate in heptane (10 CV) and then 20% ethyl acetate in heptane (3 CV) to provide (2R,4S,5S,6R)-methyl 6-((5S,6R)-6-(azidomethyl)-2,2,3,3,8,8,9,9-octamethyl-4,7-dioxa-3,8-disiladecan-5-yl)-4-((tert-butyldimethylsilyl)oxy)-5-(2-(4-cyclohexyl-1H-1,2,3-triazol-1-yl)acetamido)-2-methoxytetrahydro-2H-pyran-2-carboxylate (375 mg, 0.439 mmol, 80%) after concentration of the desired fractions and drying under high vacuum. (MW_{Calc}+H=854.50; MW_{Obs}=854.39).

To a stirred solution of (2R,4S,5S,6R)-methyl 6-((5S,6R)-6-(azidomethyl)-2,2,3,3,8,8,9,9-octamethyl-4,7-dioxa-3,8-disiladecan-5-yl)-4-((tert-butyldimethylsilyl)oxy)-5-(2-(4-cyclohexyl-1H-1,2,3-triazol-1-yl)acetamido)-2-methoxytetrahydro-2H-pyran-2-carboxylate (1.87 g, 2.189 mmol) in THF (20 mL) and water (2 mL) at room temperature was add triphenylphosphine (2.87 g, 10.944 mmol. The reaction mixture was warmed to 65° C. for 1 h, after which time the completed reaction was cooled to room temperature and concentrated. The residue was purified over a Biotage SNAP column (80 g) eluting with a gradient of 5% to 10% methanol in dichloromethane (10 CV) and then 10% methanol in dichloromethane (3 CV) to provide (2R,4S,5S,6R)-methyl 6-((5S,6R)-6-(aminomethyl)-2,2,3,3,8,8,9,9-octamethyl-4,7-dioxa-3,8-disiladecan-5-yl)-4-((tert-butyldimethylsilyl)oxy)-5-(2-(4-cyclohexyl-1H-1,2,3-triazol-1-yl)acetamido)-2-methoxytetrahydro-2H-pyran-2-carboxylate (1.36 g, 1.642 mmol, 75%) after concentration of the desired fractions and drying under high vacuum. (MW_{Calc}+H=828.51; MW_{Obs}=828.28).

To a stirred suspension of (2R,4S,5S,6R)-methyl 6-((5S,6R)-6-(aminomethyl)-2,2,3,3,8,8,9,9-octamethyl-4,7-dioxa-3,8-disiladecan-5-yl)-4-((tert-butyldimethylsilyl)oxy)-5-(2-(4-cyclohexyl-1H-1,2,3-triazol-1-yl)acetamido)-2-methoxytetrahydro-2H-pyran-2-carboxylate (800 mg, 0.966 mmol) in dichloromethane (10 mL, 155.417 mmol) at room temperature was added 4-hydroxy-3,5-dimethylbenzoic acid (225 mg, 1.352 mmol), followed by HATU (477 mg, 1.256 mmol) and TEA (0.808 mL, 5.795 mmol). The reaction mixture was stirred for 16 h, after which time the completed reaction was concentrated and purified over a Biotage SNAP column (120 g) eluting with a gradient of 20% ethyl acetate in dichloromethane (13 CV) to provide 320 (631 mg, 0.646 mmol, 67%) after concentration of the desired fractions and drying under vacuum. (MW_{Calc}+H=977.56; MW_{Obs}=977.80).

To a stirred solution of (2R,4S,5S,6R)-methyl 4-((tert-butyldimethylsilyl)oxy)-5-(2-(4-cyclohexyl-1H-1,2,3-triazol-1-yl)acetamido)-6-((5S,6R)-6-((4-hydroxy-3,5-dimethylbenzamido)methyl)-2,2,3,3,8,8,9,9-octamethyl-4,7-dioxa-3,8-disiladecan-5-yl)-2-methoxytetrahydro-2H-pyran-2-carboxylate (320, 160 mg, 0.164 mmol) in dichloromethane (0.2 mL) at room temperature was added and thiophenol (0.337 mL, 3.277 mmol) followed by a dropwise addition of BF₃OEt₂ (0.311 mL, 2.458 mmol). The reaction was warmed to 65° C. and stirred for 4 h. The resulting light brownish yellow mixture was then cooled to 0° C., followed by a slow addition of pyridine (2.65 mL) and then acetic anhydride (1.55 mL, 16.39 mmol). The reaction was stirred for 2 h, after which time TLC (70% ethyl acetate in heptane) showed a mixture of peracetylated products. The mixture was concentrated and purified twice over a Biotage SNAP column (12 g) eluting with a gradient of 50% to 100% ethyl acetate in heptane (2 CV), 100% EtOAc (2 CV), and then 5% MeOH in EtOAc (2 CV). The desired mixture of products was concentrated to dry, followed by the addition of pyridine (2 mL) at room temperature with stirring, and then acetic anhydride (1.00 mL, 10.57 mmol). The final reaction mixture was warmed to 65° C. and stirred for 30 min. The completed reaction was cooled to room temperature, azeotroped with toluene (3×5 mL ea), and dried under vacuum. The residue was first purified over a Biotage SNAP column (25 g) eluting with 60% ethyl acetate in toluene to provide a mixture of desired products after collection and concentration to dry. The mixture was plated on four prep TLC silica plates (Merck AG) eluting with 70% ethyl acetate in toluene providing 321 (53 mg, 0.060 mmol, 37%) (MW_{Calc}+H=880.34; MW_{Obs}=880.09) and 322 (38 mg, 0.047 mmol, 29%) (MW_{Calc}+H=803.34; MW_{Obs}=803.45), after eluting each band individual and concentration to dryness.

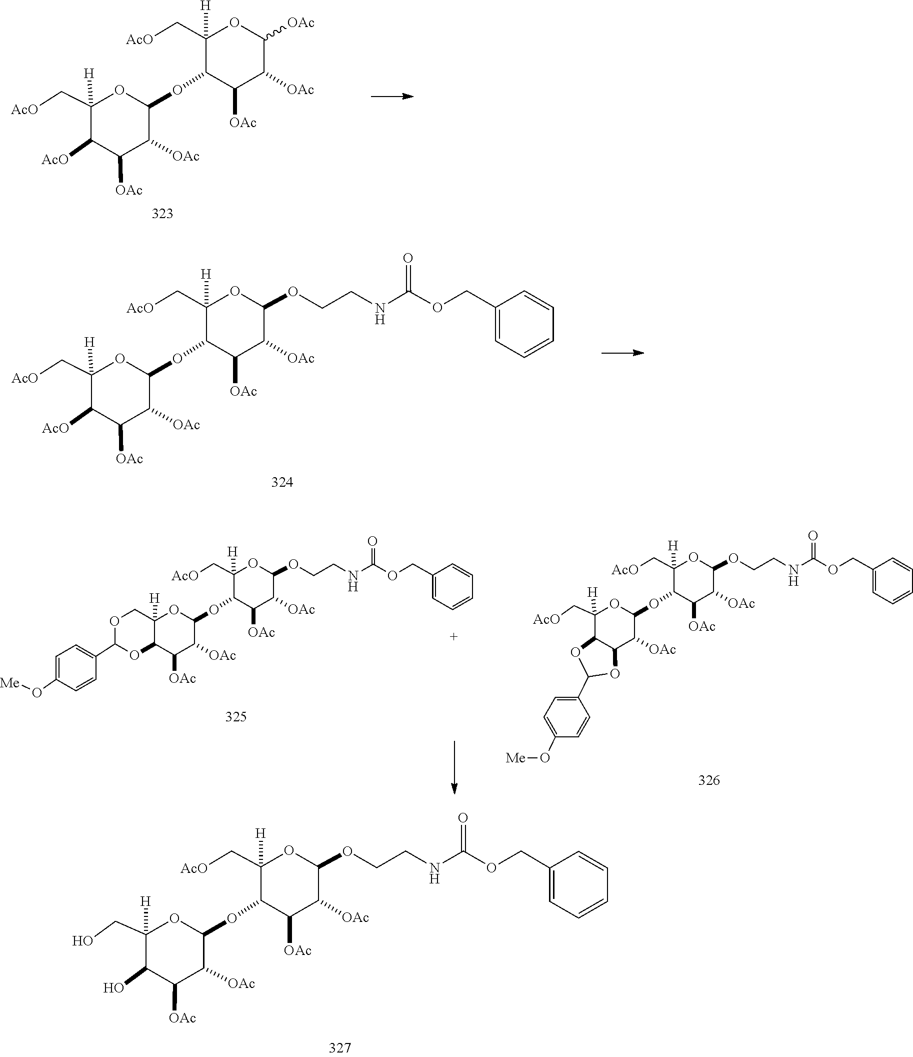

To commercially available (2S,3R,4S,5R,6R)-6-(acetoxymethyl)-5-(((2S,3R,4S,5S,6R)-3,4,5-triacetoxy-6-(acetoxymethyl)tetrahydro-2H-pyran-2-yl)oxy)tetrahydro-2H-pyran-2,3,4-triyl triacetate (323, 12.5 g, 18.421 mmol) at room temperature with stirring was slowly added 5.07 M hydrogen bromide in acetic acid solution (62.5 ml, 361.97 mmol). The reaction mixture was stirred for 30 min, after which time the completed reaction was concentrated and azeotroped to dry with toluene (3×100 mL ea). The resulting residue was dissolved in ethyl acetate (100 mL), washed with a 1:1 solution of sat. NaHCO₃/brine (2×25 mL ea), and the combined aqueous layers were back extracted with ethyl acetate (25 mL). The combined organic layers were dried over Na₂SO₄, filtered, concentrated, placed under vacuum to dry to provide the desired crude (2R,3S,4S,5R,6S)-2-(acetoxymethyl)-6-(((2R,3R,4S,5R,6S)-4,5-diacetoxy-2-(acetoxymethyl)-6-bromotetrahydro-2H-pyran-3-yl)oxy)tetrahydro-2H-pyran-3,4,5-triyl triacetate (12.88 g, 18.41 mmol, 100%) (MW_{Calc}+Na=721.11; MW_{Obs}=721.0).

To the stirred suspension of silver trifluoromethanesulfonate (2.53 g, 9.831 mmol) in dichloromethane (29.0 mL) at 0° C. was slowly added the solution of (2R,3S,4S,5R,6S)-2-(acetoxymethyl)-6-(((2R,3R,4S,5R,6S)-4,5-diacetoxy-2-(acetoxymethyl)-6-bromotetrahydro-2H-pyran-3-yl)oxy)tetrahydro-2H-pyran-3,4,5-triyl triacetate (5.73 g, 8.192 mmol) and benzyl (2-hydroxyethyl)carbamate (3.20 g, 16.384 mmol) in dichloromethane (29.0 mL) maintaining the temperature at 0° C. The reaction mixture was stirred at 0° C. for 0.5 hr, after which time it was quenched with a 1:1 mixture of sat. NaHCO₃/brine (50 mL), followed by extraction with a 1:1 ratio of ethyl acetate in heptane (3×25 mL ea). The combined organic layers were dried over Na₂SO₄, filtered, and concentrated to dry. The residues was dissolved with stirring in pyridine (6 mL) at room temperature followed by the addition of acetic anhydride (3 mL, 31.71 mmol). The reaction mixture was warmed to 57° C. and stirred for 3 hrs. The completed reaction was cooled to room temperature, concentrated, and azeotroped to dry. The residue was purified over a Biotage SNAP column (100 g) eluting with a gradient of 0 to 100% ethyl acetate in heptane (10 CV) and ethyl acetate (20 CV) to provide 324 (4.23 g, 5.20 mmol, 64%). (MW_{Calc}+H=814.27; MW_{Obs}=814.0) after concentration of the desired fractions and drying under vacuum.

To a stirred solution of (2R,3S,4S,5R,6S)-2-(acetoxymethyl)-6-(((2R,3R,4S,5R,6R)-4,5-diacetoxy-2-(acetoxymethyl)-6-(2-(((benzyloxy)carbonyl)amino)ethoxy)tetrahydro-2H-pyran-3-yl)oxy)tetrahydro-2H-pyran-3,4,5-triyl triacetate (324, 4.23 g, 5.198 mmol) in methanol (100 mL) at room temperature was added 1N NaOH (100 mL, 100.00 mmol). The reaction mixture was stirred for 3 hr, after which time the completed reaction was neutralized with 1 N HCl (100 ml, 100.00 mmol). The resulting mixture was concentrated to approximately 50 mL, and slowly mixed with silica gel (20 g), after which time the suspension was poured over a pad of silica gel (50 g), and then eluted with a 2:1 mixture of dichloromethane in methanol (5 CV). The filtrate was combined and concentrated to dryness to provide crude benzyl (2-(((2R,3R,4R,5S,6R)-3,4-dihydroxy-6-(hydroxymethyl)-5-(((2S,3R,4S,5R,6R)-3,4,5-trihydroxy-6-(hydroxymethyl)tetrahydro-2H-pyran-2-yl)oxy)tetrahydro-2H-pyran-2-yl)oxy)ethyl)carbamate (2.70 g. 5.20 mmol, 100%) (MW_{Calc}+H=520.20; MW_{Obs}=524.0).

To a stirred solution of benzyl (2-(((2R,3R,4R,5S,6R)-3,4-dihydroxy-6-(hydroxymethyl)-5-(((2S,3R,4S,5R,6R)-3,4,5-trihydroxy-6-(hydroxymethyl)tetrahydro-2H-pyran-2-yl)oxy)tetrahydro-2H-pyran-2-yl)oxy)ethyl)carbamate (3.0 g, 5.775 mmol) in DMF (60 mL) at room temperature was added 1-(dimethoxymethyl)-4-methoxybenzene (2.96 ml, 17.32 mmol) followed by p-toluenesulfonic acid monohydrate (0.038 g, 0.202 mmol). The reaction mixture was warmed to 70° C. and stirred for 1 hr. The completed intermediate reaction was diluted with pyridine (60.7 mL) was added followed by a slow addition of addition of acetic anhydride (30.0 mL, 317.62 mmol). The final reaction mixture was stirred at 70° C. for 3 hrs, cooled to room temperature and stirred for an additional 16 h. The reaction was slowly quenched up with a 1:1 mixture of sat. NaHCO₃:brine (100 mL) and extracted with ethyl acetate (4×50 mL ea). The combined organic layers were dried over Na₂SO₄, filtered, and concentrated to dry. The residue was purified over a Biotage SNAP column (100 g) eluting with a gradient of 50% to 80% ethyl acetate in heptane (10 CV), 80% ethyl acetate in heptane (10 CV), and ethyl acetate (10 CV) to provide a mixture of 325 (1.85 g, 2.18 mmol, 38%) (MW_{Calc}+H=848.27; MW_{Obs}=848.0), and 326 after separately combining the desired fractions and concentrating to dryness.

To a stirred solution of (2R,3R,4S,5R,6R)-6-(acetoxymethyl)-2-(2-(((benzyloxy)carbonyl)amino)ethoxy)-5-(((4aR,6S,7R,8S,8aS)-7,8-diacetoxy-2-(4-methoxyphenyl)hexahydropyrano[3,2-d][1,3]dioxin-6-yl)oxy)tetrahydro-2H-pyran-3,4-diyl diacetate (325, 1.7 g, 2.005 mmol) in acetonitrile (105 mL) at 0° C. was added ceric ammonium nitrate (3.30 g, 6.015 mmol) and water (10.8 mL). The reaction mixture was stirred at 0° C. for 2 hr, warmed to room temperature and stirred an additional 16 h. The reaction was quenched with triethylamine (1.01 mL, 7.219 mmol), followed by a 1:1 mixture of sat. NaHCO₃:brine (20 mL). The mixture was extracted with ethyl acetate (3×20 mL ea), and the combined organic layers were dried over Na₂SO₄, filtered, and concentrated to dry. The residue was purified over a Biotage SNAP column (50 g) eluting with a gradient of 0 to 100%, ethyl acetate in heptane (5 CV), and ethyl acetate (10 CV) to provided 327 (1.05 g, 1.439 mmol, 72%) after combining the desired fractions and concentrating to dryness. (MW_{Calc}+H=730.25; MW_{Obs}=734.0)

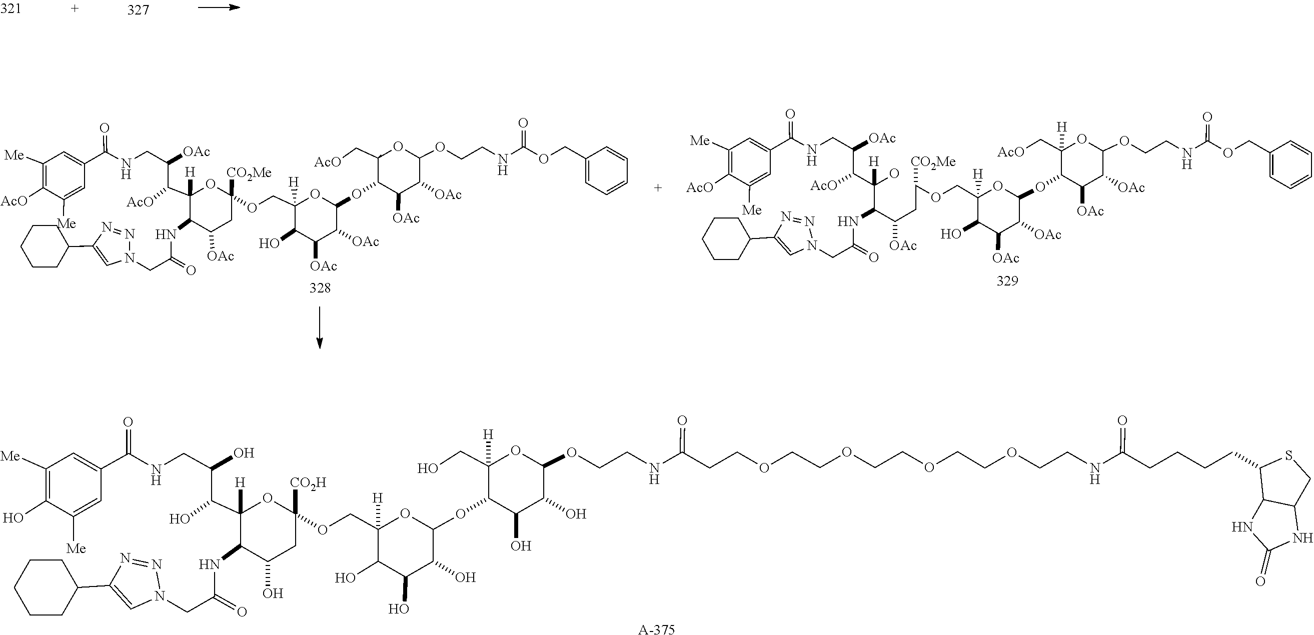

A mixture of (1R,2R)-3-(4-acetoxy-3,5-dimethylbenzamido)-1-((2R,3R,4S)-4-acetoxy-3-(2-(4-cyclohexyl-1H-1,2,3-triazol-1-yl)acetamido)-6-(methoxycarbonyl)-6-(phenylthio)tetrahydro-2H-pyran-2-yl)propane-1,2-diyl diacetate (321, 60 mg, 0.068 mmol) and (2R,3R,4S,5R,6R)-6-(acetoxymethyl)-2-(2-(((benzyloxy)carbonyl)amino)ethoxy)-5-(((2S,3R,4S,5S,6R)-3,4-diacetoxy-5-hydroxy-6-(hydroxymethyl)tetrahydro-2H-pyran-2-yl)oxy)tetrahydro-2H-pyran-3,4-diyl diacetate (327, 54.7 mg, 0.075 mmol) was azeotroped to dryness with toluene (3×5 mL ea), and then put under high vacuum with a stirring bar for 20 min. 3 A activated molecular sieves (600 mg) was added under a N₂ stream for 5 min, followed by dichloromethane (1 mL) and acetonitrile (1 mL). The suspension stirred at room temperature for 3 h, cooled to −30° C., to which point a solution of N-iodosuccinimide (61.4 mg, 0.273 mmol) in 0.5 mL acetonitrile (previously dried over 3 A activated molecular sieves), followed by trifluoromethanesulfonic acid (8.0 μL, 0.09 mmol). The final reaction mixture was stirred between −20 to −35° C. for 2 h, −15 to −20 for 2 h, and 0° C. for 1 h. The reaction was quenched by addition of triethylamine (0.1 mL) and sat. NaHCO₃ (5 mL) and sat. Na₂S₂O₃ (5 mL). The resultant mixture was extracted with ethyl acetate (4×5 mL ea), and the combined organic layers were dried over Na₂SO₄, filtered and concentrated to dry. The residue was semi-purified over a Biotage SNAP column (10 g) eluting with a gradient of 50 to 100%, ethyl acetate in heptane (10 CV) to provide a mixture of products upon concentration of the desired fraction to dryness. The mixture was finally purified by preparative TLC (silica gel, 4×0.5 mm plates) eluting with ethyl acetate. Then reverse phase HPLC to provide 328 (9.5 mg, 0.0063 mmol, 9.3%) (MW_{Calc}+H=1499.57; MW_{Obs}=1499.23) and 329 (9.0 mg, 0.0061 mmol, 9.0%) (MW_{Calc}+H=1499.57; MW_{Obs}=1499.50) after separately collecting the desired fractions and concentration to dryness.

To a stirred solution of (1R,2R)-3-(4-acetoxy-3,5-dimethylbenzamido)-1-((2R,3R,4S,6R)-4-acetoxy-3-(2-(4-cyclohexyl-1H-1,2,3-triazol-1-yl)acetamido)-6-(((2R,3S,4S,5R,6S)-4,5-diacetoxy-6-(((2R,3R,4S,5R,6R)-4,5-diacetoxy-2-(acetoxymethyl)-6-(2-(((benzyloxy)carbonyl)amino)ethoxy)tetrahydro-2H-pyran-3-yl)oxy)-3-hydroxytetrahydro-2H-pyran-2-yl)methoxy)-6-(methoxycarbonyl)tetrahydro-2H-pyran-2-yl)propane-1,2-diyl diacetate (328, 9.5 mg, 6.336 μmol) in MeOH (1.63 mL) at room temperature was added 1 N sodium hydroxide (200 μl, 0.20 mmol). The reaction mixture was stirred for 1 h, after which time it was neutralized with 1 N aqueous HCl (140 μl, 0.14 mmol) to ˜pH 7-8, and then diluted with methanol (2 mL). The solution was placed under N₂ atmosphere, followed by the addition of 20% palladium hydroxide on carbon (15 mg), degassed under a H₂ atmosphere (3×), and finally stirred under a H₂ atmosphere for 1 h. The final completed reaction was filtered through Celite (3 g), eluting with methanol (3×3 mL ea). The filtrate was concentrated and then purified over a reverse phase HPLC column eluting with acetonitrile in water to provide (2R,4S,5R,6R)-2-(((2R,3R,4S,5R,6S)-6-(((2R,3S,4R,5R,6R)-6-(2-aminoethoxy)-4,5-dihydroxy-2-(hydroxymethyl)tetrahydro-2H-pyran-3-yl)oxy)-3,4,5-trihydroxytetrahydro-2H-pyran-2-yl)methoxy)-5-(2-(4-cyclohexyl-1H-1,2,3-triazol-1-yl)acetamido)-6-((1R,2R)-1,2-dihydroxy-3-(4-hydroxy-3,5-dimethylbenzamido)propyl)-4-hydroxytetrahydro-2H-pyran-2-carboxylic acid (4.8 mg, 0.005 mmol, 78%) after combining the desired fractions and concentration to dryness. (MW_{Calc}+H=973.42; MW_{Obs}=973.20).

To a stirred solution of (2R,4S,5R,6R)-2-(((2R,3R,4S,5R,6S)-6-(((2R,3S,4R,5R,6R)-6-(2-aminoethoxy)-4,5-dihydroxy-2-(hydroxymethyl)tetrahydro-2H-pyran-3-yl)oxy)-3,4,5-trihydroxytetrahydro-2H-pyran-2-yl)methoxy)-5-(2-(4-cyclohexyl-1H-1,2,3-triazol-1-yl)acetamido)-6-((1R,2R)-1,2-dihydroxy-3-(4-hydroxy-3,5-dimethylbenzamido)propyl)-4-hydroxytetrahydro-2H-pyran-2-carboxylic acid (1.00 mg, 1.03 μmol) in water (0.5 ml, 27.754 mmol) was added 0.1 M aqueous sodium bicarbonate (0.023 ml, 2.30 μmol) followed by 2,5-dioxopyrrolidin-1-yl 17-oxo-21-((3aS,4S,6aR)-2-oxohexahydro-1H-thieno[3,4-d]imidazol-4-yl)-4,7,10,13-tetraoxa-16-azahenicosan-1-oate (0.666 mg, 1.131 μmol) in DMF (0.067 mL). The reaction mixture was stirred at 0° C. for 1 h, followed by an additional solution 2,5-dioxopyrrolidin-1-yl 17-oxo-21-((3aS,4S,6aR)-2-oxohexahydro-1H-thieno[3,4-d]imidazol-4-yl)-4,7,10,13-tetraoxa-16-azahenicosan-1-oate (0.13 mg, 0.22 μmol) in DMF (0.01 mL) and warmed to room temperature. The final mixture was stirred for 1 h, after which time it was diluted with methanol (1 mL), concentrated and purified by reverse phase HPLC to provide A-375 (1.20 mg, 0.83 μmol, 81%) after combining the desired fraction and concentration to dryness. (MW_{Calc}+H=1446.64; MW_{Obs}=1446.22)

### Biological Assays

### CD33 Competitive Binding Assay for HTS Drug Screen

This is a competitive binding assay that uses recombinant CD33 immobilized to a plate and a fluorescently-labeled multivalent sialic acid analog as a probe. Binding of drug to the site on CD33 to which sialic acid binds is detected by reduced binding of the fluorescent probe.

Recombinant human CD33 protein (for example Acro Biosystems cat #CD3-H5257) was added at a concentration of 10 μg/ml in 20 mM acetate buffer (pH5.5) to a black plastic plate (Corning 4510) and allowed to incubate overnight at 4° C. The next day, the wells were washed 3 times with PBS containing 0.02% Tween-20. This was followed by a 30-minute incubation with PBS containing 0.2% Tween-20 and 0.5% BSA to block non-specific binding to the well. This buffer is removed and replaced with 10 μl of the same buffer, which is also used as the assay buffer.

The fluorescent multivalent sialic acid probe was prepared as a 4× concentration as follows. A biotinylated sialic acid analog A-375 was mixed with biotin-4-Fluorescein isothiocyanate (biotin-4-FITC) at a 3:1 molar ratio with final concentrations (4× stock) of 60 μM A-375 and 20 μM biotin-4-FITC. After these were mixed, 20 μM of neutravidin was added, which should form complexes consisting of neutravidin, fluorescein and sialic acid with an average complex consisting of 1 neutravidin, 1 FITC and 3 sialic acid analogs. The multimerization of the sialic acid analog is critical to the assay and improves the affinity of the sialic acid analog several orders of magnitude.

A test compound, for example A-001, is added at 5 μl to the 10 μl assay buffer already in the well. After a brief incubation, 5 μl of the 4× stock solution of sialic acid complexes was added. The final concentration of the complexes was therefore 5 μM neutravidin, 15 μM A-375 and 5 μM FITC. The plate was incubated at room temperature for 30 mins and then washed with PBS containing 0.2% Tween-20. Binding of the sialic acid complex is very stable, with an off-rate of several days. Bound complex was then detected using a standard plate reader to quantify FITC fluorescence.

### Differential Scanning Fluorimetry

Differential Scanning Fluorimetry (also known as DSF, Protein Thermal Shift Assay, Thermofluor Assay) was used to measure and compare the thermal melt or thermal denaturation temperature of recombinant human CD33 (Siglec-3, sialic acid binding Ig-like lectin 3) protein in the presence of 50 μM of A-001. In the absence of any compound, the CD33 protein has a thermal melt at approximately 55° C. under the method conditions described in this report. A significant increase in thermal melt (dTm D) of 12.2±0.7° C. was observed in the denaturation temperature of the CD33 protein in the presence of 50 μM of A-001. The increase in the thermal melt indicates that A-001 binds to and stabilizes the CD33 protein.

In the DSF assay, as the temperature increases, the protein gets gradually unfolded, exposing hydrophobic regions in which a fluorescent dye binds causing the fluorescence signal to increase. The midpoint of the unfolding transitions is called the thermal melt (Tm) or denaturation temperature and the Tm was defined as the peak apex in the derivative curve. Here, the Tm temperature of CD33 in the presence and absence of A-001 at a concentration of 50 μM was examined.

Recombinant Human Siglec-3/CD33 protein was purchased from Acros (catalog no. CD3-H5226) and reagents used in this study are shown in the Table 1 below.

| Solvent | Source | Catalog No. |
|---|---|---|
| Cell Culture Grade Water | Corning | 25-055-CV |
| 5M NaCl | | |
| Phosphate Buffer Solution | Sigma-Aldrich | P3619-1GA |
| 1X PBS without Calcium or Magnesium | Corning | 21-040-CV |
| 5000X Sypro Orange Protein Gel Stain | Sigma | S5692-500UL |

Lyophilized CD33 protein was received in 1 mg aliquots from Acros. The protein was diluted with 2.5 mL of sterile water to a concentration of 400 μg/mL (14.5 μM), aliquoted into 138 μL aliquots and stored as at −20° C. On the day of analysis, the protein was thawed and 14.5 M solution was diluted to 0.67 M using buffer containing 133 mM NaCl, 133 mM phosphate buffered solution and 13.9× Sypro Orange dye in water in a quantity to cover the amount of samples tested on the day of the analysis.

200 μM stock solutions of A-001 containing 0.2% DMSO in water was supplied by compound management in a 384-well plate format.

Assays were performed on a QuantStudio 12K Flex Real-Time PCR System using a temperature gradient from 25° C. to 95° C. at a rate of 0.05° C./min employing a fluorescence detector with excitation and emission filters at 470 and 586 n, respectively. Sample matrix were prepared by adding 7.5 μL of the protein buffer stock solution followed by addition of 2.5 μL of the 200 μM compound solutions in 384-well PCR plates to give a final volume of 10 μL per well consisting of 0.5 μM CD33 protein, 100 mM phosphate buffered solution pH 7.4, 100 mM NaCl, 10× SYPRO orange dye and 50 μM compound concentration (final DMSO concentration ˜0.2%). Using the Applied Biosystems® Protein Thermal Shift software (algorithm version 1.3.), the denaturation temperature or thermal melt (Tm) of the unbound and ligand bound CD33 protein was automatically assigned and the difference in thermal melt (ΔTm D) was calculated using the derivative curve by the Protein Thermal Shift software.

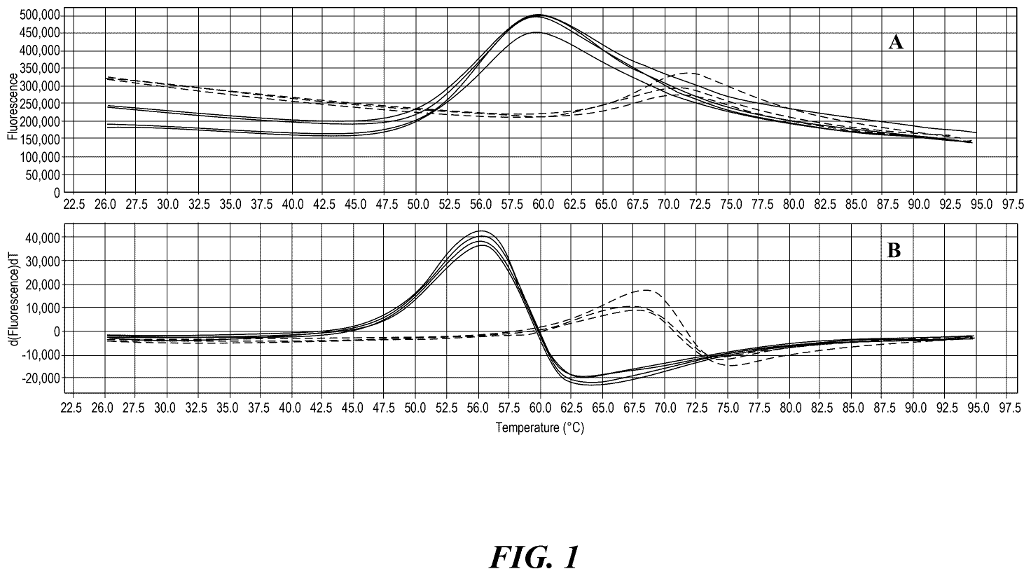

Recombinant human CD33 protein (0.5 μM) was incubated under buffered conditions in the presence of SYPRO orange dye, a temperature gradient was applied and a thermogram was produced by monitoring the fluorescence. The Tm was defined as the peak apex in the derivative curve in the study. As shown in FIG. 1, the Tm of CD33 protein alone (Gray lines) had an average value of approximate 55° C. Addition of 50 mM of A-001 (Blue lines) to the CD33 protein induced significant Tm increases (ΔTm D) of 12.2±0.7° C. The numerical results are summarized in the Table 2 below. The increase in dTm D indicates that A-001 binds to and stabilizes the His-CD33 protein.

TABLE 2
Summary of thermal shift results for
His-CD33 in the presence of A-001

| Ligand | ΔTm D (° C.) | Median ΔTm D (° C.) |
|---|---|---|
| A-001 | 12.2, 11.9, 12.9 | 12.2 ± 0.7 |

TABLE 3
Data for Compounds

| | | MS | | | DSF | EC₅₀* |
|---|---|---|---|---|---|---|
| # | Structure | ion | MW_{calc} | MW_{obs} | (dTmD) | (μM) |
| A-001 | 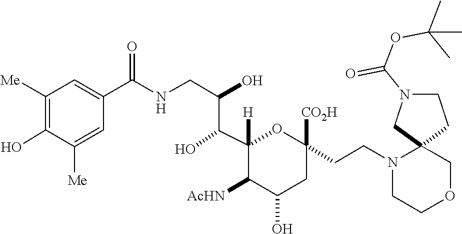 | H | 709.81 | 709.34 | 12.9 | 0.015 |
| A-002 | 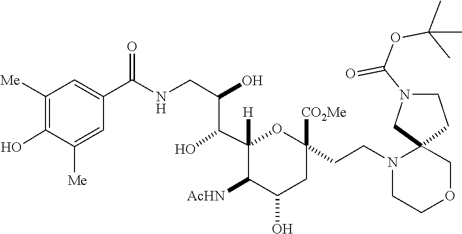 | H | 723.37 | 723.54 | −0.3 | 49.0 |
| A-003 | 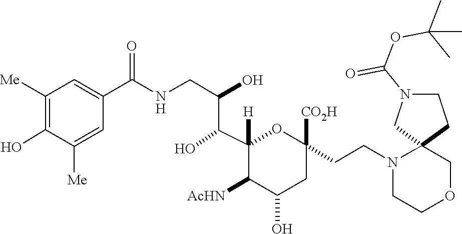 | H | 709.36 | 709.49 | 5.12 | 1.14 |
| A-004 | 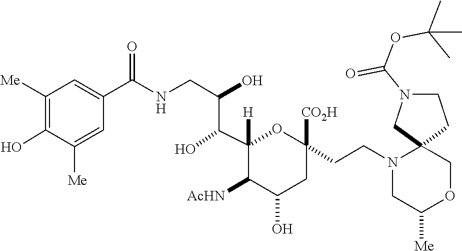 | H | 723.37 | 723.24 | 9.1 | 0.098 |
| A-005 | 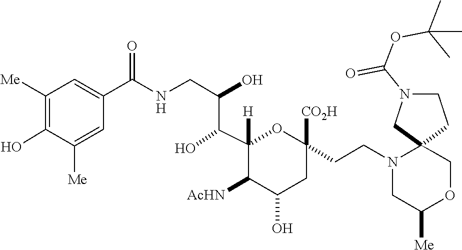 | H | 723.37 | 723.25 | 14.1 | 0.007 |
| A-006 | 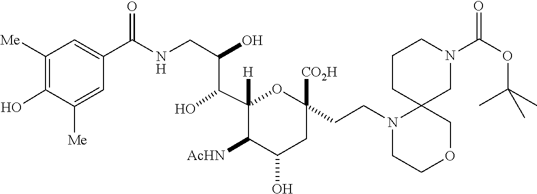 [SMILES: CC(=O)N[C@H]1[C@H]([C@H](O)[C@H](O)CNC(=O)c2cc(C)c(O)c(C)c2)O[C@@](CCN2CCOCC23CCCN(C(=O)OC(C)(C)C)C3)(C(=O)O)C[C@@H]1O] | H | 723.38 | 723.6 | | |
| A-007 | 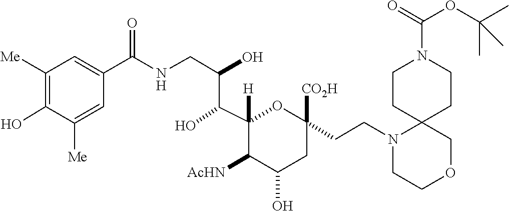 [SMILES: CC(=O)N[C@H]1[C@H]([C@H](O)[C@H](O)CNC(=O)c2cc(C)c(O)c(C)c2)O[C@@](CCN2CCOCC23CCN(C(=O)OC(C)(C)C)CC3)(C(=O)O)C[C@@H]1O] | H | 723.38 | 723.6 | | 0.811 |
| A-008 | 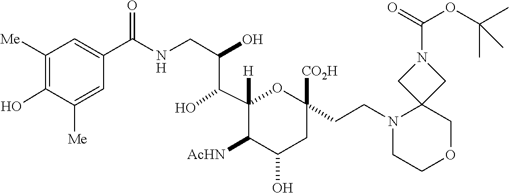 [SMILES: CC(=O)N[C@H]1[C@H]([C@H](O)[C@H](O)CNC(=O)c2cc(C)c(O)c(C)c2)O[C@@](CCN2CCOCC23CN(C(=O)OC(C)(C)C)C3)(C(=O)O)C[C@@H]1O] | H | 695.35 | 695.6 | | 0.538 |
| A-009 | 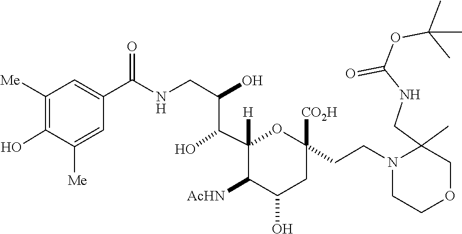 [SMILES: CC(=O)N[C@H]1[C@H]([C@H](O)[C@H](O)CNC(=O)c2cc(C)c(O)c(C)c2)O[C@@](CCN2CCOCC2(C)CNC(=O)OC(C)(C)C)(C(=O)O)C[C@@H]1O] | H | 697.4 | 697.7 | 6.1 | 2.00 |
| A-010 | 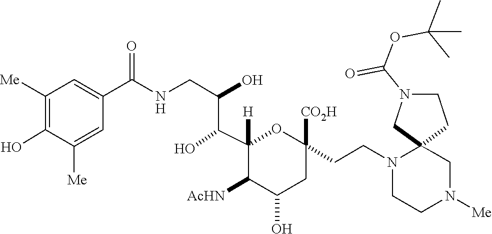 | H | 722.39 | 722.51 | 13.5 | 0.015 |
| A-011 | 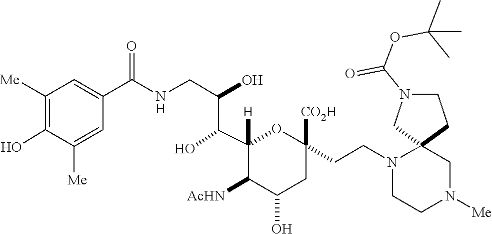 | H | 722.39 | 722.53 | 3.9 | 1.63 |
| A-012 | 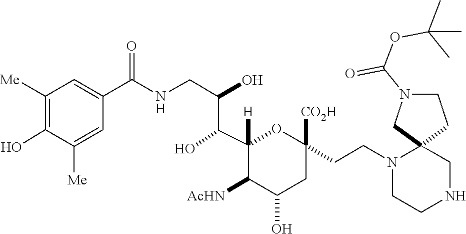 | H | 708.38 | 708.37 | 12.6 | 0.022 |
| A-013 | 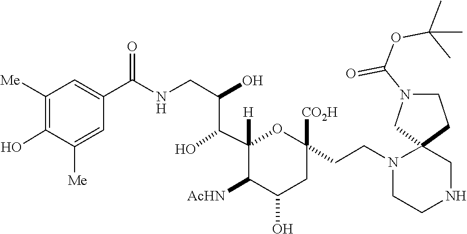 | H | 708.38 | 708.37 | 11.4 | 0.035 |
| A-014 | 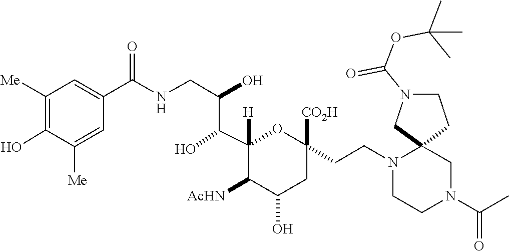 | H | 750.39 | 750.38 | 12.3 | 0.019 |
| A-015 | 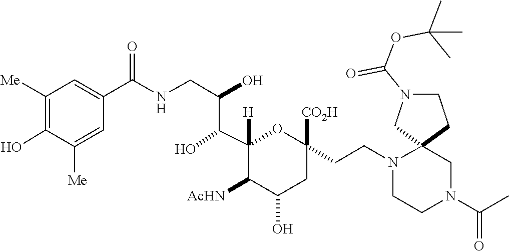 | H | 750.39 | 750.38 | 3.8 | 3.34 |
| A-016 | 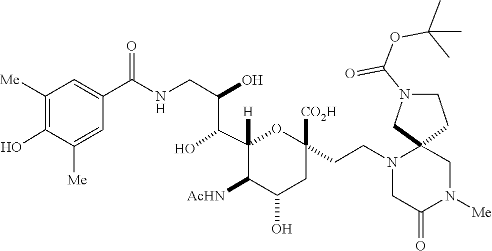 | H | 736.37 | 736.37 | 9.7 | 0.034 |
| A-017 | 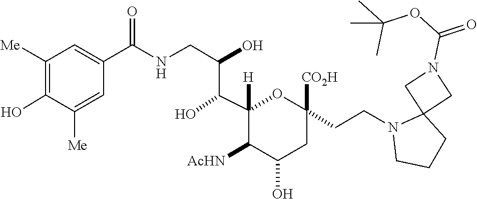 [SMILES: CC(=O)N[C@H]1[C@H]([C@H](O)[C@H](O)CNC(=O)c2cc(C)c(O)c(C)c2)O[C@@](CCN2CCCC23CN(C(=O)OC(C)(C)C)C3)(C(=O)O)C[C@@H]1O] | H | 679.35 | 679.35 | 1.2 | 1.56 |
| A-018 | 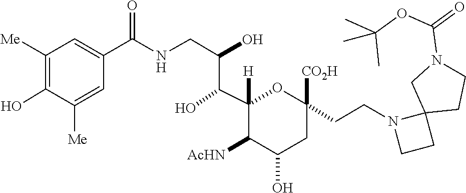 [SMILES: CC(=O)N[C@H]1[C@H]([C@H](O)[C@H](O)CNC(=O)c2cc(C)c(O)c(C)c2)O[C@@](CCN2CCC23CCN(C(=O)OC(C)(C)C)C3)(C(=O)O)C[C@@H]1O] | H | 679.35 | 679.39 | | 0.162 |
| A-019 | 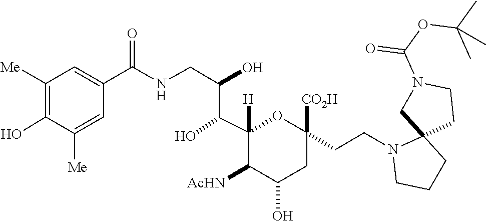 | H | 693.37 | 693.40 | | 0.028 |
| A-020 | 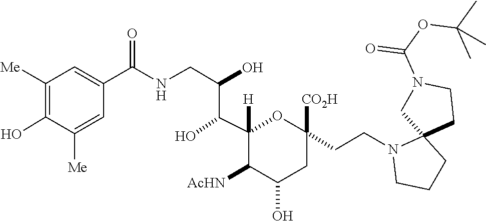 | H | 693.37 | 693.39 | | 0.472 |
| A-021 | 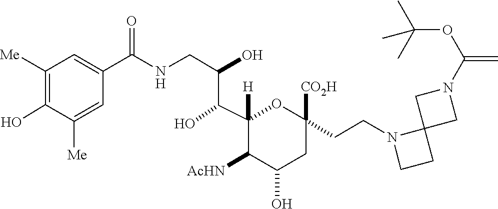 [SMILES: C=C(OC(C)(C)C)N1CC2(CCN2CC[C@]2(C(=O)O)C[C@H](O)[C@@H](NC(C)=O)[C@H]([C@H](O)[C@H](O)CNC(=O)c3cc(C)c(O)c(C)c3)O2)C1] | H | 665.34 | 665.33 | | 0.887 |
| A-022 | 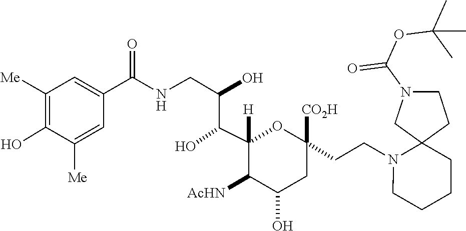 [SMILES: CC(=O)N[C@H]1[C@H]([C@H](O)[C@H](O)CNC(=O)c2cc(C)c(O)c(C)c2)O[C@@](CCN2CCCCC23CCN(C(=O)OC(C)(C)C)C3)(C(=O)O)C[C@@H]1O] | H | 707.38 | | | 2.93 |
| A-023 | 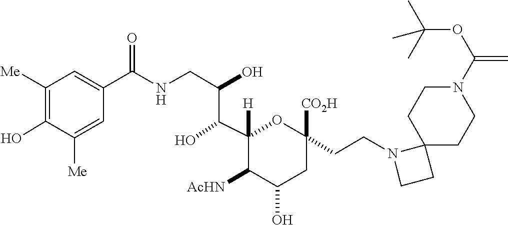 [SMILES: C=C(OC(C)(C)C)N1CCC2(CC1)CCN2CC[C@]1(C(=O)O)C[C@H](O)[C@@H](NC(C)=O)[C@H]([C@H](O)[C@H](O)CNC(=O)c2cc(C)c(O)c(C)c2)O1] | H | 693.37 | 693.36 | | 0.176 |
| A-024 | 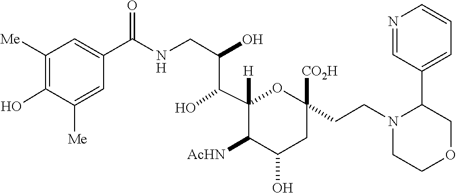 [SMILES: CC(=O)N[C@H]1[C@H]([C@H](O)[C@H](O)CNC(=O)c2cc(C)c(O)c(C)c2)O[C@@](CCN2CCOCC2c2cccnc2)(C(=O)O)C[C@@H]1O] | H | 631.29 | 631.37 | 1.1 | 7.94 |
| A-025 | 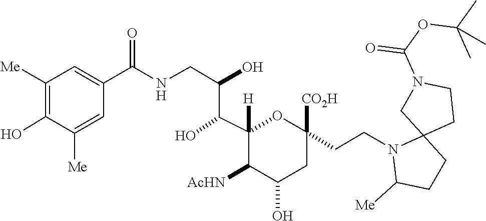 [SMILES: CC(=O)N[C@H]1[C@H]([C@H](O)[C@H](O)CNC(=O)c2cc(C)c(O)c(C)c2)O[C@@](CCN2C(C)CCC23CCN(C(=O)OC(C)(C)C)C3)(C(=O)O)C[C@@H]1O] | H | 707.38 | 707.38 | 10.0 | 0.115 |
| A-026 | 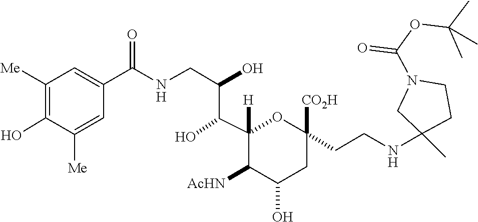 [SMILES: CC(=O)N[C@H]1[C@H]([C@H](O)[C@H](O)CNC(=O)c2cc(C)c(O)c(C)c2)O[C@@](CCNC2(C)CCN(C(=O)OC(C)(C)C)C2)(C(=O)O)C[C@@H]1O] | H | 667.35 | | 9.2 | 0.83 |
| A-027 | 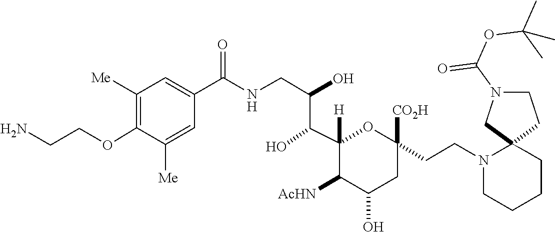 | H | 752.88 | 752.66 | 10.4 | 0.064 |
| A-028 | 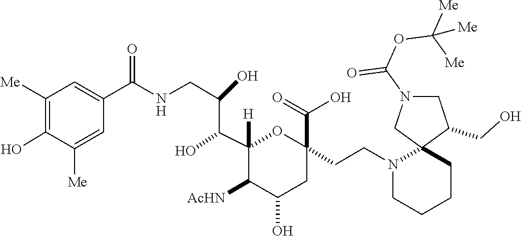 | H | 737.39 | 737.33 | 8.1 | 0.159 |
| A-029 | 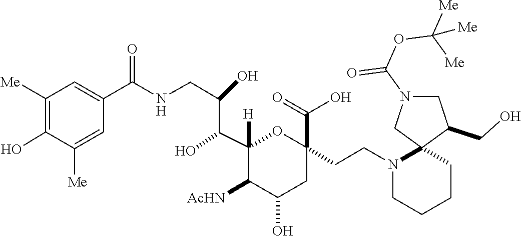 | H | 737.39 | 737.31 | 2.1 | 9.73 |
| A-030 | 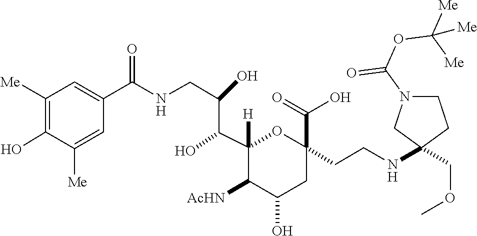 | H | 697.36 | 697.19 | | 0.049 |
| A-031 | 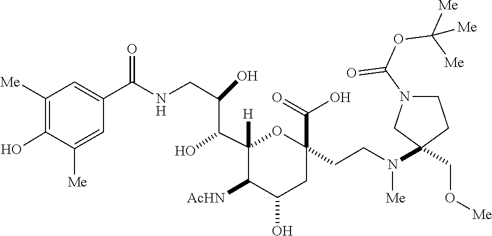 | H | 711.38 | 711.20 | | 0.070 |
| A-032 | 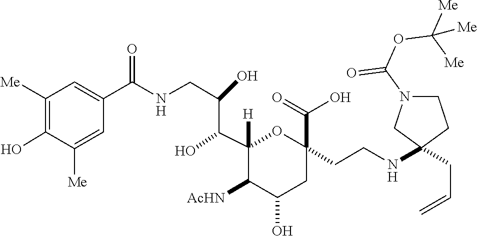 | H | 693.37 | 693.36 | 8.4 | 0.181 |
| A-033 | 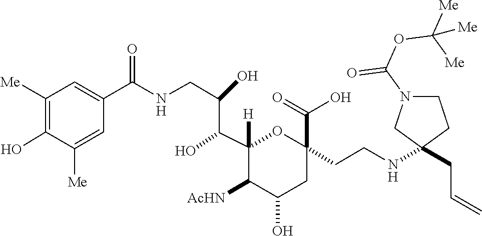 | H | 693.37 | 693.14 | 6.1 | 1.61 |
| A-034 | 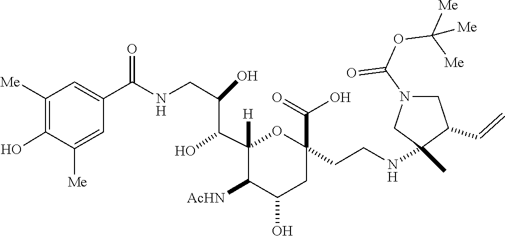 | H | 693.37 | 693.34 | 6.7 | 0.880 |
| A-035 | 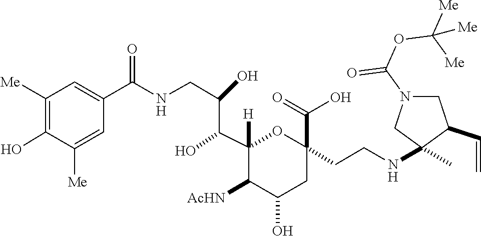 | H | 693.37 | 693.35 | 7.4 | 0.413 |
| A-036 | 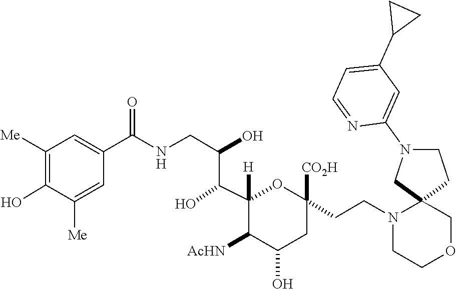 | H | 726.37 | 726.36 | 12.5 | 0.016 |
| A-037 | 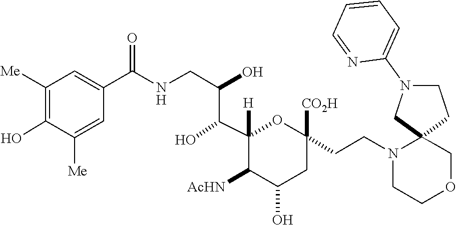 | H | 686.34 | 686.33 | 9.6 | 0.039 |
| A-038 | 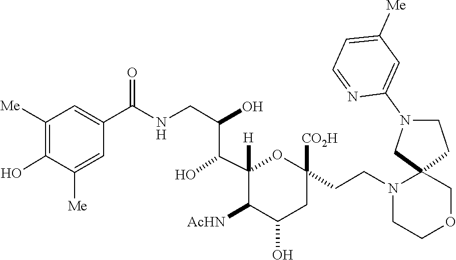 | H | 700.35 | 700.35 | | 0.053 |
| A-039 | 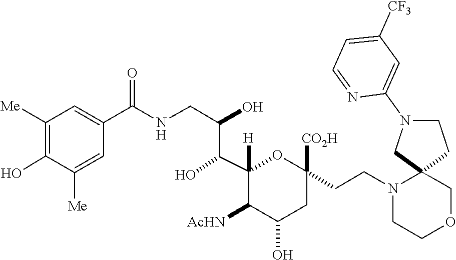 | H | 754.32 | 753.32 | | 0.095 |
| A-040 | 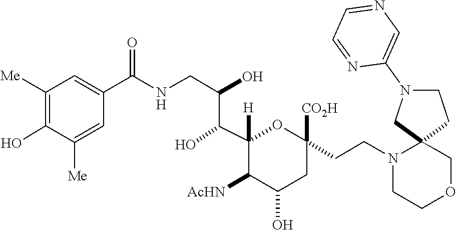 | H | 687.33 | 687.33 | 6.3 | 0.363 |
| A-041 | 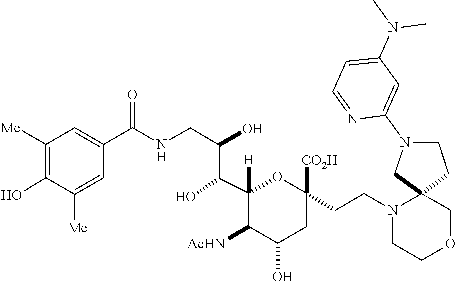 | H | 729.38 | 729.37 | | 0.580 |
| A-042 | 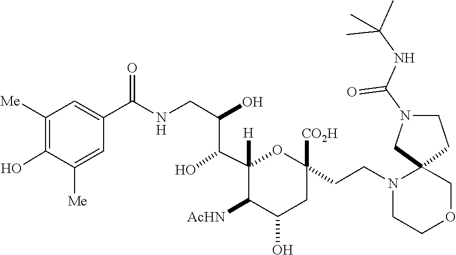 | H | 708.38 | 708.37 | 13.3 | 0.012 |
| A-043 | 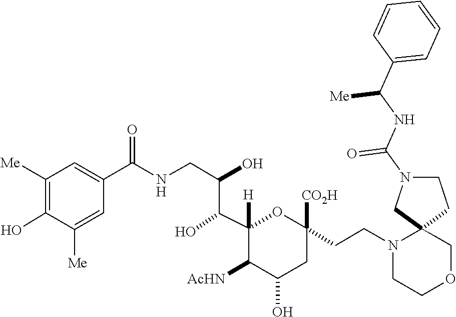 | H | 756.38 | 756.52 | 13.2 | 0.013 |
| A-044 | 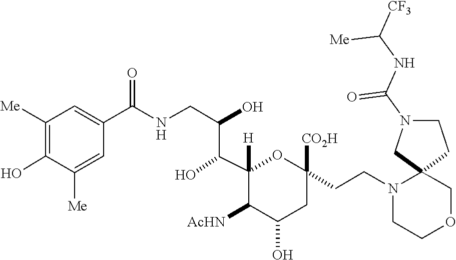 | H | 748.33 | 748.33 | 11.5 | 0.019 |
| A-045 | 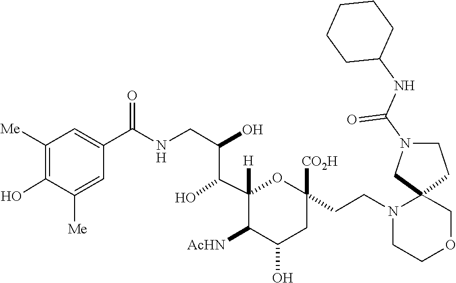 | H | 734.39 | 734.39 | 12.6 | 0.019 |
| A-046 | 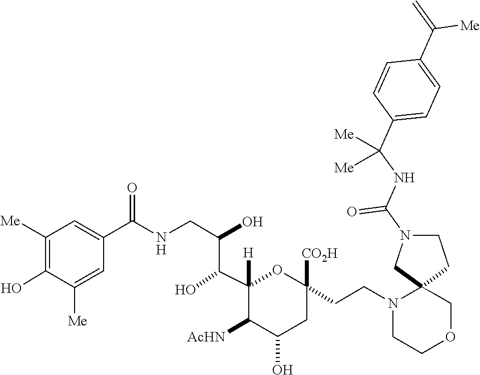 | H | 810.42 | 810.42 | | 0.063 |
| A-047 | 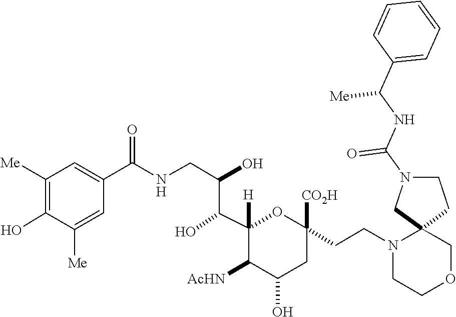 | H | 756.38 | 756.37 | 12.9 | 0.022 |
| A-048 | 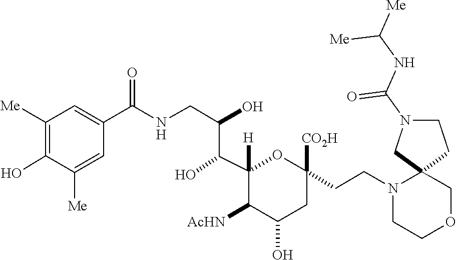 | H | 694.36 | 694.36 | 12.5 | 0.024 |
| A-049 | 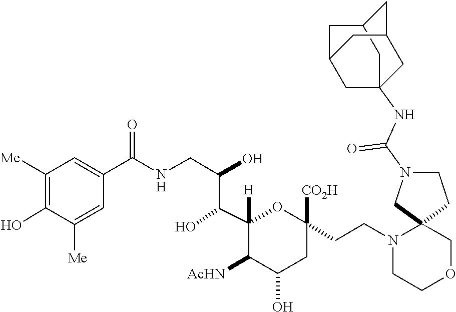 | H | 786.42 | 786.42 | 13.5 | 0.041 |
| A-050 | 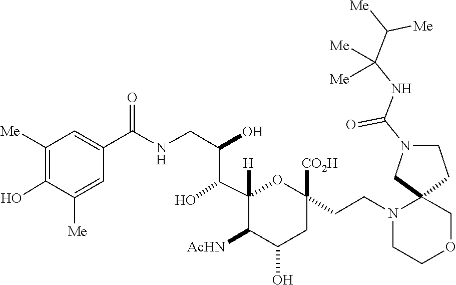 | H | 736.41 | 736.41 | | 0.020 |
| A-051 | 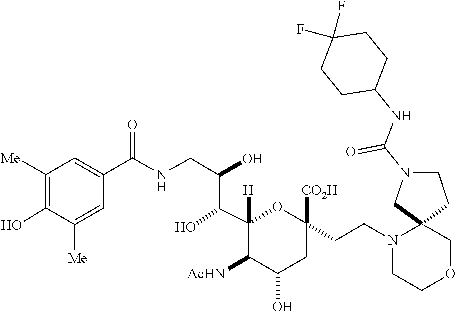 | H | 770.37 | 770.37 | 10.8 | 0.064 |
| A-052 | 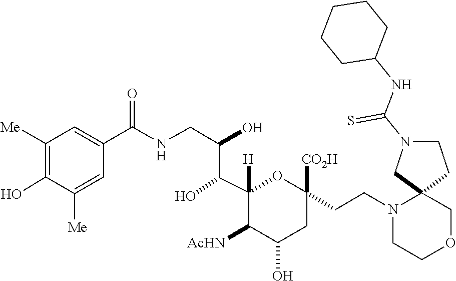 | H | 750.37 | 750.37 | 9.9 | 0.066 |
| A-053 | 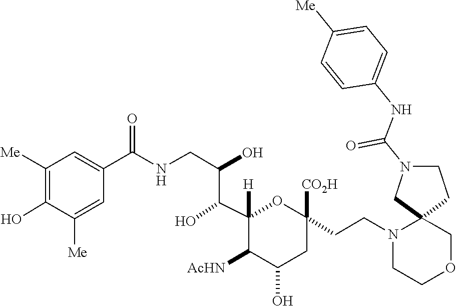 | H | 742.36 | 742.36 | 9.2 | 0.147 |
| A-054 | 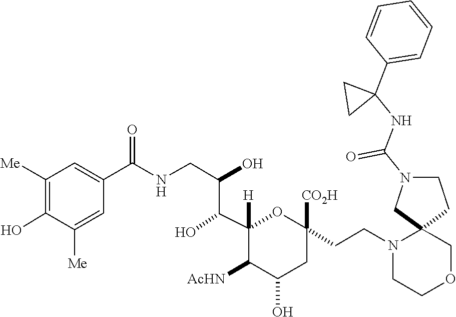 | H | 768.38 | 768.37 | | 0.147 |
| A-055 | 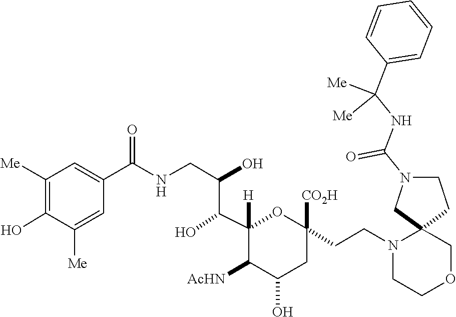 | H | 770.39 | 770.39 | | 0.247 |
| A-056 | 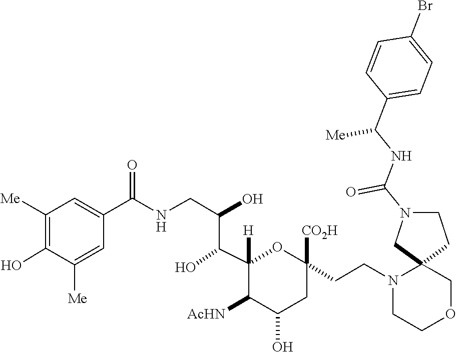 | H | 836.29 | 836.3 | | 0.153 |
| A-057 | 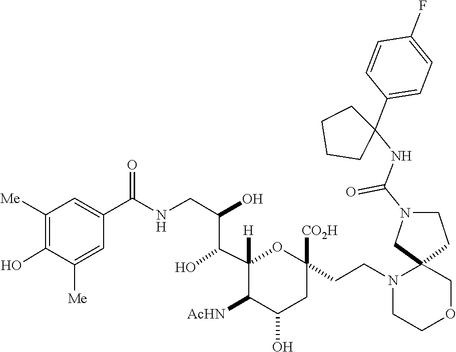 | H | 814.40 | 814.5 | 9.7 | 0.265 |
| A-058 | 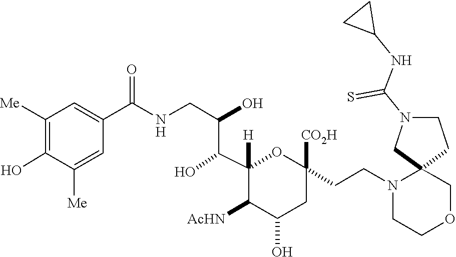 | H | 708.32 | 708.5 | 5.0 | 1.38 |
| A-059 | 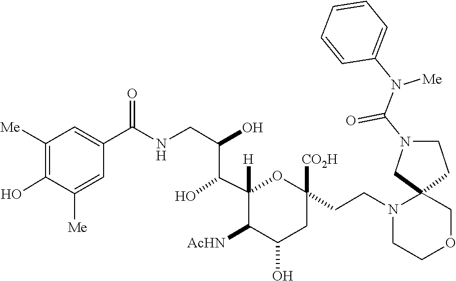 | H | 742.36 | 742.6 | 9.3 | 0.060 |
| A-060 | 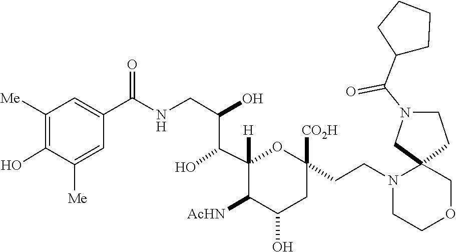 | H | 705.37 | 705.6 | 11.2 | 0.065 |
| A-061 | 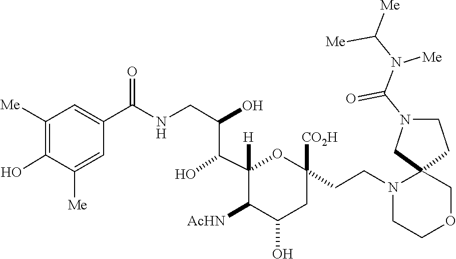 | H | 708.38 | 708.37 | 7.5 | 0.235 |
| A-062 | 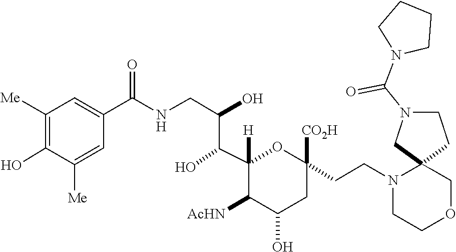 | H | 706.36 | 706.6 | 7.2 | 0.833 |
| A-063 | 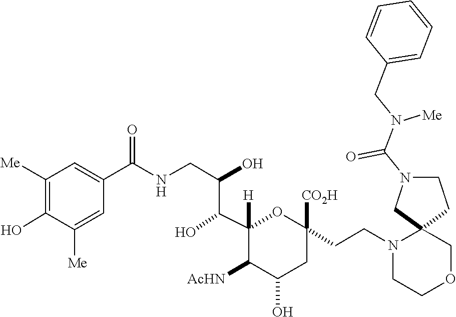 | H | 756.38 | 756.6 | 6.3 | 0.323 |
| A-064 | 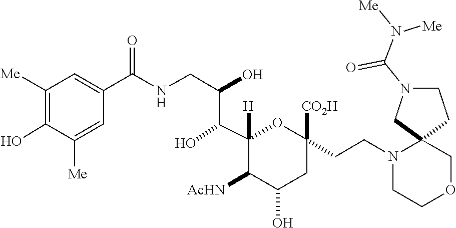 | H | 680.35 | 680.6 | 3.5 | 3.50 |
| A-065 | 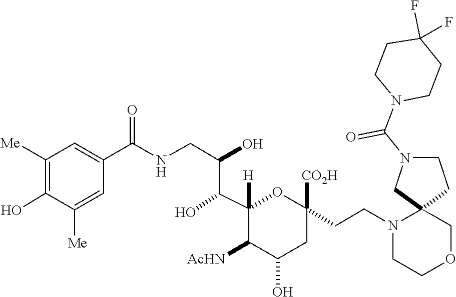 | H | 756.36 | 756.6 | 6.1 | 0.739 |
| A-066 | 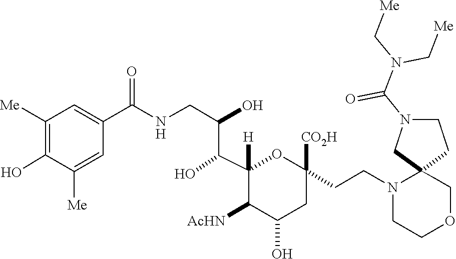 | H | 708.38 | 708.6 | 8.4 | 0.156 |
| A-067 | 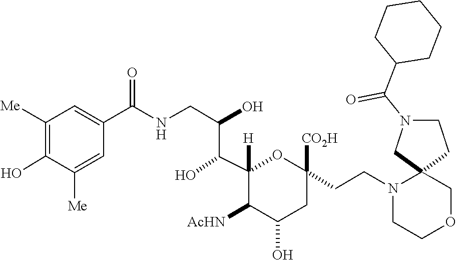 | H | 719.38 | 719.6 | | 0.063 |
| A-068 | 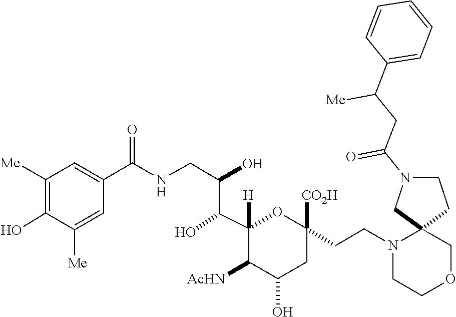 | H | 755.38 | | | 0.101 |
| A-069 | 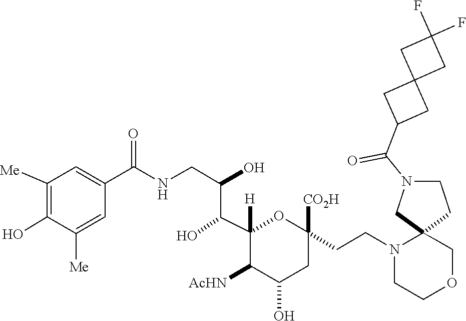 | H | 767.36 | 767.6 | 8.3 | 0.123 |
| A-070 | 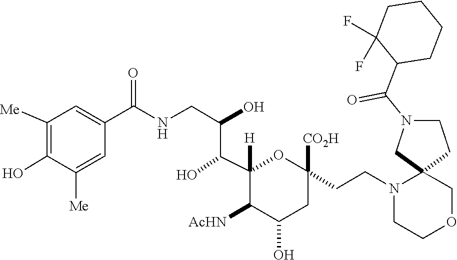 | H | 755.36 | 755.6 | 12.0 | 0.031 |
| A-071 | 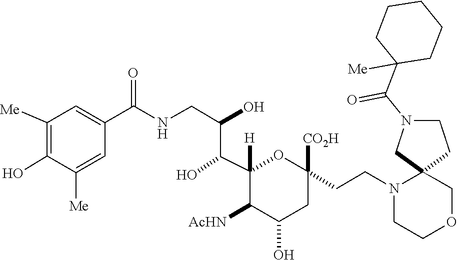 | H | 733.40 | 733.6 | 11.0 | 0.041 |
| A-072 | 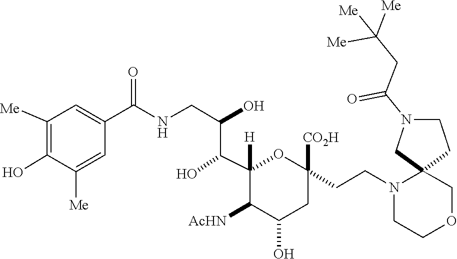 | H | 707.38 | 707.52 | | 0.230 |
| A-073 | 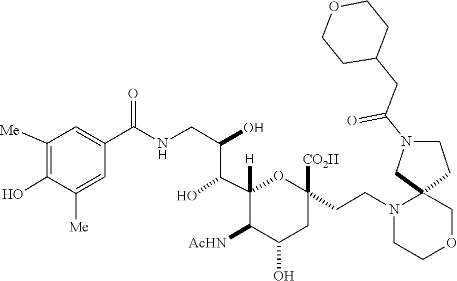 | H | 735.38 | 735.6 | | 0.206 |
| A-074 | 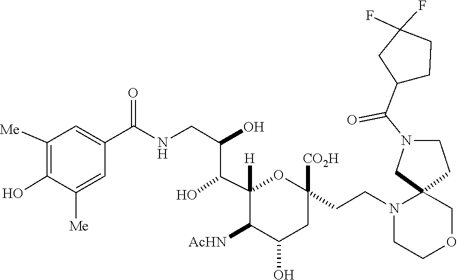 | H | 741.35 | 741.6 | | 0.263 |
| A-075 | 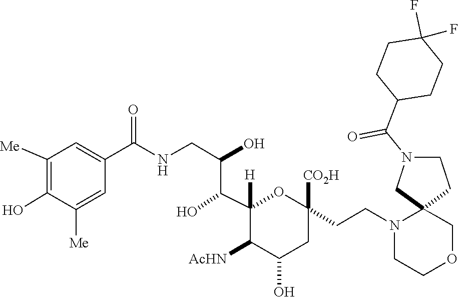 | H | 755.36 | 755.6 | | 0.099 |
| A-076 | 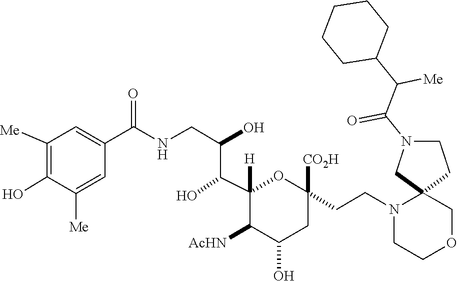 | H | 747.41 | | | 0.226 |
| A-077 | 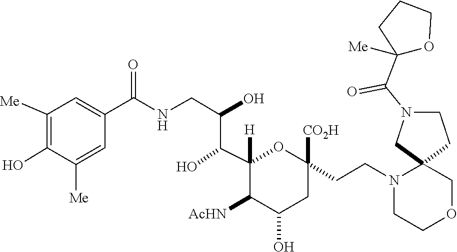 | H | 721.36 | 721.6 | | 0.283 |
| A-078 | 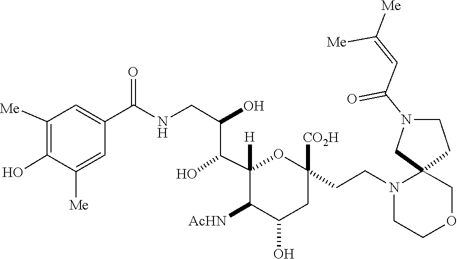 | H | 691.35 | 691.6 | 9.4 | 0.115 |
| A-079 | 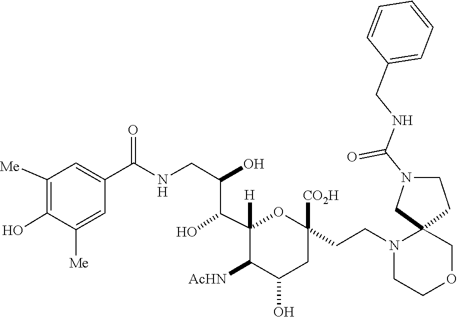 | H | 742.36 | | 9.1 | 0.152 |
| A-080 | 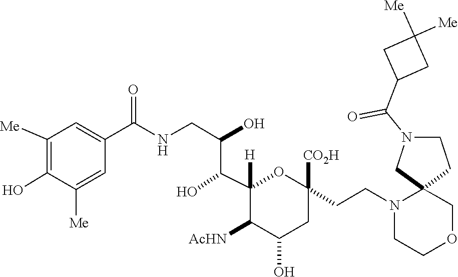 | H | 719.38 | 719.6 | 10.2 | 0.068 |
| A-081 | 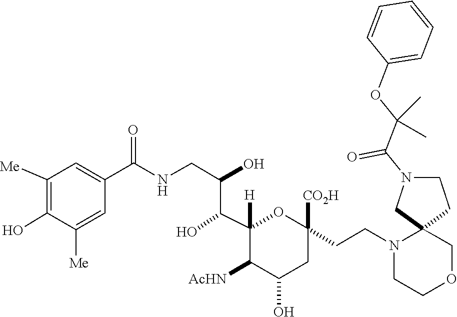 | H | 771.38 | 771.7 | | 1.95 |
| A-082 | 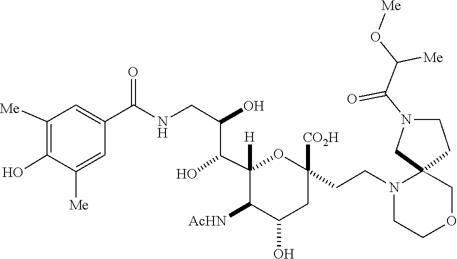 | H | 695.35 | 695.6 | | >5.0 |
| A-083 | 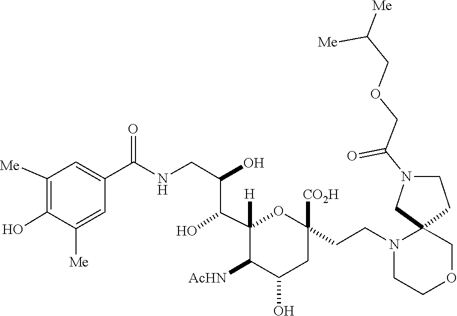 | H | 723.38 | 723.7 | | 1.00 |
| A-084 | 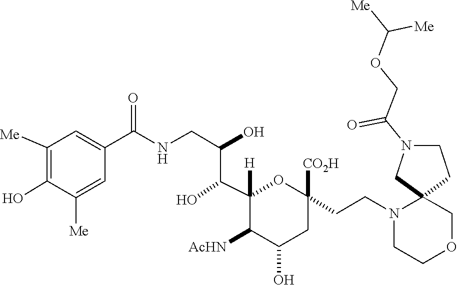 | H | 709.36 | 709.7 | | 1.56 |
| A-085 | 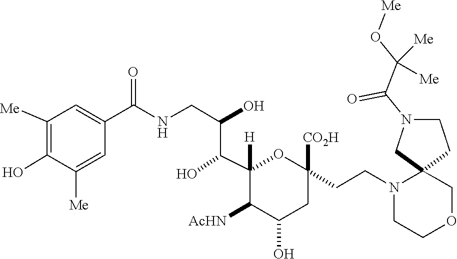 | H | 709.36 | | | 1.28 |
| A-086 | 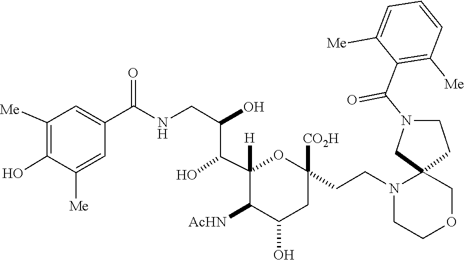 | H | 741.37 | 741.6 | | >5.0 |
| A-087 | 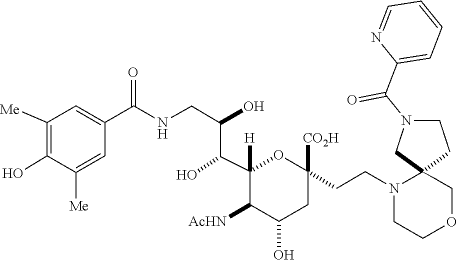 | H | 714.33 | 714.33 | | 5.26 |
| A-088 | 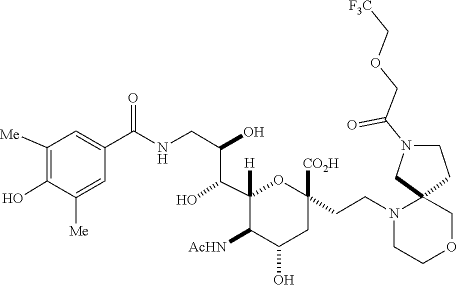 | H | 749.32 | 749.6 | | 1.95 |
| A-089 | 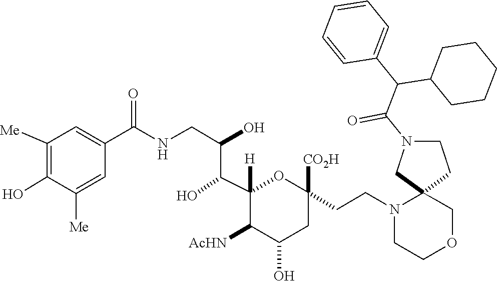 | H | 809.43 | 809.7 | | 0.291 |
| A-090 | 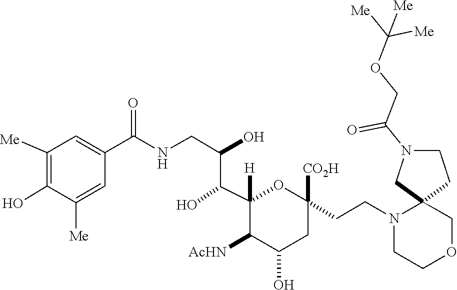 | H | 723.38 | 723.7 | | 3.12 |
| A-091 | 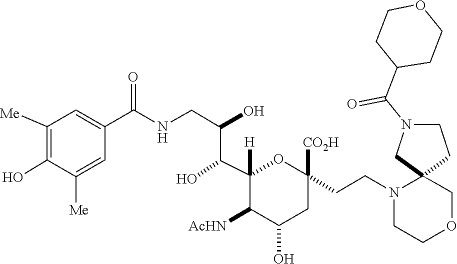 | H | 721.36 | 721.6 | | 0.452 |
| A-092 | 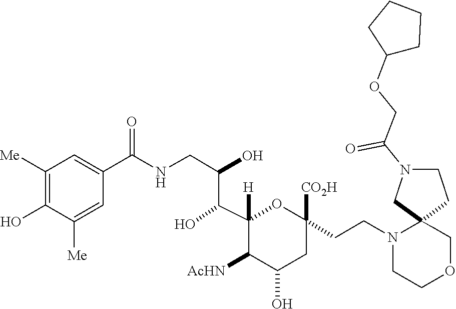 | H | 735.38 | 735.6 | | 2.05 |
| A-093 | 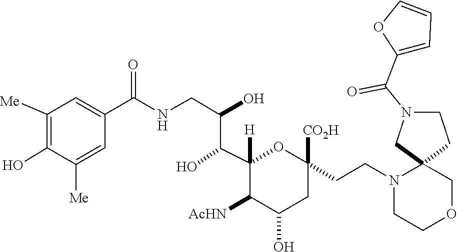 | H | 703.31 | 703.6 | | 0.892 |
| A-094 | 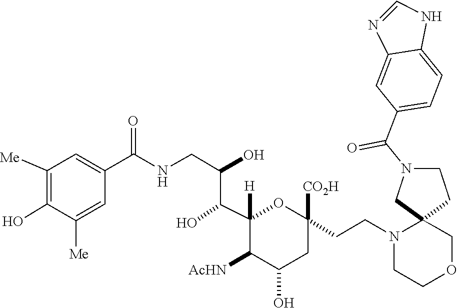 | H | 753.34 | 753.34 | | 2.33 |
| A-095 | 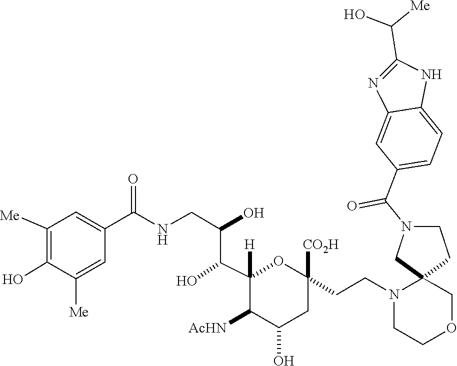 | H | 797.37 | 797.6 | | 1.69 |
| A-096 | 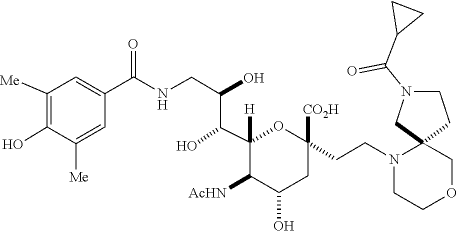 | H | 677.34 | 677.6 | 7.6 | 0.454 |
| A-097 | 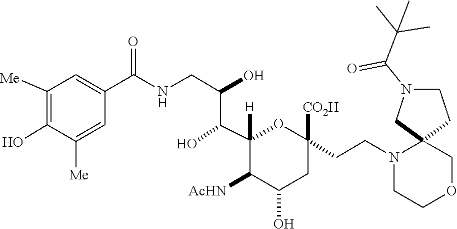 | H | 693.37 | 693.6 | 8.25 | 0.430 |
| A-098 | 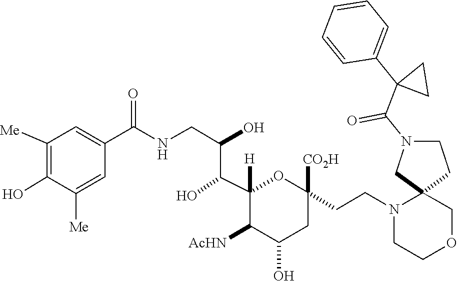 | H | 753.37 | 753.6 | 6.9 | 0.685 |
| A-099 | 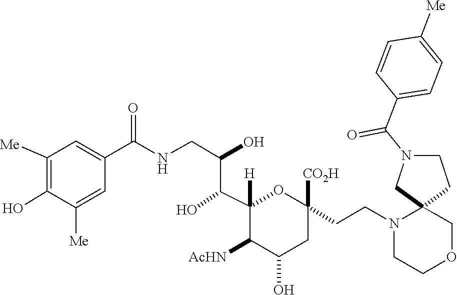 | H | 727.35 | 727.6 | 7.3 | 0.342 |
| A-100 | 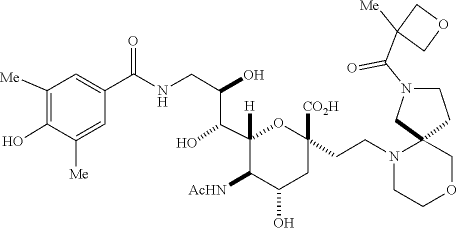 | H | 707.35 | 707.6 | 5.6 | 2.34 |
| A-101 | 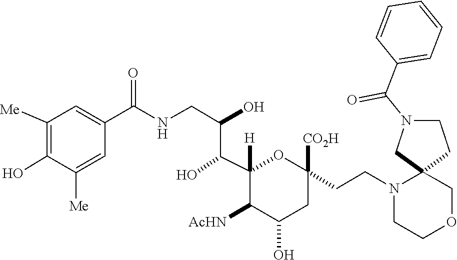 | H | 713.34 | 713.6 | 7.4 | 0.921 |
| A-102 | 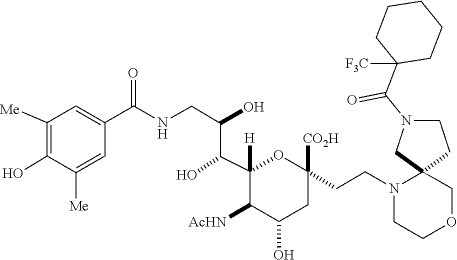 | H | 787.37 | 787.5 | 7.5 | 0.397 |
| A-103 | 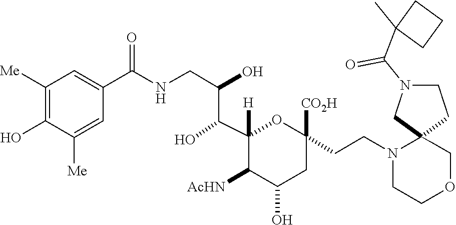 | H | 705.37 | 705.5 | 6.6 | 1.02 |
| A-104 | 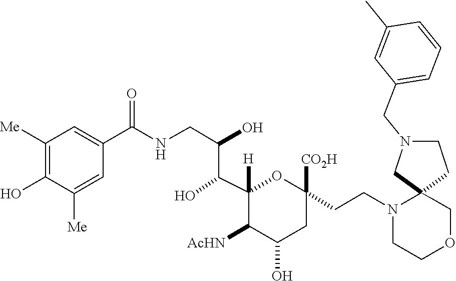 | H | 713.37 | 713.6 | | >10.0 |
| A-105 | 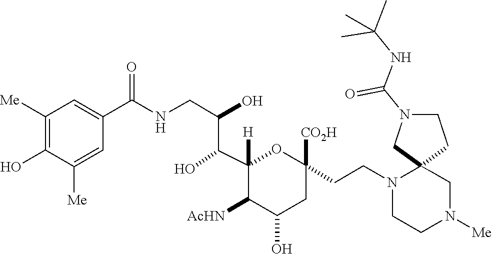 | H | 721.41 | 721.5 | | 0.008 |
| A-106 | 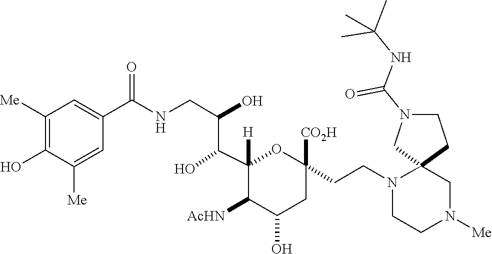 | H | 721.41 | 721.5 | | 1.95 |
| A-107 | 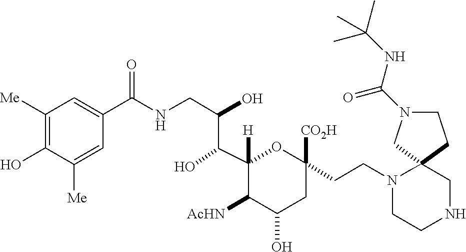 | H | 707.39 | 707.5 | | 0.379 |
| A-108 | 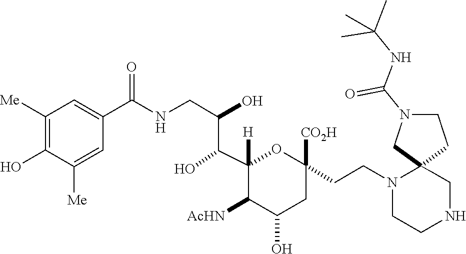 | H | 707.39 | 707.5 | | 0.026 |
| A-109 | 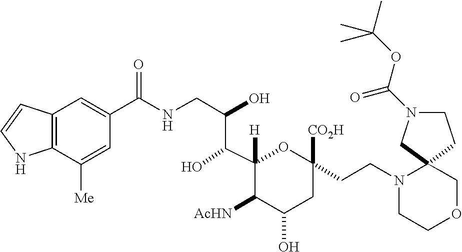 | H | 718.36 | 718.5 | 16.6 | 0.006 |
| A-110 | 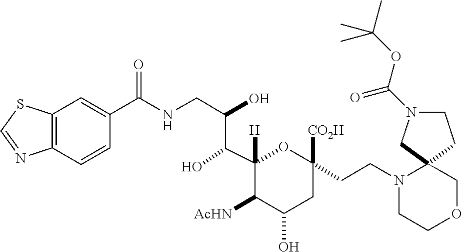 | H | 722.30 | 722.5 | 12.2 | 0.030 |
| A-111 | 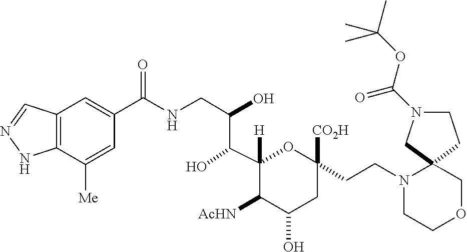 | H | 719.36 | 705.19 | 12.5 | 0.016 |
| A-112 | 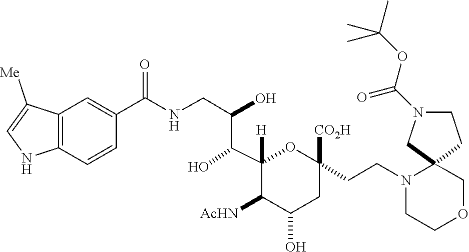 | H | 718.36 | 718.41 | 12.5 | 0.022 |
| A-113 | 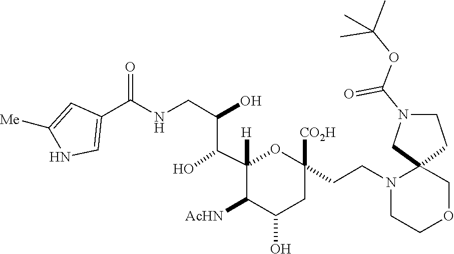 | H | 668.35 | 668.36 | 11.1 | 0.034 |
| A-114 | 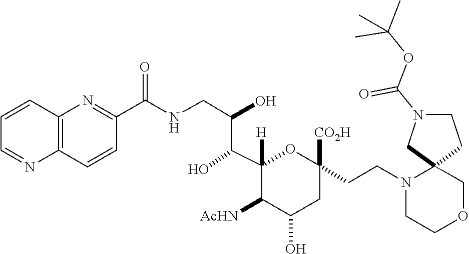 | H | 717.34 | 717.31 | 12.4 | 0.022 |
| A-115 | 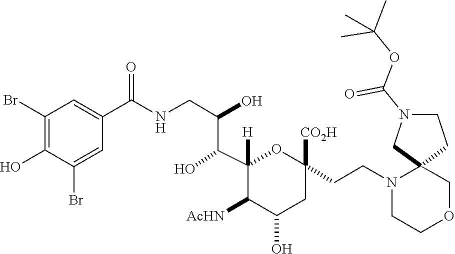 | H | 839.15 | 839.25 | 15.1 | 0.007 |
| A-116 | 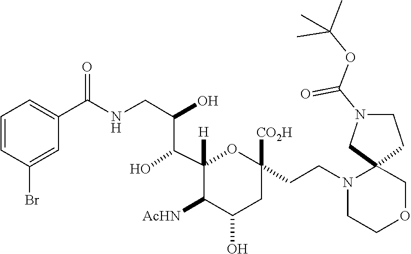 | H | 744.24 | 745.3 | 12.4 | 0.042 |
| A-117 | 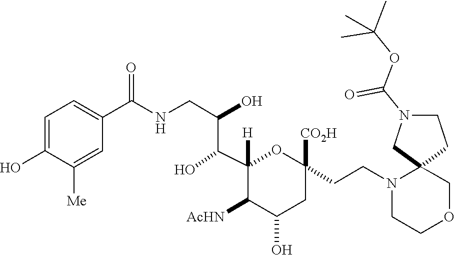 | H | 695.35 | 695.4 | 12.4 | 0.028 |
| A-118 | 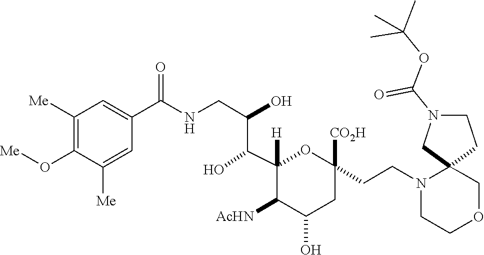 | H | 723.38 | 723.5 | 12.1 | 0.060 |
| A-119 | 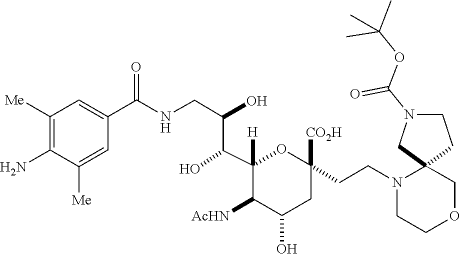 | H | 708.38 | 708.5 | 14.0 | 0.017 |
| A-120 | 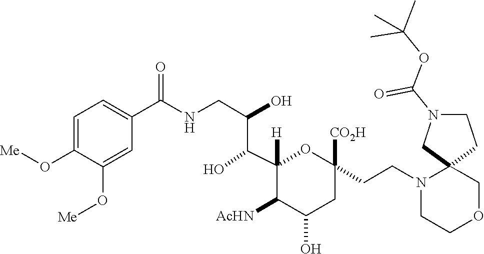 | H | 725.35 | 725.5 | 12.2 | 0.040 |
| A-121 | 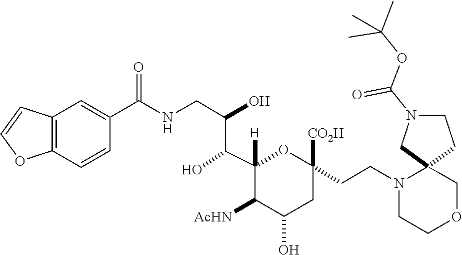 | H | 705.33 | 705.4 | 13.6 | 0.029 |
| A-122 | 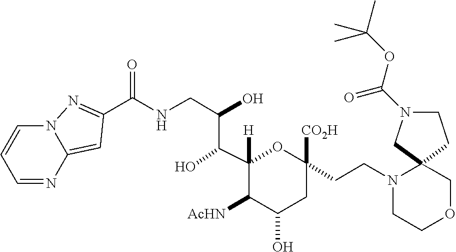 | H | 706.34 | 706.4 | 12.4 | 0.032 |
| A-123 | 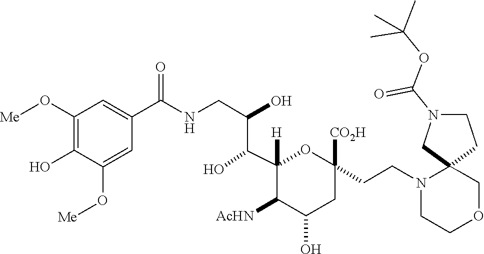 | H | 739.37 | 741.4 | 11.0 | 0.068 |
| A-124 | 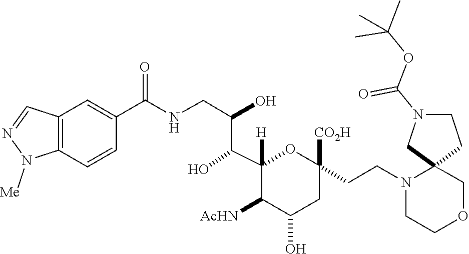 | H | 719.36 | 719.5 | 13.4 | 0.028 |
| A-125 | 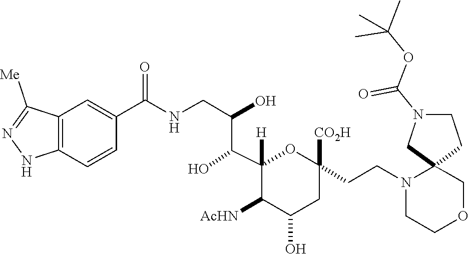 | H | 719.36 | 719.5 | 13.3 | 0.023 |
| A-126 | 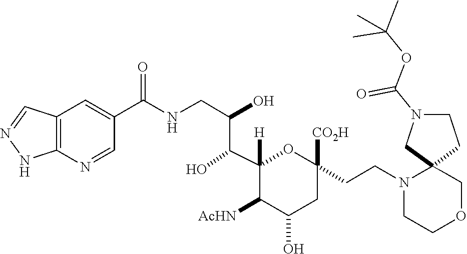 | H | 706.34 | 706.4 | 11.2 | 0.050 |
| A-127 | 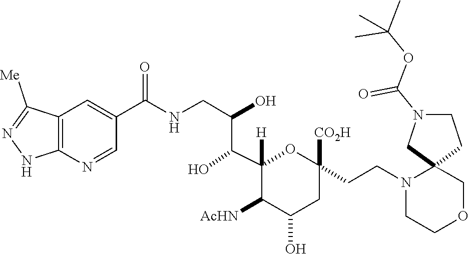 | H | 720.35 | 720.5 | 11.7 | 0.059 |
| A-128 | 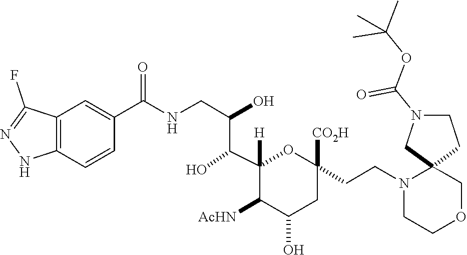 | H | 723.33 | 723.5 | 11.5 | 0.050 |
| A-129 | 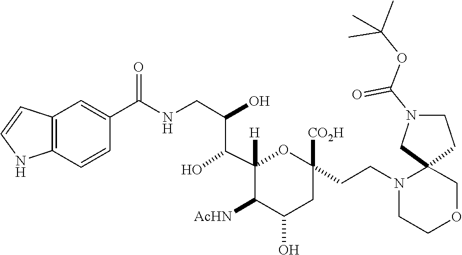 | H | 704.35 | 704.5 | 15.0 | 0.010 |
| A-130 | 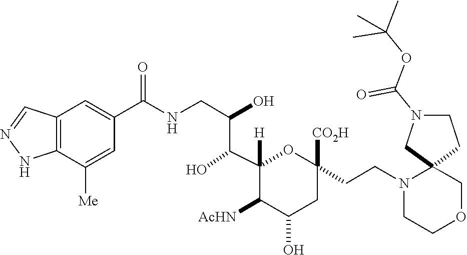 | H | 719.36 | 719.4 | | 0.009 |
| A-131 | 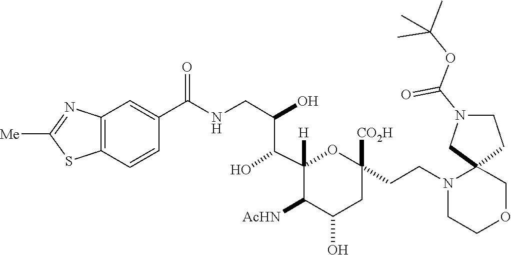 | H | 736.32 | 736.4 | | 0.024 |
| A-132 | 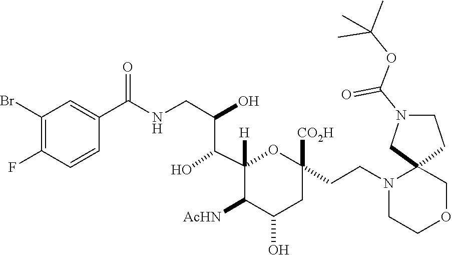 | H | 763.23 | 763.3 | 12.4 | 0.051 |
| A-133 | 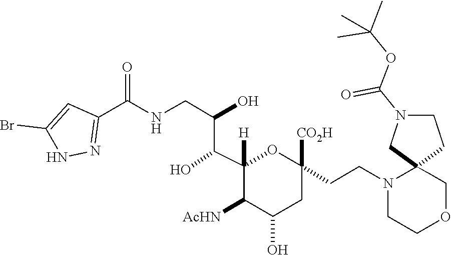 | H | 735.23 | 735.3 | 11.4 | 0.046 |
| A-134 | 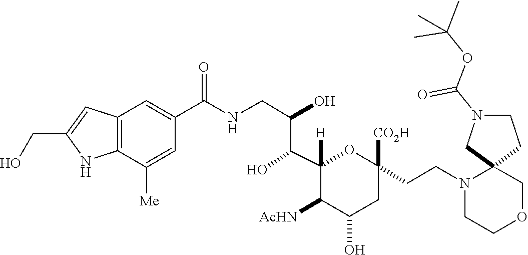 | H | 748.38 | 748.5 | 15.8 | 0.014 |
| A-135 | 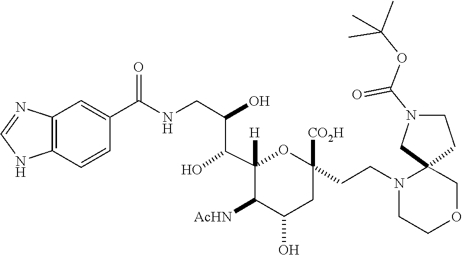 | H | 705.35 | 705.5 | 9.6 | 0.101 |
| A-136 | 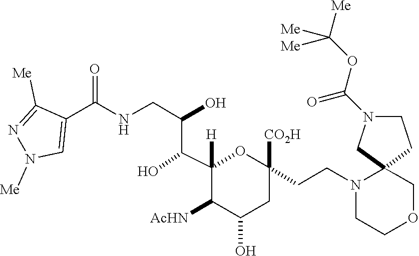 | H | 683.36 | 683.47 | 6.5 | 0.44 |
| A-137 | 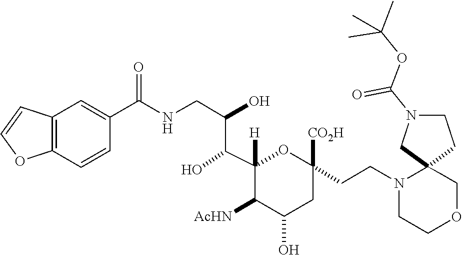 | H | 705.33 | 707.52 | 12.7 | 0.076 |
| A-138 | 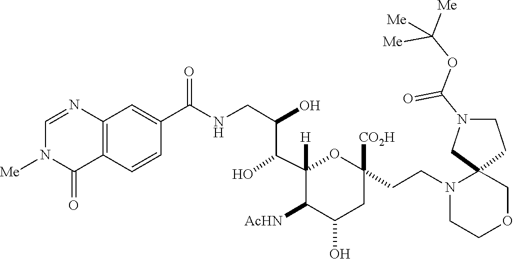 | H | 747.35 | 747.38 | 6.6 | 0.60 |
| A-139 | 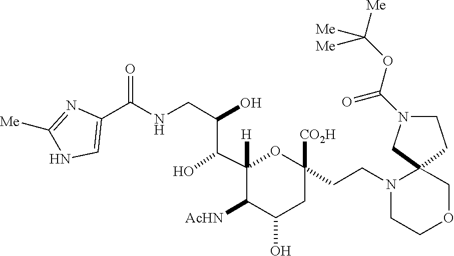 | H | 669.34 | 669.65 | 6.2 | 0.61 |
| A-140 | 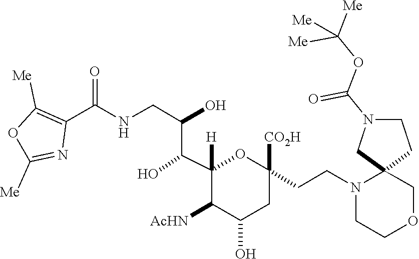 | H | 684.34 | 684.64 | 7.5 | 0.35 |
| A-141 | 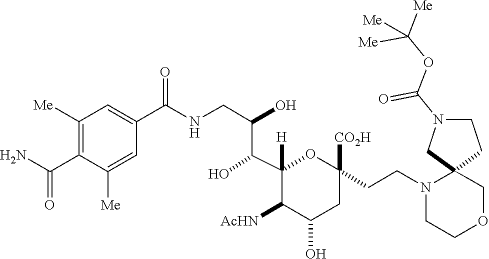 | H | 736.37 | 736.42 | 7.0 | 0.35 |
| A-142 | 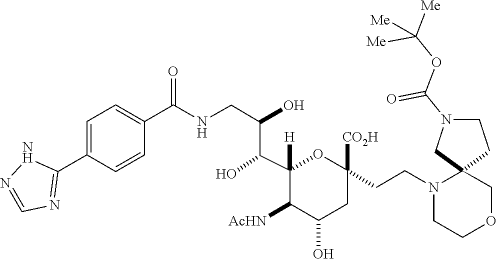 | H | 732.35 | 732.49 | 6.4 | 0.34 |
| A-143 | 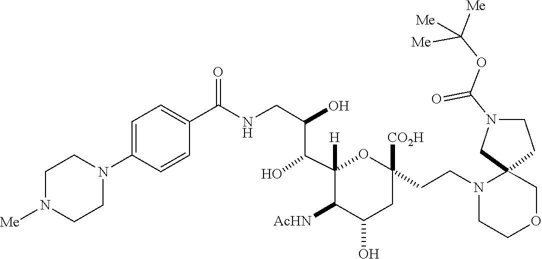 | H | 763.42 | 763.68 | 7.8 | 0.334 |
| A-144 | 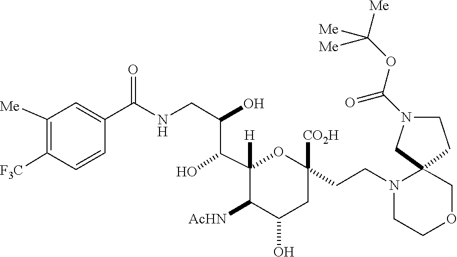 | H | 747.34 | 747.4 | 11.0 | 0.230 |
| A-145 | 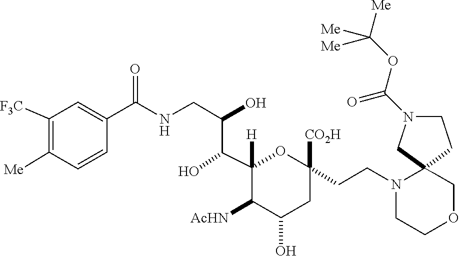 | H | 744.34 | 747.4 | 9.6 | 0.610 |
| A-146 | 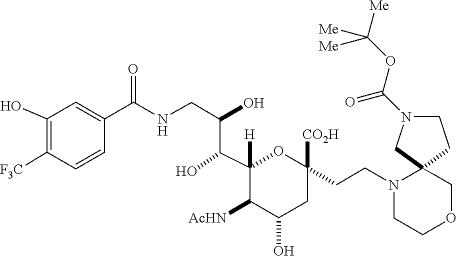 | H | 749.32 | 749.4 | 10.6 | 0.262 |
| A-147 | 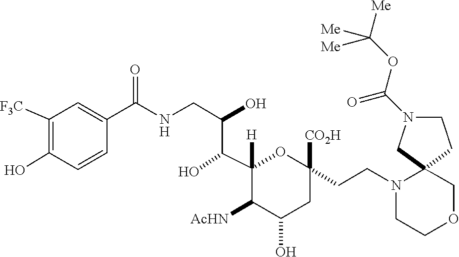 | H | 749.32 | 749.4 | 10.6 | 0.127 |
| A-148 | 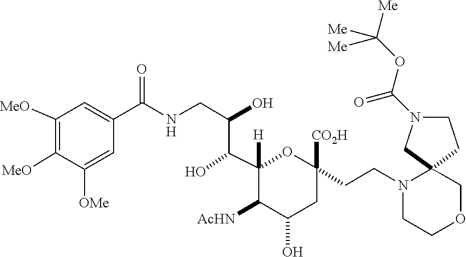 | H | 755.37 | 755.5 | 9.9 | 0.197 |
| A-149 | 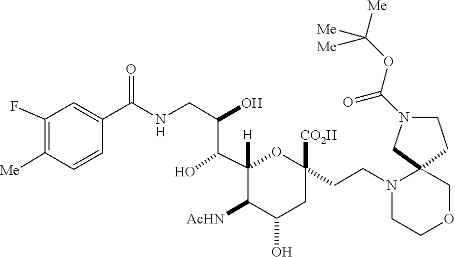 | H | 697.34 | 697.4 | 10.7 | 0.133 |
| A-150 | 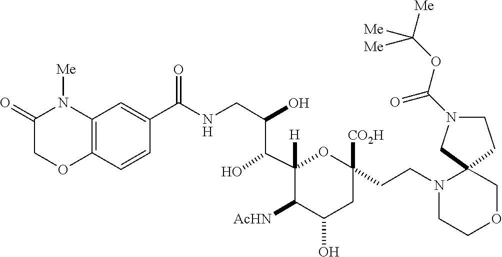 | H | 750.35 | 750.5 | 9.6 | 0.219 |
| A-151 | 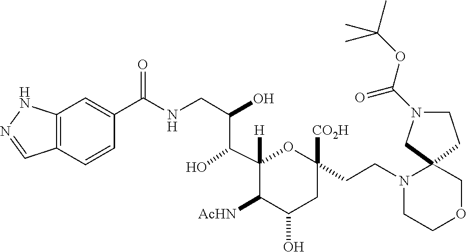 | H | 705.34 | 705.5 | 8.9 | 0.206 |
| A-152 | 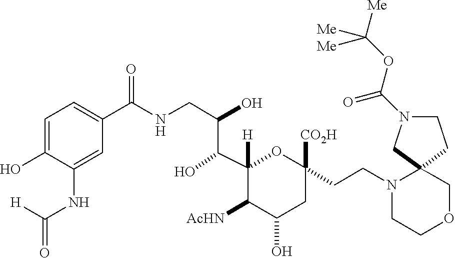 | H | 724.34 | 724.5 | 7.4 | 0.51 |
| A-153 | 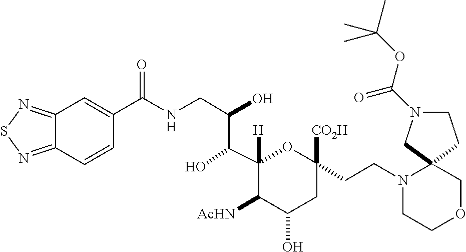 | H | 723.30 | 723.4 | 10.1 | 0.196 |
| A-154 | 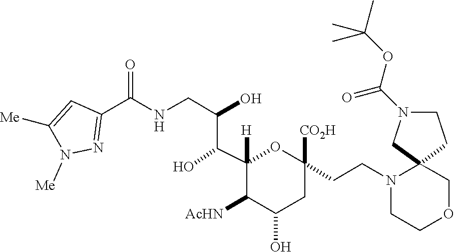 | H | 683.36 | 683.5 | 9.3 | 0.137 |
| A-155 | 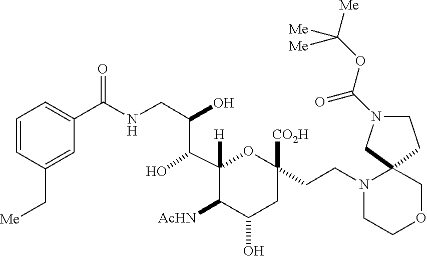 | H | 693.37 | 693.4 | 10.1 | 0.200 |
| A-156 | 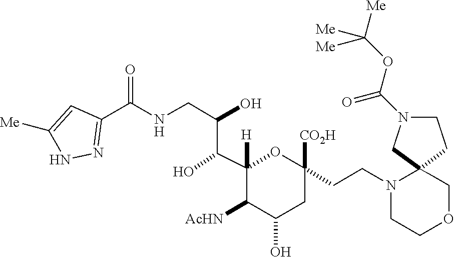 | H | 669.34 | 669.5 | 8.9 | 0.138 |
| A-157 | 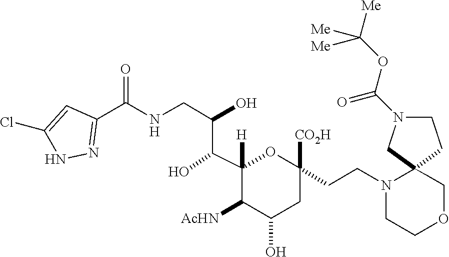 | H | 689.28 | 689.3 | 10.3 | 0.080 |
| A-158 | 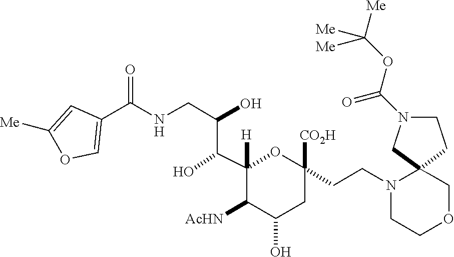 | H | 669.33 | 669.4 | 10.4 | 0.133 |
| A-159 | 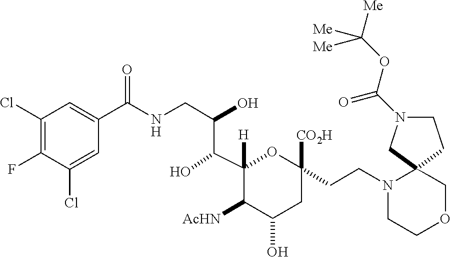 | H | 751.24 | 751.3 | 11.4 | 0.238 |
| A-160 | 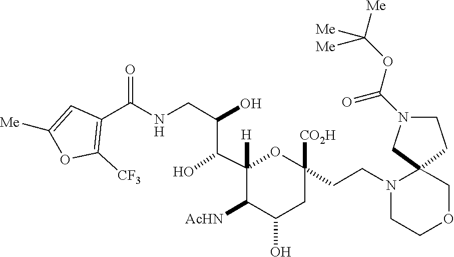 | H | 737.32 | 737.5 | 8.0 | 0.68 |
| A-161 | 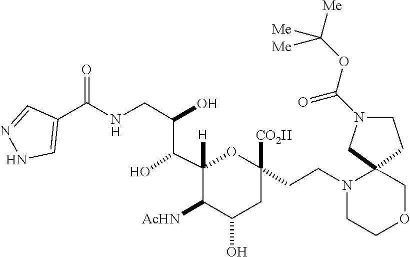 | H | 655.33 | 655.5 | 5.9 | 1.08 |
| A-162 | 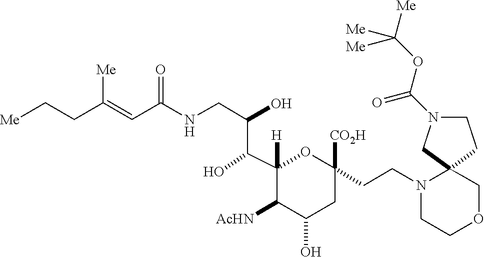 | H | 671.38 | 671.48 | 4.8 | 4.21 |
| A-163 | 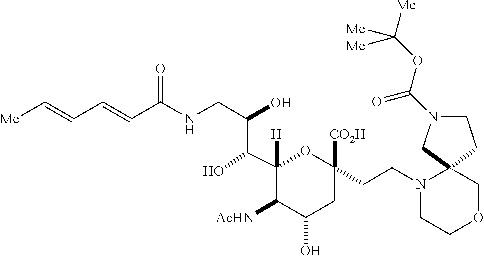 | H | 655.35 | 655.39 | 5.7 | 0.70 |
| A-164 | 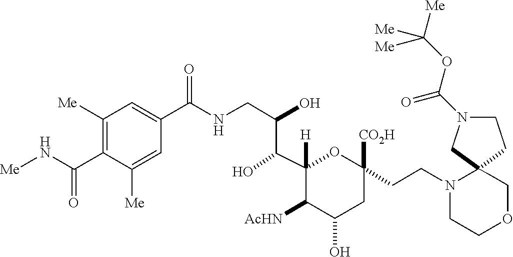 | H | 750.39 | 750.53 | 5.3 | 1.56 |
| A-165 | 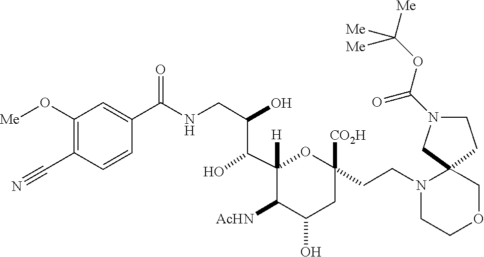 | H | 720.34 | 72054 | 5.7 | 1.14 |
| A-166 | 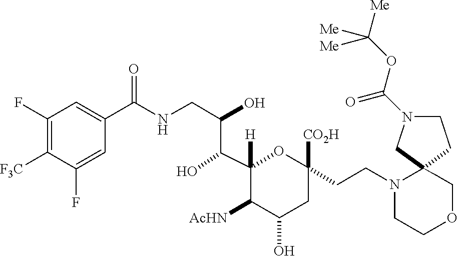 | H | 769.30 | 769.4 | 6.9 | 4.00 |
| A-167 | 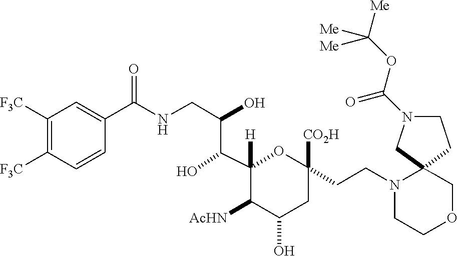 | H | 801.31 | 801.4 | 7.9 | 3.34 |
| A-168 | 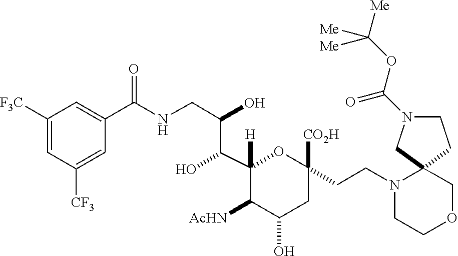 | H | 801.31 | 801.4 | 8.3 | 1.97 |
| A-169 | 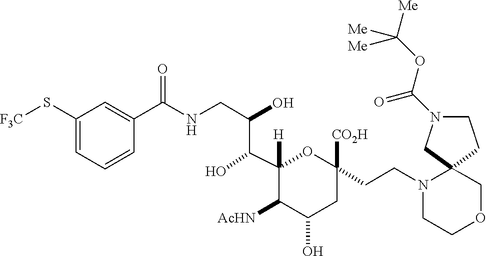 | H | 765.29 | 765.4 | 6.2 | 4.72 |
| A-170 | 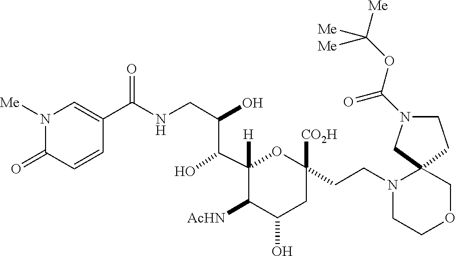 | H | 696.34 | 696.4 | 6.0 | 3.87 |
| A-171 | 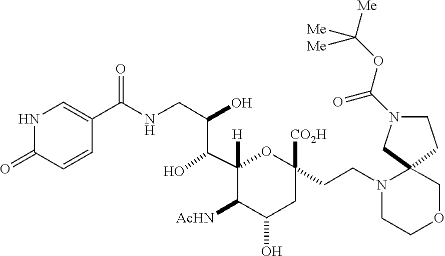 | H | 682.33 | 682.4 | 3.6 | 10.30 |
| A-172 | 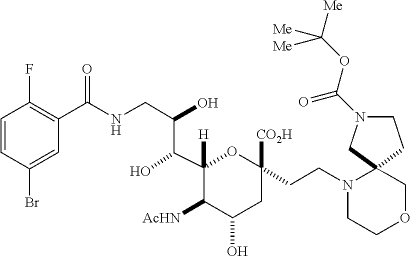 | H | 763.23 | 763.3 | 7.6 | 0.82 |
| A-173 | 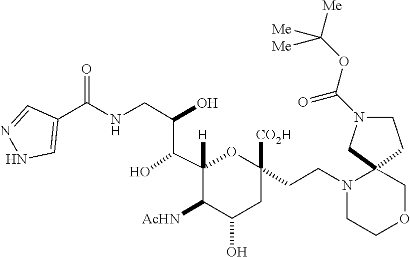 | H | 655.33 | 655.5 | 5.9 | 1.08 |
| A-174 | 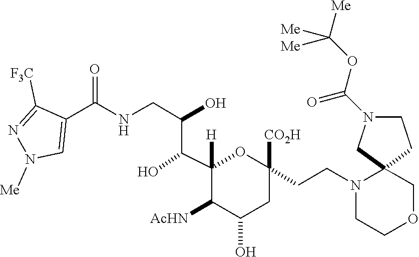 | H | 737.33 | 737.4 | 6.6 | 1.17 |
| A-175 | 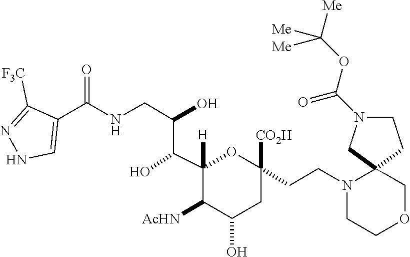 | H | 723.31 | 723.4 | 4.6 | 4.11 |
| A-176 | 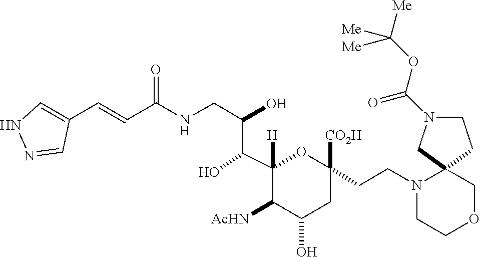 | H | 681.34 | 681.4 | | 0.65 |
| A-177 | 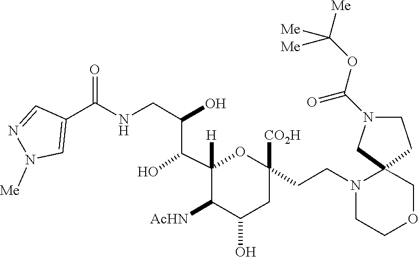 | H | 669.34 | 669.5 | 7.5 | 0.67 |
| A-178 | 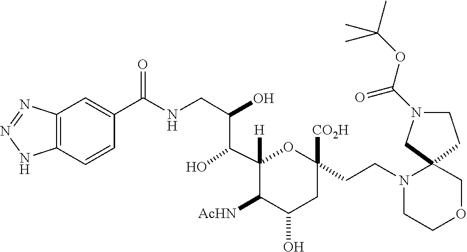 | H | 706.34 | 706.6 | 9.5 | 0.146 |
| A-179 | 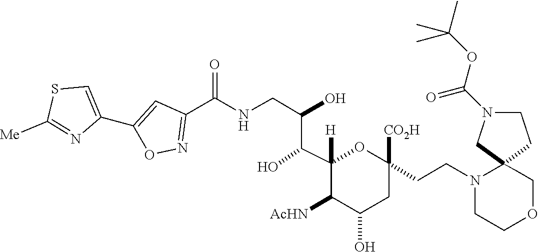 | H | 753.31 | 753.32 | 7.7 | 0.217 |
| A-180 | 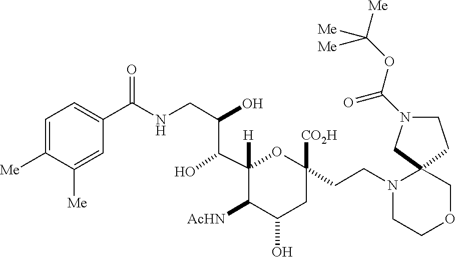 | Na | 715.37 | 715.38 | 10.9 | 0.074 |
| A-181 | 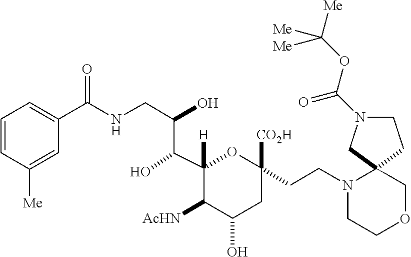 | H | 679.35 | 679.42 | 10.9 | 0.070 |
| A-182 | 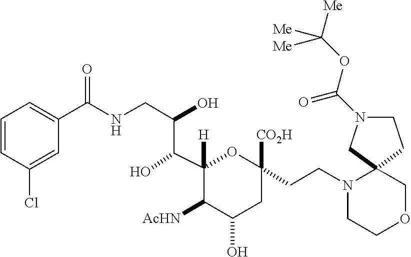 | H | 699.30 | 699.35 | 11.7 | 0.073 |
| A-183 | 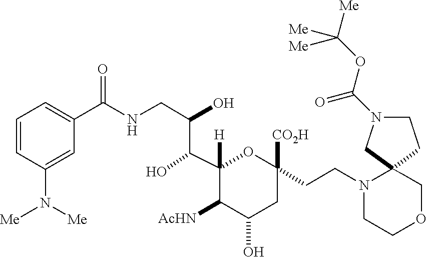 | H | 708.38 | 708.36 | 10.3 | 0.173 |
| A-184 | 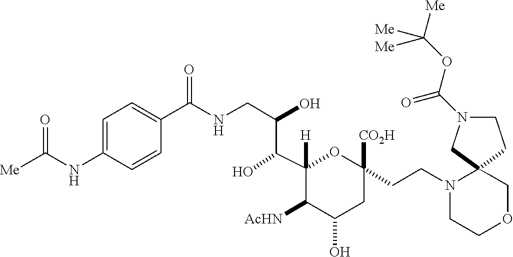 | H | 722.36 | 722.42 | 6.0 | 1.09 |
| A-185 | 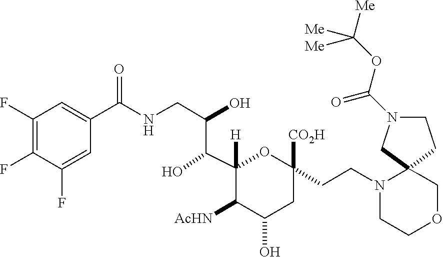 | H | 719.31 | 719.51 | 5.0 | 3.60 |
| A-186 | 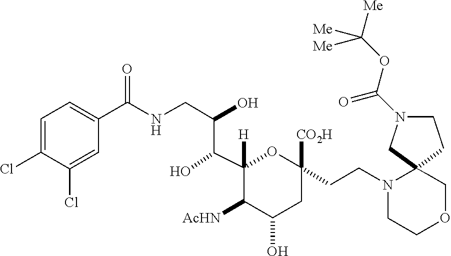 | H | 733.35 | 733.36 | 10.8 | 0.127 |
| A-187 | 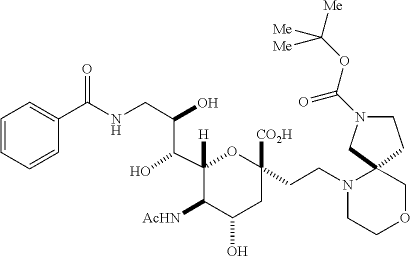 | H | 665.34 | 665.52 | 6.8 | 1.01 |
| A-188 | 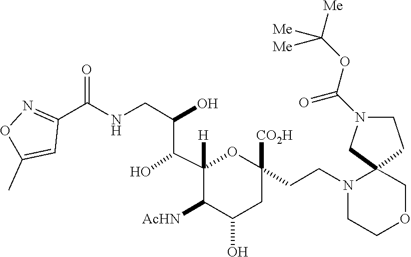 | H | 670.33 | 670.49 | 4.5 | 2.08 |
| A-189 | 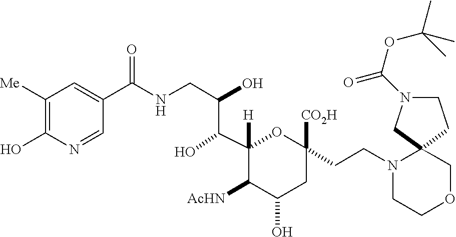 | H | 696.34 | 696.6 | 9.8 | 0.150 |
| A-190 | 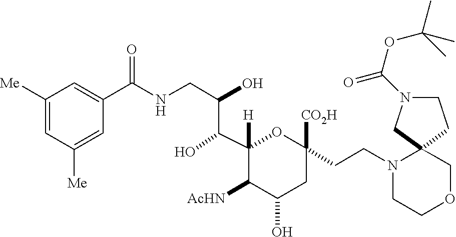 | Na | 715.36 | 715.45 | 10.1 | 0.119 |
| A-191 | 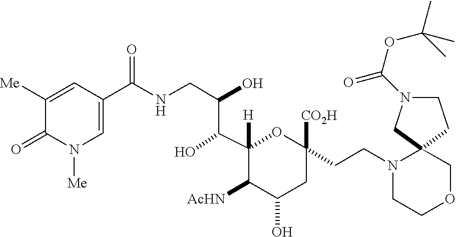 | H | 710.36 | 710.5 | 9.4 | 0.163 |
| A-192 | 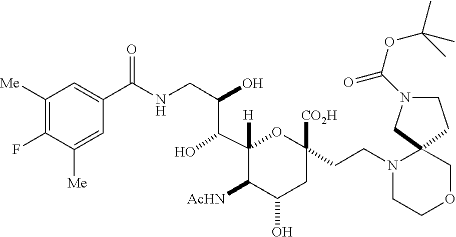 | H | 711.36 | 711.5 | 12.1 | 0.047 |
| A-193 | 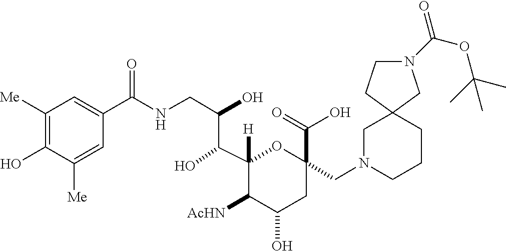 [SMILES: CC(=O)N[C@H]1[C@H]([C@H](O)[C@H](O)CNC(=O)c2cc(C)c(O)c(C)c2)O[C@@](CN2CCCC3(CCN(C(=O)OC(C)(C)C)C3)C2)(C(=O)O)C[C@@H]1O] | H | 693.37 | 693.39 | | 0.090 |
| A-194 | 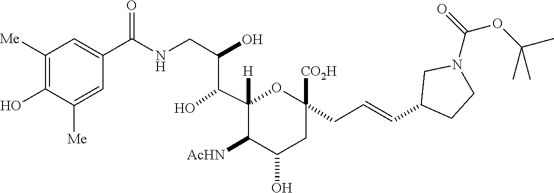 | H | 650.32 | 650.5 | 11.7 | 0.026 |
| A-195 | 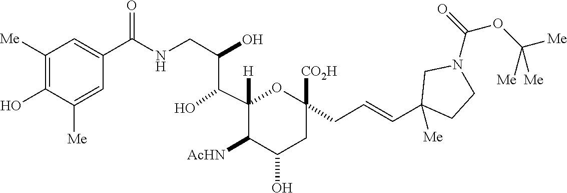 [SMILES: CC(=O)N[C@H]1[C@H]([C@H](O)[C@H](O)CNC(=O)c2cc(C)c(O)c(C)c2)O[C@@](C/C=C/C2(C)CCN(C(=O)OC(C)(C)C)C2)(C(=O)O)C[C@@H]1O] | H | 664.34 | 664.20 | 10.8 | 0.042 |
| A-196 | 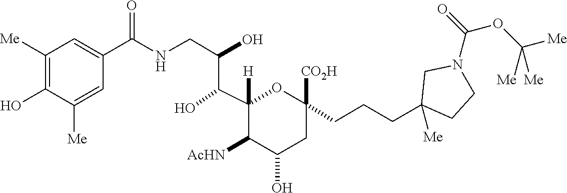 [SMILES: CC(=O)N[C@H]1[C@H]([C@H](O)[C@H](O)CNC(=O)c2cc(C)c(O)c(C)c2)O[C@@](CCCC2(C)CCN(C(=O)OC(C)(C)C)C2)(C(=O)O)C[C@@H]1O] | Na | 688.36 | 688.25 | 7.7 | 0.257 |
| A-197 | 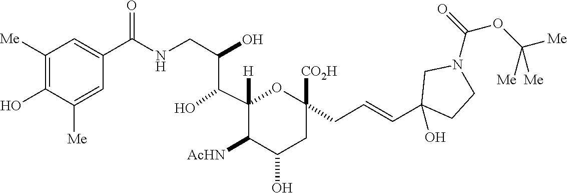 [SMILES: CC(=O)N[C@H]1[C@H]([C@H](O)[C@H](O)CNC(=O)c2cc(C)c(O)c(C)c2)O[C@@](C/C=C/C2(O)CCN(C(=O)OC(C)(C)C)C2)(C(=O)O)C[C@@H]1O] | H | 666.32 | 666.6 | | 0.181 |
| A-198 | 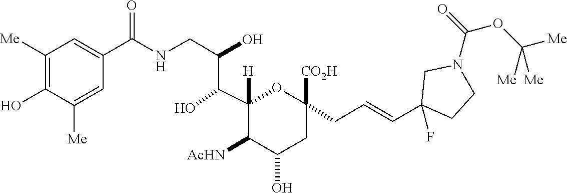 [SMILES: CC(=O)N[C@H]1[C@H]([C@H](O)[C@H](O)CNC(=O)c2cc(C)c(O)c(C)c2)O[C@@](C/C=C/C2(F)CCN(C(=O)OC(C)(C)C)C2)(C(=O)O)C[C@@H]1O] | H | 668.31 | 667.6 | | 0.148 |
| A-199 | 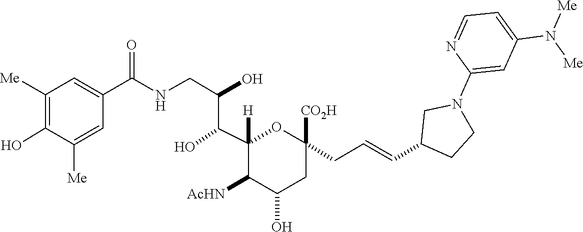 | H | 670.34 | 670.40 | | 9.86 |
| A-200 | 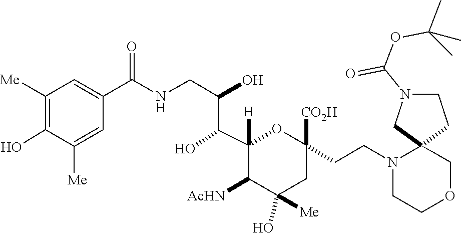 | H | 723.38 | 723.5 | 8.7 | 0.131 |
| A-201 | 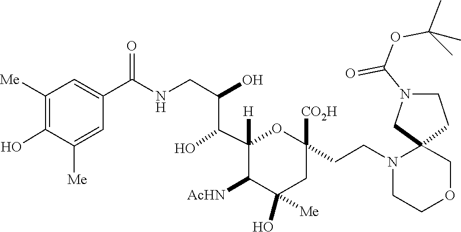 | H | 723.38 | 723.4 | 6.2 | 1.72 |
| A-202 | 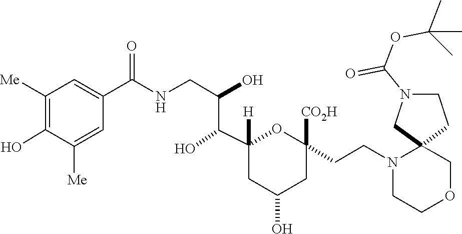 | H | 652.34 | 652.33 | 5.46 | 0.311 |
| A-203 | 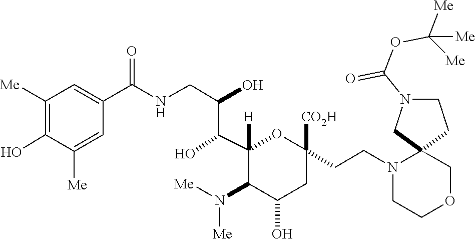 | H | 695.34 | 695.49 | | >5.0 |
| A-204 | 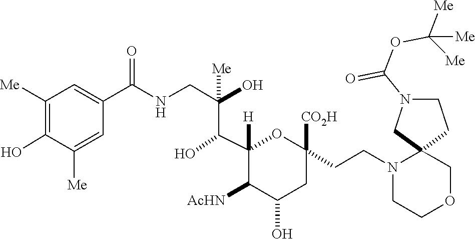 | H | 723.38 | 723.33 | 5.0 | 0.582 |
| A-205 | 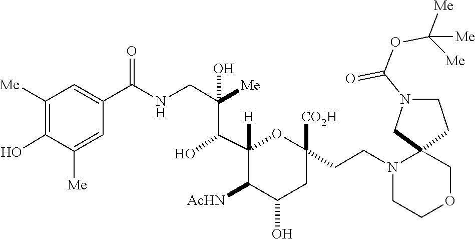 | H | 723.38 | 723.28 | 3.5 | 1.82 |
| A-206 | 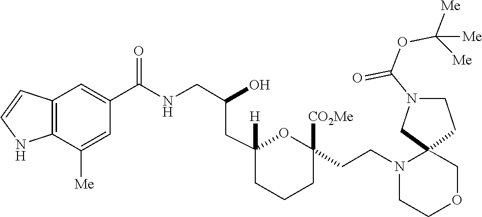 | H | 643.37 | 643.71 | 0.1 | >100 |
| A-207 | 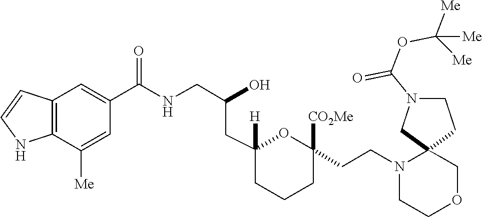 | H | 629.35 | 630.60 | 5.0 | 3.75 |
| A-208 | 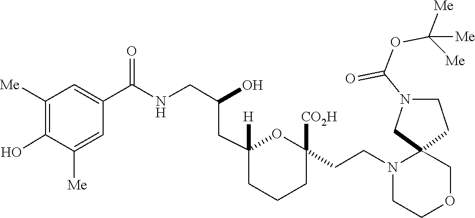 | H | 620.35 | 620.86 | | >20 |
| A-209 | 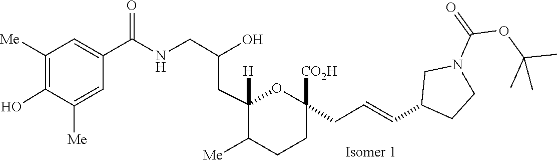 | H | 575.33 | 575.48 | | 44.5 |
| A-210 | 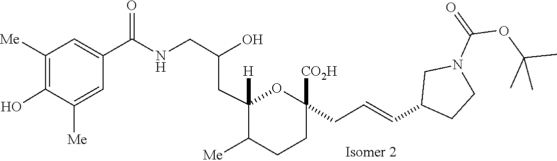 | H | 575.33 | 575.48 | | 21.4 |
| A-211 | 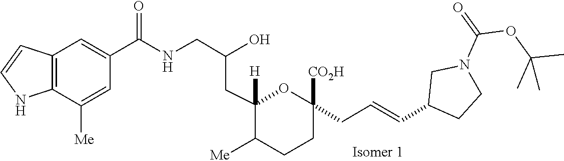 | H | 584.33 | 584.30 | | 15.2 |
| A-212 | 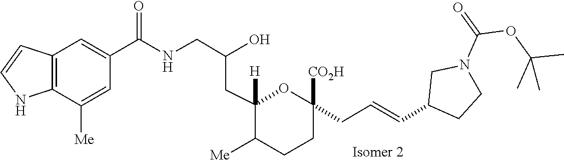 | H | 584.33 | 584.30 | | 17.4 |
| A-213 | 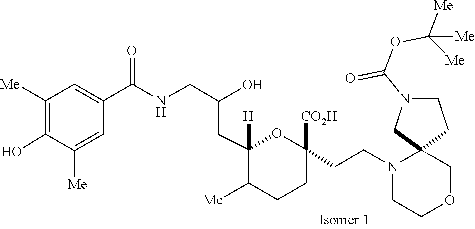 | H | 634.37 | 634.55 | | 17.9 |
| A-214 | 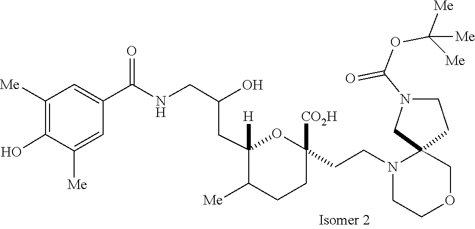 | H | 634.37 | 634.55 | | 9.8 |
| A-215 | 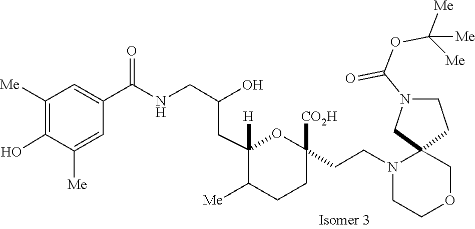 | H | 634.37 | 634.55 | | 60.4 |
| A-216 | 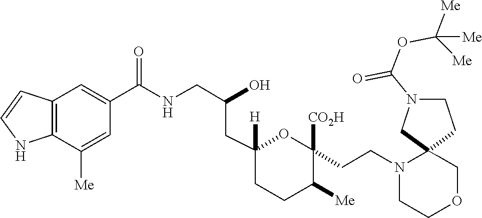 | H | 643.37 | 643.27 | | 12.2 |
| A-217 | 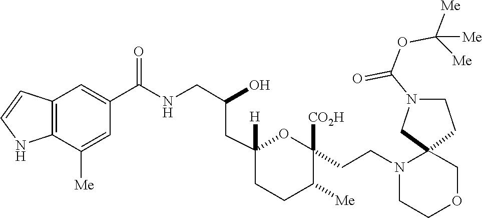 | H | 643.37 | 643.51 | | >20 |
| A-218 | 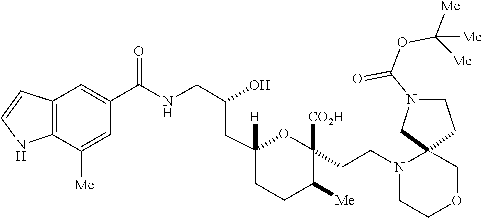 | H | 643.37 | 643.33 | | >20 |
| A-219 | 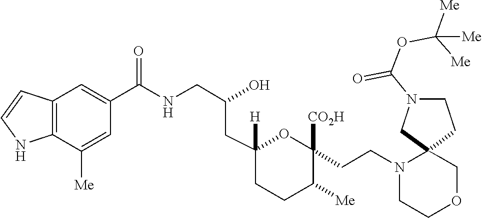 | H | 643.37 | 643.48 | | >20 |
| A-220 | 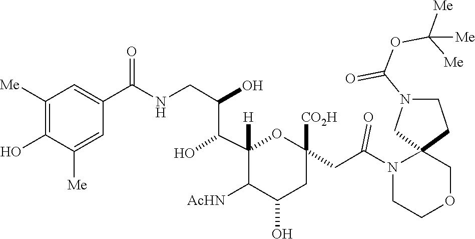 | H | 723.34 | 723.55 | 9.3 | 0.055 |
| A-221 | 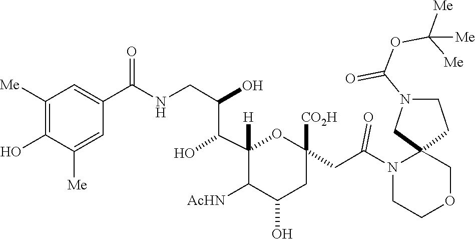 | H | 723.34 | 723.55 | 7.0 | 0.162 |
| A-222 | 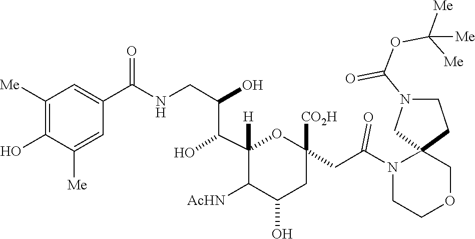 | H | 723.34 | 723.55 | 9.3 | 0.055 |
| A-223 | 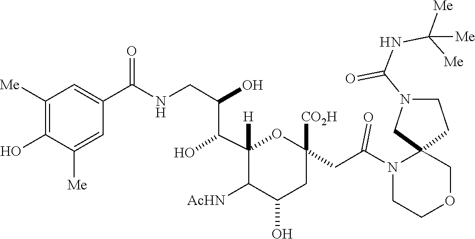 | H | 722.36 | 722.5 | 8.9 | 0.123 |
| A-224 | 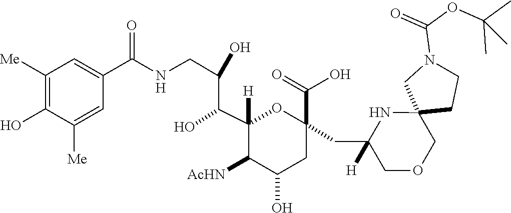 | H | 695.35 | 695.42 | 8.9 | 0.143 |
| A-225 | 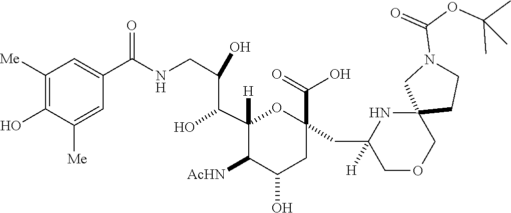 | H | 695.35 | 695.43 | 6.4 | 0.552 |
| A-226 | 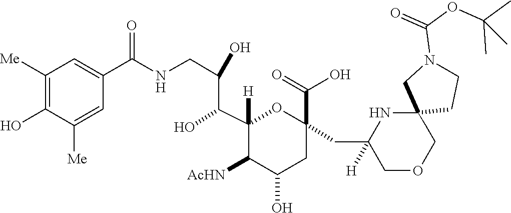 | H | 695.35 | 695.54 | | 3.87 |
| A-227 | 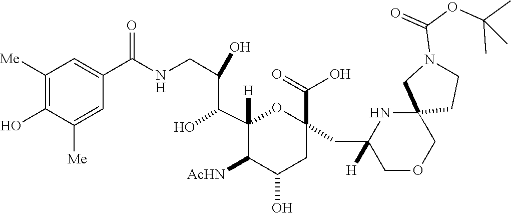 | H | 695.35 | 695.44 | 14.6 | 0.012 |
| A-228 | 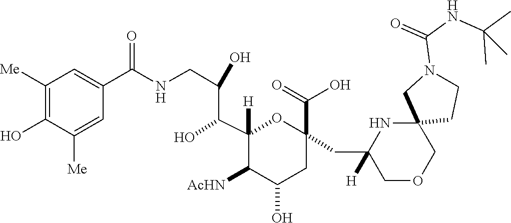 | H | 694.36 | 695.34 | 13.4 | 0.014 |
| A-229 | 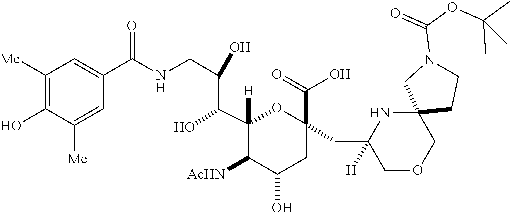 | H | 695.35 | 695.40 | 13.0 | 0.022 |
| A-230 | 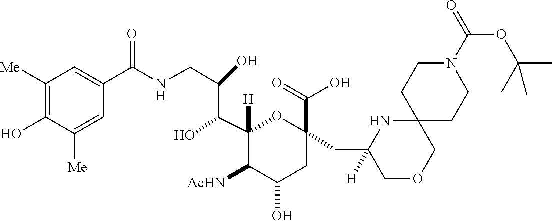 [SMILES: CC(=O)N[C@H]1[C@H]([C@H](O)[C@H](O)CNC(=O)c2cc(C)c(O)c(C)c2)O[C@@](C[C@@H]2COCC3(CCN(C(=O)OC(C)(C)C)CC3)N2)(C(=O)O)C[C@@H]1O] | H | 709.36 | 709.39 | 1.1 | 10.62 |
| A-231 | 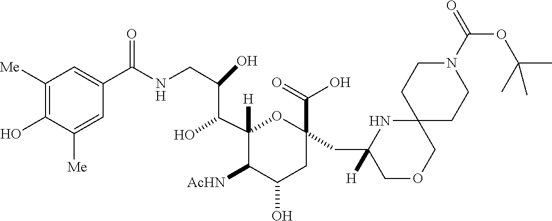 [SMILES: CC(=O)N[C@H]1[C@H]([C@H](O)[C@H](O)CNC(=O)c2cc(C)c(O)c(C)c2)O[C@@](C[C@H]2COCC3(CCN(C(=O)OC(C)(C)C)CC3)N2)(C(=O)O)C[C@@H]1O] | H | 709.36 | 709.49 | 5.2 | 1.41 |
| A-232 | 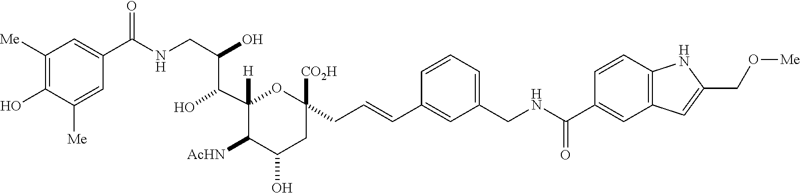 [SMILES: COCc1cc2cc(C(=O)NCc3cccc(/C=C/C[C@]4(C(=O)O)C[C@H](O)[C@@H](NC(C)=O)[C@H]([C@H](O)[C@H](O)CNC(=O)c5cc(C)c(O)c(C)c5)O4)c3)ccc2[nH]1] | H | 773.34 | 773.6 | 9.8 | 0.074 |
| A-233 | 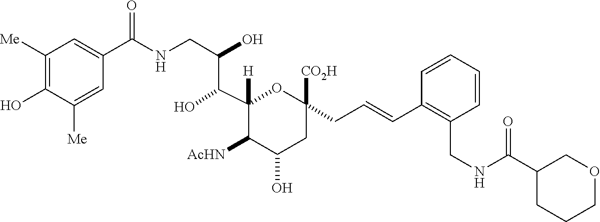 [SMILES: CC(=O)N[C@H]1[C@H]([C@H](O)[C@H](O)CNC(=O)c2cc(C)c(O)c(C)c2)O[C@@](C/C=C/c2ccccc2CNC(=O)C2CCCOC2)(C(=O)O)C[C@@H]1O] | H | 698.32 | 698.30 | 3.0 | 8.99 |
| A-234 | 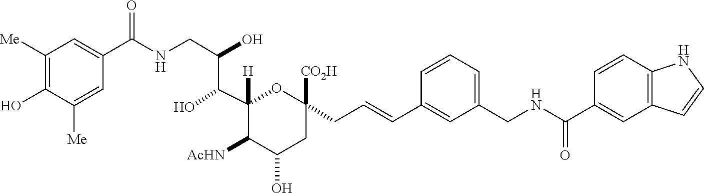 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]ccc4c3)c2)(C(=O)O)C[C@@H]1O] | H | 730.30 | 730.4 | 4.6 | 0.93 |
| A-235 | 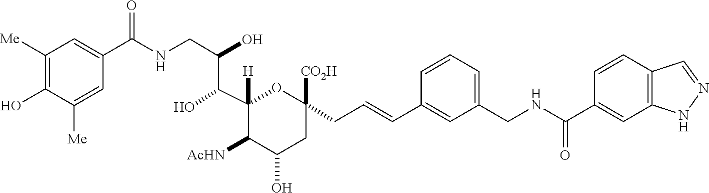 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4cn[nH]c4c3)c2)(C(=O)O)C[C@@H]1O] | H | 730.30 | 730.4 | 3.3 | 2.09 |
| A-236 | 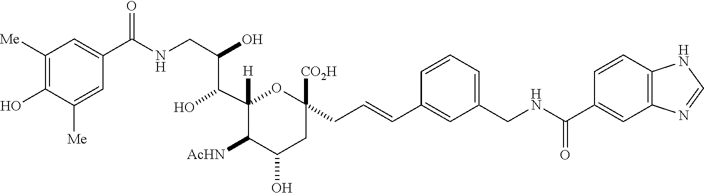 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]cnc4c3)c2)(C(=O)O)C[C@@H]1O] | H | 730.30 | 730.4 | 4.4 | 2.01 |
| A-237 | 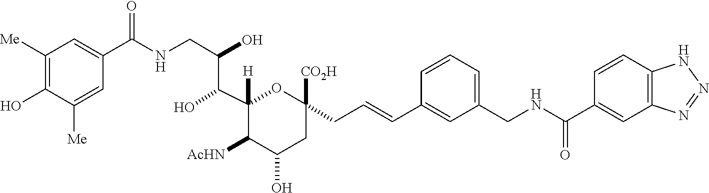 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]nnc4c3)c2)(C(=O)O)C[C@@H]1O] | H | 731.31 | 731.4 | 0.8 | 5.03 |
| A-238 | 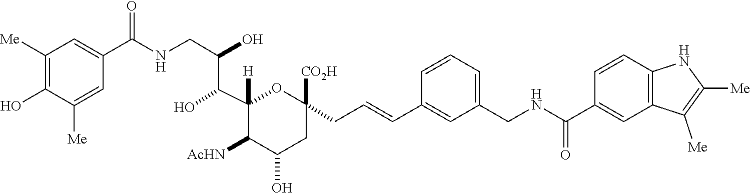 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]c(C)c(C)c4c3)c2)(C(=O)O)C[C@@H]1O] | H | 757.35 | 757.4 | 9.2 | 0.347 |
| A-239 | 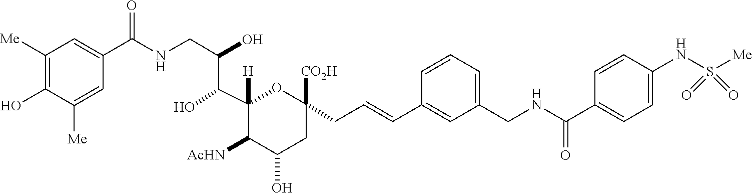 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc(NS(C)(=O)=O)cc3)c2)(C(=O)O)C[C@@H]1O] | H | 783.29 | 783.4 | 2.2 | 1.12 |
| A-240 | 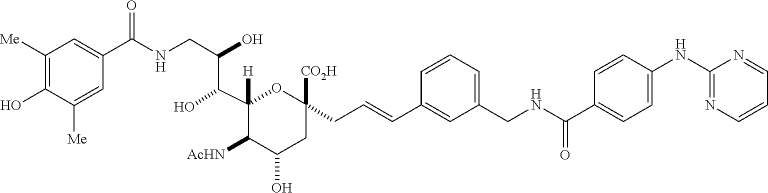 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc(Nc4ncccn4)cc3)c2)(C(=O)O)C[C@@H]1O] | H | 783.33 | 783.4 | 6.2 | 0.227 |
| A-241 | 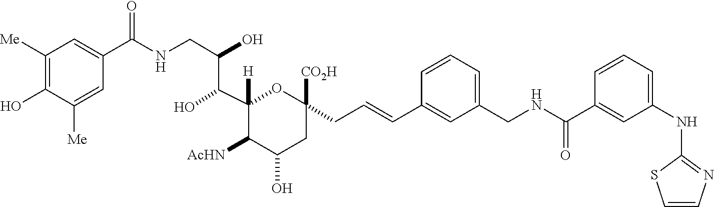 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3cccc(Nc4nccs4)c3)c2)(C(=O)O)C[C@@H]1O] | | 788.29 | 788.3 | 5.3 | 7.97 |
| A-242 | 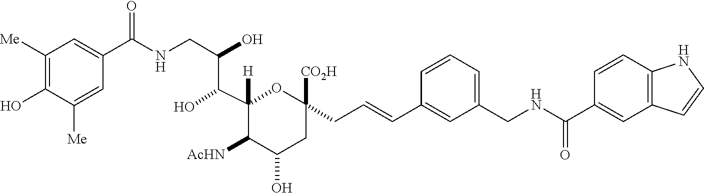 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]ccc4c3)c2)(C(=O)O)C[C@@H]1O] | | 729.31 | 729.4 | 7.6 | 0.502 |
| A-243 | 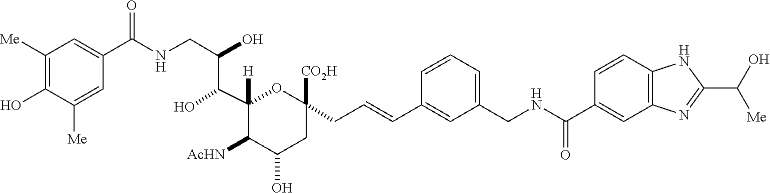 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]c(C(C)O)nc4c3)c2)(C(=O)O)C[C@@H]1O] | | 774.33 | 774.4 | 5.4 | 0.255 |
| A-244 | 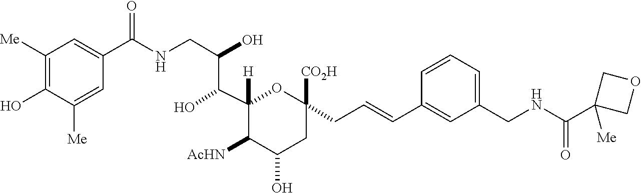 [SMILES: CC(=O)N[C@H]1[C@H]([C@H](O)[C@H](O)CNC(=O)c2cc(C)c(O)c(C)c2)O[C@@](C/C=C/c2cccc(CNC(=O)C3(C)COC3)c2)(C(=O)O)C[C@@H]1O] | | 684.31 | 684.4 | 0.8 | 9.56 |
| A-245 | 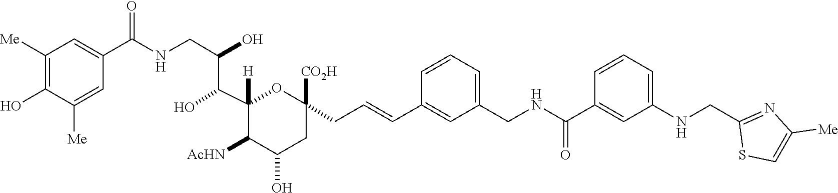 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3cccc(NCc4nc(C)cs4)c3)c2)(C(=O)O)C[C@@H]1O] | H | 816.32 | 816.4 | 1.2 | 9.46 |
| A-246 | 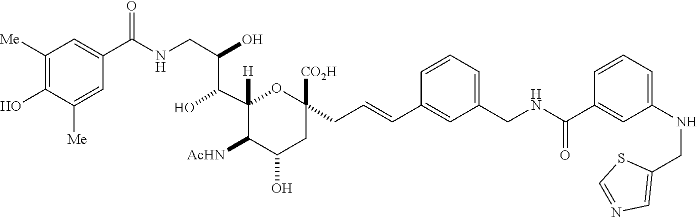 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3cccc(NCc4cncs4)c3)c2)(C(=O)O)C[C@@H]1O] | H | 802.31 | 802.4 | 2.2 | 4.00 |
| A-247 | 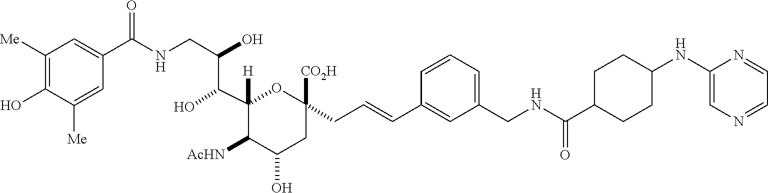 [SMILES: CC(=O)N[C@H]1[C@H]([C@H](O)[C@H](O)CNC(=O)c2cc(C)c(O)c(C)c2)O[C@@](C/C=C/c2cccc(CNC(=O)C3CCC(Nc4cnccn4)CC3)c2)(C(=O)O)C[C@@H]1O] | H | 789.38 | 789.6 | 8.5 | 0.353 |
| A-248 | 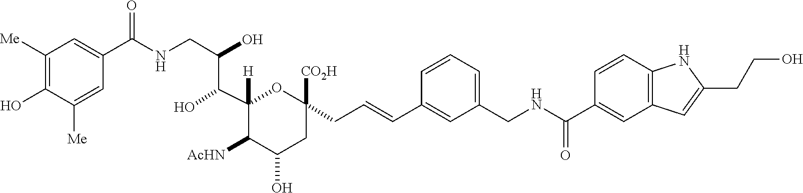 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]c(CCO)cc4c3)c2)(C(=O)O)C[C@@H]1O] | H | 773.34 | 774.6 | 6.0 | 0.682 |
| A-249 | 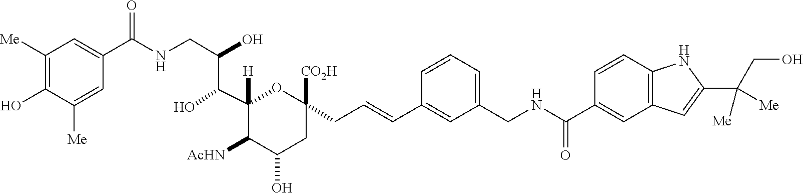 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]c(C(C)(C)CO)cc4c3)c2)(C(=O)O)C[C@@H]1O] | H | 802.37 | 802.7 | 8.1 | 0.224 |
| A-250 | 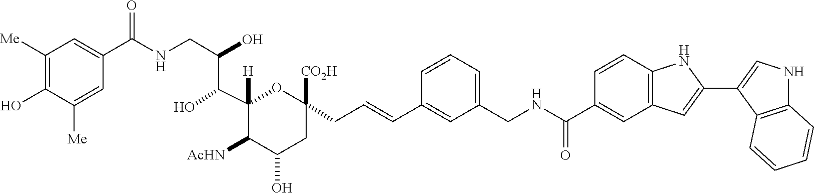 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]c(-c5c[nH]c6ccccc56)cc4c3)c2)(C(=O)O)C[C@@H]1O] | H | 845.35 | 845.7 | 8.4 | 3.26 |
| A-251 | 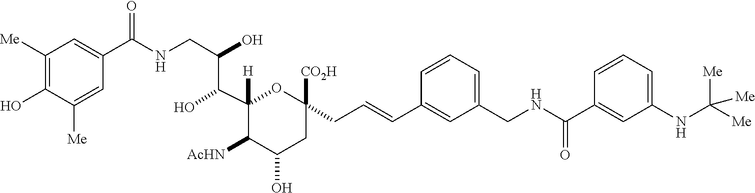 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3cccc(NC(C)(C)C)c3)c2)(C(=O)O)C[C@@H]1O] | H | 761.37 | 761.6 | 4.3 | 9.79 |
| A-252 | 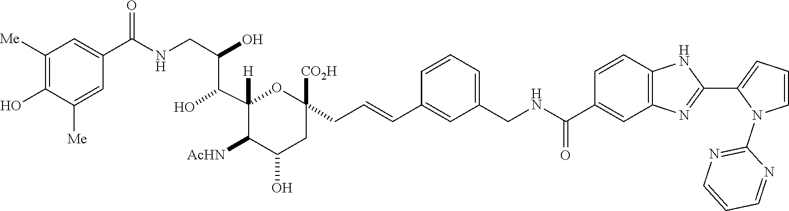 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]c(-c5cccn5-c5ncccn5)nc4c3)c2)(C(=O)O)C[C@@H]1O] | H | 873.35 | 873.7 | 7.6 | 0.578 |
| A-253 | 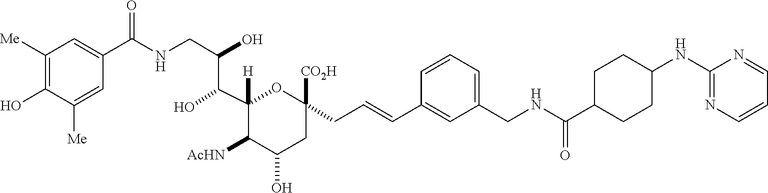 [SMILES: CC(=O)N[C@H]1[C@H]([C@H](O)[C@H](O)CNC(=O)c2cc(C)c(O)c(C)c2)O[C@@](C/C=C/c2cccc(CNC(=O)C3CCC(Nc4ncccn4)CC3)c2)(C(=O)O)C[C@@H]1O] | H | 789.38 | 789.6 | 9.6 | 0.195 |
| A-254 | 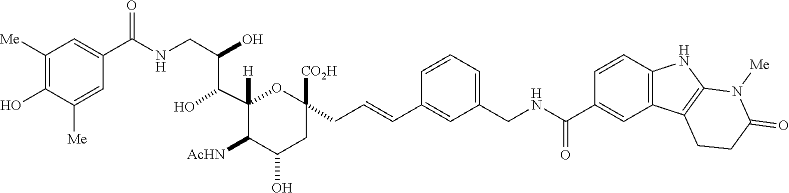 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc4[nH]c5c(c4c3)CCC(=O)N5C)c2)(C(=O)O)C[C@@H]1O] | H | 812.35 | 812.7 | 9.2 | 0.193 |
| A-255 | 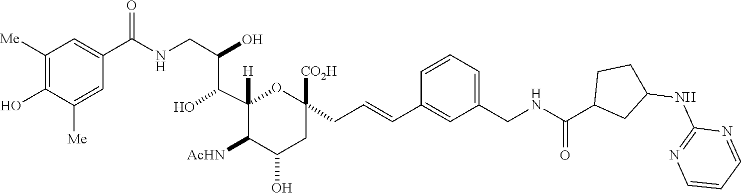 [SMILES: CC(=O)N[C@H]1[C@H]([C@H](O)[C@H](O)CNC(=O)c2cc(C)c(O)c(C)c2)O[C@@](C/C=C/c2cccc(CNC(=O)C3CCC(Nc4ncccn4)C3)c2)(C(=O)O)C[C@@H]1O] | H | 775.36 | 775.6 | 4.2 | 4.46 |
| A-256 | 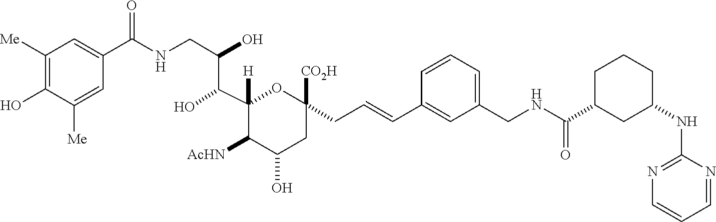 | H | 789.38 | 789.7 | 5.1 | 2.17 |
| A-257 | 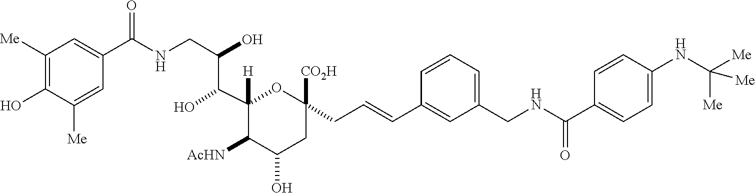 [SMILES: CC(=O)N[C@H]1[C@H]([C@H](O)[C@H](O)CNC(=O)c2cc(C)c(O)c(C)c2)O[C@@](C/C=C/c2cccc(CNC(=O)c3ccc(NC(C)(C)C)cc3)c2)(C(=O)O)C[C@@H]1O] | H | 761.37 | 761.6 | 5.9 | 1.60 |
| A-258 | 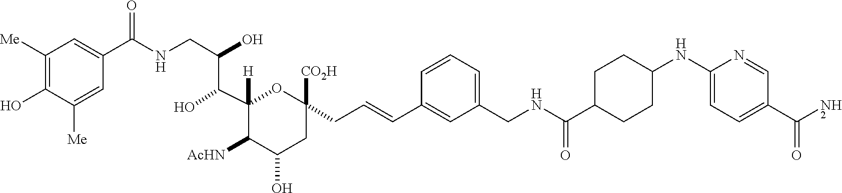 [SMILES: CC(=O)N[C@H]1[C@H]([C@H](O)[C@H](O)CNC(=O)c2cc(C)c(O)c(C)c2)O[C@@](C/C=C/c2cccc(CNC(=O)C3CCC(Nc4ccc(C(N)=O)cn4)CC3)c2)(C(=O)O)C[C@@H]1O] | H | 831.39 | 831.7 | 0.8 | 9.37 |
| A-259 | 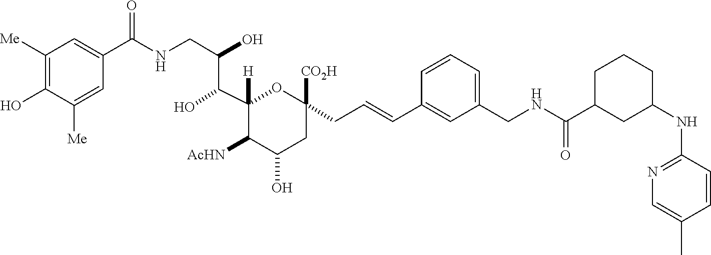 [SMILES: CC(=O)N[C@H]1[C@H]([C@H](O)[C@H](O)CNC(=O)c2cc(C)c(O)c(C)c2)O[C@@](C/C=C/c2cccc(CNC(=O)C3CCCC(Nc4ccc(C)cn4)C3)c2)(C(=O)O)C[C@@H]1O] | H | 802.40 | 802.6 | 3.1 | 5.81 |
| A-260 | 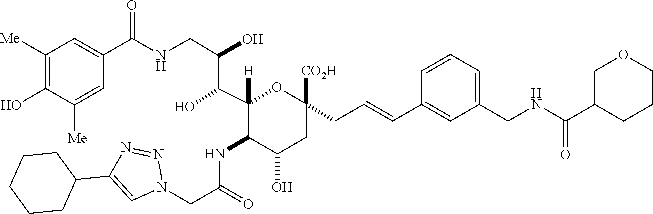 [SMILES: Cc1cc(C(=O)NC[C@@H](O)[C@@H](O)[C@@H]2O[C@@](C/C=C/c3cccc(CNC(=O)C4CCCOC4)c3)(C(=O)O)C[C@H](O)[C@H]2NC(=O)Cn2cc(C3CCCCC3)nn2)cc(C)c1O] | H | 847.42 | 847.5 | 4.2 | 7.94 |
| A-261 | 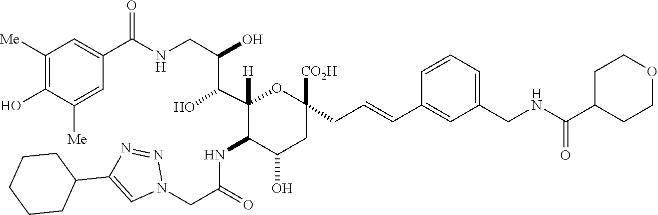 [SMILES: Cc1cc(C(=O)NC[C@@H](O)[C@@H](O)[C@@H]2O[C@@](C/C=C/c3cccc(CNC(=O)C4CCOCC4)c3)(C(=O)O)C[C@H](O)[C@H]2NC(=O)Cn2cc(C3CCCCC3)nn2)cc(C)c1O] | H | 847.42 | 847.7 | 4.4 | 1.44 |
| A-262 | 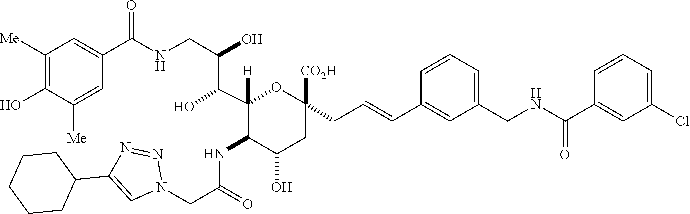 [SMILES: Cc1cc(C(=O)NC[C@@H](O)[C@@H](O)[C@@H]2O[C@@](C/C=C/c3cccc(CNC(=O)c4cccc(Cl)c4)c3)(C(=O)O)C[C@H](O)[C@H]2NC(=O)Cn2cc(C3CCCCC3)nn2)cc(C)c1O] | H | 873.35 | 873.6 | 1.9 | 6.03 |
| A-263 | 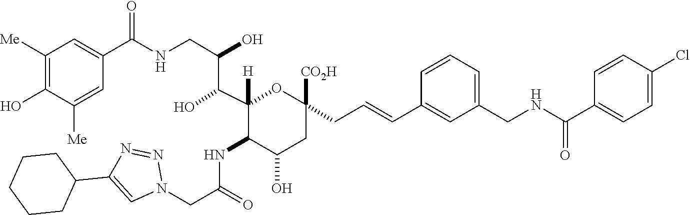 [SMILES: Cc1cc(C(=O)NC[C@@H](O)[C@@H](O)[C@@H]2O[C@@](C/C=C/c3cccc(CNC(=O)c4ccc(Cl)cc4)c3)(C(=O)O)C[C@H](O)[C@H]2NC(=O)Cn2cc(C3CCCCC3)nn2)cc(C)c1O] | H | 873.35 | 873.6 | 3.5 | 4.00 |
| A-264 | 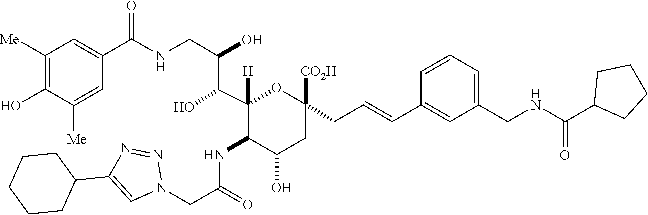 [SMILES: Cc1cc(C(=O)NC[C@@H](O)[C@@H](O)[C@@H]2O[C@@](C/C=C/c3cccc(CNC(=O)C4CCCC4)c3)(C(=O)O)C[C@H](O)[C@H]2NC(=O)Cn2cc(C3CCCCC3)nn2)cc(C)c1O] | H | 831.42 | 831.6 | 5.5 | 6.33 |
| A-265 | 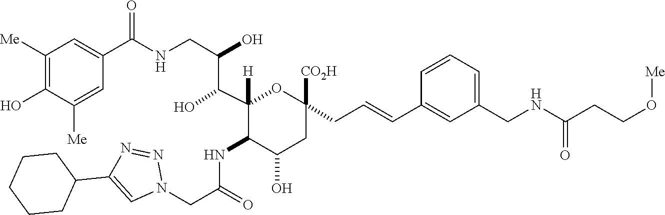 [SMILES: COCCC(=O)NCc1cccc(/C=C/C[C@]2(C(=O)O)C[C@H](O)[C@@H](NC(=O)Cn3cc(C4CCCCC4)nn3)[C@H]([C@H](O)[C@H](O)CNC(=O)c3cc(C)c(O)c(C)c3)O2)c1] | H | 821.40 | 821.6 | 4.1 | 1.37 |
| A-266 | 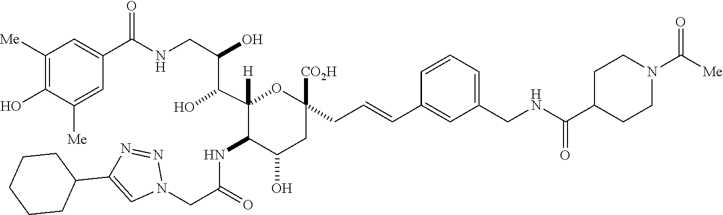 [SMILES: CC(=O)N1CCC(C(=O)NCc2cccc(/C=C/C[C@]3(C(=O)O)C[C@H](O)[C@@H](NC(=O)Cn4cc(C5CCCCC5)nn4)[C@H]([C@H](O)[C@H](O)CNC(=O)c4cc(C)c(O)c(C)c4)O3)c2)CC1] | H | 888.45 | 888.7 | 6.2 | 1.38 |
| A-267 | 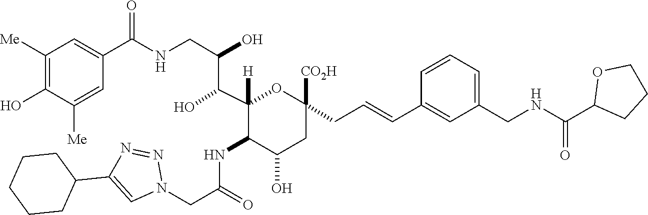 [SMILES: Cc1cc(C(=O)NC[C@@H](O)[C@@H](O)[C@@H]2O[C@@](C/C=C/c3cccc(CNC(=O)C4CCCO4)c3)(C(=O)O)C[C@H](O)[C@H]2NC(=O)Cn2cc(C3CCCCC3)nn2)cc(C)c1O] | H | 833.41 | 833.6 | 3.8 | 9.17 |
| A-268 | 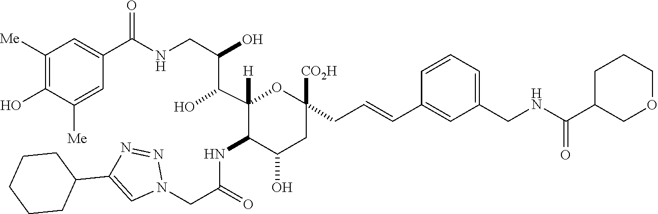 [SMILES: Cc1cc(C(=O)NC[C@@H](O)[C@@H](O)[C@@H]2O[C@@](C/C=C/c3cccc(CNC(=O)C4CCCOC4)c3)(C(=O)O)C[C@H](O)[C@H]2NC(=O)Cn2cc(C3CCCCC3)nn2)cc(C)c1O] | H | 847.42 | 847.6 | 6.2 | 1.32 |
| A-269 | 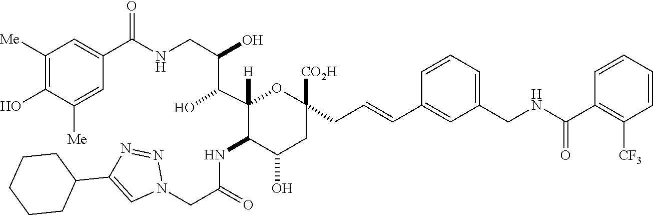 [SMILES: Cc1cc(C(=O)NC[C@@H](O)[C@@H](O)[C@@H]2O[C@@](C/C=C/c3cccc(CNC(=O)c4ccccc4C(F)(F)F)c3)(C(=O)O)C[C@H](O)[C@H]2NC(=O)Cn2cc(C3CCCCC3)nn2)cc(C)c1O] | H | 907.38 | 907.7 | 6.5 | 3.46 |
| A-270 | 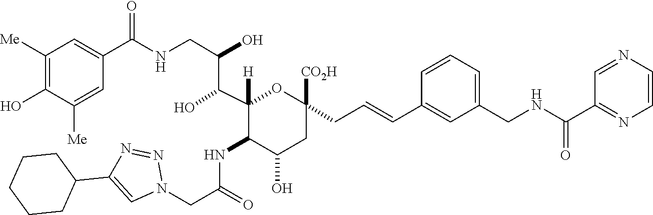 [SMILES: Cc1cc(C(=O)NC[C@@H](O)[C@@H](O)[C@@H]2O[C@@](C/C=C/c3cccc(CNC(=O)c4cnccn4)c3)(C(=O)O)C[C@H](O)[C@H]2NC(=O)Cn2cc(C3CCCCC3)nn2)cc(C)c1O] | H | 841.38 | 841.6 | 6.0 | 2.80 |
| A-271 | 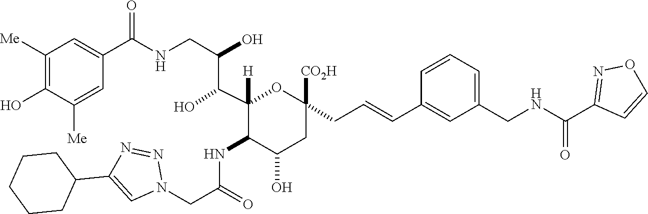 [SMILES: Cc1cc(C(=O)NC[C@@H](O)[C@@H](O)[C@@H]2O[C@@](C/C=C/c3cccc(CNC(=O)c4ccon4)c3)(C(=O)O)C[C@H](O)[C@H]2NC(=O)Cn2cc(C3CCCCC3)nn2)cc(C)c1O] | H | 830.37 | 830.5 | 5.1 | 3.99 |
| A-272 | 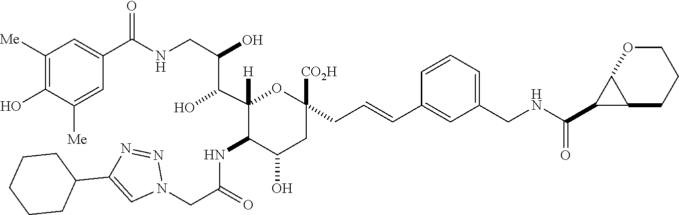 | H | 859.42 | 859.4 | 4.9 | 9.64 |
| A-273 | 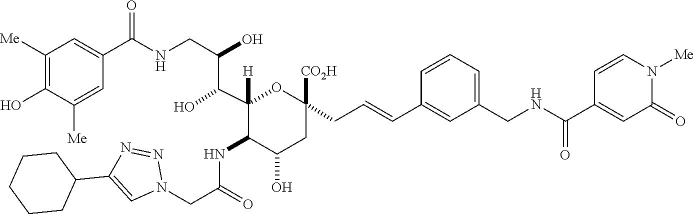 [SMILES: Cc1cc(C(=O)NC[C@@H](O)[C@@H](O)[C@@H]2O[C@@](C/C=C/c3cccc(CNC(=O)c4ccn(C)c(=O)c4)c3)(C(=O)O)C[C@H](O)[C@H]2NC(=O)Cn2cc(C3CCCCC3)nn2)cc(C)c1O] | H | 870.40 | 870.33 | 5.4 | 9.94 |
| A-274 | 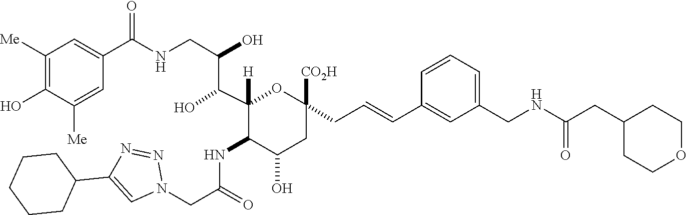 [SMILES: Cc1cc(C(=O)NC[C@@H](O)[C@@H](O)[C@@H]2O[C@@](C/C=C/c3cccc(CNC(=O)CC4CCOCC4)c3)(C(=O)O)C[C@H](O)[C@H]2NC(=O)Cn2cc(C3CCCCC3)nn2)cc(C)c1O] | H | 861.44 | 861.4 | 5.7 | 4.70 |
| A-275 | 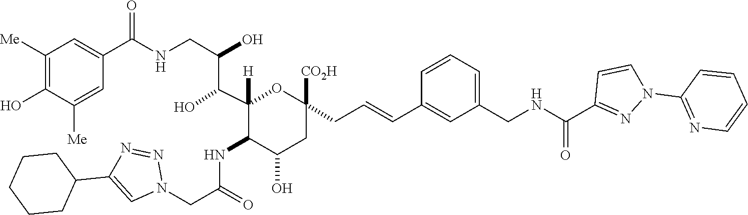 [SMILES: Cc1cc(C(=O)NC[C@@H](O)[C@@H](O)[C@@H]2O[C@@](C/C=C/c3cccc(CNC(=O)c4ccn(-c5ccccn5)n4)c3)(C(=O)O)C[C@H](O)[C@H]2NC(=O)Cn2cc(C3CCCCC3)nn2)cc(C)c1O] | H | 907.41 | 907.4 | 7.0 | 8.63 |
| A-276 | 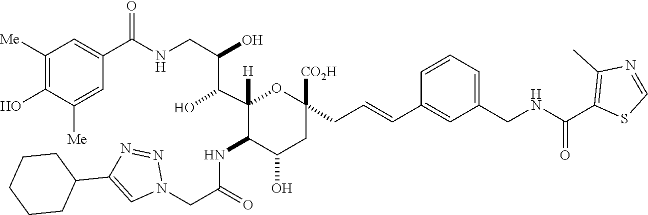 [SMILES: Cc1cc(C(=O)NC[C@@H](O)[C@@H](O)[C@@H]2O[C@@](C/C=C/c3cccc(CNC(=O)c4scnc4C)c3)(C(=O)O)C[C@H](O)[C@H]2NC(=O)Cn2cc(C3CCCCC3)nn2)cc(C)c1O] | H | 860.36 | 860.6 | 6.2 | 2.92 |
| A-277 | 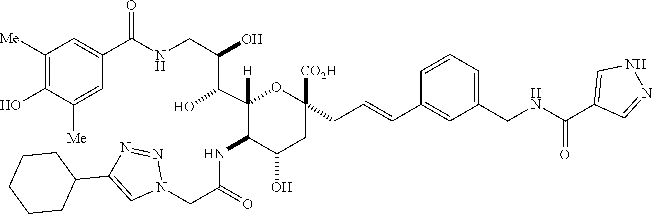 [SMILES: Cc1cc(C(=O)NC[C@@H](O)[C@@H](O)[C@@H]2O[C@@](C/C=C/c3cccc(CNC(=O)c4cn[nH]c4)c3)(C(=O)O)C[C@H](O)[C@H]2NC(=O)Cn2cc(C3CCCCC3)nn2)cc(C)c1O] | H | 829.38 | 829.5 | 4.3 | 10.24 |
| A-278 | 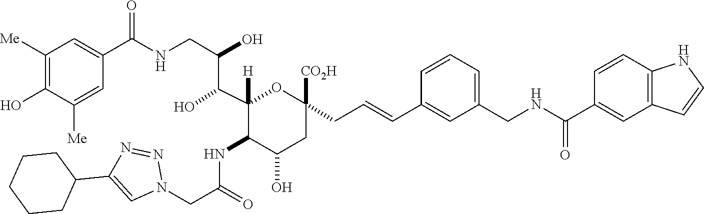 [SMILES: Cc1cc(C(=O)NC[C@@H](O)[C@@H](O)[C@@H]2O[C@@](C/C=C/c3cccc(CNC(=O)c4ccc5[nH]ccc5c4)c3)(C(=O)O)C[C@H](O)[C@H]2NC(=O)Cn2cc(C3CCCCC3)nn2)cc(C)c1O] | H | 878.40 | 878.5 | 8.8 | 1.74 |
| A-279 | 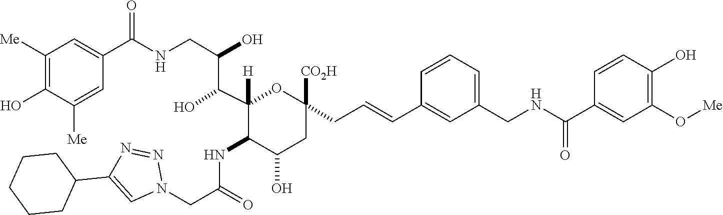 [SMILES: COc1cc(C(=O)NCc2cccc(/C=C/C[C@]3(C(=O)O)C[C@H](O)[C@@H](NC(=O)Cn4cc(C5CCCCC5)nn4)[C@H]([C@H](O)[C@H](O)CNC(=O)c4cc(C)c(O)c(C)c4)O3)c2)ccc1O] | H | 885.40 | 885.6 | 4.5 | 10.36 |
| A-280 | 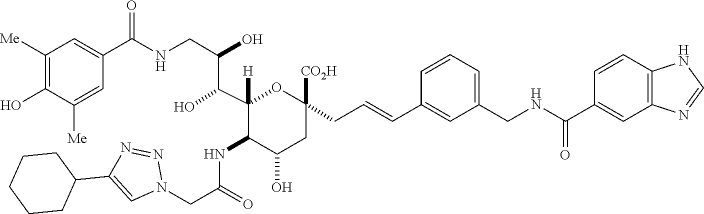 [SMILES: Cc1cc(C(=O)NC[C@@H](O)[C@@H](O)[C@@H]2O[C@@](C/C=C/c3cccc(CNC(=O)c4ccc5[nH]cnc5c4)c3)(C(=O)O)C[C@H](O)[C@H]2NC(=O)Cn2cc(C3CCCCC3)nn2)cc(C)c1O] | H | 879.40 | 879.6 | 3.8 | 4.90 |
| A-281 | 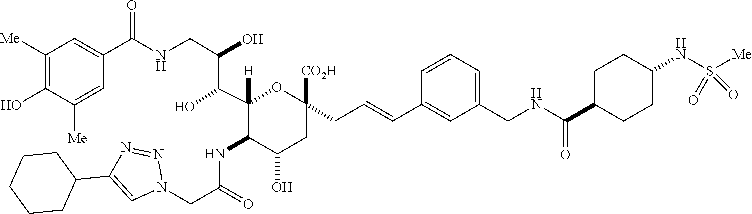 | H | 938.43 | 938.7 | 6.9 | 1.52 |
| A-282 | 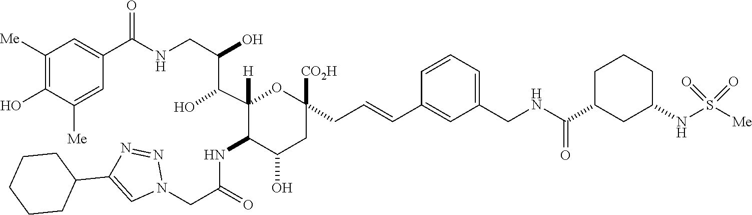 | H | 938.43 | 938.6 | 6.2 | 1.68 |
| A-283 | 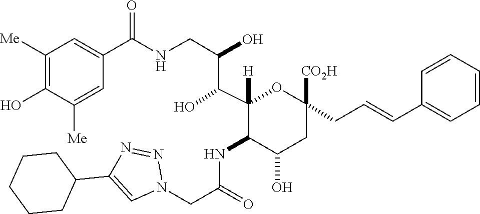 [SMILES: Cc1cc(C(=O)NC[C@@H](O)[C@@H](O)[C@@H]2O[C@@](C/C=C/c3ccccc3)(C(=O)O)C[C@H](O)[C@H]2NC(=O)Cn2cc(C3CCCCC3)nn2)cc(C)c1O] | H | 706.34 | 706.5 | 2.1 | 101.9 |
| A-284 | 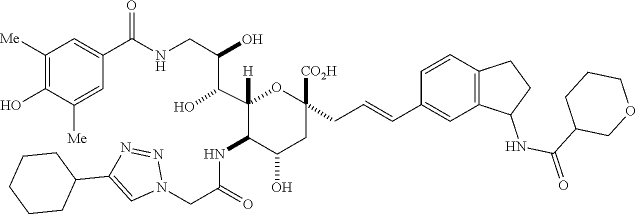 [SMILES: Cc1cc(C(=O)NC[C@@H](O)[C@@H](O)[C@@H]2O[C@@](C/C=C/c3ccc4c(c3)C(NC(=O)C3CCCOC3)CC4)(C(=O)O)C[C@H](O)[C@H]2NC(=O)Cn2cc(C3CCCCC3)nn2)cc(C)c1O] | H | 873.44 | 873.39 | 6.8 | 1.66 |
| A-285 | 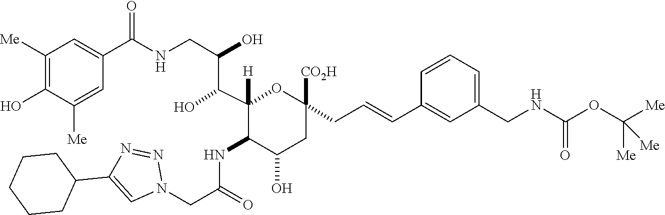 [SMILES: Cc1cc(C(=O)NC[C@@H](O)[C@@H](O)[C@@H]2O[C@@](C/C=C/c3cccc(CNC(=O)OC(C)(C)C)c3)(C(=O)O)C[C@H](O)[C@H]2NC(=O)Cn2cc(C3CCCCC3)nn2)cc(C)c1O] | H | 835.42 | 835.6 | 9.0 | 9.98 |
| A-286 | 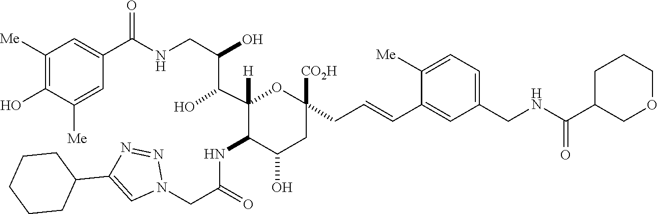 [SMILES: Cc1ccc(CNC(=O)C2CCCOC2)cc1/C=C/C[C@]1(C(=O)O)C[C@H](O)[C@@H](NC(=O)Cn2cc(C3CCCCC3)nn2)[C@H]([C@H](O)[C@H](O)CNC(=O)c2cc(C)c(O)c(C)c2)O1] | H | 861.44 | 861.6 | 2.2 | 2.84 |
| A-287 | 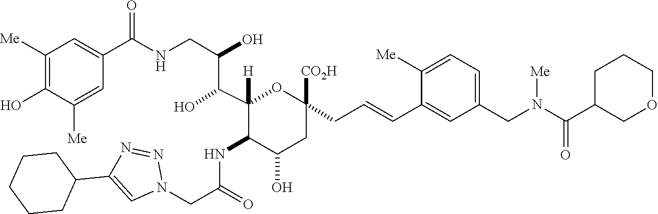 [SMILES: Cc1ccc(CN(C)C(=O)C2CCCOC2)cc1/C=C/C[C@]1(C(=O)O)C[C@H](O)[C@@H](NC(=O)Cn2cc(C3CCCCC3)nn2)[C@H]([C@H](O)[C@H](O)CNC(=O)c2cc(C)c(O)c(C)c2)O1] | H | 875.42 | 875.5 | 6.0 | 1.16 |
| A-288 | 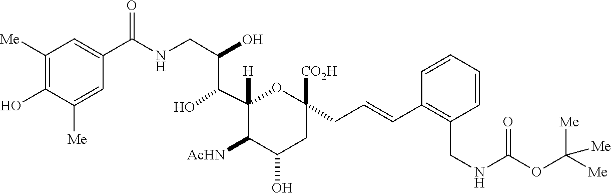 [SMILES: CC(=O)N[C@H]1[C@H]([C@H](O)[C@H](O)CNC(=O)c2cc(C)c(O)c(C)c2)O[C@@](C/C=C/c2ccccc2CNC(=O)OC(C)(C)C)(C(=O)O)C[C@@H]1O] | H | 686.32 | 686.31 | 5.3 | 2.68 |
| A-289 | 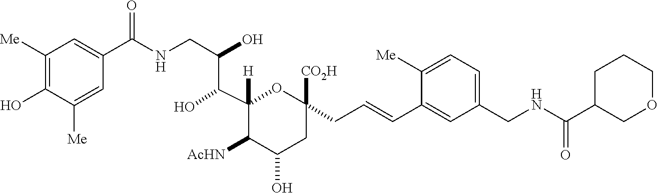 [SMILES: CC(=O)N[C@H]1[C@H]([C@H](O)[C@H](O)CNC(=O)c2cc(C)c(O)c(C)c2)O[C@@](C/C=C/c2cc(CNC(=O)C3CCCOC3)ccc2C)(C(=O)O)C[C@@H]1O] | H | 712.34 | 712.5 | 5.3 | 2.68 |
| A-290 | 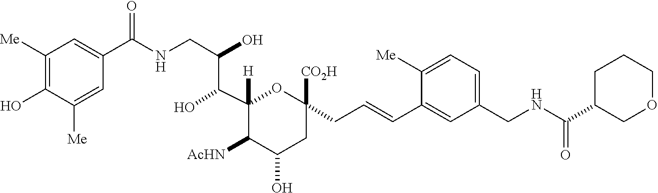 | H | 712.34 | 712.5 | 2.0 | 20.4 |
| A-291 | 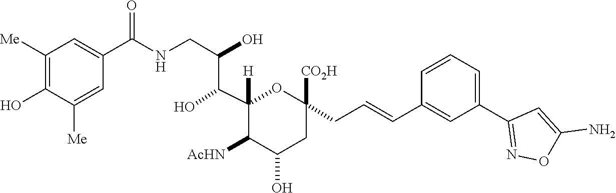 [SMILES: CC(=O)N[C@H]1[C@H]([C@H](O)[C@H](O)CNC(=O)c2cc(C)c(O)c(C)c2)O[C@@](C/C=C/c2cccc(-c3cc(N)on3)c2)(C(=O)O)C[C@@H]1O] | H | 639.26 | 639.4 | 2.9 | 4.30 |
| A-292 | 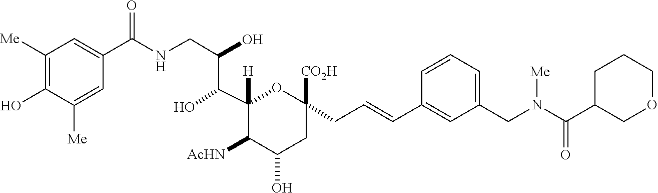 [SMILES: CC(=O)N[C@H]1[C@H]([C@H](O)[C@H](O)CNC(=O)c2cc(C)c(O)c(C)c2)O[C@@](C/C=C/c2cccc(CN(C)C(=O)C3CCCOC3)c2)(C(=O)O)C[C@@H]1O] | H | 712.34 | 712.39 | 4.0 | 3.02 |
| A-293 | 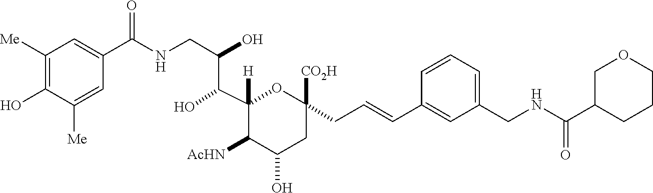 [SMILES: CC(=O)N[C@H]1[C@H]([C@H](O)[C@H](O)CNC(=O)c2cc(C)c(O)c(C)c2)O[C@@](C/C=C/c2cccc(CNC(=O)C3CCCOC3)c2)(C(=O)O)C[C@@H]1O] | H | 698.32 | 698.5 | 4.2 | 7.94 |
| A-294 | 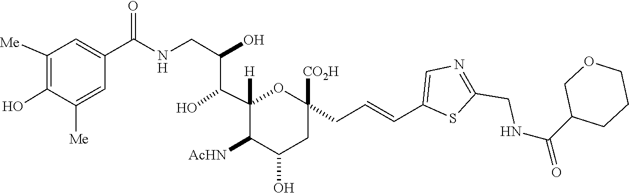 [SMILES: CC(=O)N[C@H]1[C@H]([C@H](O)[C@H](O)CNC(=O)c2cc(C)c(O)c(C)c2)O[C@@](C/C=C/c2cnc(CNC(=O)C3CCCOC3)s2)(C(=O)O)C[C@@H]1O] | H | 705.28 | 705.4 | 4.2 | 4.61 |
| A-295 | 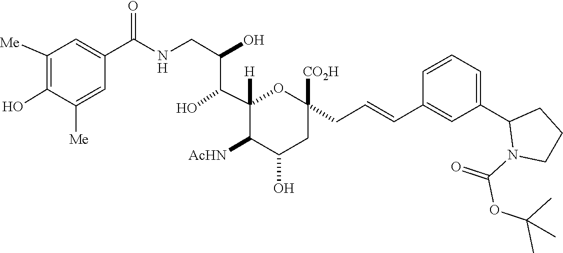 [SMILES: CC(=O)N[C@H]1[C@H]([C@H](O)[C@H](O)CNC(=O)c2cc(C)c(O)c(C)c2)O[C@@](C/C=C/c2cccc(C3CCCN3C(=O)OC(C)(C)C)c2)(C(=O)O)C[C@@H]1O] | H | 726.36 | 726.46 | 4.0 | 6.64 |
| A-296 | 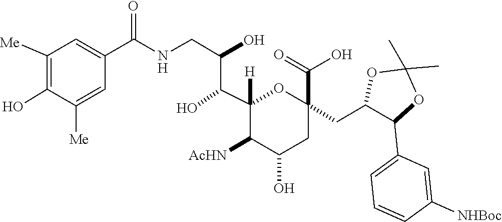 | H | 746.35 | 746.28 | 6.4 | 0.477 |
| A-297 | 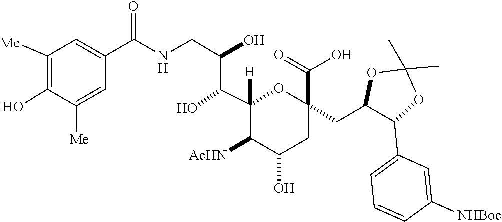 | H | 746.35 | 746.28 | | |
| A-298 | 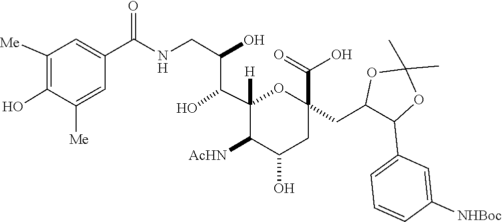 [SMILES: CC(=O)N[C@H]1[C@H]([C@H](O)[C@H](O)CNC(=O)c2cc(C)c(O)c(C)c2)O[C@@](CC2OC(C)(C)OC2c2cccc(NC(=O)OC(C)(C)C)c2)(C(=O)O)C[C@@H]1O] | H | 746.35 | 746.24 | | |
| A-299 | 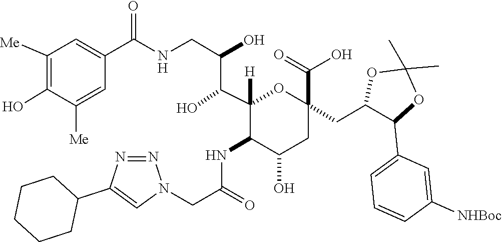 | Na | 917.44 | 917.35 | 8.0 | 0.512 |
| A-300 | 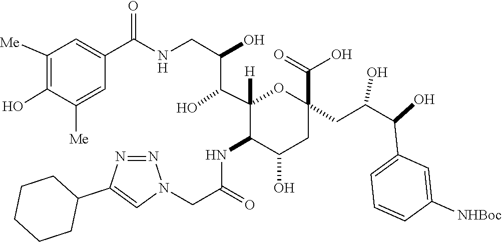 | H | 855.41 | 855.41 | 1.6 | 44.6 |
| A-301 | 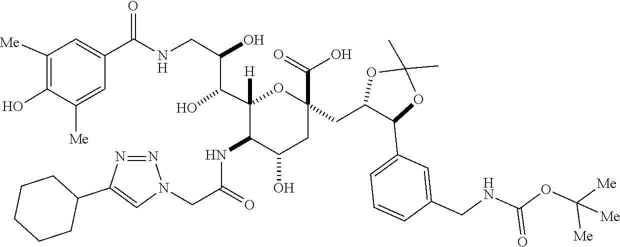 | Na | 931.45 | 931.32 | 3.5 | 5.26 |
| A-302 | 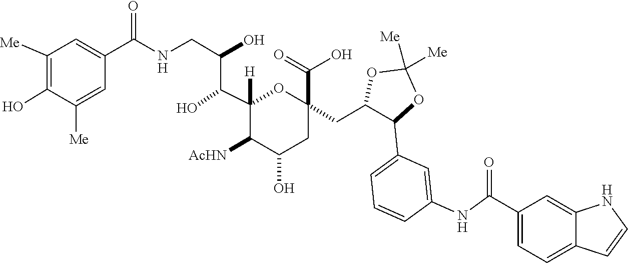 | H | 789.33 | 789.24 | 4.9 | 1.50 |
| A-303 | 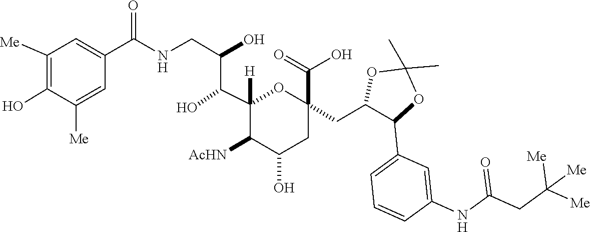 | Na | 766.36 | 766.39 | 4.9 | 1.50 |
| A-304 | 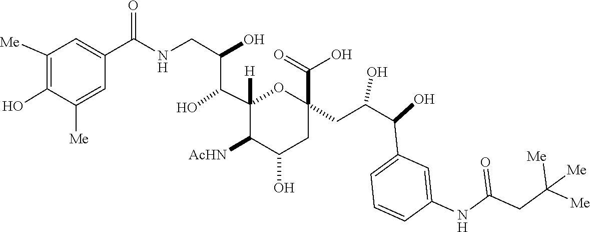 | Na | 726.36 | 726.40 | 3.9 | 4.70 |
| A-305 | 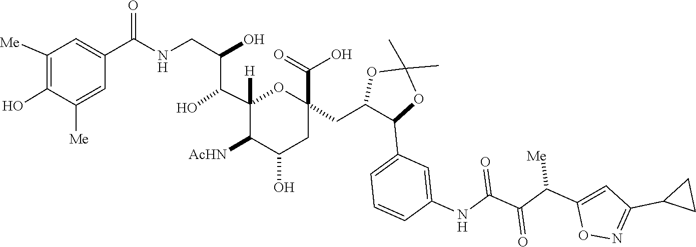 | Na | 845.37 | 845.29 | 5.4 | 0.792 |
| A-306 | 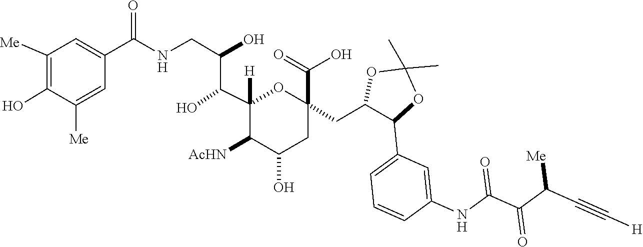 | H | 740.33 | 740.41 | | 5.62 |
| A-307 | 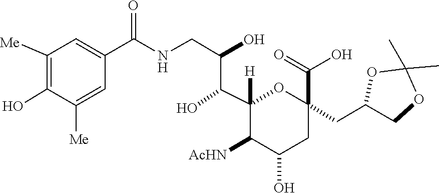 | H | 555.25 | 555.39 | 0.0 | 103 |
| A-308 | 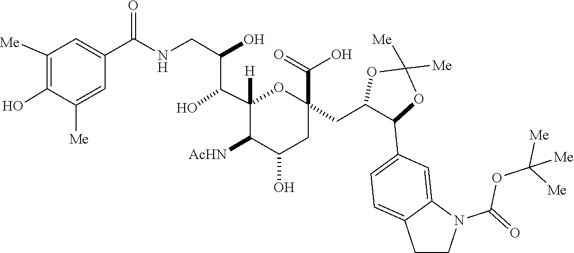 | H | 772.36 | 772.29 | | 2.94 |
| A-309 | 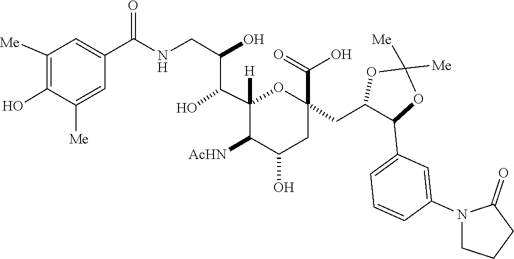 | H | 714.32 | 714.30 | 5.2 | 0.54 |
| A-310 | 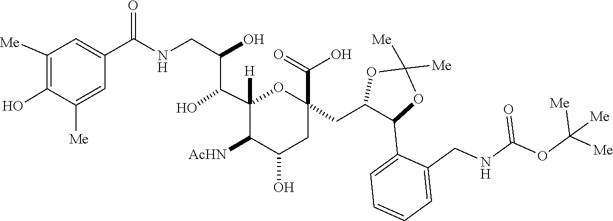 | H | 760.36 | 760.30 | 3.5 | 3.24 |
| A-311 | 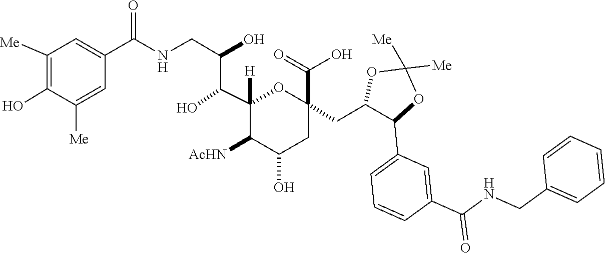 | Na | 786.33 | 786.34 | 0.5 | 6.36 |
| A-312 | 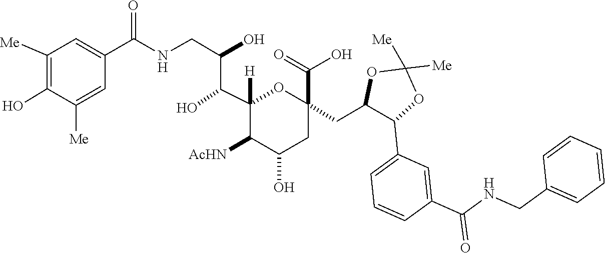 | Na | 786.33 | 786.30 | 4.5 | 0.159 |
| A-313 | 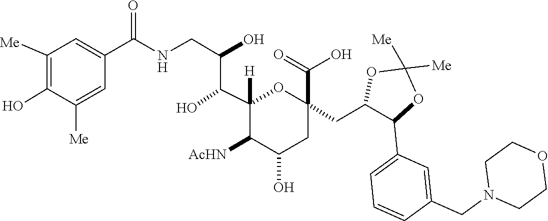 | H | 730.35 | 730.46 | 0.0 | 11.9 |
| A-314 | 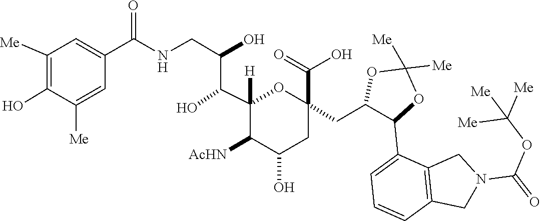 | H | 772.36 | 772.47 | 0.1 | 7.85 |
| A-315 | 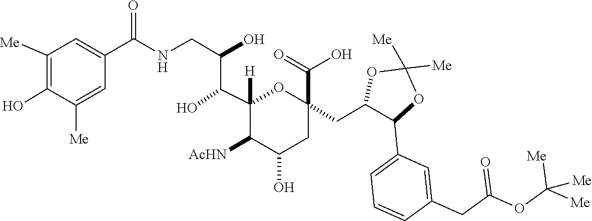 | H | 745.35 | 745.38 | 4.2 | 1.14 |
| A-316 | 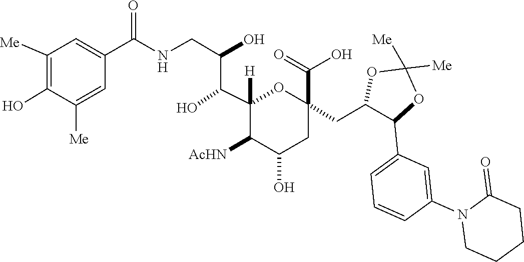 | H | 728.33 | 728.45 | 3.8 | 4.86 |
| A-317 | 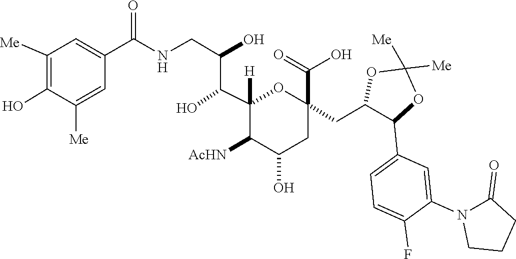 | H | 732.31 | 732.37 | 6.5 | 0.92 |
| A-318 | 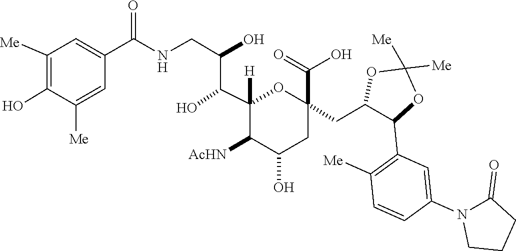 | H | 728.33 | 728.38 | 7.4 | 0.13 |
| A-319 | 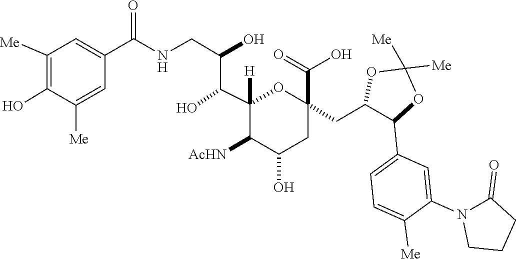 | H | 728.33 | 728.28 | 2.8 | 4.22 |
| A-320 | 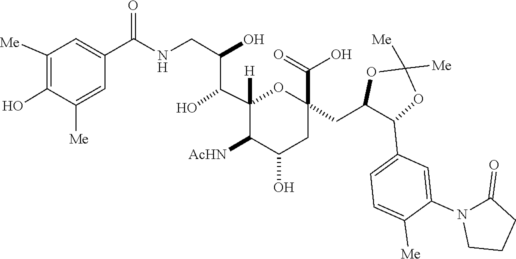 | Na | 750.33 | 750.31 | 4.8 | 0.96 |
| A-321 | 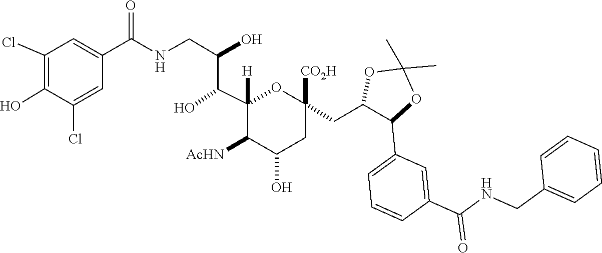 | H | 805.22 | 805.44 | 7.2 | 0.22 |
| A-322 | 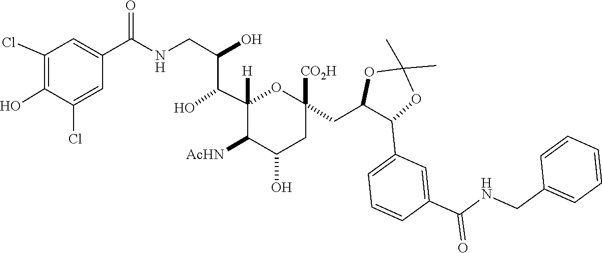 | H | 805.22 | 805.20 | 3.0 | 7.79 |
| A-323 | 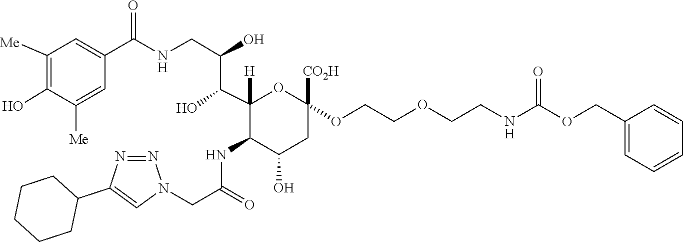 [SMILES: Cc1cc(C(=O)NC[C@@H](O)[C@@H](O)[C@@H]2O[C@@](OCCOCCNC(=O)OCc3ccccc3)(C(=O)O)C[C@H](O)[C@H]2NC(=O)Cn2cc(C3CCCCC3)nn2)cc(C)c1O] | H | 827.38 | 827.6 | 4.4 | 10.9 |
| A-324 | 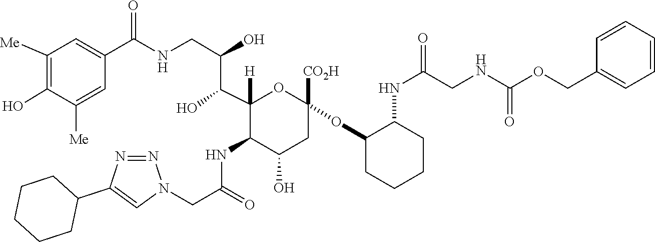 | H | 894.42 | 894.7 | 3.2 | 3.67 |
| A-325 | 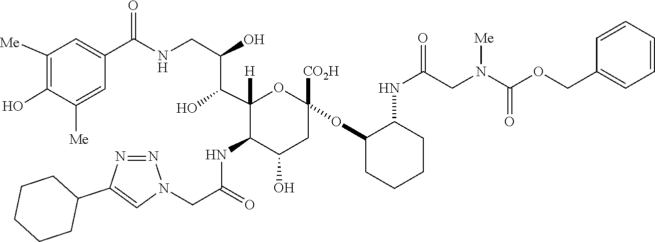 | H | 908.44 | 908.7 | 3.3 | 13.2 |
| A-326 | 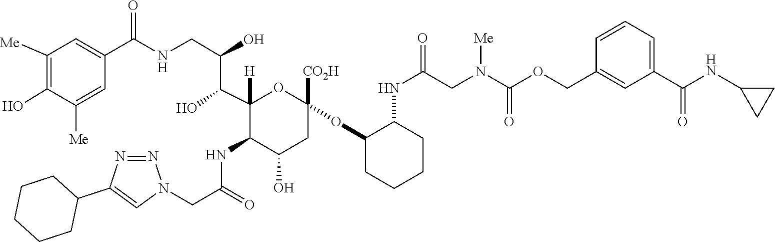 | H | 991.47 | 991.6 | 4.0 | 13.6 |
| A-327 | 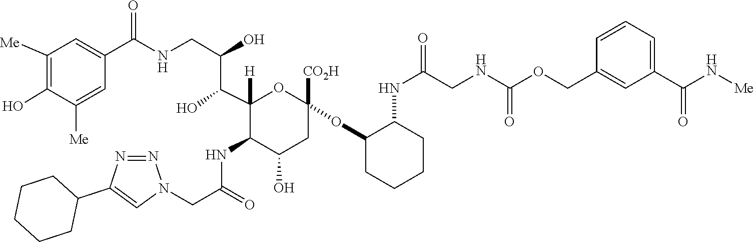 | H | 965.46 | 965.8 | 3.2 | 6.61 |
| A-328 | 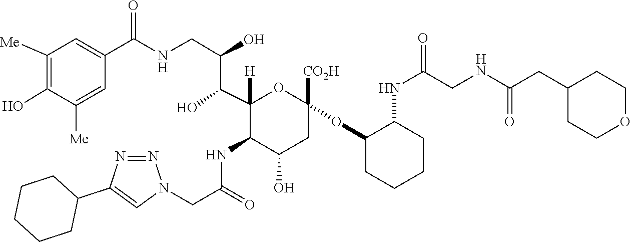 | H | 886.45 | 886.6 | 1.4 | 12.3 |
| A-329 | 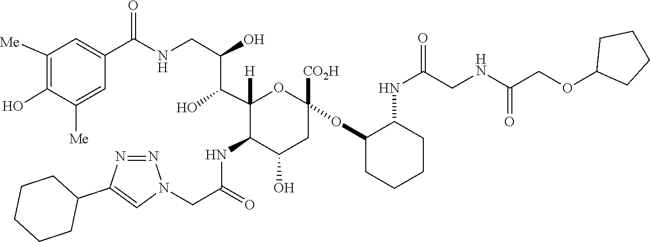 | H | 886.45 | 886.6 | 1.4 | 15.2 |
| A-330 | 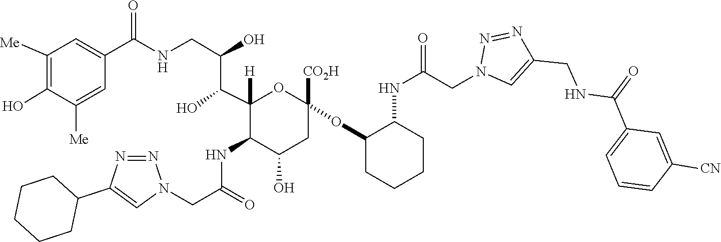 | H | 970.44 | 970.7 | 5.0 | 4.81 |
| A-331 | 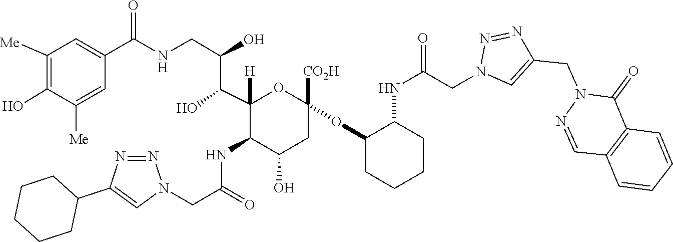 | H | 970.44 | 970.7 | 4.9 | 4.33 |
| A-332 | 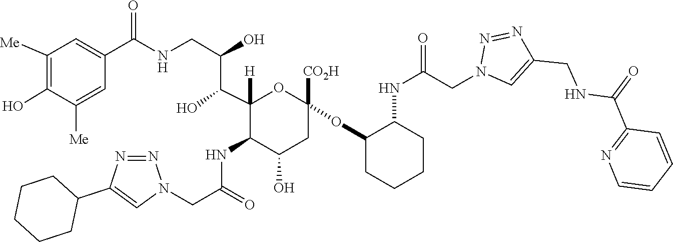 | H | 946.44 | 946.7 | 5.4 | 2.30 |
| A-333 | 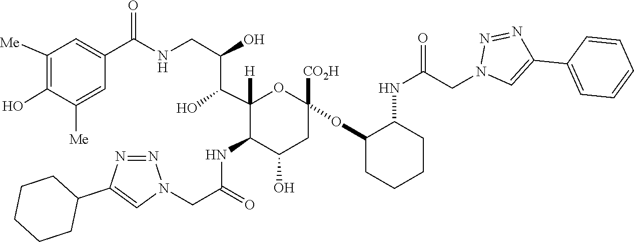 | H | 888.42 | 888.6 | 4.5 | 12.2 |
| A-334 | 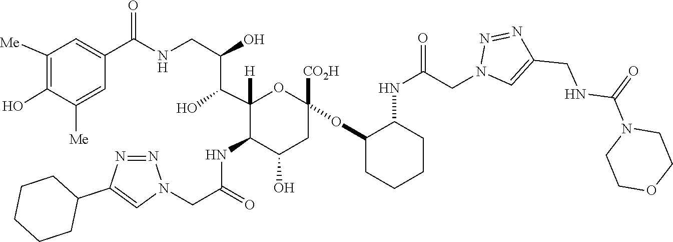 | H | 954.46 | 954.7 | 2.8 | 7.70 |
| A-335 | 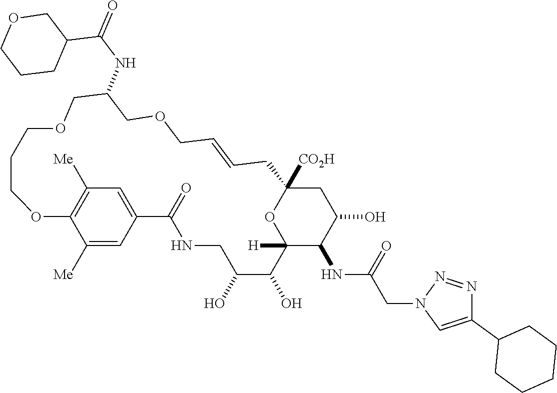 [SMILES: Cc1cc2cc(C)c1OCCCOC[C@H](NC(=O)C1CCCOC1)COC/C=C/C[C@]1(C(=O)O)C[C@H](O)[C@@H](NC(=O)Cn3cc(C4CCCCC4)nn3)[C@@H](O1)[C@H](O)[C@H](O)CNC2=O] | H | 885.43 | 885.45 | 6.4 | 0.601 |
| A-336 | 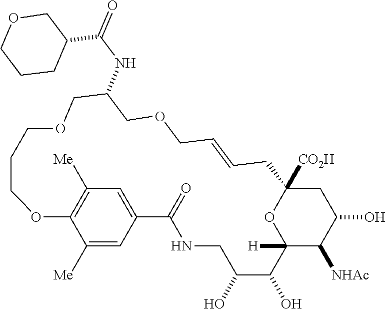 [SMILES: CC(=O)N[C@H]1[C@H]2O[C@](C(=O)O)(C/C=C/COC[C@@H](NC(=O)[C@@H]3CCCOC3)COCCCOc3c(C)cc(cc3C)C(=O)NC[C@@H](O)[C@H]2O)C[C@@H]1O] | H | 736.36 | 736.34 | 3.6 | 4.19 |
| A-337 | 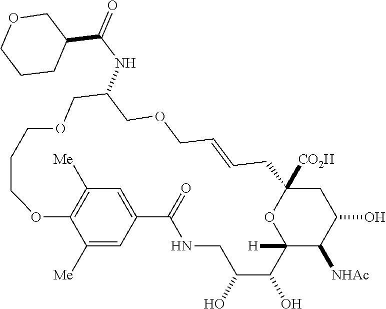 [SMILES: CC(=O)N[C@H]1[C@H]2O[C@](C(=O)O)(C/C=C/COC[C@@H](NC(=O)[C@H]3CCCOC3)COCCCOc3c(C)cc(cc3C)C(=O)NC[C@@H](O)[C@H]2O)C[C@@H]1O] | H | 736.36 | 736.31 | 2.2 | 14.13 |
| A-338 | 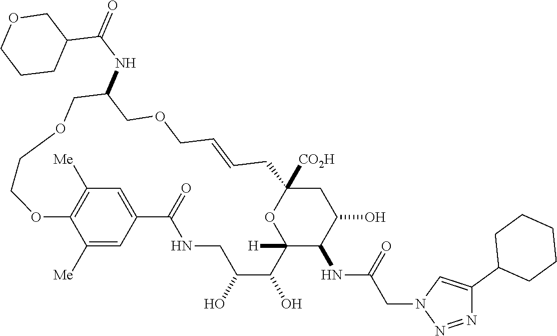 [SMILES: Cc1cc2cc(C)c1OCCOC[C@@H](NC(=O)C1CCCOC1)COC/C=C/C[C@]1(C(=O)O)C[C@H](O)[C@@H](NC(=O)Cn3cc(C4CCCCC4)nn3)[C@@H](O1)[C@H](O)[C@H](O)CNC2=O] | H | 871.44 | 871.40 | 5.6 | 3.82 |
| A-339 | 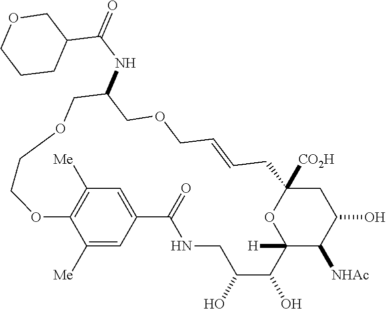 [SMILES: CC(=O)N[C@H]1[C@H]2O[C@](C(=O)O)(C/C=C/COC[C@H](NC(=O)C3CCCOC3)COCCOc3c(C)cc(cc3C)C(=O)NC[C@@H](O)[C@H]2O)C[C@@H]1O] | H | 722.35 | 722.28 | 4.4 | 5.91 |
| A-340 | 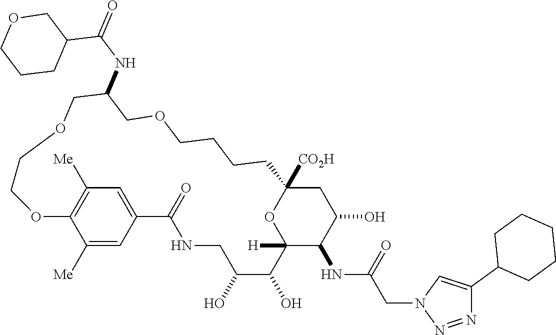 [SMILES: Cc1cc2cc(C)c1OCCOC[C@@H](NC(=O)C1CCCOC1)COCCCC[C@]1(C(=O)O)C[C@H](O)[C@@H](NC(=O)Cn3cc(C4CCCCC4)nn3)[C@@H](O1)[C@H](O)[C@H](O)CNC2=O] | H | 873.46 | 873.33 | 4.22 | 3.50 |
| A-341 | 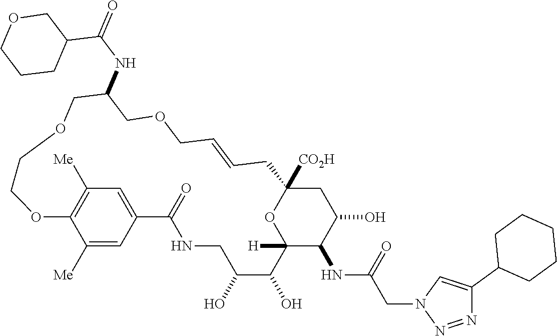 [SMILES: Cc1cc2cc(C)c1OCCOC[C@@H](NC(=O)C1CCCOC1)COC/C=C/C[C@]1(C(=O)O)C[C@H](O)[C@@H](NC(=O)Cn3cc(C4CCCCC4)nn3)[C@@H](O1)[C@H](O)[C@H](O)CNC2=O] | H | 871.44 | 871.37 | 2.7 | 9.98 |
| A-342 | 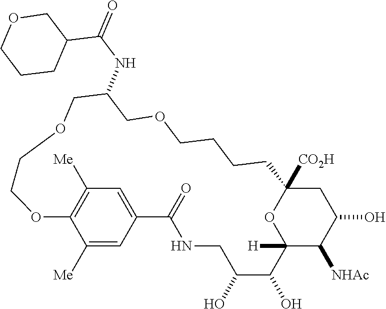 [SMILES: CC(=O)N[C@H]1[C@H]2O[C@](C(=O)O)(CCCCOC[C@@H](NC(=O)C3CCCOC3)COCCOc3c(C)cc(cc3C)C(=O)NC[C@@H](O)[C@H]2O)C[C@@H]1O] | H | 724.46 | 724.26 | 1.5 | 45.4 |
| A-343 | 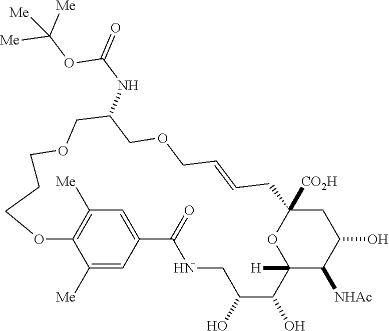 [SMILES: CC(=O)N[C@H]1[C@H]2O[C@](C(=O)O)(C/C=C/COC[C@@H](NC(=O)OC(C)(C)C)COCCCOc3c(C)cc(cc3C)C(=O)NC[C@@H](O)[C@H]2O)C[C@@H]1O] | H | 724.46 | 724.49 | 4.6 | 6.54 |
| A-344 | 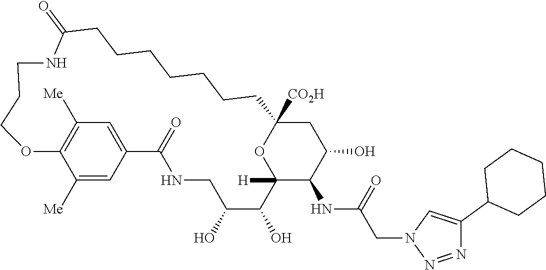 [SMILES: Cc1cc2cc(C)c1OCCCNC(=O)CCCCCCC[C@]1(C(=O)O)C[C@H](O)[C@@H](NC(=O)Cn3cc(C4CCCCC4)nn3)[C@@H](O1)[C@H](O)[C@H](O)CNC2=O] | H | 771.42 | 771.60 | 1.52 | 18.00 |
| A-345 | 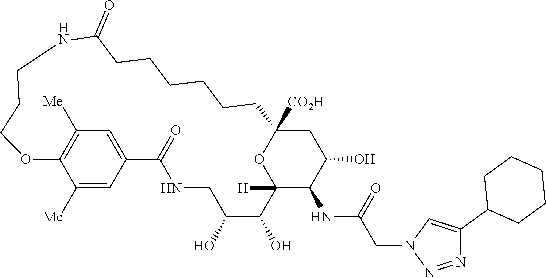 [SMILES: Cc1cc2cc(C)c1OCCCNC(=O)CCCCCC[C@]1(C(=O)O)C[C@H](O)[C@@H](NC(=O)Cn3cc(C4CCCCC4)nn3)[C@@H](O1)[C@H](O)[C@H](O)CNC2=O] | H | 757.41 | 756.41 | 3.2 | 21.37 |
| A-346 | 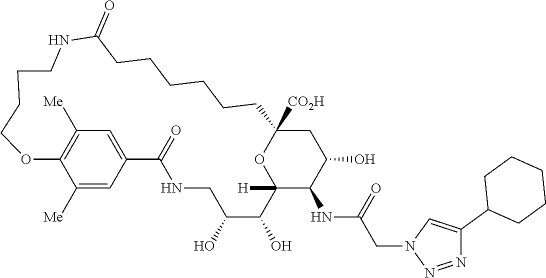 [SMILES: Cc1cc2cc(C)c1OCCCCNC(=O)CCCCCC[C@]1(C(=O)O)C[C@H](O)[C@@H](NC(=O)Cn3cc(C4CCCCC4)nn3)[C@@H](O1)[C@H](O)[C@H](O)CNC2=O] | H | 771.42 | 771.76 | 3.4 | 13.31 |
| A-347 | 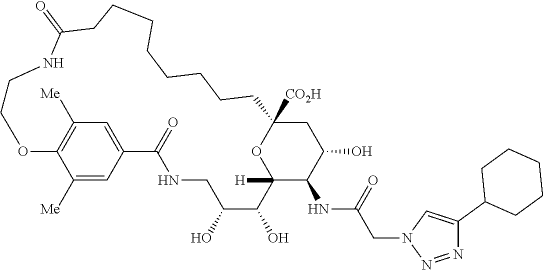 [SMILES: Cc1cc2cc(C)c1OCCNC(=O)CCCCCCCC[C@]1(C(=O)O)C[C@H](O)[C@@H](NC(=O)Cn3cc(C4CCCCC4)nn3)[C@@H](O1)[C@H](O)[C@H](O)CNC2=O] | H | 771.42 | 771.44 | 3.6 | 18.97 |
| A-348 | 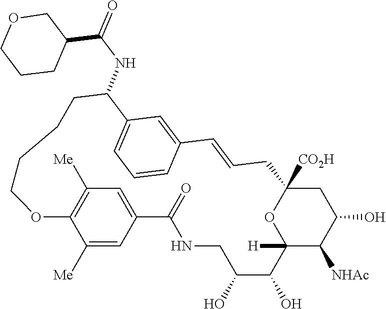 [SMILES: CC(=O)N[C@H]1[C@H]2O[C@](C(=O)O)(C/C=C/c3cccc(c3)[C@@H](NC(=O)[C@H]3CCCOC3)CCCCOc3c(C)cc(cc3C)C(=O)NC[C@@H](O)[C@H]2O)C[C@@H]1O] | H | 752.37 | | 3.6 | 10.10 |
| A-349 | 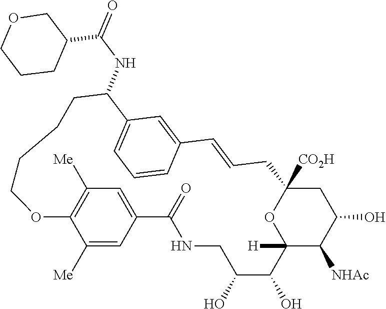 [SMILES: CC(=O)N[C@H]1[C@H]2O[C@](C(=O)O)(C/C=C/c3cccc(c3)[C@@H](NC(=O)[C@@H]3CCCOC3)CCCCOc3c(C)cc(cc3C)C(=O)NC[C@@H](O)[C@H]2O)C[C@@H]1O] | H | 752.37 | | 5.5 | 5.24 |
| A-350 | 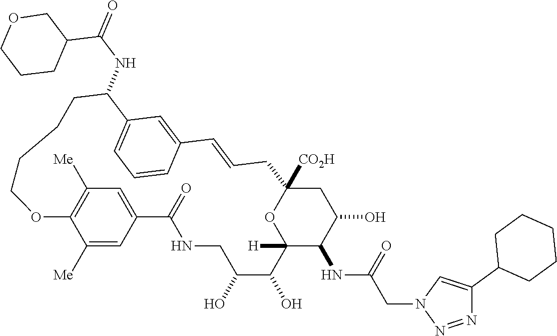 [SMILES: Cc1cc2cc(C)c1OCCCC[C@H](NC(=O)C1CCCOC1)c1cccc(c1)/C=C/C[C@]1(C(=O)O)C[C@H](O)[C@@H](NC(=O)Cn3cc(C4CCCCC4)nn3)[C@@H](O1)[C@H](O)[C@H](O)CNC2=O] | Y | 901.47 | 901.40 | | 13.27 |
| A-351 | 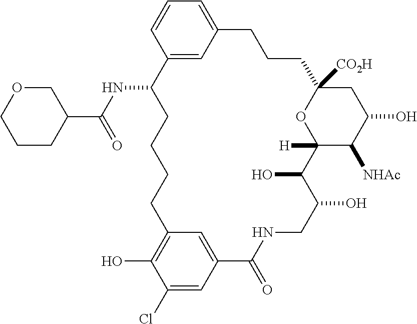 [SMILES: CC(=O)N[C@H]1[C@H]2O[C@](C(=O)O)(CCCc3cccc(c3)[C@@H](NC(=O)C3CCCOC3)CCCCc3cc(cc(Cl)c3O)C(=O)NC[C@@H](O)[C@H]2O)C[C@@H]1O] | H | 760.32 | 760.27 | 3.1 | 14.99 |
| A-352 | 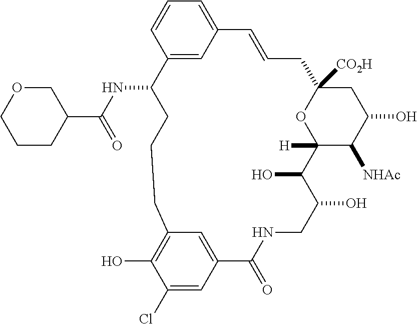 [SMILES: CC(=O)N[C@H]1[C@H]2O[C@](C(=O)O)(C/C=C/c3cccc(c3)[C@@H](NC(=O)C3CCCOC3)CCCc3cc(cc(Cl)c3O)C(=O)NC[C@@H](O)[C@H]2O)C[C@@H]1O] | H | 744.29 | 744.16 | 0.7 | 94.0 |
| A-353 | 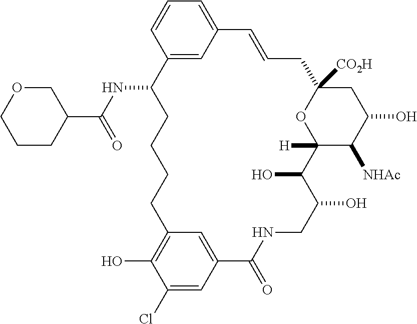 [SMILES: CC(=O)N[C@H]1[C@H]2O[C@](C(=O)O)(C/C=C/c3cccc(c3)[C@@H](NC(=O)C3CCCOC3)CCCCc3cc(cc(Cl)c3O)C(=O)NC[C@@H](O)[C@H]2O)C[C@@H]1O] | H | 758.30 | 758.27 | 3.6 | 18.97 |
| A-354 | 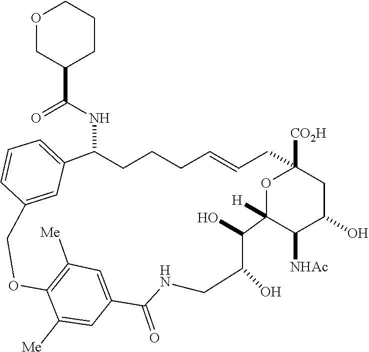 [SMILES: CC(=O)N[C@H]1[C@H]2O[C@](C(=O)O)(C/C=C/CCC[C@@H](NC(=O)[C@@H]3CCCOC3)c3cccc(c3)COc3c(C)cc(cc3C)C(=O)NC[C@@H](O)[C@H]2O)C[C@@H]1O] | H | 752.37 | 752.34 | 1.5 | 27.5 |
| A-355 | 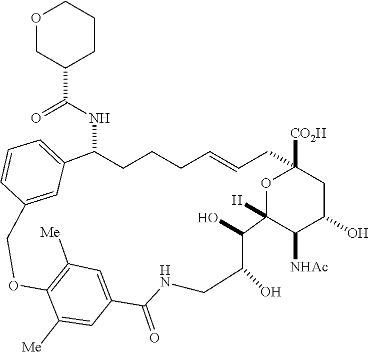 [SMILES: CC(=O)N[C@H]1[C@H]2O[C@](C(=O)O)(C/C=C/CCC[C@@H](NC(=O)[C@H]3CCCOC3)c3cccc(c3)COc3c(C)cc(cc3C)C(=O)NC[C@@H](O)[C@H]2O)C[C@@H]1O] | H | 752.37 | 752.36 | 1.3 | 35.4 |
| A-356 | 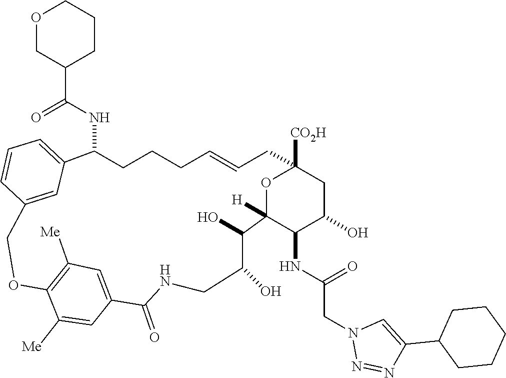 [SMILES: Cc1cc2cc(C)c1OCc1cccc(c1)[C@H](NC(=O)C1CCCOC1)CCC/C=C/C[C@]1(C(=O)O)C[C@H](O)[C@@H](NC(=O)Cn3cc(C4CCCCC4)nn3)[C@@H](O1)[C@H](O)[C@H](O)CNC2=O] | H | 901.47 | 901.51 | 4.8 | 1.51 |
| A-357 | 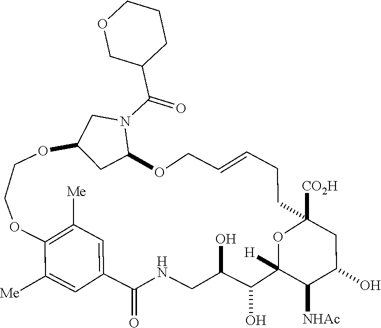 [SMILES: CC(=O)N[C@H]1[C@H]2O[C@](C(=O)O)(CC/C=C/CO[C@H]3C[C@H](CN3C(=O)C3CCCOC3)OCCOc3c(C)cc(cc3C)C(=O)NC[C@@H](O)[C@H]2O)C[C@@H]1O] | H | 748.36 | 748.33 | 1.1 | 66.1 |
| A-358 | 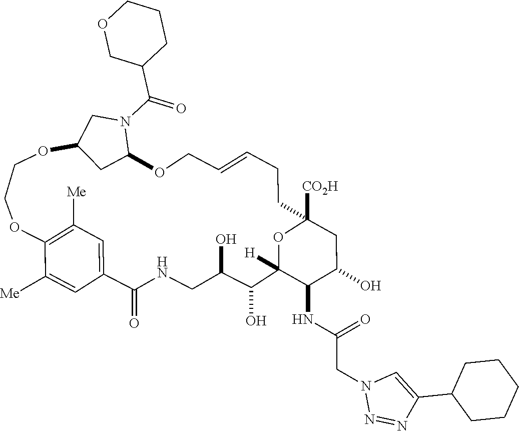 [SMILES: Cc1cc2cc(C)c1OCCO[C@@H]1C[C@H](OC/C=C/CC[C@]3(C(=O)O)C[C@H](O)[C@@H](NC(=O)Cn4cc(C5CCCCC5)nn4)[C@@H](O3)[C@H](O)[C@H](O)CNC2=O)N(C(=O)C2CCCOC2)C1] | H | 897.46 | 897.46 | 3.5 | 9.43 |
| A-359 | 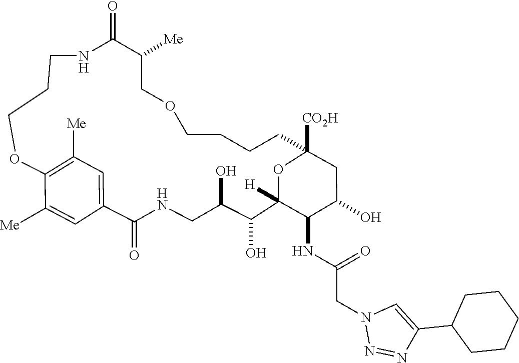 [SMILES: Cc1cc2cc(C)c1OCCCNC(=O)[C@H](C)COCCCC[C@]1(C(=O)O)C[C@H](O)[C@@H](NC(=O)Cn3cc(C4CCCCC4)nn3)[C@@H](O1)[C@H](O)[C@H](O)CNC2=O] | H | 787.42 | 787.52 | 1.1 | 59.8 |
| A-360 | 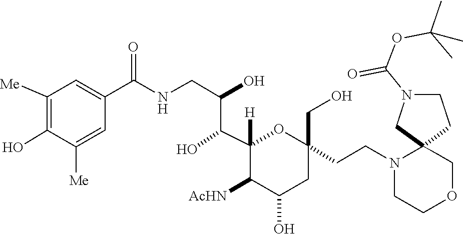 | H | 695.38 | 695.6 | −0.3 | >100 |
| A-361 A-002 | 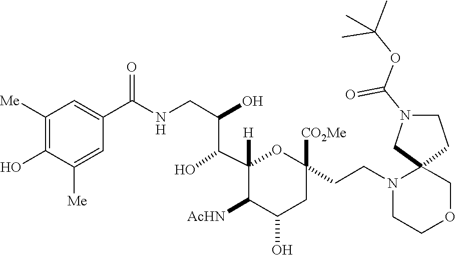 | H | 723.37 | 723.54 | −0.3 | 49.0 |
| A-363 | 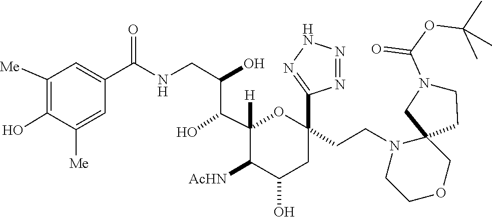 | H | 733.38 | 733.55 | 2.6 | 6.36 |
| A-362 | 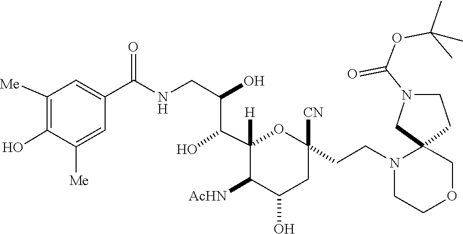 | H | 690.37 | 690.6 | −0.3 | >100 |
| A-364 | 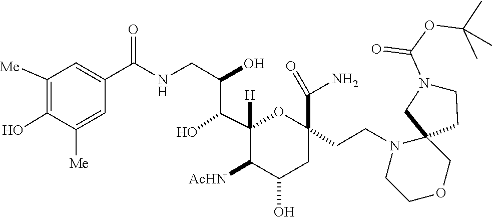 | H | 708.38 | 708.6 | 0.1 | >100 |
| A-365 | 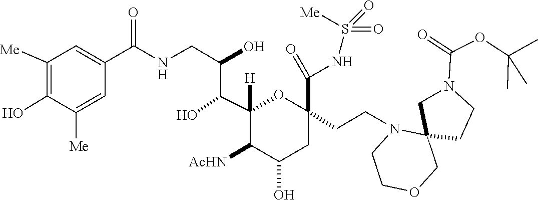 | H | 786.36 | 786.65 | −0.3 | 37.5 |
| A-366 | 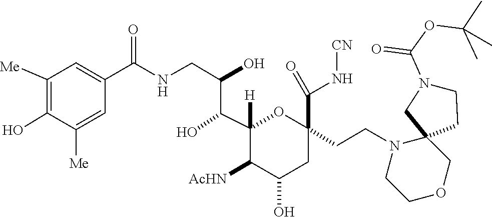 | H | 733.37 | 733.63 | 2.9 | 2.83 |
| A-367 | 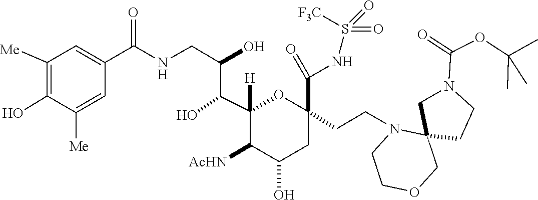 | H | 840.33 | 840.63 | 0.1 | >100 |
| A-368 | 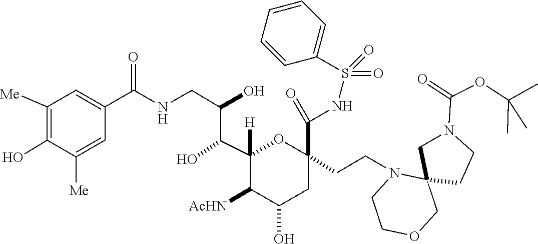 | H | 848.37 | 848.68 | −0.1 | 14.96 |
| A-369 | See structure above | H | 871.45 | 871.72 | | |
| A-370 | See structure above | H | 995.09 | 994.93 | | |
| A-371 | See structure above | H | 1008.1 | 1007.8 | | |
| A-372 | See structure above | H | | | | |
| A-373 | See structure above | H | | | | |
| A-374 | See structure above | H | | Y | | |
| A-375 | See structure above | H | | | | |

### Liposomal Formulations for Lipid Conjugate Analogs

All formulations were prepared using the methodology as that described by S. Daun, et al. (J Clin Invest. 2019, 129(3), 1387-1401) and Bhattacherjee A. et al., J Controlled Release 2021; 338:680-693. The fluorescently labeled lipids, pHrodo-PEG-DSPE and AF647-PEG-DSPE are also described in those references. Commercially available lipids such as DSPC, Cholesterol, and DSPE-PEG were purchased and 10, 5, and 4 mg/mL stock solutions were made respectively. For naked liposomes 57, 38, 4.8, 0.1, and 0.1 mol % of DSPC, Cholesterol, DSPE-PEG, pHrodo-PEG-DSPE, AF647-PEG-DSPE were used respectively.

liposomes, 0.1% AF647PEG-DSPE was added to the lipid mixture.

Formulation A is composed of a 57:38:5 molar ratio of commercially available distearoyl phosphatidylcholine (DSPC), cholesterol, and polyethylene glycol-distearoyl phosphoethanolamine (PEG2K-DSPE). DSPC (2.25 mg, 0.00285 mmol), cholesterol (0.73 mg, 0.0019 mmol), PEG2K-DSPE (0.70 mg, 0.00025 mmol), 0.1% AF647PEG-DSPE, and 0.1% pHrodo-PEG-DSPE were added to the lipid mixture and dissolved in chloroform (0.5 mL). The mixture was sonicated for 5 min, after which time it was concentrated to dryness. The residue was dissolved in DMSO (0.2 mL) followed by lyophilization to obtain a white powder. The resulting solid was dissolved in standard PBS buffer (1 mL) and the resulting mixture was sonicated (5×30 s). The suspension was sequentially extruded though filters of 800-nm, 200-nm, and 100-nm controlled pore membranes (20 times per membrane) at room temperature using the dual-syringe method described by the membrane manufacturer (Nuclepore, Sigma-Aldrich). The final formulation was diluted with PBS to obtain the desired concentrations for biological evaluation.

Formulation B is composed of a 57:38:2:3 molar ratio of commercially available distearoyl phosphatidylcholine (DSPC), cholesterol, polyethylene glycol-distearoyl phosphoethanolamine (PEG2K-DSPE), and CD33L-PEG-DSPE (LP-CD33L) as described in Bhattacherjee A. et al., J Controlled Release 2021; 338:680-693. Formulation B was prepared in a similar fashion to formulation A with the exception of reducing the amount of PEG2K-DSPE to 2% (0.28 mg, 0.0001 mmol) and adding 3% CD33L-PEG-DSPE (preparation provided in S. Daun, et al. (J Clin Invest. 2019, 129(3), 1387-1401)).), 0.1% AF647PEG-DSPE, and 0.1% pHrodo-PEG-DSPE were also added to the lipid mixture.

Formulation C is composed of a 57:38:2:3 molar ratio of commercially available distearoyl phosphatidylcholine (DSPC), cholesterol, polyethylene glycol-distearoyl phosphoethanolamine (PEG2K-DSPE), and A-373. Formulation C was prepared in a similar fashion to formulation B with the exception of the replacement 3% CD33L-PEG-DSPE with 3% A-373 (0.57 mg, 0.00015 mmol). 0.1% AF647PEG-DSPE, and 0.1% pHrodo-PEG-DSPE were also added to the lipid mixture.

Formulation D is composed of a 57:38:2:3 molar ratio of commercially available distearoyl phosphatidylcholine (DSPC), cholesterol, polyethylene glycol-distearoyl phosphoethanolamine (PEG2K-DSPE), and A-374. Formulation D was prepared in a similar fashion to formulation B with the exception of the replacement 3% CD33L-PEG-DSPE with 3% A-374 (0.58 mg, 0.00015 mmol). 0.1% AF647PEG-DSPE, and 0.1% pHrodo-PEG-DSPE were also added to the lipid mixture.

Biological Results from Liposomal Formulations

### Method

Each formulation of liposome was labeled with pHrodo red and AF647, for the liposome internalization and cell surface binding, respectively. Internalization of the liposomes was assessed using the fluorescent signal from pHrodo incorporated into the liposomes and comparing it to the fluorescent signal from AF647 that represents total liposome binding. 1×10⁵ cells were pretreated with either 100 or 10 μM liposome formulation for 1 hour at 37° C. The liposome solutions were washed away, and the cells were incubated with polystyrene beads labeled with Pacific Blue for 30 minutes at 37° C. After incubation, the cells were washed with flow buffer, and resuspended in flow buffer, and subjected to flow cytometry as described in Bhattacherjee A. et al., J Controlled Release 2021; 338:680-693.

### Results

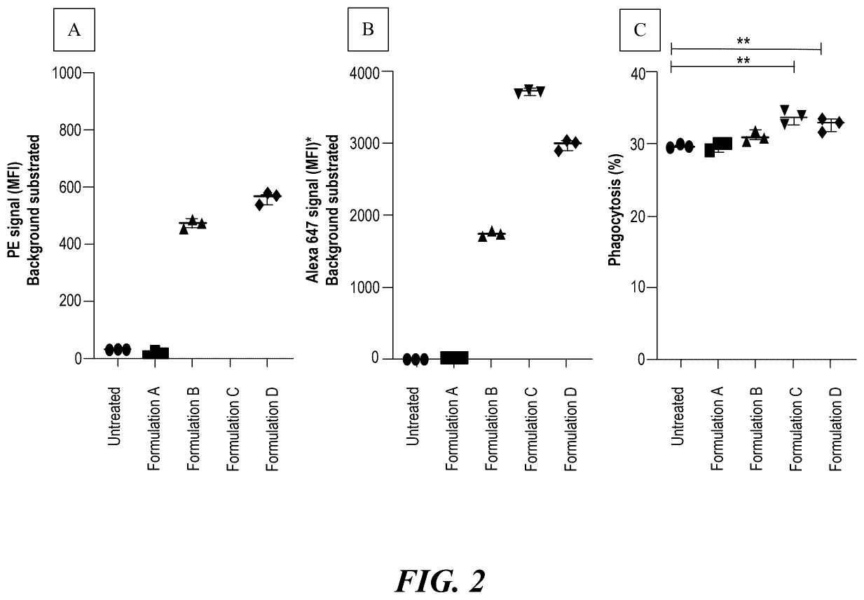

Formulation A, which is a naked liposome with no ligand for CD33, induced no liposome internalization or binding to cell surface. Formulations B, C, and D all induced liposome internalization and binding to cell surface at 10 and 100 μM (FIGS. 2, 3, 4). The effects were more robust at 100 μM than at 10 μM. Formulations C and D were more effective than Formulation B. Formulations C and D also enhanced phagocytosis of polystyrene beads at 100 μM. These results suggest that liposome-CD33 internalization through activation of the CD33 receptor leads to phagocytosis of particles confirming the results reported in the literature.

### Captions for FIGS. 1-5

FIG. 1. DSF Graph. Tm of CD33 protein alone (Gray lines) had an average value of approximate 55° C. Graph A: Addition of PBS control at 50 μM. Graph B: Addition of 50 μM of A-001 (Blue lines) to the CD33 protein induced significant Tm increases (ΔTm D) of 12.2±0.7° C. The increase in dTm D indicates that A-001 binds to and stabilizes the His-CD33 protein.

FIG. 2. Effect of 100 μM lipid formulations on internalization (A), binding (B) and phagocytosis (C) in microglia isolated from hCD33 mice. (Experiment 1) **p<0.01, student t test

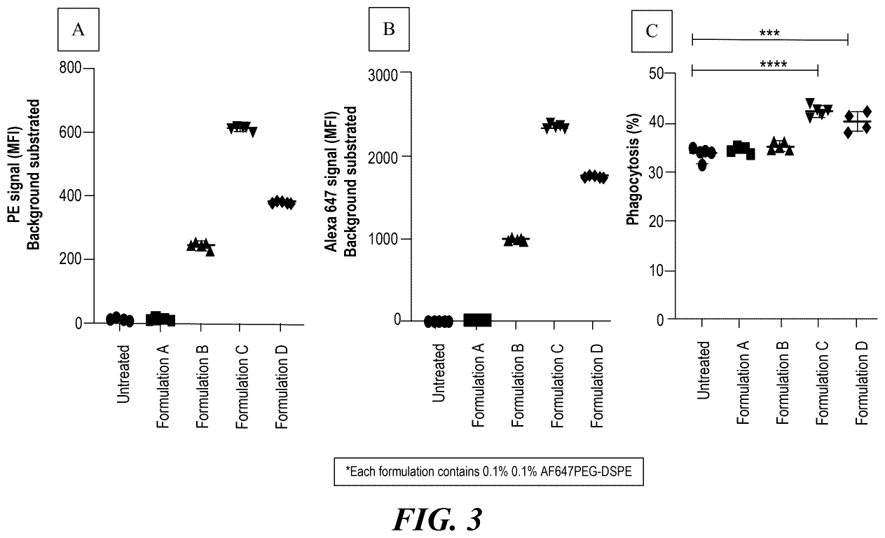

FIG. 3. Effect of 100 μM lipid formulations on internalization (A), binding (B) and phagocytosis (C) in microglia isolated from hCD33 mice. (Repeat of Experiment 1) ***p<0.001, ****p<0.0001, student t test

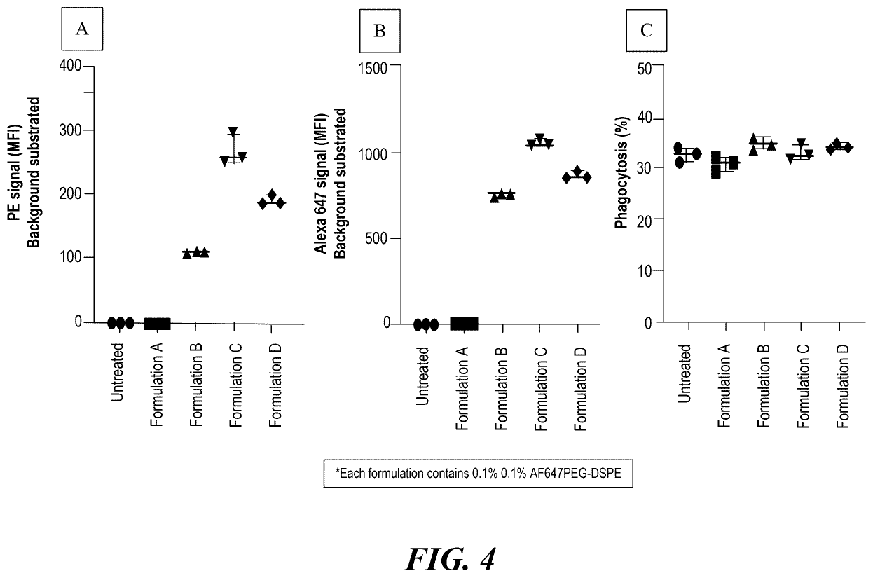

FIG. 4: Effect of 10 μM lipid formulations on internalization (A), binding (B) and phagocytosis (C) in microglia isolated from hCD33 mice.

1. A compound of Formula (I): 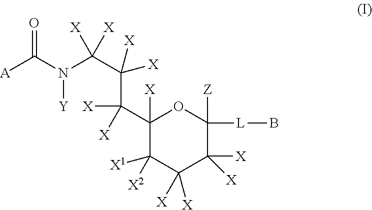
a tautomer thereof, a deuterated derivative of a compound of Formula (I), a deuterated derivative of a tautomer of a compound of Formula (I), or a pharmaceutically acceptable salt of any of the foregoing, wherein:
(viii) A is chosen from alkenyl groups, cycloalkyl groups, heterocyclic groups, aryl groups, and heteroaryl groups;
(ix) B is chosen from hydrogen, 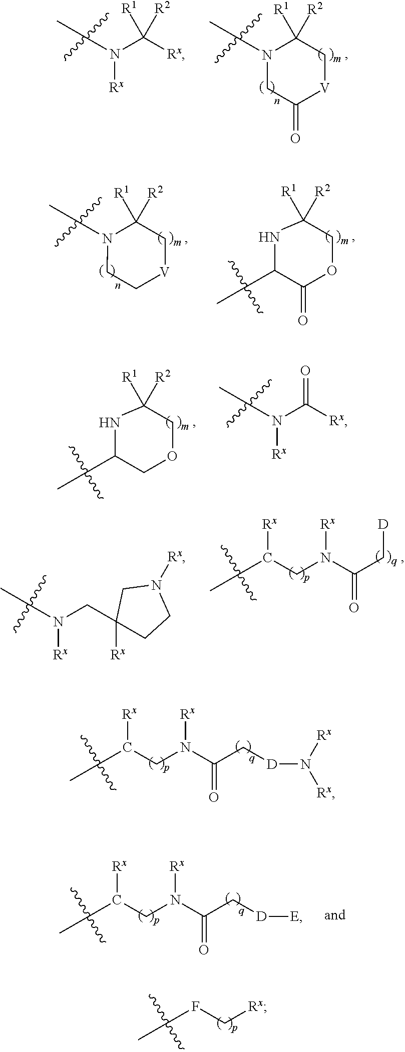
wherein
V is chosen from O, CH₂ and NR′; wherein R′ is chosen from hydrogen, hydroxy groups, linear alkyl groups, branched alkyl groups, and cyclic alkyl groups;
R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear, branched, cyclic alkoxy groups, cycloalkyl groups, heterocycloalkyl groups, aryl groups, and heteroaryl groups;
(x) L is chosen from C₁₋₁₀ linear alkylene groups, C₁₋₁₀ branched alkylene groups, C₁₋₁₀ cyclic alkylene groups, —C(O)—C₁₋₁₀ linear alkylene groups, C₁₋₁₀ branched alkylene groups, C₁₋₁₀ cyclic alkylene groups, C₁₋₁₀ linear alkylene-C(O)— groups, C₁₋₁₀ branched alkylene-C(O)-groups, C₁₋₁₀ cyclic alkylene-C(O)— groups, C₁₋₁₀ linear alkenylene groups, C₁₋₁₀ branched alkenylene groups, and C₁₋₁₀ cyclic alkenylene groups, 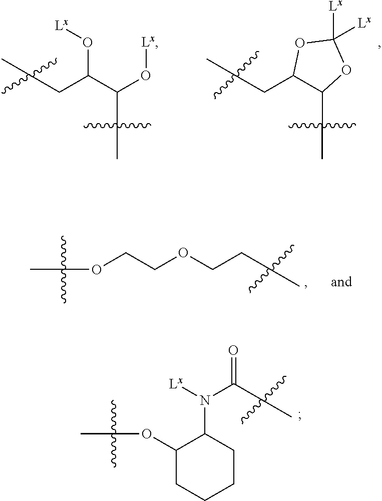
wherein each L^{x} is independently chosen from hydrogen, linear alkyl groups, branched alkyl groups, and cyclic alkyl groups;
(xi) each X is independently chosen from hydrogen, hydroxy groups, amino groups, linear alkyl groups, branched alkyl groups, and cyclic alkyl groups;
(xii) X¹ and X² are each independently chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(xiii) Y is chosen from hydrogen, linear alkyl groups, branched alkyl groups, and cyclic alkyl groups;
(xiv) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC₃-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

2. A compound of Formula (Ia): 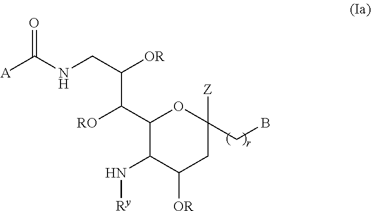
a tautomer thereof, a deuterated derivative of a compound of Formula (Ia), a deuterated derivative of a tautomer of a compound of Formula (Ia), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen, 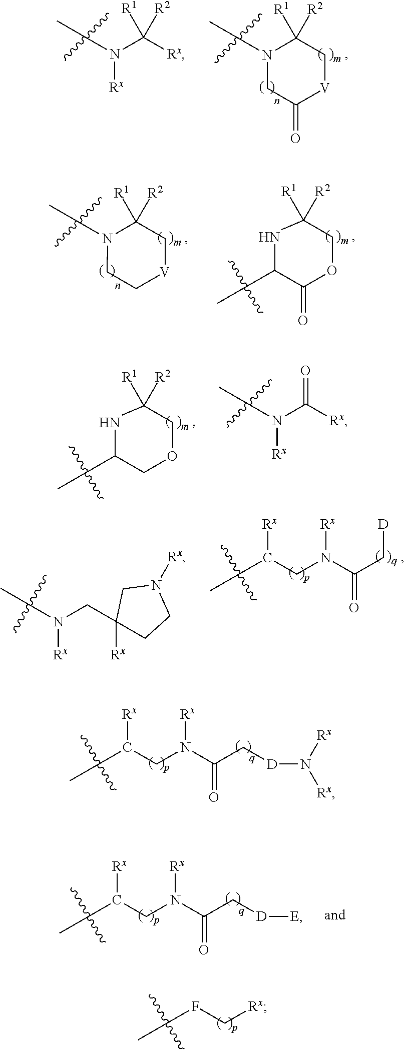
wherein
V is chosen from O, and NR;
R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear, branched, and cyclic alkyl groups;
(iv) R^{y} chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

3. A compound of Formula (Ib): 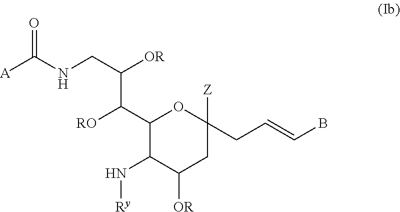
a tautomer thereof, a deuterated derivative of a compound of Formula (Ib), a deuterated derivative of a tautomer of a compound of Formula (Ib), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen, 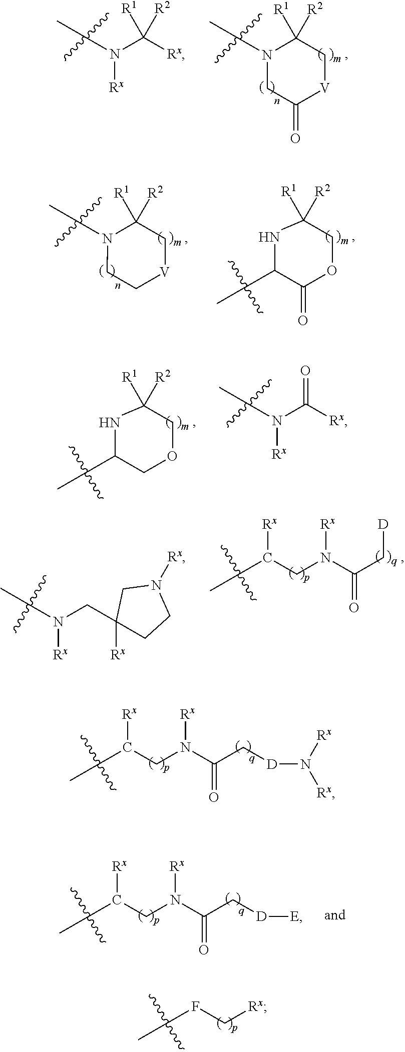
wherein
V is chosen from O, and NR;
R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear, branched, and cyclic alkyl groups;
(iv) R^{y} chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z},
(vi) —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

4. A compound of Formula (Ic): 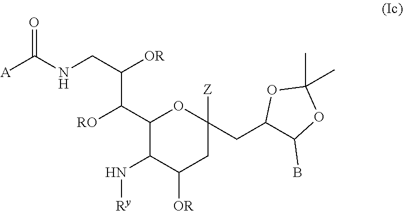
a tautomer thereof, a deuterated derivative of a compound of Formula (Ic), a deuterated derivative of a tautomer of a compound of Formula (Ic), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen, 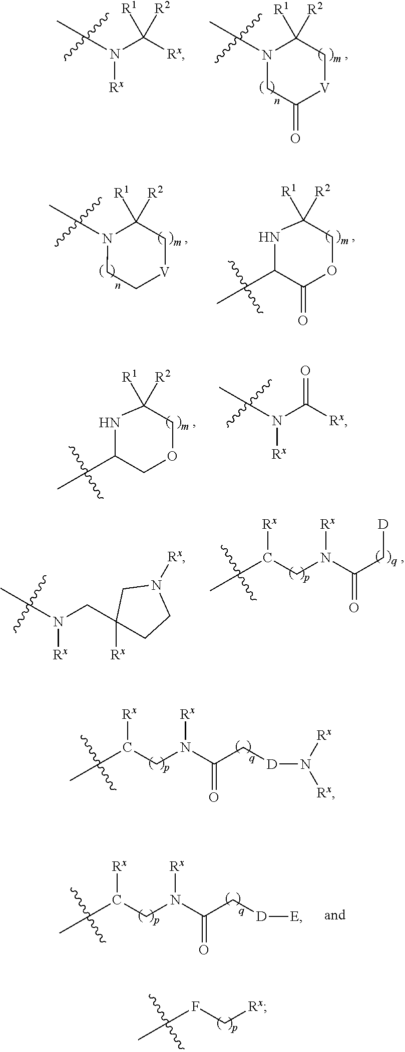
wherein
V is chosen from O, and NR;
R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear, branched, and cyclic alkyl groups;
(iv) R^{y} chosen from hydrogen, hydroxy groups, amino groups, linear alkyl groups, branched alkyl groups, and cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

5. A compound of Formula (Id): 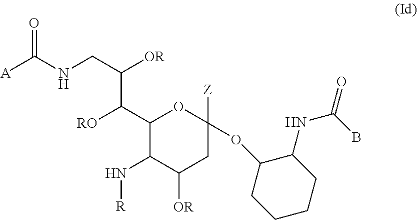
a tautomer thereof, a deuterated derivative of a compound of Formula (Id), a deuterated derivative of a tautomer of a compound of Formula (Id), or a pharmaceutically acceptable salt of any of the foregoing, wherein:
(i) A is chosen from alkenyl groups, cycloalkyl groups, heterocyclic groups, aryl groups, and heteroaryl groups;
(ii) B is chosen from hydrogen 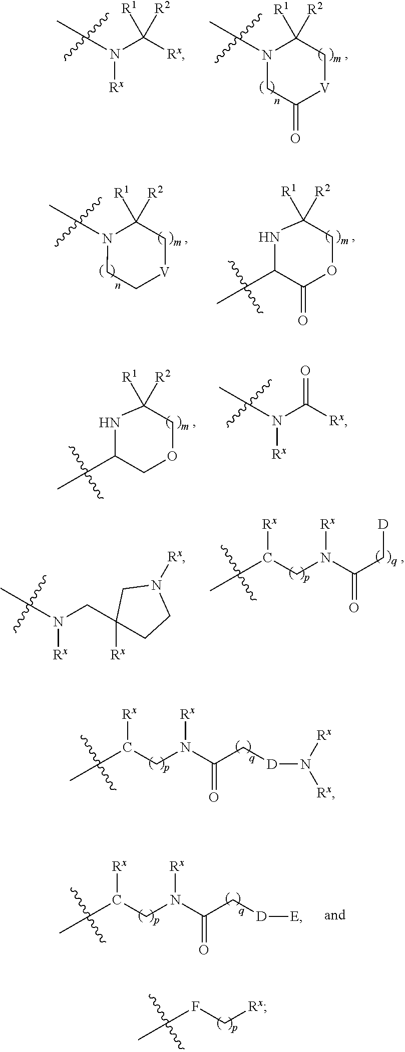
wherein
V is chosen from O, and NR;
R¹ and R² are each independently chosen from hydrogen, hydroxy groups, amino groups, linear alkyl groups, branched alkyl groups, and cyclic alkyl groups, or R¹ and R² together form a cycloalkyl group or a heterocyclic group;
each R^{x} is independently chosen from hydrogen, hydroxy groups, amino groups, sulfonyl groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups;
m, n, p, and q are independently chosen from 0, 1, 2, 3, and 4;
C, D, E, and F are chosen from hydrogen, linear alkoxy groups, branched alkoxy groups, cyclic alkoxy groups, cycloalkyl groups, heterocycloalkyl groups, aryl groups, and heteroaryl groups;
(iii) each R is independently chosen from hydrogen, hydroxy groups, amino groups, linear alkyl groups, branched alkyl groups, and cyclic alkyl groups;
(iv) R^{y} chosen from hydrogen, hydroxy groups, amino groups, linear alkyl groups, branched alkyl groups, cyclic alkyl groups, —NHC(O)alkyl groups, —NHC(O)arylalkyl groups, and —NHC(O)heteroarylalkyl groups;
(v) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}, wherein R^{z} is chosen from hydrogen, linear alkyl groups, branched alkyl groups, cyclic alkyl groups, amino groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

6. The compound of any of the preceding claims, wherein A is an aryl group.

7. The compound of claim 6, wherein A

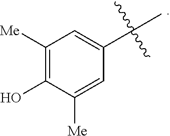 [SMILES: *c1cc(C)c(O)c(C)c1]

8. The compound of any one of claims 1-5, wherein A is an heteroaryl group.

9. The compound of any one of claims 1-5, wherein A is an alkenyl group.

10. The compound of any one of claims 1-5, wherein A is an alkenyl group.

11. The compound of any of the preceding claims, wherein B is 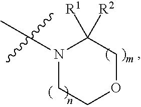
R¹ and R² are each independently chosen from hydrogen, hydroxy groups, amino groups, linear alkyl groups, branched alkyl groups, cyclic alkyl groups or together form a cycloalkyl group or a heterocyclic group; wherein the cycloalkyl group or a heterocyclic group is optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

12. The compound of claim 11, wherein B is

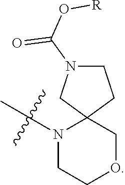

13. The compound of claim 12, wherein R is chosen from linear alkyl groups, branched alkyl groups, and cyclic alkyl groups.

14. The compound of claim 13, wherein R is t-butyl group.

15. The compound of claim 14, wherein B is

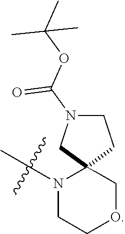

16. The compound of claim 14, wherein B is

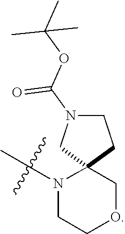

17. The compound of claim 11, wherein B is

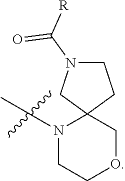

18. The compound of claim 15, wherein R is chosen from linear alkyl groups, branched alkyl groups, and cyclic alkyl groups.

19. The compound of claim 15, wherein R is chosen from aryl groups and heteroaryl groups.

20. The compound of claim 11, wherein B is

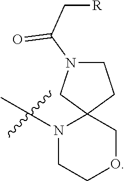

21. The compound of claim 15, wherein R is chosen from linear alkyl groups, branched alkyl groups, and cyclic alkyl groups.

22. The compound of claim 15, wherein R is chosen from aryl groups and heteroaryl groups.

23. The compound of any of claims 1-10, wherein B is 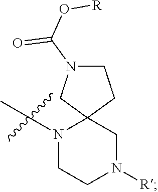
R is chosen from linear alkyl groups, branched alkyl groups, cyclic alkyl groups, aryl groups, and heteroaryl groups; and R′ is chosen from linear alkyl groups, branched alkyl groups, cyclic alkyl groups, —C(O)—C₁-C₆ linear alkyl groups, —C(O)—C₃-C₆ branched alkyl groups, and —C(O)—C₃-C₆cyclic alkyl groups.

24. The compound of claim 24, wherein B chosen from

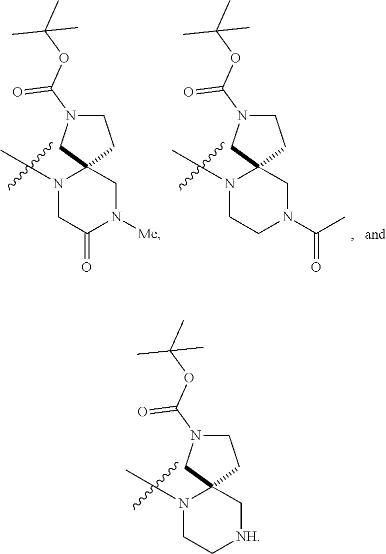

25. The compound of any of claims 1-10, wherein B is 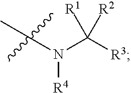
R¹ and R² are each independently chosen from hydrogen, hydroxy groups, linear alkyl groups, branched alkyl groups, and cyclic alkyl groups, or R¹ and R² together form a cycloalkyl group or a heterocyclic group;
R³ and R⁴ are each independently chosen from hydrogen, hydroxy groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups.

26. The compound of claim 25, wherein B is chosen from

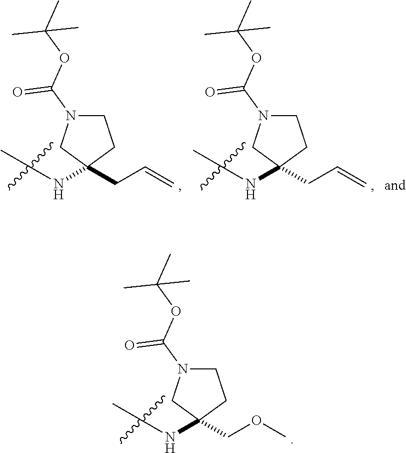

27. The compound of any of claims 1-10, wherein B is 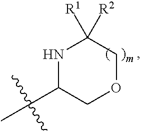
wherein m is 0 or 1; R¹ and R² are each independently chosen from hydrogen, hydroxy groups, linear alkyl groups, branched alkyl groups, cyclic alkyl groups. or R¹ and R² together form a cycloalkyl group or a heterocyclic group.

28. The compound of claim 27, wherein B is chosen from

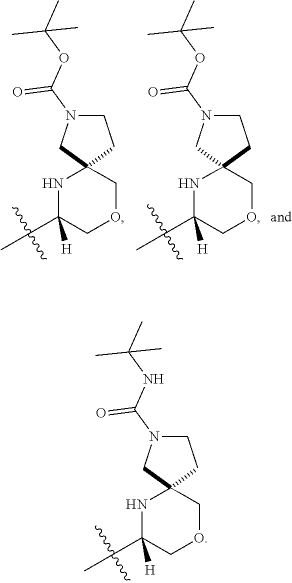

29. The compound of any of claims 1-10, wherein B is 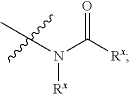
wherein each R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups.

30. The compound of any of claims 1-10, wherein B is 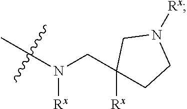
each R^{x} is independently chosen from hydrogen, hydroxy groups, linear alkyl groups, branched alkyl groups, cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups.

31. The compound of any of claims 1-10, wherein B is 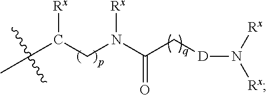
each R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups; p and q are independently chosen from 0, 1, 2, 3, and 4; C and D are independently chosen from linear alkoxy groups, branched alkoxy groups, cyclic alkoxy groups, cycloalkyl groups, heterocycloalkyl groups, aryl groups, and heteroaryl groups.

32. The compound of claim 29, wherein B is

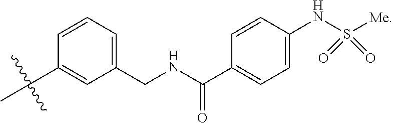 [SMILES: *c1cccc(CNC(=O)c2ccc(NS(C)(=O)=O)cc2)c1]

33. The compound of any of claims 1-10, wherein B is 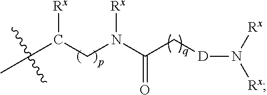
each R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups; p and q are independently chosen from 0, 1, 2, 3, and 4; C, D, and E are independently chosen from hydrogen, linear alkoxy groups, branched alkoxy groups, cyclic alkoxy groups, cycloalkyl groups, heterocycloalkyl groups, aryl groups, and heteroaryl groups.

34. The compound of claim 33, wherein B is chosen from

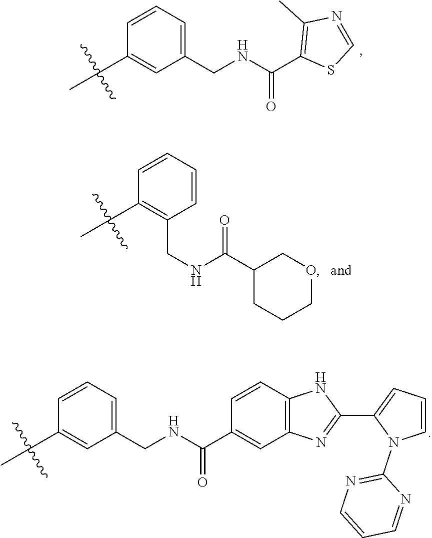 [SMILES: *c1cccc(CNC(=O)c2scnc2C)c1 ; *c1ccccc1CNC(=O)C1CCCOC1 ; *c1cccc(CNC(=O)c2ccc3[nH]c(-c4cccn4-c4ncccn4)nc3c2)c1]

35. The compound of any of claims 1-10, wherein B 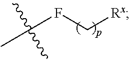
R^{x} is independently chosen from hydrogen, hydroxy groups, linear alkyl groups, branched alkyl groups, and cyclic alkyl groups, linear alkoxy groups, branched alkoxy groups, cyclic alkoxy groups, linear alkylene groups, branched alkylene groups, cyclic alkylene groups, heterocycloalkyl groups, aryl groups, and heteroaryl groups; and F is chosen from linear alkoxy groups, branched alkoxy groups, cyclic alkoxy groups, cycloalkyl groups, heterocycloalkyl groups, aryl groups, and heteroaryl groups.

36. The compound of claim 33, wherein B is

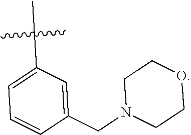 [SMILES: *c1cccc(CN2CCOCC2)c1]

37. The compound of any of the preceding claims, wherein one of X⁷ and X⁸ is chosen from hydrogen, amino groups, —NHC(O)alkylgroups, —NHC(O)arylalkylgroups, and —NHC(O)heteroarylalkylgroups.

38. The compound of any of the preceding claims, wherein one of X¹ and X² chosen from —NH₂, —NHC(O)CH₃, and

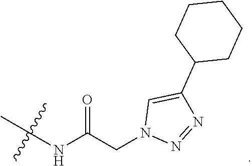 [SMILES: *NC(=O)Cn1cc(C2CCCCC2)nn1]

39. The compound of any of one of claims 1-29, wherein Z is hydrogen.

40. The compound of any of one of claims 1-29, wherein Z is —CN.

41. The compound of any of one of claims 1-29, wherein Z is —CO₂H.

42. The compound of any of one of claims 1-29, wherein Z is —C(O)R^{z}, —CO₂R^{z}, or —C(O)NHSO₂R^{z}; wherein R^{z} is chosen from hydrogen, linear alkyl groups, branched alkyl groups, cyclic alkyl groups, carbocyclic groups, amino groups, heterocyclic groups, aryl groups, and heteroaryl groups.

43. The compound of any of one of claims 1-29, wherein Z is —C(O)NHCN.

44. The compound of claim 1, wherein the compound is chosen from: 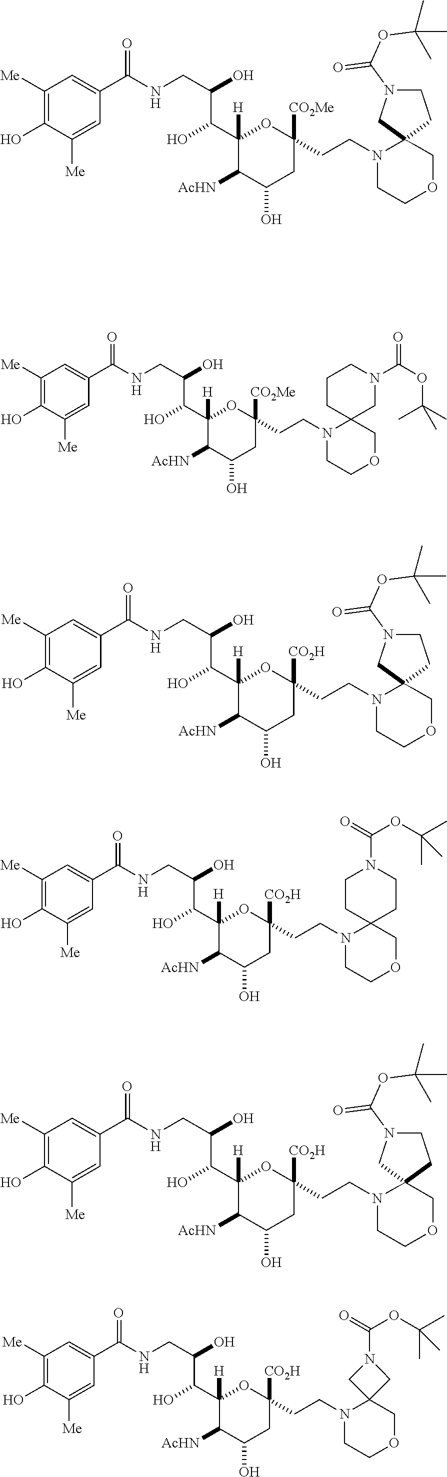 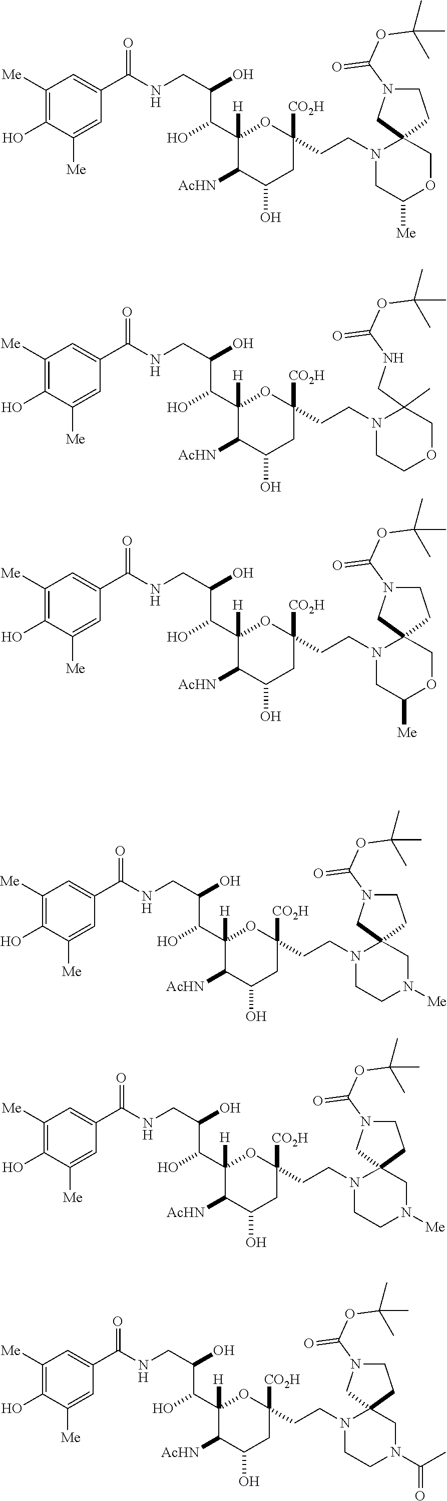 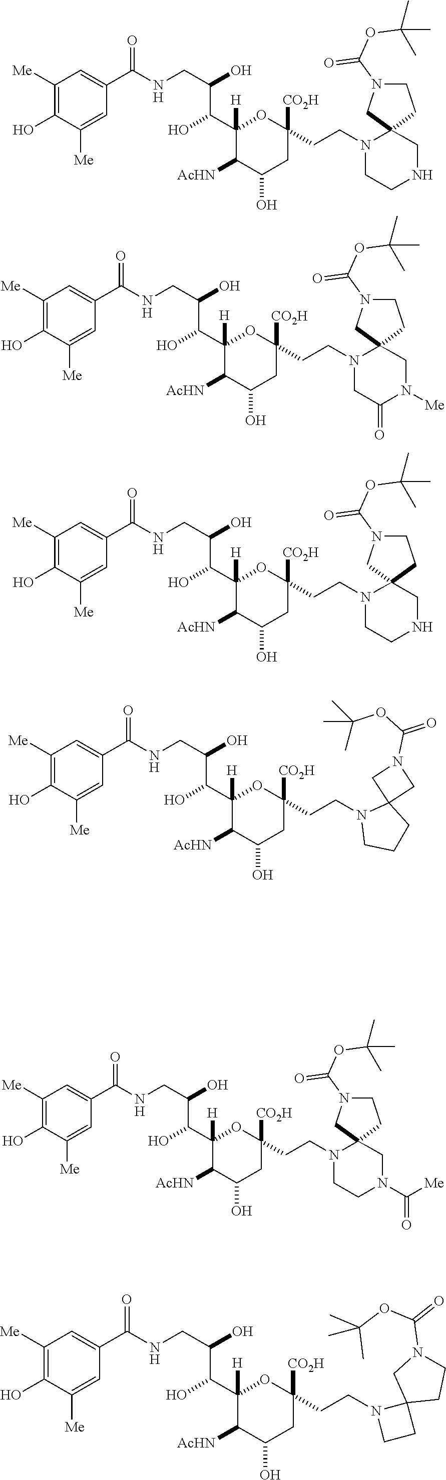 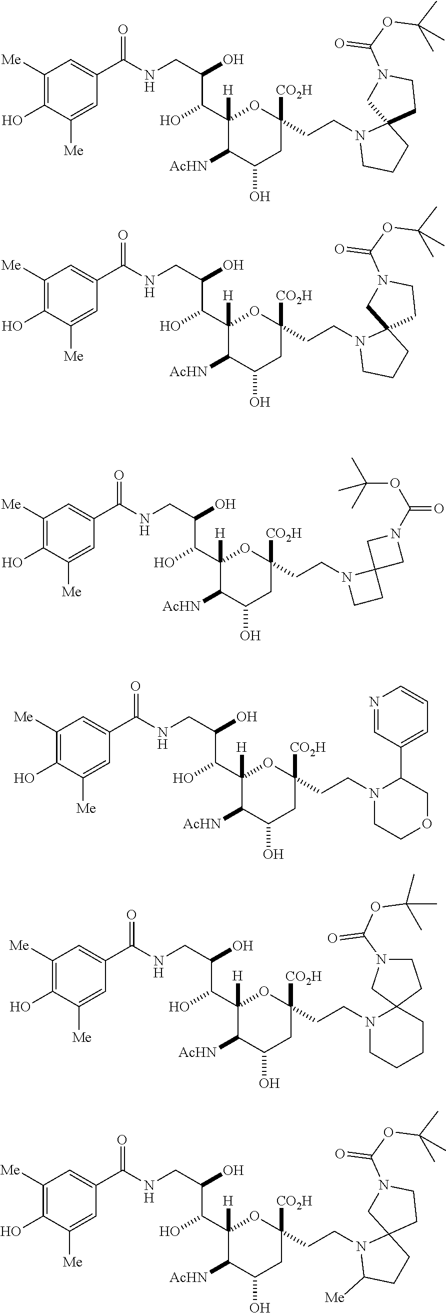 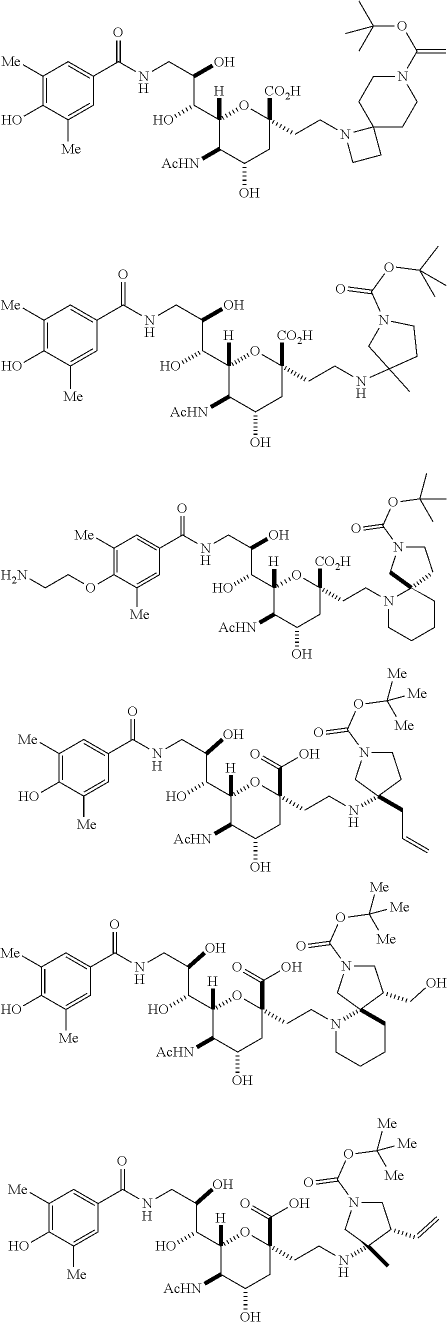 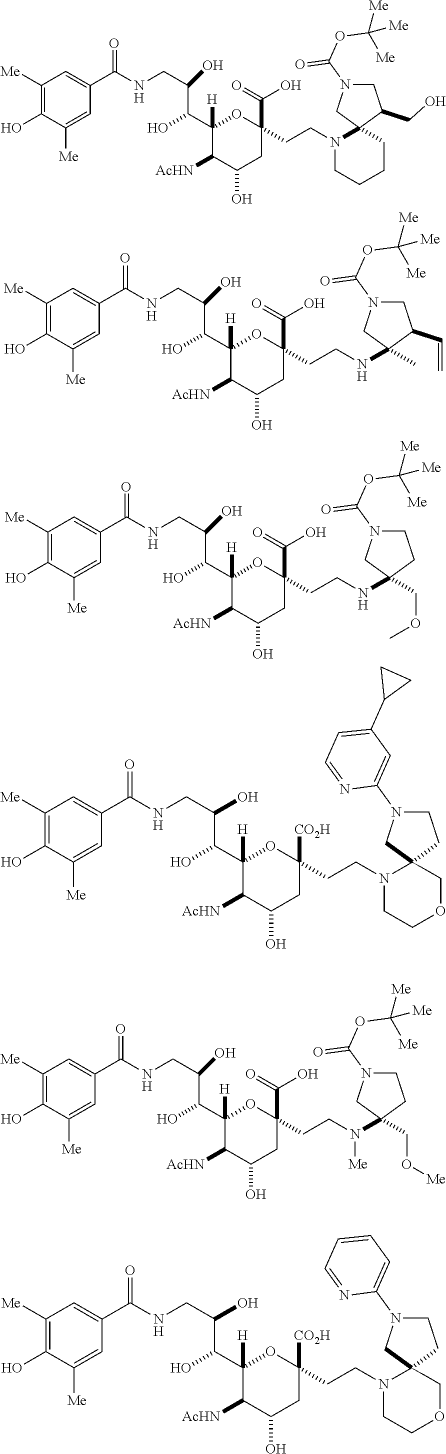 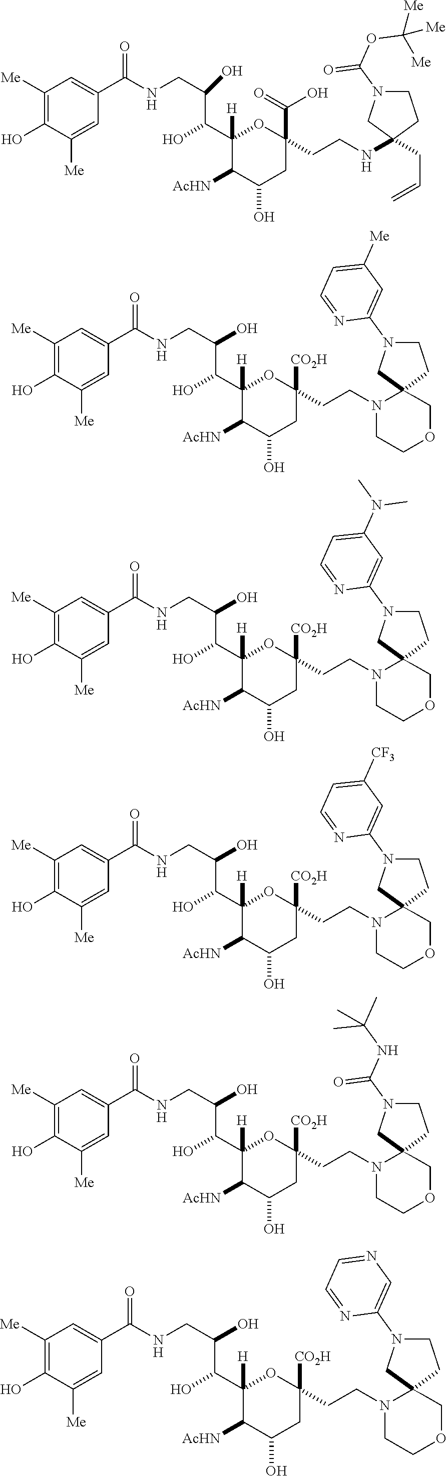 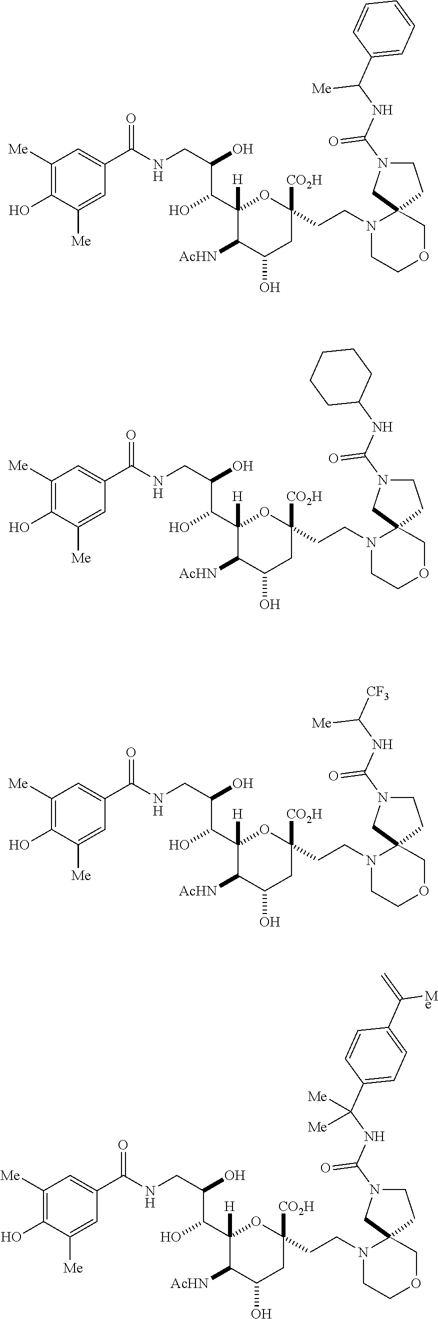 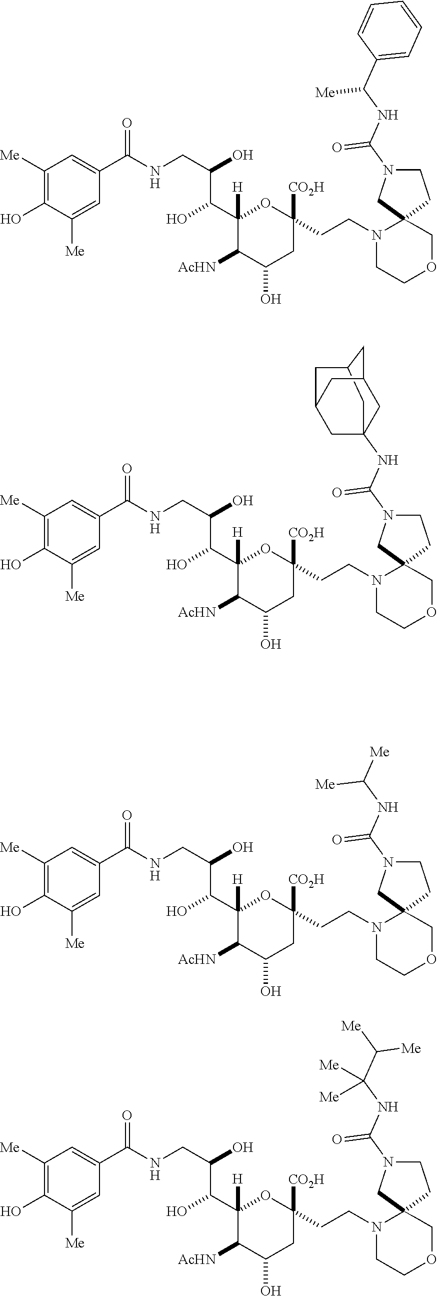 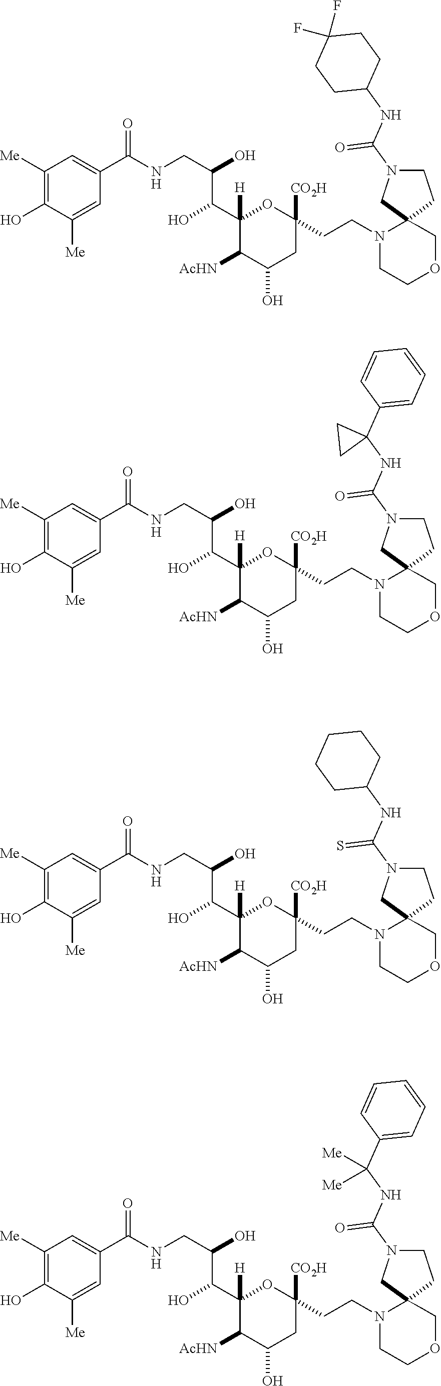 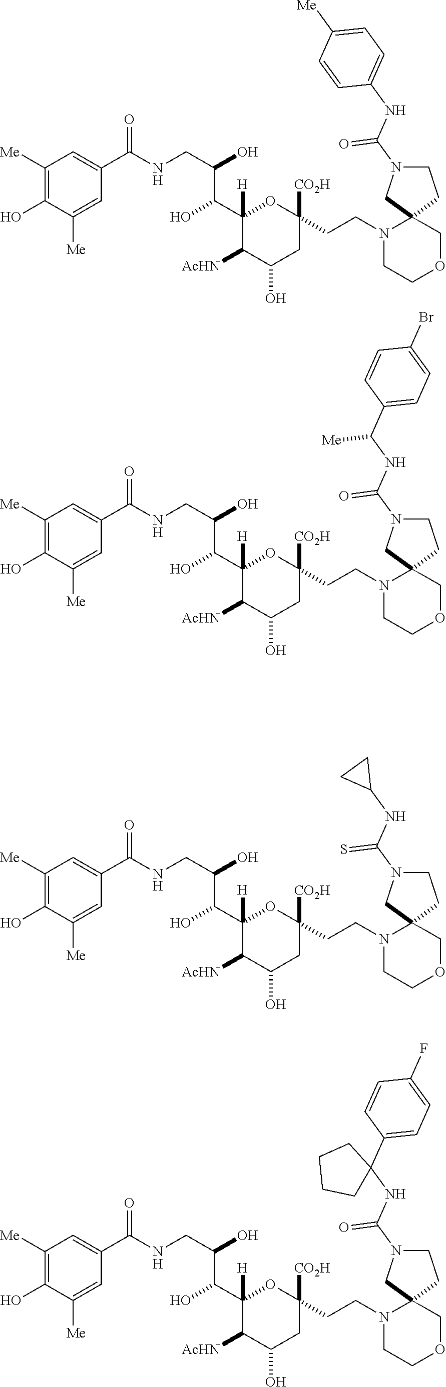 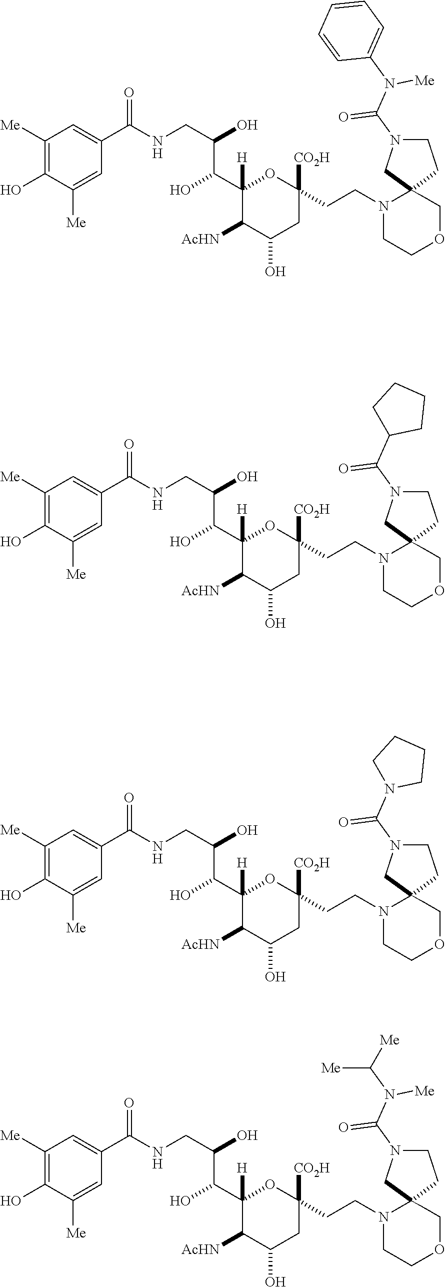 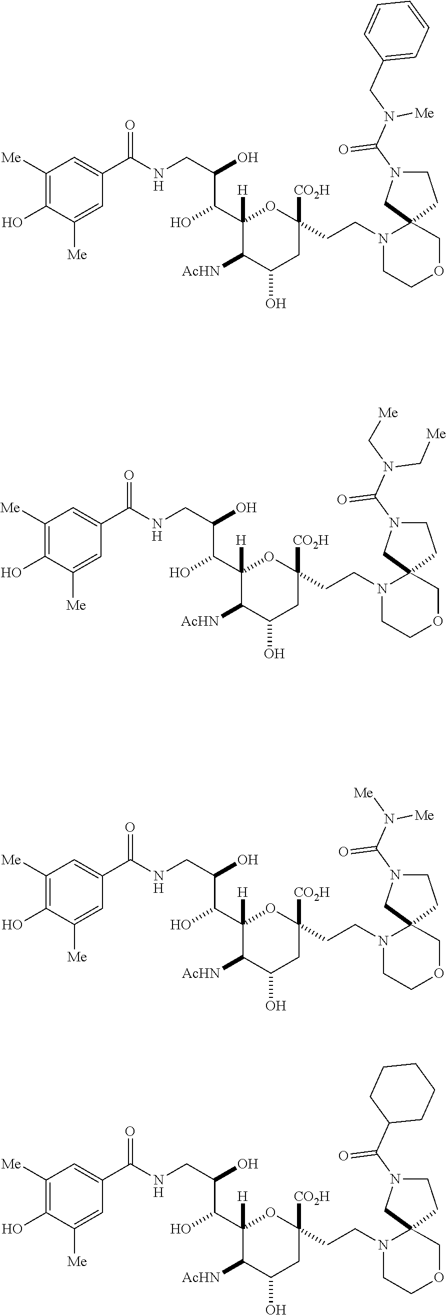 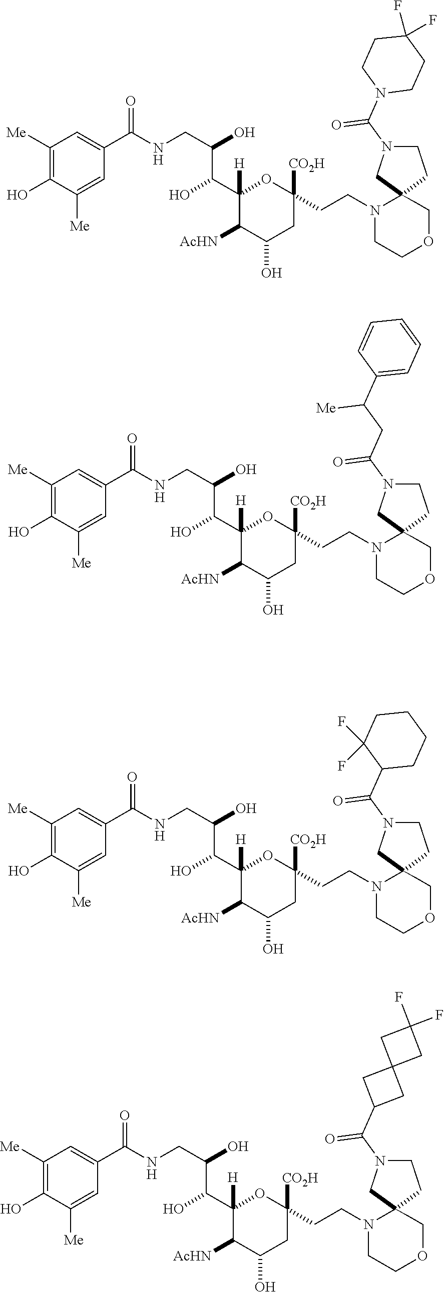 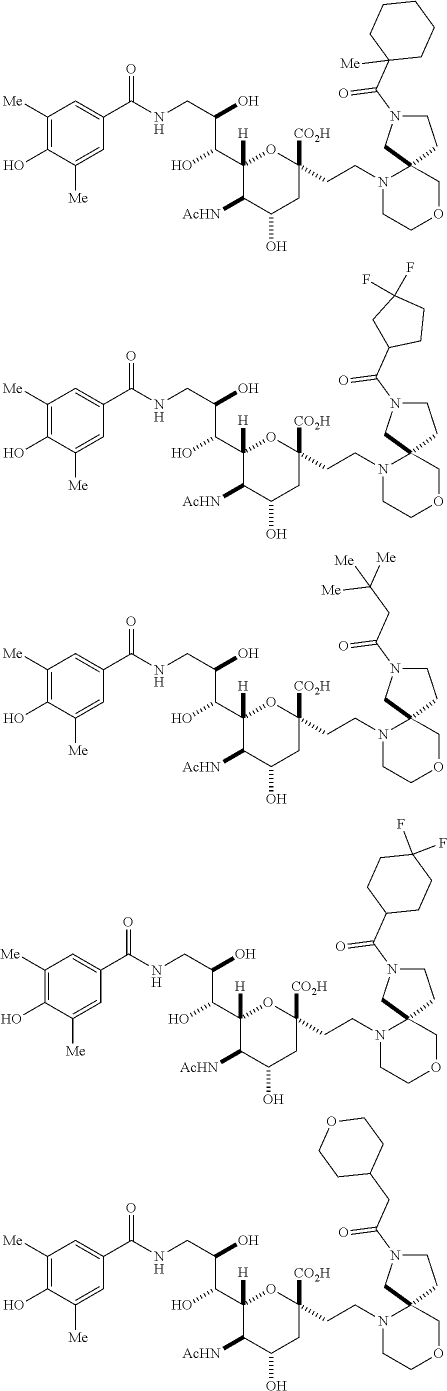 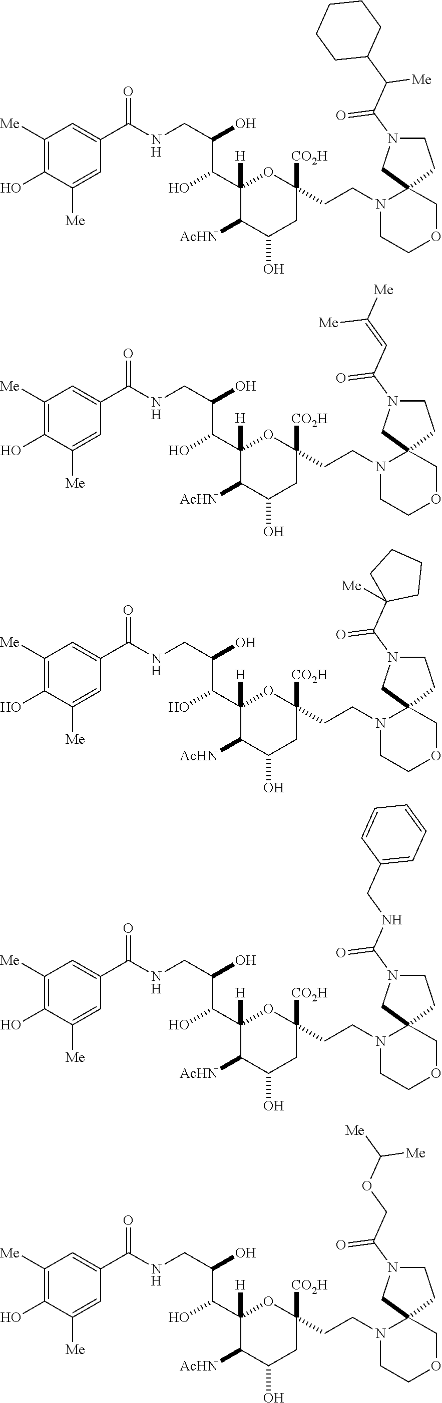 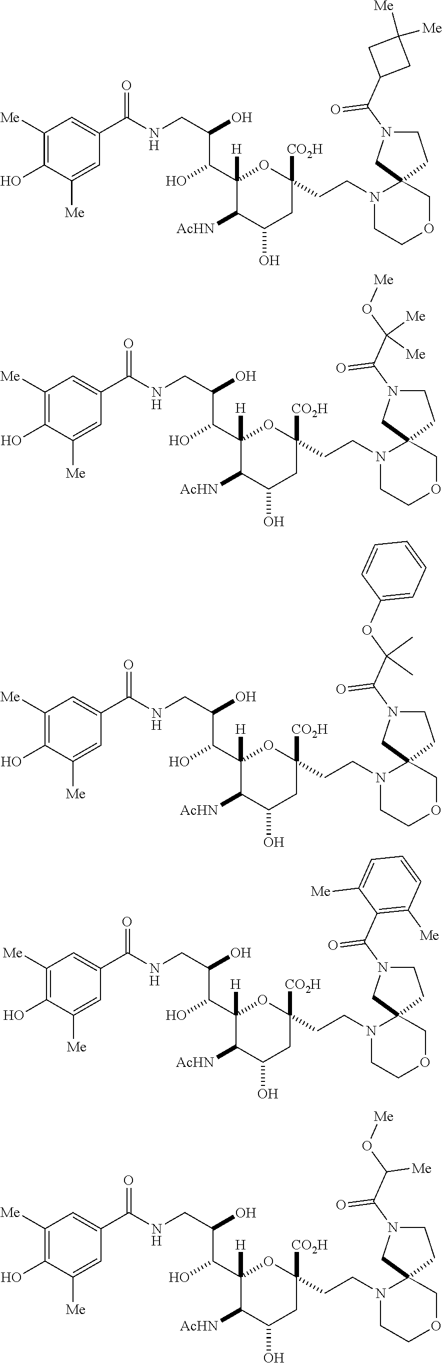 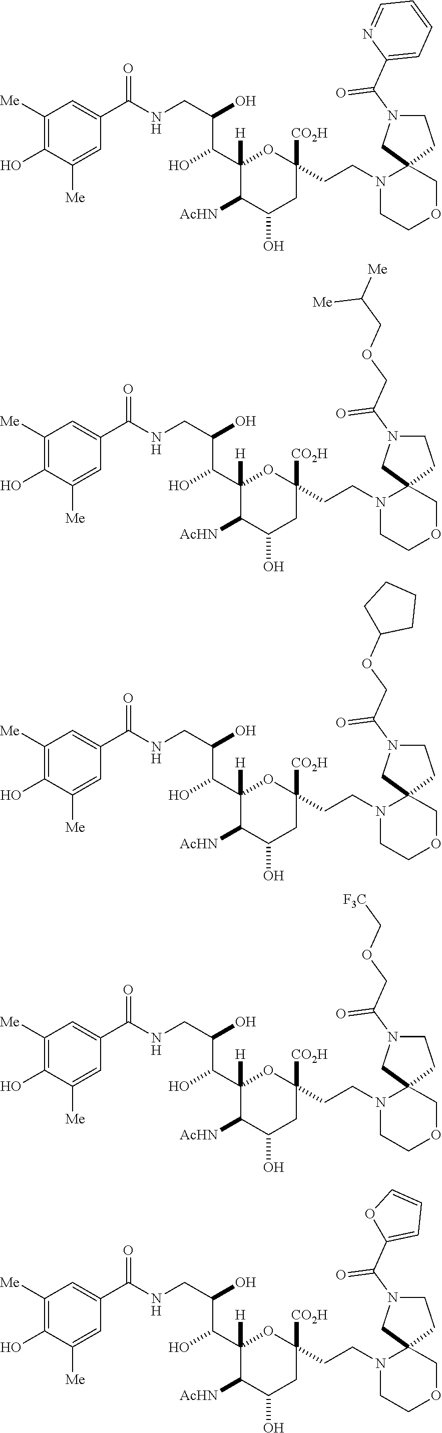 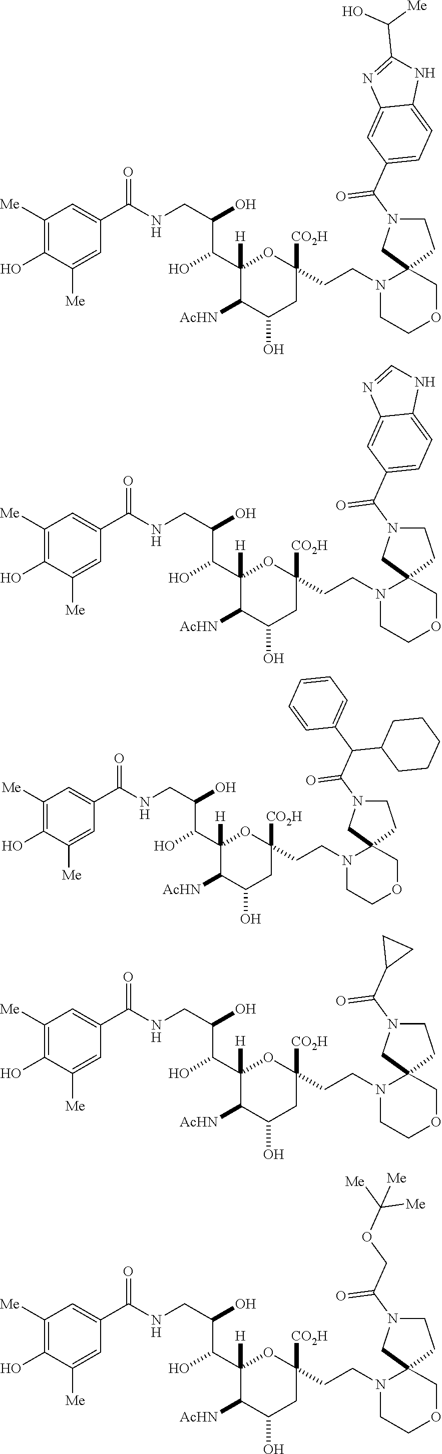 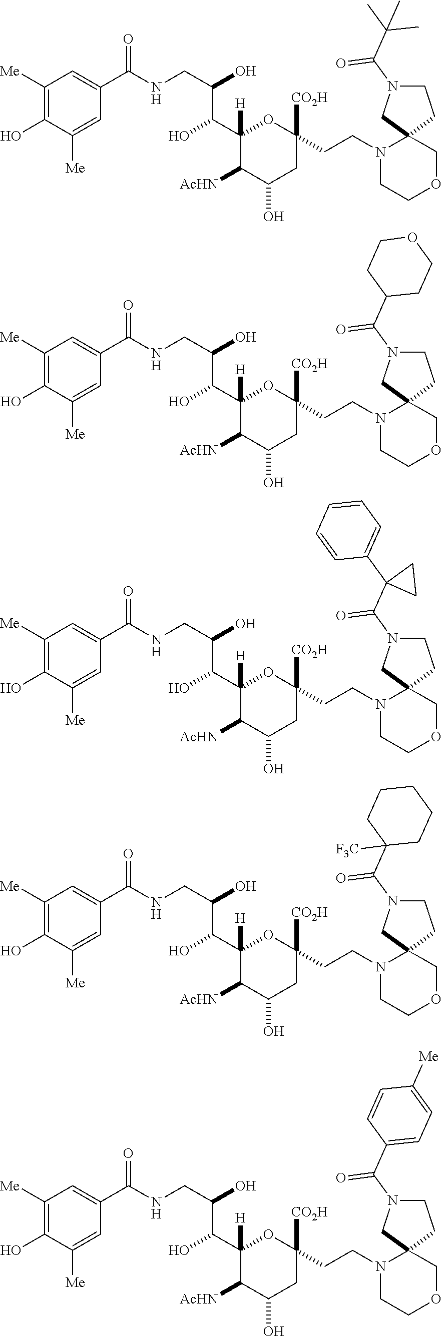 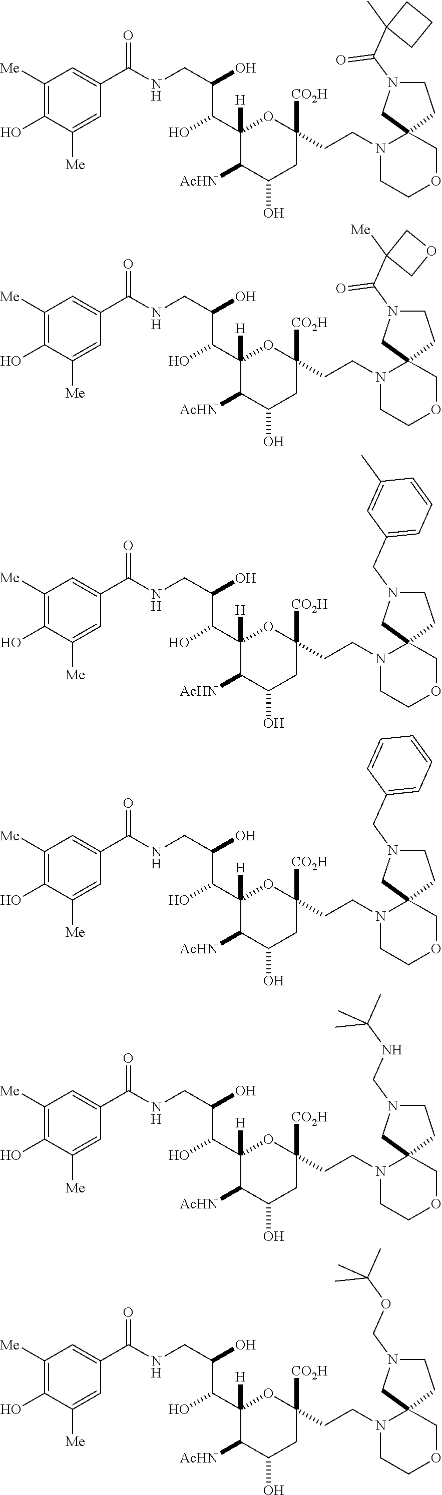 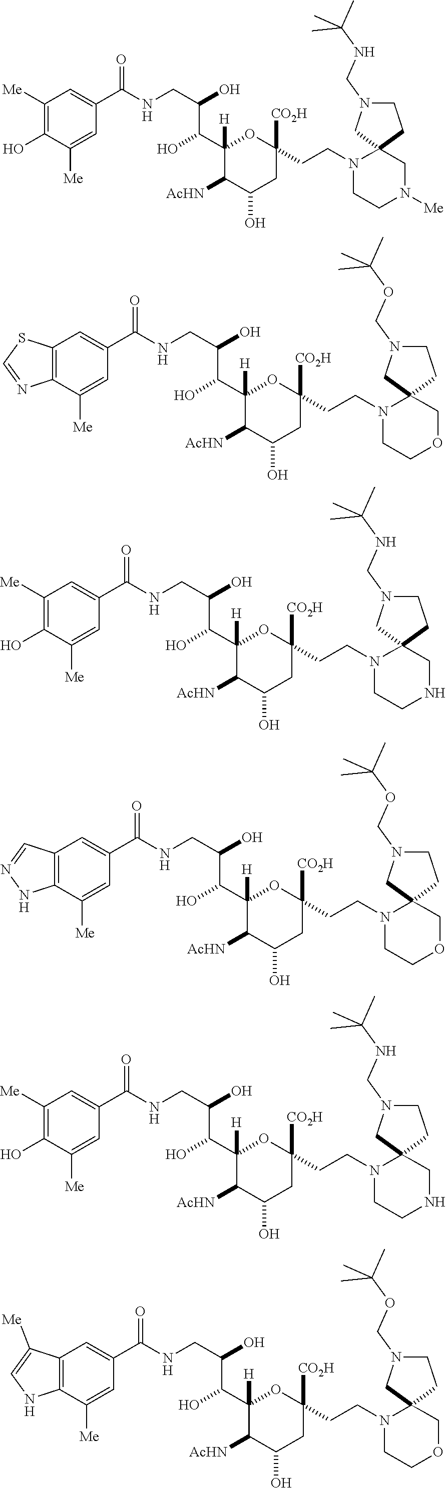 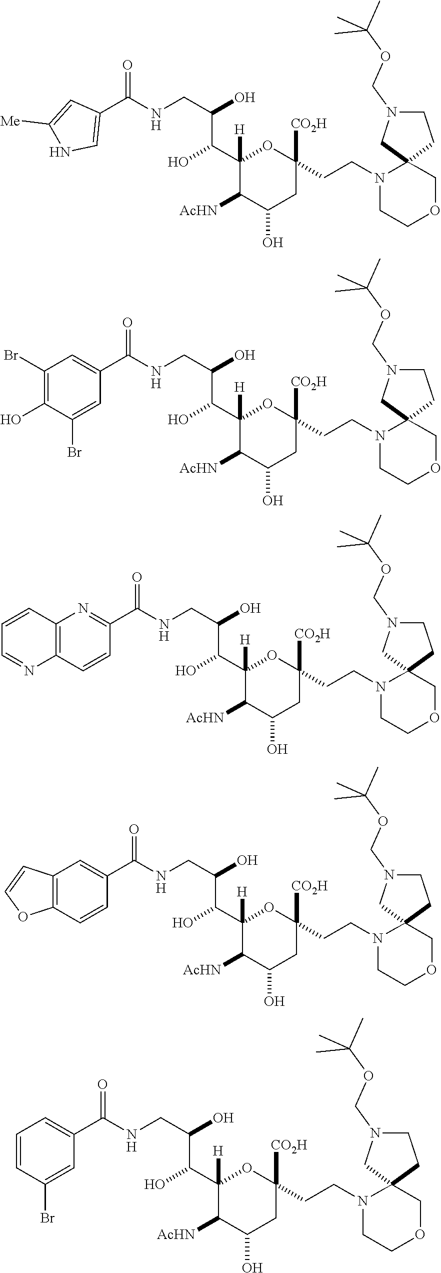 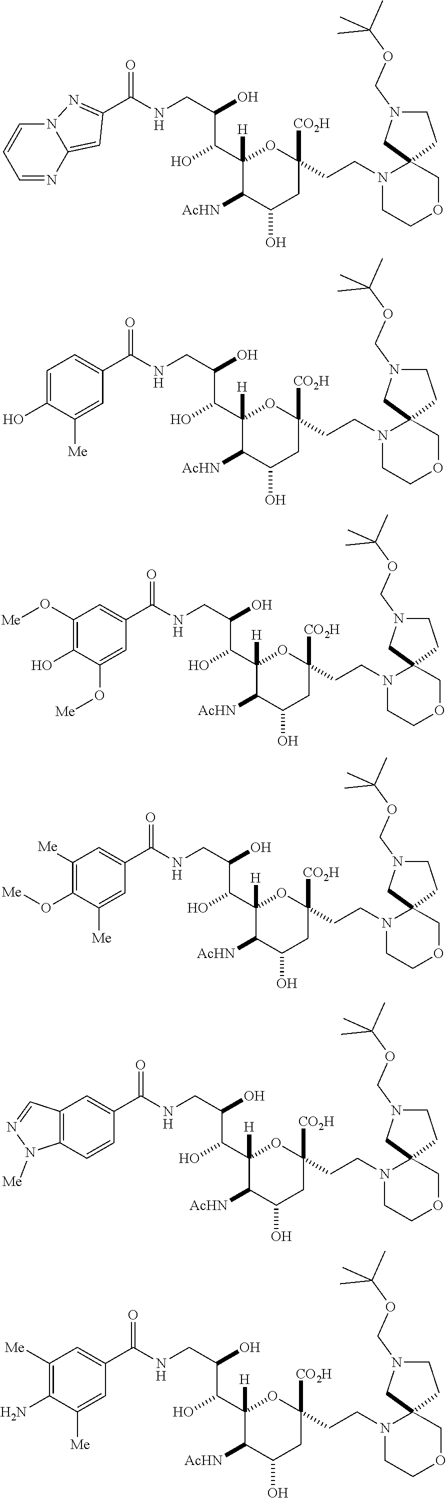 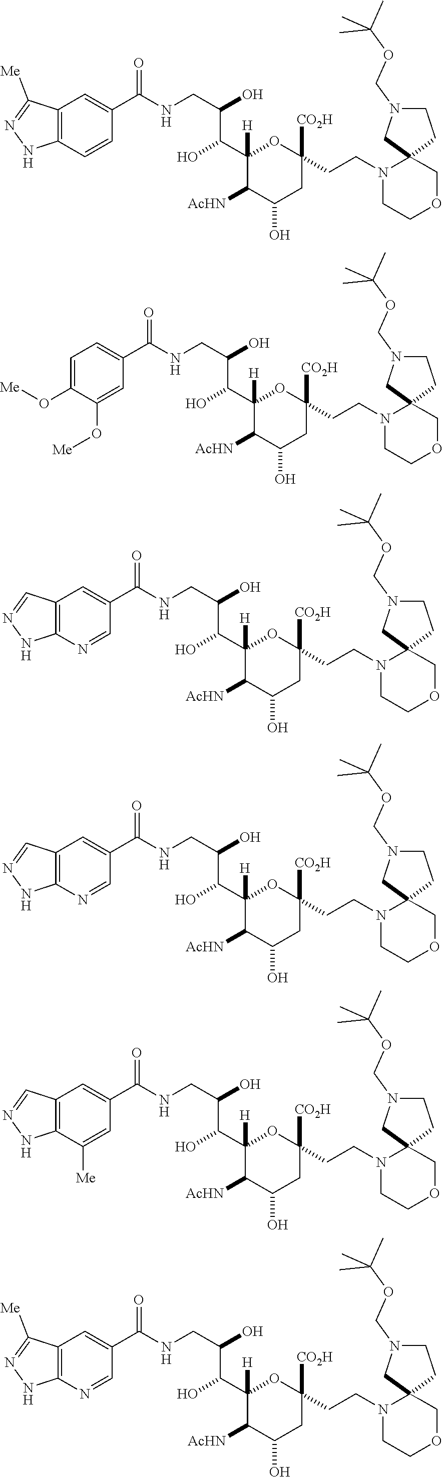 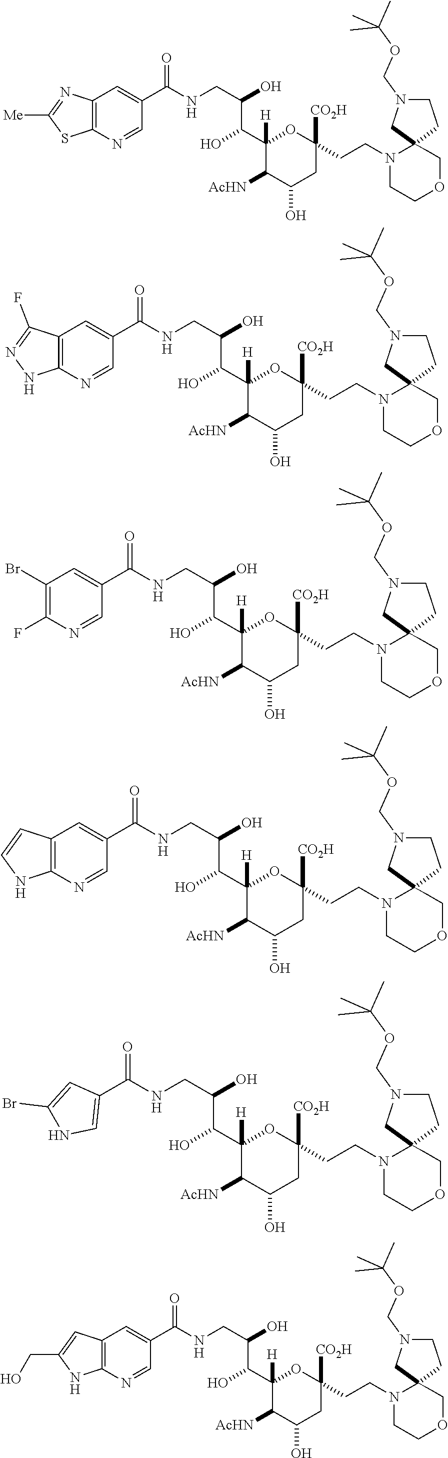 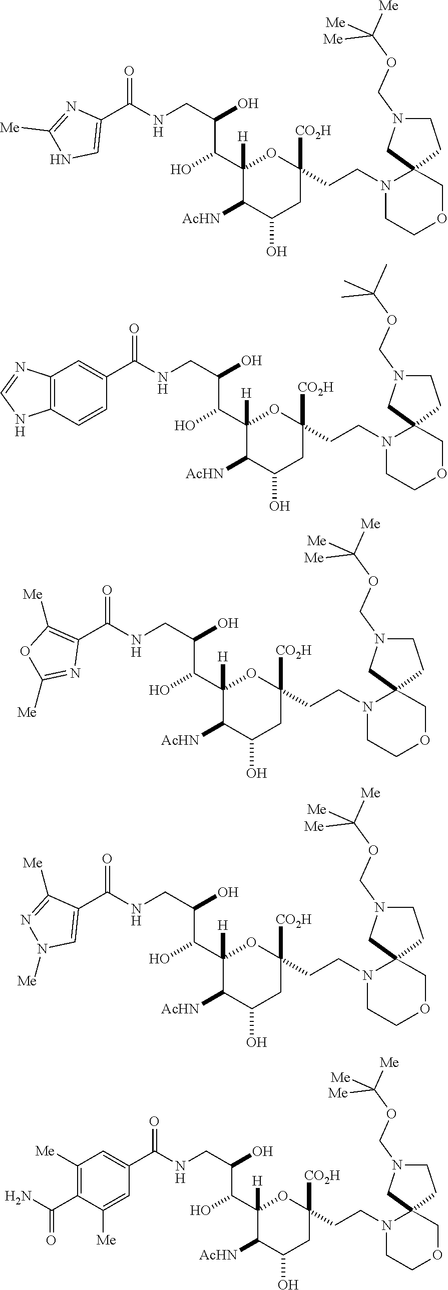 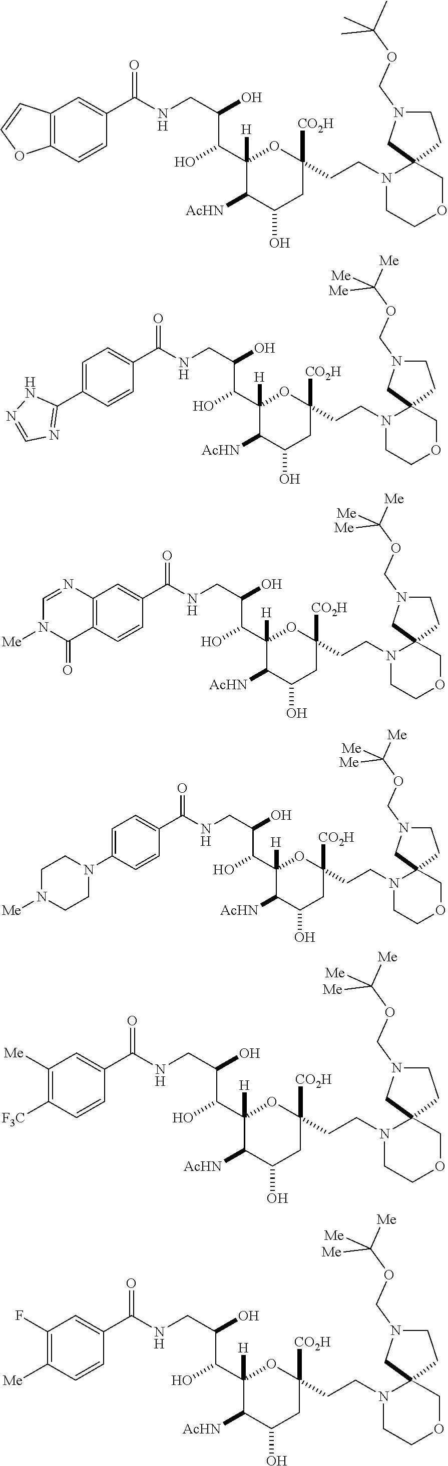 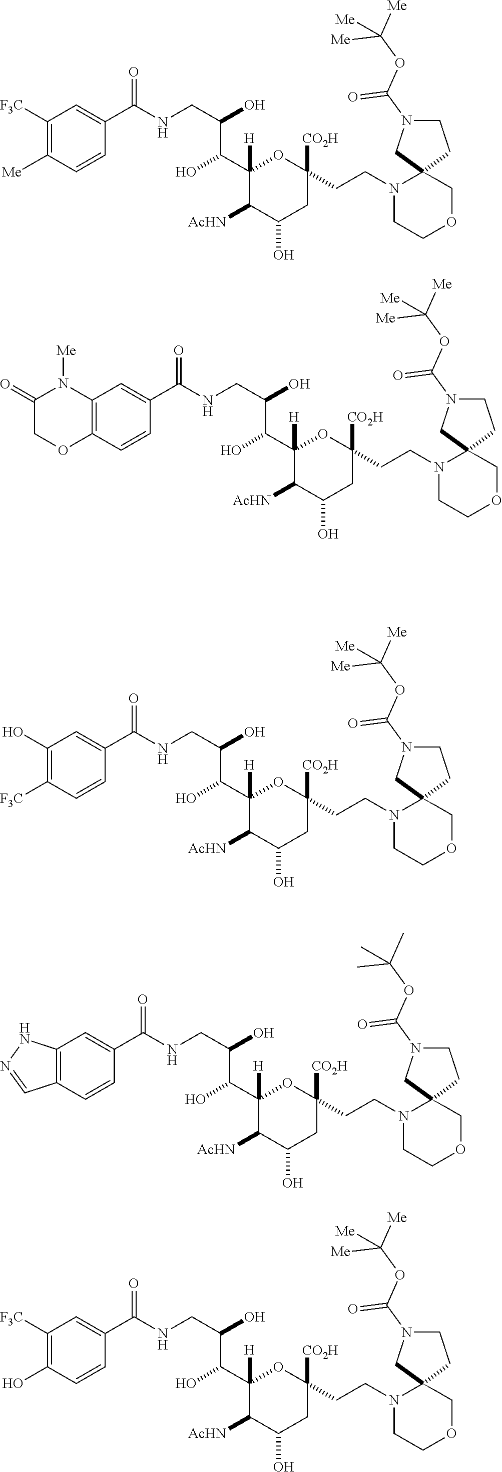 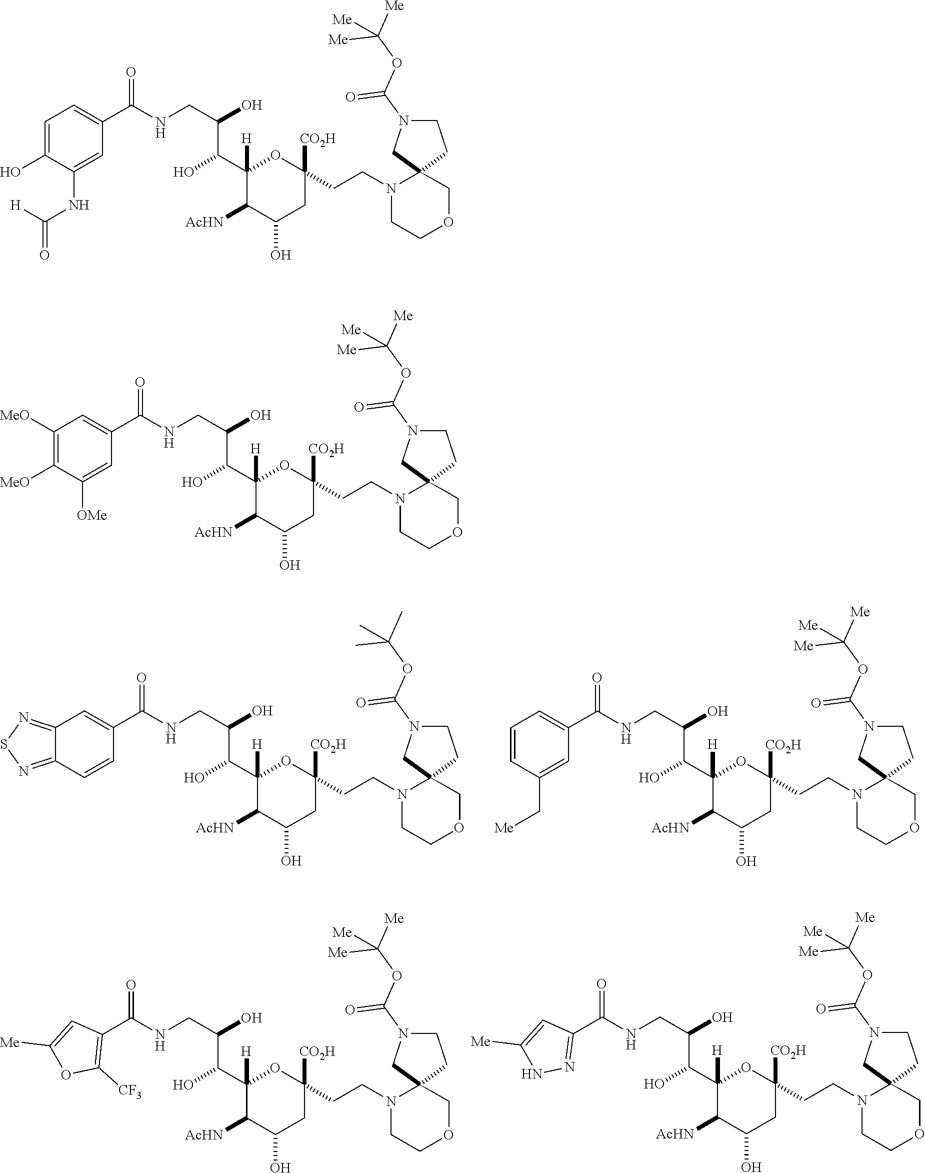 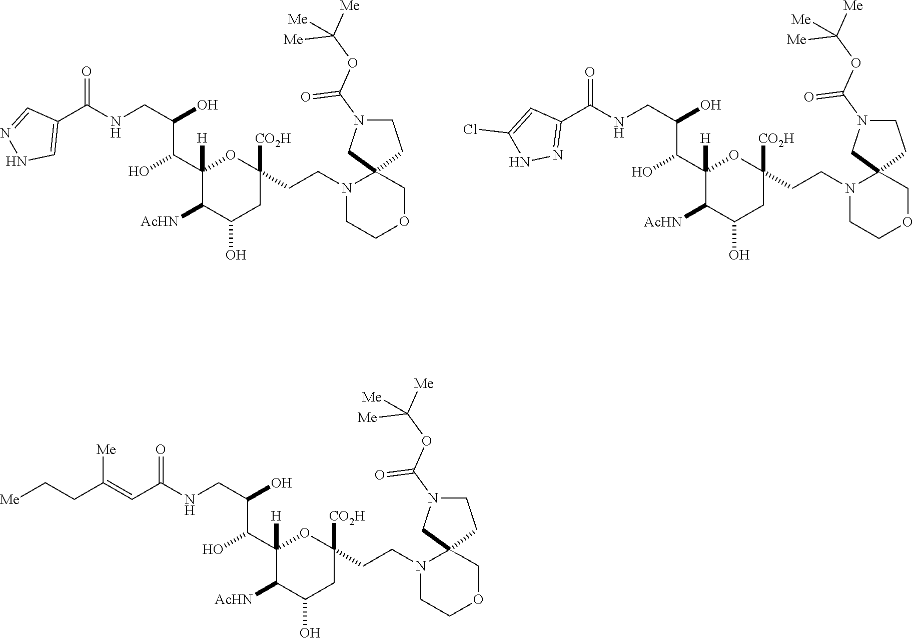 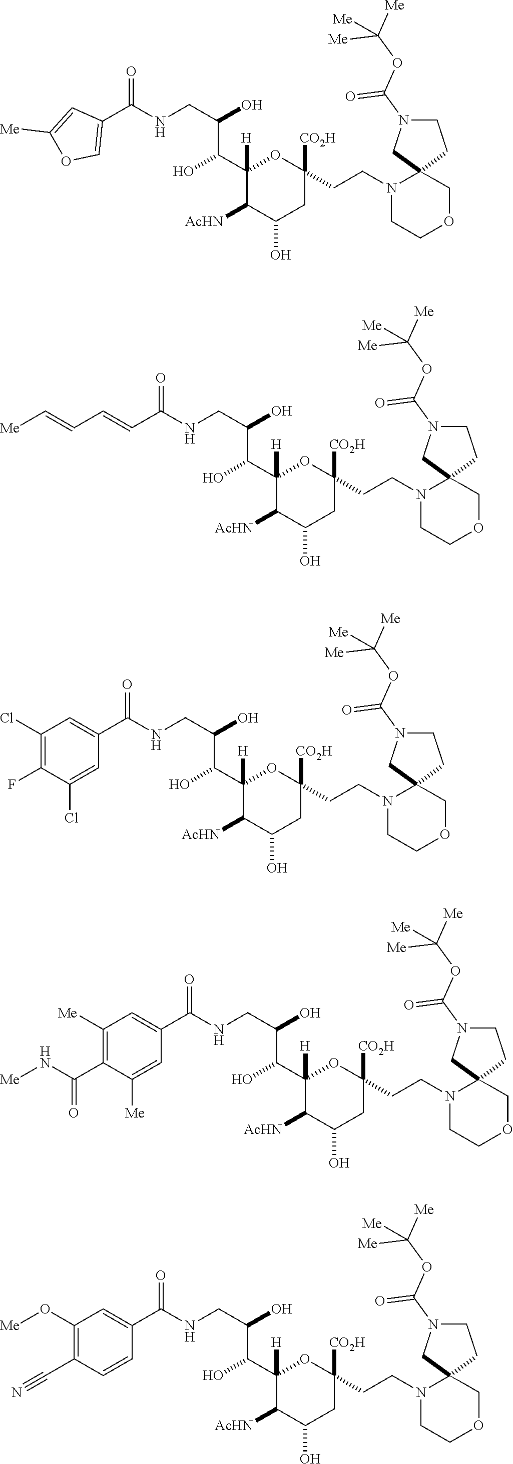 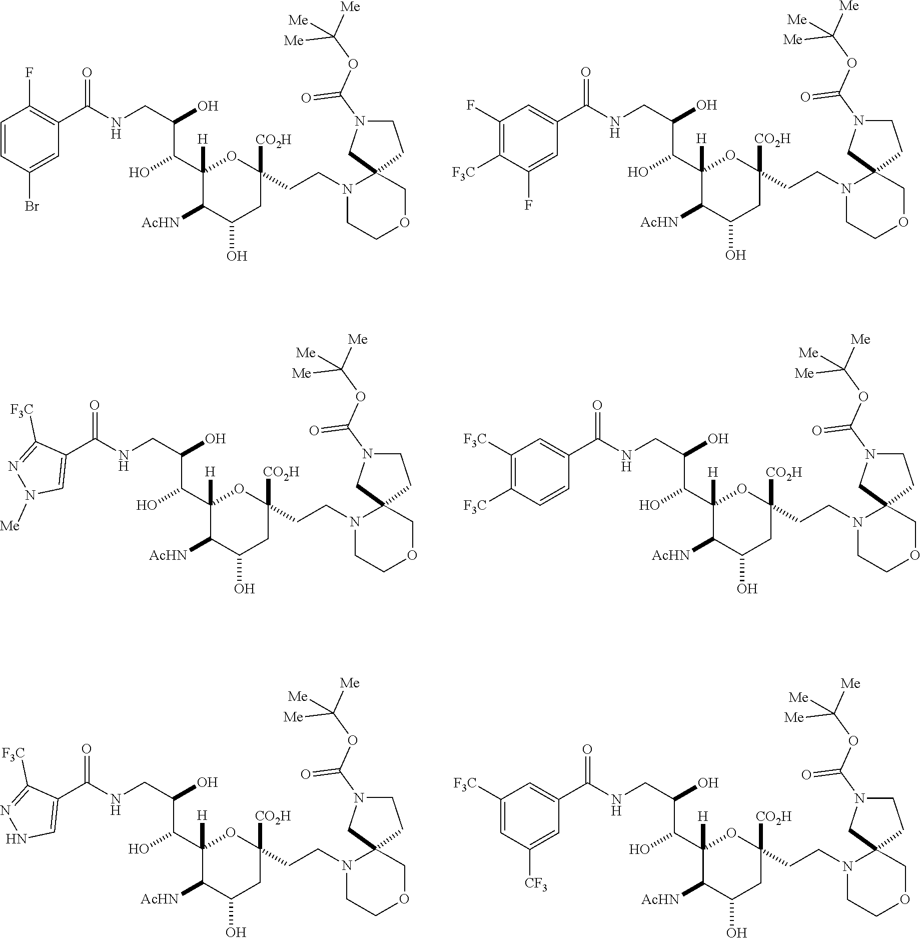 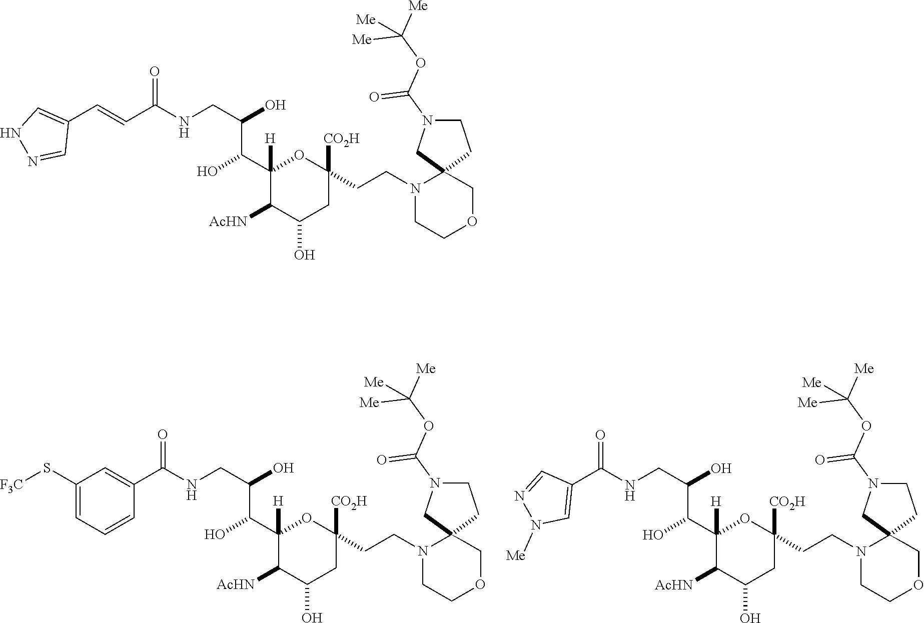 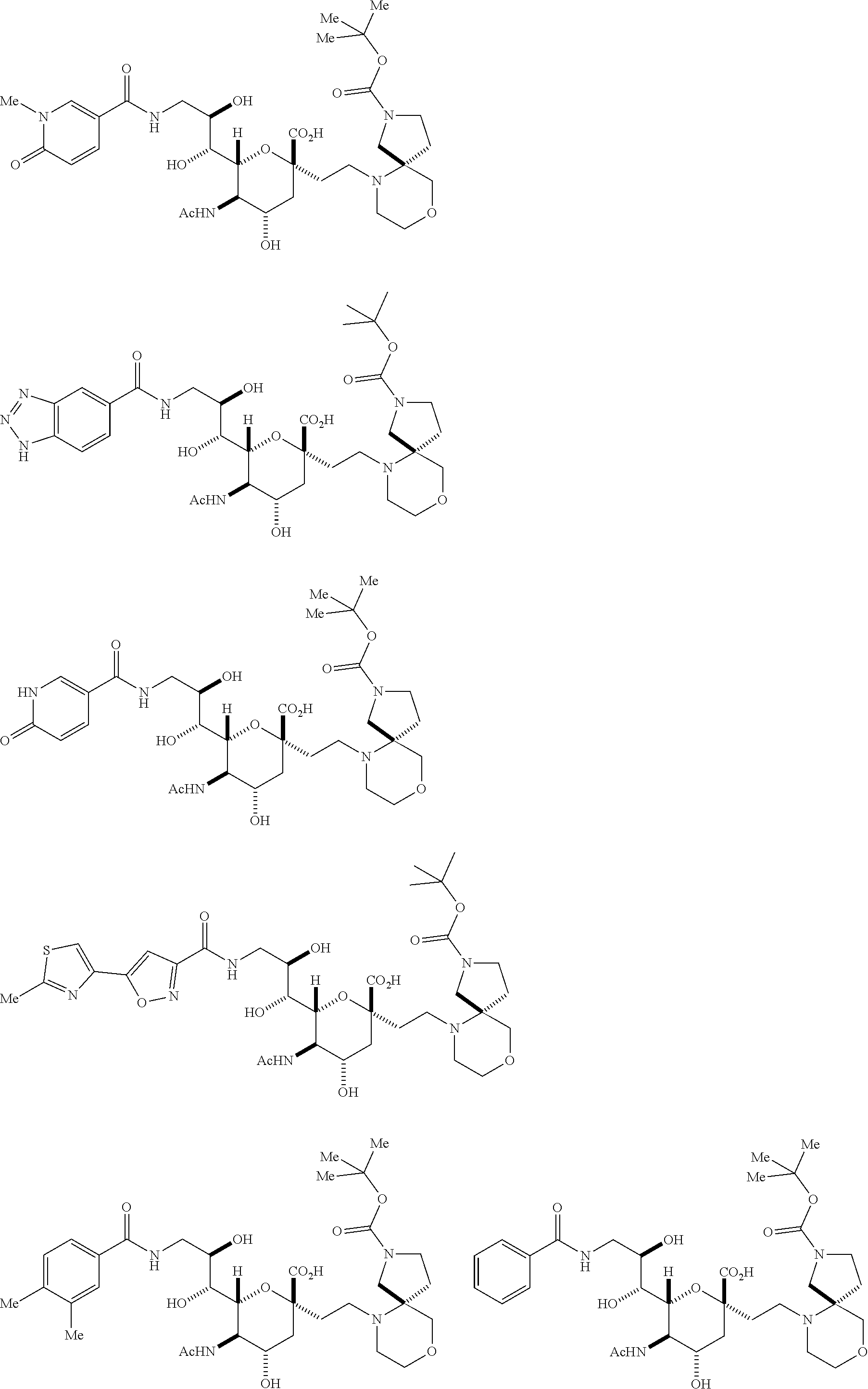 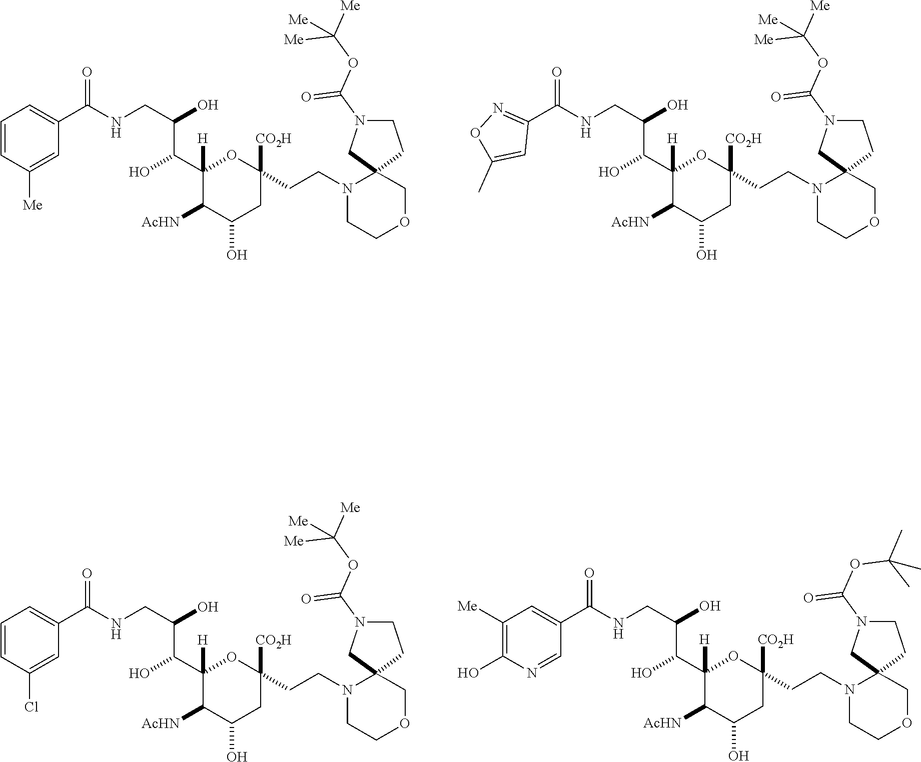 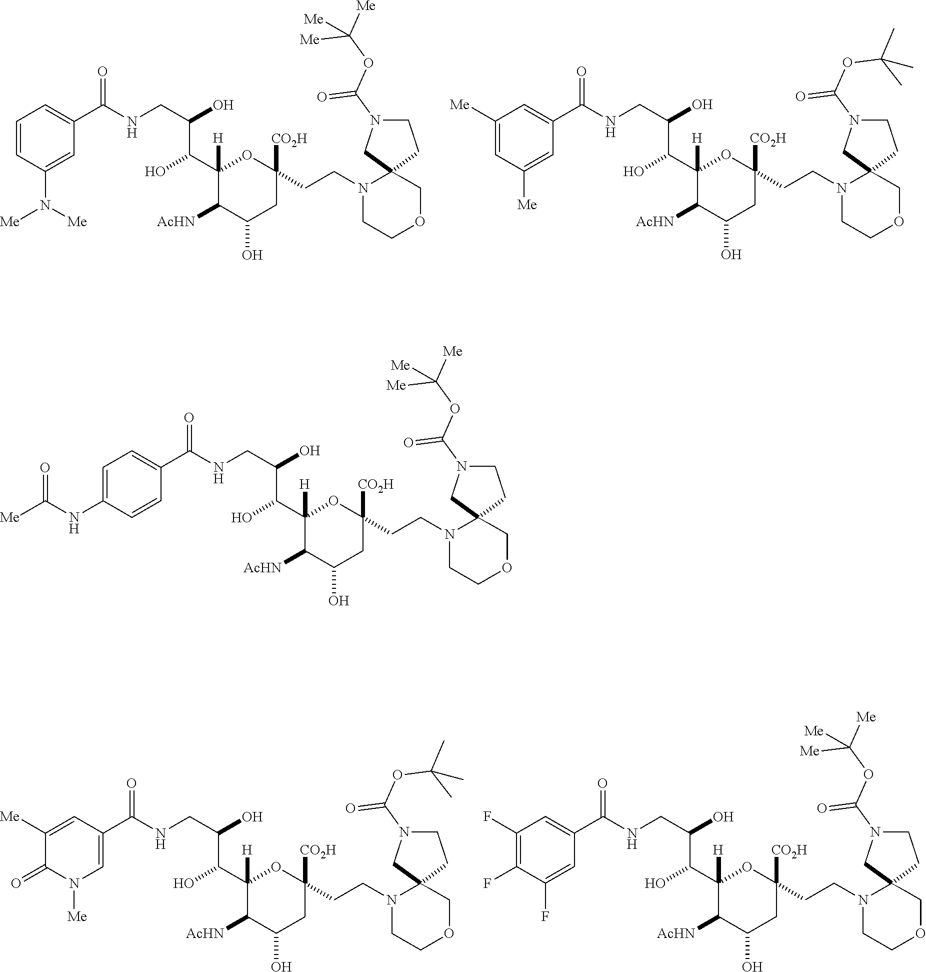 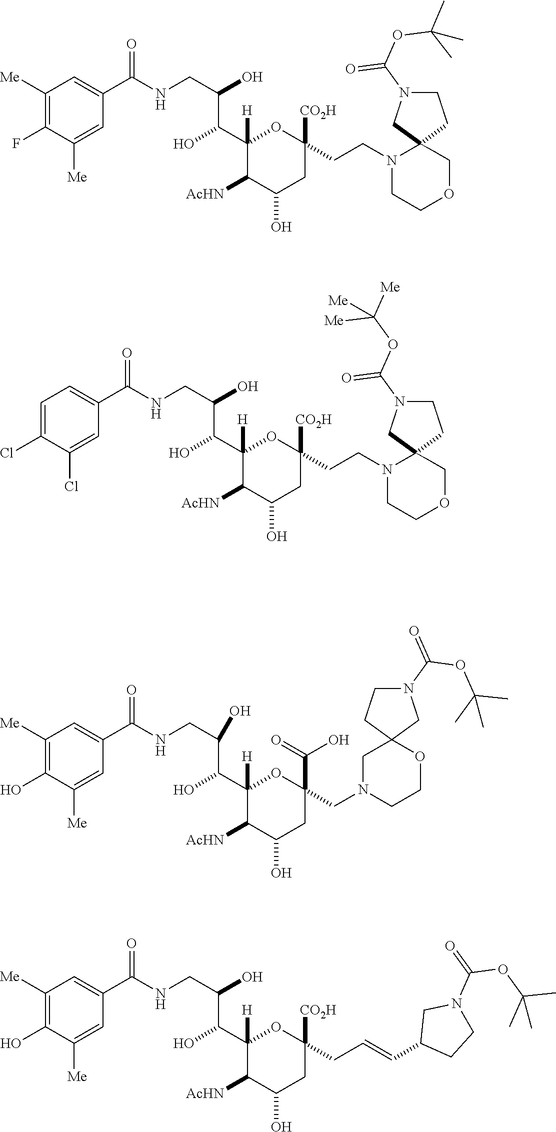 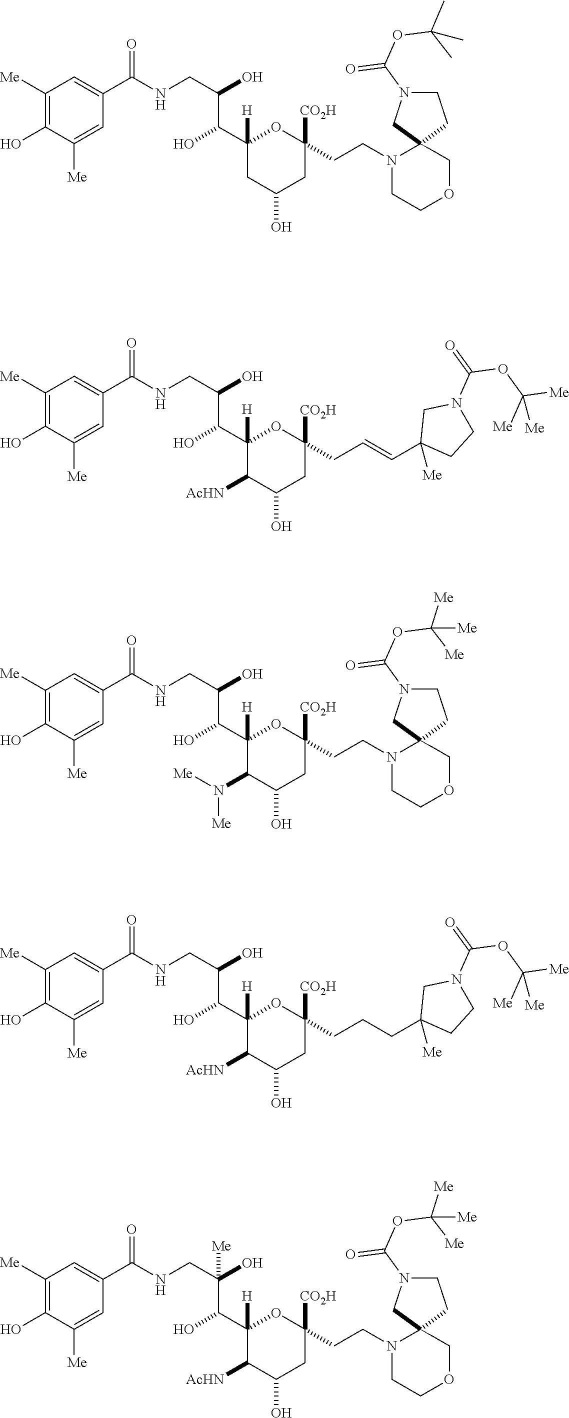 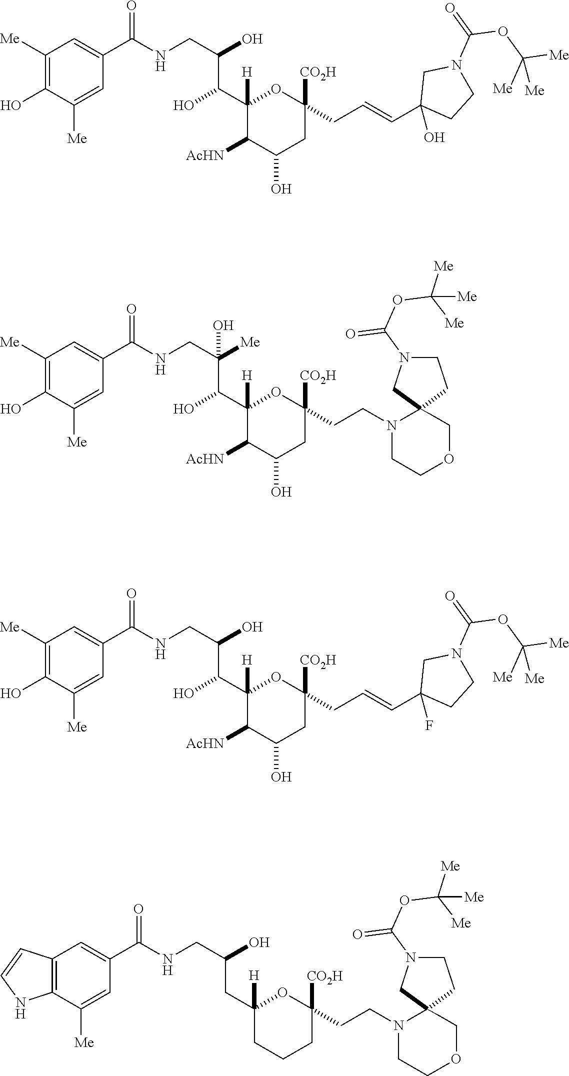 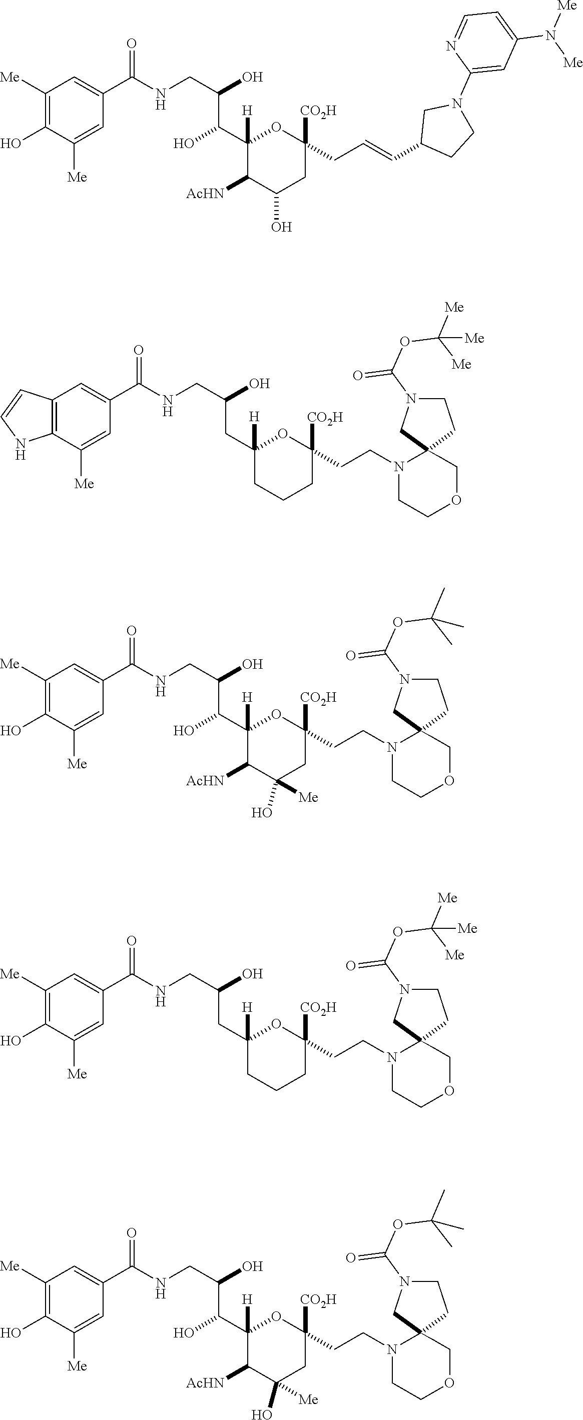 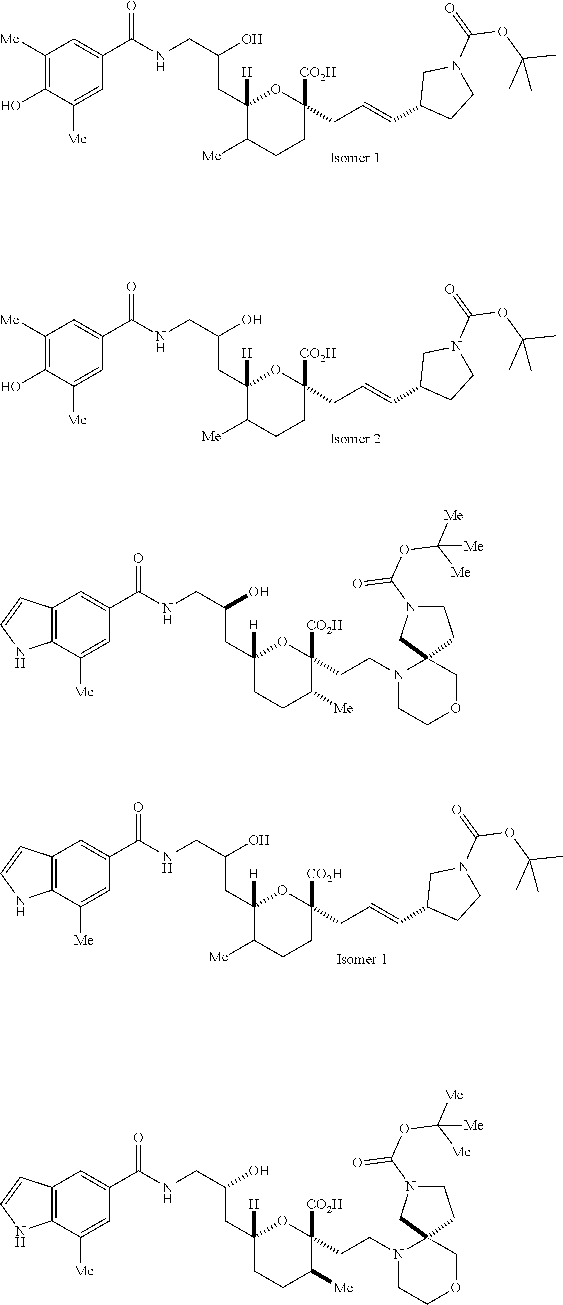 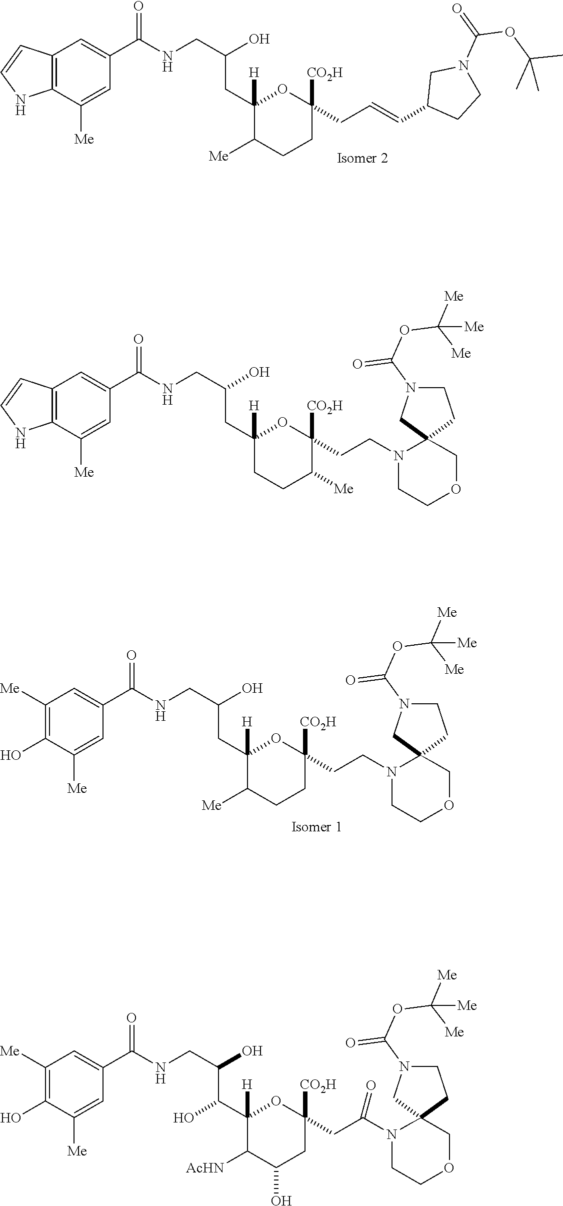 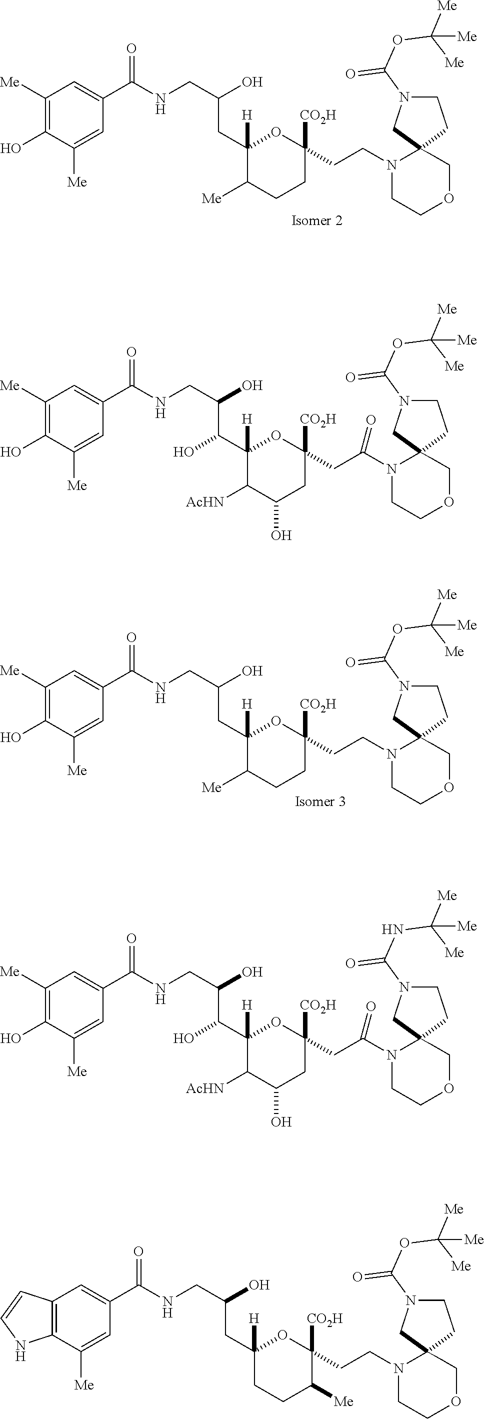 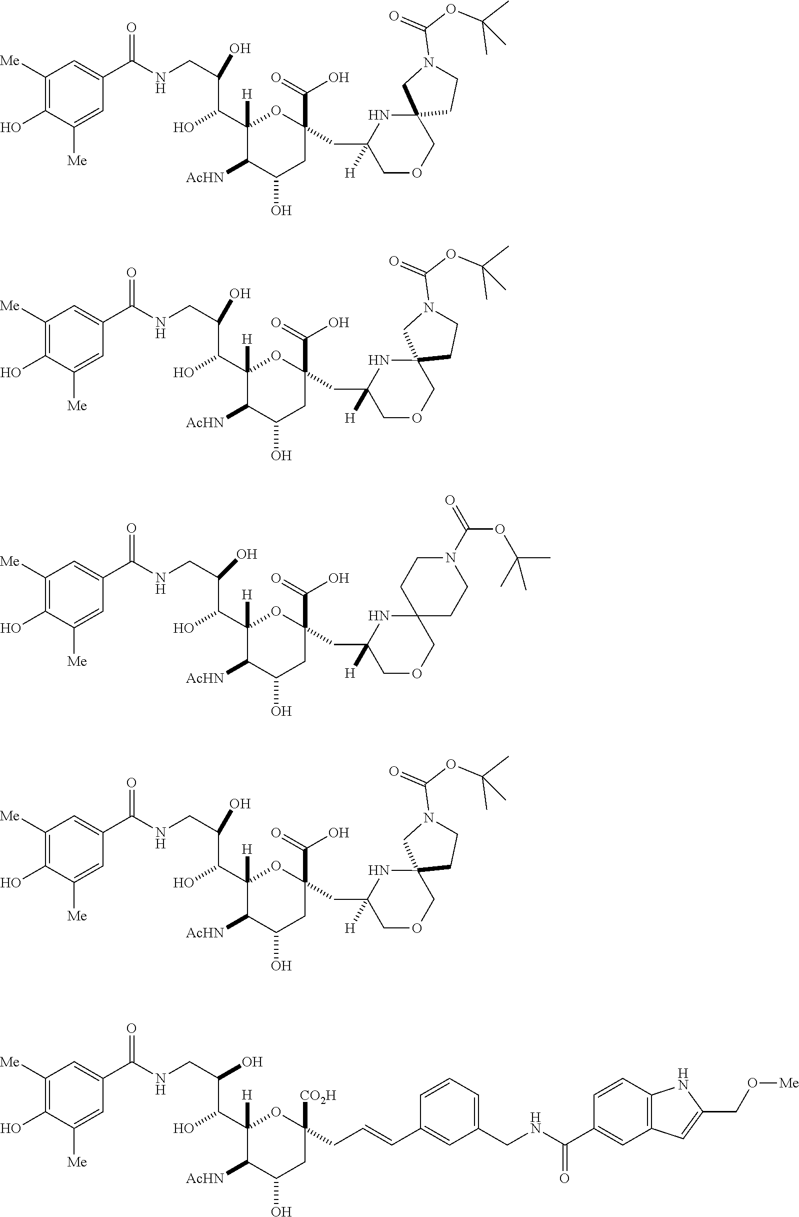 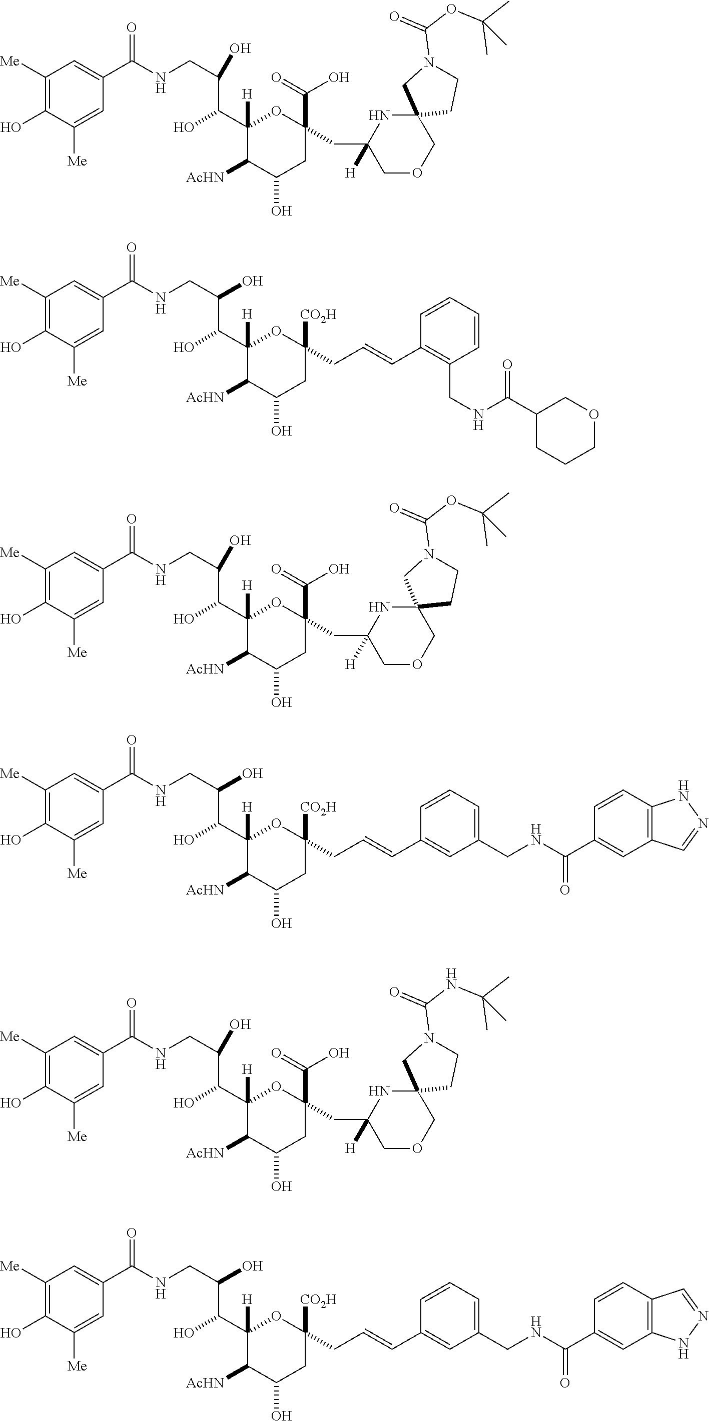 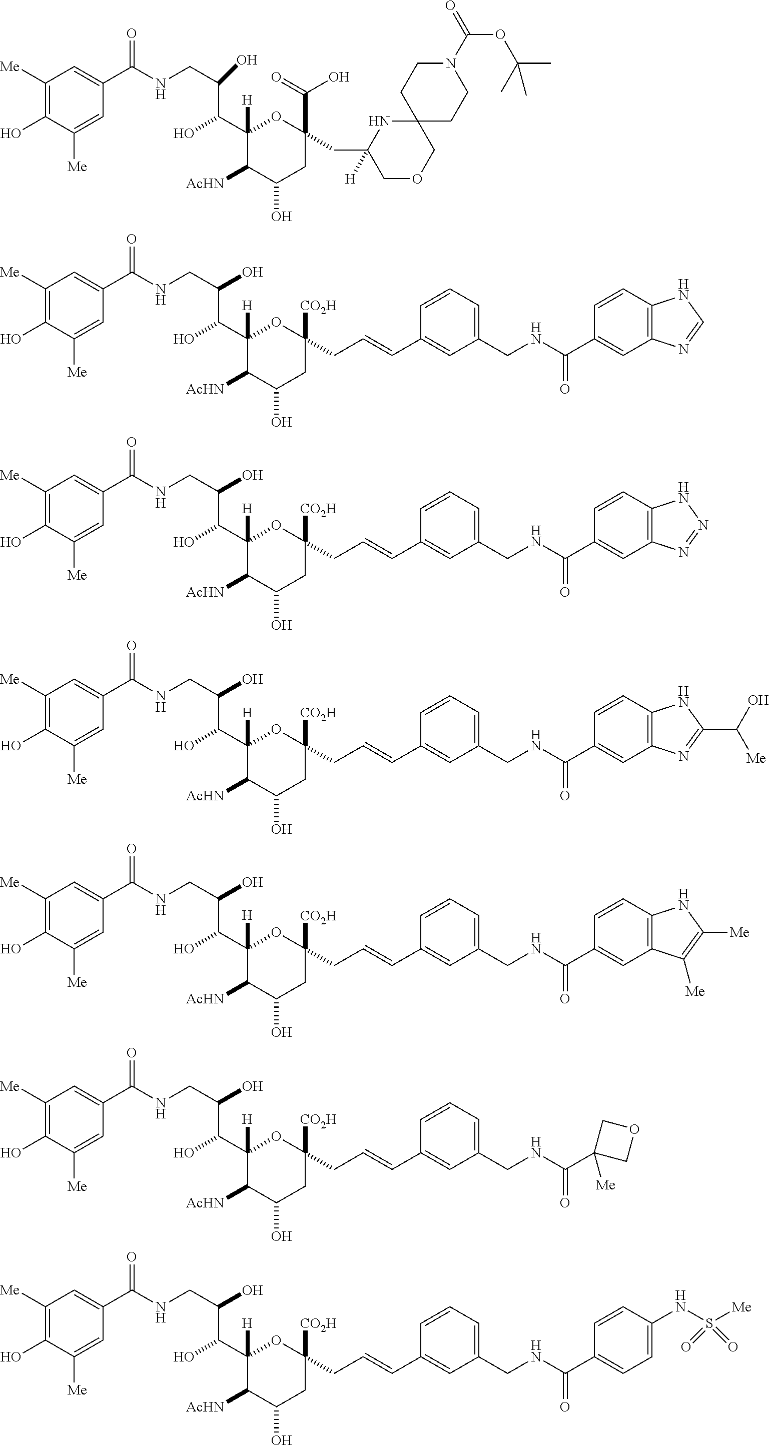 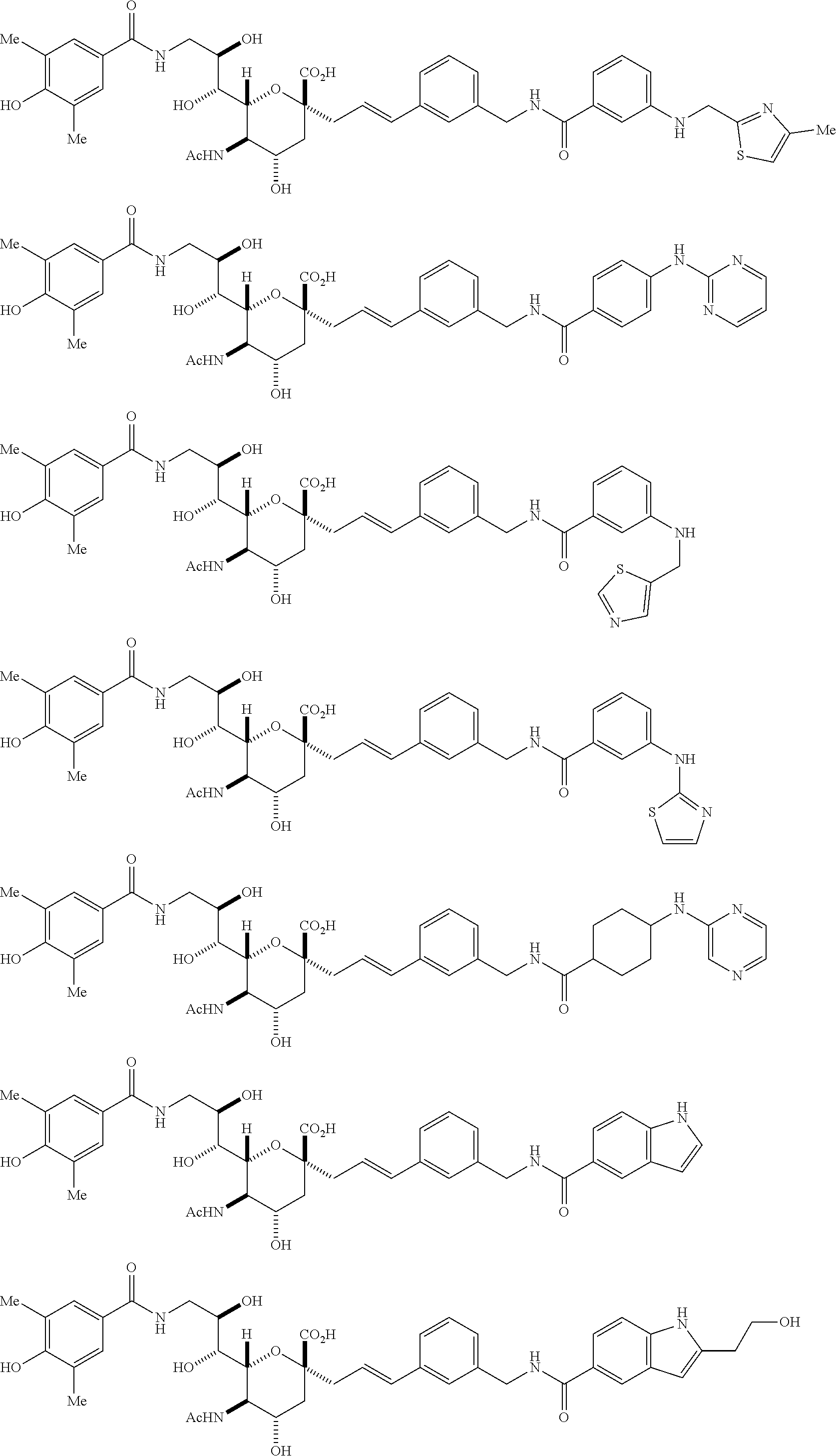 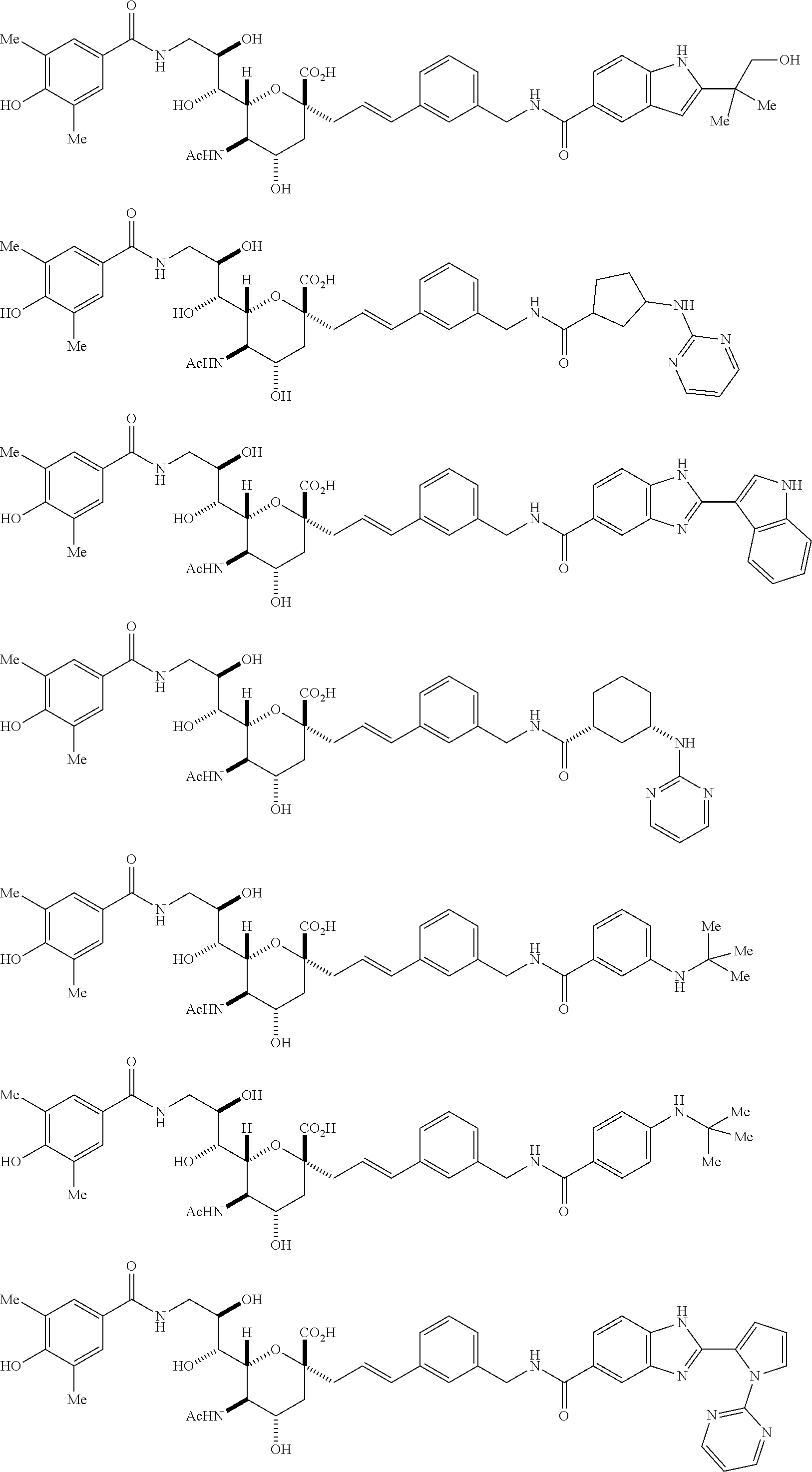 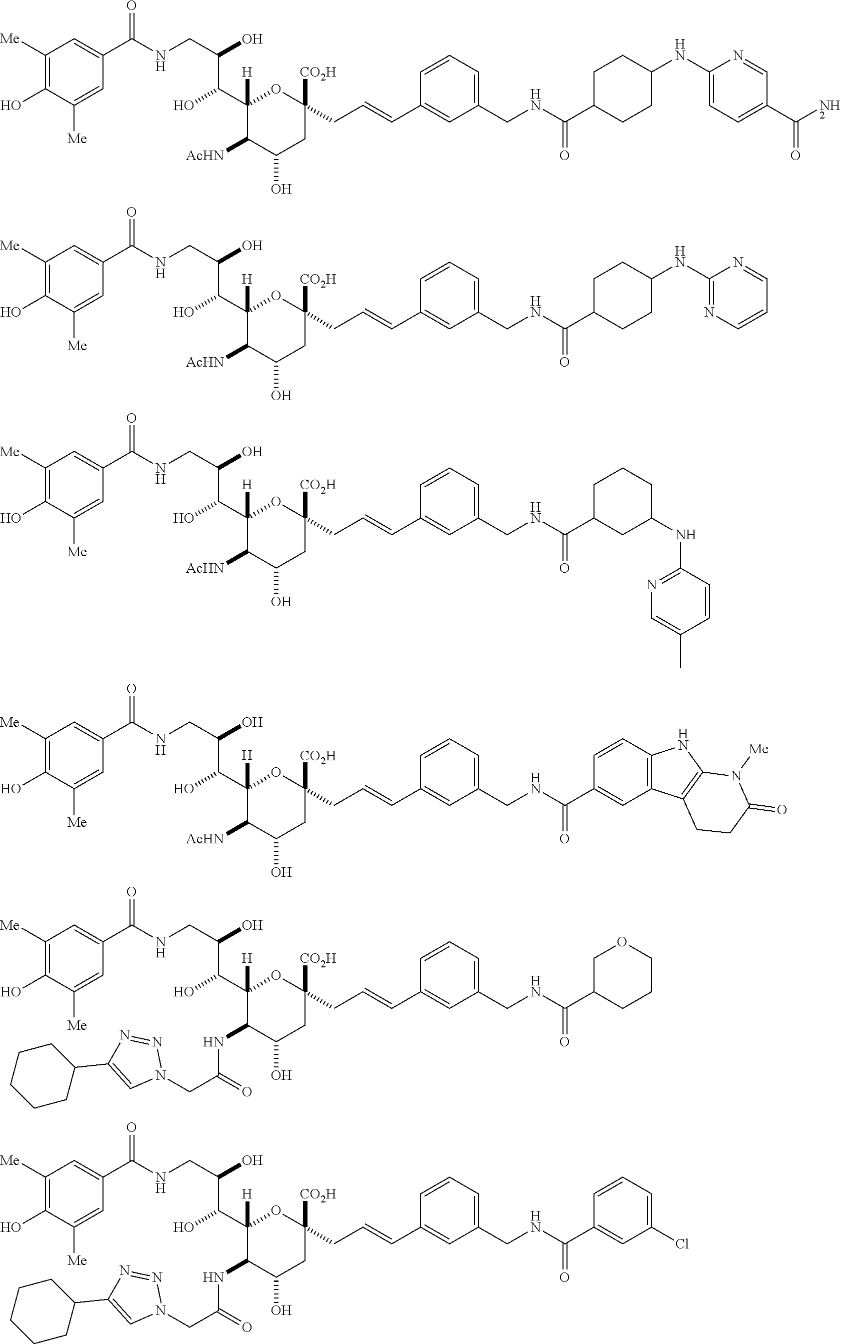 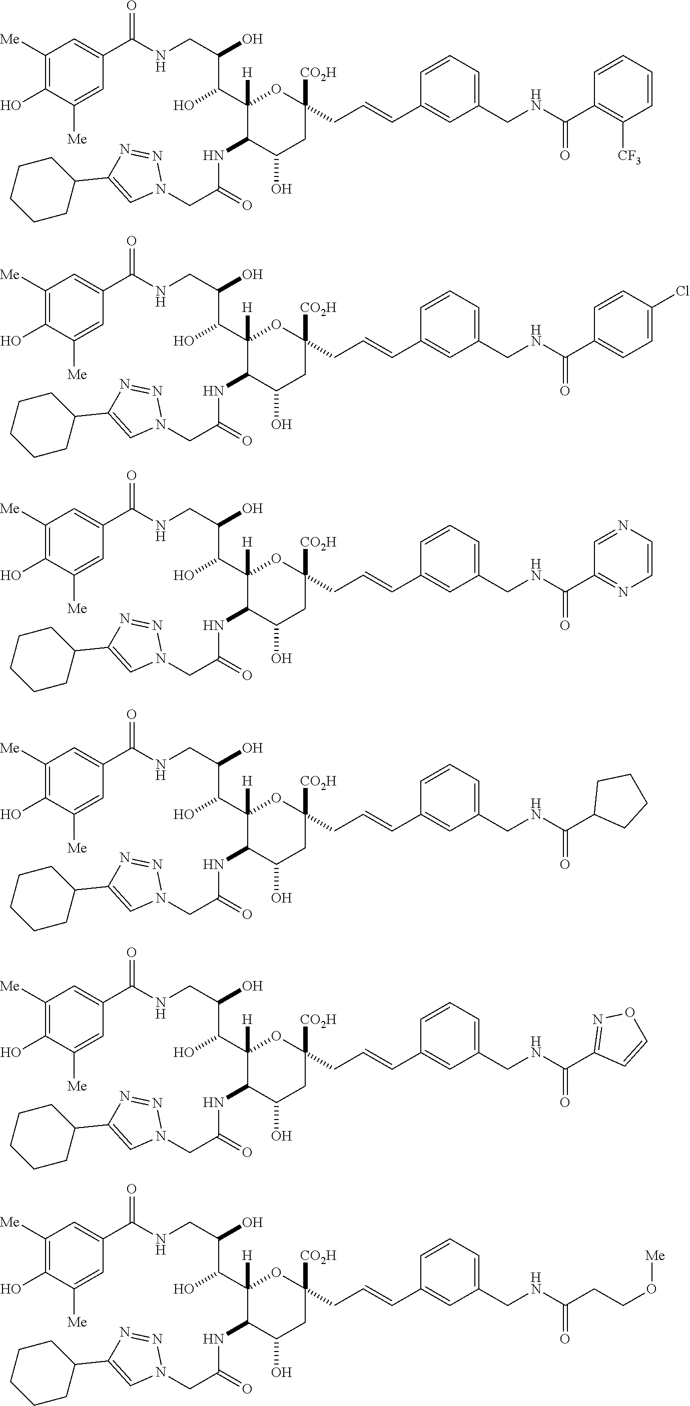 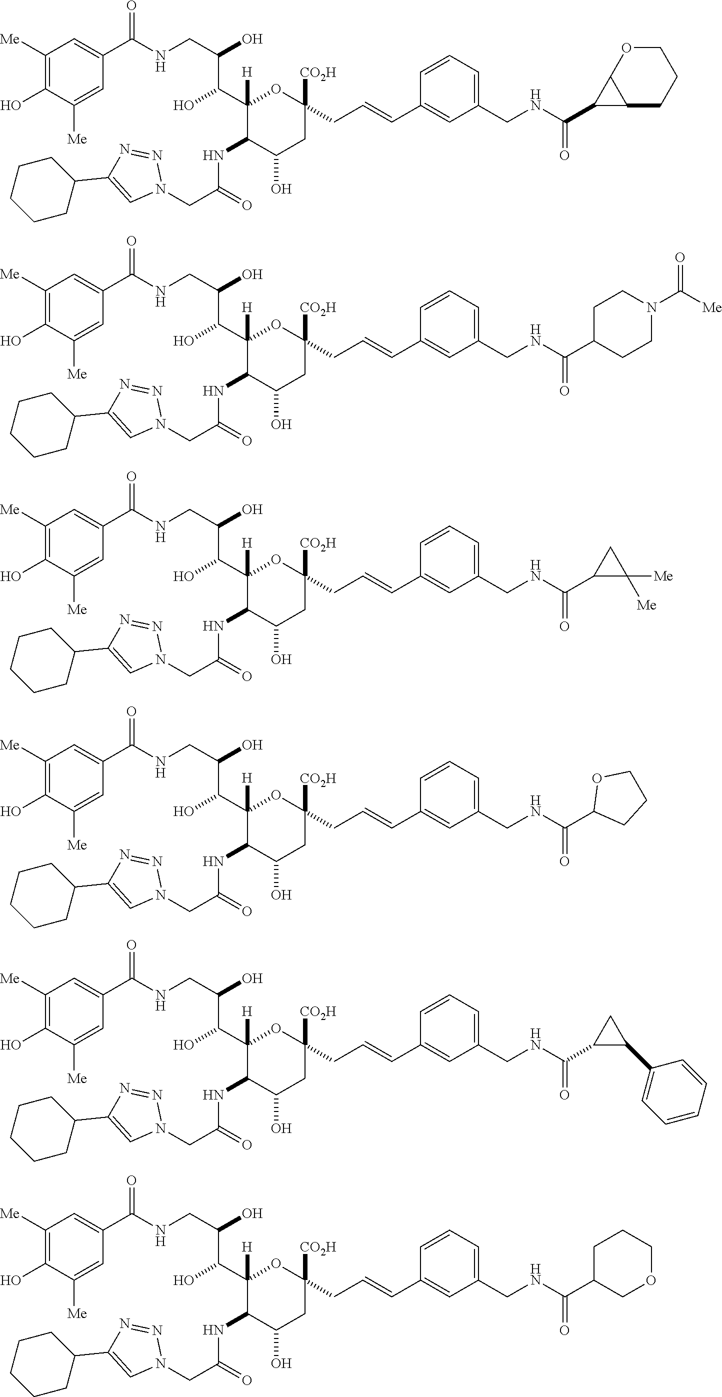 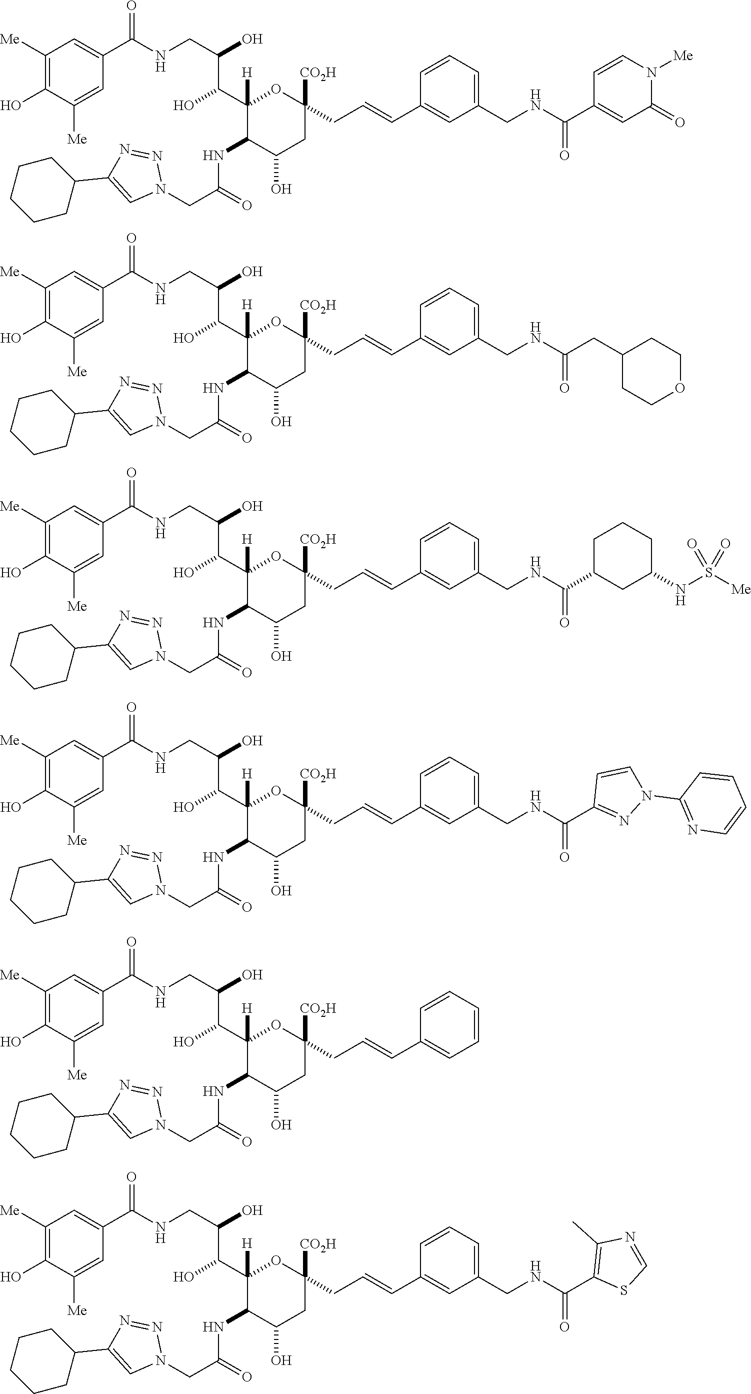 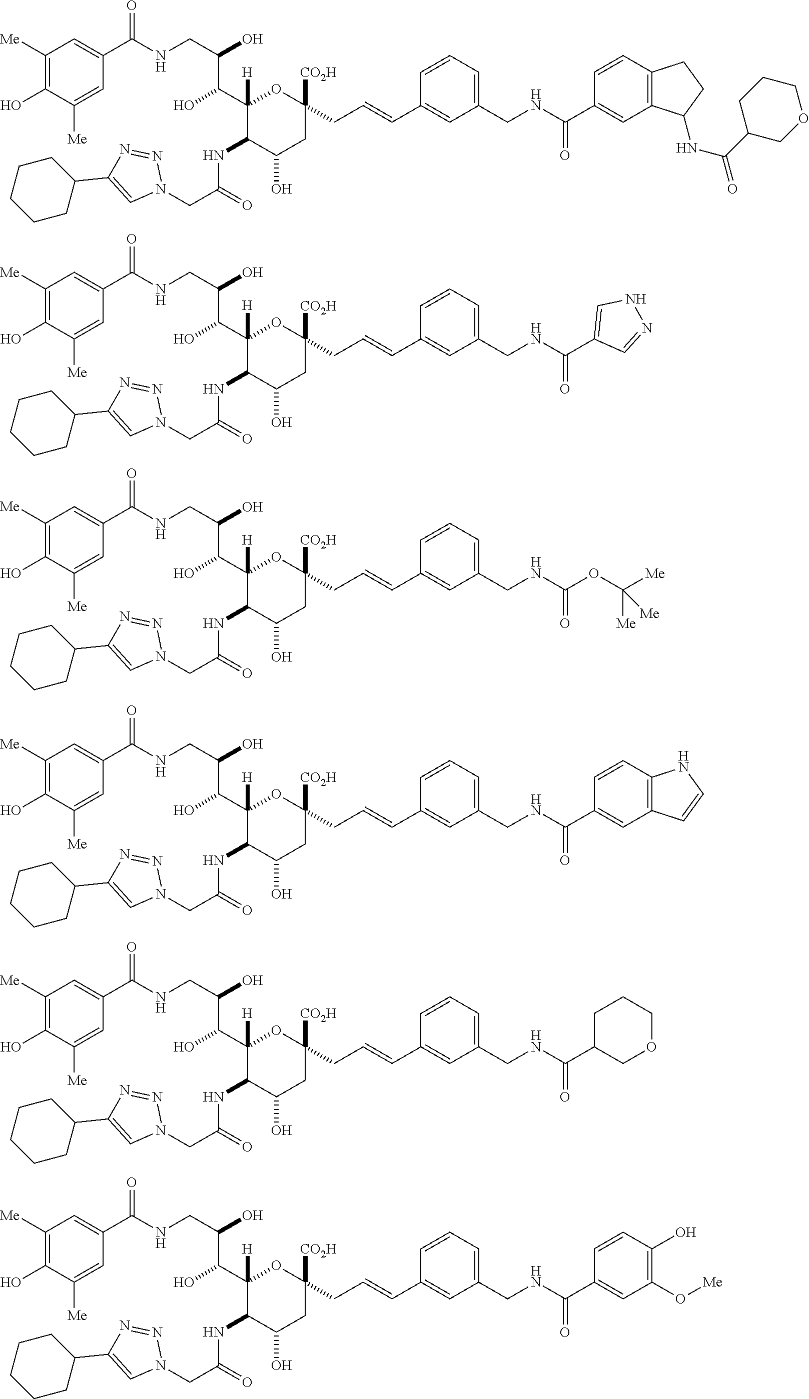 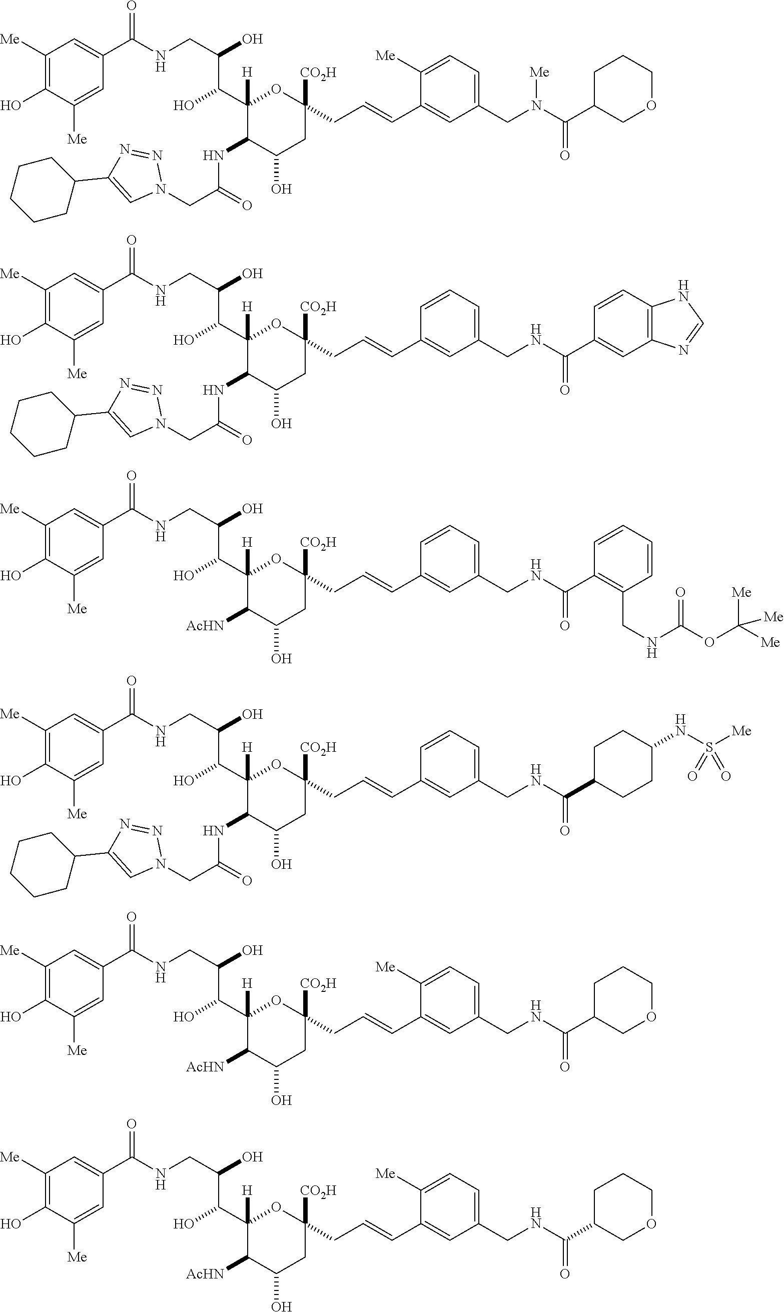 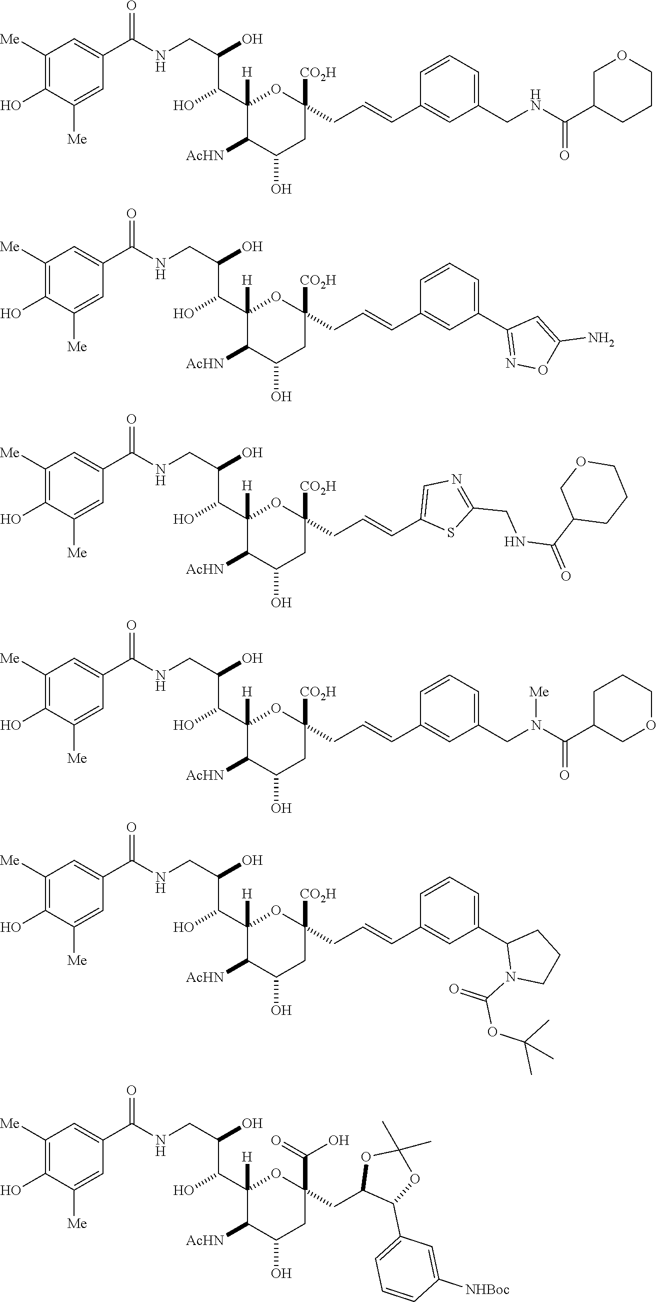 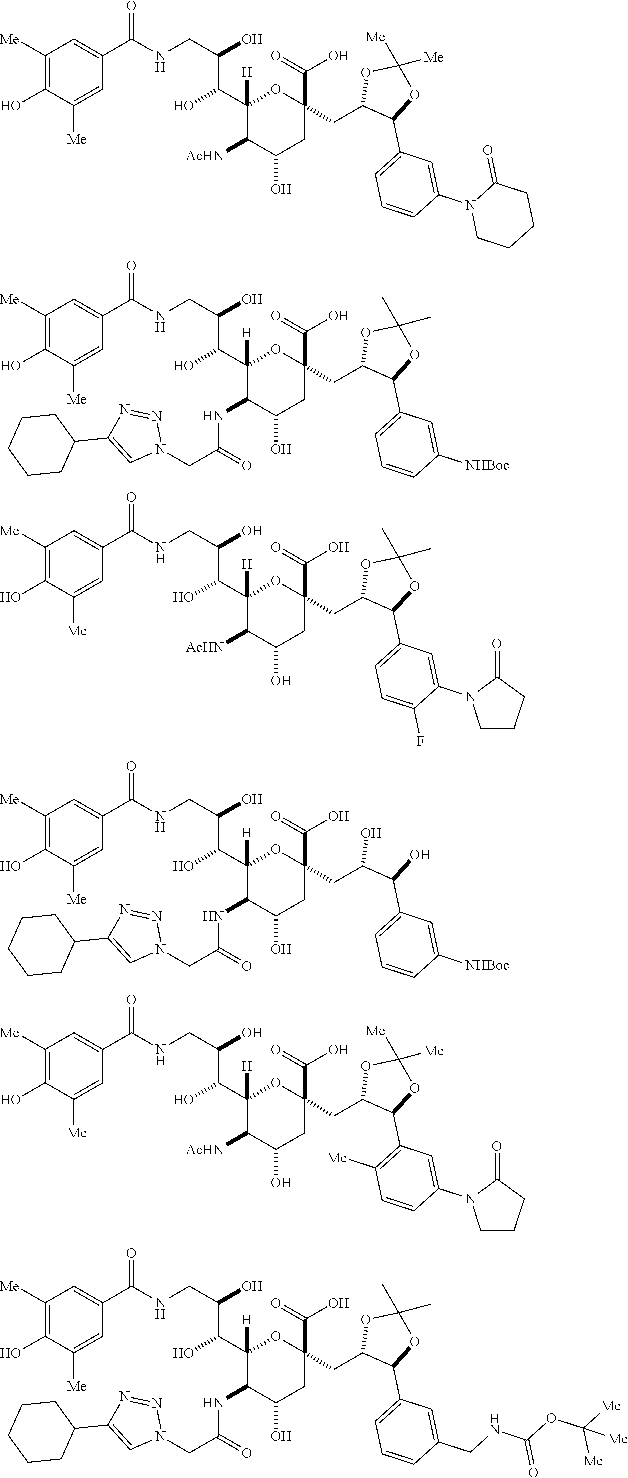 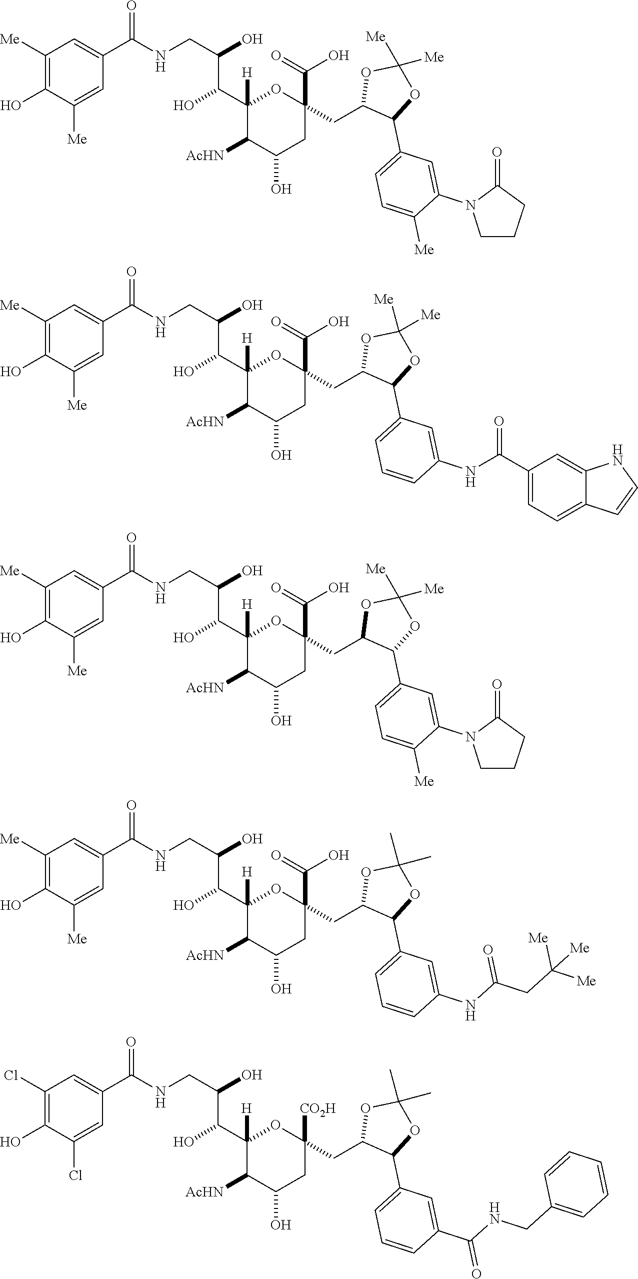 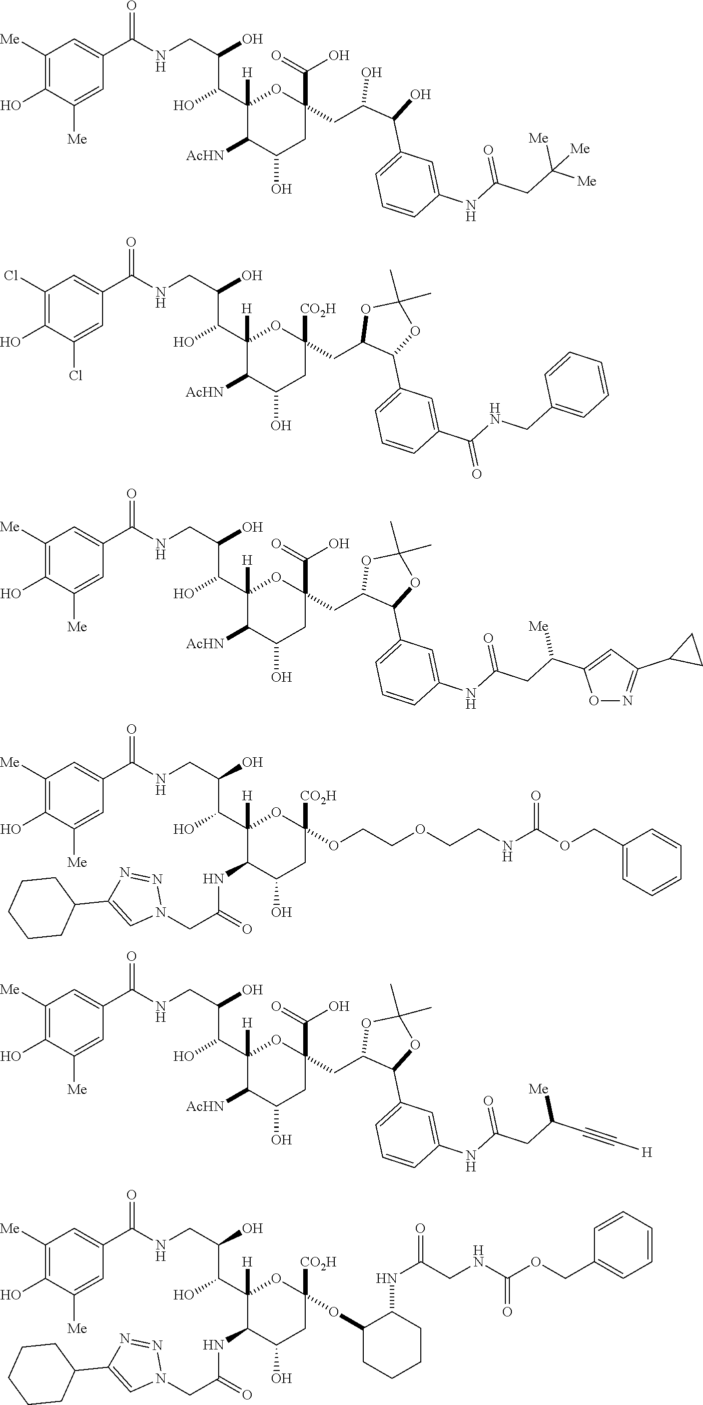 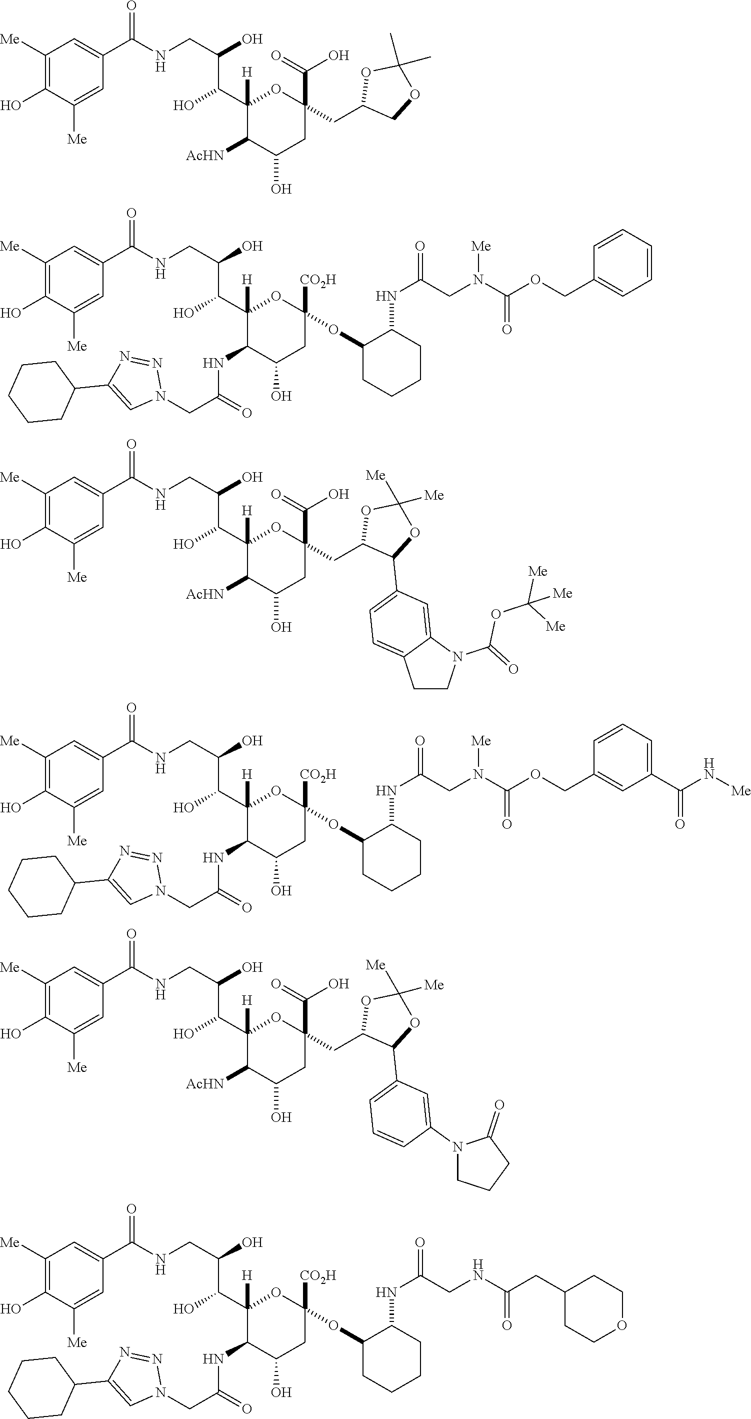 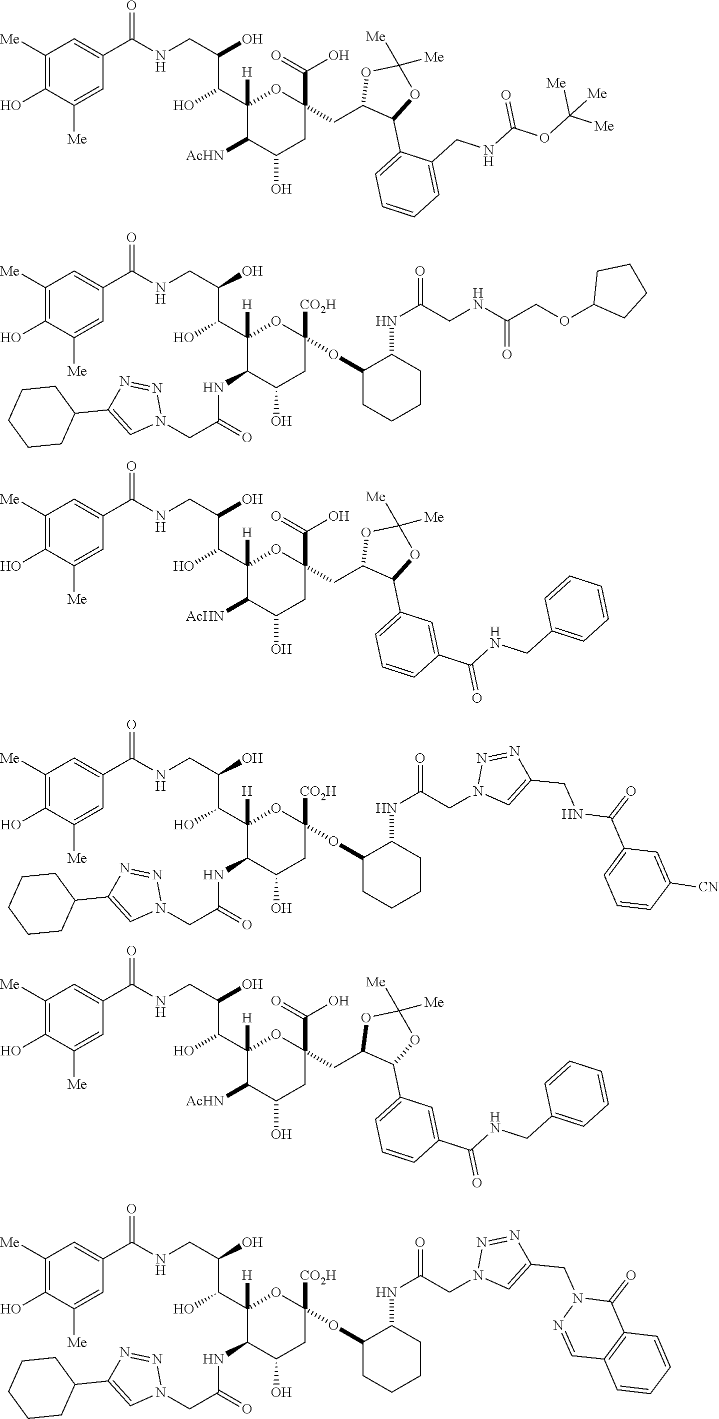 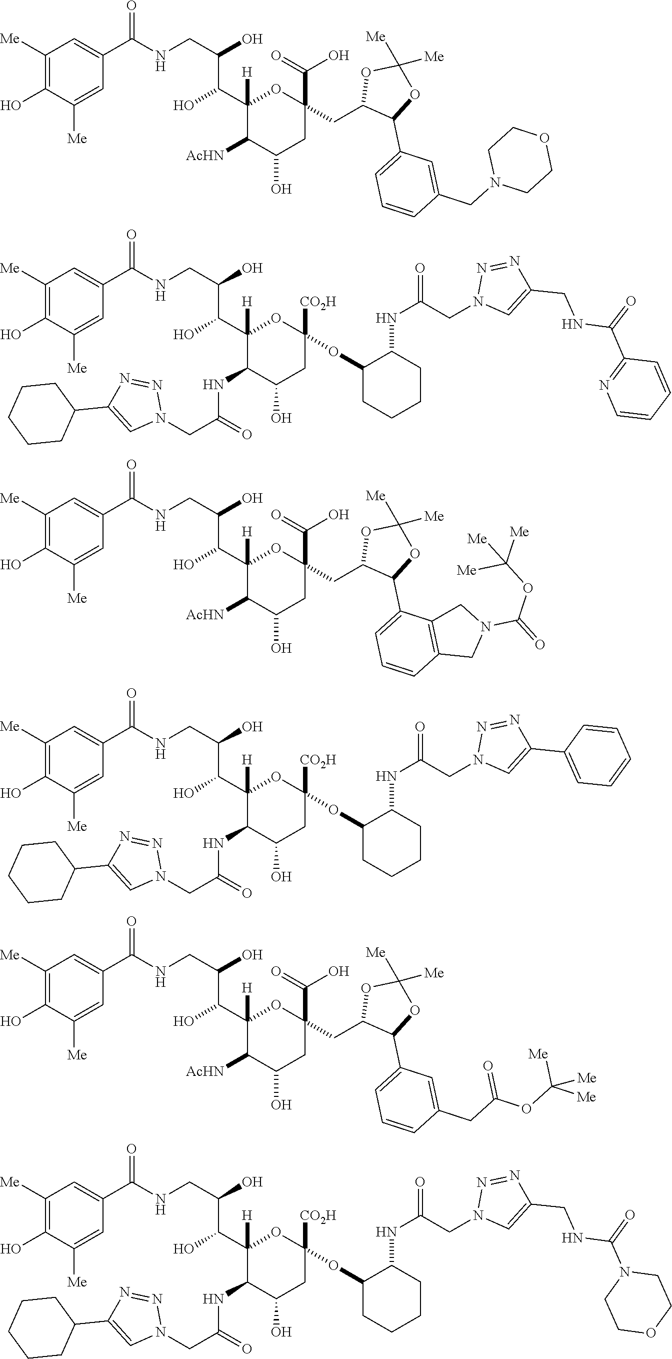 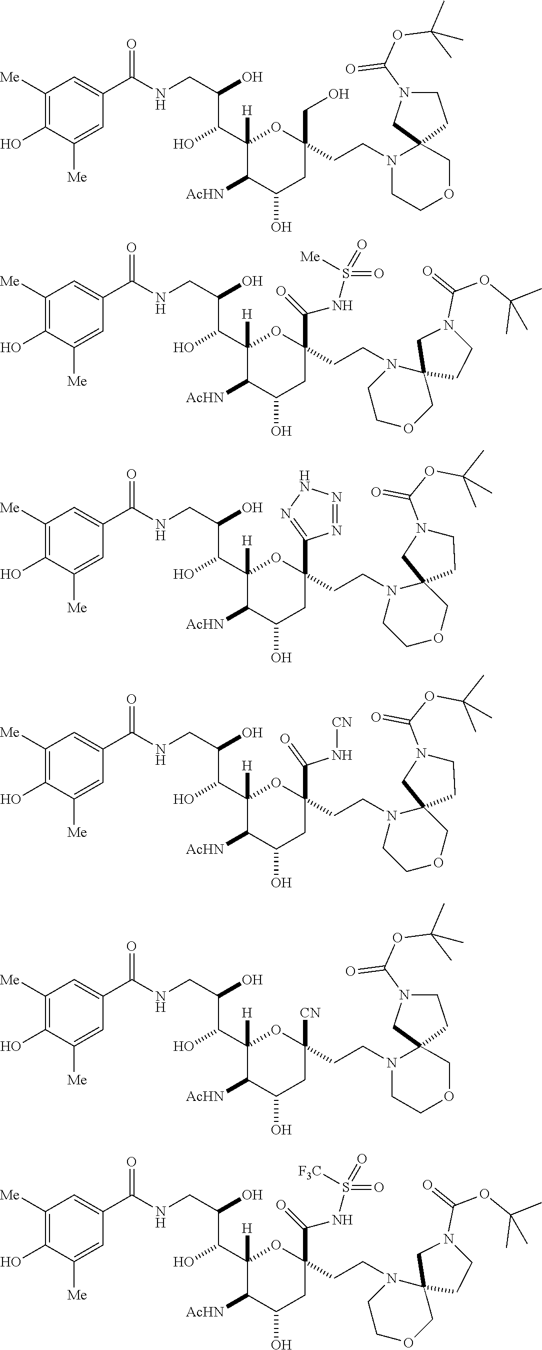 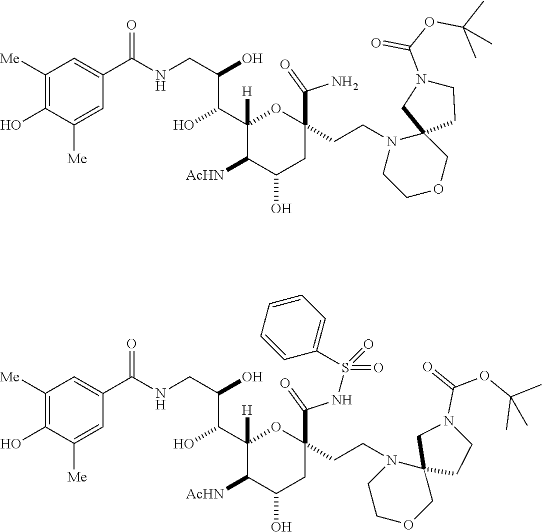
tautomers thereof, deuterated derivatives thereof, deuterated derivatives of tautomers thereof, or pharmaceutically acceptable salts of any of the foregoing.

45. A compound of Formula (II): 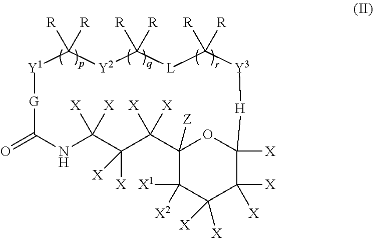
a tautomer thereof, a deuterated derivative of a compound of Formula (II), a deuterated derivative of a tautomer of a compound of Formula (II), or a pharmaceutically acceptable salt of any of the foregoing, wherein:
(i) G is chosen from cycloalkyl groups, heterocyclic groups, aryl groups, and heteroaryl groups;
(ii) Y¹ is absent or —O—;
(iii) Y² is absent or chosen from —O—, —NHC(O)—, and aryl groups;
(iv) Y³ is absent or chosen from —O—, and aryl groups;
(v) H is chosen from C₁₋₁₀ linear alkylene groups, C₃₋₁₀ branched alkylene groups, C₃₋₁₀cyclic alkylene groups, —C(O)—C₁₋₁₀ linear alkylene groups, —C(O)—C₃₋₁₀ branched alkylene groups, —C(O)—C₃₋₁₀ cyclic alkylene groups, C₁₋₁₀ linear alkylene-C(O)— groups, C₃₋₁₀ branched alkylene-C(O)— groups, C₃₋₁₀ cyclic alkylene-C(O)— groups, C₁₋₁₀ linear alkenylene groups, C₃₋₁₀ branched alkenylene groups, and C₃₋₁₀cyclic alkenylene groups;
(vi) p, q, and r are independently chosen from 0, 1, 2, 3, 4, 5, and 6;
(vii) each R is independently chosen from hydrogen, hydroxy groups, amino groups, linear alkyl groups, branched alkyl groups, cyclic alkyl groups, linear alkoxy groups, branched alkoxy groups, and cyclic alkoxy groups;
(viii) L absent or is chosen from: 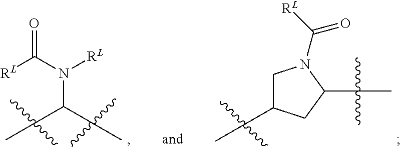
wherein R^{L} is chosen hydrogen, linear alkyl groups, branched alkyl groups, cyclic alkyl groups, heterocyclic groups, aryl groups, and heteroaryl groups;
(ix) each X is independently chosen from hydrogen, hydroxy groups, amino groups, linear alkyl groups, branched alkyl groups, and cyclic alkyl groups;
(x) X¹ and X² are each independently chosen from hydrogen, hydroxy groups, amino groups, linear alkyl groups, branched alkyl groups, cyclic alkyl groups, —NHC(O)alkylgroups, —NHC(O)arylalkylgroups, and —NHC(O)heteroarylalkylgroups;
(xi) Z is chosen from hydrogen, linear alkyl groups, branched alkyl groups, cyclic alkyl groups, heteroaryl groups, —CN, —CO₂H, —C(O)R^{z}, —C(O)NHCN, —CO₂R^{z}, —C(O)NHSO₂R^{z}; wherein R is chosen from hydrogen, linear alkyl groups, branched alkyl groups, cyclic alkyl groups, carbocyclic groups, amino groups, heterocyclic groups, aryl groups, and heteroaryl groups;
wherein the linear alkyl groups, branched alkyl groups, and cyclic alkyl groups, linear alkenyl groups, branched alkenyl groups, and cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C₃-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

46. A compound of Formula (IIa): 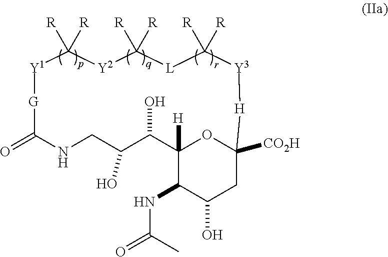
a tautomer thereof, a deuterated derivative of a compound of Formula (IIa), a deuterated derivative of a tautomer of a compound of Formula (IIa), or a pharmaceutically acceptable salt of any of the foregoing, wherein:
(i) G is chosen from cycloalkyl groups, heterocyclic groups, aryl groups, and heteroaryl groups;
(ii) Y¹ is absent or —O—;
(iii) Y² is absent or chosen from —O—, —NHC(O)—, and aryl groups;
(iv) Y³ is absent or chosen from —O—, and aryl groups;
(v) H is chosen from C₁₋₁₀ linear alkylene groups, C₃₋₁₀ branched alkylene groups, C₃₋₁₀ cyclic alkylene groups, —C(O)—C₁₋₁₀ linear alkylene groups, —C(O)—C₃₋₁₀ branched alkylene groups, —C(O)—C₃₋₁₀ cyclic alkylene groups, C₁₋₁₀ linear alkylene-C(O)— groups, C₃₋₁₀ branched alkylene-C(O)— groups, C₃₋₁₀ cyclic alkylene-C(O)— groups, C₁₋₁₀ linear alkenylene groups, C₃₋₁₀ branched alkenylene groups, and C₃₋₁₀cyclic alkenylene groups;
(vi) p, q, and r are independently chosen from 0, 1, 2, 3, 4, 5, and 6;
(vii) each R is independently chosen from hydrogen, hydroxy groups, amino groups, linear alkyl groups, branched alkyl groups, cyclic alkyl groups, linear alkoxy groups, branched alkoxy groups, and cyclic alkoxy groups;
(viii) L absent or is chosen from: 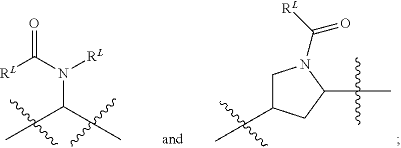
wherein R^{L} is chosen hydrogen, linear alkyl groups, branched alkyl groups, cyclic alkyl groups, heterocyclic groups, aryl groups, and heteroaryl groups;
wherein the linear alkyl groups, branched alkyl groups, cyclic alkyl groups, linear alkenyl groups, branched alkenyl groups, cyclic alkenyl groups, carbocyclic groups, linear heteroalkenyl groups, branched heteroalkenyl groups, heterocyclic groups, aryl groups, and heteroaryl groups are optionally substituted with at least one group chosen from halogen groups, hydroxy, thiol, amino, cyano, —C(O)OC₁-C₆ linear alkyl groups, —C(O)OC₃-C₆ branched alkyl groups, —C(O)OC₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —C(S)OC₁-C₆ linear alkyl groups, —C(S)OC₃-C₆ branched alkyl groups, —C(S)OC₃-C₆ cyclic alkyl groups, —C(S)NHC₁-C₆ linear alkyl groups, —C(S)NHC₃-C₆ branched alkyl groups, —C(S)NHC₃-C₆ cyclic alkyl groups, —C(O)O-arylalkyl groups, —C(O)O-heteroarylalkyl groups, —OC(O)C₁-C₆ linear alkyl groups, —OC(O)C₃-C₆ branched alkyl groups, —OC(O)C₃-C₆ cyclic alkyl groups, —NHC₁-C₆ linear alkyl groups, —NHC3-C₆ branched alkyl groups, —NHC₃-C₆ cyclic alkyl groups, —N(C₁-C₆ linear alkyl groups)₂, —N(C₃-C₆ branched alkyl groups)₂, —N(C₃-C₆ cyclic alkyl groups)₂, —NHC(O)C₁-C₆ linear alkyl groups, —NHC(O)C3-C₆ branched alkyl groups, —NHC(O)C₃-C₆ cyclic alkyl groups, —C(O)NHC₁-C₆ linear alkyl groups, —C(O)NHC₃-C₆ branched alkyl groups, —C(O)NHC₃-C₆ cyclic alkyl groups, —NHaryl groups, —N(aryl groups)₂, —NHC(O)aryl groups, —C(O)NHaryl groups, —NHheteroaryl groups, —N(heteroaryl groups)₂, —NHC(O)heteroaryl groups, —C(O)NHheteroaryl groups, C₁-C₆ linear alkyl groups, C₃-C₆ branched alkyl groups, C₃-C₆ cyclic alkyl groups, C₂-C₆ linear alkenyl groups, C₂-C₆ branched alkenyl groups, cyclic alkenyl groups, C₁-C₆ linear hydroxyalkyl groups, C₃-C₆ branched hydroxyalkyl groups, C₃-C₆ cyclic hydroxyalkyl groups, C₁-C₆ linear aminoalkyl groups, C₃-C₆ branched aminoalkyl groups, C₃-C₆ cyclic aminoalkyl groups, C₁-C₆ linear alkoxy groups, C₃-C₆ branched alkoxy groups, C₃-C₆ cyclic alkoxy groups, C₁-C₆ linear thioalkyl groups, C₃-C₆ branched thioalkyl groups, C₃-C₆ cyclic thioalkyl groups, C₁-C₆ linear haloalkyl groups, C₃-C₆ branched haloalkyl groups, C₃-C₆ cyclic haloalkyl groups, C₁-C₆ linear haloaminoalkyl groups, C₃-C₆ branched haloaminoalkyl groups, C₃-C₆ cyclic haloaminoalkyl groups, C₁-C₆ linear halothioalkyl groups, C₃-C₆ branched halothioalkyl groups, C₃-C₆ cyclic halothioalkyl groups, C₁-C₆ linear haloalkoxy groups, C₃-C₆ branched haloalkoxy groups, C₃-C₆ cyclic haloalkoxy groups, benzyloxy, benzylamino, and benzylthio groups, 3 to 6-membered heterocycloalkenyl groups, 3 to 6-membered heterocyclic groups, and 5 and 6-membered heteroaryl groups optionally substituted with 0, 1, or 2 C₁-C₆ linear alkyl groups, branched alkyl groups, and cyclic alkyl groups.

47. The compound of any of claims 45-46, wherein G is chosen from aryl groups.

48. The compound of claim 47, wherein G is

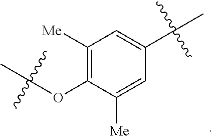 [SMILES: *Oc1c(C)cc(*)cc1C]

49. The compound of claim 47, wherein G is

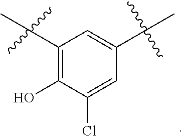 [SMILES: *c1cc(*)c(O)c(Cl)c1]

50. The compound of claim 45 or 46, chosen from: 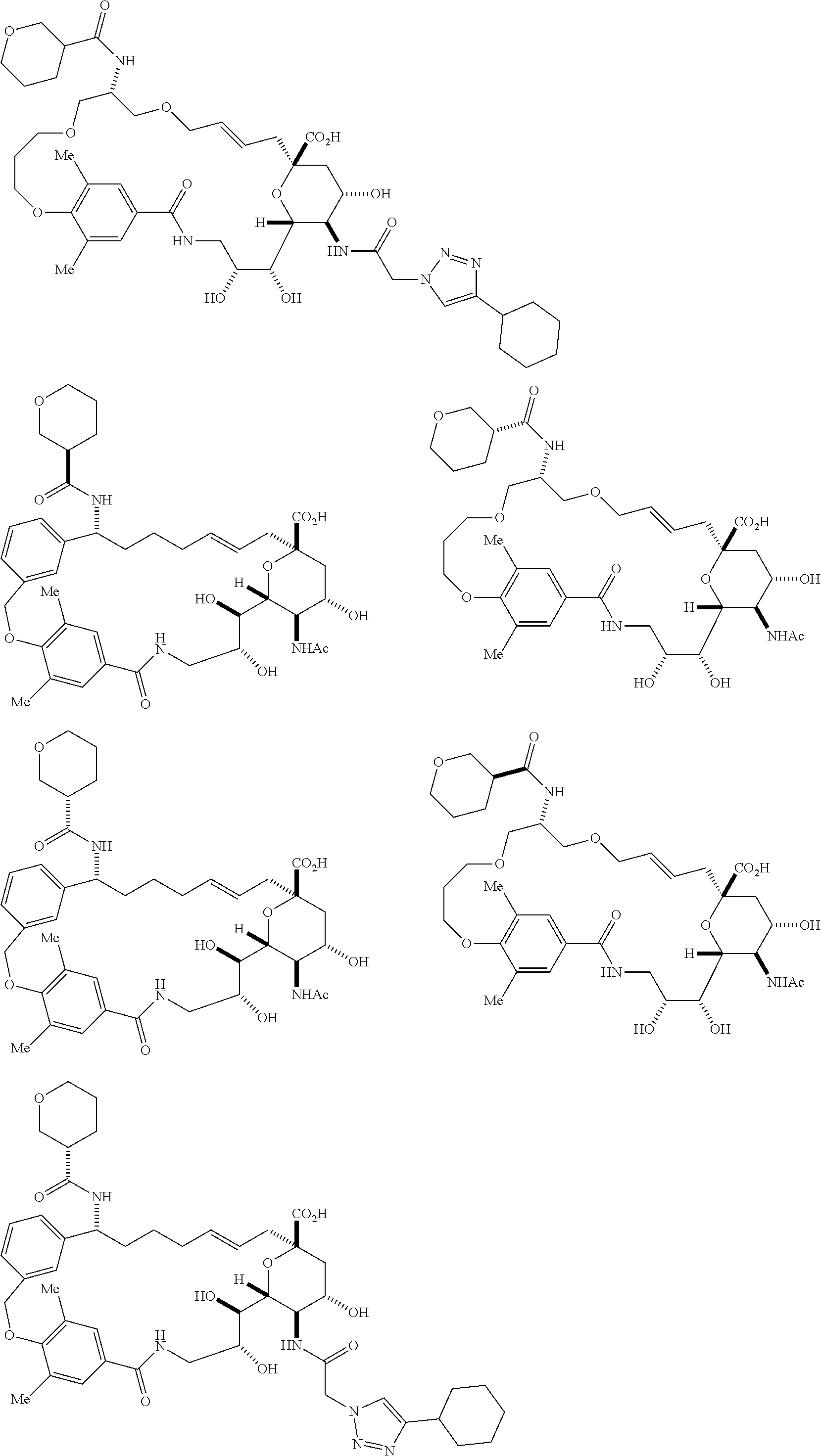 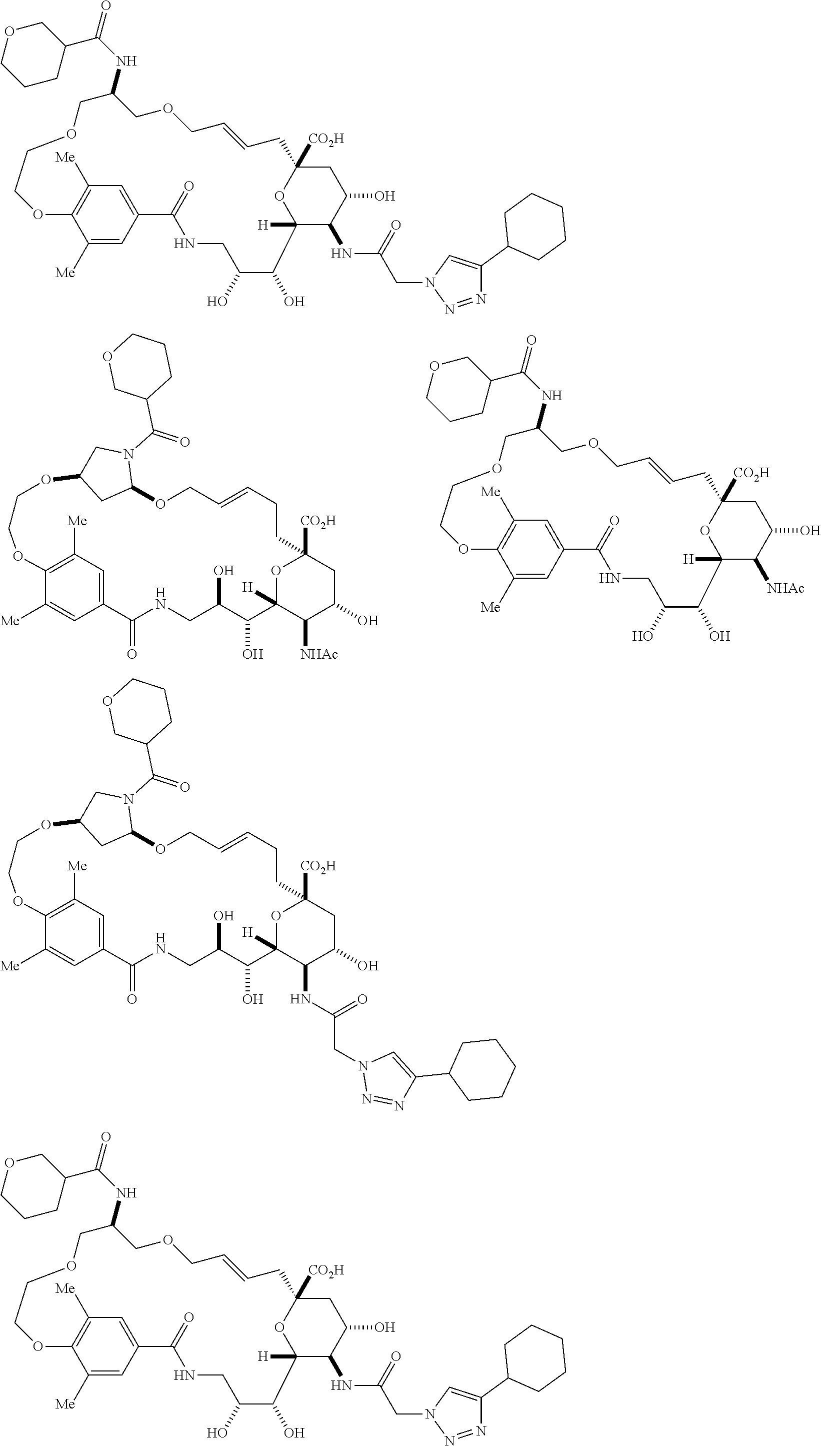 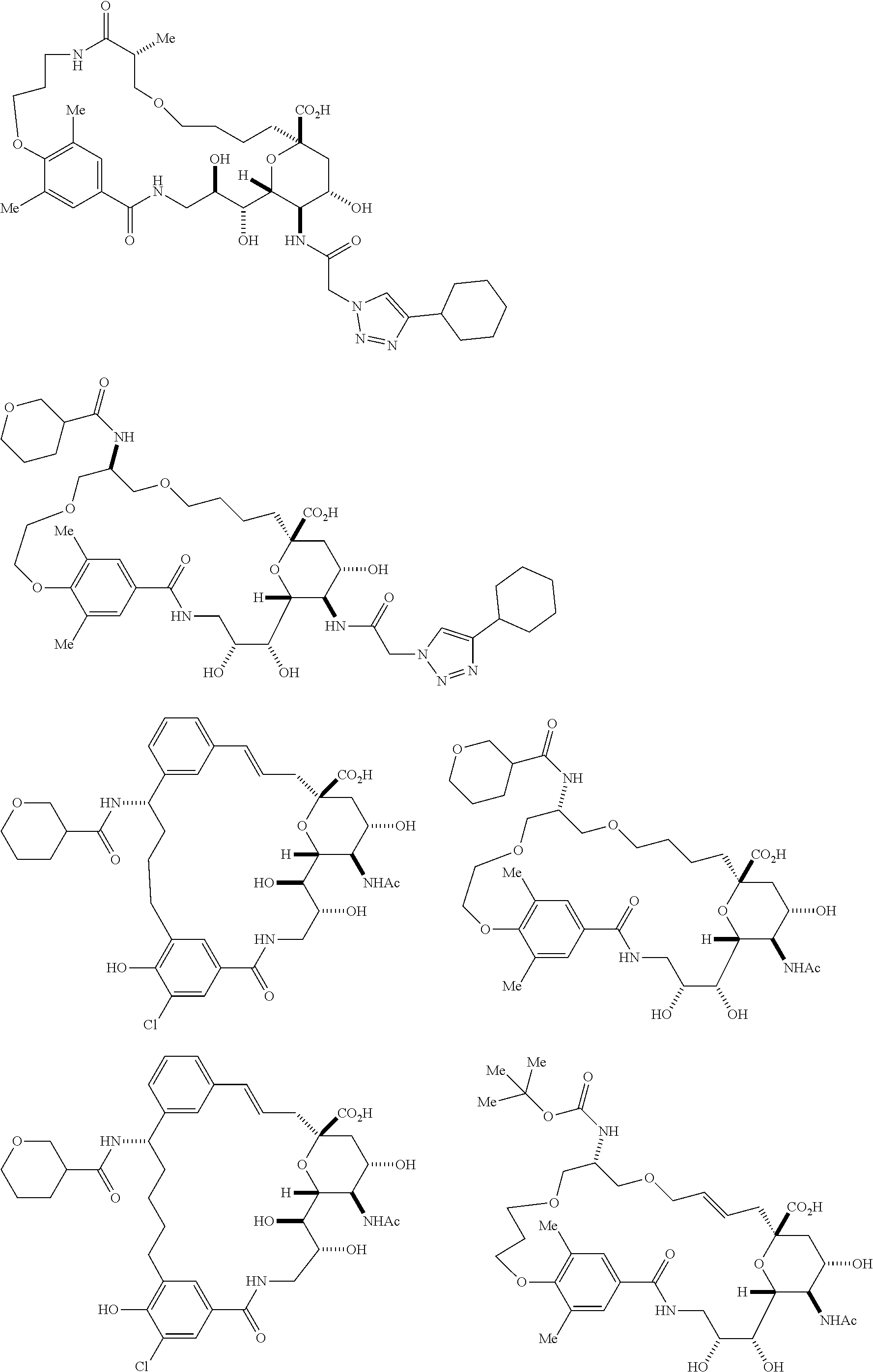 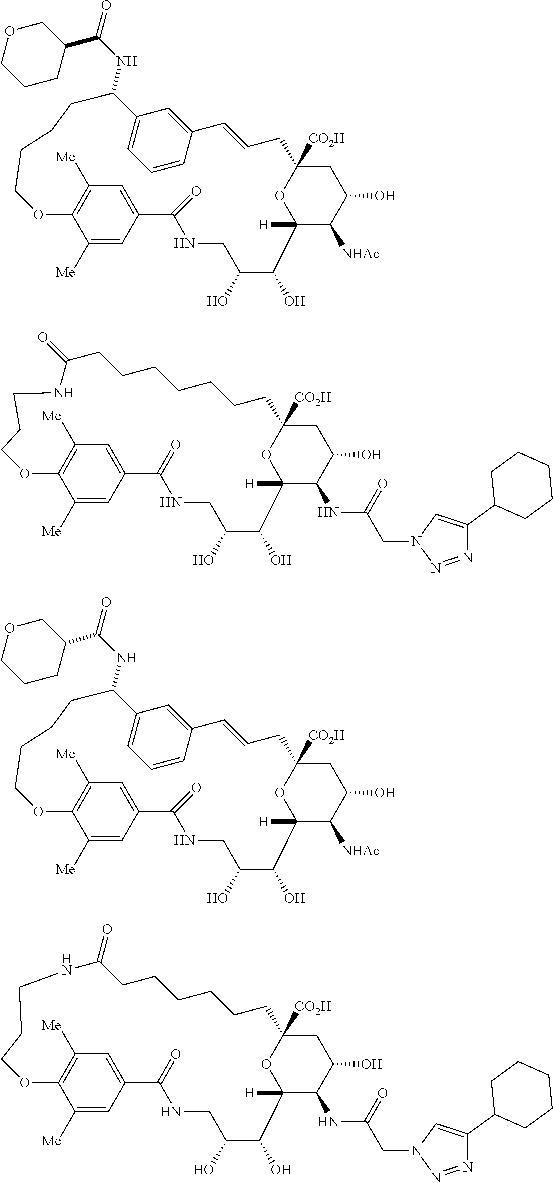 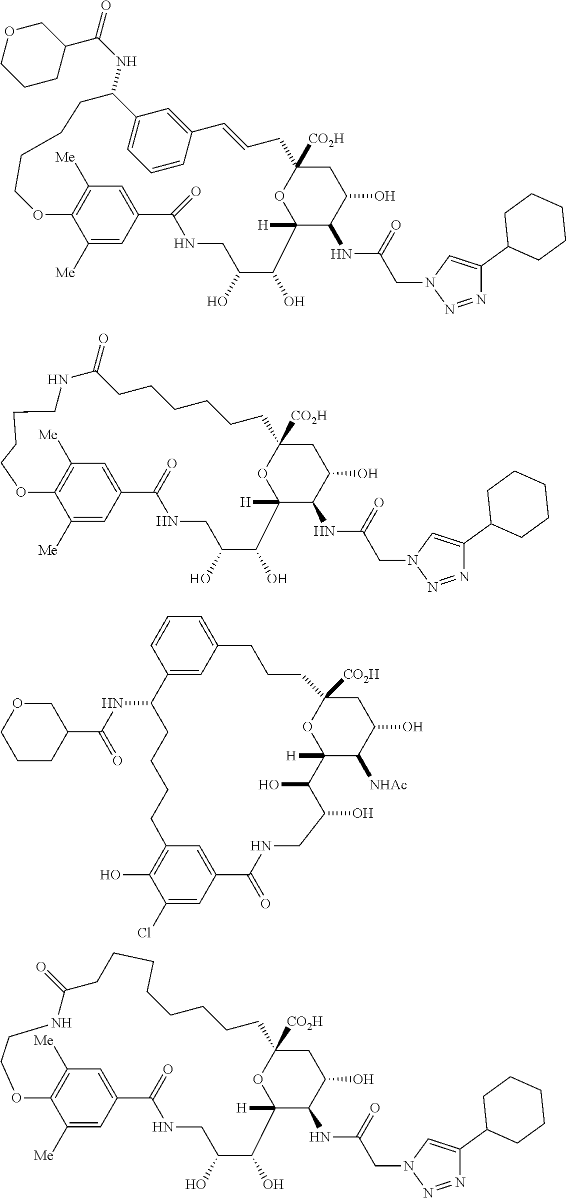
tautomers thereof, deuterated derivatives thereof, deuterated derivatives of tautomers thereof, and pharmaceutically acceptable salts of any of the foregoing.

51. A compound of Formula (III), (IV), or (V): 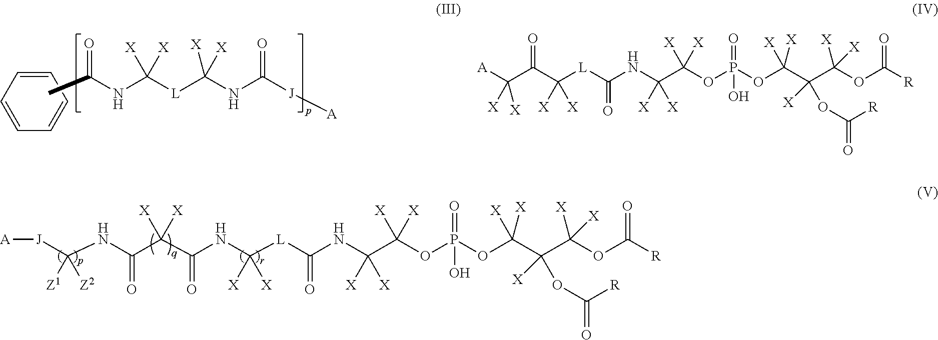
a tautomer thereof, a deuterated derivative of a compound of Formula (III), (IV), or (V), a deuterated derivative of a tautomer of a compound of Formula (III), (IV), or (V), or a pharmaceutically acceptable salt of any of the foregoing, wherein:
(i) A is a compound of any of claims 1-50;
(ii) J is chosen from cycloalkyl groups, heterocyclic groups, aryl groups, and heteroaryl groups;
(iii) Z¹, Z², and each X are each independently chosen from hydrogen, hydroxy groups, amino groups, linear alkyl groups, branched alkyl groups, and cyclic alkyl groups;
(iv) L is 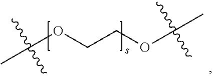
wherein s is 1-50.
(v) p, q, and r are independently chosen from 1, 2, 3, 4, 5, and 6.

52. The compound of Formula (III) of claim 51, wherein:
(i) A is a compound of any of claims 1-50;
(ii) J is chosen from cycloalkyl groups, heterocyclic groups, aryl groups, and heteroaryl groups;
(iii) each X is independently chosen from hydrogen, hydroxy groups, amino groups, linear alkyl groups, branched alkyl groups, and cyclic alkyl groups;
(iv) L is 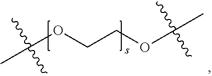
wherein s is 1-10; and
(v) p is chosen from 1, 2, and 3.

53. The compound of Formula (IV), wherein:
(i) A is a compound of any of claims 1-50;
(ii) J is chosen from cycloalkyl groups, heterocyclic groups, aryl groups, and heteroaryl groups;
(iii) each X is independently chosen from hydrogen, hydroxy groups, amino groups, linear alkyl groups, branched alkyl groups, and cyclic alkyl groups;
(iv) L is 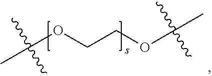
wherein s is 10-50; and
(v) p, q, and r are independently chosen from 1, 2, 3, 4, 5, and 6.

54. The compound of Formula (V), wherein:
(i) A is a compound of any of claims 1-50;
(ii) J is chosen from cycloalkyl groups, heterocyclic groups, aryl groups, and heteroaryl groups;
(iii) Z¹, Z², and each X are independently chosen from hydrogen, hydroxy groups, amino groups, linear alkyl groups, branched alkyl groups, and cyclic alkyl groups;
(iv) L is 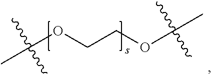
wherein s is 10-50; and
(v) p, q, and r are independently chosen from 1, 2, 3, 4, 5, and 6.

55. A compound of any one of claims 51-54, wherein J is absent or a cyclohexyl group.

56. The compound of Formula (III), wherein the compound is: 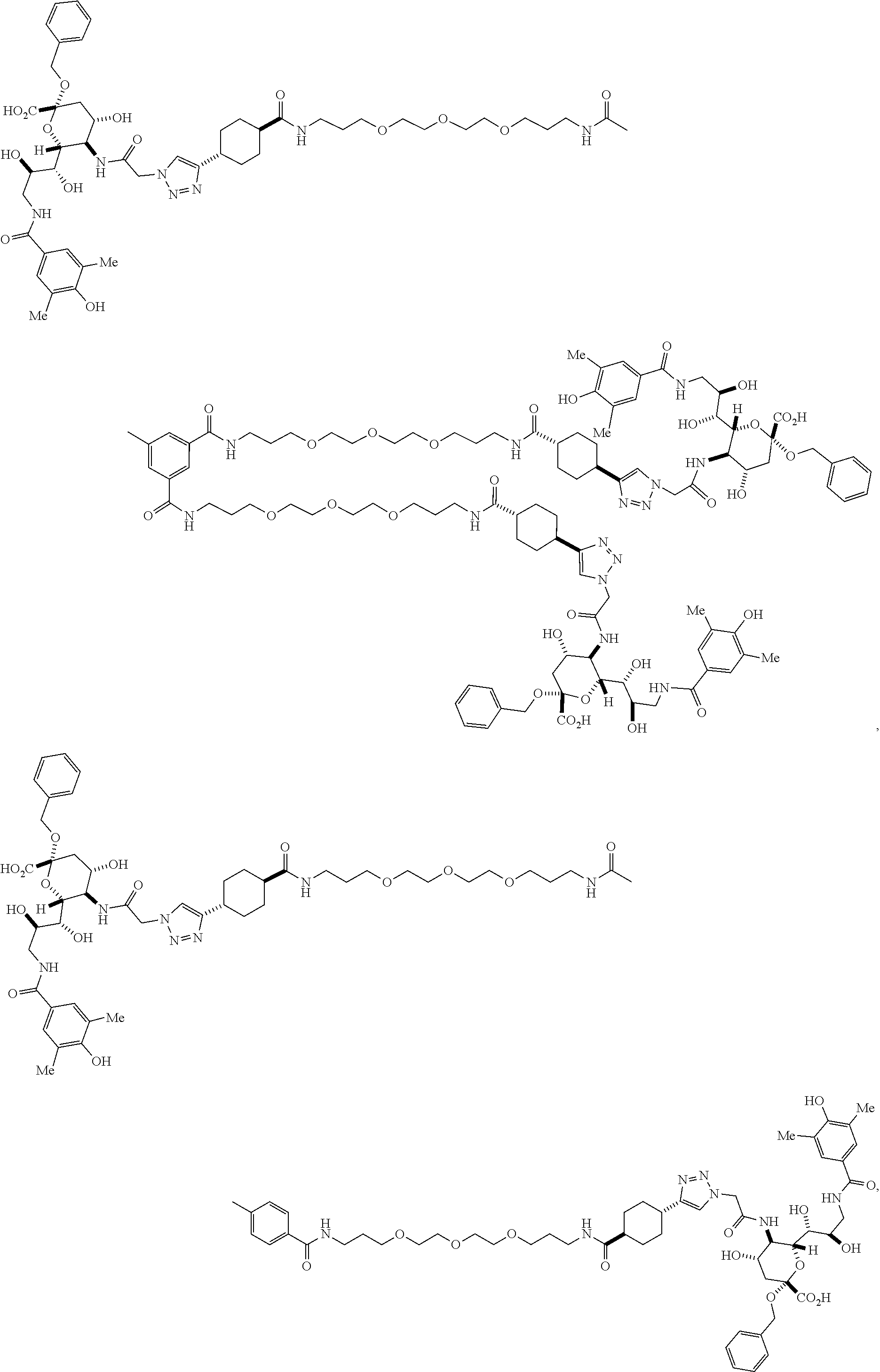
a tautomer thereof, a deuterated derivative thereof, a deuterated derivative of a tautomer thereof, or a pharmaceutically acceptable salt of any of the foregoing.

57. A compound of Formula (IV), wherein the compound is: 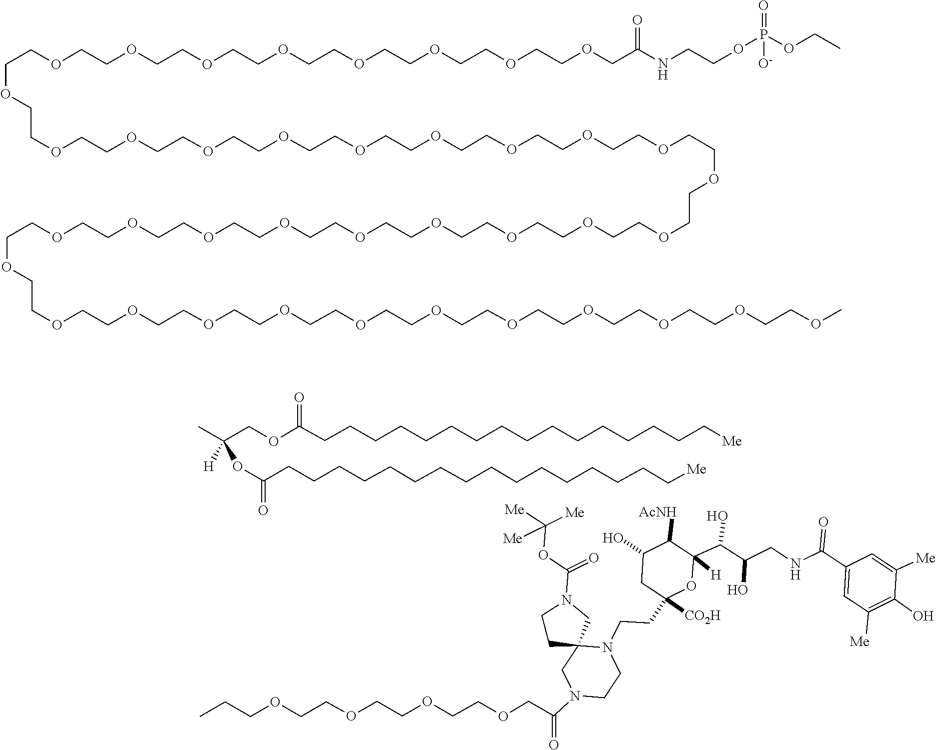
a tautomer thereof, a deuterated derivative thereof, a deuterated derivative of a tautomer thereof, or a pharmaceutically acceptable salt of any of the foregoing.

58. A compound of Formula (V), wherein the compound is: 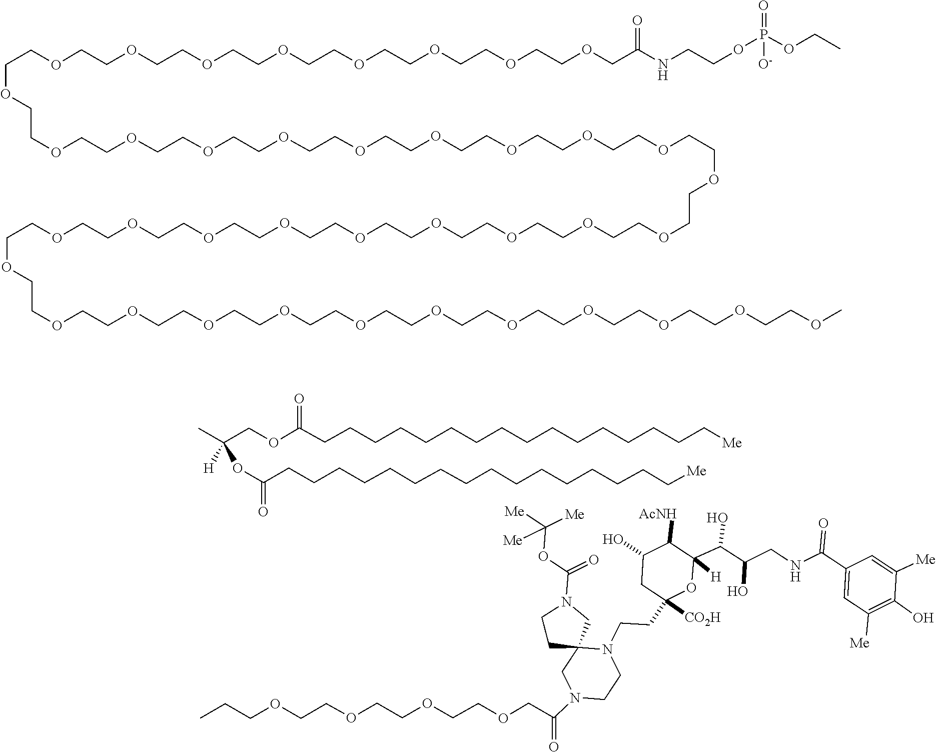
a tautomer thereof, a deuterated derivative thereof, a deuterated derivative of a tautomer thereof, or a pharmaceutically acceptable salt of any of the foregoing.
